SEQUENCE LISTING (35 sequences)

SEQ ID NO: 1 - protein, 7 aa, synthetic construct
TSFLIHW

SEQ ID NO: 2 - protein, 16 aa, synthetic construct
FIIPGNGGTKYNQKFQ

SEQ ID NO: 3 - protein, 11 aa, synthetic construct
YDGSFEGYFDV

SEQ ID NO: 4 - protein, 15 aa, synthetic construct
SSQSLVHSDGNTYLH

SEQ ID NO: 5 - protein, 7 aa, synthetic construct
KVSNRDS

SEQ ID NO: 6 - protein, 9 aa, synthetic construct
SQSTHWPWT

SEQ ID NO: 7 - protein, 54 aa, synthetic construct
MLQMAGQCSQNEYFDSLLHACIPCQLRCSSNTPPLTCQRYCNASVTNSVKGTNA

SEQ ID NO: 8 - protein, 450 aa, synthetic construct
QVQLVQSGAEVVKPGASVKMSCKASGYTFTSFLIHWVKQAPGQGLEWIGFIIPGNGGTKYNQKFQGKATLTSDTSSSTAYMELSSLRSEDSAVYYCARYDGSFEGYFDVWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DVVMTQSPLSLPVSLRQPASISCRSSQSLVHSDGNTYLHWYLQKPGQSPRLLIYKVSNRDSGVPDRFSGSGSGTDFTLKISRVEAEDLGVYFCSQSTHWPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 121 aa, synthetic construct
EVQLQQSGPELVKPGASVKMSCKASGYTFTSFLIHWVKQKPGQGLEWIGFIIPYNDGTKYNEKFKGKATLTSDKSSSTAYMELSSLTSEDSAVYYCARYDGSFEGYFDVWGAGTTLTVSSA

SEQ ID NO: 11 - protein, 112 aa, synthetic construct
DVLMTQTPLSLPVSLRDQASISCRSSQSLVHSDGNTYLHWYLQKPGQSPKLLIYKVSNRFSGVPDRFSGSGSGTDFTLKISRVEAEDLGVYFCSQSTHVPWTFGGGTKLEIK

SEQ ID NO: 12 - protein, 329 aa, synthetic construct
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 450 aa, synthetic construct
EVQLQQSGPELVKPGASVKMSCKASGYTFTSFLIHWVKQKPGQGLEWIGFIIPYNDGTKYNEKFKGKATLTSDKSSSTAYMELSSLTSEDSAVYYCARYDGSFEGYFDVWGAGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 218 aa, synthetic construct
DVLMTQTPLSLPVSLRDQASISCRSSQSLVHSDGNTYLHWYLQKPGQSPKLLIYKVSNRFSGVPDRFSGSGSGTDFTLKISRVEAEDLGVYFCSQSTHVPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 121 aa, synthetic construct
QVQLVQSGAEVVKPGASVKMSCKASGYTFTSFLIHWVKQAPGQGLEWIGFIIPGNGGTKYNQKFQGKATLTSDTSSSTAYMELSSLRSEDSAVYYCARYDGSFEGYFDVWGQGTTLTVSSA

SEQ ID NO: 17 - protein, 113 aa, synthetic construct
DVVMTQSPLSLPVSLRQPASISCRSSQSLVHSDGNTYLHWYLQKPGQSPRLLIYKVSNRDSGVPDRFSGSGSGTDFTLKISRVEAEDLGVYFCSQSTHWPWTFGQGTKLEIKR

SEQ ID NO: 18 - DNA, 1410 bp, synthetic construct
  source 1..1410: mol_type = other DNA
atggagttcggcctgtcctgggtgttcctggtggccctgtttaggggagtgcagtgccaggtgcagctggtgcagagcggagctgaggtggtgaagccaggagctagcgtgaagatgtcttgtaaggcctccggctacaccttcacatcttttctgatccactgggtgaagcaggctccaggacagggactggagtggatcggcttcatcatccctggcaacggcggcaccaagtacaatcagaagtttcagggcaaggccaccctgacatccgacacctccagctctacagcttatatggagctgtccagcctgaggtctgaggattccgccgtgtactattgcgctcggtacgacggcagcttcgagggctattttgacgtgtggggccagggcaccacactgacagtgtctagcgcttctaccaagggaccatccgtgttcccactggccccctccagcaagtccaccagcggaggaacagccgctctgggatgcctggtgaaggactacttcccagagcccgtgacagtgagctggaactctggcgctctgacctccggcgtgcacacatttccagctgtgctgcagtcttccggcctgtacagcctgagctctgtggtgaccgtgccctccagctctctgggcacccagacatatatctgcaacgtgaatcacaagccatccaatacaaaggtggacaagaaggtggagcccaagagctgtgataagacccatacatgccccccttgtcctgccccagagctgctgggaggaccatccgtgttcctgtttccacccaagcctaaggacaccctgatgatctctaggacccccgaggtgacatgcgtggtggtggacgtgtcccacgaggaccccgaggtgaagtttaactggtacgtggatggcgtggaggtgcataatgctaagaccaagcctagggaggagcagtacaacagcacctatcgggtggtgtctgtgctgacagtgctgcaccaggattggctgaacggcaaggagtataagtgcaaggtgagcaataaggccctgcccgctcctatcgagaagaccatctctaaggccaagggccagcctagagagccacaggtgtacacactgcctccaagccgcgaggagatgaccaagaaccaggtgtctctgacatgtctggtgaagggcttctatccttctgacatcgctgtggagtgggagtccaatggccagccagagaacaattacaagaccacaccccctgtgctggactccgatggcagcttctttctgtattccaagctgaccgtggataagagcagatggcagcagggcaacgtgttttcttgctccgtgatgcatgaggctctgcacaatcattatacacagaagagcctgtctctgtcccctggcaagtga

SEQ ID NO: 19 - DNA, 720 bp, synthetic construct
  source 1..720: mol_type = other DNA
atggagttcggcctgtcctgggtgttcctggtggccctgtttagaggcgtgcagtgcgacgtggtcatgacacagtcccccctgagcctgcctgtgagcctgagacagcccgcttctatctcctgtcgctccagccagtctctggtgcattccgatggcaacacctacctgcattggtatctgcagaagccaggccagtctccccgcctgctgatctacaaggtgagcaatagggactctggcgtgcctgatcggttcagcggctctggctccggaaccgactttacactgaagatctccagggtggaggccgaggatctgggcgtgtatttctgcagccagtctacccactggccatggacatttggccagggcaccaagctggagatcaagcggacagtggcggcgccgtccgtgttcatctttccccctagcgacgagcagctgaagagcggcaccgcctctgtggtgtgcctgctgaacaatttctacccaagggaggccaaggtgcagtggaaggtggataacgctctgcagagcggcaattctcaggagtccgtgaccgagcaggacagcaaggattctacatattccctgtccagcaccctgacactgtctaaggccgactacgagaagcacaaggtgtatgcttgcgaggtgacccatcagggcctgtcttcccccgtgacaaagtcctttaaccggggcgagtgttgatga

SEQ ID NO: 20 - DNA, 363 bp, synthetic construct
  source 1..363: mol_type = other DNA
caggtgcagctggtgcagagcggagctgaggtggtgaagccaggagctagcgtgaagatgtcttgtaaggcctccggctacaccttcacatcttttctgatccactgggtgaagcaggctccaggacagggactggagtggatcggcttcatcatccctggcaacggcggcaccaagtacaatcagaagtttcagggcaaggccaccctgacatccgacacctccagctctacagcttatatggagctgtccagcctgaggtctgaggattccgccgtgtactattgcgctcggtacgacggcagcttcgagggctattttgacgtgtggggccagggcaccacactgacagtgtctagcgct

SEQ ID NO: 21 - DNA, 336 bp, synthetic construct
  source 1..336: mol_type = other DNA
gacgtggtcatgacacagtcccccctgagcctgcctgtgagcctgagacagcccgcttctatctcctgtcgctccagccagtctctggtgcattccgatggcaacacctacctgcattggtatctgcagaagccaggccagtctccccgcctgctgatctacaaggtgagcaatagggactctggcgtgcctgatcggttcagcggctctggctccggaaccgactttacactgaagatctccagggtggaggccgaggatctgggcgtgtatttctgcagccagtctacccactggccatggacatttggccagggcaccaagctggagatcaag

SEQ ID NO: 22 - DNA, 363 bp, synthetic construct
  source 1..363: mol_type = other DNA
gaggtccagctccagcagtctggacctgagctggtaaagcctggggcttcagtgaagatgtcctgcaaggcttctggatacacattcactagctttcttatacactgggtgaagcagaagcctgggcagggccttgagtggattggatttattattccttacaatgatggtactaagtacaatgagaagttcaaaggcaaggccacactgacttcagacaaatcctccagcacagcctacatggaactcagcagcctgacctctgaagactctgcggtctattactgtgcaagatatgatggtagcttcgagggttacttcgatgtctggggcgcagggaccactctcacagtctcctcggcc

SEQ ID NO: 23 - DNA, 336 bp, synthetic construct
  source 1..336: mol_type = other DNA
gacgttttgatgacccagactccactctccctgcctgtcagtcttagagatcaagcctccatctcttgcagatctagtcagagccttgtacacagtgatggaaacacctatttacattggtacctgcagaagccaggccagtctccaaagctcctgatctacaaagtttccaaccgattttcaggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 24 - DNA, 987 bp, synthetic construct
  source 1..987: mol_type = other DNA
tctaccaagggaccatccgtgttcccactggccccctccagcaagtccaccagcggaggaacagccgctctgggatgcctggtgaaggactacttcccagagcccgtgacagtgagctggaactctggcgctctgacctccggcgtgcacacatttccagctgtgctgcagtcttccggcctgtacagcctgagctctgtggtgaccgtgccctccagctctctgggcacccagacatatatctgcaacgtgaatcacaagccatccaatacaaaggtggacaagaaggtggagcccaagagctgtgataagacccatacatgccccccttgtcctgccccagagctgctgggaggaccatccgtgttcctgtttccacccaagcctaaggacaccctgatgatctctaggacccccgaggtgacatgcgtggtggtggacgtgtcccacgaggaccccgaggtgaagtttaactggtacgtggatggcgtggaggtgcataatgctaagaccaagcctagggaggagcagtacaacagcacctatcgggtggtgtctgtgctgacagtgctgcaccaggattggctgaacggcaaggagtataagtgcaaggtgagcaataaggccctgcccgctcctatcgagaagaccatctctaaggccaagggccagcctagagagccacaggtgtacacactgcctccaagccgcgaggagatgaccaagaaccaggtgtctctgacatgtctggtgaagggcttctatccttctgacatcgctgtggagtgggagtccaatggccagccagagaacaattacaagaccacaccccctgtgctggactccgatggcagcttctttctgtattccaagctgaccgtggataagagcagatggcagcagggcaacgtgttttcttgctccgtgatgcatgaggctctgcacaatcattatacacagaagagcctgtctctgtcccctggcaag

SEQ ID NO: 25 - DNA, 1350 bp, synthetic construct
  source 1..1350: mol_type = other DNA
gaggtccagctccagcagtctggacctgagctggtaaagcctggggcttcagtgaagatgtcctgcaaggcttctggatacacattcactagctttcttatacactgggtgaagcagaagcctgggcagggccttgagtggattggatttattattccttacaatgatggtactaagtacaatgagaagttcaaaggcaaggccacactgacttcagacaaatcctccagcacagcctacatggaactcagcagcctgacctctgaagactctgcggtctattactgtgcaagatatgatggtagcttcgagggttacttcgatgtctggggcgcagggaccactctcacagtctcctcggcctctaccaagggaccatccgtgttcccactggccccctccagcaagtccaccagcggaggaacagccgctctgggatgcctggtgaaggactacttcccagagcccgtgacagtgagctggaactctggcgctctgacctccggcgtgcacacatttccagctgtgctgcagtcttccggcctgtacagcctgagctctgtggtgaccgtgccctccagctctctgggcacccagacatatatctgcaacgtgaatcacaagccatccaatacaaaggtggacaagaaggtggagcccaagagctgtgataagacccatacatgccccccttgtcctgccccagagctgctgggaggaccatccgtgttcctgtttccacccaagcctaaggacaccctgatgatctctaggacccccgaggtgacatgcgtggtggtggacgtgtcccacgaggaccccgaggtgaagtttaactggtacgtggatggcgtggaggtgcataatgctaagaccaagcctagggaggagcagtacaacagcacctatcgggtggtgtctgtgctgacagtgctgcaccaggattggctgaacggcaaggagtataagtgcaaggtgagcaataaggccctgcccgctcctatcgagaagaccatctctaaggccaagggccagcctagagagccacaggtgtacacactgcctccaagccgcgaggagatgaccaagaaccaggtgtctctgacatgtctggtgaagggcttctatccttctgacatcgctgtggagtgggagtccaatggccagccagagaacaattacaagaccacaccccctgtgctggactccgatggcagcttctttctgtattccaagctgaccgtggataagagcagatggcagcagggcaacgtgttttcttgctccgtgatgcatgaggctctgcacaatcattatacacagaagagcctgtctctgtcccctggcaag

SEQ ID NO: 26 - DNA, 321 bp, synthetic construct
  source 1..321: mol_type = other DNA
cgtgaggtggcggcgccgtccgtgttcatctttccccctagcgacgagcagctgaagagcggcaccgcctctgtggtgtgcctgctgaacaatttctacccaagggaggccaaggtgcagtggaaggtggataacgctctgcagagcggcaattctcaggagtccgtgaccgagcaggacagcaaggattctacatattccctgtccagcaccctgacactgtctaaggccgactacgagaagcacaaggtgtatgcttgcgaggtgacccatcagggcctgtcttcccccgtgacaaagtcctttaaccggggcgagtgt

SEQ ID NO: 27 - DNA, 657 bp, synthetic construct
  source 1..657: mol_type = other DNA
gacgttttgatgacccagactccactctccctgcctgtcagtcttagagatcaagcctccatctcttgcagatctagtcagagccttgtacacagtgatggaaacacctatttacattggtacctgcagaagccaggccagtctccaaagctcctgatctacaaagtttccaaccgattttcaggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaacgtgaggtggcggcgccgtccgtgttcatctttccccctagcgacgagcagctgaagagcggcaccgcctctgtggtgtgcctgctgaacaatttctacccaagggaggccaaggtgcagtggaaggtggataacgctctgcagagcggcaattctcaggagtccgtgaccgagcaggacagcaaggattctacatattccctgtccagcaccctgacactgtctaaggccgactacgagaagcacaaggtgtatgcttgcgaggtgacccatcagggcctgtcttcccccgtgacaaagtcctttaaccggggcgagtgt

SEQ ID NO: 28 - DNA, 21 bp, synthetic construct
  source 1..21: mol_type = other DNA
acatcttttctgatccactgg

SEQ ID NO: 29 - DNA, 48 bp, synthetic construct
  source 1..48: mol_type = other DNA
ttcatcatccctggcaacggcggcaccaagtacaatcagaagtttcag

SEQ ID NO: 30 - DNA, 33 bp, synthetic construct
  source 1..33: mol_type = other DNA
tacgacggcagcttcgagggctattttgacgtg

SEQ ID NO: 31 - DNA, 45 bp, synthetic construct
  source 1..45: mol_type = other DNA
tccagccagtctctggtgcattccgatggcaacacctacctgcat

SEQ ID NO: 32 - DNA, 21 bp, synthetic construct
  source 1..21: mol_type = other DNA
aaggtgagcaatagggactct

SEQ ID NO: 33 - DNA, 27 bp, synthetic construct
  source 1..27: mol_type = other DNA
agccagtctacccactggccatggaca

SEQ ID NO: 34 - protein, 9 aa, synthetic construct
EEQYNSTYR

SEQ ID NO: 35 - protein, 26 aa, synthetic construct
THTCPPCPAPELLGGPSVFLFPPKPK

(19) United States
(12) Patent Application Publication
(10) Pub. No.: US 2026/0158153 A1
(43) Pub. Date: Jun. 11, 2026

(54) BCMA MONOCLONAL ANTIBODY AND THE ANTIBODY-DRUG CONJUGATE

(71) Applicant: HANGZHOU DAC BIOTECH CO., LTD., Hangzhou, Zhejiang (CN)

(72) Inventors: Robert Yongxin ZHAO, Lexington, MA (US); Junxiang JIA, Hangzhou, Zhejiang (CN); Yunxia ZHENG, Hangzhou, Zhejiang (CN); Qingliang YANG, Hangzhou, Zhejiang (CN); Yuanyuan HUANG, Hangzhou, Zhejiang (CN); Lingli ZHANG, Hangzhou, Zhejiang (CN); Wenjun LI, Hangzhou, Zhejiang (CN); Huihui GUO, Hangzhou, Zhejiang (CN); Hangbo YE, Hangzhou, Zhejiang (CN); Zhicang YE, Hangzhou, Zhejiang (CN); You ZHOU, Hangzhou, Zhejiang (CN); Juan WANG, Hangzhou, Zhejiang (CN)

(73) Assignee: HANGZHOU DAC BIOTECH CO., LTD., Hangzhou, Zhejiang (CN)

(21) Appl. No.: 18/706,736

(22) PCT Filed: Oct. 8, 2022
(86) PCT No.: PCT/CN2022/123901

§ 371 Date: May 1, 2024

(30) Foreign Application Priority Data
Nov. 3, 2021 (WO) PCT/CN2021/128453

(51) Int. Cl. A61K 47/68; A61K 47/54; A61P 35/00; C07K 16/28

(52) U.S. Cl. CPC A61K 47/68031; A61K 47/549; A61K 47/68033; A61K 47/68035; A61K 47/68037; A61K 47/6805; A61K 47/6809; A61K 47/6849; A61K 47/6889; A61P 35/00; C07K 16/2878; C07K 2317/565

(57) ABSTRACT

The disclosure is directed to an antibody and an antibody-drug conjugate (ADC) including a monoclonal antibody, or an antigen-binding fragment thereof, conjugated to a cytotoxin, directed against B-cell maturation antigen (BCMA). The BCMA antibody and its ADCs are useful for treatment of multiple myeloma cells, B-cell mediated or plasma cell mediated disease, and immune disorders as well as for detecting BCMA. Further disclosed are pharmaceutical compositions and medical treatment methods.

### CROSS-REFERENCE TO RELATED APPLICATIONS

This application is a Section 371 of International Application No. PCT/CN2022/123901, filed Oct. 8, 2022, which was published in the English language on May 11, 2023, under International Publication No. WO 2023/078021 A1, which claims priority under 35 U.S.C. § 119(b) to International Application No. PCT/CN2021/128453, filed Nov. 3, 2021, the disclosures of which are incorporated herein by reference in their entirety.

### REFERENCE TO SEQUENCE LISTING SUBMITTED ELECTRONICALLY

This application contains a sequence listing, which is submitted electronically as an XML formatted sequence listing with a file name “690167_2US_Sequence_Listing”, creation date of Apr. 26, 2024, and having a size of 40,420 bytes. The sequence listing submitted electronically is part of the specification and is herein incorporated by reference in its entirety.

### BACKGROUND

The B cell maturation antigen (BCMA, CD269) is a member of the tumor necrosis factor (TNF) receptor superfamily (Marino, S. F., et al, Data Brief. 2015, 6: 394-7. doi: 10.1016/j.dib.2015.12.023). BCMA binds to two distinct ligands, B cell activating factor (BAFF; also known as BlyS, TALL-1, TNFSF13B, and THANK) and a proliferation-inducing ligand (APRIL, TNFSF13) (Schiemann, B, et al, Science. 2001, 293(5537): 2111-4; Vidal-Laliena, M. et al, Cell Immunol. 236 (1-2): 6-16). The ligands for BCMA bind two additional TNF receptors, transmembrane activator and calcium modulator and cyclophilin ligand interactor (TACI) and BAFF receptor (BAFF-R also called BR3) (Yan, M. et al, Curr Biol. 2001, 11(19): 1547-52). Thus, BCMA is mostly known for its functional activity in mediating the survival of plasma cells that maintain long-term humoral immunity.

The expression of BCMA has been linked to a number of cancers, autoimmune disorders, and infectious diseases (Coquery, C. M. and Erickson, L. D. Crit. Rev. Immunol. 2012; 32(4): 287-305). BCMA protein is highly expressed on the surface of plasma cells from multiple myeloma patients (Novak et al, Blood, 103(2): 689-694 (2004); Neri et al., Clinical Cancer Research, 73(19): 5903-5909 (2007); and Moreaux et al., Blood, 703(8): 3148-3157 (2004)). As such, BCMA was extensively investigated as a therapeutic target for multiple myeloma and autoimmune disorders during the past decade (Ni, B. and Hou, J., Hematology. 2022, 27(1):343-352; Tan, C. R. and Shah, U. A., CurrHematolMalig Rep. 2021, 16(5): 367-383).

Multiple Myeloma (MM) is a frequent hematological malignancy worldwide (Sung, H, et al, CA Cancer J Clin. 2021, 71(3): 209-249). It is a hematologic malignancy characterized by proliferation of plasma cells with or without production of monoclonal immunoglobulins. Management of patients with MM typically begins with induction/neoadjuvant therapy (including chemotherapy, radiation, surgery, biophosphonates), typically a proteasome inhibitor (PI) with dexamethasone, an immunomodulator (IMID), followed by autologous (hematopoietic) stem cell transplantation (ASCT) in eligible patients. In the past five years, US FDA approved three monoclonal antibodies (daratumumab, isatuximab, elotuzumab), an exportin-1 inhibitor (selinexor), an anti-BCMA antibody-drug conjugate (belantamabmafodotin) and two BCMA chimeric antigen receptor (CAR)T-cell therapies (idecabtagenevicleucel and ciltacabtageneautoleucel) for the treatment of multiple myeloma. There are many ongoing clinical trials using novel targets and constructs, including bispecific antibodies targeting BCMA, GPRC5D, and FCRH5 of multiple myeloma. With the gradual improvement of treatment regimens, the survival time of multiple myeloma (MM) patients has been significantly prolonged. Even so, MM is still a nightmare with an inferior prognosis and the disease per se remains incurable (Davis, J. A. et al, J Oncol Pharm Pract. 2022 Jan. 10, doi: 10.1177/10781552211073517; Fuchsl, F. and Krackhardt, A. M. Cells. 2022 Jan. 25; 11(3), doi: 10.3390/cells11030410). And patients with progression after multiple treatment lines, including the use of monoclonal antibodies, have just a median overall survival of 8.6 months. Although the newly approved BCMA CAR-T cell therapies have demonstrated an overall efficacy of >80% in the clinical studies, they have their own set of limitations or challenges for manufacturing from patients' autologous cells, which result in extremely expensive treatment. Moreover, the first-in-class BCMA ADC, Belantamabmafodotin has only overall response rate of 32% for MM, plus over 60% patients with this ADC reported peculiar side effects of several ocular toxicities requiring dose adjustments, dose delays and treatment discontinuations (Wahab, A. et al, Front Oncol. 2021, 11:678634. doi: 10.3389/fonc.2021.678634). Thus, a more MM treatment options in feasibility, safety, and promising efficacy, in particular, a therapy to overcome the resistance of existing drugs are still urgently needed.

Here in this patent application, we disclose a BCMA antibody and an antibody-drug conjugate (ADC) comprising a BCMA monoclonal antibody, or a BCMA antigen-binding fragment thereof, conjugated to a cytotoxin, directed against B-cell maturation antigen (BCMA). The BCMA antibodies and its ADCs are useful for treatment and diagnoses of various cancers, autoimmune disorders, and infectious diseases as well as detecting BCMA. This invention also continues to apply the methodology of specific conjugation (PCT/CN2021/128453) to construct these BCMA ADCs. Further disclosed are pharmaceutical compositions, screening and medical treatment methods.

### BRIEF SUMMARY OF THE INVENTION

The present invention provides antigen binding proteins which specifically bind to BCMA (CD269), for example antibodies which specifically bind to BCMA and which inhibit the binding of BAFF and/or APRIL to the BCMA receptor. The present invention also provides antigen binding proteins which specifically bind to BCMA and which inhibits the binding of BAFF and/or APRIL to BCMA and wherein the antigen binding protein is capable of internalization. The BCMA monoclonal antibody comprises (a) a heavy chain variable region comprising a complementarity determining region 1 (HCDR1) amino acid sequence of SEQ ID NO: 1, an HCDR2 amino acid sequence of SEQ ID NO: 2, and an HCDR3 amino acid sequence of SEQ ID NO: 3 and (b) a light chain variable region comprising a complementarity determining region 1 (LCDR1) amino acid sequence of SEQ ID NO: 4, an LCDR2 amino acid sequence of SEQ ID NO: 5, and an LCDR3 amino acid sequence of SEQ ID NO: 6.

The present invention also provides an antibody-drug conjugate (ADC) comprising a monoclonal antibody, or an antigen-binding fragment thereof, directed against B-cell maturation antigen (BCMA) conjugated to a cytotoxin. In a further embodiment the antigen binding proteins are conjugated to a toxin such as a tubulysin analog, a PBD dimer or an auristatin analog.

In addition, the invention provides compositions comprising the foregoing antibody-drug conjugate, and a pharmaceutically acceptable carrier, and methods of killing multiple myeloma cells (including multiple myeloma stem cells) that express BCMA by contacting multiple myeloma cells with the ADC.

In another aspect of the present invention there is provided a method of treating a human patient afflicted with a B cell related disorders or diseases such as antibody mediated or plasma cell mediated diseases or plasma cell malignancies such as for example Multiple Myeloma (MM) which method comprises the step of administering to said patient a therapeutically effective amount of the antigen binding antibody and/or ADC thereof as described herein.

In a further aspect of the present invention there is provided a method of treating a human patient afflicted with Rheumatoid Arthritis, Psoriasis, Type 1 Diabetes Mellitus or Multiple Sclerosis which method comprises the step of administering to said patient a therapeutically effective amount of the antigen binding protein and/or ADC as described herein.

### BRIEF DESCRIPTION OF THE SEVERAL VIEWS OF THE DRAWING(S)

FIG. 1 shows binding affinity of hybridoma antibody BCMA-A2-6H4-5D2 and positive control antibody J6M0 to recombinant expressed TrxA-BCMA.

FIG. 2 shows Binding affinity of hybridoma antibody BCMA-A2-6H4-5D2, chimeric antibody c5D2, humanized antibody hu5D2 and positive control antibody J6M0 to recombinant expressed TrxA-BCMA.

FIG. 3 shows Binding affinity of humanized antibody hu5D2, hu5D2 conjugated ADC and isotype control antibody to endogenous BCMA expressed cell line NCI-H929.

FIG. 4A illustrates the killing of BCMA over-expressed RPMI-8226 cell lines by the antibody drug conjugate, hu5D2-tubulysin B analog conjugate (C-390).

FIG. 4B illustrates the killing of cell line NCI-H929 by the antibody drug conjugate: hu5D2-tubulysin B analog conjugate (C-390), in comparison to the ADC J6M0-tubulysin B analog conjugate (C-390), naked hu5D2 antibody, unconjugated tubulysin B analog (compound 390) and Paclitaxel.

FIG. 4C illustrates the killing of cell line MNM. S by the antibody drug conjugate: hu5D2-tubulysin B analog conjugate (C-390), in comparison with the ADC J6M0-tubulysin B analog conjugate (C-390), naked hu5D2 antibody, unconjugated tubulysin B analog (compound 390) and Paclitaxel.

FIG. 4D illustrates the killing of BCMA negative expression cell line Jurkat by the antibody drug conjugate hu5D2-tubulysin B analog conjugate (C-390), in comparison with the ADC J6M0-tubulysin B analog conjugate (C-390), naked hu5D2 antibody, unconjugated tubulysin B analog (compound 390) and Paclitaxel.

FIG. 4E illustrates the killing of BCMA expression cell line U266B1 by the BCMA antibody (hu5D2)-drug conjugates: C-221, C-202, C-88, C-326, C-30, in comparison with Paclitaxel.

FIGS. 5(a)-(h) illustrate MS/MS daughter or product ion spectrum of glycopeptides of the BCMA antibody. (a): Non-glycosylated peptides; (b): Man5 containing glycopeptides; (c): G0F-GlcNAc containing glycopeptides; (d): G0 containing glycopeptides; €: G0F containing glycopeptides; (f): G1 containing glycopeptides; (g): G1F containing glycopeptides; (h): G2F containing glycopeptides.

FIG. 6 illustrates middle-level characterization of BCMA-Tubulysin Banalog ADC (C-390) after N-deglycosylation and reduction. (a) rpHPLC chromatogram of ADC fragments obtained after deglycosylation and DTT reduction. Light chains (LC) with zero or one drug molecule attached (L0 and L1), (b) heavy chains with zero, one, two, or three drug molecules attached (H0, H1, H2 and H3).

FIG. 7 illustrates the Percentage of Drug Loaded Peptides of a BCMA ADC (C-390). (a): LC Peptide [GEC] with zero or one drug molecule attached (D0 and D1); (b): HC Peptide [SCDK] at the arm with zero or one drug molecule attached (D0 and D1); (c): HC Peptide [THTCPPCPAPELLGGPSVFLFPPKPK] at the hinge with zero, one or two drug molecules attached (D0, D1 and D2).

FIG. 8 illustrates MS/MS daughter or product ion spectrum of drug-loaded peptides of a BCMA-ADC (C-390).
(a): Heavy chain [SC₍₂₂₃₎DK]+1 Drug;
(b): Heavy chain [THTC₍₂₂₉₎PPCPAPELLGGPSVFLFPPKPK]+1 Drug;
(c): Heavy chain [THTCPPC₍₂₃₂₎PAPELLGGPSVFLFPPKPK]+1 Drug;
(d): Heavy chain [THTC₍₂₂₉₎PPC₍₂₃₂₎PAPELLGGPSVFLFPPKPK]+2 Drug;
(e): Light chain [GEC₍₂₁₉₎]+1 Drug.

FIG. 9 illustrates change in tumor volume in a NCI-H929 cell xenograft mouse model of multiple myeloma in response to a serial of single dose (3 mg/Kg) treatment with BCMA (hu5D2) ADCs (C-68a, C-115, C-192, C-202, C-221, C-290, C-306, C-385, C-390, C-399, C-402, C-417, DARs indicated in table 7), in comparison to BCMA-mcMMAF (belantamabmcMMAF) and PBS buffer (the control). The figure indicates that all the 13 conjugates had antitumor activity, and the orders of the antitumor activity are: C-385<C-306<C-290<C-68a<C-115<BCMA-mcMMAF<C-192<C-202<C-399<C-390<C-417<C-402<C-221.

FIG. 10 illustrates change in tumor volume in a JJN-3 cell xenograft mouse model of multiple myeloma in response to a serial of single dose (5 mg/Kg) treatment with hu5D2-ADC in comparison to belantamabmcMMAF and PBS buffer (the control). The figure indicates that all the 9 conjugates had antitumor activity, and the orders of the antitumor activity are: Paclitaxel<C-385<belantamabmcMMAF<C-195<C-137<C-181b<C-126<C-83<C-277<C-258.

FIG. 11 illustrates change in tumor volume in NCI-H929 cell xenograft mouse model of multiple myeloma in response to a serial of single dose (2 mg/Kg) treatment with hu5D2-ADC in comparison to belantamabmcMMAF and PBS buffer (the control). The figure indicates that all the 7 conjugates had antitumor activity, and the orders of the antitumor activity are: belantamabmcMMAF<C-406<C-396<C-399<C-400<C-221b<C-402.

FIG. 12 shows the general synthesis of components of a bis-linker.

FIG. 13 shows the synthesis of a camptothecin analog containing a bis-conjugate linker.

FIG. 14 shows the synthesis of a camptothecin analog containing a bis-conjugate linker.

FIG. 15 shows the synthesis of a camptothecin analog containing a bis-conjugate linker.

FIG. 16 shows the general synthesis of components of a bis-linker.

FIG. 17 shows the synthesis of a camptothecin analog containing a bis-conjugate linker.

FIG. 18 shows the synthesis of a camptothecin analog containing a bis-conjugate linker.

FIG. 19 shows the synthesis of a camptothecin analog containing a bis-conjugate linker.

FIG. 20 shows the general synthesis of components of a bis-linker.

FIG. 21 shows the synthesis of a camptothecin analog containing a bis-conjugate linker.

FIG. 22 shows the synthesis of a tubulysin B analog containing a bis-conjugate linker.

FIG. 23 shows the synthesis of a tubulysin B analog containing a bis-conjugate linker.

FIG. 24 shows the synthesis of a tubulysin B analog containing a bis-conjugate linker.

FIG. 25 shows the synthesis of a tubulysin B analog containing a bis-conjugate linker.

FIG. 26 shows the synthesis of a tubulysin B analog containing a bis-conjugate linker.

FIG. 27 shows the synthesis of a tubulysin B analog containing a bis-conjugate linker.

FIG. 28 shows the synthesis of components of a tubulysin B analogs.

FIG. 29 shows the synthesis of a tubulysin B analog containing a bis-conjugate linker.

FIG. 30 shows the synthesis of a tubulysin B analog containing a bis-conjugate linker.

FIG. 31 shows the synthesis of a camptothecin analog containing a bis-conjugate linker.

FIG. 32 shows the synthesis of a camptothecin analog containing a bis-conjugate linker.

FIG. 33 shows the synthesis of a camptothecin analog containing a bis-conjugate linker.

FIG. 34 shows the synthesis of a camptothecin analog containing a bis-conjugate linker.

FIG. 35 shows the synthesis of a camptothecin analog containing a bis-conjugate linker.

FIG. 36 shows the synthesis of a camptothecin analog containing a bis-conjugate linker and components of amanitin analogs.

FIG. 37 shows the synthesis of an amanitin analog containing a bis-conjugate linker.

FIG. 38 shows the synthesis of an amanitin analog containing a bis-conjugate linker.

FIG. 39 shows the synthesis of an amanitin analog containing a bis-conjugate linker.

FIG. 40 shows the synthesis of an amanitin analog containing a bis-conjugate linker.

FIG. 41 shows the synthesis of a tubulysin B analog containing a linker.

FIG. 42 shows the synthesis of a tubulysin B analog containing a linker.

FIG. 43 shows the synthesis of a tubulysin B analog containing a linker.

FIG. 44 shows the synthesis of a tubulysin B analog containing a linker.

FIG. 45 shows the synthesis of a tubulysin B analog containing a linker.

FIG. 46 shows the synthesis of a tubulysin B analog containing a linker.

FIG. 47 shows the synthesis of a tubulysin B analog containing a linker.

FIG. 48 shows the synthesis of a tubulysin B analog containing a linker.

FIG. 49 shows the synthesis of a tubulysin B analog containing a linker.

FIG. 50 shows the synthesis of a tubulysin B analog containing a linker.

FIG. 51 shows the synthesis of a tubulysin B analog containing a linker.

FIG. 52 shows the synthesis of a tubulysin B analog containing a linker.

FIG. 53 shows the synthesis of a tubulysin B analog containing a linker.

FIG. 54 shows the synthesis of a tubulysin B analog containing a linker.

FIG. 55 shows the synthesis of a tubulysin B analog containing a linker.

FIG. 56 shows the synthesis of a tubulysin B analog containing a linker and components of PBD analogs.

FIG. 57 shows the synthesis of a PBD analog containing a linker.

FIG. 58 shows the synthesis of a PBD analog containing a linker.

FIG. 59 shows the synthesis of a PBD analog containing a linker and a tubulysin B analog containing a linker.

FIG. 60 shows the synthesis of a tubulysin B analog containing a linker.

FIG. 61 shows the synthesis of a tubulysin B analog containing a linker.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2, 2-dimethylbutyl, 2, 3-dimethylbutyl, 2, 2-dimethylpentyl, 2, 3-dimethylpentyl, 3, 3-dimethylpentyl, 2, 3, 4-trimethylpentyl, 3-methyl-hexyl, 2, 2-dimethylhexyl, 2, 4-dimethylhexyl, 2, 5-dimethylhexyl, 3, 5-dimethylhexyl, 2, 4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4, 5], [5, 5], [5, 6] or [6, 6] system, or 9 or 10 ring atoms arranged as a bicycle [5, 6] or [6, 6] system. Representative C₃-C₈ carbocycles include, but are not limited to, -cyclopropyl, -cyclobutyl, -cyclopentyl, -cyclopentadienyl, -cyclohexyl, -cyclohexenyl, -1, 3-cyclohexadienyl, -1, 4-cyclohexadienyl, -cycloheptyl, -1, 3-cycloheptadienyl, -1, 3, 5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1, 2-ethyl (—CH₂CH₂—), 1, 3-propyl (—CH₂CH₂CH₂—), 1, 4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1, 2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1, 2, 4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoro-methylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1, 1′-carbonyldiimidazole; DCC, dicyclohexylcarbodiimide; DCE, dichloroethane; DCM, dichloromethane; DIAD, diisopropylazodicarboxylate; DIBAL-H, diisobutyl-aluminium hydride; DIPEA, diisopropylethylamine; DEPC, diethyl phosphorocyanidate; DMA, N, N-dimethyl acetamide; DMAP, 4-(N, N-dimethylamino)pyridine; DMF, N, N-dimethylformamide; DMSO, dimethylsulfoxide; DTT, dithiothreitol; EDC, 1-(3-dimethylaminopropyl)-3-ethylcarbodiimide hydrochloride; ESI-MS, electrospray mass spectrometry; HATU, O-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuc-cinimide; MMP, 4-methylmorpholine; PAB, p-aminobenzyl; PBS, phosphate-buffered saline (pH 7.0˜7.5); PEG, polyethylene glycol; SEC, size-exclusion chromatography; TCEP, tris(2-carboxyethyl)phosphine; TFA, trifluoroacetic acid; THF, tetrahydrofuran; Val, valine.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3, 4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1˜8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Val-Ala-Val, Lys-Lys, Ala-Asn-Val, Val-Leu-Lys, Cit-Cit, Val-Lys, Ala-Ala-Asn, Lys, Cit, Ser, and Glu.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, PA, 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

The abbreviations of biological buffers and their chemical names are listed below:
ACES (N-(2-Acetamido)-2-aminoethanesulfonic acid) is used to buffer at pH 6.1-7.5 (pKa=6.88)
ADA (N-(2-Acetamido)iminodiacetic acid, N-(Carbamoylmethyl)iminodiacetic acid) is useful to buffer at pH 6.0-7.2 (pKa=6.65).
AMPD (2-amino-2-methyl-1, 3-propanediol)) is a useful buffer at pH 7.8-9.7.
AMPSO (N-(1, 1-Dimethyl-2-hydroxyethyl)-3-amino-2-hydroxypropanesulfonic acid).
BES (N, N-Bis(2-hydroxyethyl)-2-aminoethanesulfonic acid).
Bicine (N, N-Bis(2-hydroxyethyl)glycine], Bis(2-hydroxyethyl)amino-tris(hydroxymethyl) methane) is used to buffer at pH 5.8-7.2 (pKa=8.35).
BisTris (Bis-(2-Hydroxyethyl)amino-tris(Hydroxymethyl)Methane).
BisTris propane (1, 3-Bis[tris(hydroxymethyl)methylamino]propane).
DIPSO (N, N-Bis(2-hydroxyethyl)-3-amino-2-hydroxypropanesulfonic acid) is used to buffer at pH 7.0-8.2.
Gly-Gly (Diglycine; Glycyl-glycine) is used to buffer at pH 7.5-8.9 (pKa=8.30).
HEBPS (N-(2-Hydroxyethyl)piperazine-N′-(4-butanesulfonic acid)) is an homolog of HEPES and EPPS with higher pKa (pKa=8.30), used to buffer at pH 7.6-9.0
HEPES (4-(2-hydroxyethyl)-1-piperazineethanesulfonic acid; 2-morpholinoethanesulfonic acid; 2-(4-morpholino)ethanesulphonic acid; 2-(N-morpholino)ethanesulfonic acid; morpholine-4-ethanesulfonic acid hydrate) is widely used to buffer at pH 6.8-8.2; pKa at 20° C.: 7.45-7.65)
HEPPS or EPPS (3-[4-(2-Hydroxyethyl)-1-piperazinyl]propanesulfonic acid hydrate; 4-(2-Hydroxyethyl)piperazine-1-(2-hydroxypropanesulfonic acid) Hydrate) is used as a buffering agent at pH 7.3-8.7 (pKa=8.00/piperazine ring).
HEPPSO (4-(2-Hydroxyethyl)piperazine-1-(2-hydroxypropanesulfonic acid) hydrate).
MES (2-(N-morpholino)ethanesulfonic acid, monohydrate) is used as buffering agent at pH 5.2-7.1 (pKa:6.16).
MOBS (4-Morpholinebutanesulfonic acid; 3-(N-Morpholino)butanesulfonic acid hemisodium salt) is an homolog of MES and MOPS with higher pKa/It is used to buffer solution at pH6.9-8.3 (pKa:7.6).
MOPS (4-Morpholinepropanesulfonic acid sodium salt).
MOPSO (β-Hydroxy-4-morpholinepropanesulfonic acid, 3-Morpholino-2-hydroxypropanesulfonic acid).
PIPES (Piperazine-1, 4-bis(2-ethanesulfonic acid) is used to buffer at pH 6.1-7.5 (pKa=6.80).
POPSO (Piperazine-1, 4-bis(2-hydroxypropanesulfonic acid) dihydrate).
TAPS ([(2-Hydroxy-1, 1-bis(hydroxymethyl)ethyl)amino]-1-propanesulfonic acid).
TAPSO (2-Hydroxy-3-[tris(hydroxymethyl)methylamino]-1-propanesulfonic acid).
TES (2-[(2-Hydroxy-1, 1-bis(hydroxymethyl)ethyl)amino]ethanesulfonic acid).
Tricine (Piperazine-N, N′-Bis[2-Hydroxypropanesulfonic Acid)] is used to buffer at pH7.4-8.8 (pKa:8.16).

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150: 880-887 (1993); Clarkson et al., Nature 352: 624-628 (1991).

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495, 1975, or may be made by recombinant DNA methods such as described in U.S. Pat. No. 4,816,567. The monoclonal antibodies may also be isolated from phage libraries generated using the techniques described in McCafferty et al., Nature 348:552-554, 1990, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen binding subsequences of antibodies) that contain minimal sequence derived from non-human immunoglobulin. Preferably, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “monovalent antibody” comprises one antigen binding site per molecule (e.g., IgG or Fab). In some instances, a monovalent antibody can have more than one antigen binding sites, but the binding sites are from different antigens.

A “monospecific antibody” comprises two identical antigen binding sites per molecule (e.g. IgG) such that the two binding sites bind identical epitope on the antigen. Thus, they compete with each other on binding to one antigen molecule. Most antibodies found in nature are monospecific. In some instances, a monospecific antibody can also be a monovalent antibody (e.g. Fab).

A “bivalent antibody” comprises two antigen binding sites per molecule (e.g., IgG). In some instances, the two binding sites have the same antigen specificities. However, bivalent antibodies may be bispecific.

A “bispecific” or “dual-specific” is a hybrid antibody having two different antigen binding sites. The two antigen binding sites of a bispecific antibody bind to two different epitopes, which may reside on the same or different protein targets.

A “bifunctional” is antibody is an antibody having identical antigen binding sites (i.e., identical amino acid sequences) in the two arms but each binding site can recognize two different antigens.

A “heteromultimer”, “heteromultimeric complex”, or “heteromultimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present.

A “heterodimer”, “heterodimeric protein”, “heterodimeric complex,” or “heteromultimeric polypeptide” is a molecule comprising a first polypeptide and a second polypeptide, wherein the second polypeptide differs in amino acid sequence from the first polypeptide by at least one amino acid residue.

The “hinge region”, “hinge sequence”, and variations thereof, as used herein, includes the meaning known in the art, which is illustrated in, for example, Janeway et al., ImmunoBiology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

The “immunoglobulin-like hinge region”, “immunoglobulin-like hinge sequence,” and variations thereof, as used herein, refer to the hinge region and hinge sequence of an immunoglobulin-like or an antibody-like molecule (e.g., immunoadhesins). In some embodiments, the immunoglobulin-like hinge region can be from or derived from any IgG1, IgG2, IgG3, or IgG4 subtype, or from IgA, IgE, IgD or IgM, including chimeric forms thereof, e.g., a chimeric IgG1/2 hinge region.

The term “immune effector cell” or “effector cell” as used herein refers to a cell within the natural repertoire of cells in the human immune system which can be activated to affect the viability of a target cell. The viability of a target cell can include cell survival, proliferation, and/or ability to interact with other cells.

Antibodies of the invention can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4): 924-40, 2007).

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, In111, Re186, Re188, Sm153, Bi212, P32, Pb212, Zr89, F18, and radioactive isotopes of Lu, e.g. Lu177); chemotherapeutic agents or drugs (e.g., tubulysin, maytansin, auristatin, DNA minor groove binders (such as PBD dimers), ducarmysin, topoisomerase inhibitor, RNA polymerase inhibitors, DNA alkylators, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof, and the various antitumor or anticancer agents disclosed throughout the application.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)nO(CR₂) n--, repeating units of alkyloxy (e.g. polyethylenoxy, PEG, polymethyleneoxy) and alkylamino (e.g. polyethyleneamino); and diacid ester and amides including succinate, succinamide, diglycolate, malonate, and caproamide. In various embodiments, linkers can comprise one or more amino acid residues, such as valine, phenylalanine, lysine, and homolysine.

The words “comprise”, “comprising”, “include”, “including” and “includes” when used in this specification and claims are intended to specify the presence of stated features, integers, components, or steps, but they do not preclude the presence or addition of one or more other features, integers, components, steps, or groups thereof. The novel conjugates disclosed herein are BSMA antibody conjugates. Examples of the conjugates and their synthesis are shown in the examples 9-379 below.

### BCMA Antibody and its Antibody Drug Conjugate.

The invention provides monoclonal antibodies that specifically bind to BCMA (CD269). Unless otherwise indicated, BCMA means a human BCMA. Exemplary human nucleic acid and amino acid sequences are provided by SEQ ID Nos:1 and 2. Unless otherwise apparent from the context reference to BCMA means at least an extracellular domain of the protein (approximately residues 1-54 of SEQ ID NO: 7) and sometimes the complete protein.

The present invention provides a method for the treatment of a medical disorder in a human subject, wherein the medical disorder is associated with the presence of pathogenic B cells expressing B cell maturation antigen (BCMA), the method comprising administering to the human subject an isolated monoclonal antibody or an antigen binding fragment thereof that binds BCMA (CD269).

The present BCMA antibody (e. q.hu5D2) is a humanized monoclonal antibody that specifically binds to human BCMA as described in the examples. The 5D2 antibody was produced by hybridoma BCMA-A2-6H4-5D2. A deposit at China Center for Type Culture Collection (CCTCC) was made on Jun. 23, 2022 under the Budapest Treaty. The CCTCC is located at Wuhan University, Wuhan City, Hubei, Post code 430000, P. R. China. The CCTCC deposit was assigned accession number of CCTCC C2022188. Hu5D2antibody inhibits binding of BCMA to both of its ligands, APRIL and BAFF. The Hu5D2antibody when linked to a human IgG1 elicits ADCC, binds to and elicits signaling through Fcγ. receptors. The Hu5D2antibody can also be incorporated into an antibody drug conjugate to deliver a linked drug into the interior of cells expressing BCMA.

The Hu5D2antibody is another humanized monoclonal antibody that specifically binds to human BCMA, inhibits its binding to its ligands and can deliver a linked drug to the interior of cells expressing BCMA.

The present invention provides antigen binding proteins which bind to membrane bound targets and wherein the antigen binding protein is capable of internalisation. In a further embodiment there is provided an immunoconjugate comprising the antigen binding protein of the present invention and a cytotoxic agent. In a further embodiment the antigen binding protein has ADCC effector function for example the antigen binding protein has enhanced ADCC effector function.

In one such embodiment there is provided antigen binding proteins or fragments thereof which specifically bind to BCMA, for example which specifically binds human BCMA (hBCMA) and which inhibit the binding of BAFF and/or APRIL to the BCMA receptor.

In a further embodiment the antigen binding proteins or fragments of the present invention specifically bind to BCMA and inhibit the binding of BAFF and/or APRIL to BCMA wherein the antigen binding proteins or fragments thereof have the ability to bind to FcγRIIIA and mediate FcgRIIIA mediated effector functions, or have enhanced Fc.γRIIIA mediated effector function. In one embodiment of the invention as herein provided the antigen binding proteins are capable of internalisation.

In one aspect of the invention there is provided an antigen binding protein according to the invention as herein described which binds to non-membrane bound BCMA, for example to serum BCMA.

In one aspect of the invention there is provided an antigen binding protein as herein described wherein the antigen binding protein comprises CDRH3 of SEQ ID NO. 3 or a variant of SEQ ID NO. 3.

In a further aspect of the invention there is provided an antigen binding protein as herein described wherein the antigen binding protein further comprises one or more of: CDR H1 of SEQ. ID. NO: 1, CDRH2: SEQ. ID. NO: 2: CDRL1: SEQ. ID. NO: 4, CDRL2: SEQ. ID. NO: 5 and/or CDRL3: SEQ. ID. NO: 6 and or variants thereof.

The antigen binding proteins of the present invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full length antibody, a (Fab′)2 fragment, a Fab fragment, or equivalent thereof (such as scFV, bi- tri- or tetra-bodies, Tandabs etc.), when paired with an appropriate light chain. The antibody may be an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. Furthermore, the antigen binding protein may comprise modifications of all classes e.g. IgG dimers, Fc mutants that no longer bind Fc receptors or mediate C1q binding. The antigen binding protein may also be a chimeric antibody of the type described in WO86/001533 which comprises an antigen binding region and a non-immunoglobulin region.

The constant region is selected according to any functionality required e.g. an IgG1 may demonstrate lytic ability through binding to complement and/or will mediate ADCC (antibody dependent cell cytotoxicity).

The antigen binding proteins of the present invention are derived from the murine antibody having the variable regions as described in SEQ ID NO: 10 and SEQ ID NO: 11 or non-murine equivalents thereof, such as rat, human, chimeric or humanised variants thereof, for example they are derived from the antibody having the variable heavy chain sequences as described in SEQ ID NO:10, and/or the variable light chain sequences as described in SEQ ID NO: 11.

In one aspect of the invention there is provided an antigen binding protein comprising an isolated heavy chain variable domain selected from any one of the following: SEQ ID NO: 8, SEQ ID NO:10, or SEQ ID NO:13.

In another aspect of the invention there is provided an antigen binding protein comprising an isolated light chain variable domain selected from any one of the following: SEQ ID NO:9, SEQ ID NO:11 or SEQ ID NO:15.

In a further aspect of the invention there is provided an antigen binding protein comprising an isolated heavy chain variable domain selected from any one of the following: SEQ ID NO: 8, SEQ ID NO:10, and SEQ ID NO:13 and an isolated light chain variable domain selected from any one of the following: SEQ ID NO:9, SEQ ID NO:11 and/or SEQ ID NO:15.

In one aspect the antigen binding protein of the present invention comprises a heavy chain variable region encoded by SEQ. ID. NO:20 or SEQ. ID. NO:22 and a light chain variable region encoded by SEQ. ID. NO:21 or SEQ. ID. NO:23

In one aspect there is provided a polynucleotide encoding an isolated variable heavy chain said polynucleotide comprising SEQ. ID. NO. 28, or SEQ. ID. NO. 29, or SEQ. ID. NO. 30.

In one aspect there is provided a polynucleotide encoding an isolated variable light chain said polynucleotide comprising SEQ. ID. NO. 31, or SEQ. ID. NO. 32, or SEQ. ID. NO. 33.

In a further aspect the antigen binding protein may comprise any one of the variable heavy chains as described herein in combination with any one of the light chains as described herein.

In one aspect the antigen binding protein is an antibody or antigen binding fragment thereof comprising one or more CDR's according to the invention described herein, or one or both of the heavy or light chain variable domains according to the invention described herein. In one embodiment the antigen binding protein binds primate BCMA. In one such embodiment the antigen binding protein additionally binds non-human primate BCMA, for example cynomolgus macaque monkey BCMA.

In another aspect the antigen binding protein is selected from the group consisting of a dAb, Fab, Fab′, F(ab′)₂, Fv, diabody, triabody, tetrabody, miniantibody, and a minibody.

In one aspect of the present invention the antigen binding protein is a humanised or chimaeric antibody, in a further aspect the antibody is humanised. In one aspect the antibody is a monoclonal antibody.

In another aspect the antigen binding protein binds to human BCMA with high affinity for example when measured by Biacore the antigen binding protein binds to human BCMA with an affinity of 20 nM or less or an affinity of 15 nM or less or an affinity of 5 nM or less or an affinity of 1000 pM or less or an affinity of 500 pM or less or an affinity of 400 pM or less, or 300 pM or less or for example about 120 pM. In a further embodiment the antigen binding protein binds to human BCMA when measured by Biacore of between about 100 pM and about 500 pM or between about 100 pM and about 400 pM, or between about 100 pM and about 300 pM. In one embodiment of the present invention the antigen binding protein binds BCMA with an affinity of less than 150 pm.

In one such embodiment, this is measured by Biacore, for example as set out in Example 4.

In another aspect the antigen binding protein binds to human BCMA and neutralises the binding of the ligands BAFF and/or APRIL to the BCMA receptor in a cell neutralisation assay wherein the antigen binding protein has an 1050 of between about 1 nM and about 500 nM, or between about 1 nM and about 100 nM, or between about 1 nM and about 50 nM, or between about 1 nM and about 25 nM, or between about 5 nM and about 15 nM. In a further embodiment of the present invention the antigen binding protein binds BCMA and neutralises BCMA in a cell neutralisation assay wherein the antigen binding protein has an 1050 of about 10 nM.

The antigen binding proteins, for example antibodies of the present invention may be produced by transfection of a host cell with an expression vector comprising the coding sequence for the antigen binding protein of the invention. An expression vector or recombinant plasmid is produced by placing these coding sequences for the antigen binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen binding protein light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen binding protein may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen binding protein of the invention. The antigen binding protein which includes the association of both the recombinant heavy chain and/or light chain is screened from culture by appropriate assay, such as ELISA or RIA. Similar conventional techniques may be employed to construct other antigen binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as AmershamBioscience (Buckinghamshire, United Kingdom) or GenScript (Nanjing, China). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen binding proteins of the present invention. Host cells useful for the cloning and other manipulations of these cloning vectors are also conventional. However, cells from various strains of E. coli may be used for replication of the cloning vectors and other steps in the construction of antigen binding proteins of this invention.

Suitable host cells or cell lines for the expression of the antigen binding proteins of the invention include mammalian cells such as NS0, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns.

Alternatively, other eukaryotic cell lines may be employed. The selection of suitable mammalian host cells and methods for transformation, culture, amplification, screening and product production and purification are known in the art. See, e.g., Sambrook et al., (1989). Molecular cloning: a laboratory manual, 2nd ed. Cold Spring Harbor Laboratory, Cold Spring Harbor, N.Y.

Bacterial cells may prove useful as host cells suitable for the expression of the recombinant Fabs or other embodiments of the present invention (see, e.g., Pluckthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. Coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and McGuire, S. et al, Trends Genet. (2004) 20, 384-391 and references cited therein.

The general methods by which the vectors may be constructed, the transfection methods required to produce the host cells of the invention, and culture methods necessary to produce the antigen binding protein of the invention from such host cell may all be conventional techniques. Typically, the culture method of the present invention is a serum-free culture method, usually by culturing cells serum-free in suspension. Likewise, once produced, the antigen binding proteins of the invention may be purified from the cell culture contents according to standard procedures of the art, including ammonium precipitation, affinity columns, column chromatography, gel electrophoresis and the like. Such techniques are within the skill of the art and do not limit this invention. For example, preparations of altered antibodies are described in WO 99/058679 and WO 96/016990. Yet another method of expression of the antigen binding proteins may utilize expression in a transgenic animal, such as described in U.S. Pat. No. 4,873,316. This relates to an expression system using the animals casein promoter which when transgenically incorporated into a mammal permits the female to produce the desired recombinant protein in its milk.

In a further embodiment of the invention there is provided a method of producing an antibody of the invention which method comprises the step of culturing a host cell transformed or transfected with a vector encoding the light and/or heavy chain of the antibody of the invention and recovering the antibody thereby produced.

In accordance with the present invention there is provided a method of producing an anti-BCMA antibody of the present invention which binds to and neutralises the activity of human BCMA which method comprises the steps of, providing a first vector encoding a heavy chain of the antibody; providing a second vector encoding a light chain of the antibody; transforming a mammalian host cell (e.g. CHO) with said first and second vectors; culturing the host cell of step (c) under conditions conducive to the secretion of the antibody from said host cell into said culture media; recovering the secreted antibody of step (d).

Once expressed by the desired method, the antibody is then examined for in vitro activity by use of an appropriate assay. Presently conventional ELISA assay formats are employed to assess qualitative and quantitative binding of the antibody to BCMA. Additionally, other in vitro assays may also be used to verify neutralizing efficacy prior to subsequent human clinical studies performed to evaluate the persistence of the antibody in the body despite the usual clearance mechanisms.

The dose and duration of treatment relates to the relative duration of the molecules (the antibody and the antibody-drug conjugate) of the present invention in the human circulation, and can be adjusted by one of skill in the art depending upon the condition being treated and the general health of the patient. It is envisaged that repeated dosing (e.g. once a week or once every two weeks or once every 3 weeks or once every 4 weeks) over an extended time period (e.g. four to six months) may be required to achieve maximal therapeutic efficacy.

In one embodiment of the present invention there is provided a recombinant transformed, transfected or transduced host cell comprising at least one expression cassette, for example where the expression cassette comprises a polynucleotide encoding a heavy chain of an antigen binding protein according to the invention described herein and further comprises a polynucleotide encoding a light chain of an antigen binding protein according to the invention described herein or where there are two expression cassettes and the 1.sup.st encodes the light chain and the second encodes the heavy chain. For example in one embodiment the first expression cassette comprises a polynucleotide encoding a heavy chain of an antigen binding protein comprising a constant region or antigen binding fragment thereof which is linked to a constant region according to the invention described herein and further comprises a second cassette comprising a polynucleotide encoding a light chain of an antigen binding protein comprising a constant region or antigen binding fragment thereof which is linked to a constant region according to the invention described herein for example the first expression cassette comprises a polynucleotide encoding a heavy chain selected from SEQ. ID. NO:18, or SEQ. ID. NO: 25 and a second expression cassette comprising a polynucleotide encoding a light chain selected from SEQ. ID. NO: 19 or SEQ. ID. NO: 27.

In another embodiment of the invention there is provided a stably transformed host cell comprising a vector comprising one or more expression cassettes encoding a heavy chain and/or a light chain of the antibody comprising a constant region or antigen binding fragment thereof which is linked to a constant region as described herein. For example such host cells may comprise a first vector encoding the light chain and a second vector encoding the heavy chain, for example the first vector encodes a heavy chain selected from SEQ. ID. NO: 18, or SEQ. ID. NO: 25 and a second vector encoding a light chain for example the light chain of SEQ ID NO: 19 or SEQ. ID. NO: 27. In one such example the first vector encodes a heavy chain selected from SEQ. ID. NO: 18 and a second vector encoding a light chain for example the light chain of SEQ ID NO: 19.

In another embodiment of the present invention there is provided a host cell according to the invention described herein wherein the cell is eukaryotic, for example where the cell is mammalian. Examples of such cell lines include CHO or NS0.

In another embodiment of the present invention there is provided a method for the production of an antibody comprising a constant region or antigen binding fragment thereof which is linked to a constant region according to the invention described herein which method comprises the step of culturing a host cell in a culture media, for example serum-free culture media.

In another embodiment of the present invention there is provided a method according to the invention described herein wherein said antibody is further purified to at least 95% or greater (e.g. 98% or greater) with respect to said antibody containing serum-free culture media.

In yet another embodiment there is provided a pharmaceutical composition comprising an antigen binding protein and a pharmaceutically acceptable carrier.

In another embodiment of the present invention there is provided a kit-of-parts comprising the composition according to the invention described herein described together with instructions for use.

The mode of administration of the therapeutic agent of the invention may be any suitable route which delivers the agent to the host. The antigen binding proteins, and pharmaceutical compositions of the invention are particularly useful for parenteral administration, i.e., subcutaneously (s.c.), intrathecally, intraperitoneally, intramuscularly (i.m.) or intravenously (i.v.). In one such embodiment the antigen binding proteins of the present invention are administered intravenously or subcutaneously.

Therapeutic agents of the invention may be prepared as pharmaceutical compositions containing an effective amount of the antigen binding protein of the invention as an active ingredient in a pharmaceutically acceptable carrier. In one embodiment the prophylactic agent of the invention is an aqueous suspension or solution containing the antigen binding protein in a form ready for injection. In one embodiment the suspension or solution is buffered at physiological pH. In one embodiment the compositions for parenteral administration will comprise a solution of the antigen binding protein of the invention or a cocktail thereof dissolved in a pharmaceutically acceptable carrier. In one embodiment the carrier is an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as about 15 or 20% by weight and will be selected primarily based on fluid volumes, viscosities, etc., according to the particular mode of administration selected.

Thus, a pharmaceutical composition of the invention for intravenous infusion could be made up to contain about 250 ml of sterile Ringer's solution, and about 1 to about 30 or 5 mg to about 25 mg of an antigen binding protein of the invention per ml of Ringer's solution. Actual methods for preparing parenterally administrable compositions are well known or will be apparent to those skilled in the art and are described in more detail in, for example, Remington's Pharmaceutical Science, 15^{th} ed., Mack Publishing Company, Easton, PA, USA. For the preparation of intravenously administrable antigen binding protein formulations of the invention see Parkins D. and Lasmar U. “The formulation of Biopharmaceutical products”, Pharm. Sci. Tech. Today, 3 (2000) 129-137; Wang, W “Instability, stabilisation and formulation of liquid protein pharmaceuticals”, Int. J. Pharm 185 (1999) 129-188; Jorgensen, L. et al, “Recent trends in stabilising peptides and proteins in pharmaceutical formulation-considerations in the choice of excipients” Expert Opin Drug Deliv. 6 (2009) 1219-1230; Akers, M. J. “Excipient-Drug interactions in Parenteral Formulations”, J. Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J. Pharm. Pharmacol, 54 (2002) 1033-1039; Johnson, R, et al “Mannitol-sucrose mixtures—versatile formulations for protein lyophilization”, J. Pharm. Sci, 91 (2002) 914-922; Kerwin B. “Polysorbates 20 and 80 used in the formulation of protein biotherapeutics: structure and degradation pathways” J. Pharm Sci. 97 (2008) 2924-2935; Ha, E., et al “Peroxide formation in polysorbate 80 and protein stability”, J. Pharm Sci, 91 (2002), 2252-2264, and He, F., et al, “Effect of sugar molecules on the viscosity of high concentration monoclonal antibody solutions” Pharm Res. 28 (2011) 1552-1560; and the entire contents of which are incorporated herein by reference and to which the reader is specifically referred.

In one embodiment the antibody of the invention, when in a pharmaceutical preparation, is present in unit dose forms. The appropriate therapeutically effective dose will be determined readily by those of skill in the art. Suitable doses may be calculated for patients according to their weight, for example suitable doses may be in the range of about 0.1 to about 200 mg/kg, for example about 1 to about 20 mg/kg, for example about 10 to about 20 mg/kg or for example about 1 to about 15 mg/kg, for example about 5 to about 15 mg/kg. To effectively treat conditions such as Multiple myeloma, SLE or IPT in a human, suitable doses may be within the range of about 0.1 to about 2000 mg, for example about 0.1 to about 500 mg, for example about 500 mg, for example about 0.1 to about 150 mg, or about 0.1 to about 80 mg, or about 0.1 to about 60 mg, or about 0.1 to about 40 mg, or for example about 1 to about 100 mg, or about 1 to about 50 mg, of an antigen binding protein of this invention, which may be administered parenterally, for example subcutaneously, intravenously or intramuscularly. Such dose may, if necessary, be repeated at appropriate time intervals selected as appropriate by a physician.

The antigen binding proteins described herein can be lyophilized for storage and reconstituted in a suitable carrier prior to use. This technique has been shown to be effective with conventional immunoglobulins and art-known peroxidise and reconstitution techniques can be employed.

In another aspect of the invention there is provided an antigen binding protein as herein described for use in a medicament.

In one aspect of the present invention there is provided an antigen binding protein according to the invention as herein described for use in the treatment of rheumatoid arthritis, Type 1 Diabetes Mellitus, multiple sclerosis or psoriasis wherein said method comprises the step of administering to said patient a therapeutically effective amount of the antigen binding protein as described herein.

In one embodiment of the present invention, methods are provided for treating cancer in a human comprising administering to said human an antigen binding protein that specifically binds to BCMA. In some instances the antigen binding protein is part of an immunoconjugate.

In another aspect of the present invention there is provided an antibody according to the invention as herein described for use in the treatment of a B-cell mediated or plasma cell mediated disease or antibody mediated disease or disorder selected from Multiple Myeloma (MM), chronic lymphocytic leukemia (CLL), Non-secretory multiple myeloma, Smoldering multiple myeloma, Monoclonal gammopathy of undetermined significance (MGUS), Solitary plasmacytoma (Bone, Extramedullary), Lymphoplasmacytic lymphoma (LPL), Waldenstrom's Macroglobulinemia, Plasma cell leukemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpasture's syndrome, Idiopathic thrombocytopenic purpura (ITP), Acute glomerulonephritis, Pemphigus and Pemphigoid disorders, and Epidermolysis bullosa acquisita; or any Non-Hodgkin's Lymphoma B-cell leukemia or Hodgkin's lymphoma (HL) with BCMA expression or any diseases in which patients develop neutralising antibodies to recombinant protein replacement therapy wherein said method comprises the step of administering to said patient a therapeutically effective amount of the antigen binding protein as described herein.

B-cell disorders can be divided into defects of B-cell development/immunoglobulin production (immunodeficiencies) and excessive/uncontrolled proliferation (lymphomas, leukemias). As used herein, B-cell disorder refers to both types of diseases, and methods are provided for treating B-cell disorders with an antigen binding protein.

In a particular aspect, the disease or disorder is selected from the group consisting of Multiple Myeloma (MM), Chronic Lymphocytic Leukaemia (CLL), Solitary Plasmacytoma (Bone, Extramedullary), Waldenstrom's Macroglobulinemia.

In one aspect of the present invention the disease is Multiple Myeloma, Smoldering Multiple Myeloma (SMM) or Solitary Plasmacytoma (Bone, Extramedullary).

In one aspect of the present invention the disease is Multiple Myeloma.

In one aspect of the present invention the disease is Systemic lupus erythematosus (SLE)

In one aspect of the present invention the disease is Idiopathic thrombocytopenic purpura (ITP)

Use of the antigen binding protein as described herein in the manufacture of a medicament for the treatment of diseases and disorders as described herein is also provided.

For example in one aspect of the invention there is provided the use of the antigen binding protein as described herein for use in the treatment or prophylaxis of diseases and disorders responsive to modulation (such as inhibiting or blocking) of the interaction between BCMA and the ligands BAFF and APRIL.

In another aspect of the invention there is provided the use of the antigen binding protein as described herein for use in the treatment or prophylaxis of an antibody mediated or plasma cell mediated disease or disorder selected from rheumatoid arthritis, Type 1 Diabetes Mellitus, multiple sclerosis or psoriasis.

In another aspect of the invention there is provided the use of the antibody as described herein for use in the treatment or prophylaxis of an antibody mediated or plasma cell mediated disease or disorder selected from Multiple Myeloma (MM), chronic lymphocytic leukemia (CLL), Monoclonal gammopathy of undetermined significance (MGUS), Smoldering multiple myeloma (SMM), Solitary Plasmacytoma (Bone, Extramedullary), Waldenstrom's Macroglobulinemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpastures syndrome, Idiopathic thrombocytopenic purpura (ITP), Acute glomerulonephritis, Pemphigus and Pemphigoid disorders and Epidermolysis bullosa acquisita, any Non-Hodgkin Lymphoma and Leukemia with BCMA expression or any diseases in which patients develop neutralising antibodies to recombinant protein replacement therapy wherein said method comprises the step of administering to said patient a therapeutically effective amount of the antigen binding protein as described herein.

In one aspect, the invention provides a pharmaceutical composition comprising an antibody of the present invention or a functional fragment thereof and a pharmaceutically acceptable carrier for treatment or prophylaxis of rheumatoid arthritis, Type 1 Diabetes Mellitus, multiple sclerosis or psoriasis or an antibody mediated or plasma cell mediated disease or disorder selected from selected from Multiple Myeloma (MM), chronic lymphocytic leukemia (CLL), Monoclonal gammopathy of undetermined significance (MGUS), Smoldering multiple myeloma (SMM), Solitary Plasmacytoma (Bone, Extramedullary), Waldenstrom's Macroglobulinemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpastures syndrome, Idiopathic thrombocytopenic purpura (ITP), Acute glomerulonephritis, Pemphigus and Pemphigoid disorders and Epidermolysis bullosa acquisita, any Non-Hodgkin Lymphoma and Leukemia with BCMA expression or any diseases in which patients develop neutralising antibodies to recombinant protein replacement therapy wherein said method comprises the step of administering to said patient a therapeutically effective amount of the antigen binding protein as described herein.

In another embodiment of the present invention there is provided a method of treating a human patient afflicted with rheumatoid arthritis, Type 1 Diabetes Mellitus, multiple sclerosis or psoriasis or an antibody mediated or plasma cell mediated disorder or disease which method comprises the step of administering a therapeutically effective amount of the antigen binding protein according to the invention as described herein, for example there is provided a method of treating a human patient afflicted with an antibody mediated or plasma cell mediated disease or disorder selected from In another aspect of the present invention there is provided an antigen binding protein according to the invention as herein described for use in the treatment of an antibody mediated or plasma cell mediated disease or disorder selected from Multiple Myeloma (MM), Chronic Lymphocytic Leukaemia (CLL) Monoclonal gammopathy of undetermined significance (MGUS), Smoldering multiple myeloma (SMM), Solitary Plasmacytoma (Bone, Extramedullary), Waldenstrom's Macroglobulinemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpastures syndrome, Idiopathic thrombocytopenic purpura (ITP), Acute glomerulonephritis, Pemphigus and Pemphigoid disorders and Epidermolysis bullosa acquisita, any Non-Hodgkin Lymphoma and Leukemia with BCMA expression or any diseases in which patients develop neutralising antibodies to recombinant protein replacement therapy wherein said method comprises the step of administering a pharmaceutical composition comprising an antigen binding protein according to the invention herein in combination with a pharmaceutically acceptable carrier.

In a further embodiment there is provided a method of treating a human patient afflicted with Multiple Myeloma (MM).

The BCMA antibody described herein is useful for any therapeutic in which it is desirable to target BCMA, such as adoptive cell transfer (ACT), bispecific T-cell engagers (BiTEs), and nanoparticles. In one embodiment, the disclosure provides a chimeric antigen receptor (CAR) comprising an antigen binding domain of the BCMA monoclonal antibody described herein linked to a T-cell activation moiety. A “chimeric antigen receptor (CAR)” is an artificially constructed hybrid protein or polypeptide containing an antigen binding domain of an antibody (e.g., a single chain variable fragment (scFv)) linked to T-cell signaling or T-cell activation moeities. CAR structures have evolved over the last twenty years to most commonly incorporate a single chain variable fragment (scFv) derived from a monoclonal antibody (mAb) and the signaling motif from the TCR chain (referred to as a “first-generation” CAR (see, e.g., Okur, F. V., Brenner, M. K., Methods Mol. Biol., 651: 319-45 (2010); and Lee et al., Clin. Cancer. Res., 18(10): 2780-2790 (2012)). More recently, second and third generation CARs have been developed, which incorporate one (“second generation”) or two (“third generation”) costimulatory activating motifs from, for example, CD28, 4-1BB (CD137), and/or CD134 (OX-40), which enhance proliferation, cytotoxicity, and persistence in vivo (see, e.g., Finney et al., J. Immunol., 172: 104-13 (2004); Altvater et al, Clin Exp Immunol. 144(3): 447-57 (2006); Chu et al, J Transl Med. 20(1): 240 (2022); Maher et al., Nat Biotechnol., 20:70-5 (2002); Milone et al., Mol Ther., 17: 1453-64 (2009); SafarzadehKozani et al, Biomark Res. 10(1): 24 (2022); Xu et al, Blood Sci. 1(2):156-160 (2019) and Qian et al., Front Immunol. 13: 841425 (2022)).

The antigen binding domain of the CAR may comprise a whole monoclonal antibody or a monoclonal antibody fragment, as described herein. In one embodiment, the antigen binding domain of the CAR may comprise a single chain Fv (scFv) fragment of the anti-BCMA monoclonal antibody. Chimeric antigen receptors and methods for generating CARs are further described in, for example, Ohmine K, and Uchibori R. Int J Hematol. 115(6): 799-810 (2022); Ding L, et al, Stem Cell Investig. 8: 1 (2021); Riviere, I. and M. Sadelain, Mol. Ther., 25(5): 1117-1124 (2017); Chan L. Y. et al, Biomedicines. 10(4): 804 (2022); Davila, M. L. and M. Sadelain, Int. J. Hematol., 104(1): 6-17 (2016); and Mohty et al, Leukemia. 33(12): 2767-2778 (2019).

The term “antibody-drug conjugate (ADC),” as used herein, refers to a compound comprising a monoclonal antibody (mAb) attached to a cytotoxic agent (generally a small molecule drug with a high systemic toxicity) via chemical linkers. The ADC is represented as the formula of

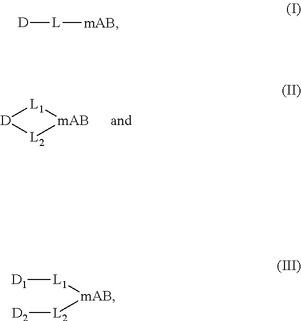

wherein D, D₁ and D₂ area small molecule cytotoxin or a functional small molecule, in general called payload; L, L₁ and L₂ are a linker; and mAb is an monoclonal antibody. In some embodiments, an ADC may comprise a small molecule cytotoxin that has been chemically modified to contain a linker, or a linker is part of payload which is called a traceless linker. The linker is generally used to conjugate the cytotoxin to the antibody, or antigen-binding fragment thereof. Upon binding to the target antigen on the surface of a cell, the ADC is internalized and trafficked to the lysosome where the cytotoxin is released by either proteolysis of a cleavable linker (e.g., by cathepsin B found in the lysosome) or by proteolytic degradation of the antibody, if attached to the cytotoxin via a non-cleavable linker. The cytotoxin then translocates out of the lysosome and into the cytosol or nucleus, where it can then bind to its target, depending on its mechanism of action.

The antibody-drug conjugate described herein may comprise a whole antibody or an antibody fragment. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have the same general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

The ADC may comprise an antigen-binding fragment of an antibody. The terms “antibody fragment,” “antigen-binding fragment,” “functional fragment of an antibody,” and “antigen-binding portion” are used interchangeably herein and refer to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Kabat E A, Wu T T., J Immunol. 1991, 147(5): 1709-19) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites (see, e.g. Hudson P J, Kortt A A, J Immunol Methods. 1999, 231(1-2): 177-89; Holliger P, Winter G. Cancer Immunol Immunother. 1997, 45(3-4):128-30).

In one embodiment, the antibody-drug conjugate described herein comprises a monoclonal antibody, or an antigen-binding fragment thereof, directed against B-cell Maturation Antigen (BCMA, also known as CD269). The monoclonal antibody, or antigen-binding fragment thereof, may comprise (a) a heavy chain variable region comprising a complementarity determining region 1 (HCDR1) amino acid sequence of SEQ ID NO: 1, an HCDR2 amino acid sequence of SEQ ID NO: 2, and an HCDR3 amino acid sequence of SEQ ID NO: 3 and (b) a light chain variable region comprising a complementarity determining region 1 (LCDR1) amino acid sequence of SEQ ID NO: 4, an LCDR2 amino acid sequence of SEQ ID NO: 5, and an LCDR3 amino acid sequence of SEQ ID NO: 6. In another embodiment, the monoclonal antibody comprises a heavy chain variable region comprising the amino acid sequence of SEQ ID NO: 8 and/or a light chain variable region comprising the amino acid sequence of SEQ ID NO: 9.

The monoclonal antibody, or an antigen-binding fragment thereof, directed against BCMA may comprise any suitable binding affinity to BCMA or an epitope thereof. The term “affinity” refers to the equilibrium constant for the reversible binding of two agents and is expressed as the dissociation constant (K_{D}). The affinity of an antibody or antigen-binding fragment thereof for an antigen or epitope of interest can be measured using any method known in the art. Such methods include, for example, fluorescence activated cell sorting (FACS), surface plasmon resonance (e.g., Biacore™ ProteOn™), biolayer interferometry (BLI, e.g. Octet), kinetics exclusion assay (e.g. KinExA™) separable beads (e.g., magnetic beads), antigen panning, and/or ELISA (see, e.g., J R Crowther, Methods Mol Biol. 2000,149:III-IV, 1-413). It is known in the art that the binding affinity of a particular antibody will vary depending on the method that is used to analyze the binding affinity.

Affinity of a binding agent to a ligand, such as affinity of an antibody for an epitope, can be, for example, from about 1 picomolar (pM) to about 1 micromolar (1 pM) (e.g., from about 1 picomolar (pM) to about 1 nanomolar (nM), or from about 1 nM to about 1 micromolar (pM)). In one embodiment, the monoclonal antibody or an antigen-binding fragment thereof may bind to BCMA with a Kd less than or equal to 100 nanomolar (e.g., 100 nM, about 90 nM, about 80 nM, about 70 nM, about 60 nM, about 50 nM, about 40 nM, about 30 nM, about 20 nM, or about 10 nM, or a range defined by any two of the foregoing values).

In another embodiment, the monoclonal antibody may bind to BCMA with a Kd less than or equal to 10 nanomolar (e.g., about 9 nM, about 8 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.05 nM, about 0.02 nM, about 0.01 nM, about 0.001 nM, or a range defined by any two of the foregoing values).

In another embodiment, the monoclonal antibody may bind to BCMA with a Kd less than or equal to 200 pM (e.g., about 190 pM, about 175 pM, about 150 pM, about 125 pM, about 110 pM, about 100 pM, about 90 pM, about 80 pM, about 70 pM, about 60 pM, about 50 pM, about 40 pM, about 30 pM, about 25 pM, about 20 pM, about 15 pM, about 10 pM, about 5 pM, about 1 pM, or a range defined by any two of the foregoing values).

In one embodiment, the affinity of the BCMA antibody or antigen-binding fragment thereof to monomeric BCMA, as measured by surface plasmon resonance (SPR), is about 90 nM, about 80 nM, about 70 nM, about 60 nM, about 50 nM, about 40 nM, about 30 nM, or a range defined by any two of the foregoing values, for example, about 50 nM to about 70 nM, about 55 nM to about 65 nM, or about 58 nM to about 62 nM.

In one embodiment, the affinity of the BCMA antibody or antigen-binding fragment thereof to membrane-bound BCMA, as measured by FACS, is less than or equal to 10 nanomolar (e.g., about 9 nM, about 8 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.05 nM, about 0.02 nM, about 0.01 nM, about 0.001 nM, or a range defined by any two of the foregoing values).

An antigen-binding portion or fragment of a monoclonal antibody can be of any size so long as the portion binds to BCMA. In this respect, an antigen binding portion or fragment of the monoclonal antibody directed against BCMA (also referred to herein as an “anti-BCMA monoclonal antibody”) desirably comprises between about 5 and 25 amino acids (e.g., about 5, 6, 7, 8, 9, 10, 11, 12, 13, 14, 15, 16, 17, 18, 19, 20, 21, 22, 23, 24, 35 or a range defined by any two of the foregoing values).

In one embodiment, the antibody-drug conjugate comprises a variable region of an anti-BCMA monoclonal antibody. In this respect, the ADC may comprise a light chain variable region, a heavy chain variable region, or both a light chain variable region and a heavy chain variable region of an anti-BCMA monoclonal antibody. Preferably, the ADC comprises a light chain variable region and a heavy chain variable region of an anti-BCMA monoclonal antibody. In one embodiment, the monoclonal antibody of the ADC described herein comprises (a) a heavy chain variable region comprising a complementarity determining region 1 (HCDR1) amino acid sequence of TSFLIHW (SEQ ID NO: 1), an HCDR2 amino acid sequence of FIIPGNGGTKYNQKFQ (SEQ ID NO: 2), and an HCDR3 amino acid sequence of YDGSFEGYFDV (SEQ ID NO: 3) and (b) a light chain variable region comprising a complementarity determining region 1 (LCDR1) amino acid sequence of SSQSLVHSDGNTYLH (SEQ ID NO: 4), an LCDR2 amino acid sequence of KVSNRDS (SEQ ID NO: 5), and an LCDR3 amino acid sequence of SQSTHWPWT (SEQ ID NO: 6). In another embodiment, the monoclonal antibody of the ADC described herein may comprise a heavy chain variable region comprising the amino acid sequence of SEQ ID NO: 8 and/or a light chain variable region comprising the amino acid sequence of SEQ ID NO: 9.

The BCMA monoclonal antibody, or antigen-binding fragment thereof, may be conjugated to a cytotoxin using any suitable method known in the art, including site-specific or non-site specific conjugation methods. Conventional conjugation strategies for antibodies typically rely on randomly (i.e., non-specifically) conjugating the payload to the antibody, antigen-binding fragment thereof, through lysines or cysteines. Accordingly, in some aspects the antibody or antigen-binding fragment thereof is randomly conjugated to a cytotoxic agent, for example, by partial reduction of the antibody or antibody fragment, followed by reaction with a desired agent with or without a linker moiety attached. For example, the antibody or antigen-binding fragment thereof may be reduced using dithiothreitol (DTT), TCEP, thiolethenol or a similar reducing agent. The cytotoxic agent, with or without a linker moiety attached thereto, can then be added at a molar excess to the reduced antibody or antibody fragment in the presence of dimethyl sulfoxide (DMSO), or DMA. After conjugation, excess free cysteine may be added to quench unreacted agent. The cytotoxic agent, with or without a linker moiety having an amino-reactivable, or phenol-reactivable, or the others reactivable group (e.g. NHS, PFP) thereto, can be added directly at a molar excess to the antibody or antibody fragment in the presence of DMSO, or DMA to form a conjugate. The reaction mixture may then be purified through chromatography or buffer-exchanged into phosphate buffered saline (PBS).

The terms “cytotoxin” and “cytotoxic agent” refer to any molecule that inhibits or prevents the function of cells and/or causes destruction of cells (cell death), and/or exerts anti-proliferative effects. A cytotoxin or cytotoxic agent of an ADC also is referred to in the art as the “payload” of the ADC. A number of classes of cytotoxic agents are known in the art to have potential utility in ADC molecules and can be used in the ADC described herein. Such classes of cytotoxic agents include, for example, anti-microtubule agents (e.g., tubulysins, auristatins and maytansinoids), DNA minor groove binders (e.g. pyrrolobenzodiazepines (PBDs) or indolinobenzodiazepines (IGN)), RNA polymerase II inhibitors (e.g., amatoxins), inhibitor of DNA topoisomerase I (e.g., camptothecins) and DNA alkylating agents (e.g., duocarmycin, CC-1065, pyrrolobenzodiazepine or indolinobenzodiazepinepseudodimers). Examples of specific cytotoxic agents that may be used in the ADC described herein include, but are not limited to, tubulysins, amanitins, auristatins, calicheamicin, camptothecins, daunomycins, doxorubicins, duocarmycins, dolastatins, enediynes, lexitropsins, taxanes, puromycins, maytansinoids, vinca alkaloids, and pyrrolobenzodiazepines (PBDs). More specifically, the cytotoxic agent may be, for example tubulysins, auristatins (AFP, MMAF, MMAE, AEB, AEVB, E), paclitaxels, docetaxels, CC-1065 (ducarmysin, DC1, DC4, CBI-dimers), camptothecins (SN-38, topotecans), morpholino-doxorubicin, rhizoxin, cyanomorpholino-doxorubicin, dolastatin-10, echinomycin, combretatstatin, chalicheamicin, maytansine (DM1, DM4, DM21), vinblastine, methotrexate, netropsin, or derivatives or analogs thereof. Cytotoxins suitable for use in ADCs are also described in, for example, International Patent Application Publication No. PCT/CN2021/128453.

In general, a chemotherapeutic agent or a functional compound can also be conjugated to the BCMA antibody of this invention. A chemotherapeutic agent or a functional compound is selected from the group consisting of:
a). an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine(PBD)dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). A plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; erbulins, a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid}; and Inhibitors of nicotinamide phosphoribosyltransferase (NAMPT);
e). A hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (comprising CB 1093, EB 1089 KH 1060, cholecalciferol, ergocalciferol); Photodynamic therapies: (comprising verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group consisting of olaparib, niraparib, iniparib, talazoparib, veliparib, CEP 9722 (Cephalon's), E7016 (Eisai's), BGB-290 (BeiGene's), or 3-aminobenzamide.
h). An antibiotic, selected from the group consisting of an enediyne antibiotic (selected from the group consisting of calicheamicin, calicheamicin γ1, δ1, α1 or β1; dynemicin, including dynemicin A and deoxydynemicin; esperamicin, kedarcidin, C-1027, maduropeptin, orneocarzinostatin chromophore and related chromoprotein enediyne antibioticchromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins and carfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((comprising Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; razoxane; rhizoxin; sizofiran; spirogermanium; tenuazonic acid; triaziquone; 2, 2′,2″-trichlorotriethylamine; trichothecenes (including the group consisting of T-2 toxin, verrucarin A, roridin A and anguidine); urethane, siRNA, antisense drugs;
(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.
(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem/cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). Glycopeptides: bleomycin, vancomycin (oritavancin, telavancin), teicoplanin (dalbavancin), ramoplanin;
g). Glycylcyclines: tigecycline;
h). β-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). Other antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU),3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.
(5). A radioisotope that can be selected from the group consisting of (radionuclides) ³H, ¹¹C, ¹⁴C, ¹⁸F, ³²p, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi.
(6). A chromophore molecule, which is capable of absorbing UV light, florescent light, IR light, near IR light, visual light; A class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; Non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170). Acridine derivatives (comprising proflavin, acridine orange, acridine yellow). Arylmethine derivatives (comprising auramine, crystal violet, malachite green). Tetrapyrrole derivatives (comprising porphin, phthalocyanine, bilirubin); Any analogs and derivatives of the following fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; A fluorophore compound: comprising DCFH (2′7′Dichorodihydro-fluorescein, oxidized form), DHR (Dihydrorhodamine 123, oxidized form, light catalyzes oxidation), Fluo-3 (AM ester. pH>6), Fluo-4 (AM ester. pH 7.2), Indo-1 (AM ester, low/high calcium (Ca2+)), SNARF (pH 6/9), Allophycocyanin (APC), AmCyan1 (tetramer, Clontech), AsRed2 (tetramer, Clontech), Azami Green (monomer), Azurite, B-phycoerythrin (BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreen1 (tetramer), ZsYellow1 (tetramer) and their derivatives.
(7). The cell-binding ligands or receptor agonists, which can be selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gy-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gy-NH₂), Nafarelin Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and NodlikeReceptors' (NLRs) ligands; Calcitonin receptor agonists; integrin receptors' and their receptor subtypes' (selected from the group consisting of αvβ₁, αvβ₃, αvβ₅, αvβ₆, α₆β₄, α₇β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists.
(8). The pharmaceutically acceptable salts, acids, derivatives, hydrate or hydrated salt; or a crystalline structure; or an optical isomer, racemate, diastereomer or enantiomer of any of the above drugs.

In another embodiment, the drug D can be polyalkylene glycols that are used for extending the half-life of the cell-binding antibody, or antibody molecule when administered to a mammal. Polyalkylene glycols include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodoacetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones).

In certain such embodiments, the polyalkylene glycol has a molecular weight of from about 10 Daltons to about 200 kDa, preferably about 88 Da to about 40 kDa; two branches each with a molecular weight of about 88 Da to about 40 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728, 560; 5,730,990; 5,738,846; 5,811,076; 5,824,701; 5,840,900; 5,880,131; 5,900,402; 5,902,588; 5,919,455; 5,951,974; 5,965,119; 5,965,566; 5,969,040; 5,981,709; 6,011,042; 6,042,822; 6,113,906; 6,127,355; 6,132,713; 6,177,087, and 6,180,095.

In yet another embodiment, D is more preferably a potent cytotoxic agent, selected from a tubulysin and its analogs, a maytansinoid and its analogs, a taxanoid (taxane) and its analogs, a CC-1065 and its analogs, a daunorubicin or doxorubicin and its analogs, an amatoxin and its analogs, a benzodiazepine dimer (e.g., dimers of pyrrolobenzodiazepine (PBD), tomaymycin, anthramycin, indolinobenzodiazepines, imidazobenzothiadiazepines, or oxazolidinobenzo-diazepines) and their analogs, a calicheamicin and the enediyne antibiotic and their analogs, an actinomycin and its analogs, an azaserine and its analogs, a bleomycin and its analogs, an epirubicin and its analogs, a tamoxifen and its analogs, an idarubicin and its analogs, a dolastatin and its analogs, an auristatin (including monomethyl auristatin E (MMAE), MMAF, auristatin PYE, auristatin TP, Auristatins 2-AQ, 6-AQ, EB (AEB), and EFP (AEFP)) and its analogs, a combretastatin, a duocarmycin and its analogs, a camptothecin, a geldanamycin and its analogs, a methotrexate and its analogs, a thiotepa and its analogs, a vindesine and its analogs, a vincristine and its analogs, a hemiasterlin and its analogs, a nazumamide and its analogs, a spliceostatin, a pladienolide, a microginin and its analogs, a radiosumin and its analogs, an alterobactin and its analogs, a microsclerodermin and its analogs, a theonellamide and its analogs, an esperamicin and its analogs, PNU-159682 and its analogs, a protein kinase inhibitor, a MEK inhibitor, a KSP inhibitor, a nicotinamide phosphoribosyltransferase (NAMPT)inhibitor, an immunotoxin, and stereoisomers, isosteres, analogs, or derivatives above thereof.

Tubulysin and its analogs are well known in the art and can be isolated from natural sources according to known methods or prepared synthetically according to known methods (e. g. Balasubramanian, R., et al. J. Med. Chem., 2009, 52, 238-40; Wipf, P., et al. Org. Lett., 2004, 6, 4057-60; Pando, O., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, O., et al. Org. Lett., 2009, 11 (24), 5567-9; Wipf, P., et al. Org. Lett., 2007, 9 (8), 1605-7; Friestad, G. K., Org. Lett., 2004, 6, 3249-52; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49: 4809-12; Chai, Y., et al., Chem Biol, 2010, 17: 296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; Patent applications: Zanda, M., et al, Can. Pat. Appl. CA 2710693 (2011); Chai, Y., et al. Eur. Pat. Appl. 2174947 (2010), WO 2010034724; Leamon, C. et al, WO2010033733, WO 2009002993; Ellman, J., et al, PCT WO2009134279; WO 2009012958, US appl. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. Pat. Appl. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., US appl. 20110027274. The preferred structures of tubulysins for conjugation of cell binding molecules through process of the present patent application are described in the patent application of PCT/IB2012/053554.

Tubulysin analog having the following formula (IV):

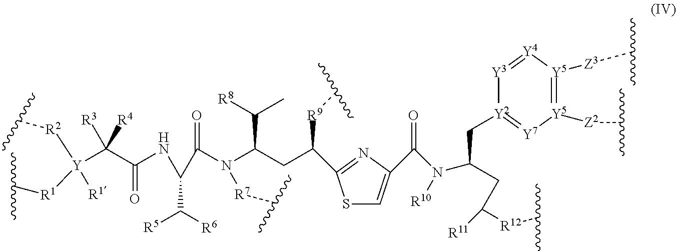

or a pharmaceutically acceptable salt, hydrates, or hydrated salt; or a polymorphic crystalline structure; or an optical isomer, racemate, diastereomer or enantiomer thereof,
wherein [symbol] is a linkage site that either one or two of them can link to L₁ and/or L₂ independently; when two of “ [symbol] ” link to both L₁ and L₂, R¹ and R², or Z² and Z³ are preferably the dual linkage sites;
wherein R¹, R^{1′}, R², R³, and R⁴ are independently H, C₁˜C₈ alkyl; C₂˜C₈heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or R¹R², R¹R³, R²R³, R³R⁴, or R⁵R⁶ form a 3˜7 membered carbocyclic, cycloalkyl, heterocyclic, heterocycloalkyl, aromatic or heteroaromatic ring system; R¹ and R² can be independently absent when they link to L₁ or L₂ independently or simultaneously, Y¹ is N or CH;
wherein R⁵, R⁶, R⁸, R¹⁰ and R¹¹ are independently H, or C₁˜C₄ alkyl or heteroalkyl;
wherein R⁷ is independently H, R¹⁴, —R¹⁴C(═O)X¹R¹⁵; or —R¹⁴X¹R¹⁵; X¹ is O, S, S—S, NH, CH₂ or NR¹⁴;
wherein R⁹ is selected from H, OH, ═O, —OR¹⁴, —OC(═O)R¹⁴, —OC(═O)NHR¹⁴, —OC(═O)NR¹⁴R¹⁵, OP(═O)(OR¹⁴)₂, —OC(═O)NR¹⁴R¹⁵, or OR¹⁴OP(═O)(OR¹⁵)₂; when R⁹ links L₁ or L₂, R⁹ is, —O—, —OC(═O)NH— or —OC(═O)N(R¹⁴)—;
wherein R¹¹ is independently H, R¹⁴, —R¹⁴C(═O)R¹⁵, —R¹⁴C(═O)X²R¹⁵, wherein X² is —O—, —S—, —NH—, or —N(R¹⁴)—;
wherein R¹² is —COOH, —COSH, —CONH₂, CONHNH₂, CONHNHR¹⁵, —CONH(R¹⁵), —COOR¹⁵, —R¹⁵COR¹⁶, —R¹⁵COOR¹⁶, —R¹⁵C(O)NH₂, —R¹⁵C(O)NHR¹⁶, —COSR¹⁵, R¹⁵S(═O)₂R¹⁶, —R¹⁵P(═O)(OR¹⁷)₂, —R¹⁵OP(═O)(OR¹⁷)₂, —COOCH₂OP(═O)(OR¹⁷)₂, —COX²SO₂R¹⁷, —COOR¹⁵X²R¹⁶, tetrazole, imidazole, or triazole, where X² is —O—, —S—, —NH—, —N(R¹⁵)—, —O—R¹⁵—, —S—R¹⁵—, CH₂ or —NHR¹⁵—; when R¹² links L₁ or L₂, R¹² is —C(O)O—, —C(O)NH—, —C(═O)NHS(O)₂R¹⁵— or —C(═O)N(R¹⁵)—;
R¹³ and R¹⁴ are independentlyC₁˜C₈ alkyl, heteroalkyl; C₂-C₈ of alkenyl, alkynyl, heteroalkyl, heterocycloalkyl; C₃-C₈ of aryl, Ar-alkyl;
Z² and Z³ are independently H, O, S, NH, N(R¹⁵), NHNH, —OH, —SH, —NH₂, NH, NHNH₂, —NH(R¹⁵), —OR¹⁵, CO, —COX², —COX²R¹⁶, R¹⁷, F, Cl, Br, I, SR¹⁶, NR¹⁶R¹⁷, N═NR¹⁶, N═R¹⁶, N₀₂, SOR¹⁶R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷; —O—(C₄-C₁₂ glycoside), —N—(C₄-C₁₂ glycoside); C₁˜C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)_{1˜8}, or CO(Aa)_{1˜8} (which are respectively N-terminal or C-terminal 1-8 the same or different amino acids)), or polyethyleneoxy unit of formula (OCH₂CH₂)ₚ or (OCH₂CH(CH₃))ₚ, wherein p is an integer from 0 to about 1000, or combination of above groups thereof, X² is O, S, S—S, NH, CH₂, OH, SH, NH₂, CHR¹⁵ or NR¹⁵;
R¹⁵, R¹⁶ and R¹⁷ are independently H, C₁˜C₈ alkyl, heteroalkyl; C₂-C₈ of alkenyl, alkynyl, heteroalkyl, heterocycloalkyl; C₃-C₈ of aryl, Ar-alkyl, cycloalkyl, heteroalkylcycloalkyl, alkylcarbonyl, heteroaryl, alkylcarbonyl, or Na⁺, K⁺, Cs⁺, Li⁺, Ca²⁺, Mg⁺, Zn²⁺, N⁺(R¹)(R²)(R³)(R⁴), HN⁺(C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N (R1), thus Y², Y⁴, Y⁵, Y⁶ and Y⁷ form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y⁴, Y⁵, Y⁶ and Y⁷ form aromatic ring of benzene, pyridine, pyridazine, pyrimidine, pyrazine, triazine, tetrazine, pentazine;

Examples of the structures of the tubulysin analogs are shown below:

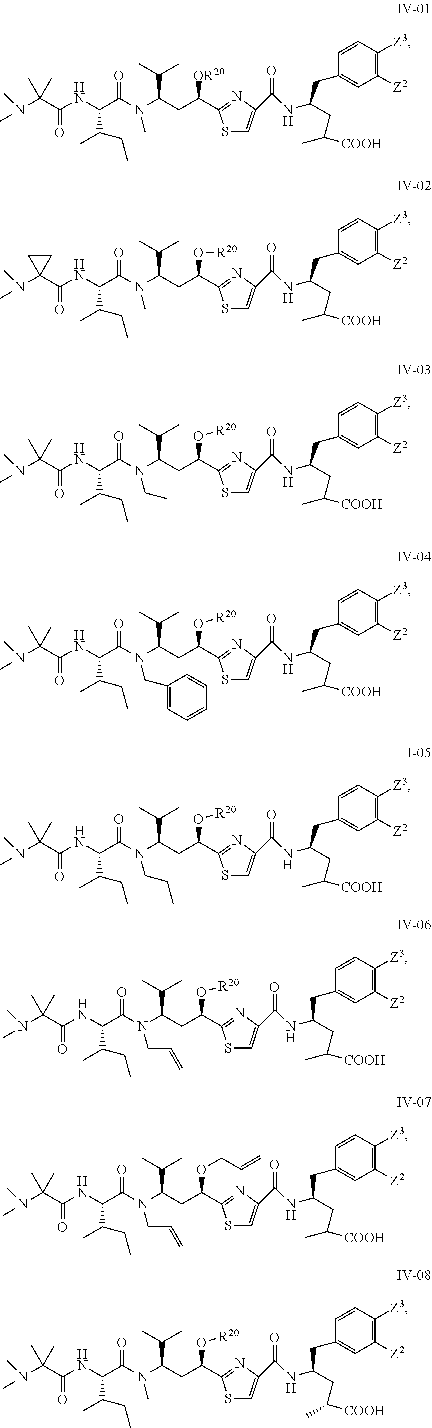

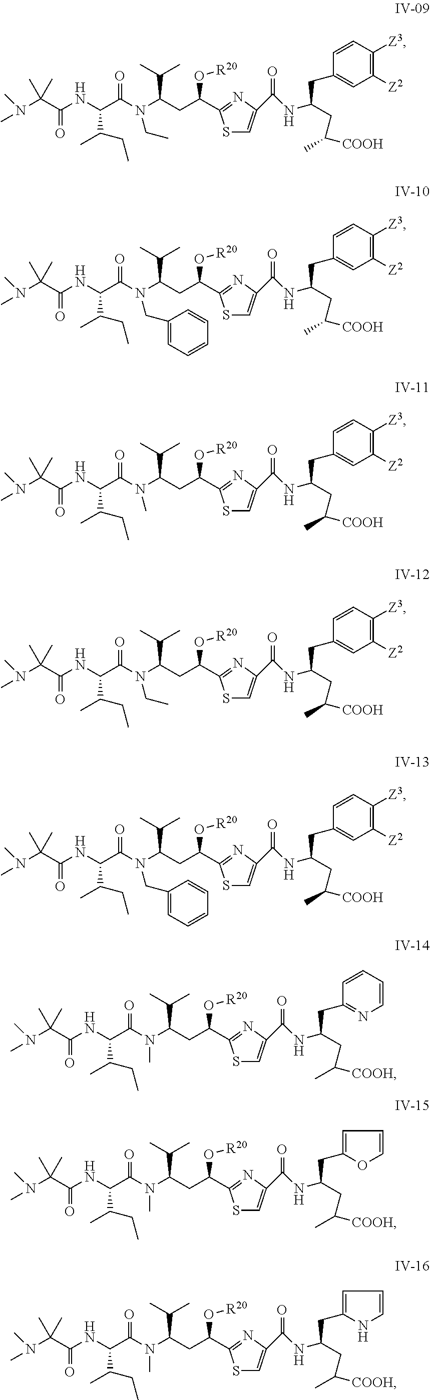

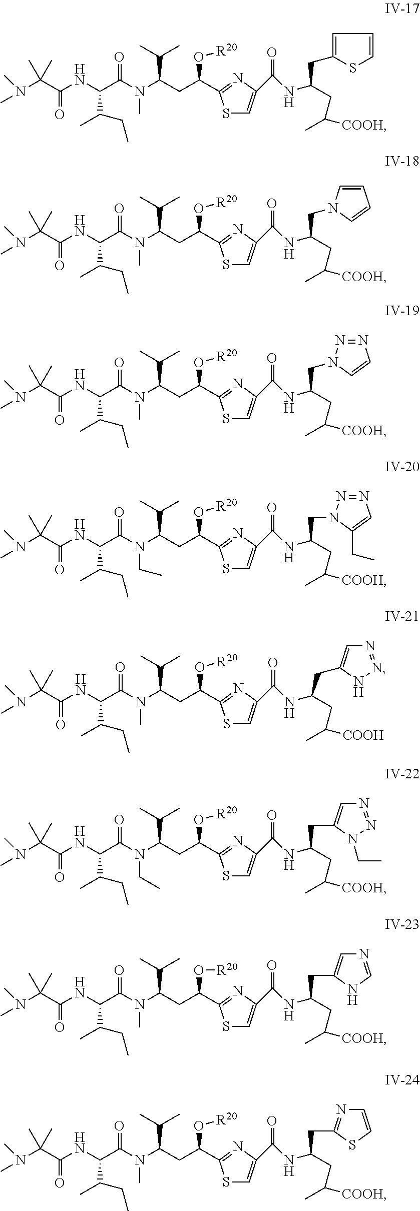

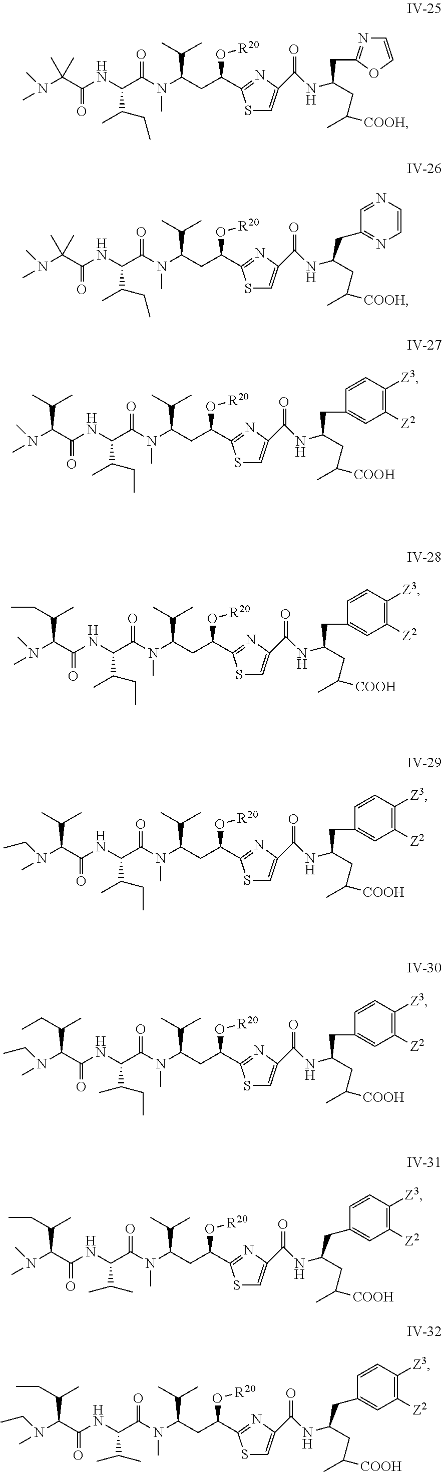

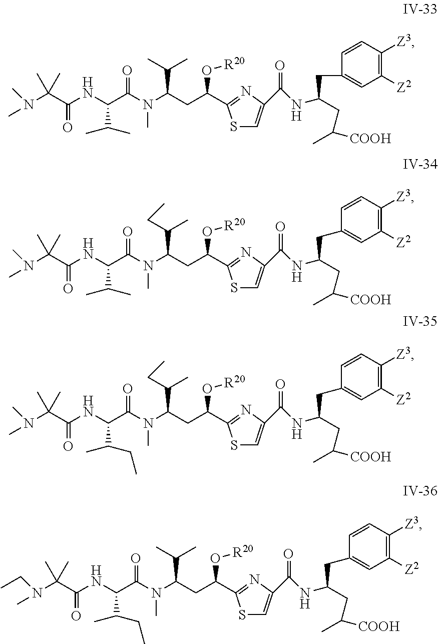

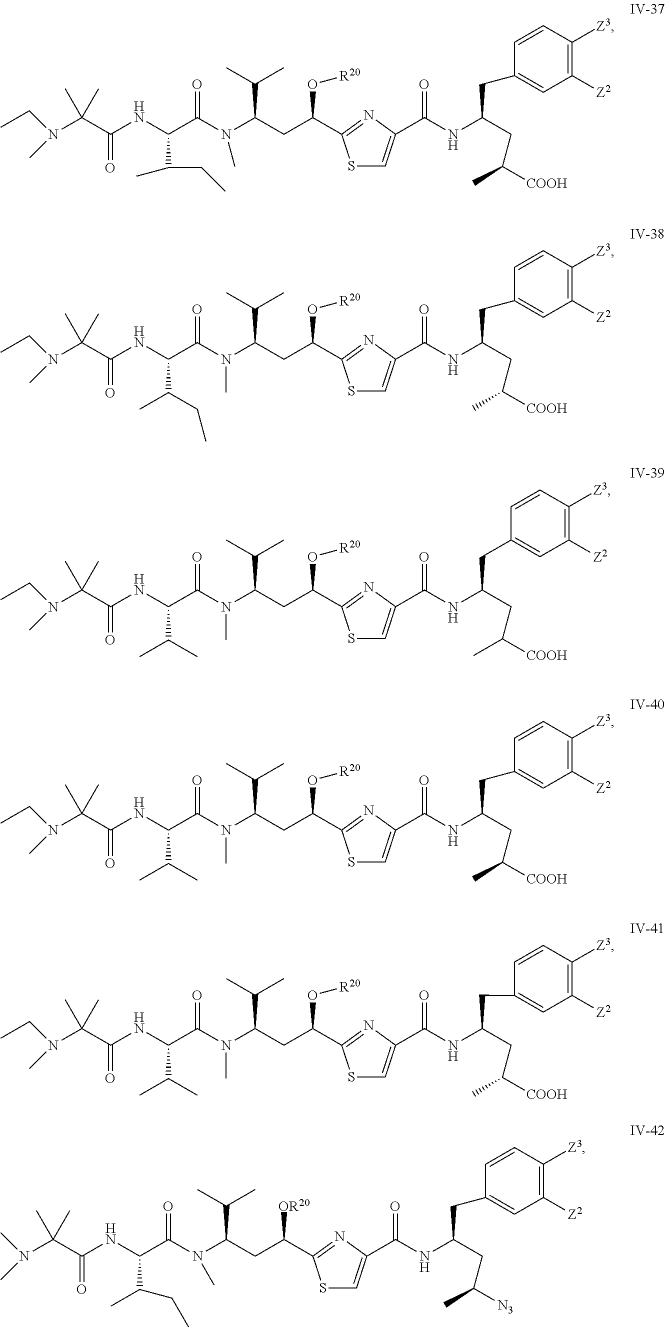

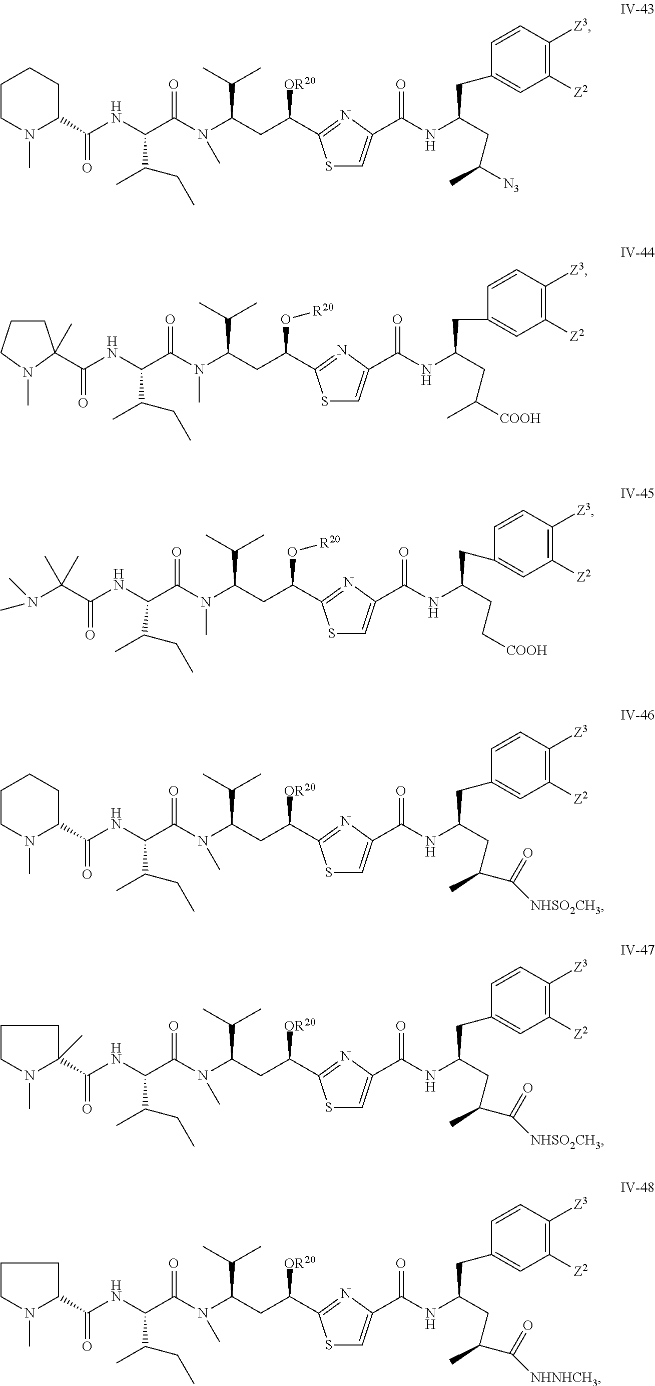

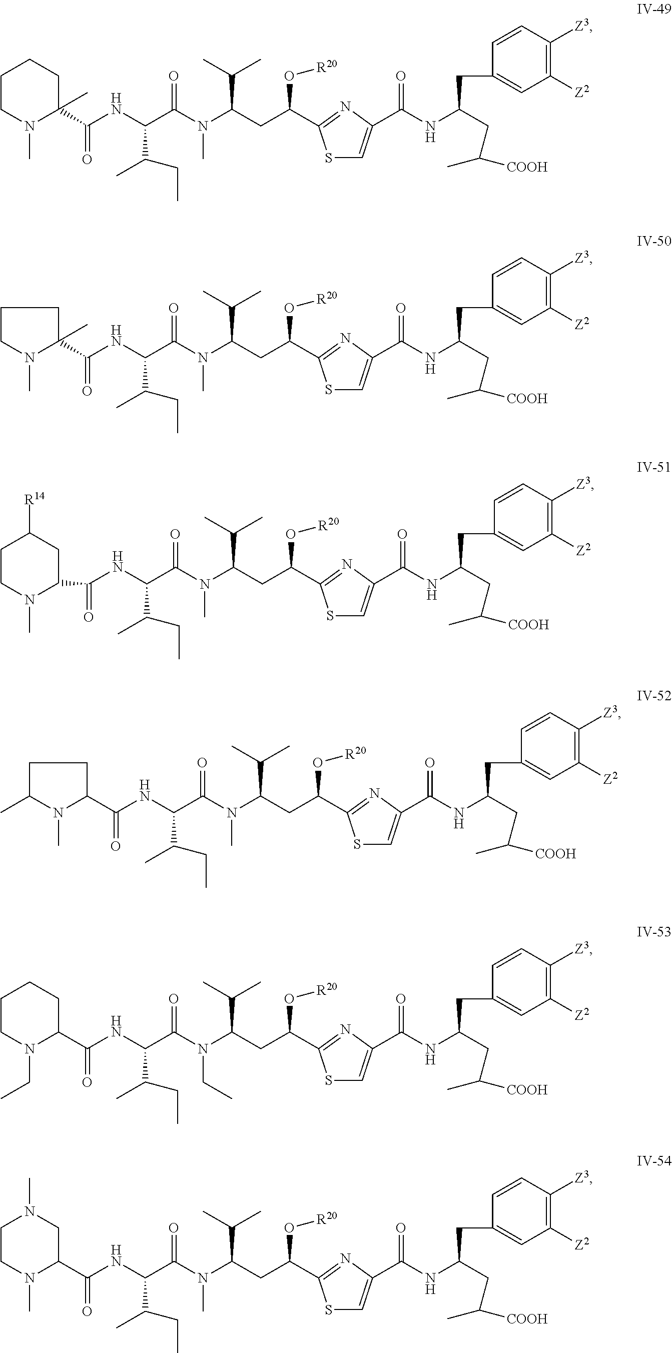

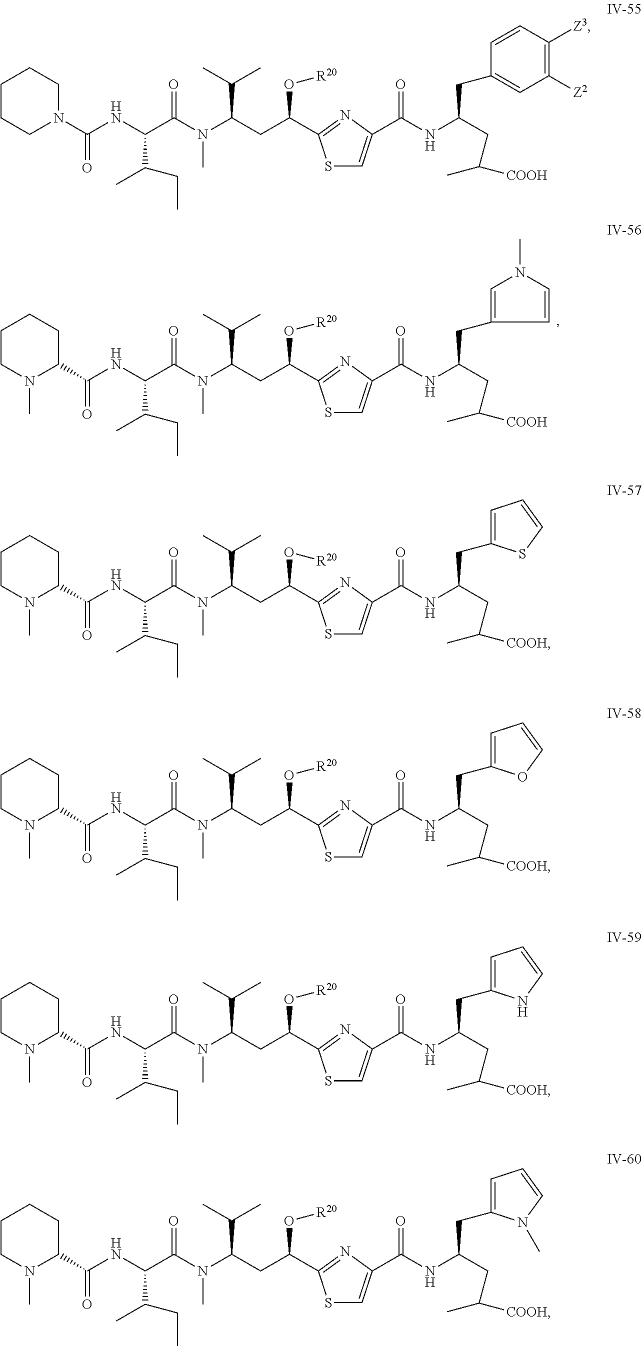

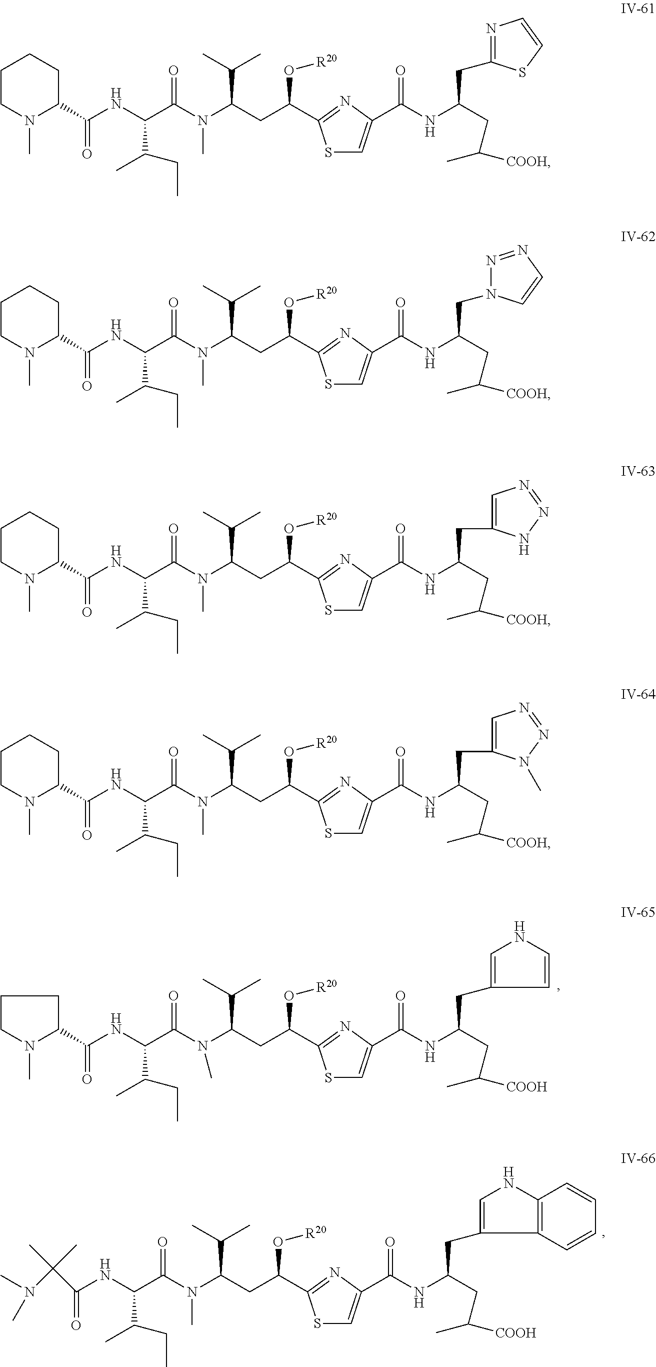

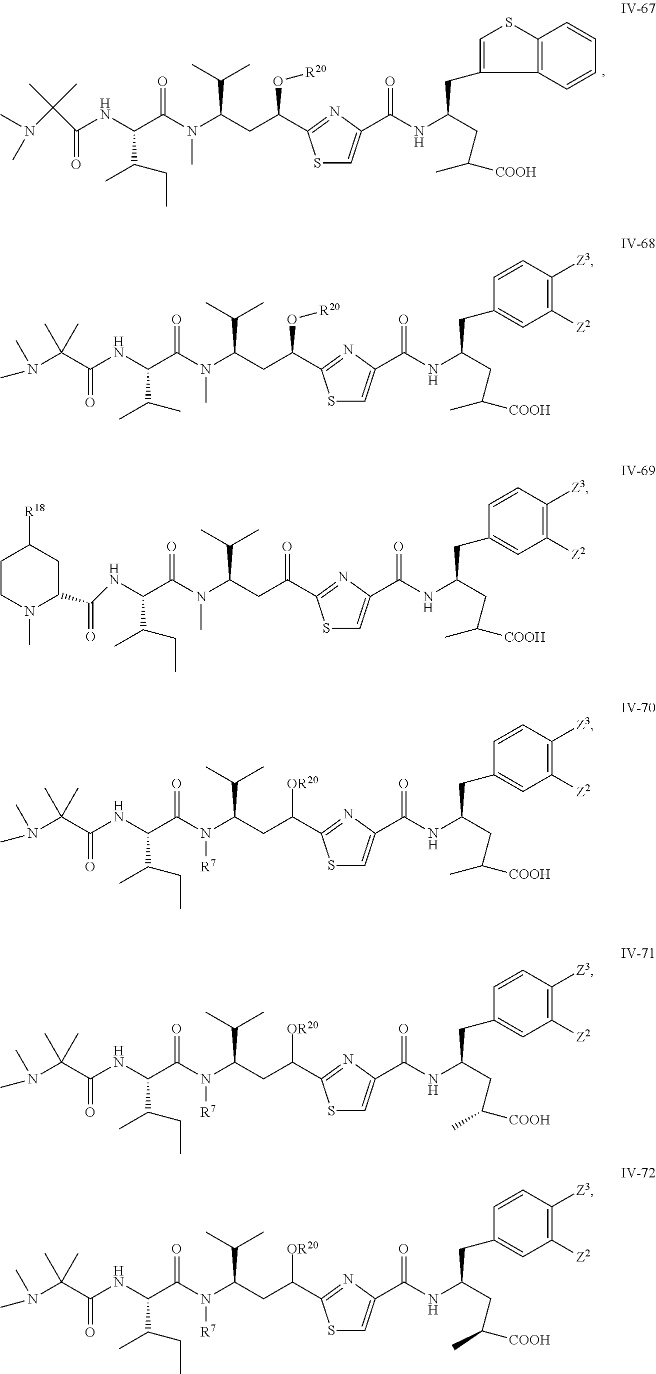

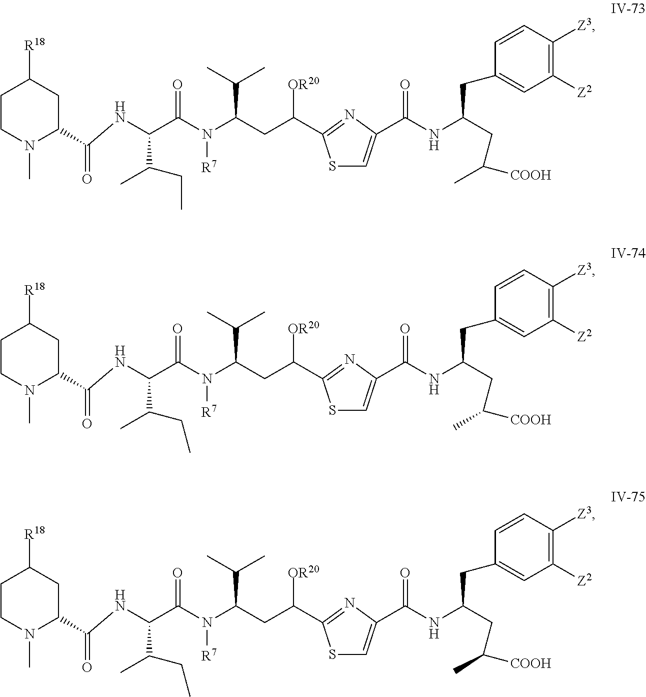

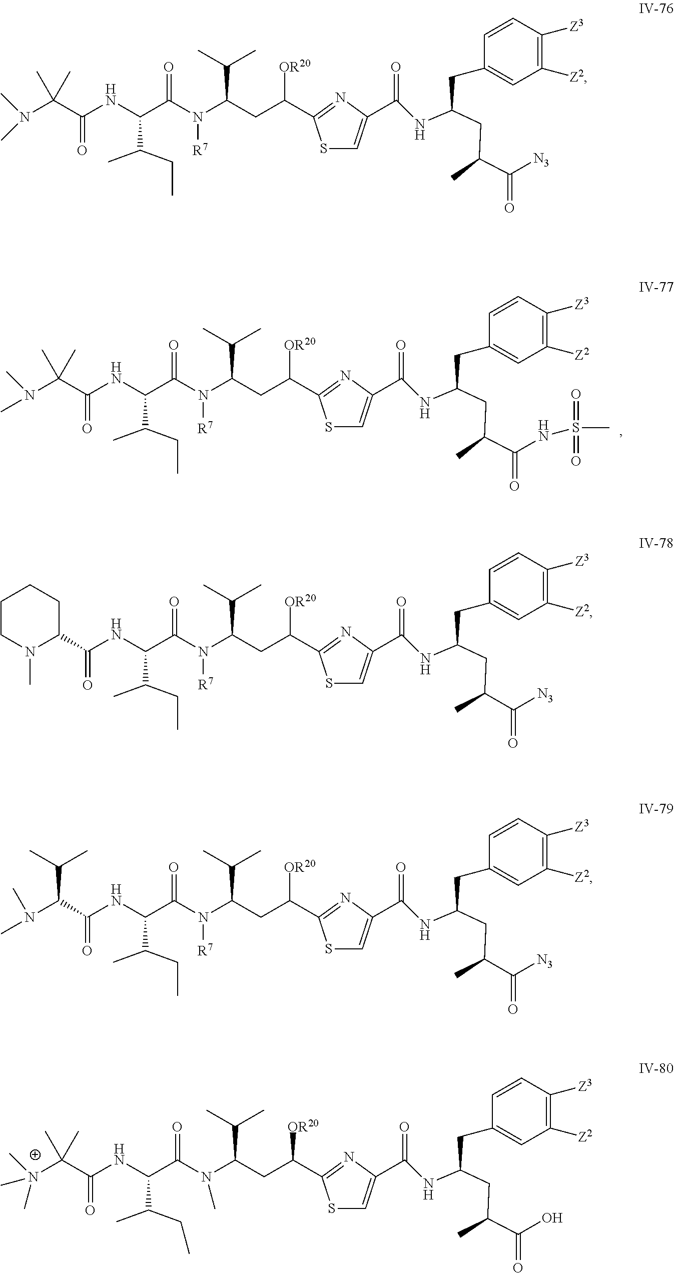

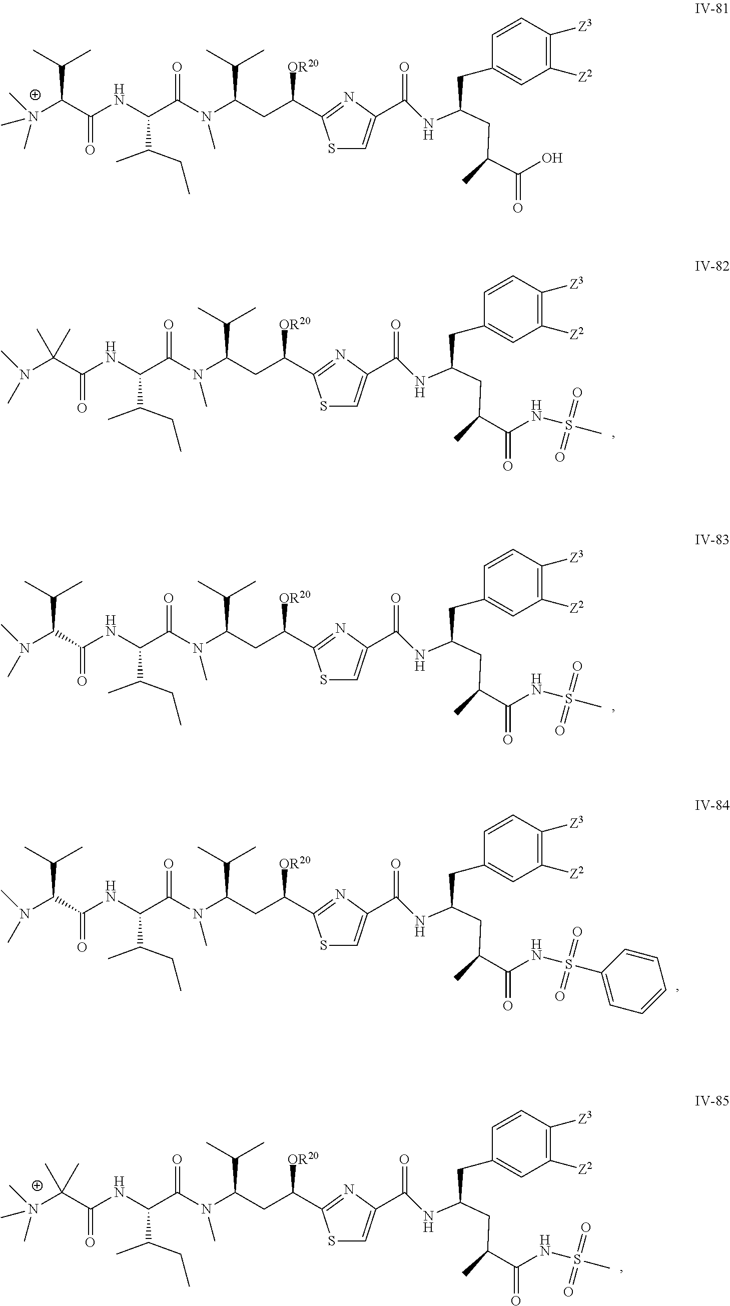

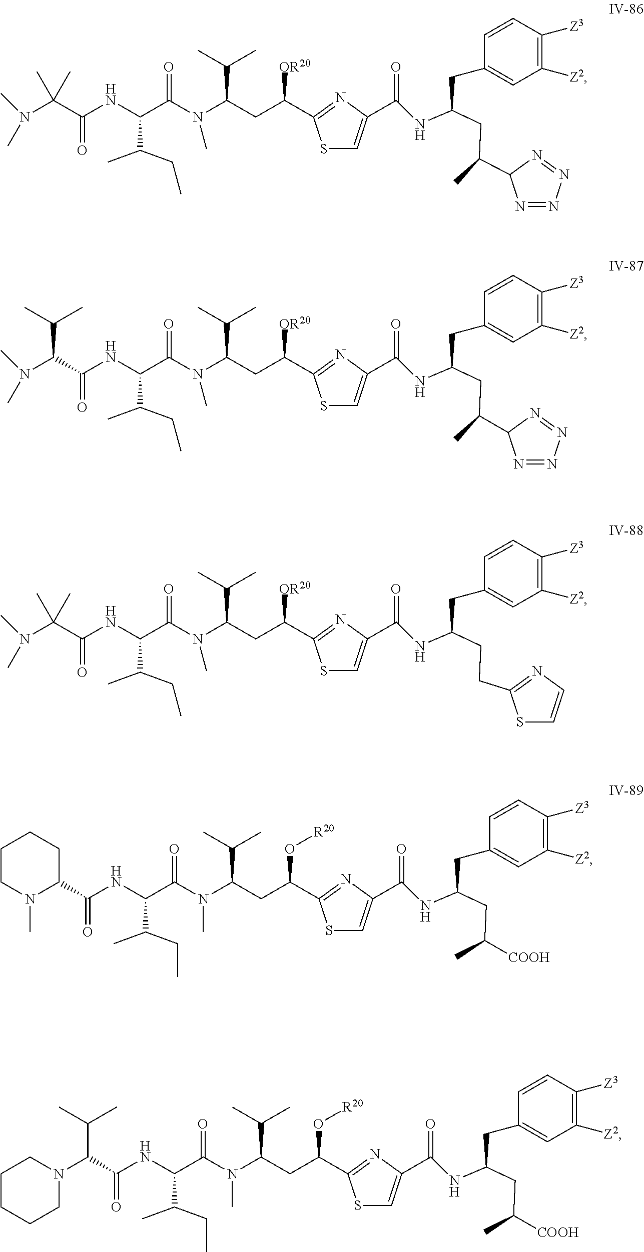

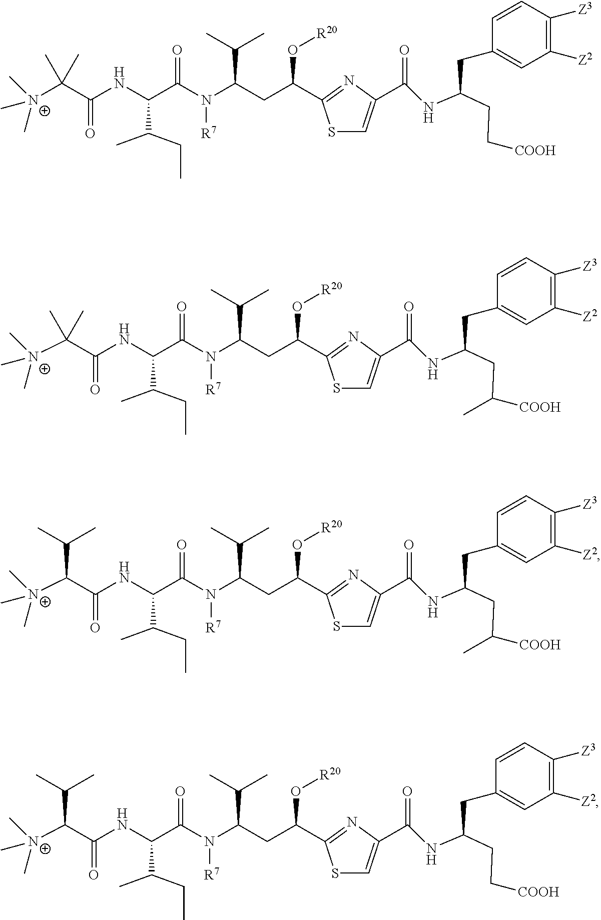

wherein R²⁰ is H; C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); or 1-8 carbon atoms of carboxylate, esters, ether, or amide; or 1˜8 amino acids; or polyethyleneoxy unit of formula (OCH₂CH₂)ₚ or (OCH₂CH(CH₃))ₚ, wherein p is an integer from 0 to about 1000; or R²⁰ is absent and the oxygen forms a ketone, or combination above thereof, Z³ and Z³ are independently H, OH, NH₂, O, NH, COOH, COO, C(O), C(O), C(O)NH, C(O)NH₂, R¹⁸, OCH₂OP(O)(OR¹⁸)₂, OC(O)OP(O)(OR¹⁸)₂, OPO(OR¹⁸)₂, NHPO(OR¹⁸)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁹ is H, OH, NH₂, OSO₂(OR¹⁸), XCH₂OP(O)(OR¹⁸)₂, XPO(OR¹⁸)₂, XC(O)OP(O)(OR¹⁸)₂, XC(O)R¹⁸, XC(O)NHR¹⁸, C₁˜C₈ alkyl or carboylate; C₂˜C₈ alkenyl, alkynyl, alkylcycloalkyl, heterocycloalkyl; C₃˜C₈ aryl or alkylcarbonyl; or pharmaceutical salts; X is O, S, NH, NHNH, or CH₂; R⁷ is defined the same above; wherein the linkage sites, “ [symbol] ” in formula IV-01-IV-79 are the same indication according to formula (IV).

Calicheamicins and their related enediyne antibiotics that are described in: Nicolaou, K. C. et al, Science 1992, 256, 1172-1178; Proc. Natl. Acad. Sci USA. 1993, 90, 5881-8), U.S. Pat. Nos. 4,970,198; 5,053,394; 5,108,912; 5,264,586; 5,384,412; 5,606,040; 5,712,374; 5,714,586; 5,739,116; 5,770,701; 5,770,710; 5,773,001; 5,877,296; 6,015,562; 6,124,310; 8,153,768. Exemplary enediynes include, but are not limited to, calicheamicin, esperamicin, uncialamicin, dynemicin, and their derivatives. The structure of calicheamicins is preferred the following formula:

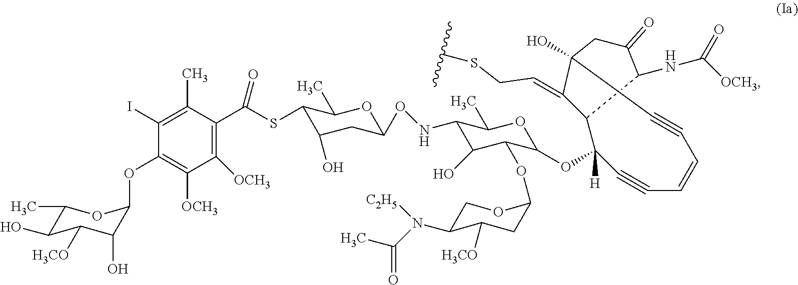 [SMILES: *SC/C=C1\C2[C@@H](OC3OC(C)C(NOC4CC(O)C(SC(=O)c5c(C)c(I)c(OC6OC(C)C(O)C(OC)C6O)c(OC)c5OC)C(C)O4)C(O)C3OC3CC(OC)C(N(CC)C(C)=O)CO3)C#C/C=C\C#C[C@]1(O)CC(=O)[C@@H]2NC(=O)OC]

or an isotope of a chemical element, or a pharmaceutically acceptable salt, hydrates, or hydrated salt; or a polymorphic crystalline structure; or an optical isomer, racemate, diastereomer or enantiomer thereof,
wherein [symbol] is the site linked to L₁ or L₂;
Geldanamycins are benzoquinone ansamycin antibiotic that bind to Hsp90 (Heat Shock Protein 90) and have been used antitumor drugs. Exemplary geldanamycins include, but are not limited to, 17-AAG (17-N-Allylamino-17-Demethoxygeldanamycin) and 17-DMAG (17-Dimethylaminoethylamino-17-demethoxygeldanamycin).

Maytansines or their derivatives maytansinoids inhibit cell proliferation by inhibiting the microtubules formation during mitosis through inhibition of polymerization of tubulin. See Remillard et al., Science 189:1002-1005 (1975). Exemplary maytansines and maytansinoids include, but are not limited to, mertansines (DM1, DM4), maytansinol and its derivatives as well as ansamitocin. Maytansinoidsare described in U.S. Pat. Nos. 4,256,746, 4,361,650, 4,307,016, 4,294,757, 4,294,757, 4,371,533, 4,424,219, 4,331,598, 4,450,254, 4,364,866, 4,313,946, 4,315,929 4,362,663, 4,322,348, 4,371,533, 4,424,219, 5,208,020, 5,416,064, 5,208,020; 5,416,064; 6,333,410; 6,441,163; 6,716,821, 7,276,497, 7,301,019, 7,303,749, 7,368,565, 7,411,063, 7,851,432, and 8,163,888. The structure of maytansinoids is preferred the following formula:

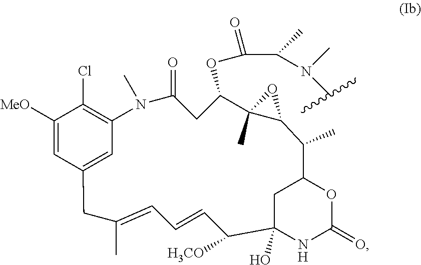

wherein [symbol] is the site linked to L₁ or L₂.

A camptothecin (CPTs) and its derivatives, which are topoisomerase inhibitors to prevent DNA re-ligation and therefore to causes DNA damage resulting in apoptosis, are described in: Shang, X. F. et al, Med Res Rev. 2018, 38(3):775-828; Botella, P. and Rivero-Buceta, E. J Control Release. 2017, 247: 28-54; Martino, E. et al, Bioorg Med Chem Lett. 2017, 27(4):701-707; Lu, A., et al, Acta Pharmacol Sin 2007, 28(2): 307-314. It includes SN-38, Topotecan, Irinotecan (CPT-11), Silatecan (DB-67, AR-67), Cositecan (BNP-1350), Etirinotecan, Exatecan, Lurtotecan, Gimatecan (ST1481), Belotecan (CKD-602), Rubitecan and several others (Shang, X. F. et al, Med Res Rev. 2018, 38(3):775-828). So far three CPT analogues, topotecan, irinotecan, and belotecan have been approved and are used in cancer chemotherapy (Palakurthi, S., Expert Opin Drug Deliv. 2015; 12(12):1911-21; Shang, X. F. et al, Med Res Rev. 2018, 38(3):775-828) and both SN-38 and Exatecan have been successfully used as payloads for ADC conjugates in the clinical trials (Ocean, A. J. et al, Cancer. 2017, 123(19): 3843-3854; Starodub, A. N., et al, Clin Cancer Res. 2015, 21(17): 3870-8; Cardillo, T. M., et al, Bioconjug Chem. 2015, 26(5): 919-31; Ogitani, Y. et al, Bioorg Med Chem Lett. 2016, 26(20): 5069-5072; Takegawa, N. et al, Int J Cancer. 2017 Oct. 15; 141(8):1682-1689. U.S. Pat. Nos. 7,591,994; 7,999,083, 8,080,250, 8,268,317; US patent applications 20130090458, 20140099258, 20150297748, 20160279259).

The structure of Camptothecin (CPT) is illustrated below formula:

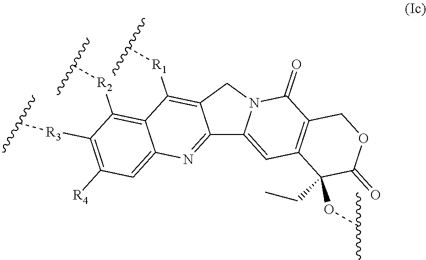

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R₁, R₂ and R₄ are independently selected from H, F, Cl, Br, CN, NO₂, C₁˜C₈ alkyl; O—C₁˜C₈ alkyl; NH—C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, carbonate, urea, or carbamate; R₃ is H, OH, NH₂, C₁˜C₈ alkyl; O—C₁˜C₈ alkyl; NH—C₁˜C₈ alkyl; C₂-C₈ of heteroalkyl, alkylcycloalkyl, heterocycloalkyl; or 2-8 carbon atoms of esters, ether, amide, carbonate, urea, or carbamate; or R₁R₂, R₂R₃ and R₃R₄ independently form a 5˜7 membered carbocyclic, heterocyclic, heterocycloalkyl, aromatic or heteroaromatic ring system; [symbol] is the site in the molecule that can be linked to L₁ or L₂.

The structures of camptothecins are preferred the following formula:

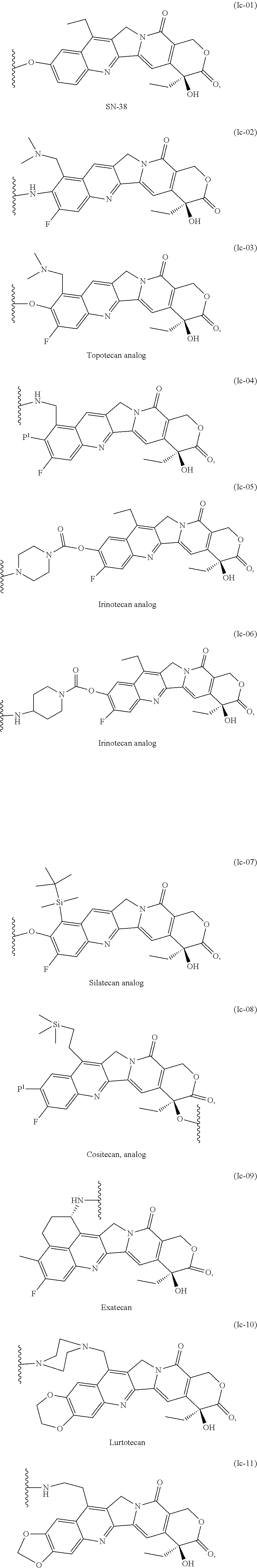

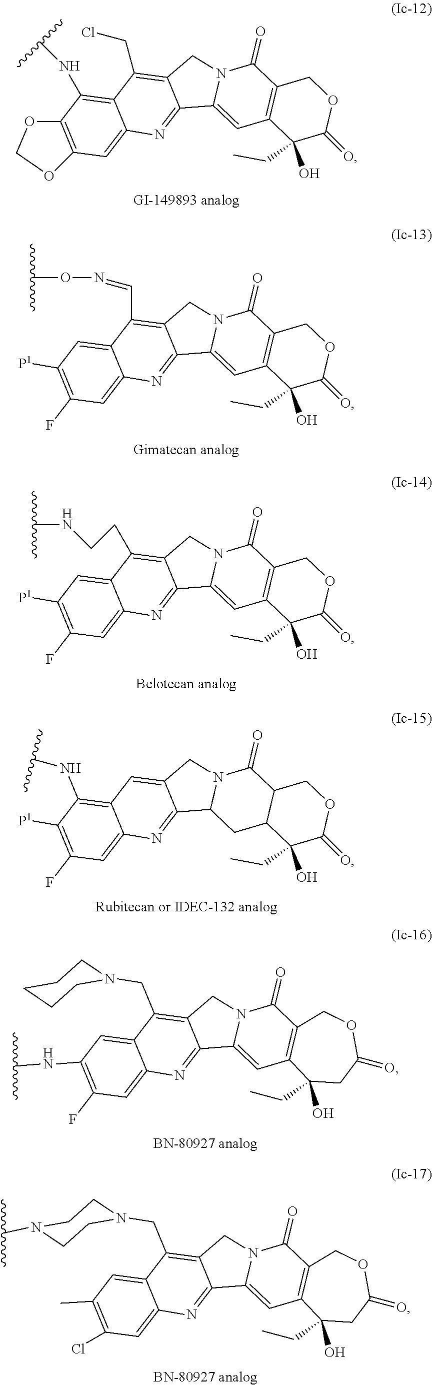

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the site linked to L₁ or L₂; P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH2N(C₂H₄)₂CH₃, O—(C₁-C₈ of linear or branched alkyl), C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸).

Combretastatins are natural phenols with vascular disruption properties in tumors. Exemplary combretastatins and their derivatives include, but are not limited to, combretastatin A-4 (CA-4), CA4-βGals, CA-4PD, CA4-NPs and ombrabulin.

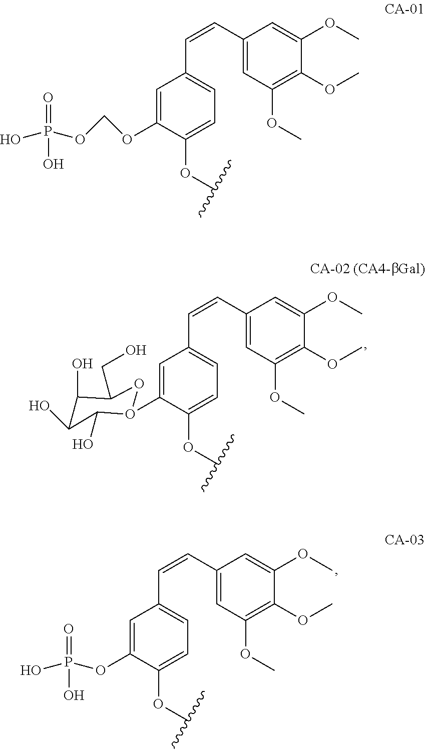

Taxanes, which includes Paclitaxel (Taxol), a cytotoxic natural product, and docetaxel (Taxotere), a semi-synthetic derivative, and their analogs which are preferred for conjugation are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5 (2004); U.S. Pat. Nos. 5,475,011 5,728,849, 5,811,452; 6,340,701; 6,372,738; 6,391,913, 6,436,931; 6,589,979; 6,596,757; 6,706,708; 7,008,942; 7,186,851; 7,217,819; 7,276,499; 7,598,290; and 7,667,054. The structures of taxanes are preferred the following formula:

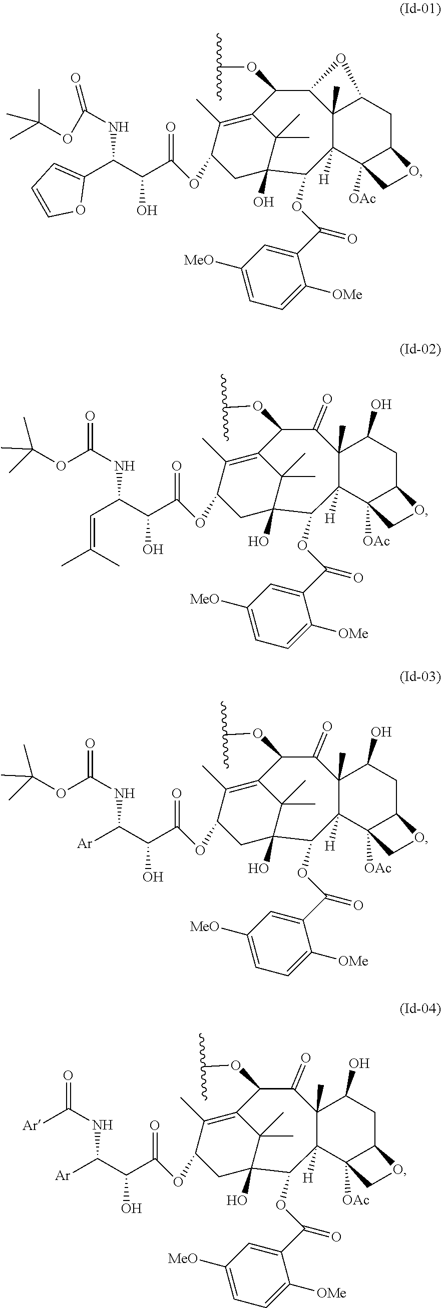

wherein [symbol] is the site linked to L₁ or L₂; Ar and Ar′ are independently aryl or heteroaryl.

Anthracyclines are mammalian DNA topoisomerases II inhibitors that are able to stabilize enzyme-DNA complexes wherein DNA strands are cut and covalently linked to the antibody. These anticancer agents maintain a prominent role in treating many forms of solid tumors and acute leukemias during the last several decades. However, anthracyclines cause cardiovascular morbidity and mortality (Sagi, J. C., et al, Pharmacogenomics. 2016, 17(9), 1075-87; McGowan, J. V., et al, Cardiovasc Drugs Ther. 2017, 31(1), 63-75). Thus, to enhance specific activity of such molecules while reducing the cardiotoxicity, researchers actively are using the conjugation of anthracyclines to a cell-binding antibody, or antibody molecule as a general approach for improving the therapeutic index of these drugs, (Mollaev, M. et al, Int J Pharm. 2018 Dec. 29. pii: S0378-5173(18) 30991-8; Rossin, R., et al, Bioconjug Chem. 2016, 27(7):1697-706; Dal Corso, A., et al, J Control Release. 2017, 264:211-218). Exemplary anthracyclines include, but are not limited to, daunorubicin, doxorubicin (i.e., adriamycin), epirubicin, idarubicin, valrubicin, and mitoxantrone. The structures of anthracyclines used for the present application are preferred the following formula:

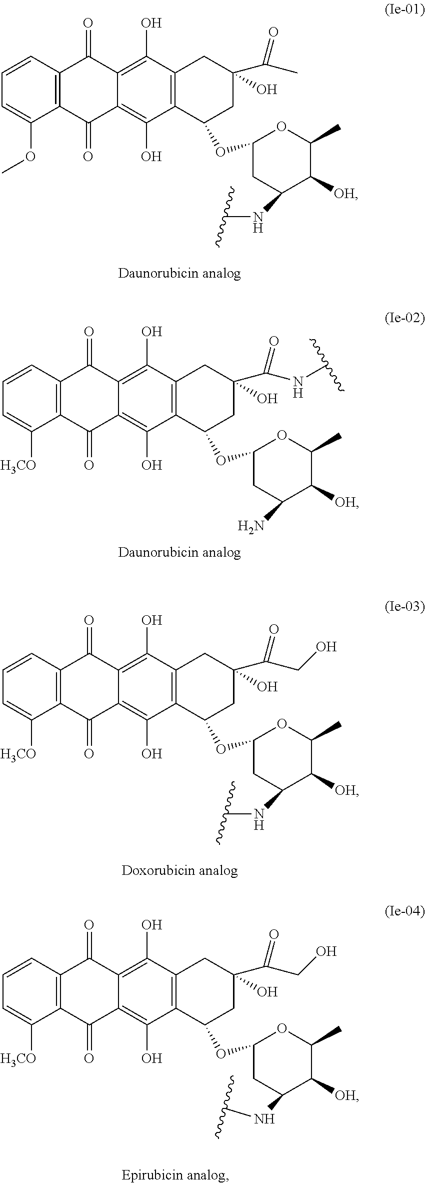

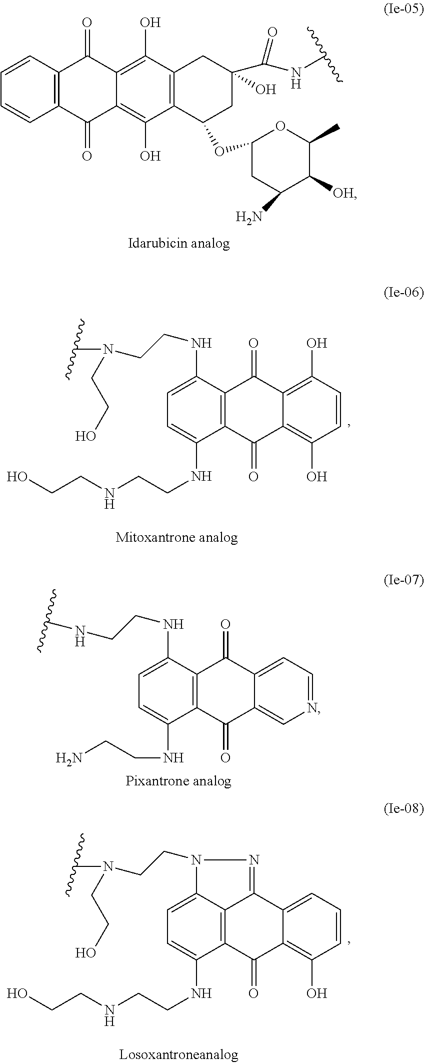

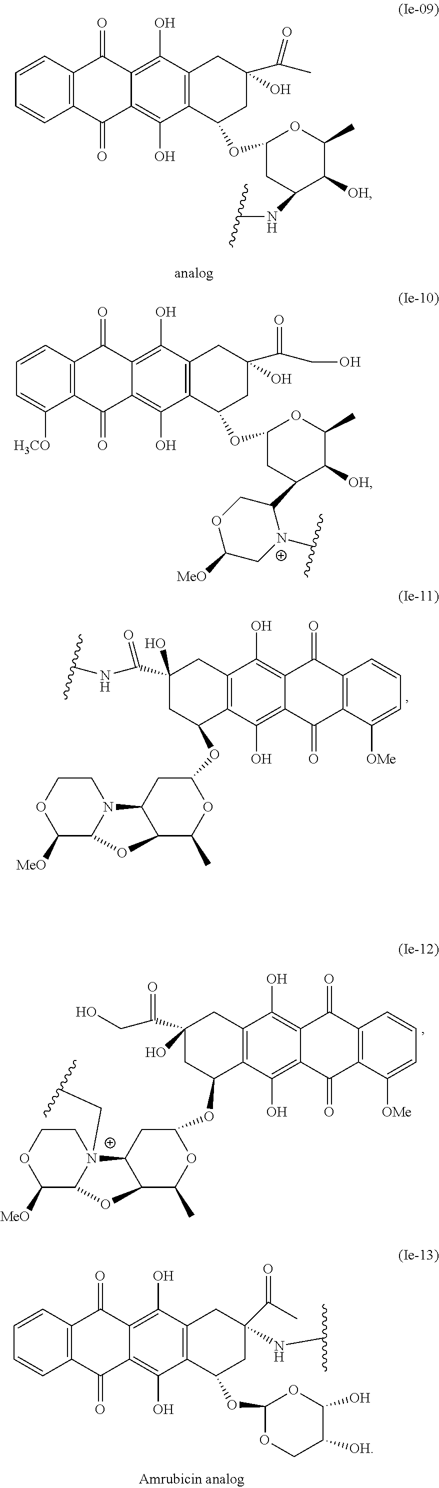

wherein [symbol] is the site that links to L₁ or L₂.

Vinca alkaloids are a set of anti-mitotic and anti-microtubule alkaloid agents that work by inhibiting the ability of cancer cells to divide. Vinca alkaloids include vinblastine, vincristine, vindesine, leurosine, vinorelbine, catharanthine, vindoline, vincaminol, vineridine, minovincine, methoxyminovincine, minovincinine, vincadifformine, desoxyvincaminol, vincamajine, vincamine, vinpocetine, and vinburnine. The structures of vinca alkaloids are preferred vinblastine, vincristine having the following formula:

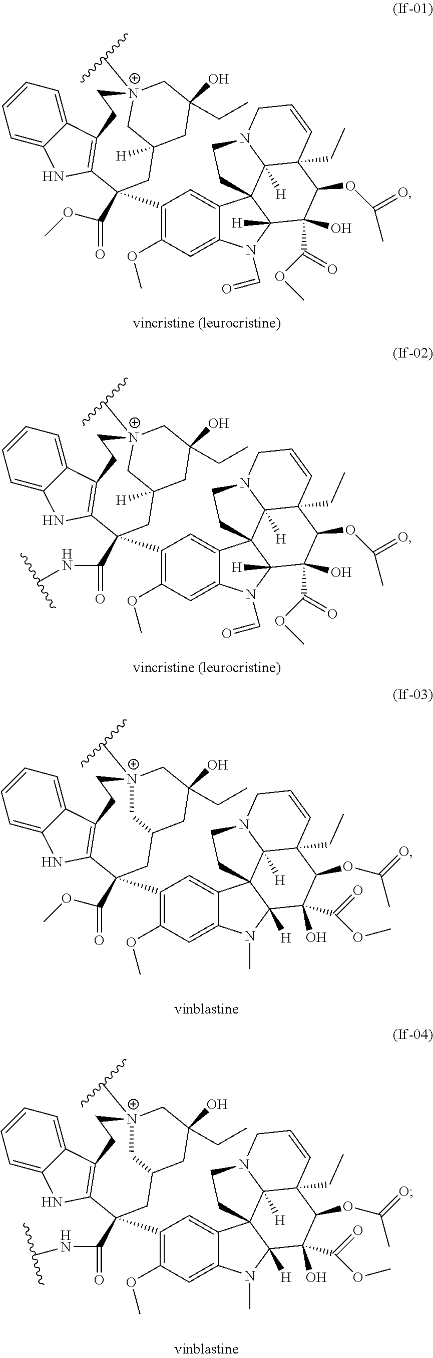

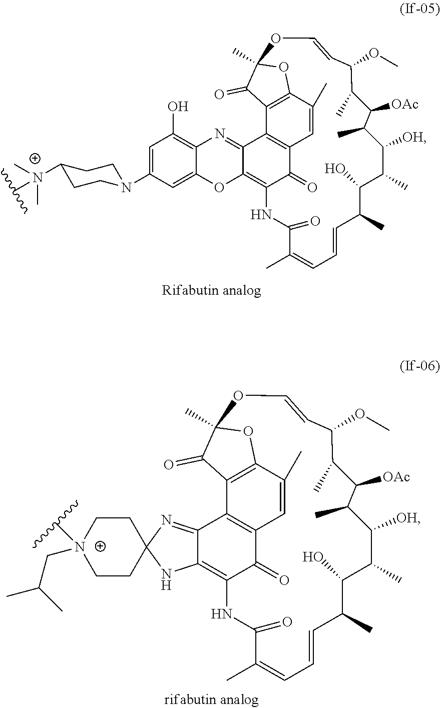

wherein [symbol] is the site linked to L₁ or L₂;

Dolastatins and their peptidic analogs and derivatives, auristatins, are highly potent antimitotic agents that have been shown to have anticancer and antifungal activity. See, e.g., U.S. Pat. No. 5,663,149 and Pettit et al., Antimicrob. Agents Chemother. 42:2961-2965, 1998. Exemplary dolastatins and auristatins include, but are not limited to, dolastatin 10, auristatin E (AL), auristatin EB (ALB), auristatin EFP (ALFP), MM4AD (Monomethyl Auristatin D or monomethyl dolastatin 10), MM4AF (Monomethyl Auristatin F or N-methylvaline-valine-dolaisoleuine-dolaproine-phenylalanine), MVMAL (Monomethyl Auristatin E or N-methylvaline-valine-dolaisoleuine-dolaproine-norephedrine), 5-benzoylvaleric acid-AL ester (ALVB), Auristatin F phenylene diamine (AFP) and other novel auristatins. The auristatins are described in Int. J. Oncol. 15: 367-72 (1999); Molecular Cancer Therapeutics, vol. 3, No. 8, pp. 921-32 (2004); U.S. Application Nos. 11134826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. The structures of auristatin analogs are preferred the following formula (Ih-01), (Ih-02), (Ih-03), (Ih-04), (Ih-05), (Ih-06), (Ih-07), (Ih-08), (Ih-09), (Ih-10), and (Ih-11):

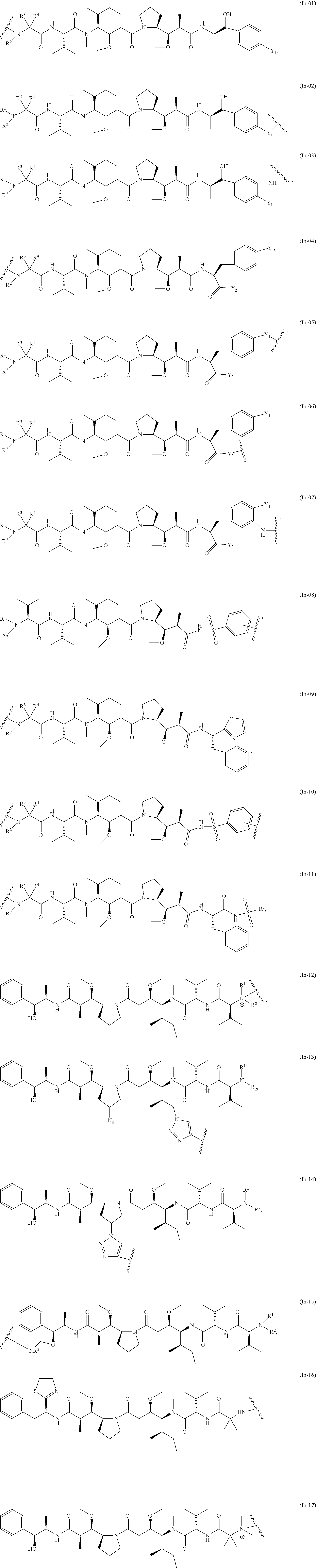

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 1000. The two Rs: R¹R², R²R³, R¹R³ or R³R⁴ together can form 3˜8 member cyclic ring of alkyl, aryl, heteroaryl, heteroalkyl, or alkylcycloalkyl group; Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ” (that links to L₁ and/or L₂ independently); or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-5000; R₁, R₂, R₃, R₄, R₅, R₅′, Z₁, Z₂, and n are defined the same above.

Hemiasterlin and its analogues (e.g., HTI-286) bind to the tubulin, disrupt normal microtubule dynamics, and, at stoichiometric amounts, depolymerize microtubules. The structure of maytansinoids is preferred the following formula:

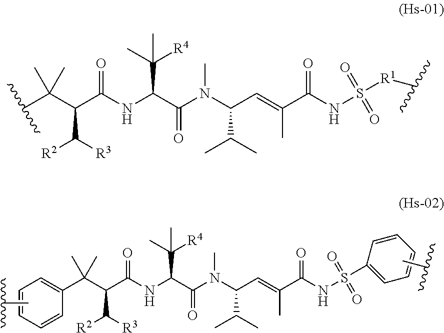

wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; In addition, R²R³ can form 3˜8 member cyclic ring of alkyl, aryl, heteroaryl, heteroalkyl, or alkylcycloalkyl group.

Eribulin which is binding predominantly to a small number of high affinity sites at the plus ends of existing microtubules has both cytotoxic and non-cytotoxic mechanisms of action. Its cytotoxic effects are related to its antimitotic activities, wherein apoptosis of cancer cells is induced following prolonged and irreversible mitotic blockade (Kuznetsov, G. et al, Cancer Research. 2004, 64 (16): 5760-6.; Towle, M. J, et al, Cancer Research. 2010, 71 (2): 496-505). In addition to its cytotoxic, antimitotic-based mechanisms, preclinical studies in human breast cancer models have shown that eribulin also exerts complex effects on the biology of surviving cancer cells and residual tumors that appear unrelated to its antimitotic effects. Eribulin has been approved by US FDA for the treatment of metastatic breast cancer who have received at least two prior chemotherapy regimens for late-stage disease, including both anthracycline- and taxane-based chemotherapies, as well as for the treatment of liposarcoma (a specific type of soft tissue sarcoma) that cannot be removed by surgery (unresectable) or is advanced (metastatic). Eribulin has been used as payload for ADC conjugates (US20170252458). The structure of Eribulin is preferred the following formula, Eb01:

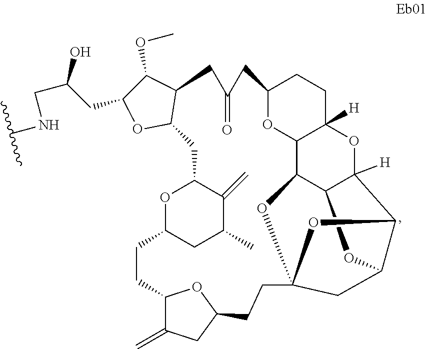 [SMILES: *NC[C@@H](O)C[C@H]1O[C@H]2C[C@H]3O[C@@H](CC[C@@H]4O[C@@H](CC[C@@]56C[C@H]7O[C@@H]8C(O[C@H]9CC[C@H](CC(=O)C[C@@H]2[C@H]1OC)OC9[C@@H]8O5)[C@H]7O6)CC4=C)C[C@@H](C)C3=C]

[symbol] is a linkage site that links to L₁ and/or L₂ independently;

An Inhibitor of nicotinamide phosphoribosyltransferases (NAMPT) can be an interesting ADC payload due to their unique mechanisms of high potent activity (Sampath D, et al, Pharmacol Ther 2015; 151, 16-31). NAMPT regulates nicotinamide adenine dinucleotide (NAD) levels in cells wherein NAD plays as an essential redox cofactor to support energy and anabolic metabolism. NAD has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that can be released from cells spontaneously and by regulated mechanisms (Smyth L. M, et al, J. Biol. Chem. 2004, 279 (47), 48893-903; Billington R. A, et al, Mol Med. 2006, 12, 324-7), and can therefore have important extracellular roles (Billington R. A, et al, Mol Med. 2006, 12, 324-7). When inhibitors of NAMPT present, NAD levels decline below the level needed for metabolism resulting in energy crisis and therefore cell death. So far, clinical NAMPT inhibitor candidates FK-866, CHS-828, and GMX-1777 advanced to clinical trials but each encountered dose-limiting toxicities prior to any objective responses (Holen K., et al, Invest New Drugs 2008, 26, 45-51; Hovstadius, P., et al, Clin Cancer Res 2002, 8, 2843-50; Pishvaian, M. J., et al, J Clin Oncol 2009, 27, 3581). Thus using ADCs for targeting delivery of NAMPT inhibitors might circumvent the systemic toxicities to achieve much broader therapeutic index. The structures of NAMPT inhibitors are preferred the following formula, NP01, NP02, NP03, NP04, NP05, NP06, NP07, NP08, and NP09:

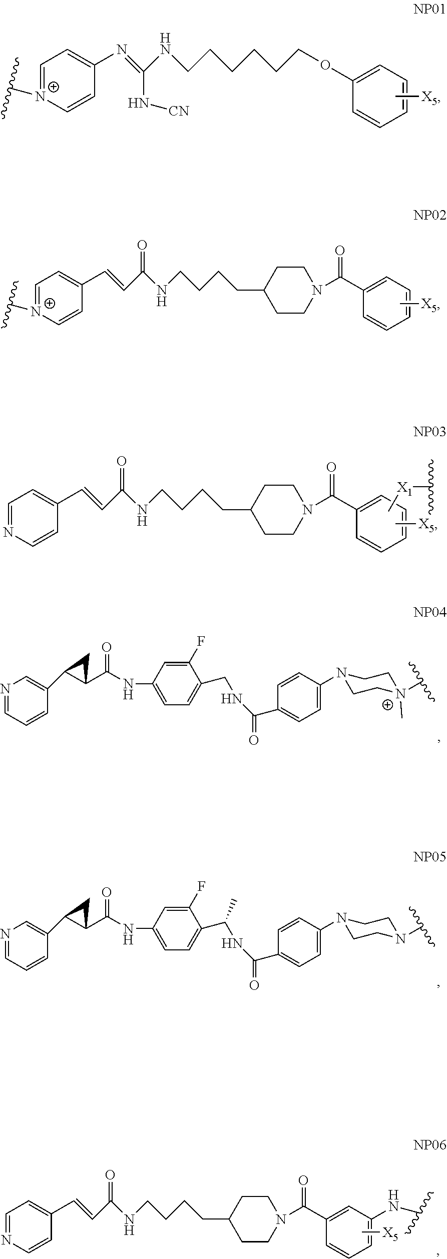

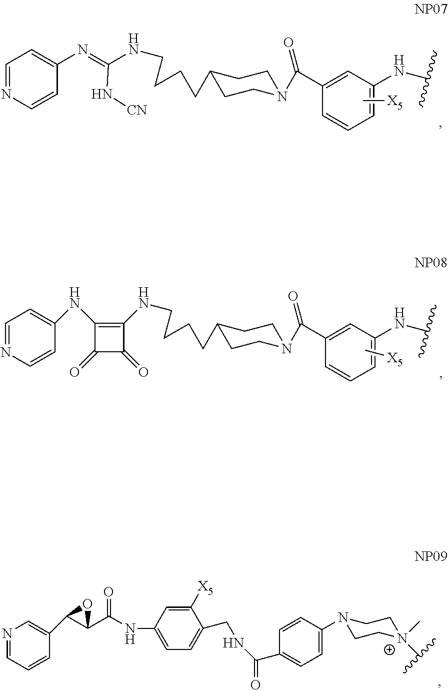

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR₁, OCOR₁, NHCOR₁.

A benzodiazepine dimer and its analogs: (e. g. a dimer of pyrrolobenzodiazepine (PBD) or (tomaymycin), a dimer of indolinobenzodiazepine (IGN), a dimer of imidazobenzothiadia-zepine, or a dimer of oxazolidinobenzodiazepines) are anti-tumor agents that contain one or more immune functional groups, or their equivalents, that bind to duplex DNA. PBD and IGN molecules are based on the natural product athramycin, and interact with DNA in a sequence-selective manner, with a preference for purine-guanine-purine sequences. The preferred benzodiazepine dimers according to the present invention are exampled in: U.S. Pat. Nos. 8,163,736; 8,153,627; 8,034,808; 7,834,005; 7,741,319; 7,704,924; 7,691,848; 7,678,787; 7,612,062; 7,608,615; 7,557,099; 7,528,128; 7,528,126; 7,511,032; 7,429,658; 7,407,951; 7,326,700; 7,312,210; 7,265,105; 7,202,239; 7,189,710; 7,173,026; 7,109,193; 7,067,511; 7,064,120; 7,056,913; 7,049,311; 7,022,699; 7,015,215; 6,979,684; 6,951,853; 6,884,799; 6,800,622; 6,747,144; 6,660,856; 6,608,192; 6,562,806; 6,977,254; 6,951,853; 6,909,006; 6,344,451; 5,880,122; 4,935,362; 4,764,616; 4,761,412; 4,723,007; 4,723,003; 4,683,230; 4,663,453; 4,508,647; 4,464,467; 4,427,587; 4,000,304; US patent appl. 20100203007, 20100316656, 20030195196. Examples of the structures of the conjugate of the antibody-benzodiazepine dimers are illustrated below PB01, PB02, PB03, PB04, PB05, PB06, PB07, PB08, PB09, PB10, PB11, PB12, PB13, PB14, PB15, PB16, PB17, PB18, PB19, PB20:

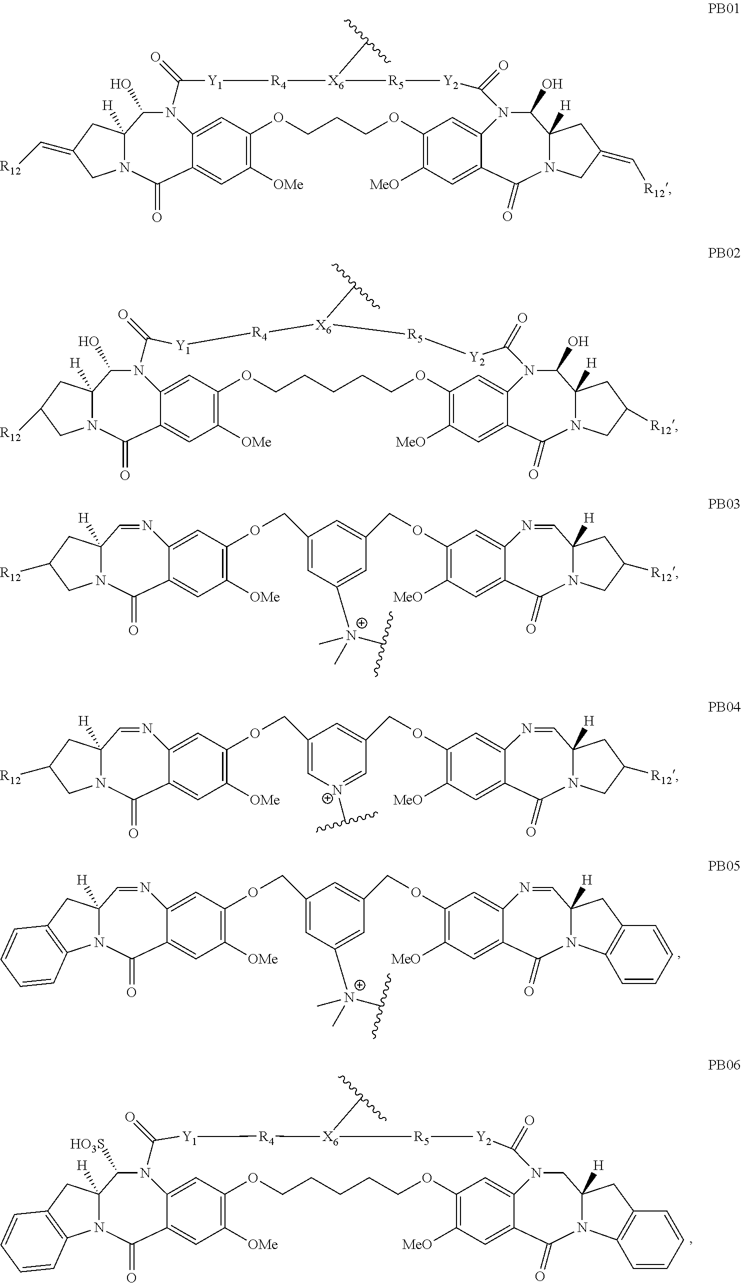

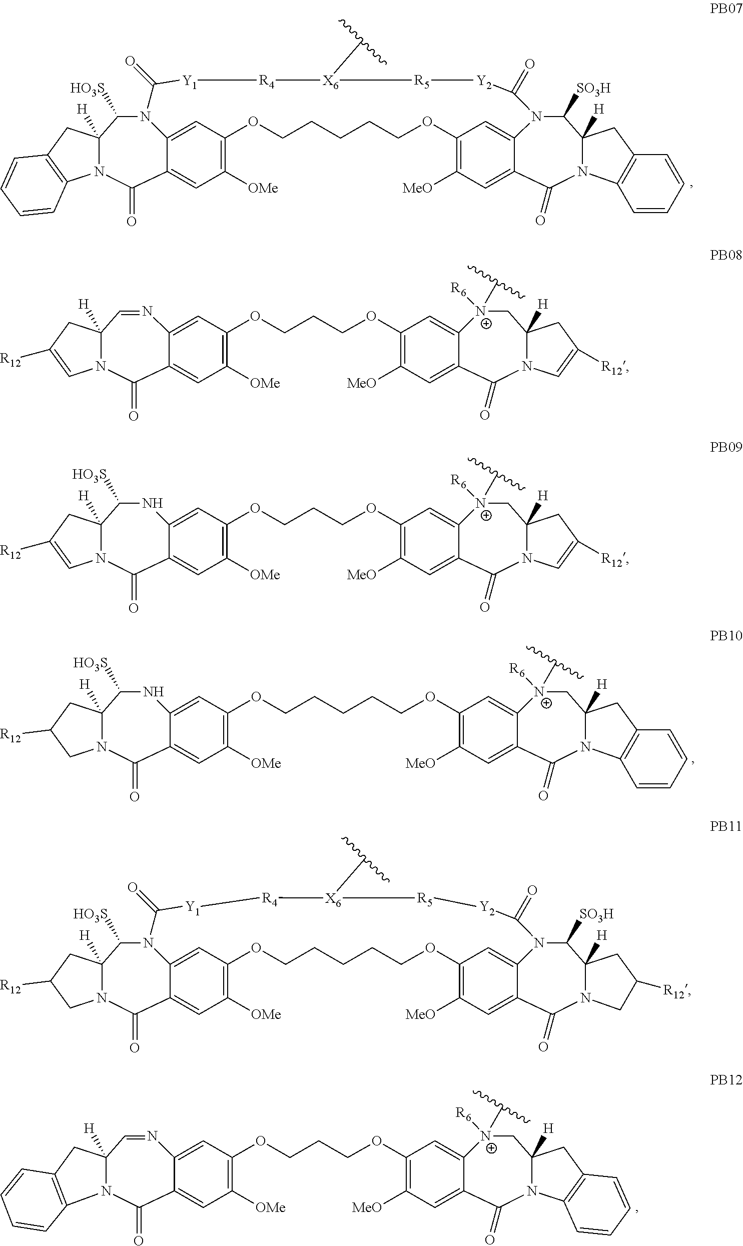

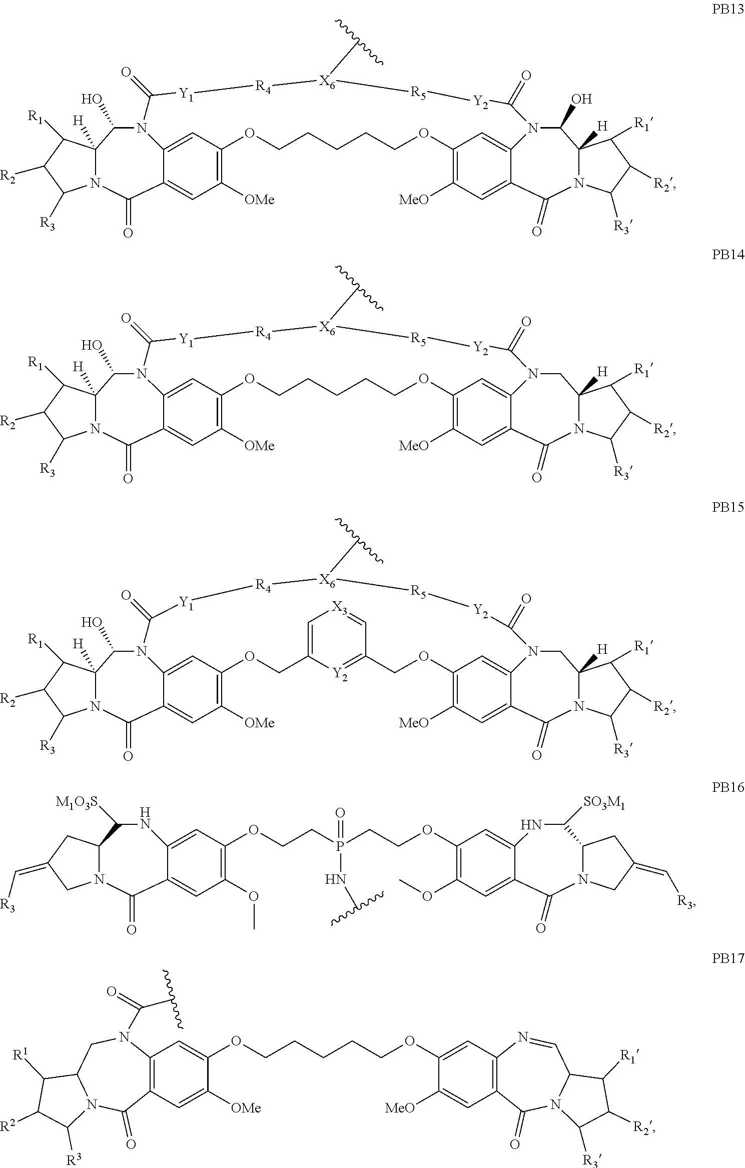

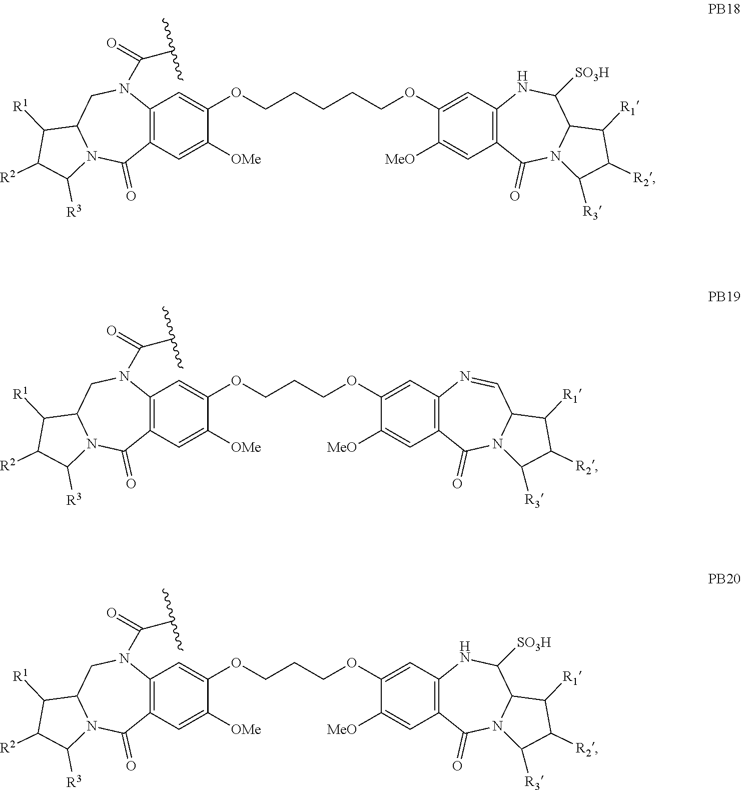

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁, Y₂, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R¹, R², R³, R^{1′}, R^{2′}, and R^{3′} are independently H; F; Cl; ═O; ═S; ═CH₂; ═CH—R₁, OH; SH; C₁-C₈ linear or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amines (NHR₅, NR₅R₅′), heterocycloalkyl, or acyloxylamines (—C(O)NHOH, —ONHC(O)R₅); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000. The two Rs: R¹R², R²R³, R^{1′}R^{2′}, or R^{2′}R^{3′}, can independently form 3-8 member cyclic ring of alkyl, aryl, heteroaryl, heteroalkyl, or alkylcycloalkyl group; X₃ and Y₃ are independently N, NH, CH₂ or CR₅, or one of X₃ and Y₃ can be absent; wherein R₁, and R₂ are C₁-C₈ linear or branched alkyl, heteroalkyl; C₃-C₈ aryl, heteroaryl, alkylcycloalkyl, acyloxyl, alkylaryl, alkylaryloxyl, alkylarylamino, alkylarylthiol; or 1-6 the same or different sequence of amino acid/peptides (Aa)r, r=1-6; wherein R₄, R₅, R₅′, R₆, R₁₂ and R₁₂′ are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃; X₆ is CH, N, P(O)NH, P(O)NR₁, CHC(O)NH, C₃-C₈aryl, heteroaryl, alkylcycloalkyl, acyloxyl, alkylaryl, alkylaryloxyl, alkylarylamino, or an Aa (amino acid, is preferably selected from Lys, Phe, Asp, Glu, Ser, Thr, His, Cys, Tyr, Trp, Gln, Asn, Arg); “ [symbol] ” is defined the same above.

An CC-1065 analog and doucarmycin analogs are also preferred to be used for a conjugate of the present process invention. The examples of the CC-1065 analogues and doucarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5,786,486, 5,789,650, 5,814,318, 5,846,545, 5,874,299, 5,877,296, 5,877,397, 5,885,793, 5,939,598, 5,962,216, 5,969,108, 5,985,908, 6,060,608, 6,066,742, 6,075,181, 6,103,236, 6,114,598, 6,130,237, 6,132,722, 6,143,901, 6,150,584, 6,162,963, 6,172,197, 6,180,370, 6,194,612, 6,214,345, 6,262,271, 6,281,354, 6,310,209, 6,329,497, 6,342,480, 6,486,326, 6,512,101, 6,521,404, 6,534,660, 6,544,731, 6,548,530, 6,555,313, 6,555,693, 6,566,336, 6,586,618, 6,593,081, 6,630,579, 6,756,397, 6,759,509, 6,762,179, 6,884,869, 6,897,034, 6,946,455, 7,049,316, 7,087,600, 7,091,186, 7,115,573, 7,129,261, 7,214,663, 7,223,837, 7,304,032, 7,329,507, 7,329,760, 7,388,026, 7,655,660, 7,655,661, 7,906,545, and 8,012,978. Examples of the structures of the conjugate of the antibody-CC-1065 analogs via the linker of the patent are illustrated below CC01, CC02, CC03, CC04, CC05, CC06 and CC07:

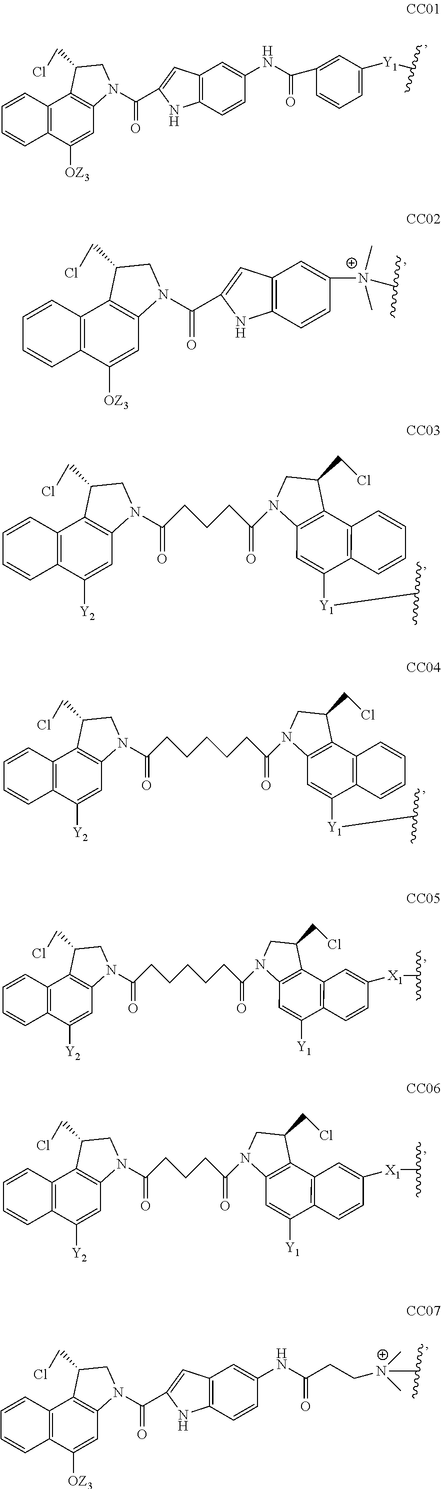

wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are defined the same above;

An amatoxin and its analogs which are a subgroup of at least ten toxic compounds originally found in several genera of poisonous mushrooms, most notably Amanita phalloides and several other mushroom species, are also preferred for conjugation of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American 232 (3): 90-101; H. E. Hallen, et al 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15(16): 3480-4; Fiume, L., Curr Probl Clin Biochem, 1977, 7: 23-8; Karlson-Stiber C, Persson H. 2003, Toxicon 42(4): 339-49; Chafin, D. R., Guo, H. & Price, D. H. 1995 J. Biol. Chem. 270 (32): 19114-19; Wieland (1983) Int. J. Pept. Protein Res. 22(3):257-76.). Amatoxins can be produced from collected Amanita phalloides mushrooms (Yocum, R. R. 1978 Biochemistry 17(18): 3786-9; Zhang, P. et al, 2005, FEMS Microbiol. Lett. 252(2), 223-8), or from fermentation using a basidiomycete (Muraoka, S. and Shinozawa T., 2000 J. Biosci. Bioeng. 89(1): 73-6) or from fermentation using A. fissa (Guo, X. W., et al, 2006 Wei Sheng Wu Xue Bao 46(3): 373-8), or from culturing Galerina fasciculata or Galerina helvoliceps, a strain belonging to the genus (WO/1990/009799, JP11137291). However, the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18(2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870(3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32(1): 9-20; G. Zanotti, T. et al, Int. J. Peptide Protein Res. 1989, 34, 222-8; Zanotti, G., et al., Int J Pept Protein Res, 1990. 35(3): 263-70; Mullersman, J. E. and J. F. Preston, 3rd, Int J Pept Protein Res, 1991. 37(6): 544-51; Mullersman, J. E., et al, Int J Pept Protein Res, 1991. 38(5): 409-16; Zanotti, G., et al, Int J Pept Protein Res, 1992. 40(6): 551-8; Schmitt, W. et al, J. Am. Chem. Soc. 1996, 118, 4380-7; Anderson, M. O., et al, J. Org. Chem., 2005, 70(12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88(5): 714-24; May, J. P., et al., Chemistry, 2008. 14(11): 3410-7; S. De Lamo Marin, et al, Eur. J. Org. Chem. 2010, 3985-9; Pousse, G., et al., Org Lett, 2010. 12(16): 3582-5; Luo, H., et al., Chem Biol, 2014. 21(12): 1610-7; Zhao, L., et al., Chembiochem, 2015. 16(10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243(130): 281-3; Bonetti, E., M. et al, Arch Toxicol, 1976. 35(1): p. 69-73; Davis, M. T., Preston, J. F. Science 1981, 213, 1385-1388; Preston, J. F., et al, Arch Biochem Biophys, 1981. 209(1): 63-71; H. Faulstich, et al, Biochemistry 1981, 20, 6498-504; Barak, L. S., et al., Proc Natl Acad Sci USA, 1981. 78(5): 3034-8; Faulstich, H. and L. Fiume, Methods Enzymol, 1985. 112: 225-37; Zhelev, Z., A. et al, Toxicon, 1987. 25(9): 981-7; Khalacheva, K., et al, Eksp Med Morfol, 1990. 29(3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J. E. and J. F. Preston, Biochem Cell Biol, 1991. 69(7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16(10): 1420-5; Zhou, B., et al., Biosens Bioelectron, 2015. 68: 189-96; WO2014/043403, US20150218220, EP 1661584). We have been working on the conjugation of amatoxins for a while. Examples of the structures of the amatoxins used for the present application are preferred the following structures of Am01, Am02, and Am03:

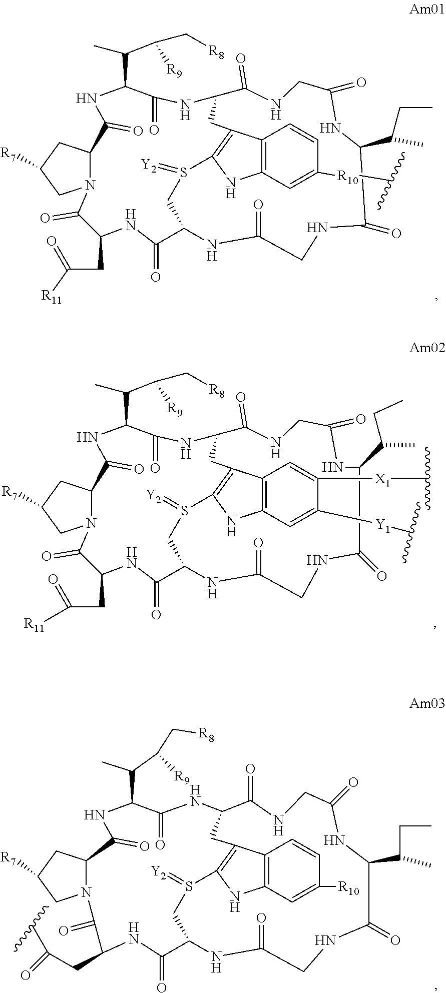

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NHNHC(O) and C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H2, wherein (Aa)ᵣ is 1-8 aminoacids; n and m₁ are independently 1-20; p is 1-5000; R₁, R² and Ar, are the same defined through out the application; “ [symbol] ” is defined the same above.

Spliceostatins and pladienolides are anti-tumor compounds which inhibit splicing and interacts with spliceosome, SF3b. Examples of spliceostatins include, but are not limited to, spliceostatin A, FR901464, and (2S, 3Z)-5-{[(2R, 3R, 5S, 6S)-6-{(2E, 4E)-5-[(3R, 4R, 5R, 7S)-7-(2-hydrazinyl-2-oxoethyl)-4-hydroxy-1, 6-dioxaspiro[2.5]oct-5-yl]-3-methylpenta-2, 4-dien-l-y-l}-2, 5-dimethyltetrahydro-2H-pyran-3-yl]amino}-5-oxopent-3-en-2-yl acetate having the core structure:

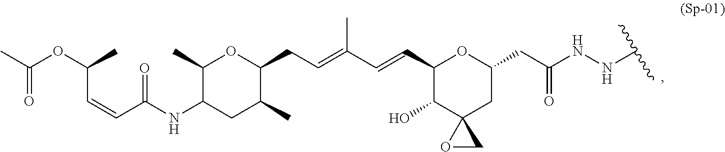 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1]

Examples of pladienolides include, but are not limited to, Pladienolide B, Pladienolide D, and E7107.

Protein kinase inhibitors that block the action of an enzyme to add a phosphate (PO₄) group to serine, threonine, or tyrosine amino acids on an antibody, and can modulate the protein function. The protein kinase inhibitors can be used to treat diseases due to hyperactive protein kinases (including mutant or overexpressed kinases) in cancer or to modulate cell functions to overcome other disease drivers. The structures of protein kinase inhibitors are preferred to selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, Vemurafenib, Entrectinib, Palbociclib, Ribociclib, Abemaciclib, Dacomitinib, Neratinib, Rociletinib (CO-1686), Osimertinib, AZD3759, Nazartinib (EGF816), having the following formula, PK01˜PK40:

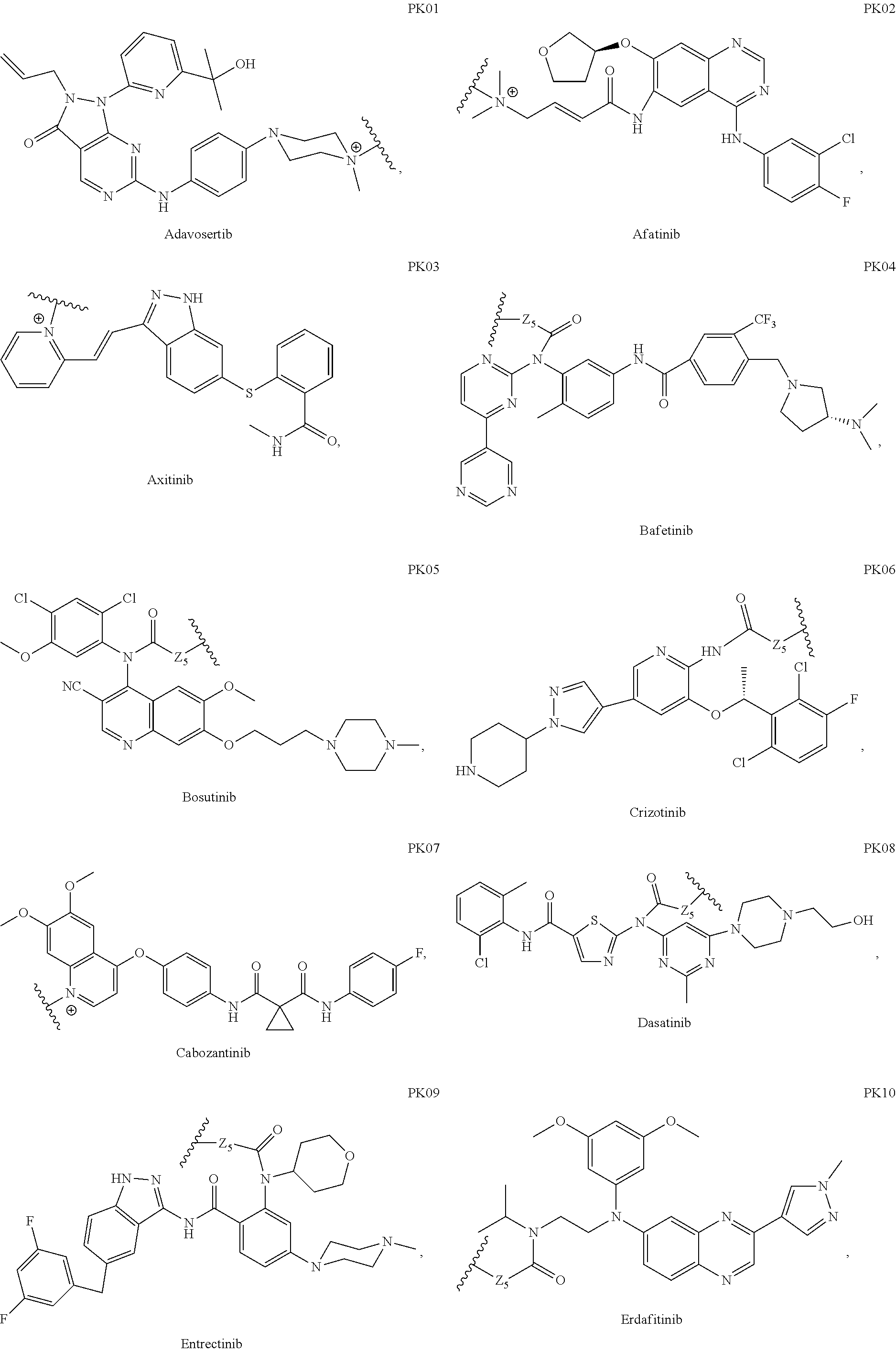

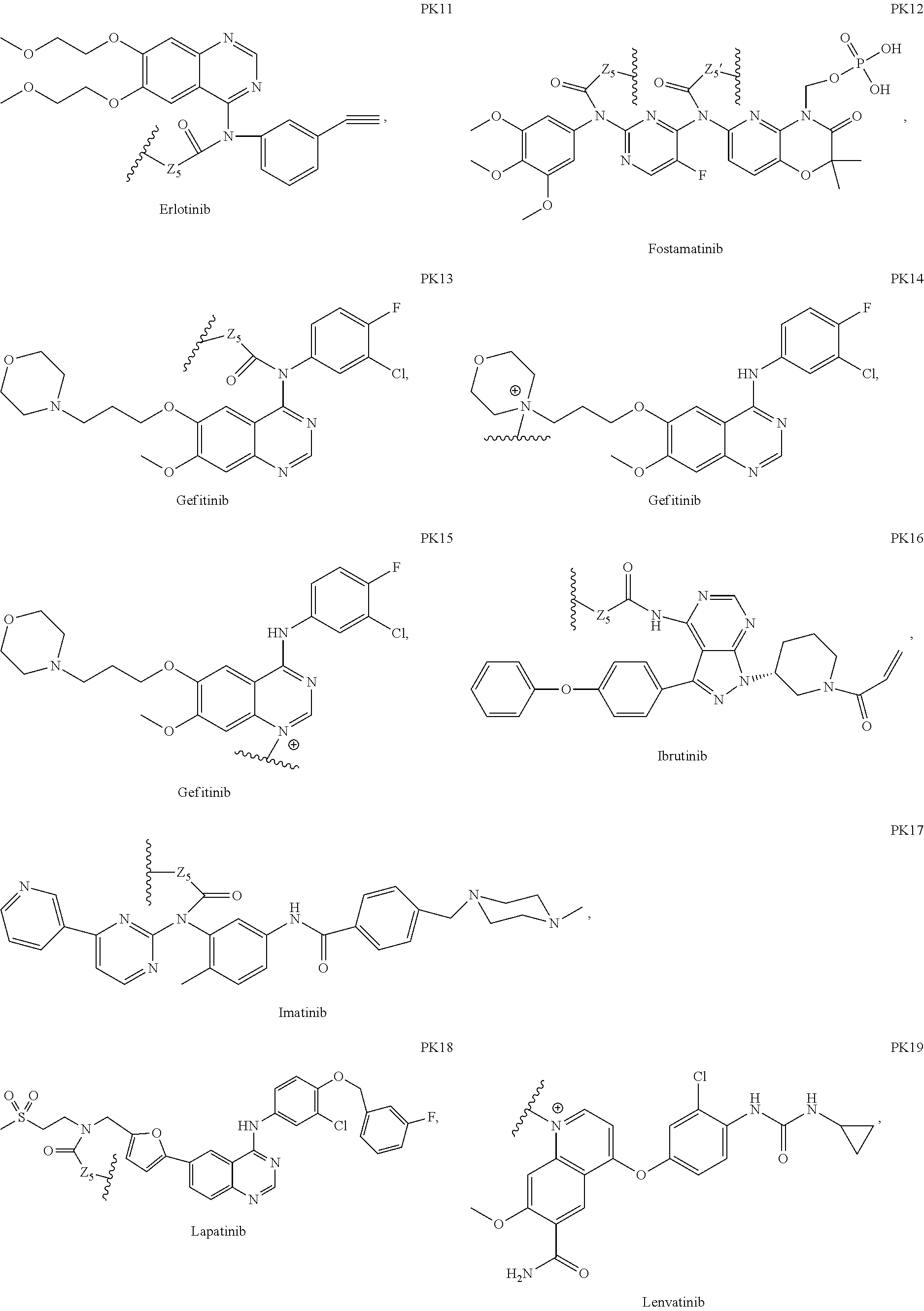

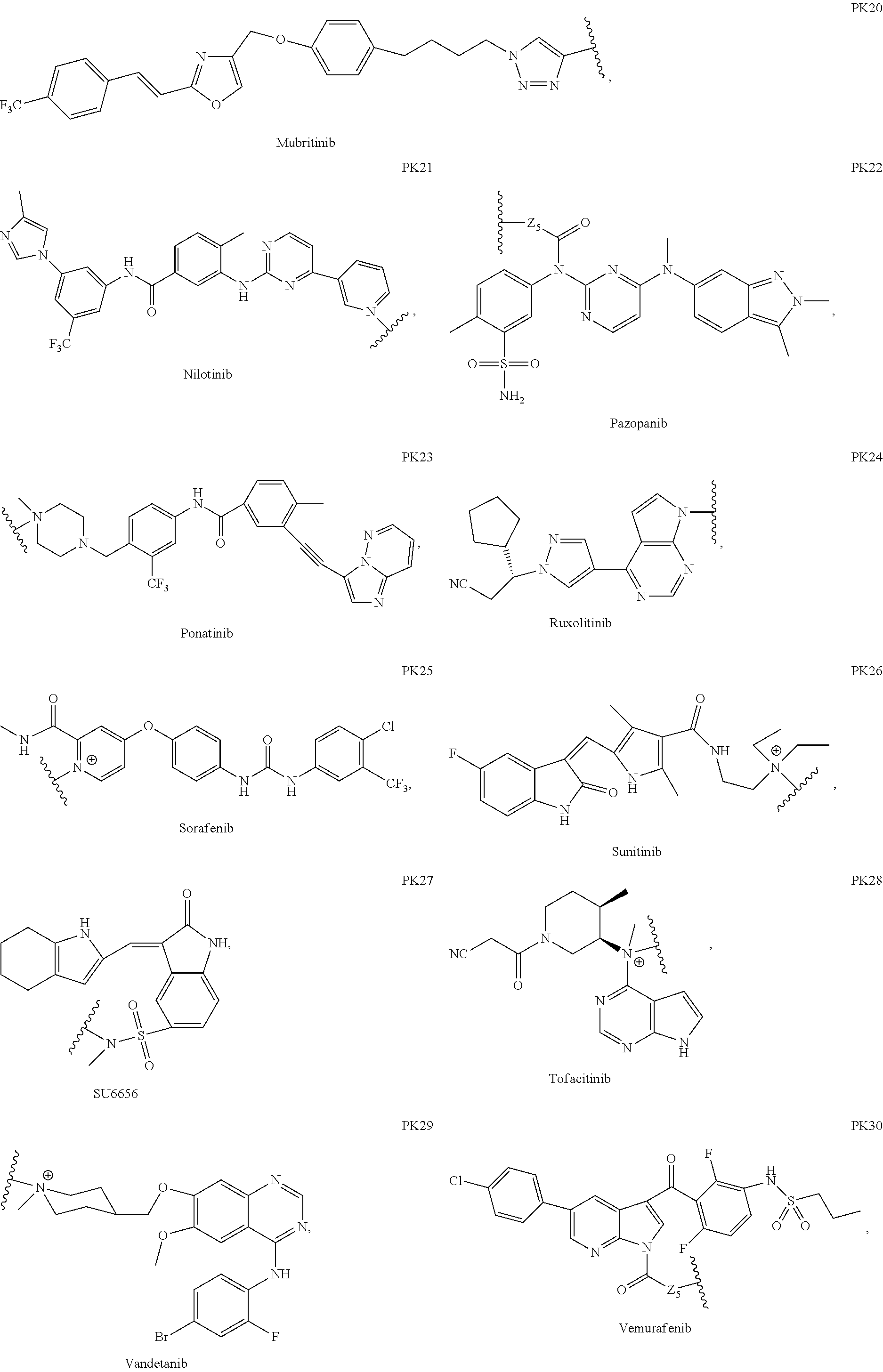

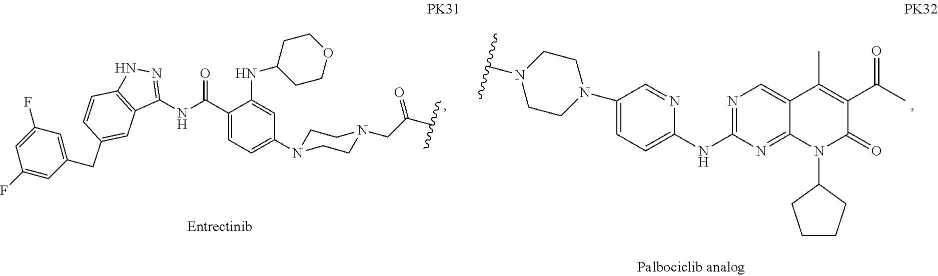

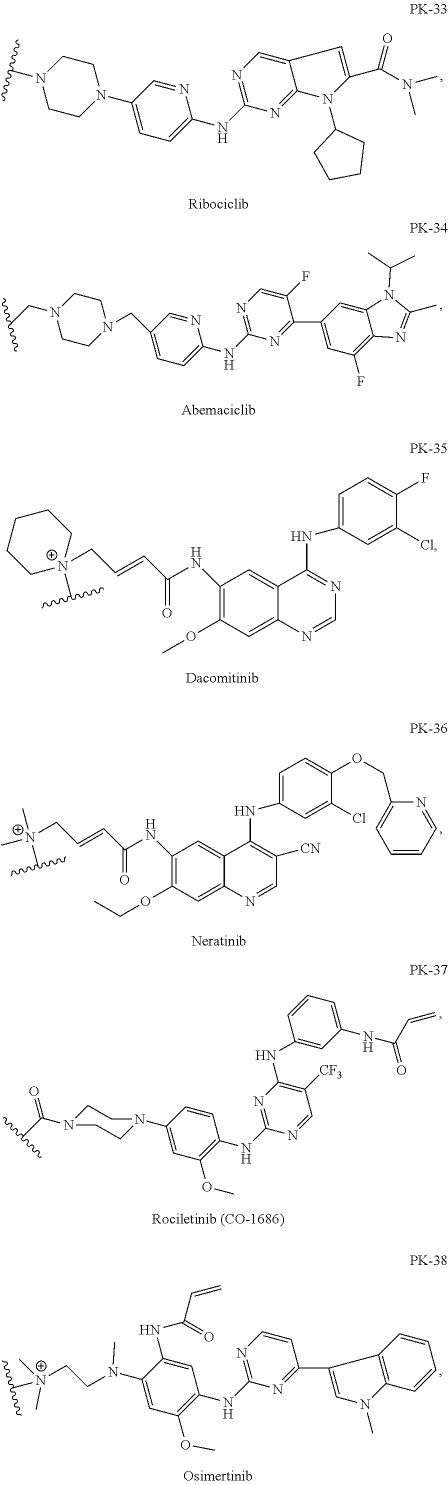

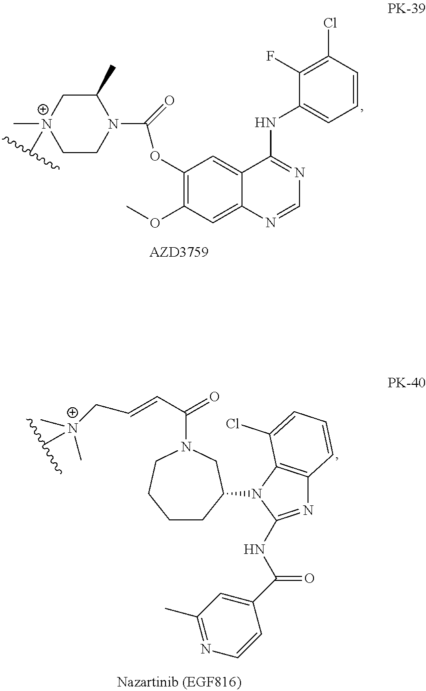

wherein Z₅ and Z₅′ are independently selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁.

A MEK inhibitor inhibits the mitogen-activated protein kinases MEK1 and/or MEK2 which is often overactive in some cancers. MEK inhibitors are especially used for treatment of BRAF-mutated melanoma, and KRAS/BRAF mutated colorectal cancer, breast cancer, and non-small cell lung cancer (NSCLC). MEK inhibitors are selected from PD0325901, selumetinib (AZD6244), cobimetinib (XL518), refametinib, trametinib (GSK1120212), pimasertib, Binimetinib (MEK162), AZD8330, RO4987655, RO5126766, WX-554, E6201, GDC-0623, PD-325901 and TAK-733. The preferred MEK inhibitors are selected from Trametinib (GSK1120212), Cobimetinib (XL518), Binimetinib (MEK162), selumetinib having the following formula:

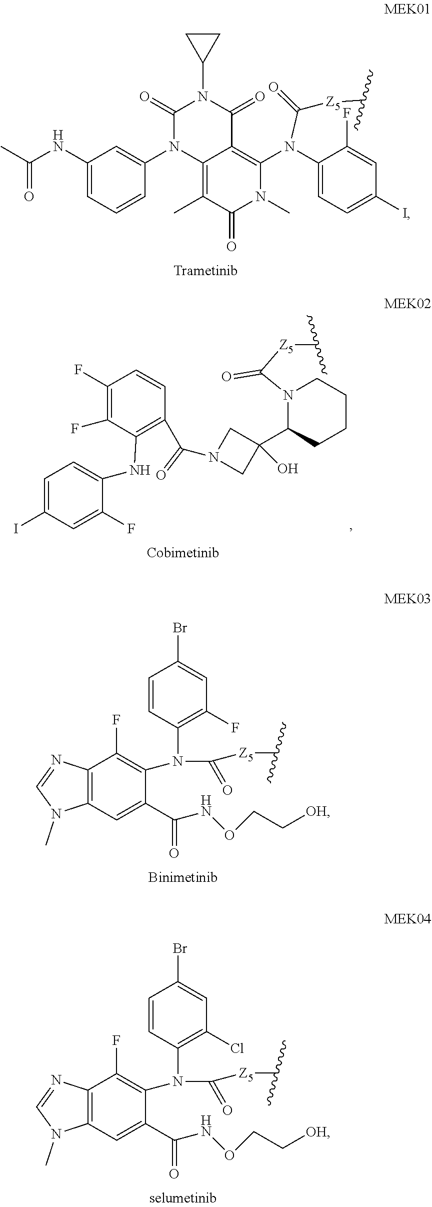

wherein Z₅ is selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁;

A proteinase inhibitor that are used as a payload is preferably selected from: Carfilzomib, Clindamycin, Retapamulin, Indibulin, as shown in the following structures:

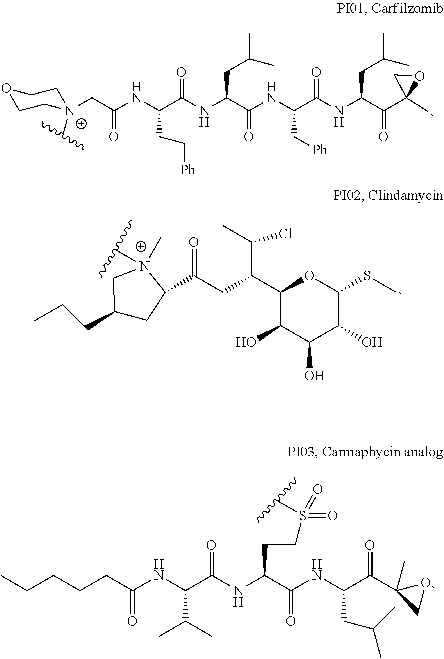

An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA’), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves; An immunotoxin herein is preferably conjugated via the process of the application through an amino acid having free amino, thiol or carboxyl acid group; and more preferably through N-terminal amino acid.

In addition, a certain cell receptor agonist, a cell stimulating molecule or intracellular signalling molecule can be as a chemotherapeutic/function compound conjugated to BCMA antibody of the invention.

A cell-binding ligand or receptor agonist selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinizing hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gy-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gy-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and NodlikeReceptors' (NLRs) ligands; Calcitonin receptor agonists; integrin receptors' and their receptor subtypes' (selected from the group consisting of αvβ₁, αvβ₃, αvβ₅, αvβ₆, α₆β₄, α₇β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Anticalin (a derivative of Lipocalins); Adnectins (10th FN3 (Fibronectin)); Designed Ankyrin Repeat Proteins (DARPins); Avimers; EGF receptors, or VEGF receptors' agonists;

A cell-binding molecule/ligand or a cell receptor agonist selected from the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an αᵥ integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) which are shown in the following structures:

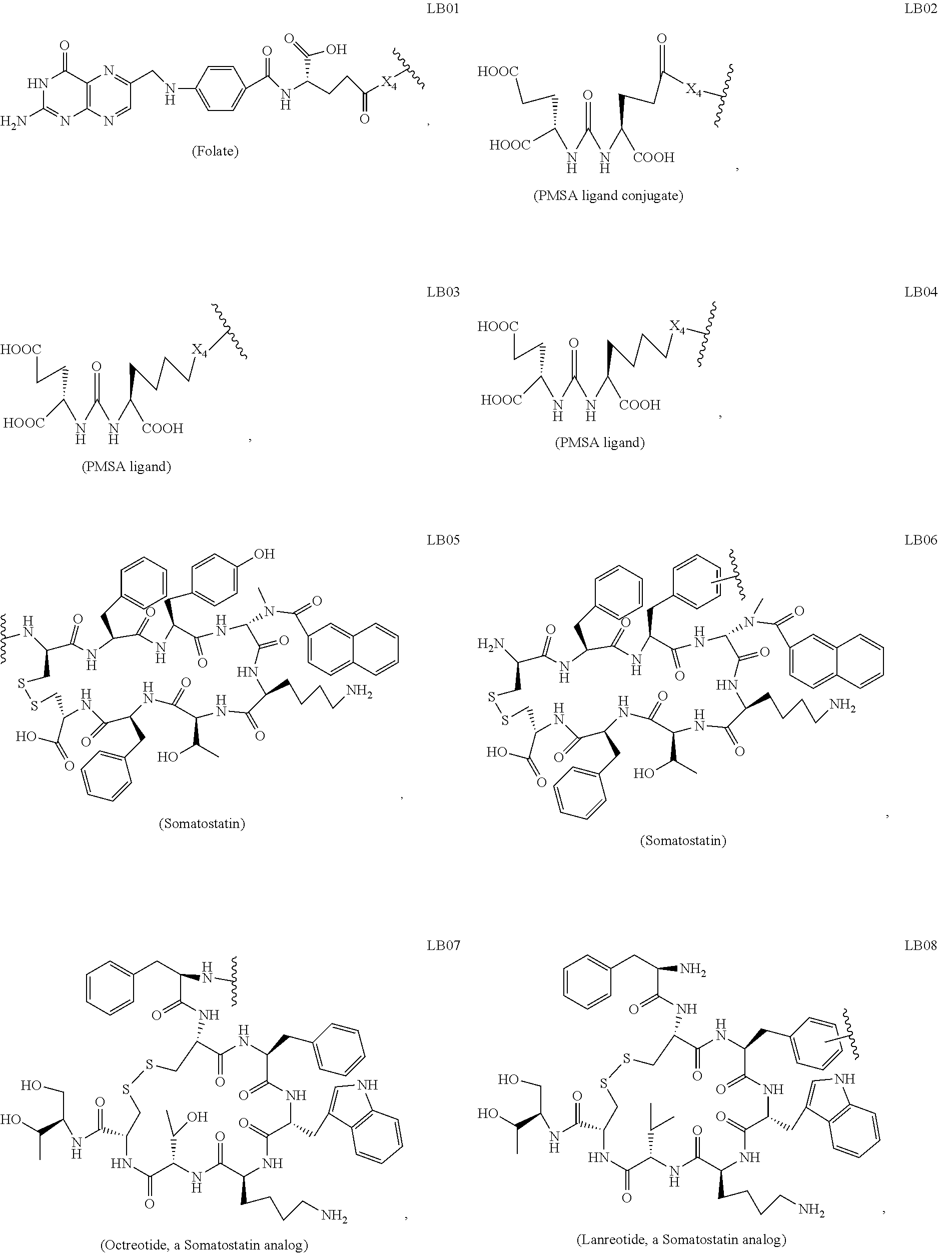

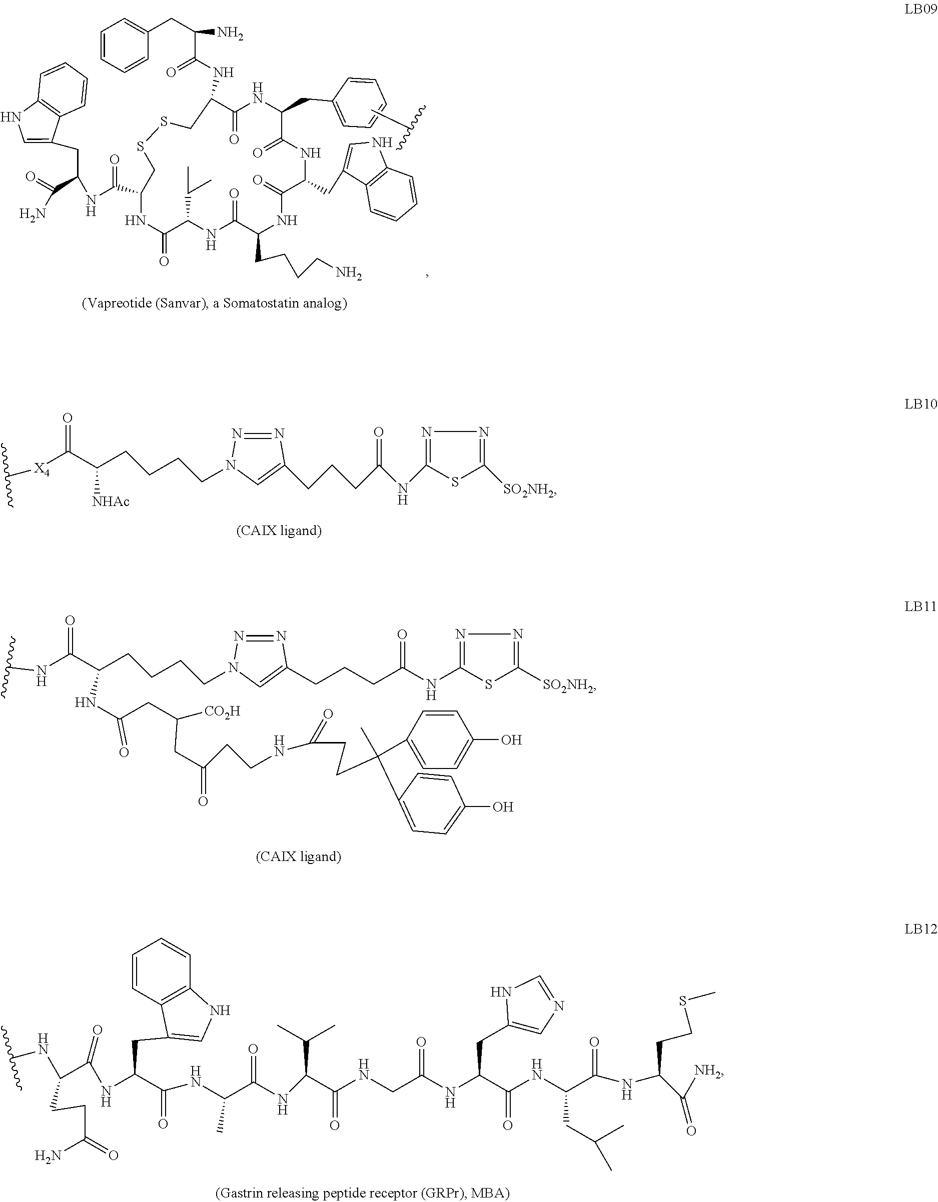

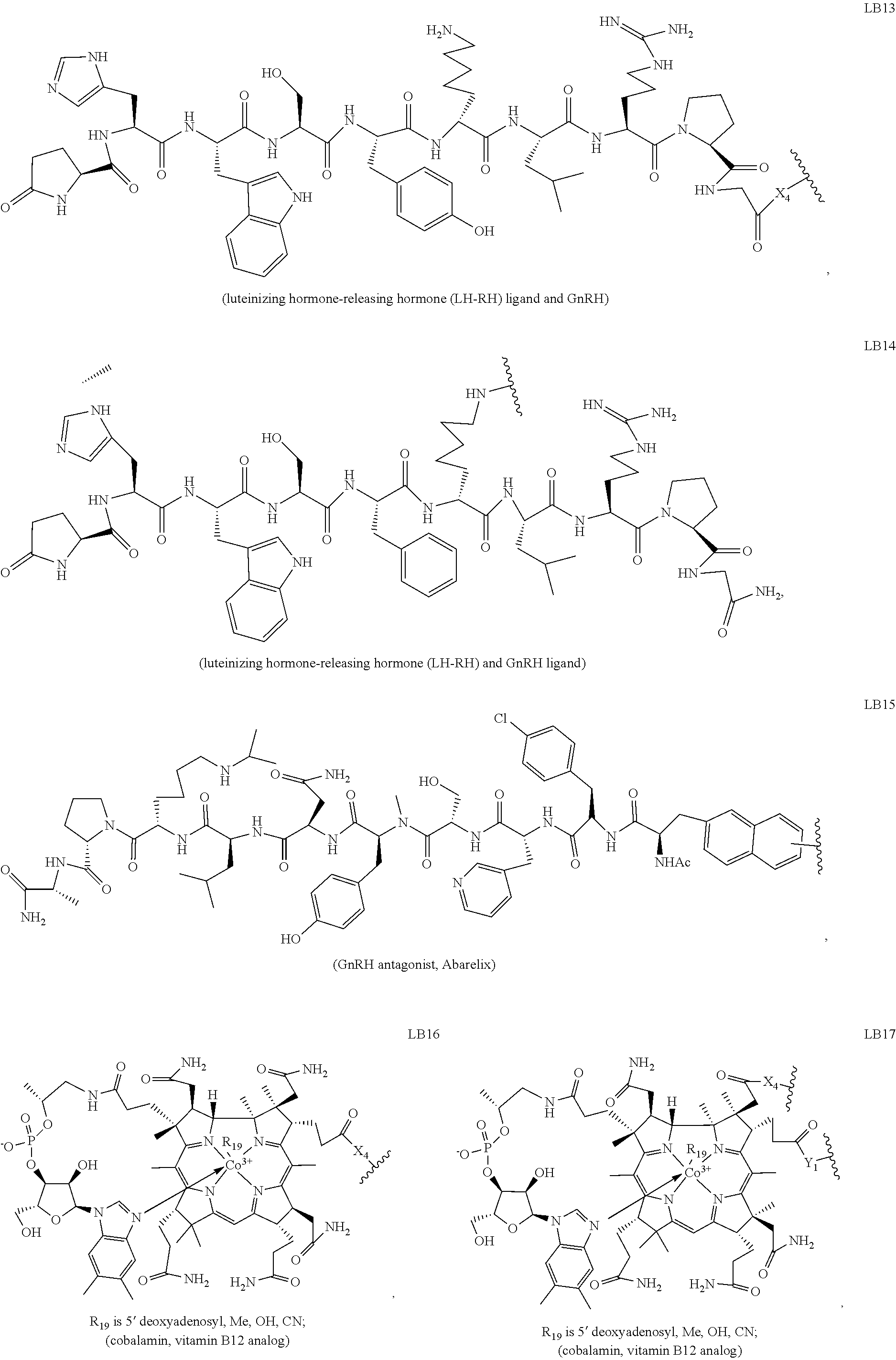

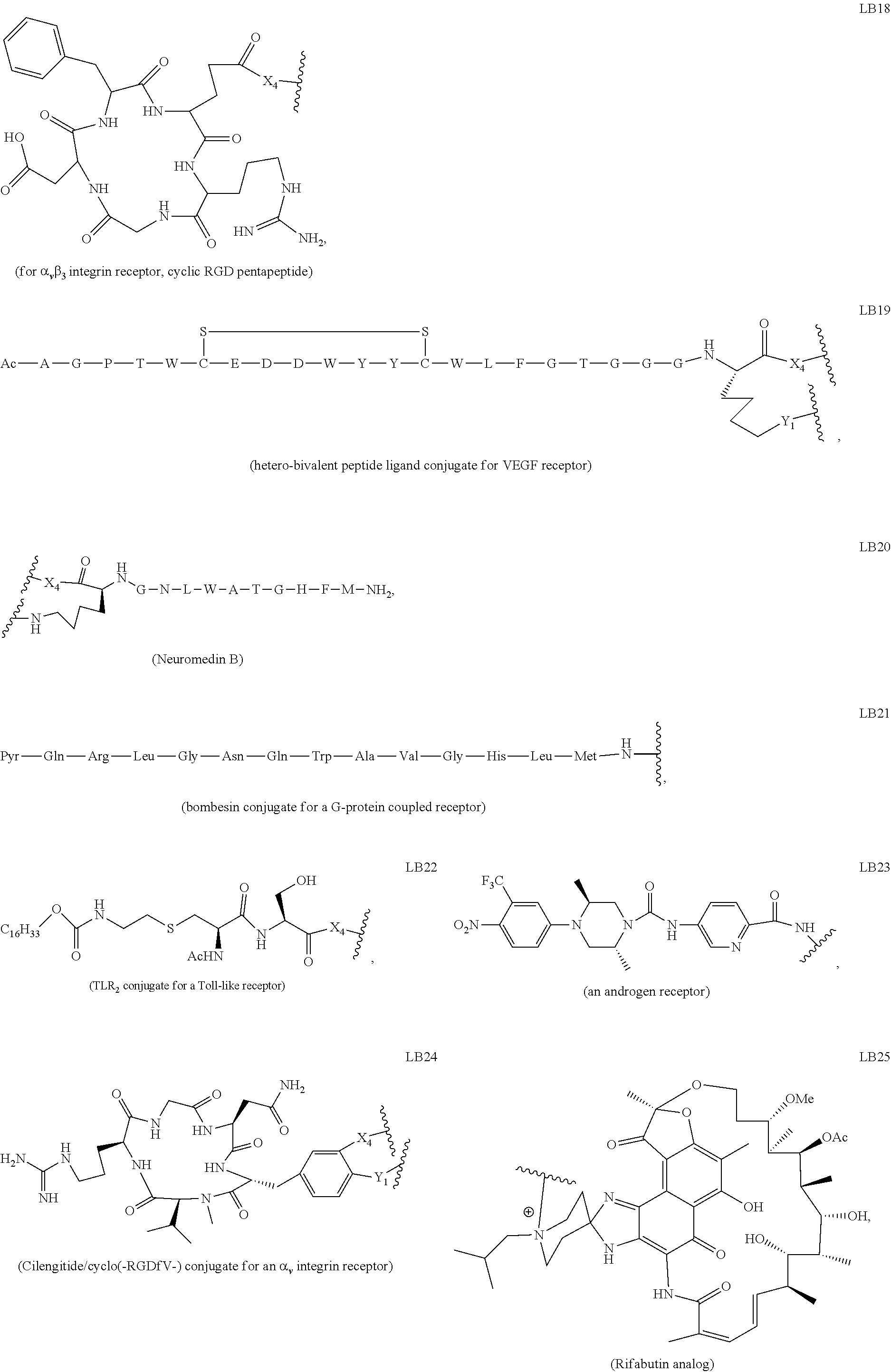

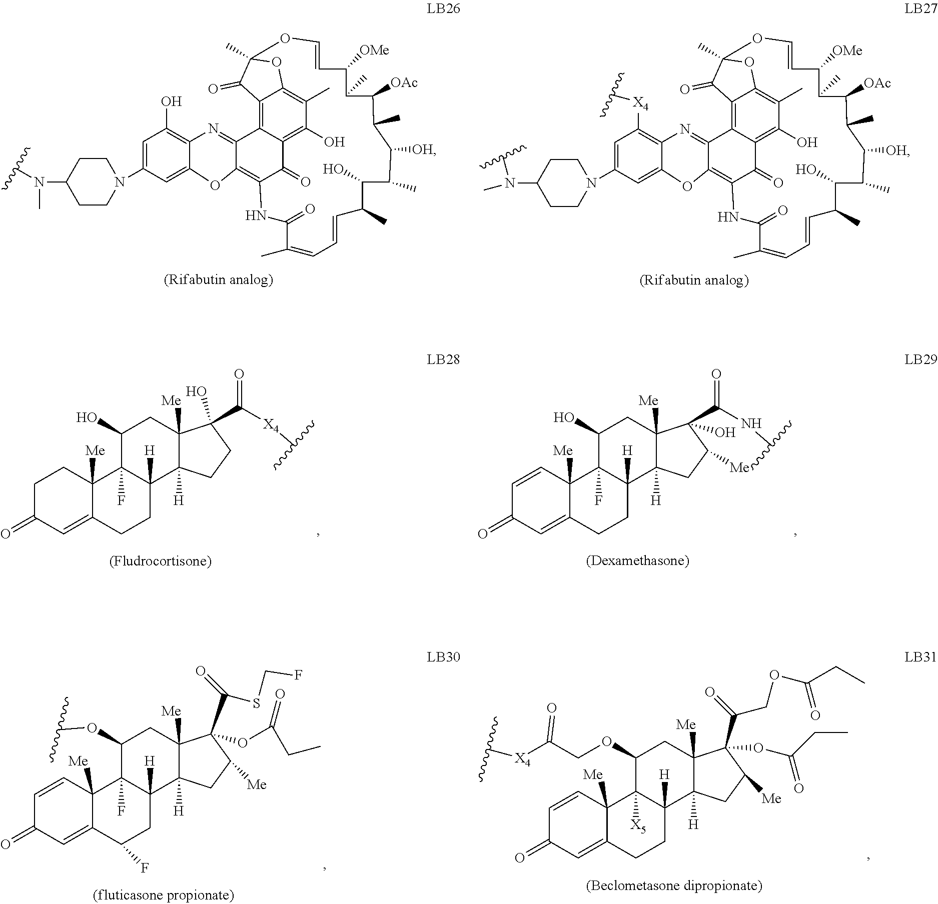

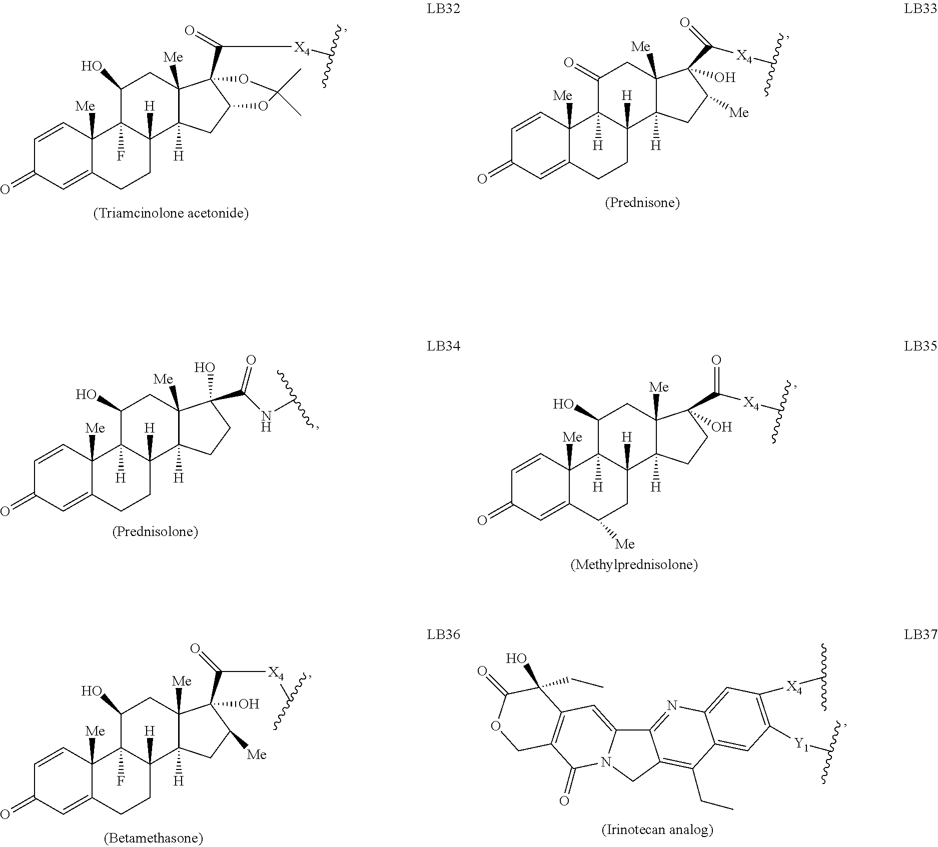

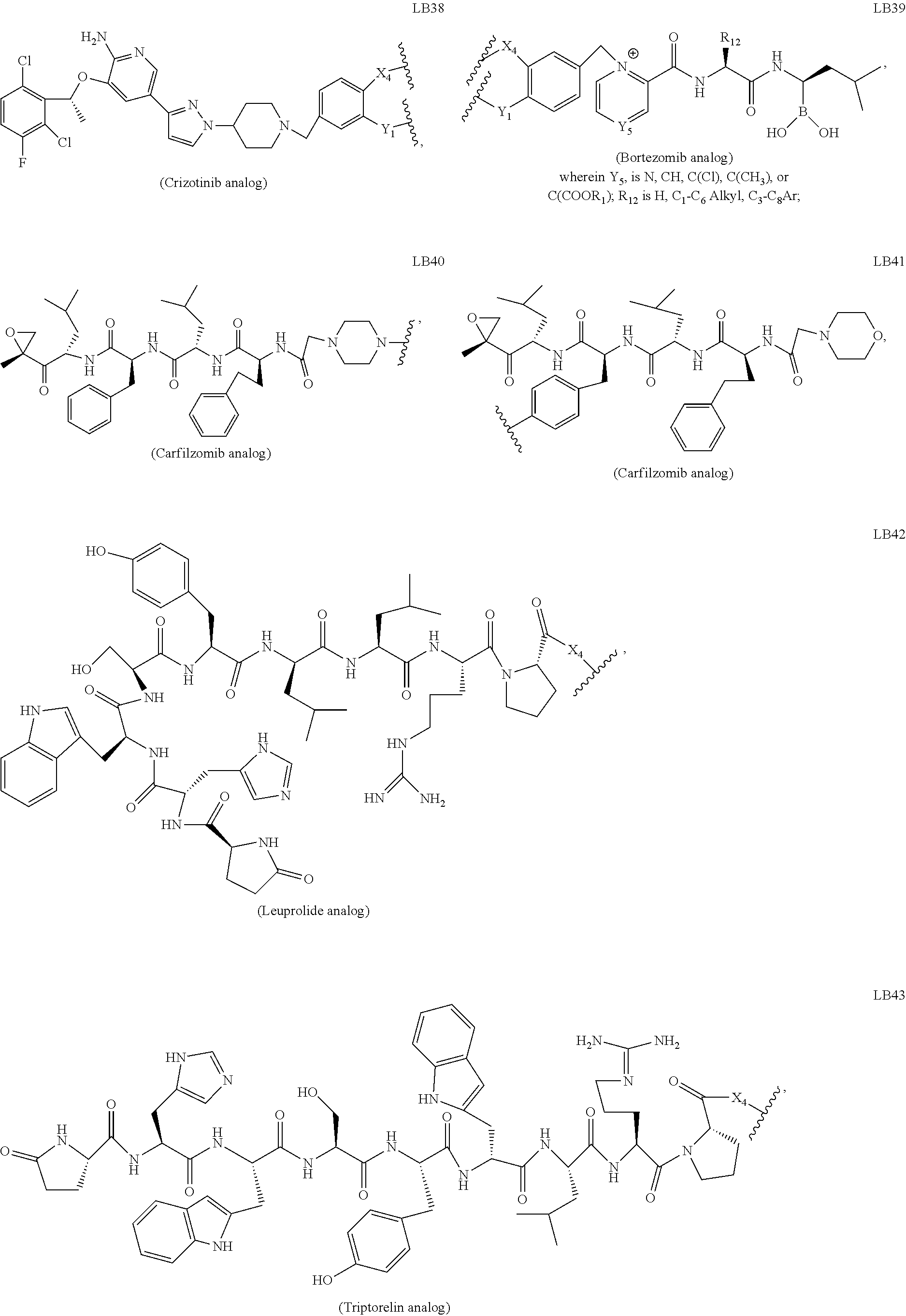

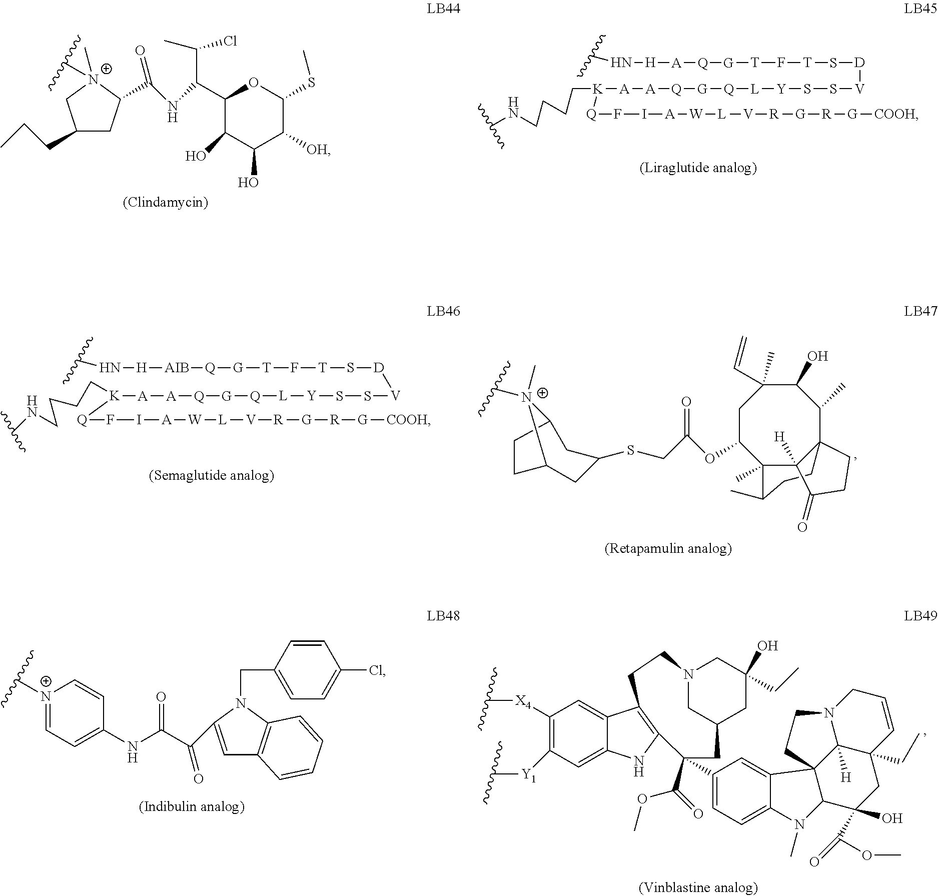

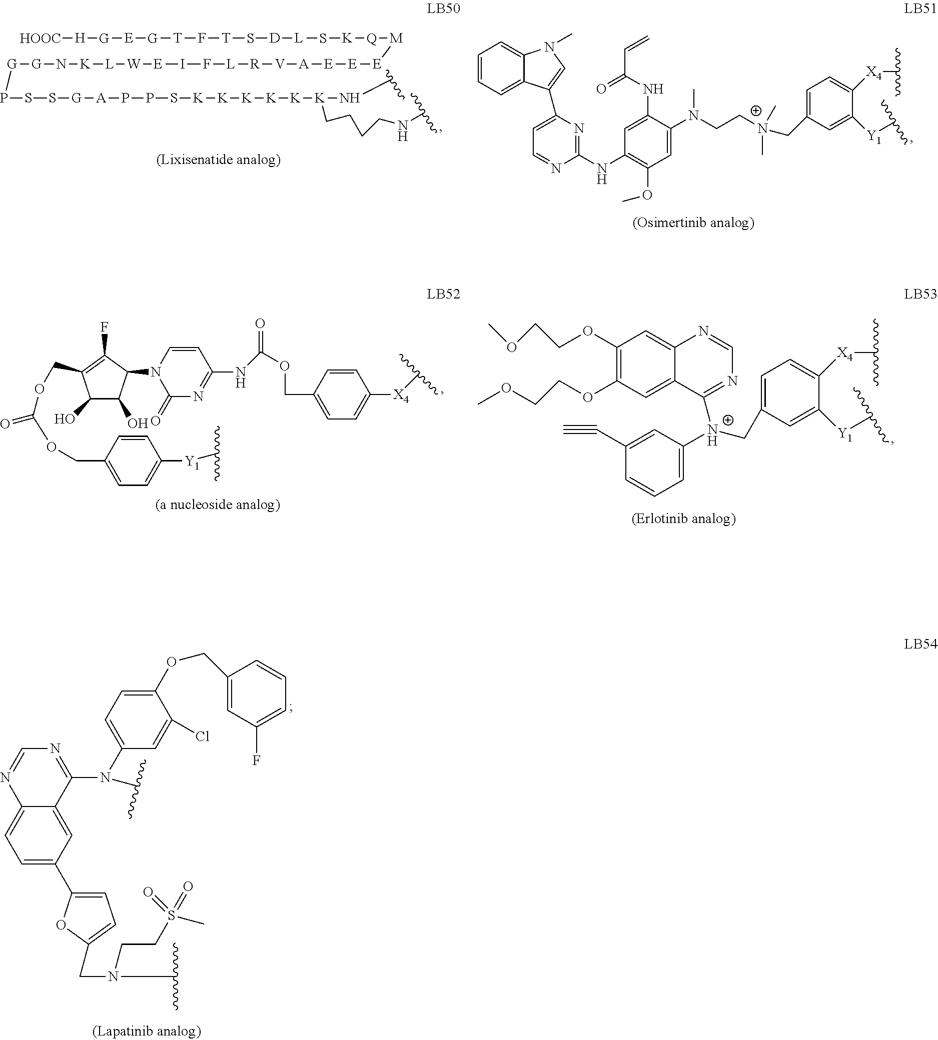

Wherein X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁.

In certain embodiments, one, two or more DNA, RNA, mRNA, small interfering RNA (siRNA), micro RNA (miRNA), and PIWI interacting RNAs (piRNA) can be as a chemotherapeutic/function compound conjugated to BCMA antibody of the invention:

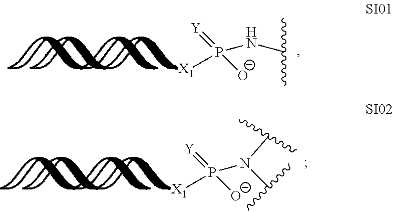

wherein “ [symbol] ” is the site to link the side chain linker of the present patent; [symbol] is single or double strands of DNA, RNA, mRNA, siRNA, miRNA, or piRNA; X₁ and Y are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁.

The linker L₁ and L₂ are, the same or different, independently selected from O, NH, S, S—S, NHNH, N(R₃), N(R₃)N(R_{3′}), C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; C₂-C₈ (2-8 carbon atoms) of esters, ether, or amide; 1˜8 natural or unnatural amino acids described in the definition; polyethyleneoxy unit of formula (OCH₂CH₂)ₚ, (OCH₂CH(CH₃))ₚ, (OCH₂CH₂)ₚOR₃, (OCH₂CH(CH₃))ₚOR₃, NH(CH₂CH₂O)ₚR₃, NH(CH₂CH(CH₃)O)ₚR₃, N[(CH₂CH₂—O)ₚR₃][(CH₂CH₂O)ₚR₃′], (OCH₂CH₂)ₚCOOR₃, or CH₂CH₂(OCH₂CH₂)ₚCOOR₃, wherein p and p′ are independently an integer selected from 0 to about 1000, or combination thereof, wherein R₃ and R₃′ are independently H; C₁-C₈ of alkyl; C₂-C₈ of heteroalkyl, alkylcycloalkyl, heterocycloalkyl; C₃-C₈ of aryl, Ar-alkyl, heterocyclic, cycloalkyl, heteroalkylcycloalkyl, alkylcarbonyl, heteroaryl;

L₁ or L₂ may contain a self-immolative or a non-self-immolative component, peptidyl units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals.

Preferably, the self-immolative linker component has one of the following structures:

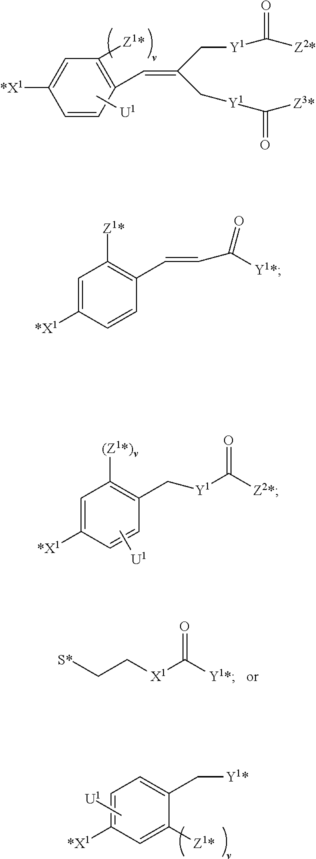

wherein the (*) atom is the point of attachment of additional spacer or releasable linker units, or the cytotoxic agent, and/or the antibody; X¹, Y¹, Z² and Z³ are independently NH, O, or S; Z¹ is independently H, NH, O or S; v is 0 or 1; U¹ is independently H, OH, C₁˜C₆ alkyl, (OCH₂CH₂)ₙF, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′) wherein R₅ and R₅′ are as defined above; preferably R₅ and R₅′ are independently selected from H, C₁˜C₈ alkyl; C₂˜C₈ alkenyl, alkynyl, heteroalkyl; C₃˜C₈ aryl, heterocyclic, carbocyclic, cycloalkyl, heterocycloalkyl, heteroaralkyl, alkylcarbonyl; or pharmaceutical cation salts.

The non-self-immolative linker component is one of the following structures:

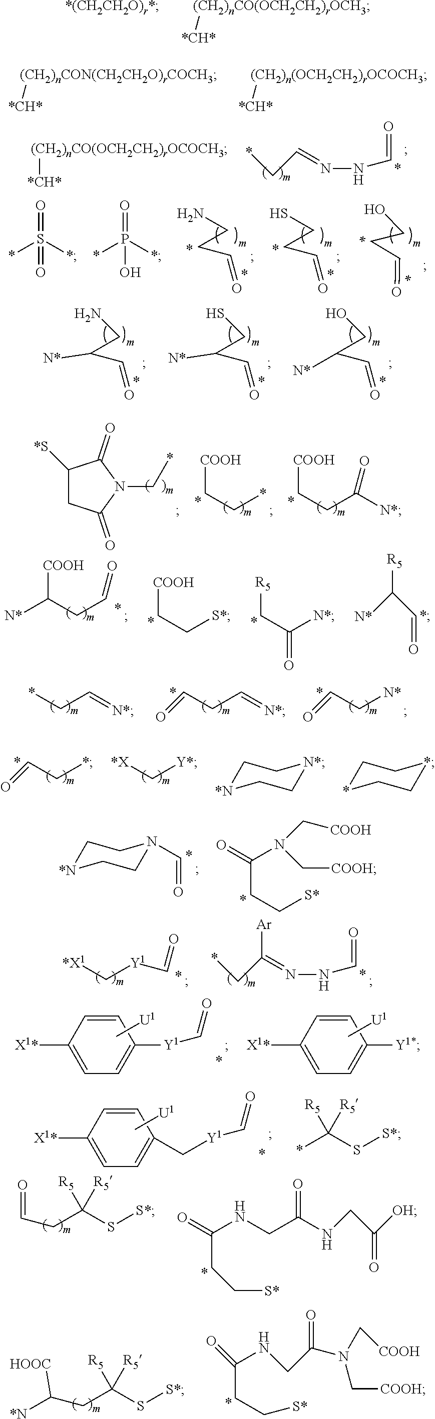

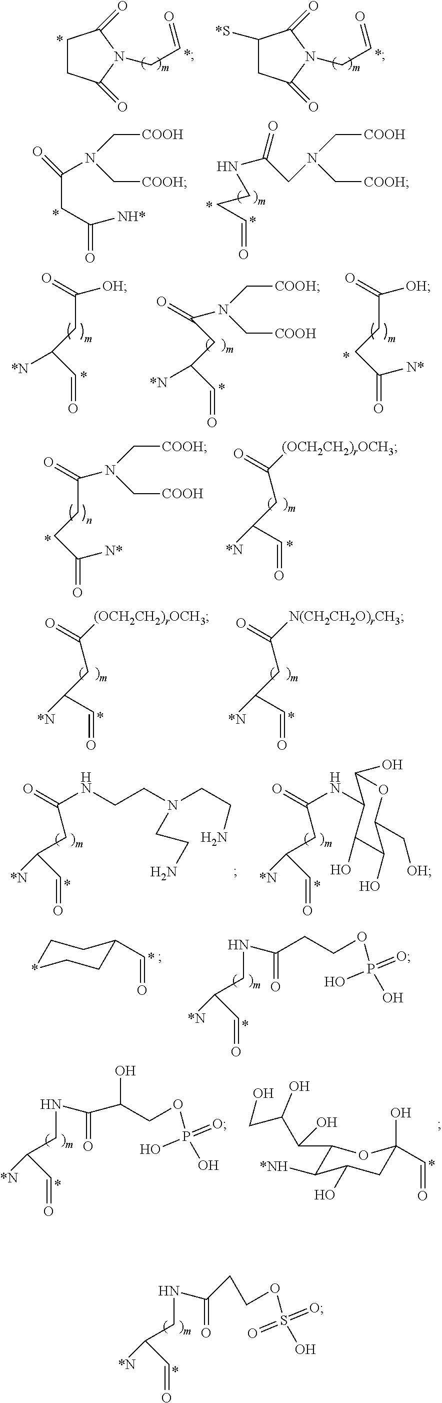

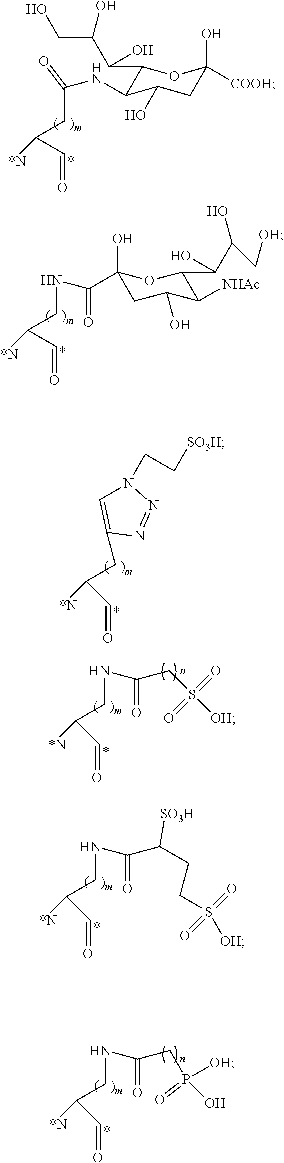

Wherein the (*) atom is the point of attachment of additional spacer R₁ or releasable linkers, the cytotoxic agents, and/or the binding molecules; X¹, Y¹, U¹, R₁, R₅, R₅' are defined as above; r is 0˜100; m and n are 0˜6 independently.

More preferably, L₁ or L₂ may be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites.

Further preferably, L₁ or L₂ may be a releasable linker. The term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with an intracellular thiol, such as a millimolar range of abundant of glutathione inside the malignant cells.

Examples of the releasable linkers (L, L₁ or L₂) include, but not limited:
—(CR₅R₆)ₘ(Aa)r(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)ᵣ(CR₅R₆)ₘ(C R₇R₈)ₙ(OCH₂CH₂)ₜ—, —(CR₅R₆)ₘ(CR₇R₈)ₙ(OCH₂CH₂)ᵣ(Aa)ₜ-, -(CR₅R₆)ₘ(CR₇═CR₈)(CR₉R₁₀)ₙ(Aa)ₜ-(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ—(OCH₂CH₂)ᵣ—, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ—(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ—(OCH₂CH₂)ᵣ—, —(CR₅R₆)ₘ(CO)(Aa)ₜ(CR₉R₁₀)ₙ—(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆)ₘthiazolyl-CO- (Aa)ₜ(CCR₇R₈)ₙ—, —(CR₅R₆)ₜ-thienyl-CO-(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ(CR₇R₈)ₙ—, —(CR₅R₆)ₜ-N-methyl-piperazin-CO(Aa)ₜ-(CR₇R₈)ₙ—, —(CR₅R)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(C R⁵R⁶)ₘ-morpholino-(Aa)ₜ-, —(CR₅R₆)ₘ-piperazino-(Aa)ₜ-, —(CR₅R₆)ₘN-methylpiperazino-(Aa)ₜ-, —K(CR₅R₆)ₘ(Aa)r(CR₇R₈)ₙ(OCH₂CH₂)ₜ—, —K(CR₅R₆)ₘ(CR₇R₈)ₙ-(Aa)ᵣ(OCH₂CH₂)ₜ—, —K(Aa)ᵣ(CR₅R₆)ₘ—(CR₇R₈)ₙ(OCH₂CH₂)ₜ—, —K(CR₅R₆)ₘ(CR₇R₈)ₙ—(OCH₂CH₂)ᵣ(Aa)ₜ-, —K(CR₅R₆)ₘ(CR₇═CR₈)(CR₉R₁₀)ₙ-(Aa)ₜ(OCH₂CH₂)ᵣ—, —K(CR₅R₆)ₘ—(NR₁₁CO)(Aa)ₜ(CR₉R₁₀)ₙ(OCH₂CH₂)ᵣ—, —K(CR₅R₆)ₘ(Aa)ₜ(NR₁₁CO)—(CR₉R₁₀)ₙ(OCH₂—CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ-(CR₉R₁₀)ₙ—(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)-(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R⁶)ₘ(OCNR₇)-(Aa)ₜ(CR₉R₁₀)ₙ(OCH₂CH₂)ᵣ—, —K(CR₅R₆)ₘ(CO)(Aa)ₜ(CR₉R₁₀)ₙ—(OCH₂CH₂)ᵣ—, —K(CR₅R⁶)ₘ- phenyl-CO-(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘthiazolyl-CO(Aa)ₜ-(CR₇R₈)ₙ—, —K(CR₅R₆)ₜ-thienyl-CO(CR₇R₈)ₙ—, —K(CR₅R₆)ₜimidazolyl-CO-(CR₇R₈)ₙ—, —K(CR₅R₆)ₜmorpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ-N-methylpiperazinCO(Aa)ₜ(CR₇R₈)ₙ—, —K(CR₅R)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘpiperazino-(Aa)ₜG, —K(CR₅R₆)ₘN-methylpiperazino-(Aa)ₜ-; wherein m, Aa, m, n, R₃, R₄, and R₅ are described above; t and r are 0-100 independently; R₆, R₇, and R₈ are independently chosen from H; halide; C₁˜C₈ of alkyl, aryl, alkenyl, alkynyl, ether, ester, amine oramide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B or C₃-C₆ heteroaromatic group.

Example structures of the components of the linker L₁ and L₂ may contain:

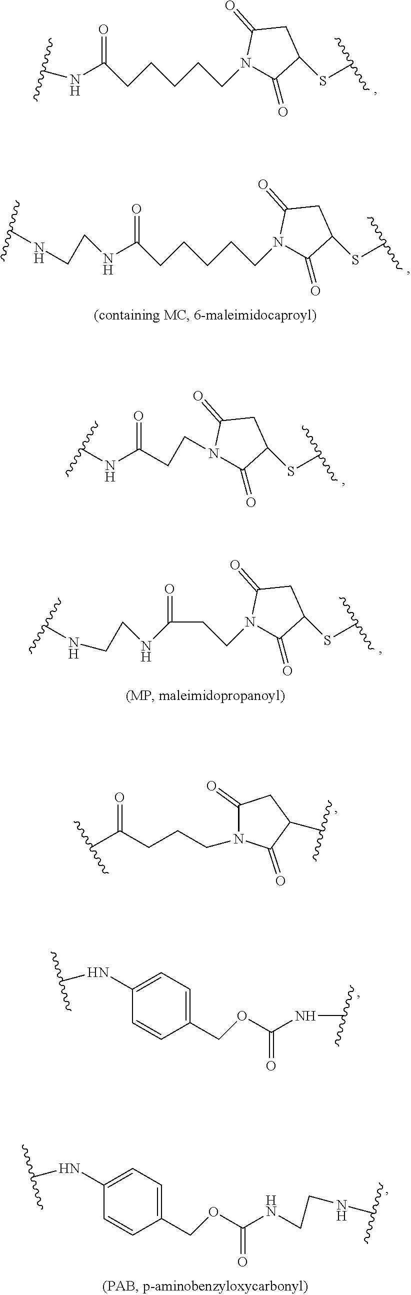

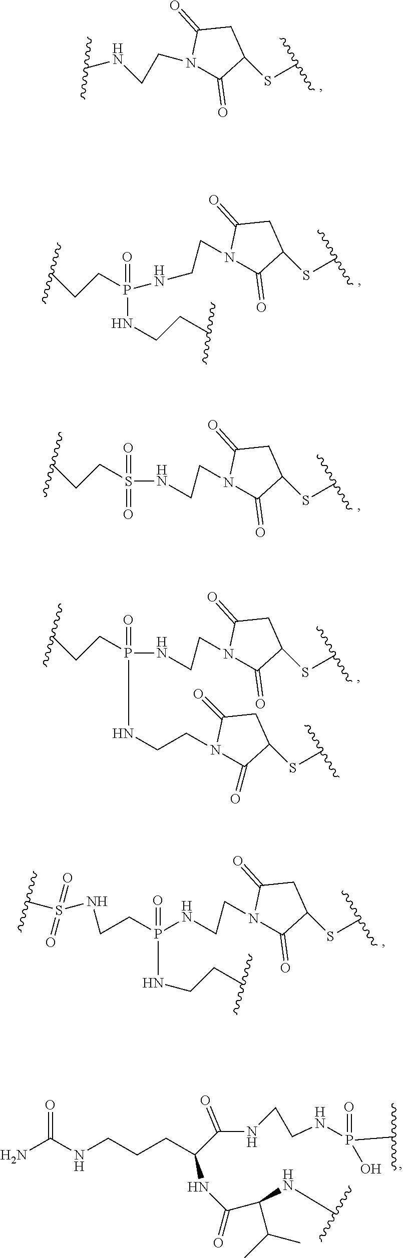 [SMILES: *NCCN1C(=O)CC(S*)C1=O ; *CCNP(=O)(CC*)NCCN1C(=O)CC(S*)C1=O ; *CCS(=O)(=O)NCCN1C(=O)CC(S*)C1=O ; *CCP(=O)(NCCN1C(=O)CC(S*)C1=O)NCCN1C(=O)CC(S*)C1=O ; *CCNP(=O)(CCNS(*)(=O)=O)NCCN1C(=O)CC(S*)C1=O ; *N[C@H](C(=O)N[C@@H](CCCNC(N)=O)C(=O)NCCNP(*)(=O)O)C(C)C]

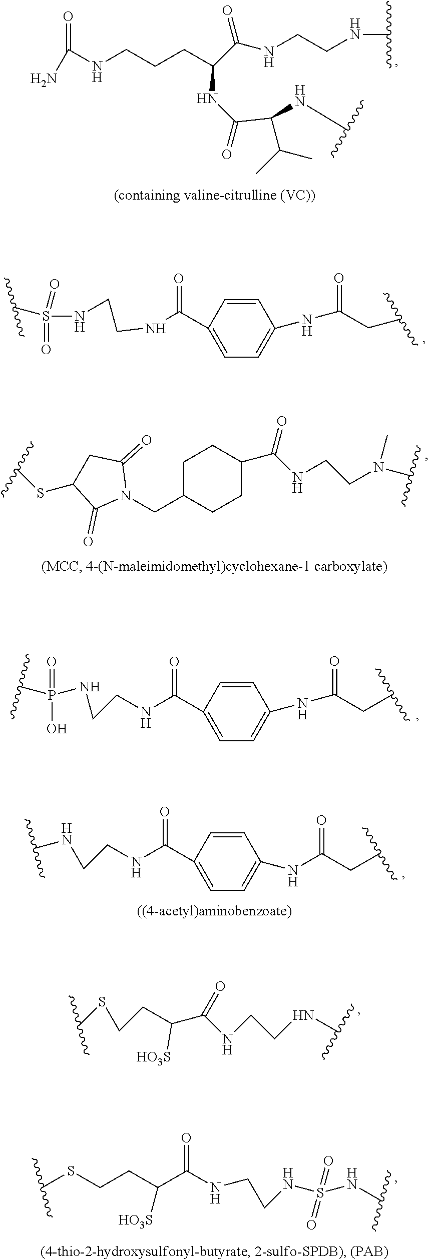

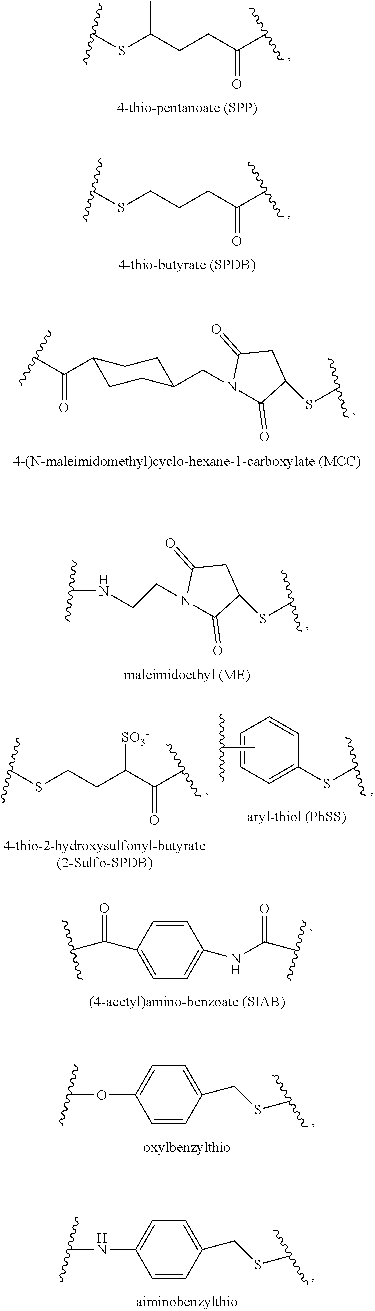

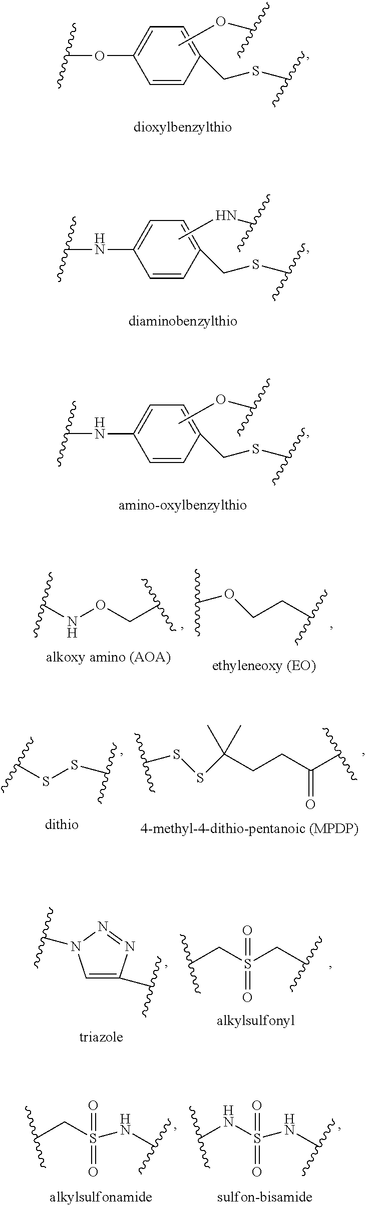

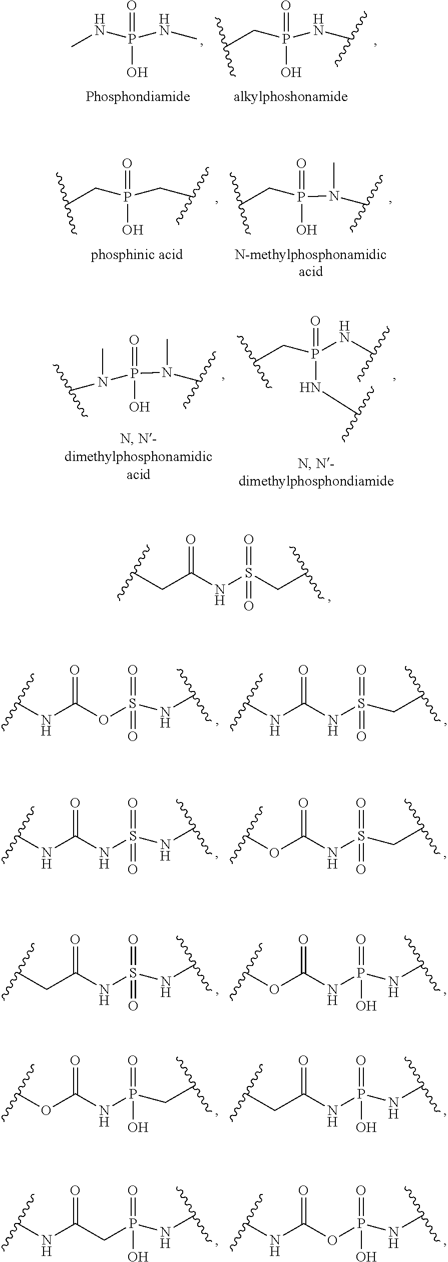

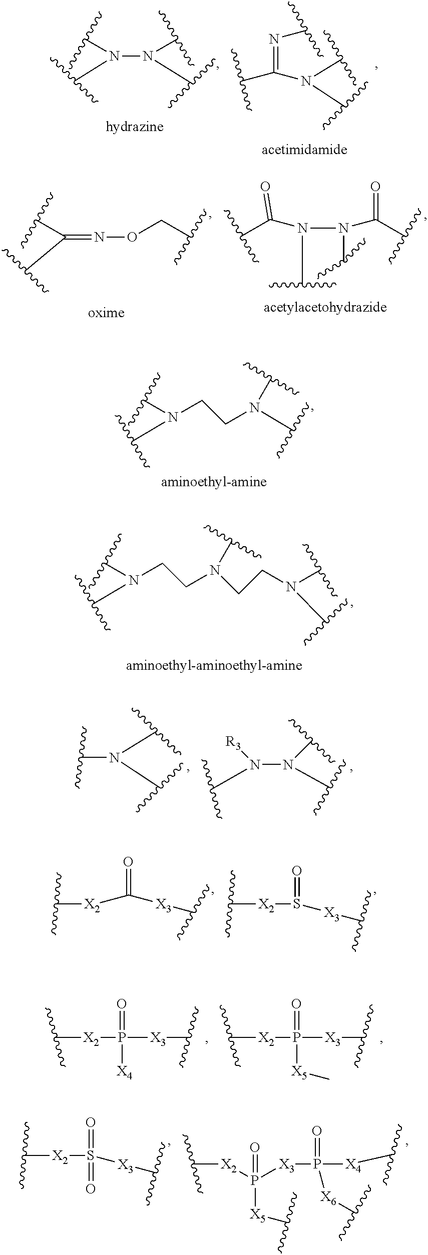

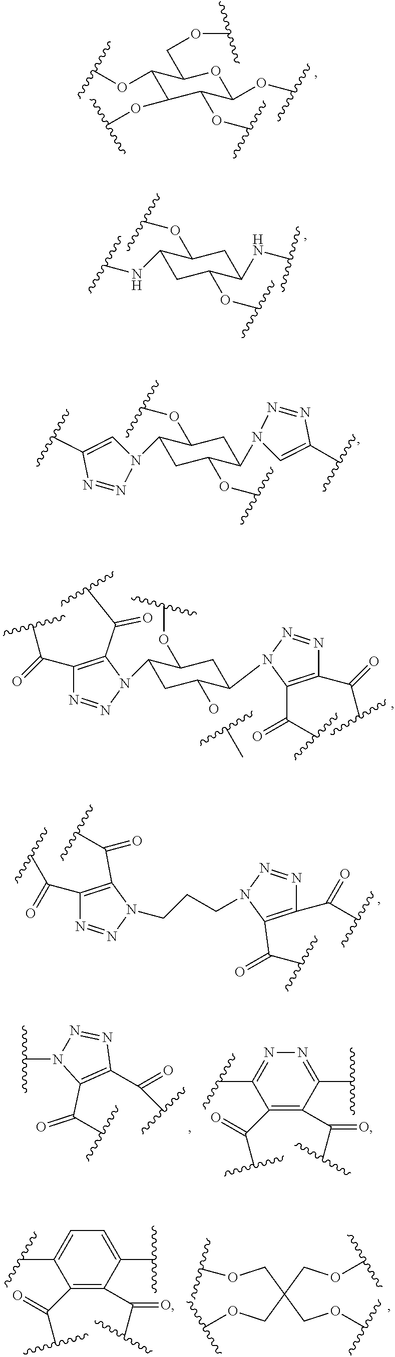

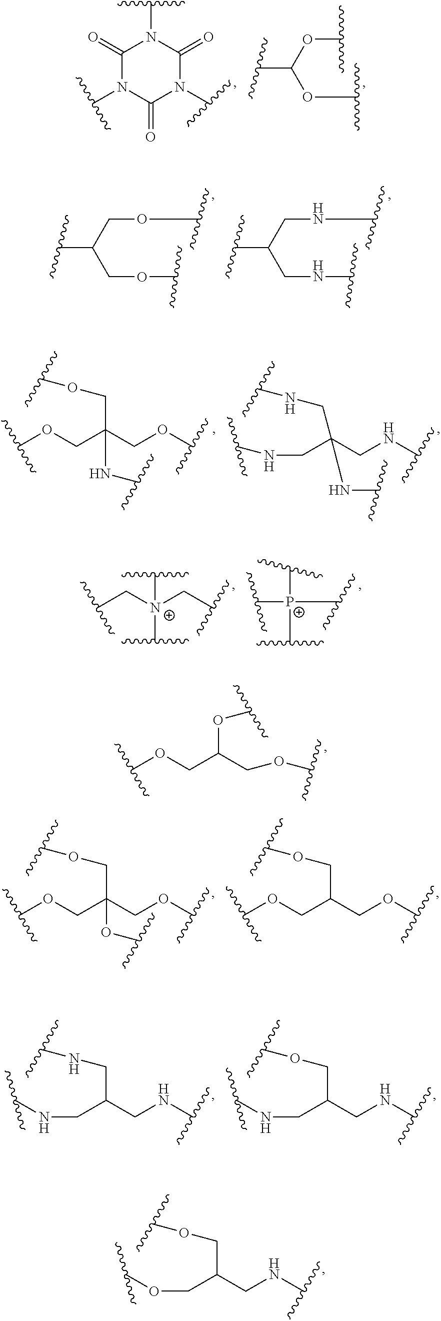

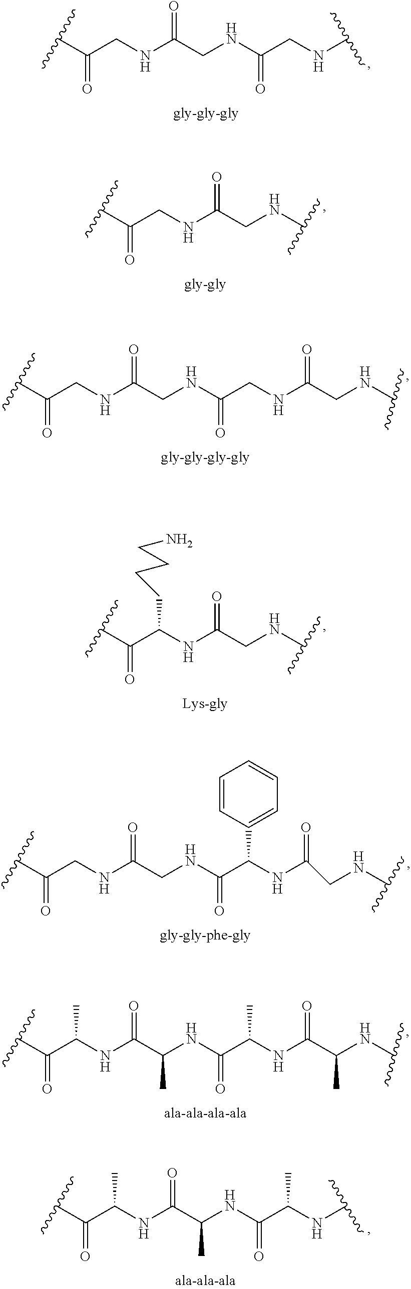

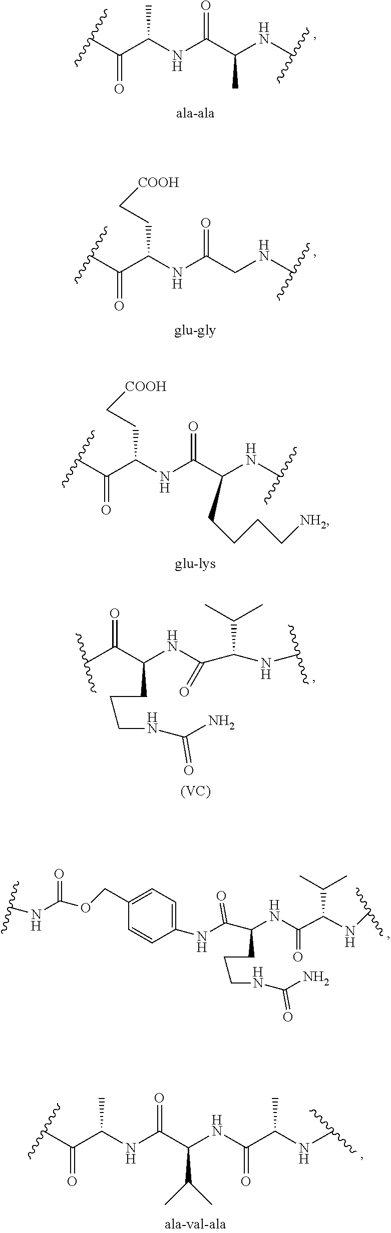

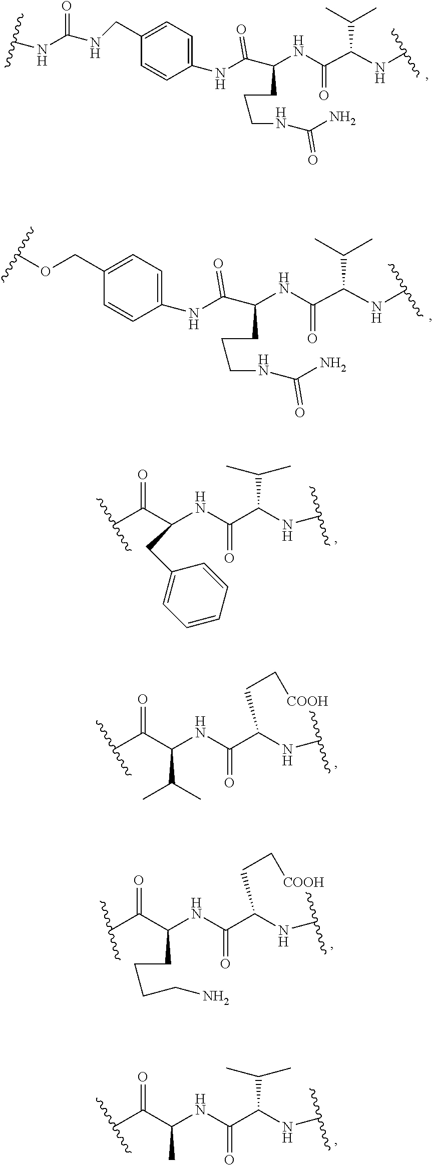 [SMILES: *NC(=O)NCc1ccc(NC(=O)[C@H](CCCNC(N)=O)NC(=O)[C@@H](N*)C(C)C)cc1 ; *N[C@H](C(=O)N[C@@H](CCCNC(N)=O)C(=O)Nc1ccc(CO*)cc1)C(C)C ; *N[C@H](C(=O)N[C@@H](Cc1ccccc1)C(*)=O)C(C)C ; *N[C@@H](CCC(=O)O)C(=O)N[C@H](C(*)=O)C(C)C ; *N[C@@H](CCC(=O)O)C(=O)N[C@@H](CCCCN)C(*)=O ; *N[C@H](C(=O)N[C@@H](C)C(*)=O)C(C)C]

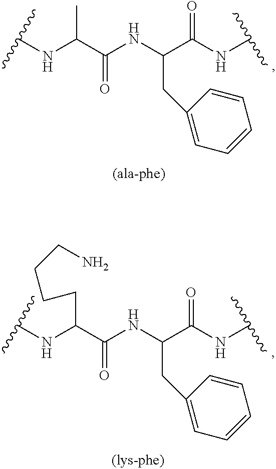 [SMILES: *NC(=O)C(Cc1ccccc1)NC(=O)C(C)N* ; *NC(=O)C(Cc1ccccc1)NC(=O)C(CCCCN)N*]

or a combination above thereof, wherein [symbol] is the site of linkage; X₂, X₃, X₄, X₅, or X₆, are independently selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1˜8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 100.

Preferably, the L₁ and L₂ are independently selected from:

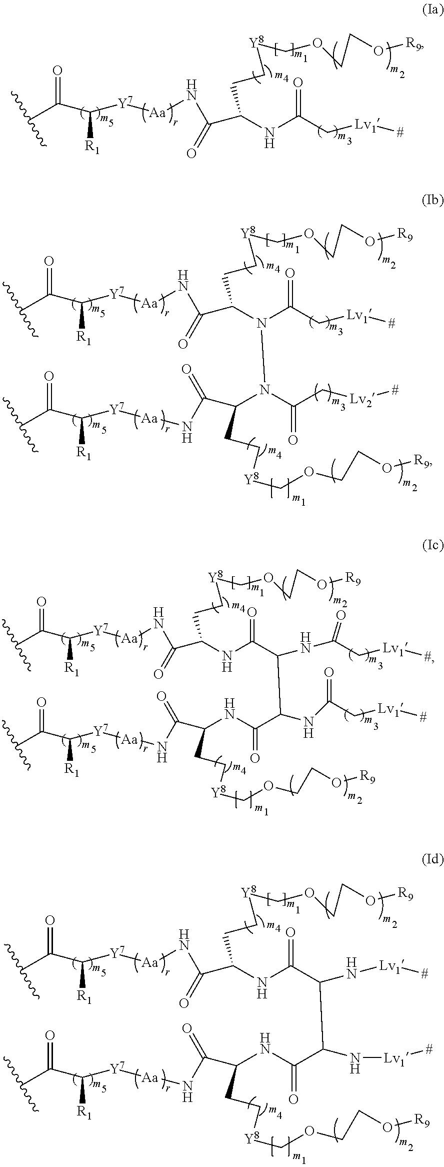

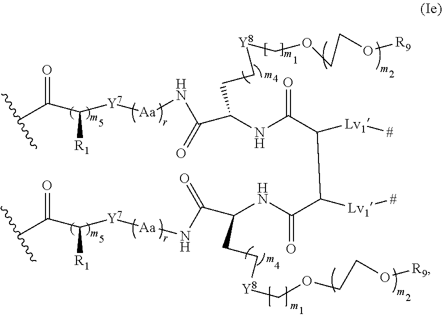

Wherein “ [symbol] ” is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; Aa is L- or D-natural or unnatural amino acids;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0-12; when r is not 0, (Aa)ᵣ is the same or different amino acids or peptide units;
m₁=1-18; m₂=1-100; m₃=1-8; m₄=0-8; m₅=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y⁸ is NHC(═O), NHS(O₂), NH(SO), NHS(O₂)NH, NHP(O)(OH)NH or C(O)NH;
R₉ is H, (O═)CR₁, (O═)CNHR₁, R₁COOH, R₁(COCH₂NH)ₘ₂H, R₁(Aa)ᵣ or R₁(COCH₂NCH₃)ₘ₂H, wherein R₁ is defined above;
Lv_{1′} is selected from:

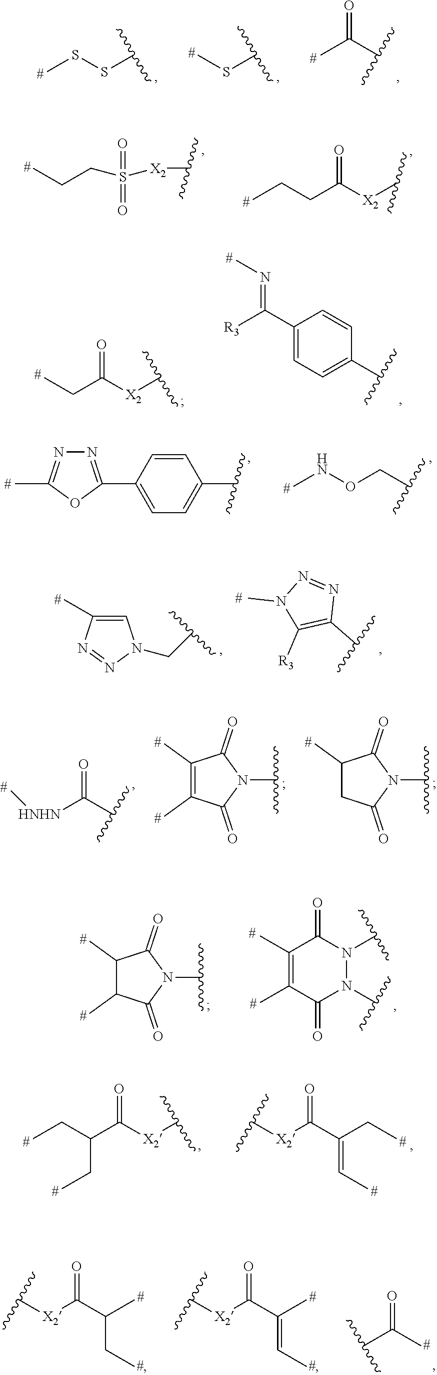

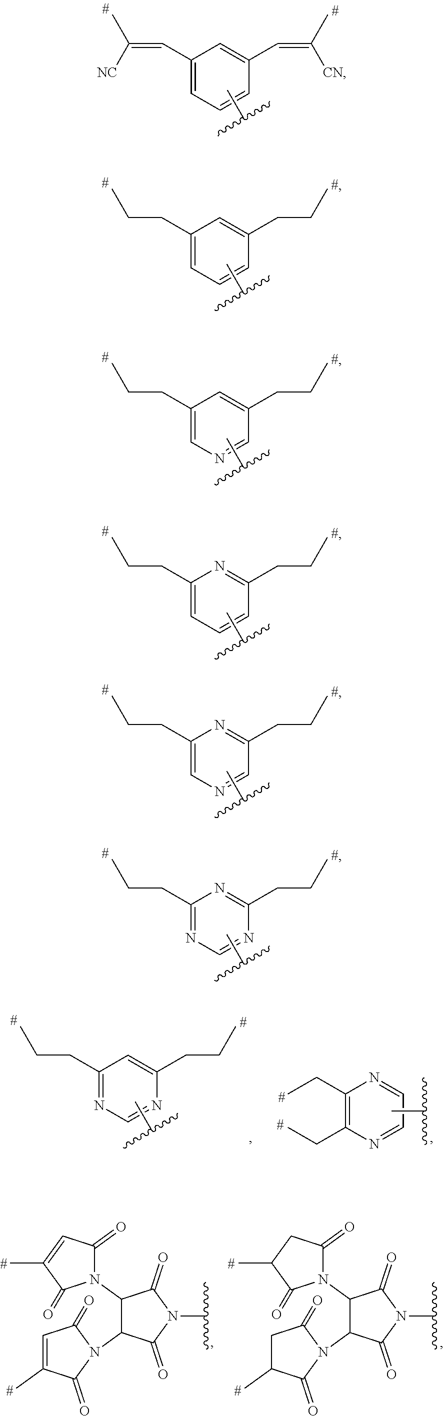

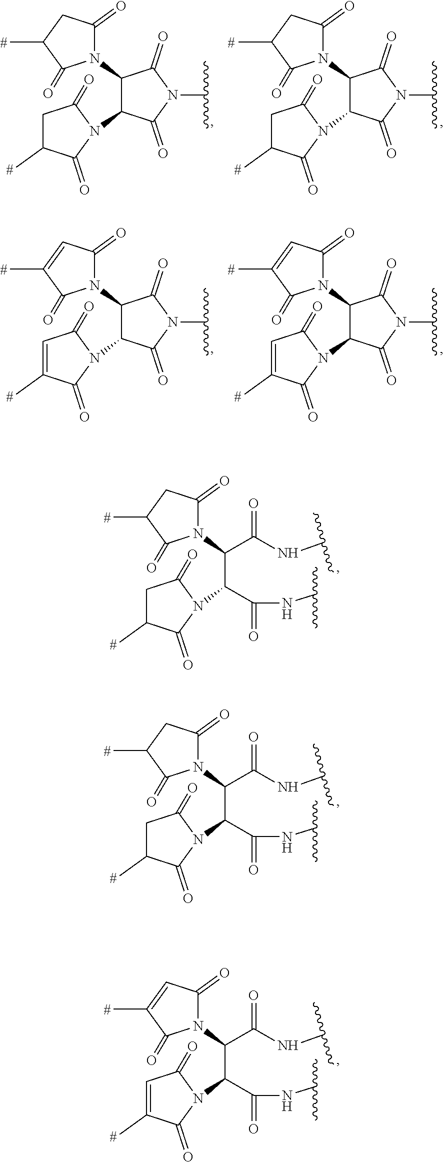

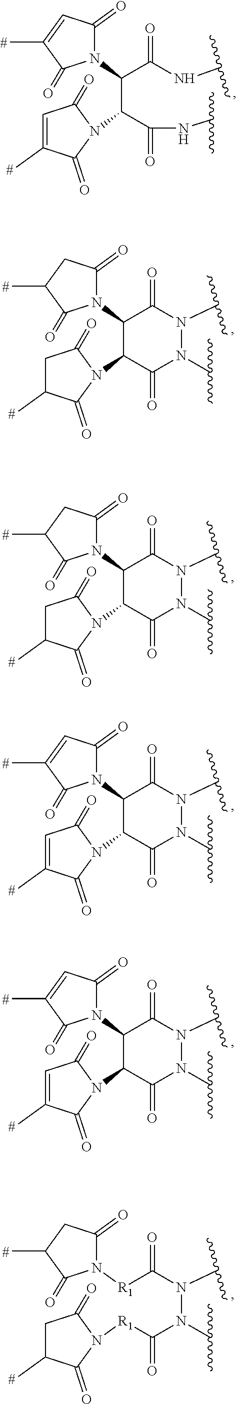

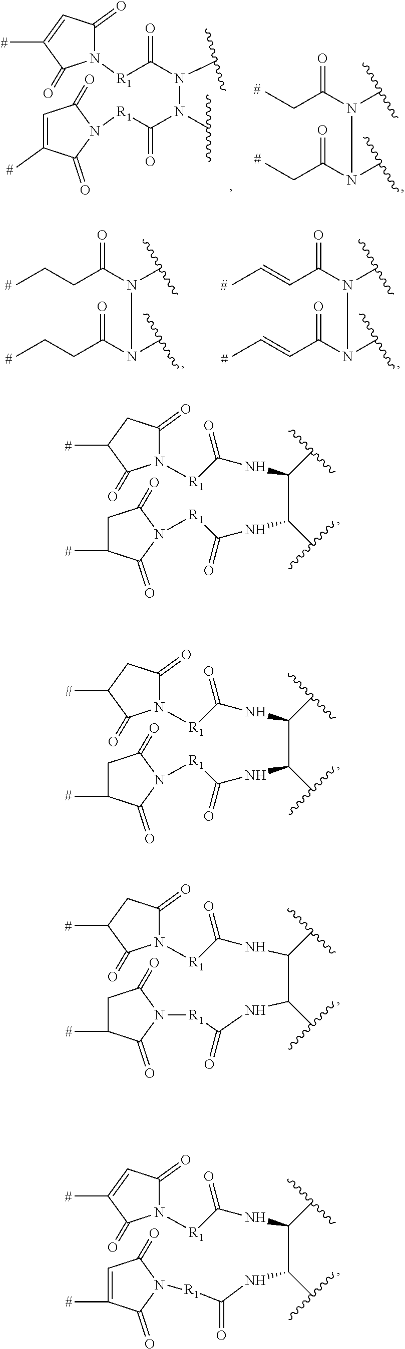

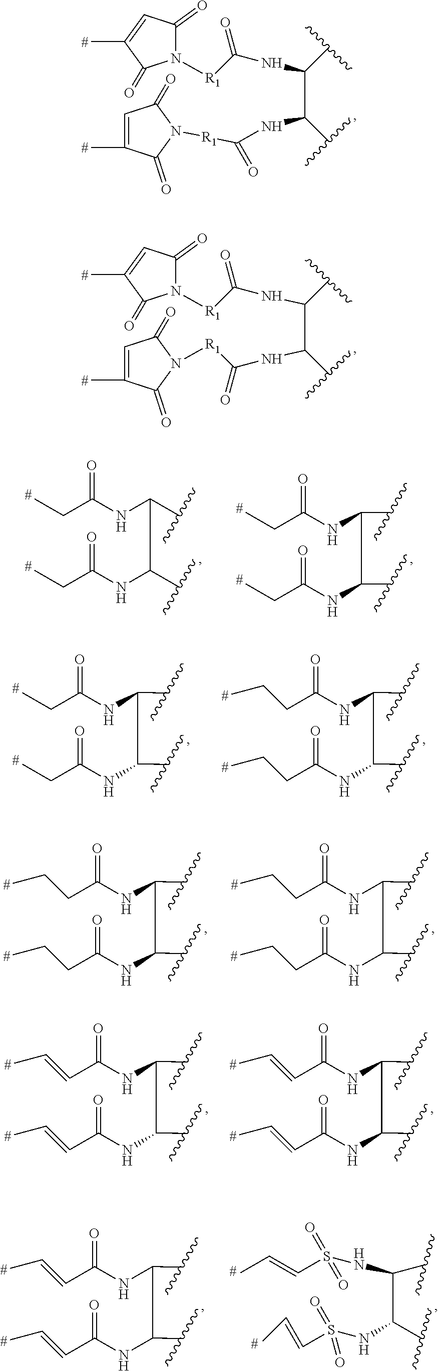

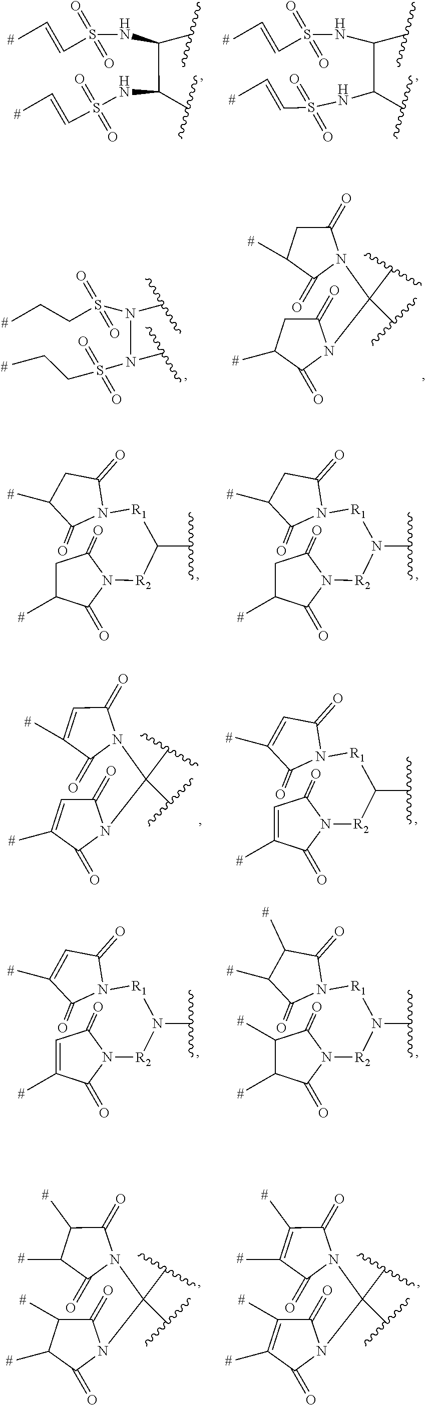

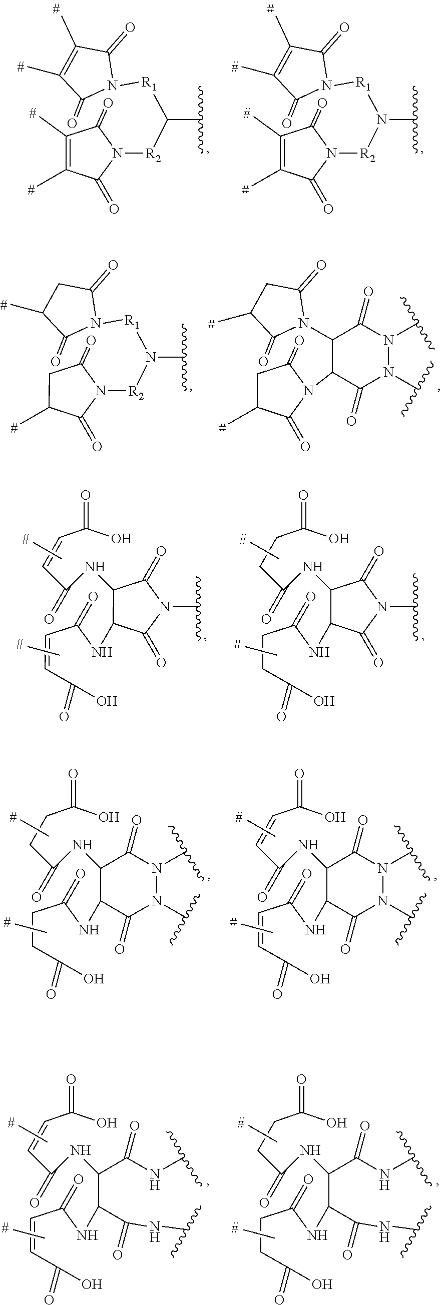

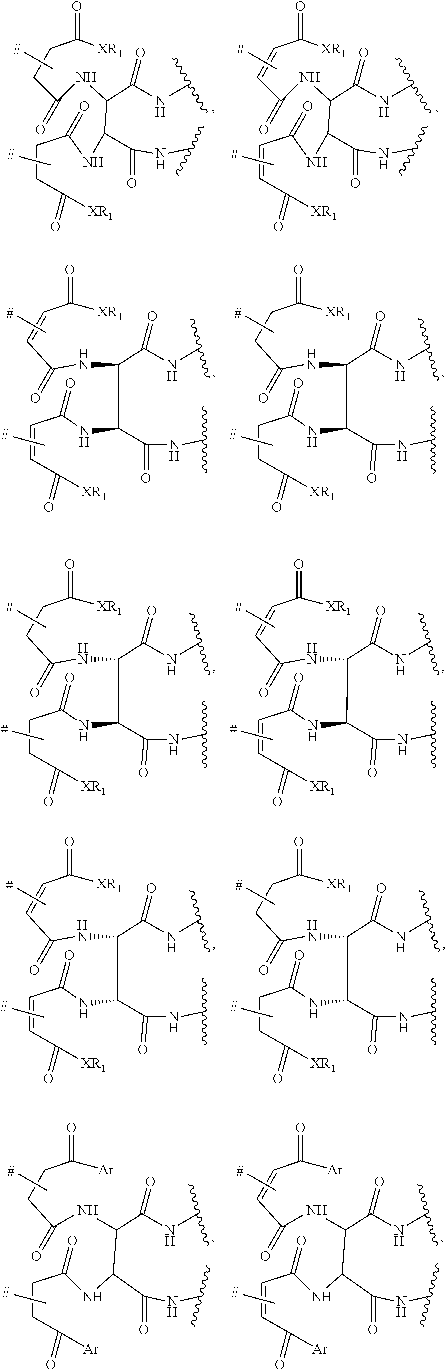

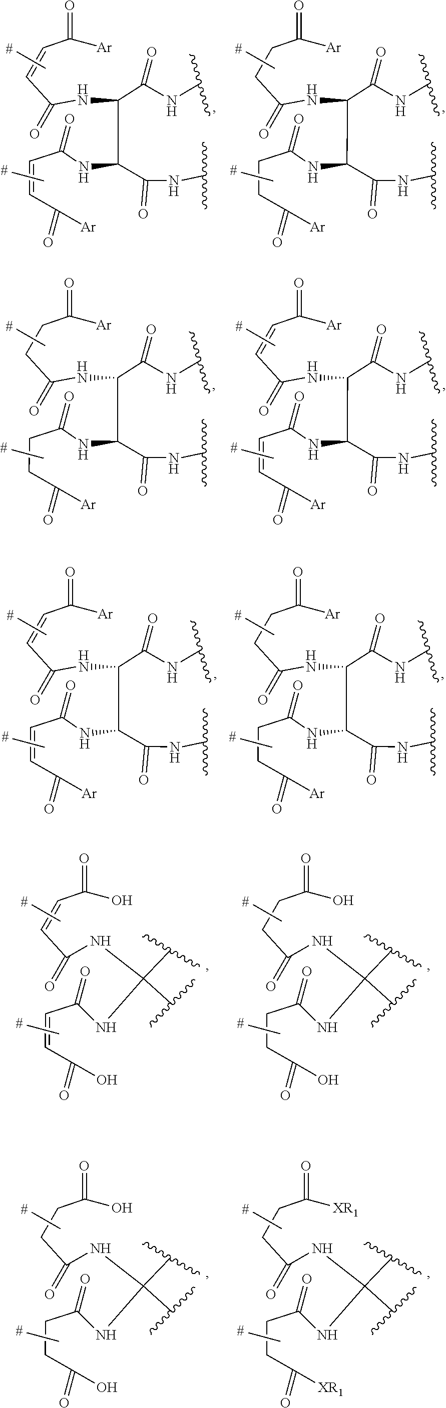

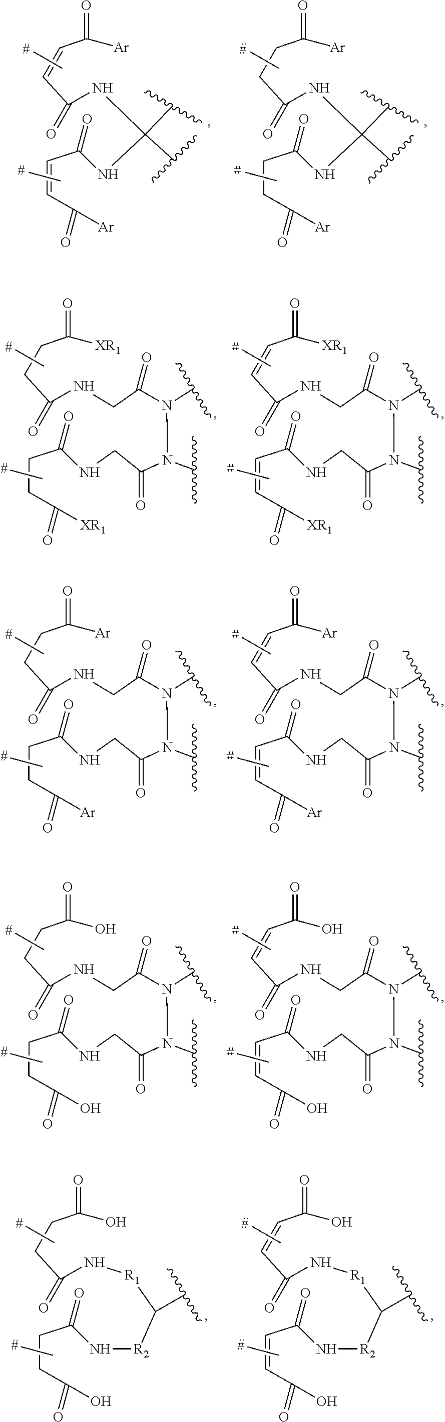

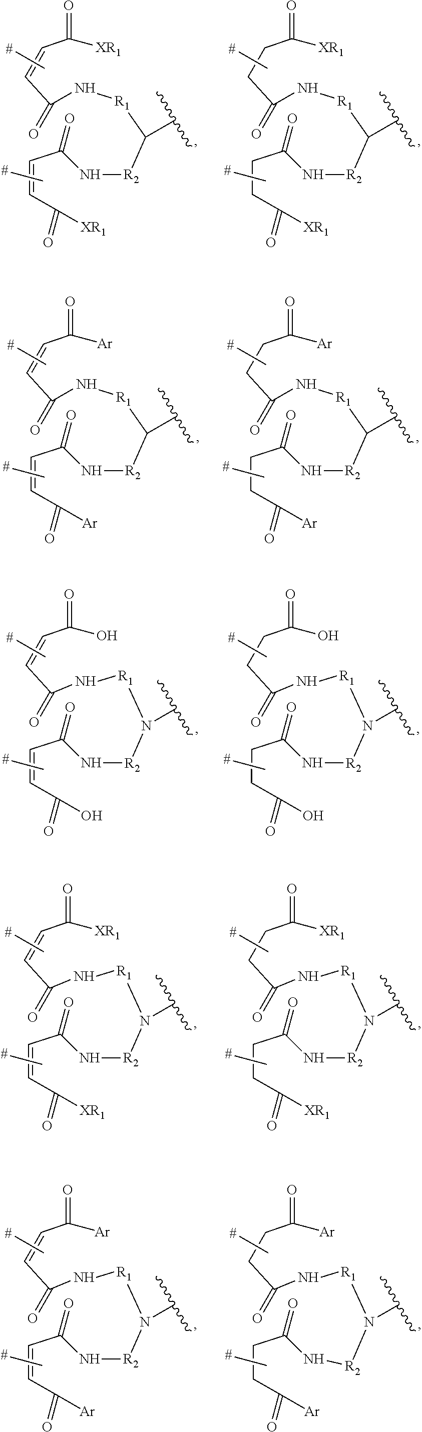

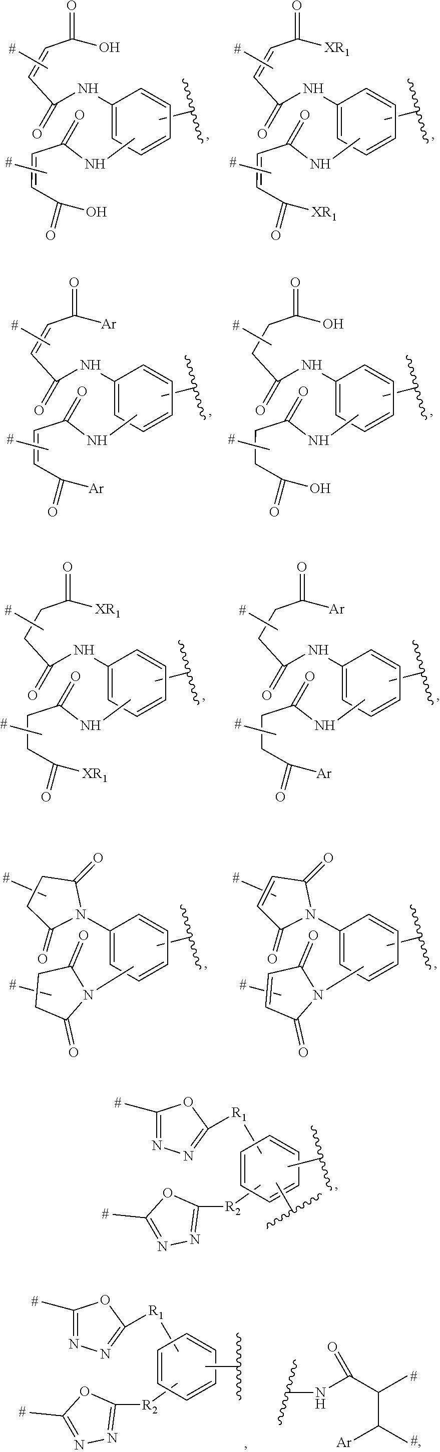

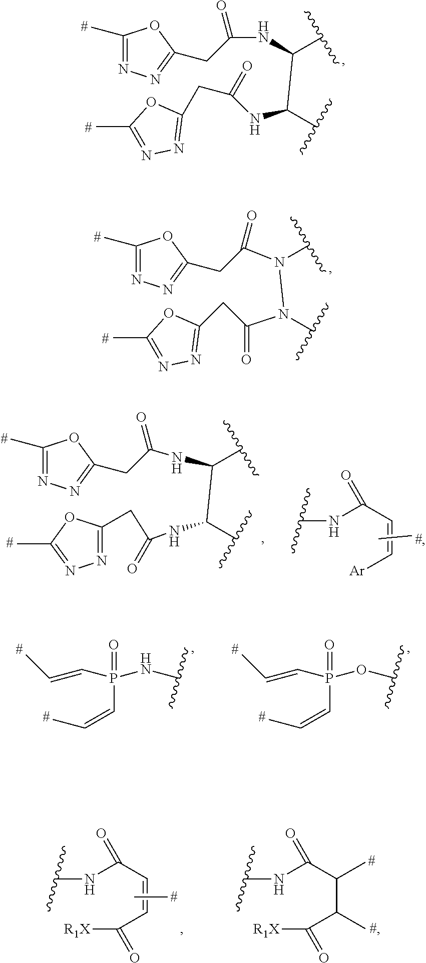

wherein “ [symbol] ” is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; wherein R₁, X_{1′} and X_{2′} are described above; X is O, NH, S, CH₂; the connecting bond “ [symbol] ” in the middle of the two atoms means it can link either one of the two atoms, Ar is an aromatic group.

More preferably, the L₁ and L₂ are independently selected from:

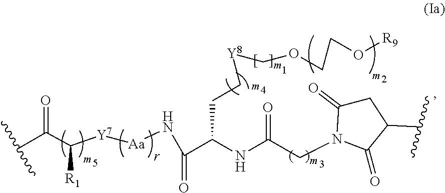

Wherein Aa is L- or D-natural or unnatural amino acids;
R₁ is H, C₁-C₈alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
R is 0-12; when r is not 0, (Aa)ᵣ is the same or different amino acids or peptide units;
m₁=1-18; m₂=1-100; m₃=1-8; m₄=0-8; m₅=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y⁸ is NHC(═O), NHS(O₂), NH(SO), NHS(O₂)NH, NHP(O)(OH)NH or C(O)NH;
R₉ is H, (O═)CR₁, (O═)CNHR₁, R₁COOH, R₁(COCH₂NH)ₘ₂H,
R₁(Aa)ᵣ or R₁(COCH₂NCH₃)ₘ₂H, wherein R₁ is defined above.

In certain embodiments, the conjugates of Formula (I), (II) and (III) are prepared via conjugation reaction of the antibody with compounds having the following formula (IV), (V) and (VI) respectively:

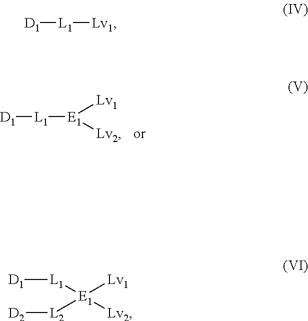

wherein: Lv₁ and Lv₂ are a reactive group, and are independently selected from:

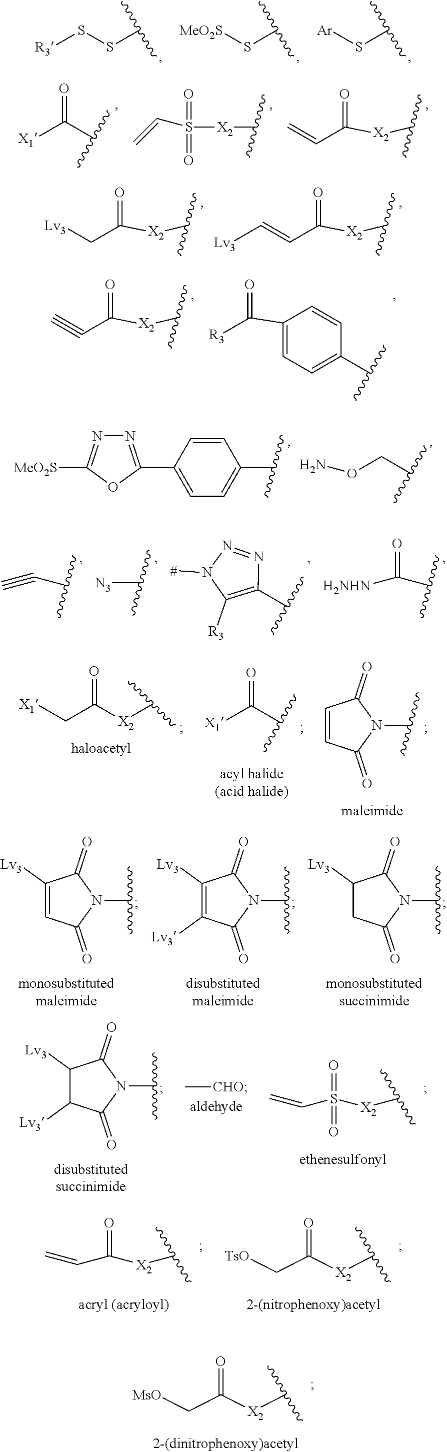

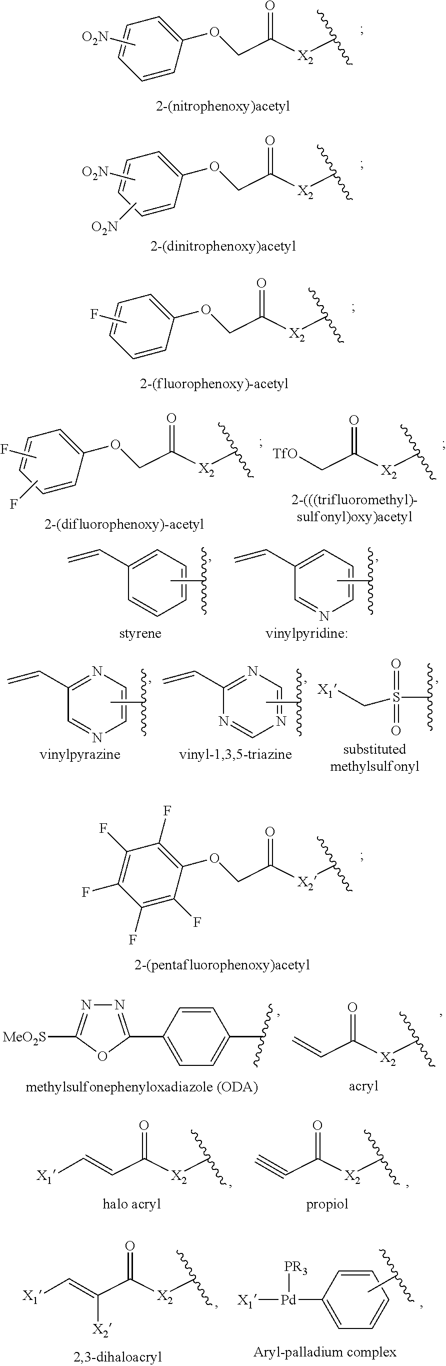

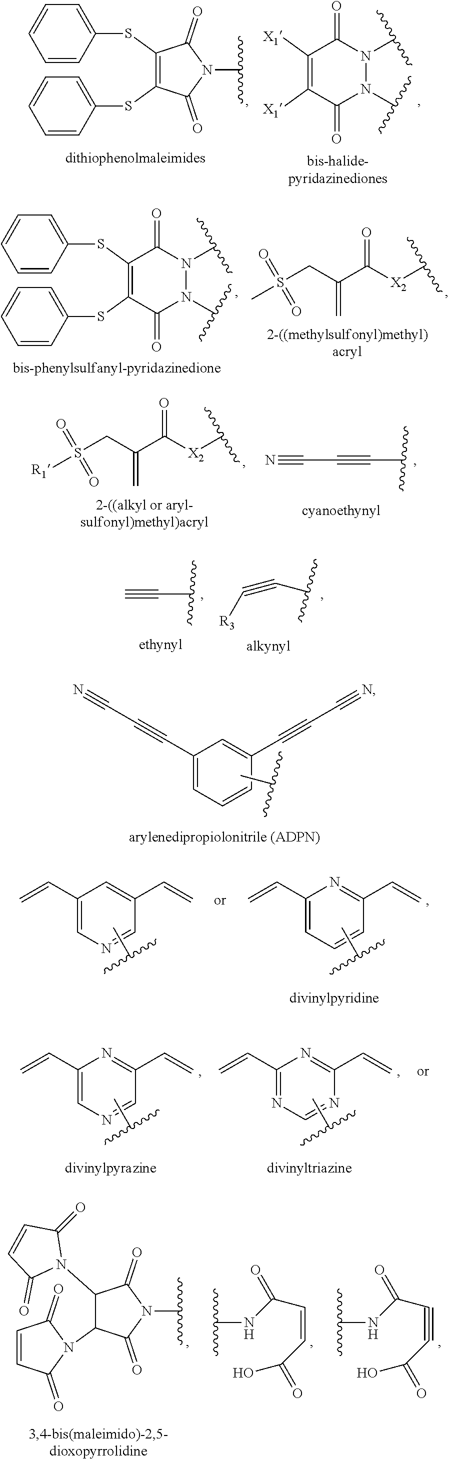

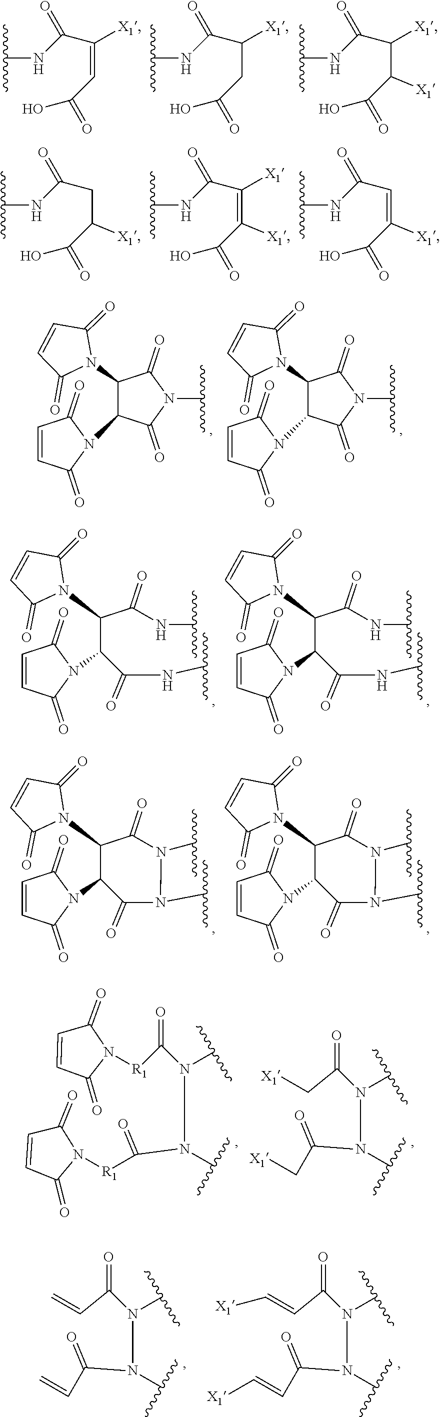

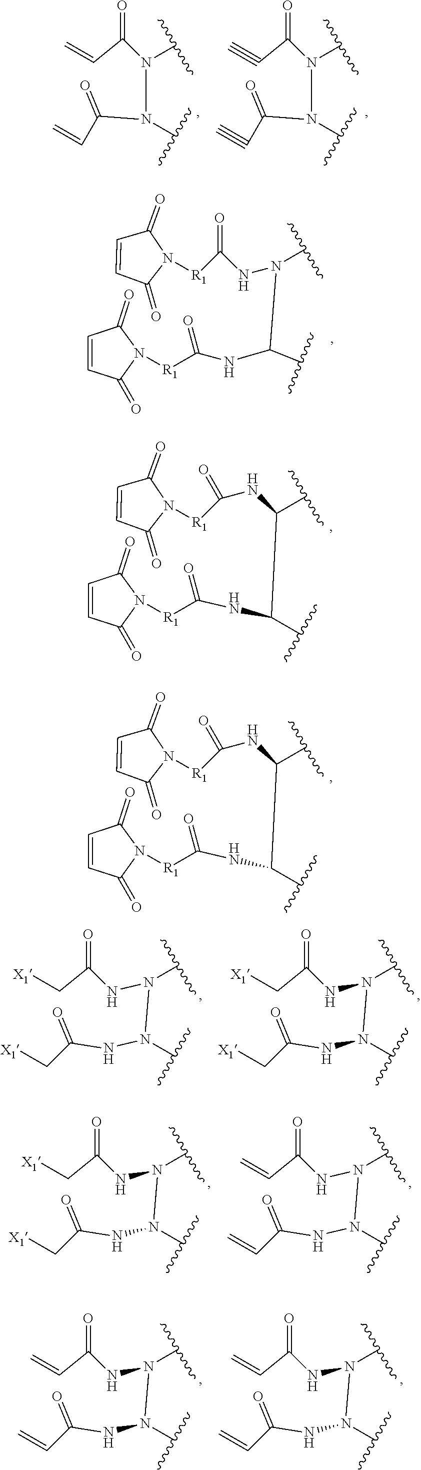

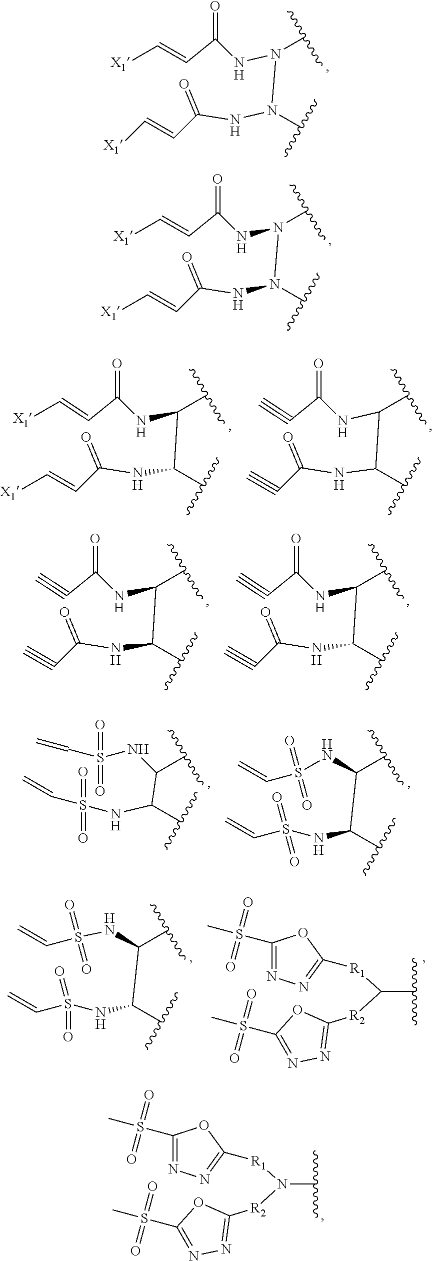

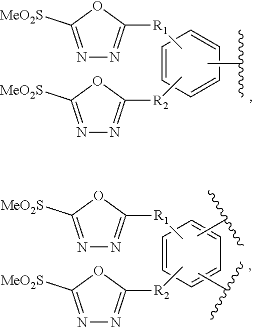

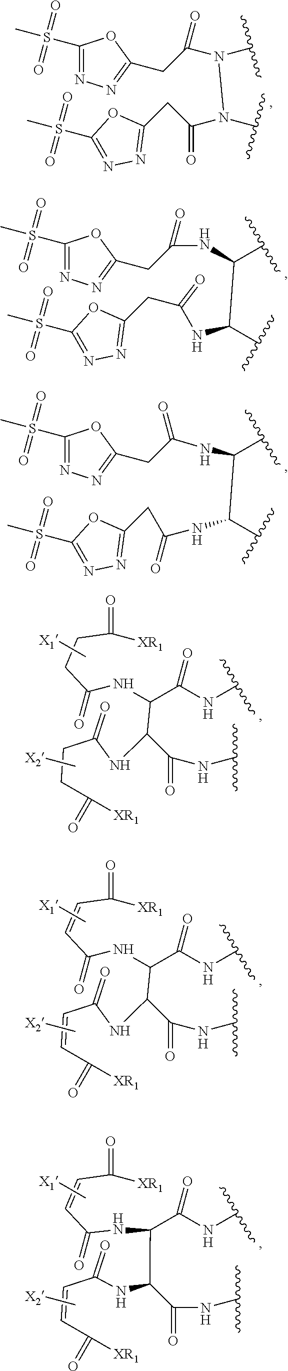

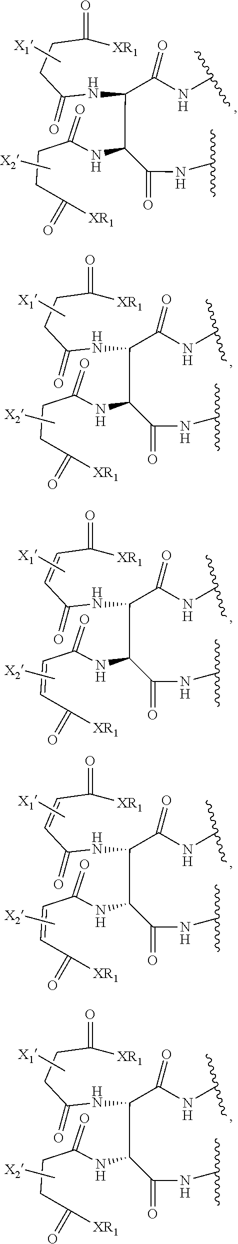

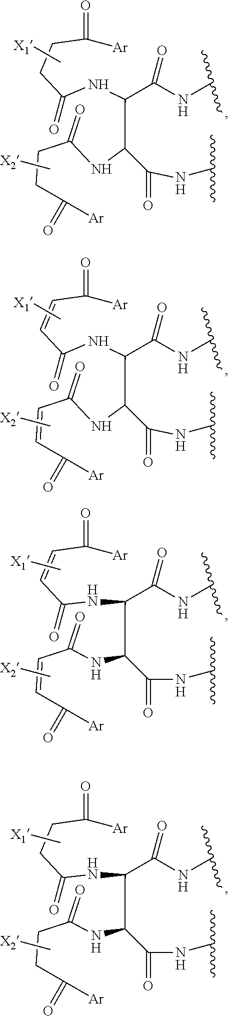

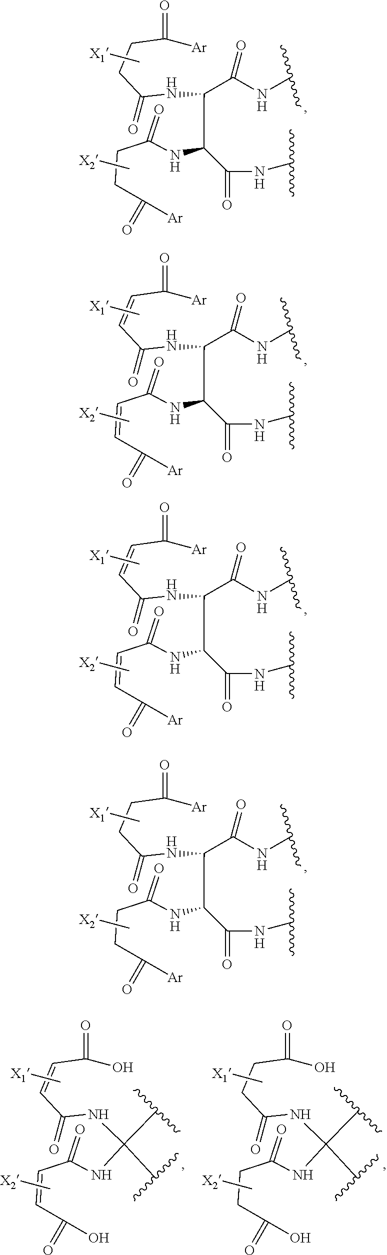

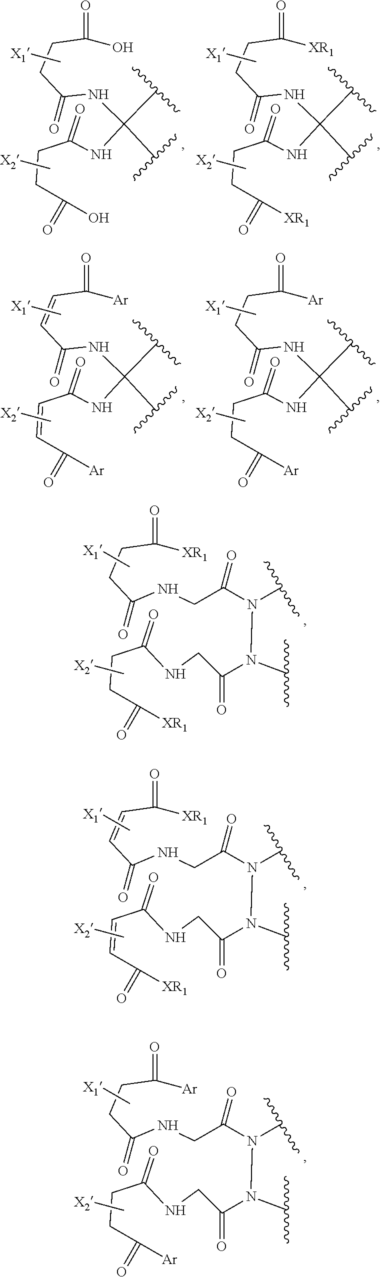

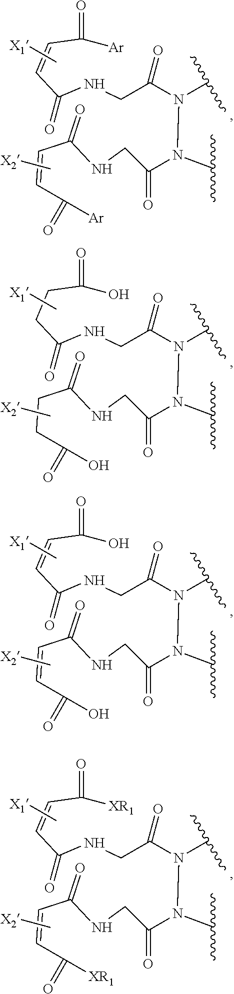

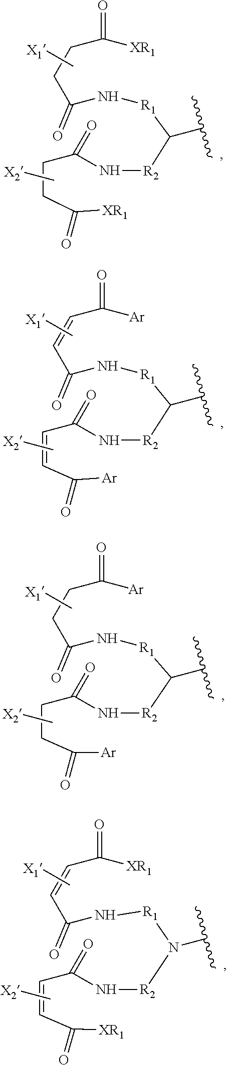

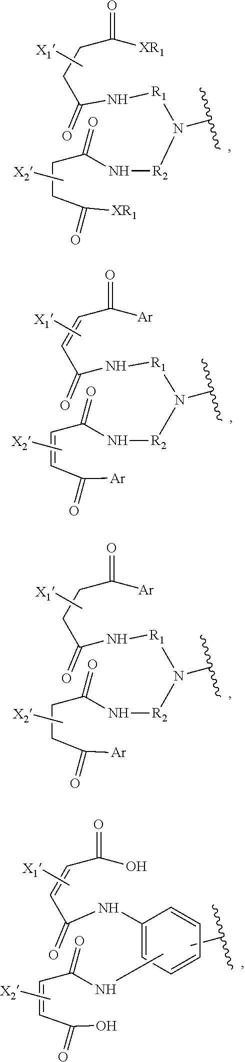

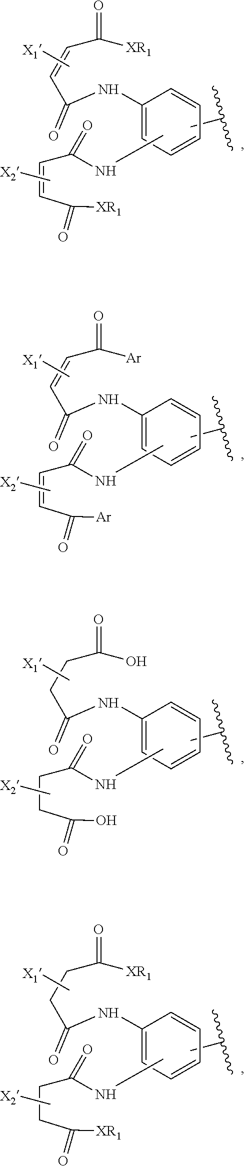

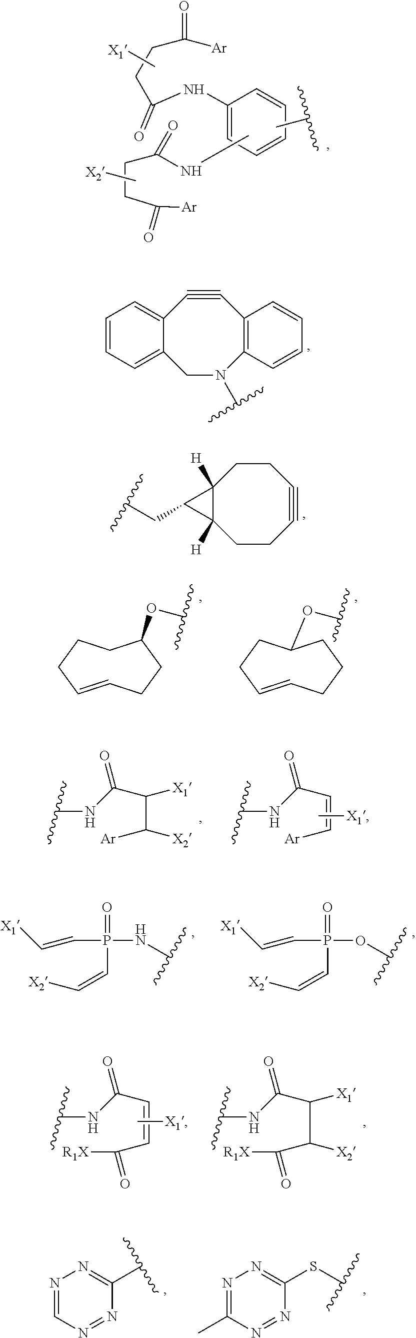

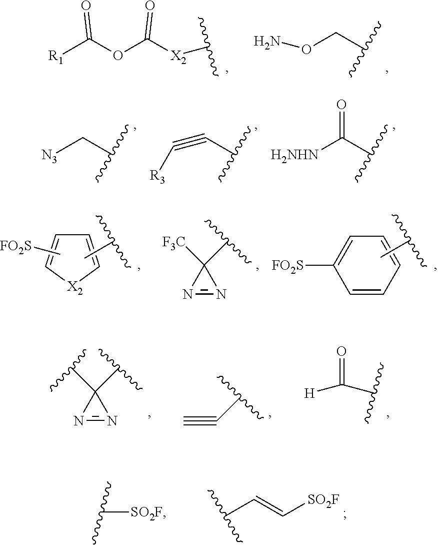

wherein X_{1′} and X_{2′} are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

In the formula (V) and formula (VI) wherein

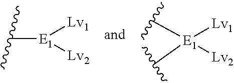

and can be selected from:

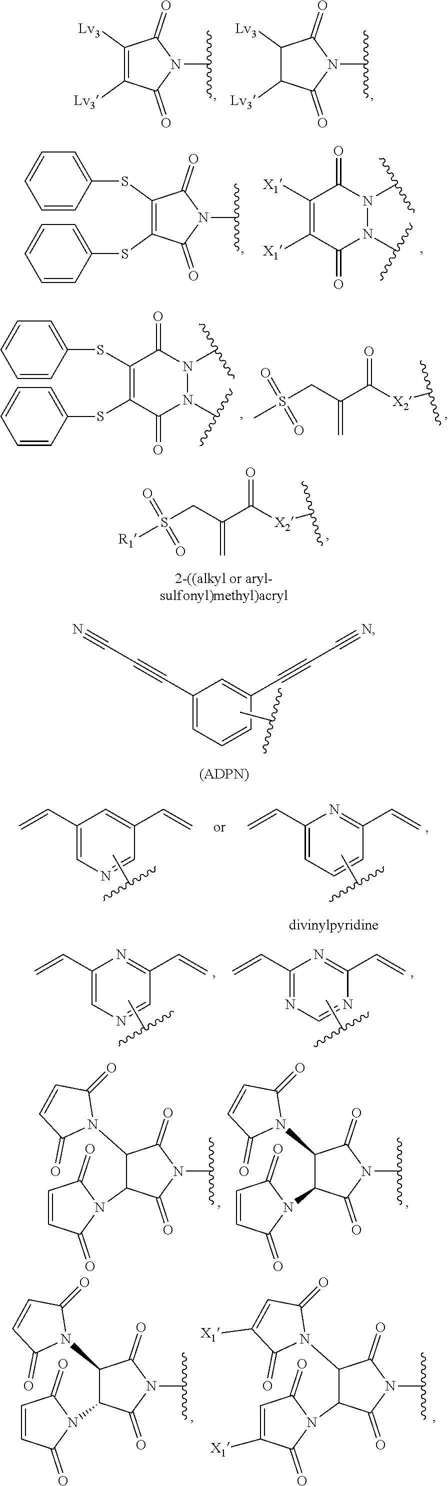

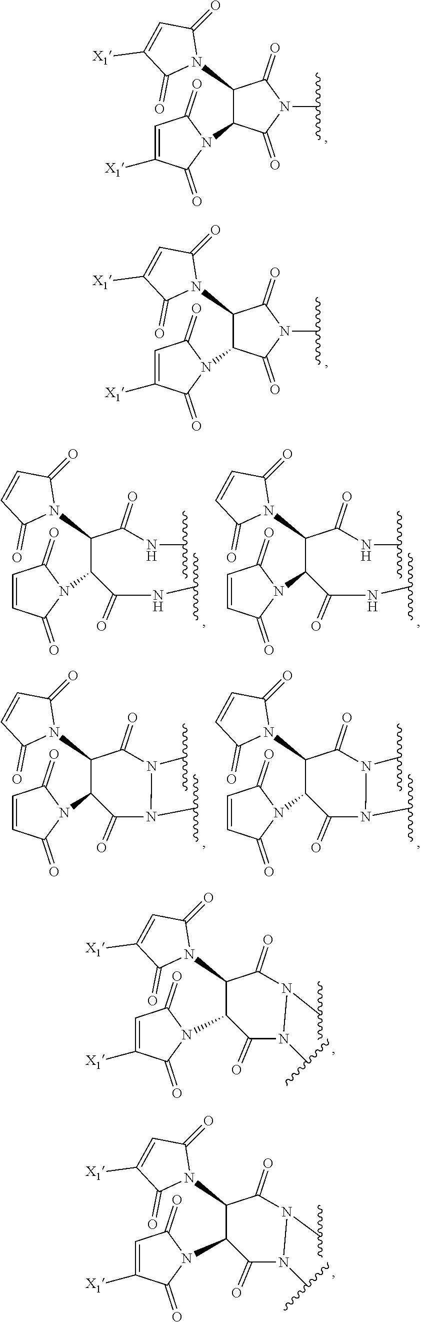

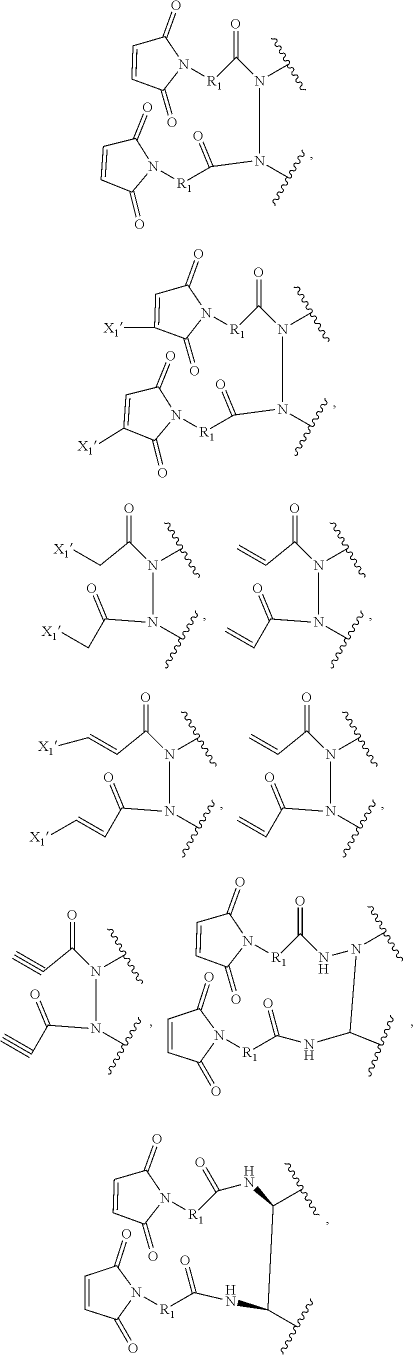

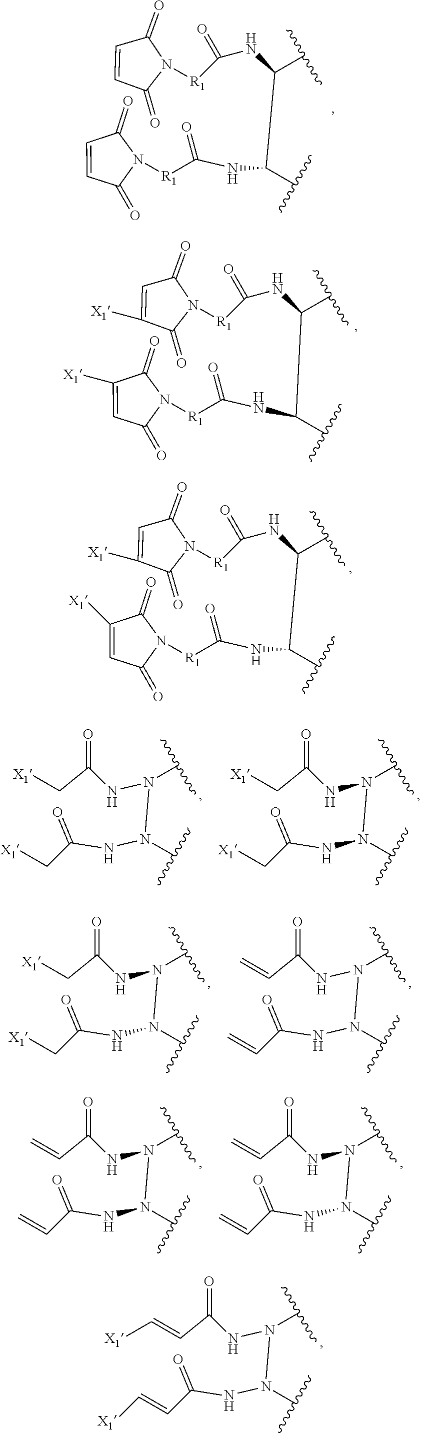

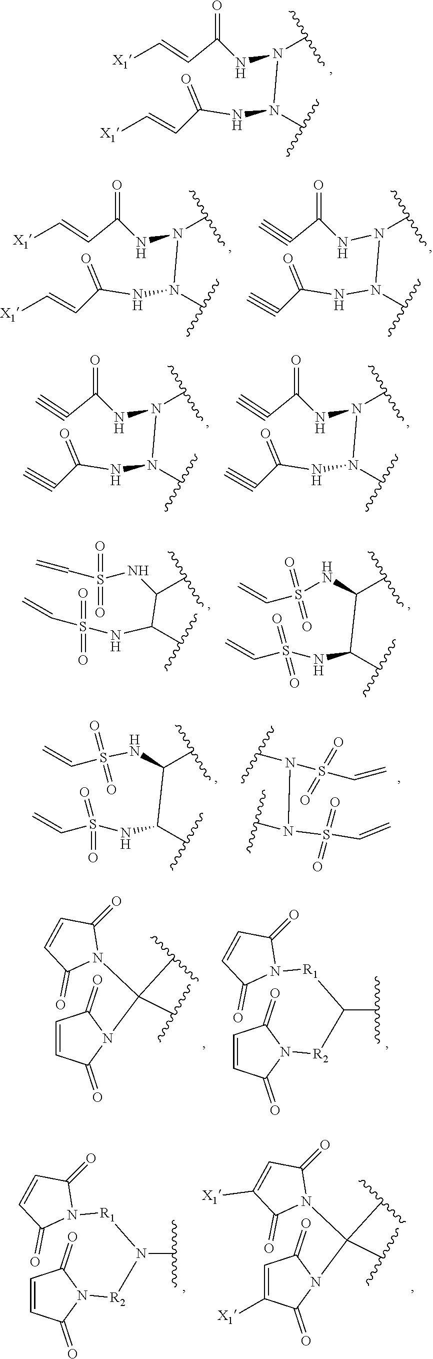

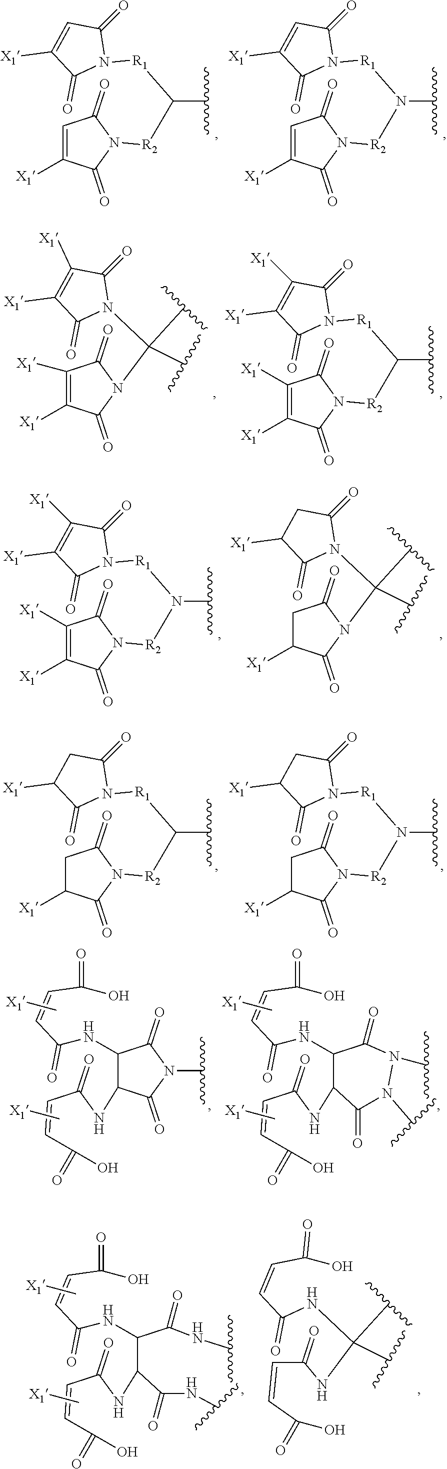

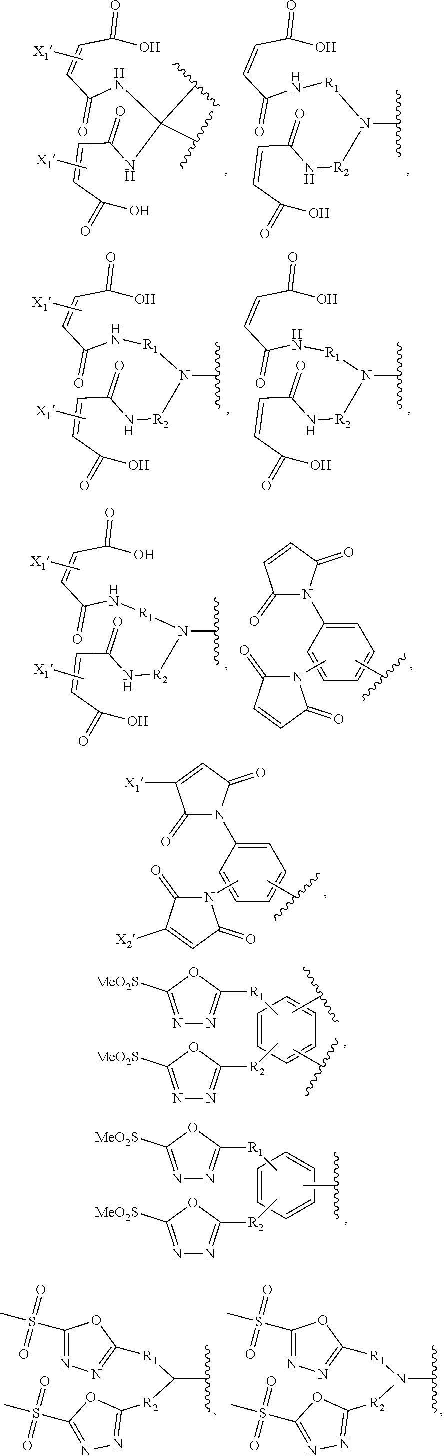

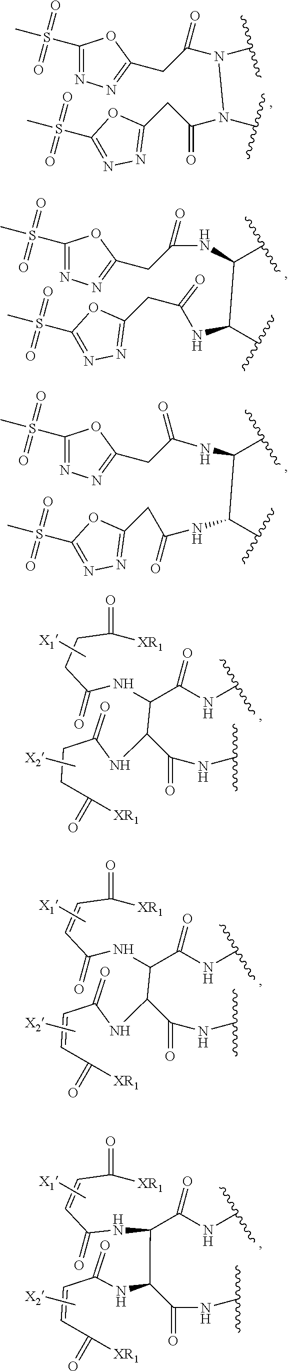

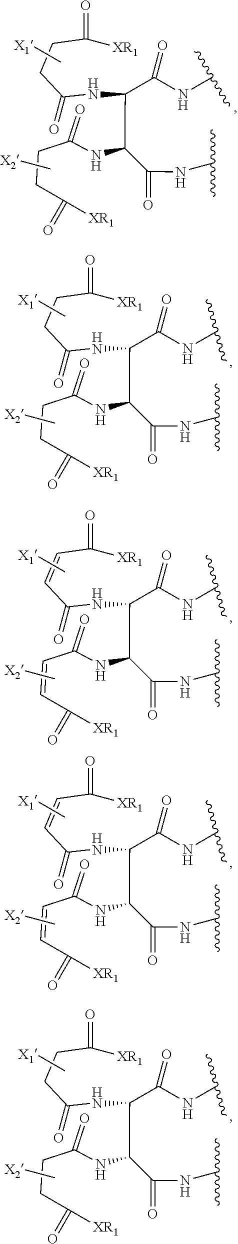

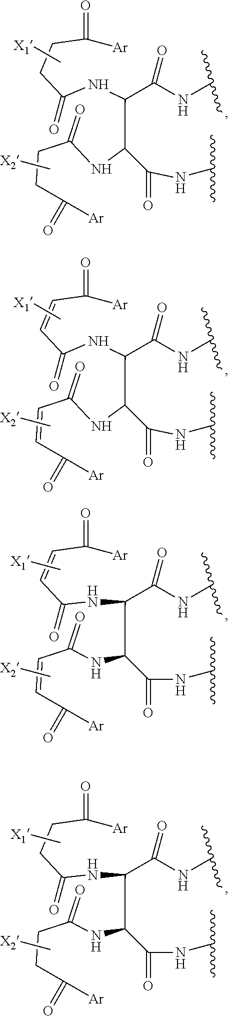

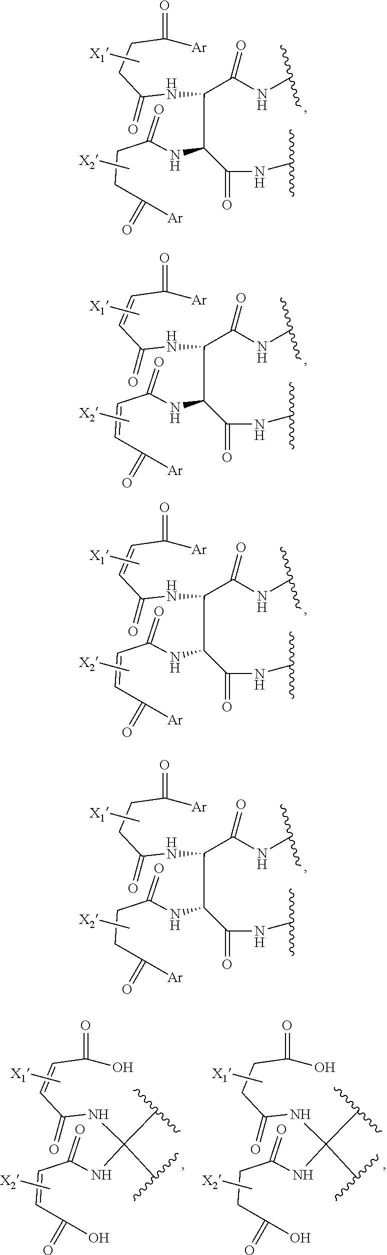

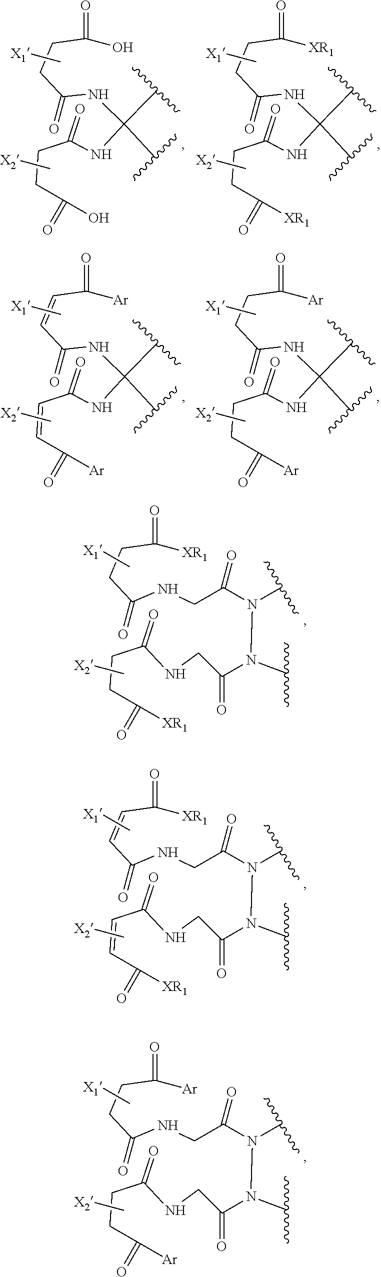

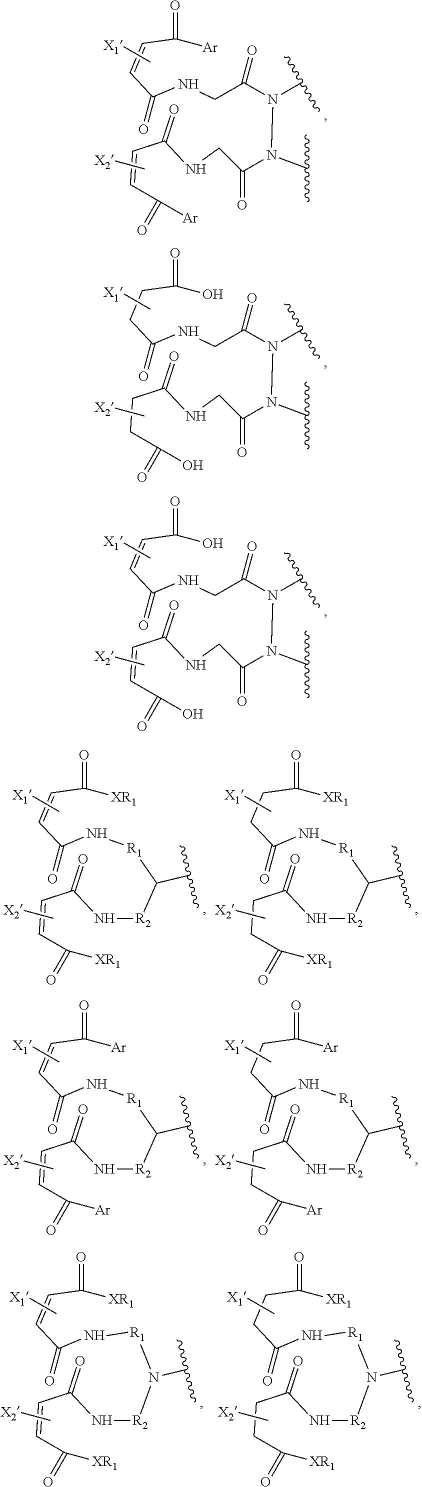

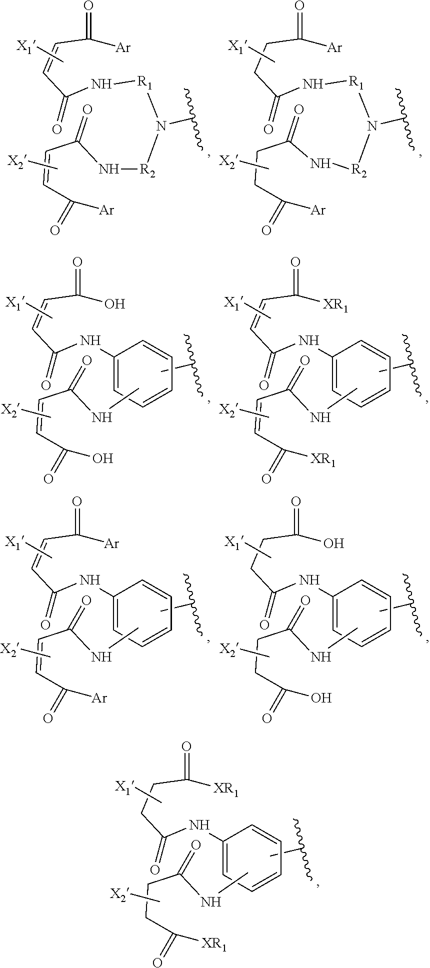

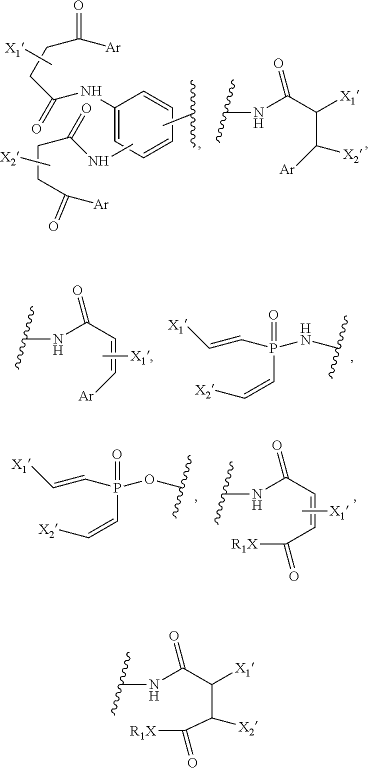

wherein Lv₃, Lv₃′, X₁′ and X₂′ are described above; the connecting bond “ [symbol] ” in the middle of the two atoms means it can link either one of the two atoms.

In some embodiments, under process of preparation of the conjugates of the present patent invention, wherein a linker having formula (VII), (VIII) or (IX) illustrated below can react first to an amino acid in the antibody independently, followed by condensation with a cytotoxic drug or cytotoxic drug/linker complex to form the conjugates of formula (I), (II), or (III):

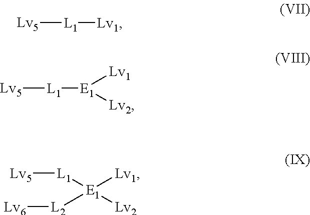

Wherein L₁, L₂, E₁, Lv₁, and Lv₂ are defined the same above for Formula (I), (II) (III). (IV), (V), and (VI); wherein Lv₅ and Lv₆ are independently selected from

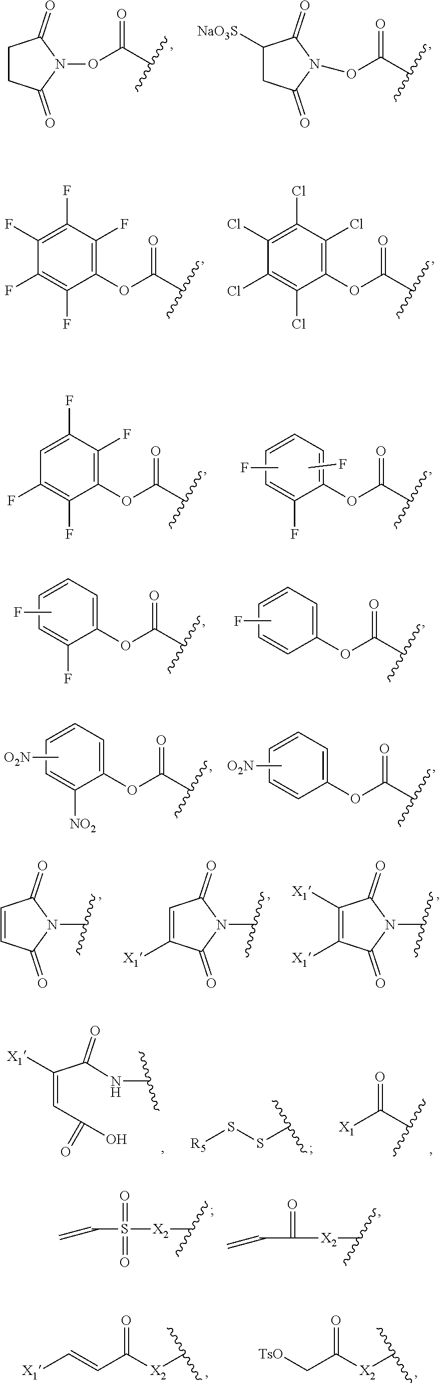

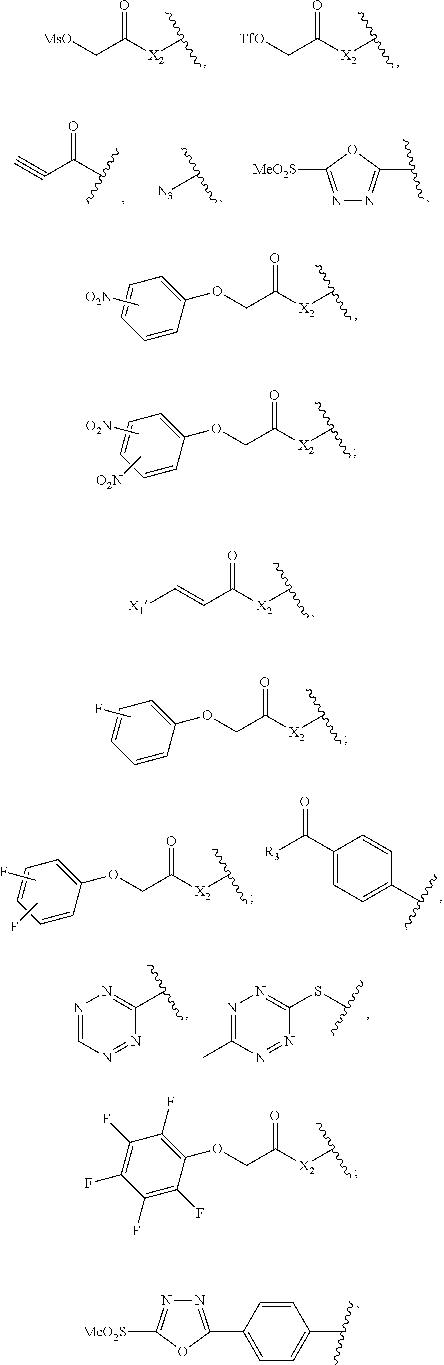

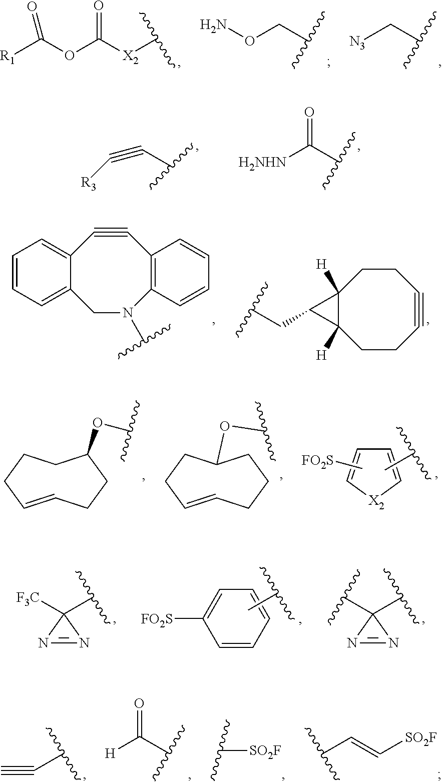

wherein X₁′ is F, Cl, Br, I, OTs (tosylate), OTf (triflate), OMs (mesylate), OC₆H₄(NO₂), OC₆H₃(NO₂)₂, OC₆F₅, OC₆HF₄, or Lv₃; X²′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions; wherein the function groups Lv₅ and/or Lv₆ can be also reacted with a thiol in a cytotoxic drug as long as the reaction are at least one fold faster or slower than the reaction between Lv₁ or Lv₂ and a thiol in an antibody, in particular, in an antibody.

In some embodiments, the conjugates of the present patent invention can be made through introduction of a certain function group in the antibody, typically generation of thiols between heavy-light chain when the antibody is IgG antibody, then the thiols simultaneously or sequentially in the conjugation process react to the linker of formula (VII), (VIII) or (IX) illustrated above to form the antibody/linker complex molecule of formula (X), (XI) or (XII) below, following by reaction with a a cytotoxic drug D₁ or D₂ independently to form the conjugate of formula (I), (II), or (III).

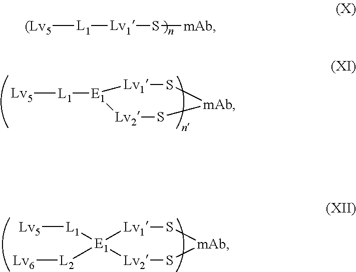

wherein Lv₅, Lv₆, L₁, L₂, E₁, Lv₁′ Lv₂′, mAb, n and n′ are described the same above.

In some embodiments, under process of the present patent invention, wherein the linker of formula (VII), (VIII) or (IX) illustrated above can react first with a cytotoxic drug to form the cytotoxic drug/linker complex molecule of formula (IV), (V) or (VI), follow by reaction with the reduced a function group in the antibody independently to form the conjugate of formula (I), (II), or (III). The first step condensation reaction of the formula (VII), (VIII) or (IX) to a cytotoxic drug can be in a separated pot, and the resulted cytotoxic drug/linker complex molecules of formula (IV), (V) or (VI) can be optionally purified by a chromatography, extraction or precipitation before for conjugation to the function group in the antibody. Normally the first step when involved in the specific reduction of disulfide bonds in an antibody, the conjugation reaction with formula (IV), (V) or (VI) is preferred in the same pot without separation of intermediates.

To distinguish the reactions between Lv₅ and/or Lv₆ to a cytotoxic drug, and Lv₁ and/or Lv₂ to a thiol in the antibody, each step of the reactions for the linker of formula (VIII), (IX) or (X) can be conducted at different conditions in the same or different reaction pots. For instance, a drug containing an amino group can undergo condensation with a carboxylic acid group in the linker in the present of a condensation regent, e. g. EDC, TBTU or BroP, to give a modified drug/linker complex of Formula (IV), (V) or (VI) bearing amide bonds. This condensation reaction can be performed at physiological buffer solution wherein the carboxylic acid group at one terminal of the linker of formula (VII), (VIII) or (IX) is activated to be N-hydoxylsuccinimidyl (NHS), pentfluorophenyl, dinitrophenyl ester, or carboxylic acid chloride group, etc, which can react to a drug bearing an amino group to provide drug/linker complex of Formula (III), (IV) or (V), then subsequently or simultaneously undergo the conjugation to thiols of the antibody to form the conjugate of formula (I), (II), or (III). In another practice, the linker of formula (VII), (VIII) or (IX) bearing both a thiol reactive group (e. g. maleimido, vinylsulfonyl, haloacetyl, acrylic, substituted propiolic) at one terminal and a drug reactive group (e. g. hydoxylsuccinimidyl (NHS), pentfluorophenyl, dinitrophenyl ester, amino, alkyloxylamino or clickable chemistry group (e. g. azide, alkyne, dibenzocyclooctyne, BCN ((1R, 8S, 9s)-bicyclo[6.1.0]non-4-yn-9-ylmethanol)) at the other terminal can undergo the conjugation to thiols of the antibody in a buffer solution at pH 4.5-7.5, 2° C.-40° C. (preferably 2° C. -8° C., more preferably 2° C. -6° C.,) with or without addition of 0˜30% of water mixable (miscible) organic solvents to form the antibody conjugate of formula (X), (XI) or (XII) independently. Then a drug bearing a reactive group matched to the reactive group in the antibody-linker conjugate of formula (X), (XI) or (XII) accordingly can be subsequently or simultaneously added to the reaction solution to provide the conjugate of formula (I), (II), or (III). In the second step reaction, the antibody--linker conjugate of formula (X), (XI) or (XII) can be optionally purified before proceeding the condensation with a drug, and the condensation condition of the second step can be adjusted, e. g. the pH can be adjusted to 6.5-8.0, and/or temperature can be adjusted to 20-45° C. if needed.

In some embodiments, during the process of the conjugation prior to conjugating with a drug, the antibody can be modified through attachment of a heterobifunctional cross linker of formula (X), (XI) or (XII), such as with linkers of Amine-to-Sulfhydryl (succinimidyl (NHS) ester/maleimide, NIS ester/pyridyldithiol, NHS esters/haloacetyl), diazirine (SDA)-to-Sulfhydryl, Azide-to-Sulfhydryl, Alkyne-to-Sulfhydryl, Sulfhydryl-to-Carbohydrate (Maleimide/Hydrazide, Pyridyldithiol/Hydrazide, haloacetyl/Hydrazide), Hydroxyl-to-Sulfhydryl (Isocyanate/Maleimide), Sulfhydryl-to-DNA (Maleimide/Psoralen, Pyridyldithiol/Psoralen, haloacetyl/Psoralen), Sulfhydryl-to-Carboxyl (Carbodiimide).

The reactive group of a drug/cytotoxic agent that reacting to a modified antibody-linker conjugate of formula (X), (XI) or (XII) to give the final conjugate can be in different ways accordingly. For example, the conjugate linked via disulfide bonds is achieved via the first step, a linker of formula (VII), (VIII) or (IX) is conjugated to the antibody at 2° C.-8° C., pH 4.5-6.0, following by a disulfide exchange between a drug containing a free thiol group and the disulfide bond ((e. g. pyridyldithio moiety) in the linker attached to the modified antibody at pH 6.5-8.0, at 20° C. -40° C. Before the addition of the drug containing a free thiol for conjugation, the excess reduction agent (e. g. TCEP, or tri(3-hydroxylpropyl)phosphine) is preferably removed from the reaction pot or quenched by addition of an azide compound (e. g. 4-(azidomethyl)benzoic acid). Synthesis of the conjugates linked via thioether is achieved by first reaction of a linker containing both thiol reactive terminals of maleimido or haloacetyl or ethylsulfonyl or substituted propiolic group to the thiols in the antibody which are reduced by the process of the present patent application at 2° C.-8° C., pH 4.5-6.5 to give the antibody-linker conjugate of formula (X), (XI) or (XII), following by reaction of a drug containing a thiol at pH 6.5-8.0, at 20° C.-40° C. to to provide the conjugate of formula (I), (II), or (III). If the same pH and/or temperature conditions are chosen for the two step reactions for thioether linked conjugates, the over four times equivalents of the linker containing dual terminal thiol reactive are used for the conjugation. It should be noted that the preferred methods of synthesis of the disulfide or thiol-ether linked conjugates are through the first chemical synthesis the drug-linker complex having disulfide or thiol-ether bonds of the formula (IV), (V) or (VI); following by reaction with the thiols in the protein (antibody) according to the process of the invention. Synthesis of conjugates bearing an acid labile hydrazone linkage can be achieved by reaction of a carbonyl group with the hydrazide moiety in the linker, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997). Synthesis of conjugates bearing triazole linkage can be achieved by reaction of a 1-yne group of the drug with the azido moiety in the linker, through the click chemistry (Huisgen cycloaddition) (Lutz, J-F. et al, 2008, Adv. Drug Del. Rev. 60, 958-70; Sletten, E. M. et al 2011, AccChem. Research 44, 666-76). Synthesis of the conjugates linked via oxime is achieved by reaction of a modified antibody containing a ketone or aldehyde and a drug containing oxyamine group. A drug bearing a hydroxyl group or a thiol group can be reacted with a modified linker of Formula (X), (XI), or (XII), bearing a halogen, particularly the alpha halide of carboxylates, in the presence of a mild base, e.g. pH 8.0˜9.5, to give a modified drug/linker complex bearing an ether or thiol ether linkage of Formula (IV), (V), or (VI). A drug containing a hydroxyl group can be condensed with a linker of Formula (X), (XI), or (XII) bearing a carboxyl group, in the presence of a dehydrating agent, such as EDC or DCC, to give ester linkage, then the subject drug/linker complex undergoes the conjugation with an antibody under the process of the present invention. A drug containing an amino group can condensate with a carboxyl ester of NHS, imidazole, nitrophenoxyl; N-hydroxysuccinimide (NHS); methylsufonylphenoxyl; dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxyben-zotriazole; tosylate; mesylate; 2-ethyl-5-phenylisoxazolium-3′-sulfonate in the antibody-linker of Formula (X), (XI) or (XII) to give a conjugate via amide bond linkage of Formula (I), (II), or (III). Many regular chemical and biochemical processes of the antibody-drug conjugation are known in the art (see, e. g. Matsuda, Y. and Mendelsohn, B. A., Expert Opin Biol Ther. 2021, 21(7): 963-975; Puthenveetil, S., Methods Mol Biol. 2020, 2078: 99-112; van Delft, F., and Lambert, J. M., ed. “Chemical Linkers in Antibody-Drug Conjugates (ADCs)”, Royal Soc. Chem. Pub., 22, Dec. 2021, ISBN 978-1-83916-263-3, doi:10.1039/9781839165153; Tumey, L. N., ed. “Antibody-Drug Conjugates, Methods and Protocols”, Springer Pub., 2020, ISBN: 978-1-4939-9929-3; Khongorzul, P. et al, Mol Cancer Res. 2020, 18(1):3-19; and many references incorporated in these books and papers).

In some embodiments, the BCMA antibody conjugates are preferably prepared via a homogenous conjugation process, which comprises the following three key steps:
(a) incubating the antibody in the presence of an effective zinc cation-amino chelate/complex (Zn(NR₁R₂R₃)ₘ₁²⁺) and a reductant (e.g. Tris(2-carboxyethyl)phosphine (TCEP)) in a buffer system (e. g. PBS, Mes, Bis-Tris, Bis-Tris Propane, Pipes, Aces, Mopso, Bes, Mops, Hepes, Tes, Pipps, Dipso, Tapso, Heppso, Tris-up, Tris-HCl, Tricine, Hepps, Gly-Gly, Bicine, Taps, Hepee, Acetates, Histidine, Citrates, MES, or Borates, etc.) to selectively reduce inter chain disulfide bonds within the antibody, to generate thiols;
(b). introducing an effective amount of linker of formula (VII), (VIII) or (IX), or payload/linker complex/assembly of (IV), (V) or (VI), bearing thiol reactive groups (e.g., a drug containing maleimide terminal) to react with the thiol groups resulted from step (a); and
(c). adding an effective amount of oxidant (e. g. dehydroascorbic acid (DHAA)) to re-oxidize unreacted thiol groups and then purifying the resulted conjugates;
(d). the step (c) can be replaced by: adding an effective amount of cystine to quench the excessive conjugation linker or linker/payload complex containing thiol reactive groups (e. g. maleimide); and simultaneously or sequentially adding an azido compound (e. g. 4-(azidomethyl)-benzoic acid) or a disulfide compound (e. g. cystine) to quench the unreacted reductant (e. g. TCEP or Tris(hydroxypropyl)phosphine). The addition of cystine to to quench the unreacted reductant (e. g. TCEP) can form a cysteine which can simultaneously quench the excessive conjugation linker or linker/payload complex containing thiol reactive groups (e. g. maleimide).
wherein R₁, R₂ and R₃ in the formula of Zn(NR₁R₂R₃)ₘ₁²⁺ are independently selected from C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; m1 is selected from 1, 2, 3, 4, 5, 6, 7 or 8; Preferably m1 is 1, 2, 3 or 4.

In addition, (NR₁R₂R₃)ₘ₁ can be form a dimer, trimer, tetramer, pentamer, or hexamer wherein these polymers are covalently linked among N, R₁, R₂ and R₃; and N, R₁, R₂ or R₃ themselve or together can form heterocyclic, carbocyclic, diheterocyclic, or dicarbocyclic rings.

The Zinc cation-amino chelate/complex, Zn (NR₁R₂R₃)ₘ₁²⁺, used in step (a) is 0.01 mM-1.0 mM in concentration, or 0.5˜20 equivalents in moles of the protein, and it can be added to the reaction solution with a water-soluble organic solvent, selected from, ethanol, methanol, propanol, propandiol, DMA, DMF, DMSO, THF, CH₃CN.

The reductant is an organic phosphine, preferably selected from Tris(2-carboxyethyl)-phosphine (TECP) or Tris(hydroxypropyl)phosphine and its use in the reaction solution is 0.02 mM-1.0 mM in concentration, or 1.0-20 equivalents in moles of the protein. The oxidant to be added in step(c) may be DHAA, Fe³⁺, I₂, Cu²⁺, Mn³⁺, MnO₂, or mixture of Fe³⁺/I⁻. The oxidant used in the reaction solution is 0.02 mM-1.0 mM in concentration, or 0.2-100 equivalents in moles of the protein. The optimum pH in the conjugation reaction is typically between about 5.0 to 8.0, and preferably, about 5.5 to 7.5. The optimum temperature in the conjugation reaction is typically between about −5 to about 40° C. and preferably, about 0 to 37° C.; more preferably about 2 to 8° C.; further preferably about 2 to 6° C. The optimum time of the conjugation reaction is typically between about 15 min to about 48 hours and preferably, about 30 min to overnight (10˜16 h), more preferably about 2 h˜6 h. The optimal reaction conditions (e. g. pH, temperature, buffer, concentrations of the reactants) of course are dependent upon specifically an antibody-like protein, a payload/linker complex, are ductant and/or Zn(NR₁R₂R₃)ₘ₁²⁺ used.

In further embodiments, under the homogenous conjugation process, the resulted conjugates of formula (I), (II), or (III) are over 75% linked to the cysteine sites between heavy-light chains of an antibody, and are less than 15% linked to the cysteine sites between heavy-heavy chains (hinge region) of an antibody. Typically, for formula (I), (II) or (III), when drug/antibody ratio (DAR) is set to be 4, the distributions in percentage of the numbers of drugs in the antibody are: D0<1%, D2<10%, D4>65%, D6<10%, D8<10%; for formula (III).

The resulted conjugate may be purified by standard biochemical means, such as gel filtration on a Sephadex G25 or Sephacryl S300 column, adsorption chromatography, ion (cation or anion) exchange chromatography, affinity chromatography (e.g. protein A column) or by dialysis (ultrafiltration or hyperfiltration (UF) and diafiltration (DF)). In some cases, a small size molecule of antibody (e.g. <100 KD) conjugated with a small molecular drug can be purified by chromatography such as by HPLC, medium pressure column chromatography or ion exchange chromatography.

In general, the conjugate of Formula (I), (II), or (III) is preferably generated from a drug/linker complex of Formula (IV), (V), or (VI), as in a one pot reaction. When a thiol reduced from an antibody reacts a thiol reactive group in the terminal of drug/linker complex of Formula (IV), (V), or (VI), the Ellman reagent can be optionally used to monitor the efficient reduction of the disulfide bonds and conjugation of the tiols through measurement of the numbers of the free thiols during the reactions. A UV spectrometry at wavelength of range 190-390 nm, preferably at 240-380 nm, more preferably at 240-370 nm is preferred to be used in assisting the reaction (via monitoring the conjugation). The conjugation reaction can be thus measured or conducted in a quartz cell or Pyrex flask in temperature control environment. The drug/protein (antibody) ratios (DAR) of the conjugates can also be measured by UV at wavelength of range 240-380 nmvia calculation of the concentrations of the drug and the protein, by Hydrophobic Interaction Chromatography (HIC-HPLC) via measurement of the integration areas of each drug/protein fragment, by Capilaryelectrophoresis (CE), and/or by LC-MS or LC-MS/MS or CE-MS (the combination of liquid chromatography (LC) or CE with mass spectrometry (MS) via measurement of both the integration areas of LC or CE and Peak intensity of MS for each drug/protein fragment). It is also noted in the conjugation process of the present invention, when a drug or a drug/linker complex is not well soluble in a water based buffer solution, up to 30% of water mixable (miscible) organic solvents, such as DMA, DMF, ethanol, methanol, acetone, acetonitrile, THF, isopropanol, dioxane, propylene glycol, or ethylene diol can be added as the co-solvent in water based buffer solution.

The aqueous solutions for the modification of the antibody are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, sucrose and salts, for example, NaCl and KCl. Other biological buffers that are used for the conjugation process are listed in the definition section. The progress of the reaction can be monitored by measuring the decrease in the absorption at a certain UV wavelength, such as at 254 nm, or increase in the absorption at a certain UV wavelength, such as 280 nm, or the other appropriate wavelength. After the reaction is complete, isolation of the modified cell-binding antibody agent can be performed in a routine way, using for example gel filtration chromatography, or adsorptive chromatography.

When disulfide exchange reaction is used for modification of the antibody, the extent of the modification can be assessed by measuring the absorbance of the nitropyridine thione, dinitropyridinedithione, pyridine thione, carboxylamidopyridinedithione and dicarboxyl-amidopyridinedithione group released via UV spectra. For the conjugation without a chromophore group, the modification or conjugation reaction can be monitored by LC-MS, preferably by UPLC-QTOF mass spectrometry, or Capilaryelectrophoresis-mass spectrometry (CE-MS). The linker compounds have diverse functional groups that can react with drugs, preferably cytotoxic agents that possess a suitable substituent. For example, the modified antibody bearing an amino or hydroxyl substituent can react with drugs bearing an N-hydroxysuccinimide (NHS) ester, the modified antibody bearing a thiol substituent can react with drugs bearing a maleimido or haloacetyl group. Additionally, the modified antibody bearing a carbonyl (ketone or aldehyde) substituent can react with drugs bearing a hydrazide or an alkoxyamine. One skilled in the art can readily determine which linker to use based on the known reactivity of the available functional group on the linkers.

### Formulation and Application

The BCMA antibody conjugates of the patent application are formulated to liquid, or suitable to be lyophilized and subsequently be reconstituted to a liquid formulation. The conjugate in a liquid formula or in the formulated lyophilized powder may take up 0.01%-99% by weight as major gradient in the formulation. In general, a liquid formulation comprising 0.1 g/L˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as sodium, potassium, ammonium, or trihydroxyethyl amino salts of citric acid, ascorbic acid, gluconic acid, carbonic acid, tartaric acid, succinic acid, acetic acid or phtalic acid; Tris, tromethamine hydrochloride, sulfate or phosphate buffer. In addition, amino acid cationic components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. The polyol in the liquid formula or in the formulated lyophilized solid can be 0.0%-20% by weight. Preferably, a nonreducing sugar, sucrose or trehalose at a concentration of about from 0.1% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearylethylimidoniumethosulfate); polyethyl glycol, polypropyl glycol, and copolymers of ethylene and propylene glycol (e.g. Pluronics, PF68 etc); etc. Preferred surfactants are polyoxyethylenesorbitan fatty acid esters e.g. polysorbate 20, 40, 60 or 80 (Tween 20, 40, 60 or 80). The concentration of a surfactant in the formulation is range from 0.0% to about 2.0% by weight. In certain embodiments, the surfactant concentration is from about 0.01% to about 0.2%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenoxyl, butyl and benzyl alcohol, alkyl parabens such as methyl or propyl paraben, catechol, resorcinol, cyclohexanol, 3-pentanol, and m-cresol. The preservative in the liquid formula or in the formulated lyophilized powder can be 0.0%-5.0% by weight. In one embodiment, the preservative herein is benzyl alcohol.

Suitable free amino acids as a bulky material, or tonicity agent, or osmotic pressure adjustment in the formulation, is selected from, but are not limited to, one or more of arginine, cystine, glycine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The amino acid in the liquid formula or in the formulated lyophilized powder can be 0.0%-30% by weight.

The formulations can optionally comprise methionine, glutathione, cysteine, cystine or ascorbic acid as an antioxidant at a concentration of about up to 5 mg/ml in the liquid formula or 0.0%-5.0% by weight in the formulated lyophilized powder; The formulations can optionally comprise metal chelating agent, e.g., EDTA, EGTA, etc., at a concentration of about up to 2 mM in the liquid formula or 0.0%-0.3% by weight in the formulated lyophilized powder.

The final formulation can be adjusted to the preferred pH with a buffer adjusting agent (e.g. an acid, such as HCl, H₂SO₄, acetic acid, H₃PO₄, citric acid, etc, or a base, such as NaOH, KOH, NH₄OH, ethanolamine, diethanolamine or triethanol amine, sodium phosphate, potassium phosphate, trisodium citrate, tromethamine, etc) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. The isotonic agent is selected from mannitol, sorbitol, sodium acetate, potassium chloride, sodium phosphate, potassium phosphate, trisodium citrate, or NaCl. In general, both the buffer salts and the isotonic agent may take up to 30% by weight in the formulation.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, (2-Hydroxypropyl)-p-cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenoxyl, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{th} edition, Gennaro, Ed., Lippincott Williams & Wilkins (2005).

A pharmaceutical container or vessel is used to hold the pharmaceutical formulation of any of conjugates of the patent application. The vessel is a vial, bottle, pre-filled syringe, pre-filled or auto-injector syringe. The liquid formula can be freeze-dried or drum-dryed to a form of cake or powder in a borosilicate vial or soda lime glass vial. The solid powder can also be prepared by efficient spray drying, and then packed to a vial or a pharmaceutical container for storage and distribution.

In a further embodiment, the invention provides a method for preparing a formulation comprising the steps of: (a) lyophilizing the formulation comprising the conjugates, excipients, and a buffer system; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media, several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as the antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 6 months at 25° C. More preferred a conjugate in either liquid or loyphilizate formulation should exhibit a shelf life of more than 12 months at 25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations should exhibit a shelf life for at least two years at −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 24 months, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8-108 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4-108 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 25 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

In some embodiment, when the reconsitituted conjugates are injected under the skin, into a muscle, or into other tissues of the body, a hyaluronidase (HAase) is preferably administered together with the conjugates. The hyaluronidase here is used as an aid in helping patient body absorb the injected conjugates. The hyaluronidase is synergistically used 20-200 unit doses, preferably in 60-160 unit doses.

The conjugates of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/triweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferrably the unit dose range is from 1 to 500 mg administered one to four times a month and even more preferably from 1 mg to 100 mg, once a week, or once a biweek, or once a triweek. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasally, particularly in the form of powders, nasal drops, or aerosols; or dermally, for example, topically in ointments, creams, lotions, gels or sprays, or via trans-dermal patches. The compositions may conveniently be administered in unit dosage form and may be prepared by any of the methods well known in the pharmaceutical art, for example, as described in Remington: The Science and Practice of Pharmacy, 21^{th} ed.; Lippincott Williams & Wilkins: Philadelphia, PA, 2005.

The formulations include pharmaceutical compositions in which a compound of the present invention is formulated for oral or parenteral administration. For oral administration, tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal. Preferred tablets contain lactose, cornstarch, magnesium silicate, croscarmellose sodium, povidone, magnesium stearate, or talc in any combination.

Liquid preparations for parenteral administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

In yet another embodiment, a pharmaceutical composition comprising a therapeutically effective amount of the conjugate of Formula (I), (II), (III), or any conjugates described through the present patent can be coadministered with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The term “coadministered,” as used herein, refers to administering one or more additional therapeutic agents and the antibody or ADC described herein, or the antibody or ADC-containing composition, sufficiently close in time such that the antibody or ADC can enhance the effect of one or more additional therapeutic agents, or vice versa. In this regard, the antibody or ADC or the composition containing the same may be administered first, and the one or more additional therapeutic agents may be administered second, or vice versa. For example, the antibody or ADC or composition containing the same may be administered in combination with other agents (e.g., as an adjuvant) for the treatment or prevention of multiple myeloma. In this respect, the antibody or ADC or antibody or ADC-containing composition can be used in combination with at least one other anticancer agent including, for example, any suitable chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer), and/or surgery. The synergistic drugs or radiation therapy can be administered prior or subsequent to administration of a conjugate, in one aspect at least an hour, 12 hours, a day, a week, biweeks, triweeks, a month, in further aspects several months, prior or subsequent to administration of a conjugate of the invention.

The synergistic agents are preferably selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinibdimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtageneciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukindiftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelinacetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogenelaherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.

In some embodiments, the disclosure also provides a composition comprising the above-described antibody or antibody-drug conjugate and a pharmaceutically acceptable (e.g., physiologically acceptable) carrier. Any suitable carrier known in the art can be used within the context of the invention. The choice of carrier will be determined, in part, by the particular site to which the composition may be administered and the particular method used to administer the composition. The composition optionally may be sterile. The compositions can be generated in accordance with conventional techniques described in, e.g., Remington: The Science and Practice of Pharmacy, 21st Edition, Lippincott Williams & Wilkins, Philadelphia, Pa. (2001).

The composition of this invention desirably comprises the antibody or ADCs in an amount that is effective to treat or prevent multiple myeloma. Thus, the disclosure provides a method of killing multiple myeloma cells, which comprises contacting multiple myeloma cells that express BCMA with the antibody or ADCs described herein, or a composition comprising the antibody or ADC described herein, whereby the antibody or ADCs binds to BCMA on the multiple myeloma cells and kills the multiple myeloma cells. The disclosure also provides use of the antibody or ADC described herein, or the composition comprising the antibody or ADC, in the manufacture of a medicament for treating multiple myeloma. As discussed herein, multiple myeloma, also known as plasma cell myeloma or Kahler's disease, is a cancer of plasma cells, which are a type of white blood cell normally responsible for the production of antibodies (Raab et al., Lancet, 374: 324-329 (2009)). Multiple myeloma affects 1˜4 per 100,000 people per year. The disease is more common in men, and for yet unknown reasons is twice as common in African Americans as it is in Caucasian Americans. Multiple myeloma is the least common hematological malignancy (14%) and constitutes 1% of all cancers. Treatment of multiple myeloma typically involves high-dose chemotherapy followed by hematopoietic stem cell transplantation (allogenic or autologous); however, a high rate of relapse is common in multiple myeloma patients that have undergone such treatment. As discussed above, BCMA is highly expressed by multiple myeloma cells.

As demonstrated herein, BCMA also is expressed on multiple myeloma stem cells. As such, the disclosure provides a method of killing multiple myeloma stem cells, which comprises contacting multiple myeloma stem cells that express BCMA with the antibody-drug conjugate described herein, or a composition comprising the ADC described herein, whereby the antibody-drug conjugate binds to BCMA on the multiple myeloma stem cells and kills the multiple myeloma stem cells. Multiple myeloma stem cells can be identified in the bone marrow of multiple myeloma patients by their surface expression of CD19 and lack of CD138 surface expression (see, e.g., Matsui et al., Blood, 103: 2332-6 (2004)). These cells are uniquely clonogenic and engraft immunodeficient mice, whereas the myeloma plasma cells, defined as CD138+CD19−, do not. Multiple myeloma stem cells also are resistant to current therapies (Matsui et al., Cancer Res., 68: 190-7 (2008)). Thus, the invention provides a method of treating a patient having or at risk of having a cancer that expresses BCMA comprising administering to the patient an effective regime of the BCMA antibody or the BCMA ADC as described above. Optionally the cancer is a hematological cancer. Optionally, the hematological cancer is a myeloma, leukemia or a lymphoma. Optionally, the hematological cancer is multiple myeloma. Optionally the hematological cancer is non-Hodgkin's lymphoma (NHL) or Hodgkin's lymphoma. Optionally, the hematological cancer is myelodysplastic syndromes (MDS), myeloproliferative syndromes (MPS), Waldenstrom's macroglobulinemia or Burkett's lymphoma.

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the antibody or ADC or the composition comprising the antibody or ADC and a pharmaceutically acceptable carrier. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the antibody or ADC to elicit a desired response in the individual. For example, a therapeutically effective amount of the ADC of the invention is an amount which binds to BCMA on multiple myeloma cells and destroys them.

A pharmacologic and/or physiologic effect of treatment may be prophylactic, i.e., the effect completely or partially prevents a disease or symptom thereof. In this respect, the inventive method comprises administering a “prophylactically effective amount” of the ADC or a composition comprising the ADC to a mammal that is predisposed to multiple myeloma. A “prophylactically effective amount” refers to an amount effective, at dosages and for periods of time necessary, to achieve a desired prophylactic result (e.g., prevention of disease onset). Therapeutic or prophylactic efficacy can be monitored by periodic assessment of treated patients. In one embodiment, the ADC described herein inhibits or suppresses proliferation of BCMA-expressing myeloma cells by at least about 10% (e.g., at least about 20%, at least about 30%, at least about 40%, at least about 50%, at least about 60%, at least about 70%, at least about 80%, at least about 90%, or at least about 100%). Cell proliferation can be measured using any suitable method known in the art, such as measuring incorporation of labeled nucleosides (e.g., 3H-thymidine or bromodeoxyuridine Brd(U)) into genomic DNA (see, e.g., Madhavan, H. N., J. Stem Cells Regen. Med., 3(1): 12-14 (2007)).

The invention of the BCMA antibody and BCMA ADCs further provides a method of treating a patient having or at risk of having an immune disorder mediated by immune cells expressing BCMA comprising administering to the patient an effective regime of any of the above described antibodies or ADCs. Optionally, the disorder is a B cell mediated disorder. Optionally, the immune disorder is rheumatoid arthritis, systemic lupus E (SLE), Type I diabetes, asthma, atopic dermitus, allergic rhinitis, thrombocytopenic purpura, multiple sclerosis, psoriasis, Sjorgren's syndrome, Hashimoto's thyroiditis, Grave's disease, primary biliary cirrhosis, Wegener's granulomatosis, tuberculosis, and graft versus host disease.

In some embodiments, the invention of the BCMA antibody and the BCMA ADCs further provides a method of treating a patient having or at risk of having a cancer, an autoimmune disease, an infectious disease, viral disease or a pathogenic infection, through administering to the patient an effective regime of any of the above described antibodies or ADCs, or any of the above described antibodies or ADCs concurrently with the other therapeutic agents such as the chemotherapeutic agent, the radiation therapy, immunotherapy agents, autoimmune disorder agents, anti-infectious agents or the other conjugates.

The targeted cancer includes, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metastatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

The autoimmune disease includes, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolyticanaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjogren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome.

The infectious disease includes, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasmaurealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The pathogenic strain includes, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraiahortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4) -Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichumcandidum, GSS prion, Giardia intestinalis, Burkholderia mallei, Gnathostomaspinigerum and Gnathostomahispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli 0157.H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugiamalayi, Lymphocytic choriomeningitis virus (LCMV), Plasmodium genus, Marburg virus, Measles virus, Burkholderia pseudomallei, Neisseria meningitides, Metagonimusyokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptesscabiei, Schistosoma genus, Shigella genus, Varicella zoster virus, Variola major or Variola minor, Sporothrixschenckii, Staphylococcus genus, Staphylococcus genus, Staphylococcus aureus, Streptococcus pyogenes, Strongyloidesstercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaeawerneckii, Trichophyton genus, Malassezia genus, Toxocaracanis or Toxocaracati, Toxoplasma gondii, Trichinella spiralis, Trichomonas vaginalis, Trichuris trichiura, Mycobacterium tuberculosis, Francisellatularensis, Ureaplasmaurealyticum, Venezuelan equine encephalitis virus, Vibrio colerae, Guanarito virus, West Nile virus, Trichosporonbeigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiellatarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosomagambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

The pathogenic viruses, includes, but not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytial virus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the BCMA antibody or ADCs of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples further illustrate the invention but, of course, should not be construed as in any way limiting its scope.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Academy of Science, unless otherwise specified. Cell culture reagents were obtained from Invitrogen Corp., unless otherwise specified. All anhydrous solvents were commercially obtained and stored in Sure-seal bottles under nitrogen. PEG compounds were purchased from Biomatrik Inc, Jiaxing, China. Some chemical compounds, when were not referred synthesis from, were provided by CROs (e. g. Wuxi Apptec, HaoyuanChemexpress, Raybow Pharma) in China. Experimental animals were purchased from National Resource Center of Model Mice via GemPharmatech. Co., Ltd, Najing, China and Shanghai SLAC Laboratory Animal Co., Ltd., Shanghai, China; T-DM1 was purchased from Roche via a pharmacy in Hong Kong, China. All other reagents and solvents were purchased as the highest grade available and used without further purification. The preparative HPLC separations were performed with VarainPreStar HPLC. HPLC analysis was conducted on Agilent 1260. The mass spectral data were acquired on a WatersXevoQTOFmass spectrum equippedwithWatersAcquityUPLC separations module and AcquityTUV detector. NMR spectra were recorded on Zhongke-niujin WNMR-I 400 MHz instrument at the Department of Chemistry of Zhejiang Sci-Tech University. Chemical shifts (δ) are reported in parts per million (ppm) referenced to tetramethylsilane at 0.00 and coupling constants (J) are reported in Hz. The elemental analysis of C, H, and/or N was provided by the Department of Chemistry of Zhejiang Sci-Tech University and conducted on Elementar UNICUBE. Quantitative analysis of metal atoms was performed on Agilent ICPOES 730 ICP-MS.

### Example 1. The Generation of a Monoclonal Antibody Directed Against B-Cell Maturation Antigen (BCMA)

Following the RIMMS immunization regime described in Kilpatrick et al., Hybridoma, 16(4): 381-389 (1997), six week old BalB/c received four rounds of subcutaneous injections of purified recombinant human (rHu) TrxA-BCMA. Mice were immunized over a course of 13 days at intervals of 2-3 days. For each round of immunization, mice were first anesthetized with isoflurane. The immunogen was emulsified in complete or incomplete Freund's adjuvant. Gold adjuvant (Sigma-Aldrich) and injected bilaterally at multiple sites. Test bleeds were collected on day 13 and assayed in antigen ELISA. Mice with good serum titers were given a pre-fusion boost intraperitoneally and sacrificed on day 17. Spleen cells were harvested and fused to myeloma cell line P3-X63-Ag8.653 following the polyethylene glycol fusion method (Roche Diagnostics) to generate stable hybridomas.

Anti-BCMA-specific hybridomas were identified by screening the hybridoma supernatants in direct binding ELISA followed by FACS on BCMA-expressing RPMI-8226-BCMA cells. Positive hybridomas were further tested for their ability to bind, internalize RPMI-8226-BCMA cells in vitro and by FACS binding to BCMA expressed on cell lines.

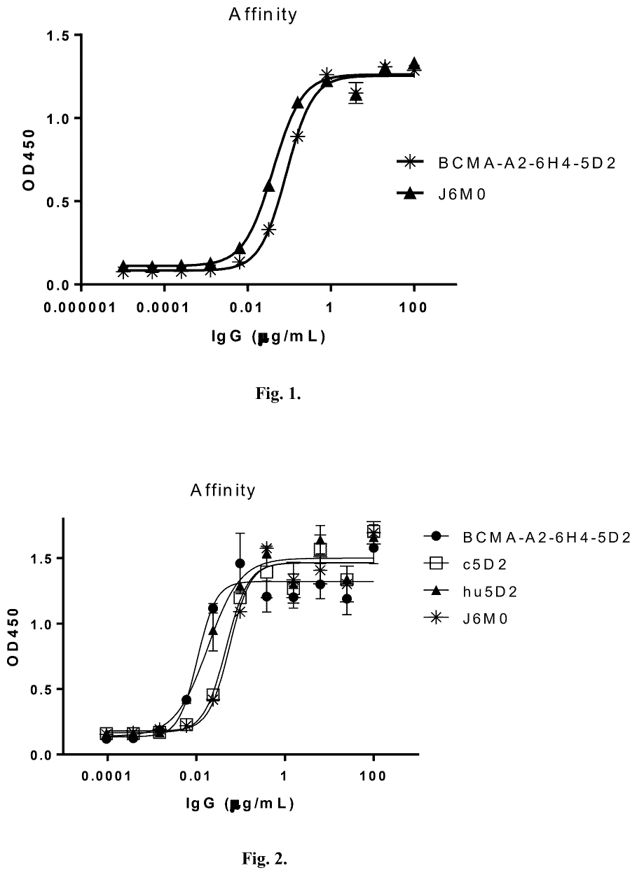

After selection and subclone, clone BCMA-A2-6H4-5D2 were selected, antibody binding affinity were determined by Elisa assay along with anti-BCMA antibody J6M0 (described in U.S. Pat. No. 9,273,141, called belantamab or DXA009B in the application), results show in FIG. 1. A deposit at China Center for Type Culture Collection (CCTCC) was made on Jun. 23, 2022 under the Budapest Treaty. The CCTCC is located at Wuhan University, Wuhan City, Hubei, Post code 430000, P. R. China. The CCTCC deposit was assigned accession number of CCTCC C2022188.

The amino acids sequences of the heavy and light chain variable regions of the monoclonal antibody BCMA-A2-6H4-5D2 are shown in Table 1.

The results of this example demonstrate the production of monoclonal antibodies directed against BCMA.

TABLE l

| SEQ ID NO | Name | Sequence |
|---|---|---|
| SEQ ID NO: 10 | BCMA-A2-6H4-5D2 VH | EVQLQQSGPELVKPGASVKMSCKASGY |
| | | TFTSFLIHWVKQKPGQGLEWIGFIIPYND |
| | | GTKYNEKFKGKATLTSDKSSSTAYMEL |
| | | SSLTSEDSAVYYCARYDGSFEGYFDVW |
| | | GAGTTLTVSSA |
| SEQ ID NO: 11 | BCMA-A2-6H4-5D2 VL | DVLMTQTPLSLPVSLRDQASISCRSSQSL |
| | | VHSDGNTYLHWYLQKPGQSPKLLIYKV |
| | | SNRFSGVPDRFSGSGSGTDFTLKISRVEA |
| | | EDLGVYFCSQSTHVPWTFGGGTKLEIK |

### Example 2. The Generation of Humanized Monoclonal Antibodies of BCMA-A2-6H4-5D2

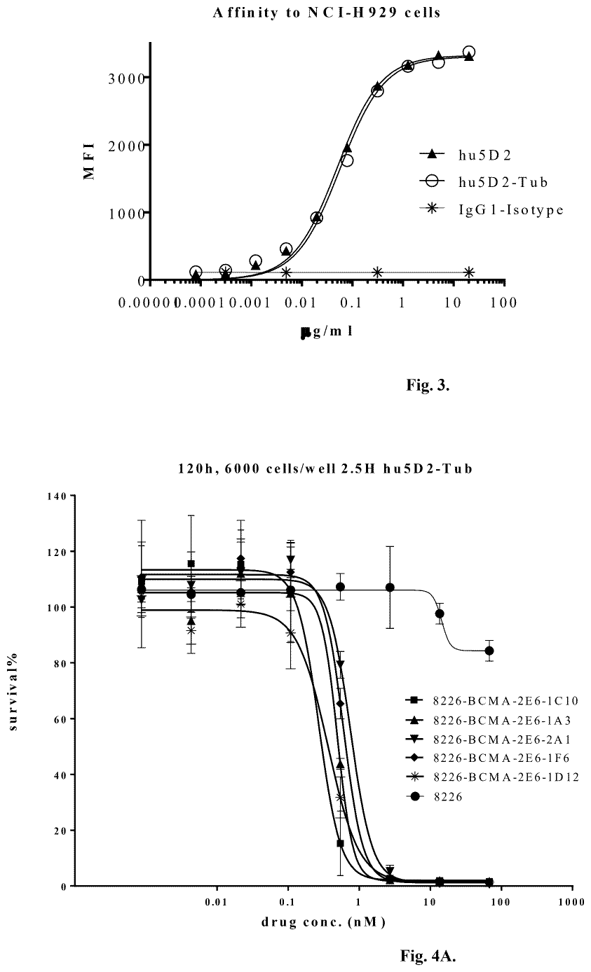

Chimeric antibody c5D2 HC (SEQ ID NO:13) constructed by fusion VH of BCMA-A2-6H4-5D2 (SEQ ID NO: 10) with human IgG1 HC constant domain (SEQ ID NO:12, which is encoded by the SEQ ID NO:24), and Chimeric antibody c5D2 LC (SEQ ID NO: 15) constructed by fusionVL of BCMA-A2-6H4-5D2 (SEQ TD NO: 11) with human Kappa LC constant domain (SEQ ID NO: 14, which is encoded by the SEQ ID NO:26). Humanized antibody hu5D2 HC (SEQ TD NO: 8) generated by substitution of corresponding Amino Acid of 5D2 with human germine line gene Amino Acid, Humanized antibody hu5D2 LC (SEQ TD NO: 9) generated by substitution of corresponding Amino Acid of 52 with human germine line gene Amino Acid. The affinity of hu5D2 showed in FIG. 2.

The amino acids sequences of the heavy and light chain variable regions of the monoclonal antibody c5D2 and hu5D2 are shown in Table 2.

TABLE 2

| SEQ ID NO | Name | Amino Acid Sequence |
|---|---|---|
| SEQ ID | c5D2 HC | EVQLQQSGPELVKPGASVKMSCKASGYTFTSFLIHWVKQKPGQ |
| NO: 13 | | GLEWIGFIIPYNDGTKYNEKFKGKATLTSDKSSSTAYMELSSLTS |
| | | EDSAVYYCARYDGSFEGYFDVWGAGTTLTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPGK |
| SEQ ID | c5D2 LC | DVLMTQTPLSLPVSLRDQASISCRSSQSLVHSDGNTYLHWYLQK |
| NO: 15 | | PGQSPKLLIYKVSNRFSGVPDRFSGSGSGTDFTLKISRVEAEDLG |
| | | VYFCSQSTHVPWTFGGGTKLEIKTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID | hu5D2 | QVQLVQSGAEVVKPGASVKMSCKASGYTFTSFLIHWVKQAPG |
| NO: 8 | HC | QGLEWIGFIIPGNGGTKYNQKFQGKATLTSDTSSSTAYMELSSL |
| | | RSEDSAVYYCARYDGSFEGYFDVWGQGTTLTVSSASTKGPSVF |
| | | PLAPSSKSTSGGTAALGCLVKDYFPEPVTVSWNSGALTSGVHTF |
| | | PAVLQSSGLYSLSSVVTVPSSSLGTQTYICNVNHKPSNTKVDKK |
| | | VEPKSCDKTHTCPPCPAPELLGGPSVFLFPPKPKDTLMISRTPEV |
| | | TCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |
| SEQ ID | hu5D2 | DVVMTQSPLSLPVSLRQPASISCRSSQSLVHSDGNTYLHWYLQK |
| NO: 9 | LC | PGQSPRLLIYKVSNRDSGVPDRFSGSGSGTDFTLKISRVEAEDLG |
| | | VYFCSQSTHWPWTFGQGTKLEIKRTVAAPSVFIFPPSDEQLKSGT |
| | | ASVVCLLNNFYPREAKVQWKVDNALQSGNSQESVTEQDSKDS |
| | | TYSLSSTLTLSKADYEKHKVYACEVTHQGLSSPVTKSFNRGEC |

### Example 3. A Traditional Method of Producing an Antibody-Drug Conjugate (ADC) Comprising a BCMA Monoclonal Antibody Conjugated to a Cytotoxin Having a Terminal of Maleimido Group

The hu5D2 monoclonal antibody was conjugated to a cytotoxin having a terminal of maleimido group. Specifically, purified antibody was incubated with a 3.2-4.2 molar excess of the reducing agent TCEP (Tris(2-carboxyethyl)phosphine) in PBS pH 7.2, 1 mM EDTA (Ethylenediamine tetraacetic acid) for 1 hours at 37° C. Subsequently, 6.5-9.0 equivalents of the payload of a cytotoxin having a terminal of maleimido group from a stock solution in 10% (v/v) DMA or DMSO was added, followed by incubation at room temperature for one hour to 3 hours under gentle rotation. The conjugation reaction was optionally quenched by the addition of four molar equivalents (over the payload) of N-acetyl cysteine. After incubation, the reducing agent and the excess payload/linker complexes were removed by 2-10. times of dialysis in PBS pH 5.0-7.2, at 4° C. using 10,000 MWCO dialysis cassettes.

The conjugation process may result in 0.1 to 10% of aggregate formation. Macromolecular aggregates, conjugation reagents, including cysteine quenched payloads, can be removed using ceramic hydroxyapatite Type II chromatography (CHT) as described e.g. Thompson et al., J. Control Release, 236: 100-116 (2016). The ADCs were optionally formulated in 25 mM Histidine-HCl, 7% sucrose, 0.02% polysorbate-20 or 80, pH 6.

To determine monomeric content, aggregates, and fragments, analytical size-exclusion chromatography (SEC-HPLC) was performed using 100 m (100 μL volume) of antibodies or ADCs, which were loaded into a TSKgel® G3000WXL column (Tosoh Bioscience, Tokyo, Japan). The mobile phase was composed of 0.1 M sodium sulfate, 0.1 M sodium phosphate, and 10% isopropanol, pH 6.8. The flow rate was 1 mL/min, and each analysis was carried out for 10-45 minutes at room temperature. Hydrophobic interaction chromatography (HIC-HPLC) was used to assess conjugation and drug load distribution, and was performed using a butyl-non porous resin (NPR) column (4.6 mm ID×3.5 cm, 2.5 μm, Tosoh Bioscience). The mobile phase A was composed of 25 mM Tris-HCl, 1.5 M (NH₄)₂SO₄, pH 8.0; and the mobile phase B was composed of 25 mM Tris-HCl and 5% isopropanol, pH 8.0. 100 μL of antibodies or ADCs at a concentration of 1 mg/mL were loaded and eluted at a flow rate of 1 mL/min with a gradient of 5% B to 100% B over 10-30 min. Reduced reverse phase chromatography (rRP-HPLC) was used to confirm chain-specific conjugation. The antibodies and ADCs were reduced at 37° C. for 20 minutes using 42 mM dithiothreitol (DTT) in PBS (pH 7.2). 10 μg of reduced antibodies or ADCs were loaded onto a polymeric reverse phase media (PLRP-S) 1000 A column (2.1×50 mm) (Agilent Technologies, Santa Clara, Calif.) and eluted at 80° C. at a flow rate of 1 mL/min with a gradient of 5% B to 100% B over 20-35 minutes (mobile phase A: 0.1% trifluoroacetic acid in water; mobile phase B: 0.1% trifluoroacetic acid in acetonitrile).

Conjugation at the heavy and light chains and drug/antibody ratios (DAR) were determined by reduced liquid chromatography mass spectrometry analysis (rLCMS) performed on an Agilent 1290 series uHPLC coupled to an Agilent 6230 TOF (Agilent Technologies, Santa Clara, Calif.). 2 μg of reduced antibodies or ADCs were loaded onto a ZORBAX® rapid resolution high definition (RRHD) 300-Diphenyl column (2.1×50 mm, 1.8 μm) (Agilent Technologies, Santa Clara, Calif.) and eluted at a flow rate of 0.5 mL/min using a step gradient of 80% B after 2.1 min (mobile phase A: 0.1% Formic acid in water and mobile phase B: 0.1% Formic acid in acetonitrile). A positive time-of-flight MS scan was acquired, and data collection and processing were carried out using MassHunter software (Agilent Technologies, Santa Clara, Calif).

### Example 4. A Method of Production of BCMA Expression Cell Lines

Stable cell lines were developed by transfecting RPMI-8226 cells with either a full-length BCMA clone or an empty vector coexpress GFP protein. Flow cytometry confirmed positive expression of BCMA on the surface of the BCMA transfected (RPMI-8226-BCMA). These cell lines were subsequently used as a tool to confirm the specificity of cloned BCMA antibodies.

### Example 5. The Binding Affinity of Monoclonal BCMA Antibodies and ADCs Described Herein to Soluble and Membrane-Bound BCMA

Binding of BCMA-A2-6H4-5D2, hu5D2 and c5D2 to soluble BCMA was determined by using Elisa assay. Elisa assay were performed coating 1 μg/ml soluble BCMA, 50 μL/Well for 1 hour at 37° C., followed by blocking with PBS+2% BSA. A range of antibody concentrations were diluted, and added to pre-coated Elisa plate for incubation for 1 hour at 37° C., followed by 3 times PBST wash (BioTek 405) and then 50 μL/Well goat Anti-Human IgG (Fab specific)-Peroxidase (Sigma-Aldrich) (1:20000 diluted) were added for detection. Before detection, plates were washed by PBST for 3 times, then TMB were added and stopped by addition of 2M H₂SO₄. The results of this experiment are shown in FIG. 2.

Binding of hu5D2, c5D2 and hu5D2-tub196 to membrane-bound human BCMA was evaluated using flow cytometry in multiple myeloma cell lines that endogenously express BCMA (NCI-H929). Binding assays were performed by incubating the anti-BCMA antibodies with 200,000 cells for 30 minutes at 4° C., followed by two washes with PBS+2% FBS (FACS Buffer). A range of antibody concentrations were evaluated using an 11-point, 4-fold dilution series. Cells were then incubated with 5 ug/mL Goat anti-Human IgG Fc Secondary Antibody, PE (Thermo Fisher Scientific) at 4° C., followed by two washes in PBS+2% FBS. Cells were resuspended in 200 uL PBS+2% FBS.

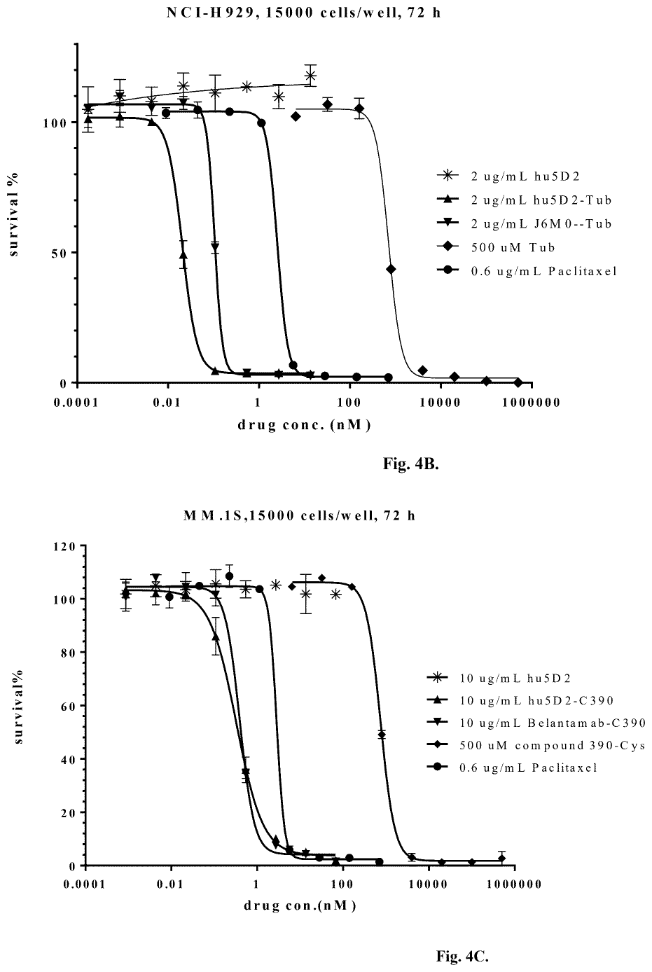

Fluorescence of live, single cells was measured using a Guava easyCyte HT cytometer. Mean fluorescence intensity values were used to determine percentage bound and EC50 was determined using Prism software. The results of this experiment are shown in FIG. 3.

### Example 6. The Methods of Killing Multiple Myeloma Cells In Vitro Using the Antibody-Drug Conjugates

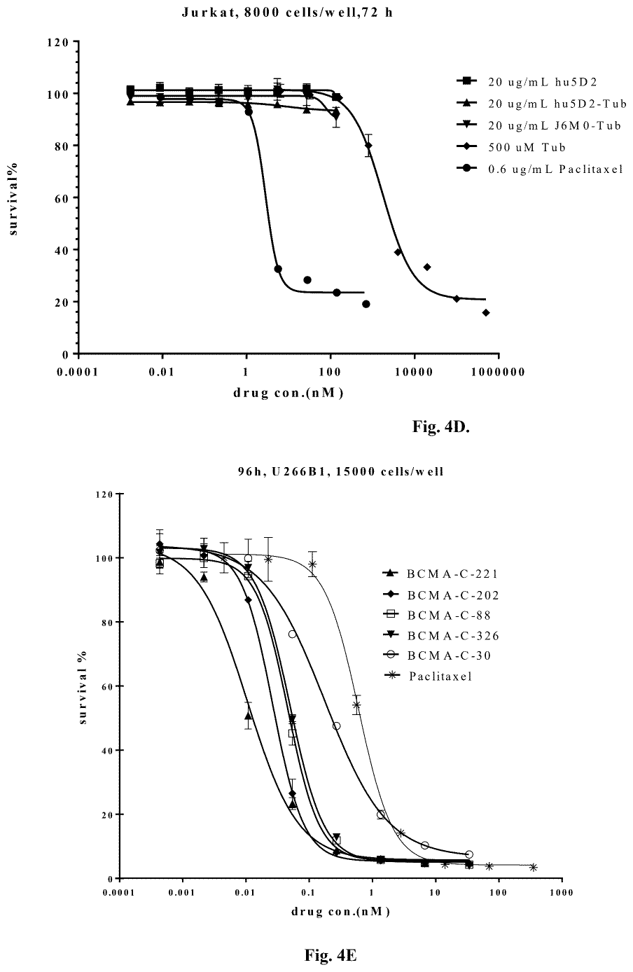

Killing of multiple myeloma and plasma cell leukemia cell lines by antibody-drug conjugates comprising hu5D2, or affinity-optimized clones thereof, conjugated to a tubulysin analog, such as compound 322 or 390, was evaluated in vitro using the protocol recommended in the CCK8 kit (Dojindo Laboratories, Japan). Briefly, 5000 cells in 180 μL RPMI+10% FBS were added to the inner wells of 96-well plates. The following BCMA-expressing cell lines were tested: RPMI-8226-BCMA, NCI-H929, MM.1S, and Jurkat also were tested. The antibody-drug conjugates were diluted to a 10× stock (100 μg/mL) in RPMI+10% FBS. Treatments were then serially diluted 1:10 in RPMI+10% FBS. 20 μL of this series was added to the cells in triplicate, resulting in a 8-point dose curve of antibody-drug conjugate ranging from 10 μg/mL at the highest concentration to 0 vg/mL at the lowest. Plates were incubated at 37° C., 5% CO₂ for 96 hours. At the end of the incubation period, 10 μL of the Substrate Solution was added to each well. The absorbance at 450 nm was measured using a SpectraMax i3x plate reader (Molecular Device, USA). Data were analyzed and graphed using GraphPad Prismor Excel software, and the half-maximal inhibitory concentration (IC₅₀) was determined. The results of this experiment are shown in FIG. 4A, 4B, 4C, 4D.

### Example 7. Glycosylation Sites Analysis for the BCMA Antibody by LC-MS

Sample Information: Recombinant humanized anti-BCMA monoclonal antibody (DXA009 DS), Batch number is 009A2201B (produced by the applicant of this patent application: Hangzhou DAC Biotechnology Co., Ltd).

Sample preparation: Glycospeptide OEQYNSTYR (SEQ ID NO: 34) with glycans attached at asparagine position. Recombinant humanized anti-BCMA monoclonal antibody (DXA009 DS, Batch number is 009A2201B), was denatured and reduced with 6M Urea, 10 mM dithiothreitol at 56° C. for about 40 min) alkylated (about 30 mM Iodoacetamide, 40 min in the dark at room temperature), diluted in 50 mM NH₄HCO₃ and digested with Trypsin (1/50, enzyme/substrate weight ratio, 4 h, 37° C.).

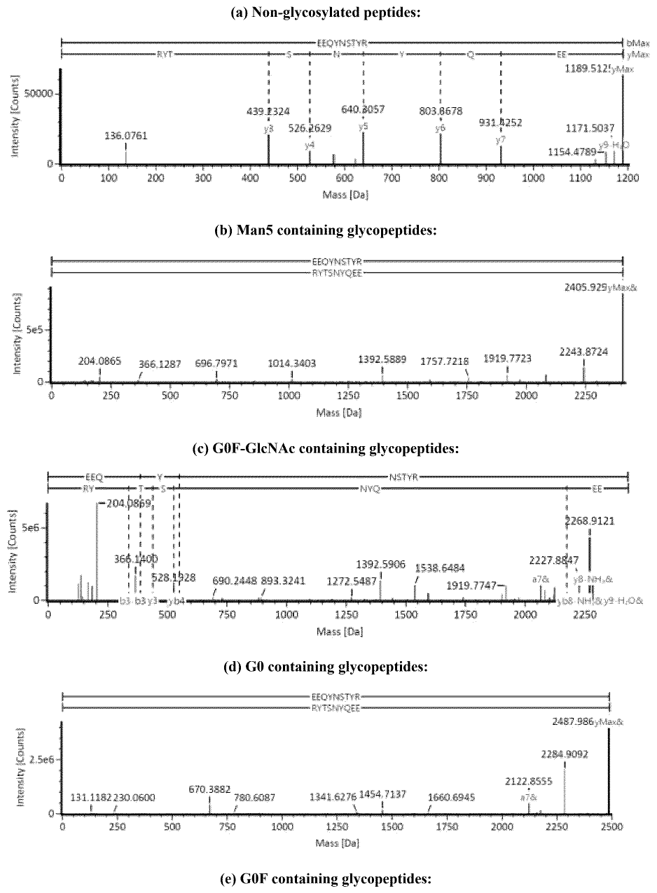

Masses and responses of the glycopeptides as illustrated in Table 3. The percentage of the glycopeptide species (including Man5, G0F-GlcNAc, G0, G0F, G1, G1F and G2F) are presented in Table 4. MS/MS daughter or product ion spectrum of glycopeptides are shown in FIGS. 5(a) -(h).

TABLE 3
Summarizing masses and responses of the glycopeptides of the BCMA antibody.

| | | Expected | Observed | Mass | | |
|---|---|---|---|---|---|---|
| Amino | | mass | mass | error | Observed | Response |
| Sequence | Glycoforms | (Da) | (Da) | (mDa) | RT (min) | (counts) |
| EEQYN(300)STYR | / | 1189.51201 | 1189.5108 | −1.2 | 12.13 | 516734 |
| EEQYN(300)STYR | Man5 | 2405.93487 | 2405.9254 | −9.4 | 11.88 | 1534614 |
| EEQYN(300)STYR | G0F- | 2430.96651 | 2430.9602 | −6.3 | 12.62 | 9127959 |
| | GlcNAc | | | | | |
| EEQYN(300)STYR | G0 | 2487.98797 | 2487.9807 | −7.2 | 12.36 | 9999319 |
| EEQYN(300)STYR | G0F | 2634.04588 | 2634.0412 | −4.7 | 12.65 | 59449512 |
| EEQYN(300)STYR | G1 | 2650.04079 | 2650.0334 | −7.4 | 12.33 | 1477878 |
| EEQYN(300)STYR | G1F | 2796.0987 | 2796.0901 | −8.6 | 12.58 | 16472720 |
| EEQYN(300)STYR | G2F | 2958.15153 | 2958.1437 | −7.9 | 12.57 | 1152395 |

Thus the position of N-glycoside is confirmed at Asn (N)-300.

TABLE 4
Summarizing the percentage of the glycopeptide
species (including Man5, G0F-GlcNAc, G0, G0F,
G1, G1F and G2F) of the BCMA antibody.
N-Glycans

| Non- | | G0F- | | | | | |
|---|---|---|---|---|---|---|---|
| glycosylate [symbol] | Man5 | GlcNA [symbol] | G0 | G0F | G1 | G1F | G2F |
| 0.52 | 1.54 | 9.15 | 10.03 | 59.61 | 1.48 | 16.52 | 1.16 |

G: Galactose;
F: Fucose;
Man5: Manose5;
GlcNAc: N-Acetylglucosamine [symbol]
indicates data missing or illegible when filed

UPLC conditions: LC system: Waters ACQUITY UPLC H-Class System, Detector: ACQUITY UPLC TUV, Absorption Wavelength: 214 nm; Trap Column: ACQUITY UPLC BEH C18 1.7 m 2.1×100 mm Column; Mobile phase A: 0.1% formic acid (FA) in water, Mobile phase B:0.1% formic acid (FA) in ACN. Performed the chromatographic separation at a flow rate of 0.25 ml/min using a linear gradient of mobile phase B (ACN with 0.1% FA) from 1% to 40% over 95 min., followed by from 40% to 80% for 10 min and then 80% to 80% for 5 min.

MS conditions: MS system: Waters Xevo-G2XS Q-TOF; Ionization mode: ESI positive, Sensitivity Mode; Data Acquisition: MS^{E}; Mass Range: m/z 100-2500 Da; Informatics: Perform the data analysis using UNIFI V1.8.2.169 Software (Waters).

### Example 8. Reduced Molecular Weight and DAR Analysis for the Deglycosylated BCMA-Tub ADCs by LC-MS

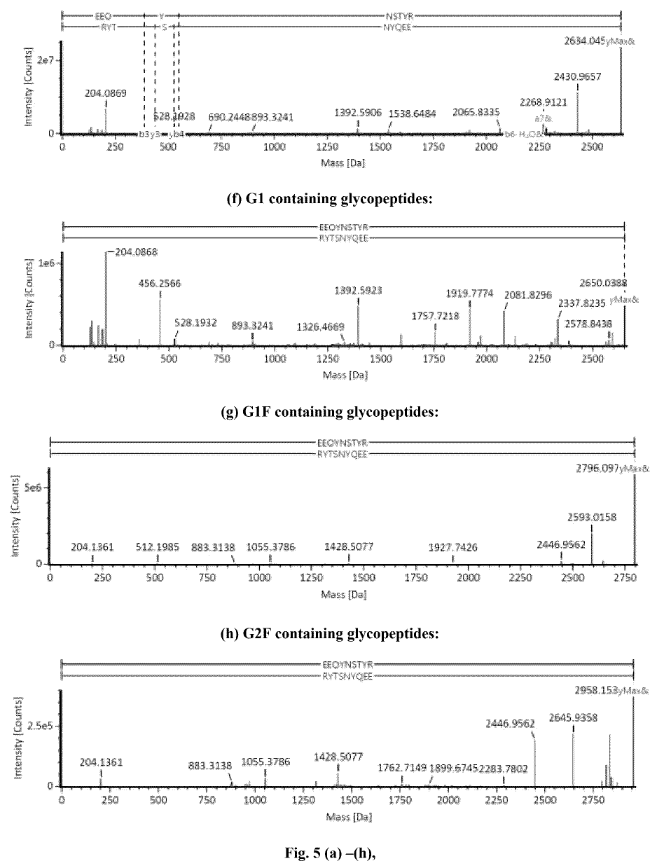

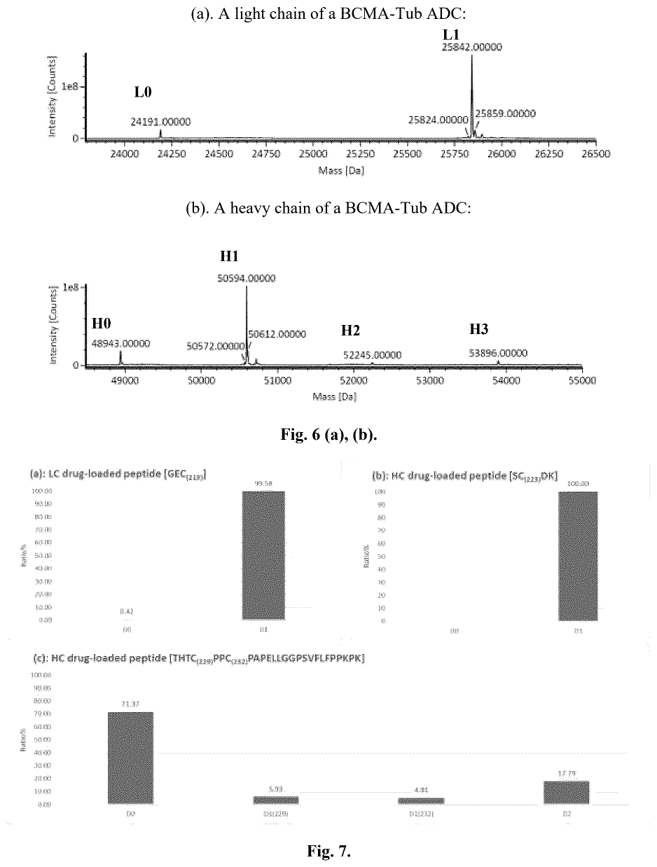

Sample preparation: Reduction of an ADC (e. g. (DXC009 DP) with 5 mM dithiothreitol at 37° C. for about 2 h, followed by a deglycosylation step with PNGase F at 37° C. overnight generated six fragments as illustrated in FIGS. 6 and 7. HC and LC existed as naked or conjugated forms carrying up to 3 payloads. The masses of each ADC fragments and the average DARs of the ADC as illustrated in Table 5. The following equation was used for average DAR calculation for conventional conjugated ADC.

TABLE 5
The summary of masses and proportions of the different ADC
fragments and the average DAR measured from peak areas.

| | Theoretical | Experimental | Delta | Distribution | Average |
|---|---|---|---|---|---|
| Species | mass (Da) | mass (Da) | (ppm) | (%) | DAR |
| L0 | 24191.6966 | 24191.463 | −9.7 | 10.60 | 3.86 |
| L1 | 25842.6633 | 25842.4693 | −7.5 | 89.40 | |
| H0 | 48942.5955 | 48943.2271 | 12.9 | 14.21 | |
| H1 | 50593.5621 | 50594.3737 | 16 | 74.65 | |
| H2 | 52244.5288 | 52245.128 | 11.5 | 4.65 | |
| H3 | 53895.4955 | 53896.2266 | 13.6 | 6.50 | |

$$AverageDAR = L 1 / ( L 0 + L 1 ) \times 2 + H 1 / ( H 0 + H 1 + H 2 + H 3 ) \times 2 + H 2 / ( H 0 + H 1 + H 2 + H 3 ) \times 2 + H 3 / ( H 0 + H 1 + H 2 + H 3 ) \times 2.$$

Method conditions: UPLC system: Waters ACQUITY UPLC H-Class System; Detector: ACQUITY UPLC TUV; Absorption Wavelength: 280 nm; Trap Column: ACQUITY UPLC C4 1.7 m 2.1×50 mm Column; Mobile phase A: 0.1% formic acid(FA) in water, Mobile phase B:0.1% formic acid (FA) in ACN; Performed the chromatographic separation at a flow rate of 0.4 ml/min using a linear gradient of mobile phase B (ACN with 0.1% FA) from 5% to 25% for 2 min, followed by 25% to 45% for 8 min, then 45% to 85% for 2 min.

MS conditions: MS system: Waters Xevo-G2XS Q-TOF; Ionization mode: ESI positive; Mass Range: m/z 500-4000 Da. Informatics: the data analysis using UNIFI V1.8.2.169 Software (Waters).

### Example 9. Drug Conjugation Site Analysis for the BCMA-ADCs by LC-MS

Sample preparation: Recombinant humanized anti-BCMA monoclonal antibody-Tubulysin B conjugate (e. g. DXC009 DP), Batch number is 22030251, Pack size is 100 mg/bottle, manufactured by Hangzhou DAC Biotechnology Co., Ltd. ADC samples were denatured and reduced (6M Urea, 10 mM dithiothreitol at 56° C. for about 40 min), alkylated (about 30 mM Iodoacetamide, 40 min in the dark at room temperature), diluted in 50 mM HEPES and digested with trypsin (1/50, enzyme/substrate weight ratio, 4 h, 37° C.).

The drug-loaded peptides of ADC as illustrated in Table 6. The masses of each ADC fragments and the average DAR as illustrated in FIG. 7. MS/MS daughter or product ion spectrum of drug-loaded peptides of the ADC as illustrated in FIG. 2.

TABLE 6
Summary of masses and responses of the drug-loaded peptides of a BCMA-ADC.

| | | | | | | Mass | | |
|---|---|---|---|---|---|---|---|---|
| | Subunit | Amino | | Expected | Observed | error | Observed | |
| Peptide | name | Sequence | Modifiers | mass (Da) | mass (Da) | (mDa) | RT (min) | Response |
| T16 | HC | SC*DK | Tub268(1) | 2102.05349 | 2102.0542 | 0.7 | 58.82 | 420219712 |
| T17 | HC | THTCPPC | Carbamido- | 2844.45754 | 2844.4677 | 10.1 | 58.61 | 439686368 |
| | | PAPELLG | methyl C(2) | | | | | |
| | | GPSVFLF | | | | | | |
| | | PPKPK | | | | | | |
| | | (SEQ ID | | | | | | |
| | | NO: 35) | | | | | | |
| T17 | HC | THTC*PP | Carbamido- | 4437.30861 | 4437.3182 | 9.6 | 72.03 | 36554152 |
| | | CPAPELL | methyl C(1), | | | | | |
| | | GGPSVFL | Tub268(1) | | | | | |
| | | FPPKPK | | | | | | |
| T17 | HC | THTCPPC | Carbamido- | 4437.30861 | 4437.3178 | 9.2 | 72.51 | 30253188 |
| | | *PAPELL | methyl C(1), | | | | | |
| | | GGPSVFL | Tub268(1) | | | | | |
| | | FPPKPK | | | | | | |
| T17 | HC | THTC*PP | Tub268(2) | 6030.15967 | 6030.1041 | −55.5 | 77.81 | 109615296 |
| | | C*PAPEL | | | | | | |
| | | LGGPSVF | | | | | | |
| | | LFPPKPK | | | | | | |
| T22 | LC | GEC | Carbamido- | 365.11255 | 365.1053 | −7.2 | 1.41 | 1951160 |
| | | | methyl C(1) | | | | | |
| T22 | LC | GEC* | Tub268(1) | 1957.96362 | 1957.9657 | 2.1 | 63.73 | 459371360 |

Method conditions: LC system: Waters ACQUITY UPLC H-Class System; Detector: ACQUITY UPLC TUV, Absorption Wavelength: 214 nm; Trap Column: ACQUITY UPLC C18 1.7 μm 2.1×100 mm Column; Mobile phase A: 0.1% formic acid (FA) in water, Mobile phase B: 0.1% formic acid (FA) in ACN; Perform the chromatographic separation at a flow rate of 0.2 ul/min using a linear gradient of mobile phase B (ACN with 0.100 FA) from 100 to 40% over 95 min., followed by 40% to 80% for 15 min.;

MS conditions: MS system: Waters Xevo-G2XS Q-TOF; Ionization mode: ESI positive, Sensitivity Mode; Data Acquisition: MS^{E}; Mass Range: m/z 100-2500 Da; Informatics: Perform the data analysis using UNIFI V1.8.2.169 Software (Waters).

### Example 10. Synthesis of meso-2,3-bis((2,4-dimethoxybenzyl)amino)succinic acid (2)

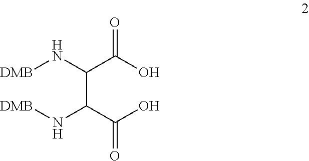

To a solution of meso-2,3-dibromosuccinic acid (500 g, 1.80 mol) in ethanol (3.6 L) was added trimethylamine (729 g, 7.20 mol), followed by 2,4-dimethoxybenzylamine (903 g, 5.4 mol). After completion of addition, the mixture was heated to 90° C. and stirred under reflux overnight. The mixture was cooled to r.t. and the formed solid was filtered, rinsed with ethanol and dried to give meso-2,3-bis(2,4-dimethoxybenzylamino) succinic acid (600 g, 72% yield).

### Example 11. Synthesis of meso-2,3-diaminosuccinic acid (3)

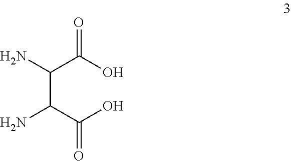

A solution of meso-2,3-bis(2,4-dimethoxybenzylamino)succinic acid (800 g, 1.78 mol) in dichloromethane (100 mL) was treated with trifluoroacetic acid (2035 g, 17.8 mol) at r.t. overnight. The mixture was concentrated and then 1N NaOH (6 L) was added slowly and stirred for 30 min. The precipitate was filtered off and the solution was adjusted to pH 5-6 using aq. HCl. The resulting white precipitate was collected by filtration, rinsed with water and dried to give meso-2,3-diaminosuccinic acid (217 g, 78% yield).

### Example 12. Synthesis of meso-2,3-bis(((benzyloxy)carbonyl)amino)succinic acid (4)

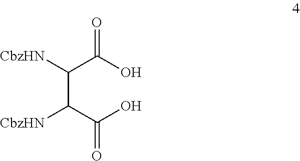 [SMILES: O=C(NC(C(=O)O)C(NC(=O)OCc1ccccc1)C(=O)O)OCc1ccccc1]

Meso-2,3-diaminosuccinic acid (386 g, 2.6 mol) was dissolved in 2 N NaOH (6.5 L) and mixed with 1,4-dioxane (2.1 L). The solution was cooled to 0° C. and benzyl chloroformate (1333 g, 7.8 mol) was added in a rate to maintain the internal temperature of below 5° C. After completion of the addition, the mixture was stirred for 3 h, warmed to r.t. and stirred overnight. The reaction was diluted with water (2 L) and washed with ethyl acetate (2×1 L). The aqueous layer was acidified with con. HCl until pH 3 was reached, and then extracted with ethyl acetate (3×2 L). The organic phase was combined and washed with water (1 L), dried over anhydrous Na₂SO₄, filtered and concentrated. The residue was triturated with dichloromethane/petroleum ether (1:1), filtered to give meso-2,3-bis(((benzyloxy)carbonyl) amino) succinic acid (900 g, 83%).

### Example 13. Synthesis of di-tert-butyl 4,4′-((2,3-bis(((benzyloxy)carbonyl)amino)succinyl) bis(azanediyl))dibutyrate (6)

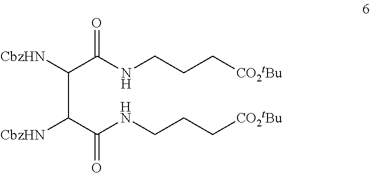 [SMILES: CC(C)(C)OC(=O)CCCNC(=O)C(NC(=O)OCc1ccccc1)C(NC(=O)OCc1ccccc1)C(=O)NCCCC(=O)OC(C)(C)C]

To a solution of compound 4 (10 g, 28.7 mmol) and tert-butyl aminobutyrate hydrochloride (11.2 g, 57.4 mol) in tetrahydrofuran (200 mL) were added HATU (32.8 g, 86.3 mmol) and diisopropylethylamine (19 mL, 115 mmol), and the reaction was stirred at r.t. overnight, diluted with water (400 mL), stirred for 10 minutes, and filtered, dried in an oven to give a white solid (17.7 g, >100% yield). MS-ESI (m/z): [M+H]⁺ calcd for C₃₆H₅₁N₄O₁₀, 699.35; found, 699.35.

### Example 14. Synthesis of di-tert-butyl 4,4′-((2,3-diaminosuccinyl)bis(azanediyl)) dibutyrate (7)

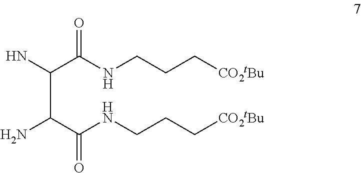 [SMILES: CC(C)(C)OC(=O)CCCNC(=O)C(N)C(N)C(=O)NCCCC(=O)OC(C)(C)C]

Compound 6 (16 g, 22.9 mmol) was dissolved in methanol (200 mL), and then Pd/C (2.0 g) was added. The reaction flask was evacuated and back-filled with hydrogen, heated to 60° C., and stirred until completion of the reaction. Filtration and concentration gave product 7 (9.8 g, 100% yield). MS-ESI (m/z): [M+H]⁺ calcd for C₂₀H₃₉N₄O₆, 431.28; found, 431.28.

### Example 15. Synthesis of Compound 9

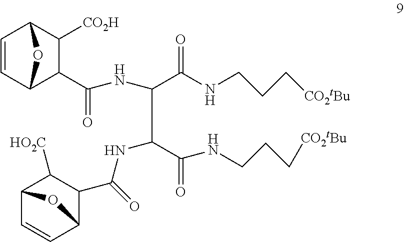 [SMILES: CC(C)(C)OC(=O)CCCNC(=O)C(NC(=O)C1C(C(=O)O)[C@H]2C=C[C@@H]1O2)C(NC(=O)C1C(C(=O)O)[C@H]2C=C[C@@H]1O2)C(=O)NCCCC(=O)OC(C)(C)C]

Compound 7 (9.8 g, 22.8 mmol) was dissolved in dichloromethane (200 mL), and exo-3,6-epoxy-1,2,3,6-tetrahydrophthalic anhydride (7.5 g, 45.5 mmol) and triethylamine (6.3 ml, 45.5 mmol) were added. The reaction was stirred at r.t. until completion, and then concentrated to dryness to give a white foamy solid (17.3 g, 100% yield). MS-ESI (m/z): [M+H]⁺ calcd for C₃₆H₅₁N₄O₁₄, 763.33; found, 763.33.

### Example 16. Synthesis of di-tert-butyl 4,4′-((2,3-bis((4R,7S)-1,3-dioxo-1,3,3a,4,7,7a-hexahydro-2H-4,7-epoxyisoindol-2-yl)succinyl)bis(azanediyl))dibutyrate (10)

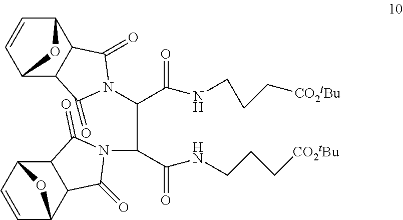 [SMILES: CC(C)(C)OC(=O)CCCNC(=O)C(C(C(=O)NCCCC(=O)OC(C)(C)C)N1C(=O)C2C(C1=O)[C@H]1C=C[C@@H]2O1)N1C(=O)C2C(C1=O)[C@H]1C=C[C@@H]2O1]

To a solution of compound 9 (17.3 g, 22.7 mmol) dissolved in DMF (300 mL), were added EDC (13 g, 68.2 mmol), HOBt (9.2 g, 68.2 mmol), and DBU (10.4 g, 68.2 mmol) slowly. The mixture was heated to 60° C. and stirred for 5 hours, cooled to r.t. and poured into water (1 L), extracted with dichloromethane (3×200 mL). The combined organic phases were washed with 2 N HCl (100 mL), brine (100 mL), dried over sodium sulfate, filtered and concentrated. The residue was triturated with petroleum ether/ethyl acetate (200 mL/500 mL), and the white solid was filtered off. The filtrate was concentrated and purified by a silica gel column to give a white foamy solid (8.2 g, 49% yield). MS-ESI (m/z): [M+H]⁺ calcd for C₃₆H₄₇N₄O₁₂, 727.31; found, 727.31.

### Example 17. Synthesis of di-tert-butyl 4,4′-((2,3-bis(2,5-dioxo-2,5-dihydro-1H-pyrrol-1-yl)succinyl)bis(azanediyl))dibutyrate (11)

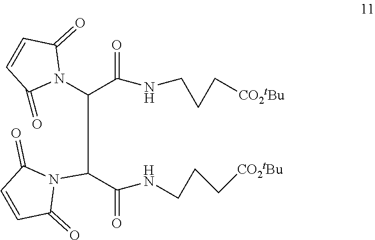 [SMILES: CC(C)(C)OC(=O)CCCNC(=O)C(C(C(=O)NCCCC(=O)OC(C)(C)C)N1C(=O)C=CC1=O)N1C(=O)C=CC1=O]

Compound 10 (8.2 g, 11.2 mmol) was dissolved in a mixture of toluene (80 mL) and DMF (80 mL), heated to 120° C. and stirred under reflux for 4 hours. The reaction was then cooled to r.t. and stirred for more than 30 minutes, and a white solid precipitated, which was then collected by filtration and dried to give the desired product (3.0 g, 45% yield). MS-ESI (m/z): [M+H]⁺ calcd for C₂₈H₃₉N₄O₁₀, 591.26; found, 591.26.

### Example 18. Synthesis of 4,4′-((2,3-bis(2,5-dioxo-2,5-dihydro-1H-pyrrol-1-yl)succinyl)bis(azanediyl))dibutyric acid (12)

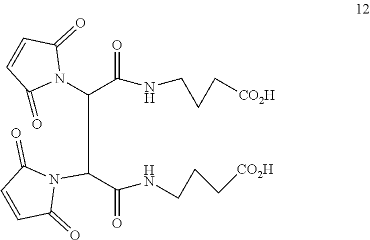 [SMILES: O=C(O)CCCNC(=O)C(C(C(=O)NCCCC(=O)O)N1C(=O)C=CC1=O)N1C(=O)C=CC1=O]

Compound 11 (1.6 g, 2.7 mmol) was dissolved in dichloromethane (10 mL) and trifluoroacetic acid (10 mL), and stirred at r.t. for 2 hours. The reaction was concentrated and then co-evaporated with dichloromethane twice, the residue was triturated with dichloromethane and ethyl acetate (10 mL/10 mL), to afford a white solid (1.3 g, 100% yield). MS-ESI (m/z): [M+H]⁺ calcd for C₂₀H₂₃N₄O₁₀, 479.13; found, 479.13.

### Example 19. Synthesis of dibenzyl ((3R,4S)-2,5-dioxotetrahydrofuran-3,4-diyl)-dicarbamate (13)

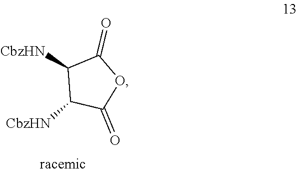 [SMILES: O=C(N[C@H]1C(=O)OC(=O)[C@@H]1NC(=O)OCc1ccccc1)OCc1ccccc1]

The solution of meso-2,3-bis(((benzyloxy)carbonyl)amino)succinic acid (100 g, 0.24 mol) in Ac₂O (1 L) was heated at 90° C. for 6 h, cooled and concentrated to dryness. The residue was co-evaporated with tolune and then triturated with a mixture of acetone (200 mL) and petroleum ether (400 mL). A white solid (80 g diastereomeric mixture) was collected and stirred with dichloromethane (300 mL) overnight. The racemic mixture (61 g) as a white solid was collected, and a racemic/meso mixture was also recovered from the mother liquid, which could be re-processed to afford a clean meso compound (2 g) and other racemic/meso mixture (15 g).

### Example 20. Synthesis of di-tert-butyl 4,4′-(((2R,3R)-2,3-bis(((benzyloxy)carbonyl)amino)succinyl)bis(azanediyl))dibutyrate (14)

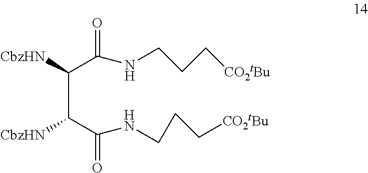 [SMILES: CC(C)(C)OC(=O)CCCNC(=O)[C@H](NC(=O)OCc1ccccc1)[C@@H](NC(=O)OCc1ccccc1)C(=O)NCCCC(=O)OC(C)(C)C]

To a solution of compound 13 (60 g, 0.151 mol) dissolved in tetrahydrofuran (1 L) was added tert-butyl aminobutyrate hydrochloride (31 g, 0.151 mol). The solution was cooled to 0° C. and triethylamine (42 mL, 0.302 mmol) was added. The reaction was warmed to r.t. and stirred for 30 minutes. Another portion of tert-butyl aminobutyrate (31 g, 0.151 mol), HATU (86.12 g, 0.226 mol) and triethylamine (42 mL, 0.302 mmol) were added, and the reaction was stirred at r.t. overnight, diluted with water (2 L), stirred for 10 minutes, and filtered to give a white solid (93 g, 88.13% yield). MS-ESI (m/z): [M+H]⁺ calcd for C₃₆H₅₁N₄O₁₀, 699.35; found, 699.35.

### Example 21. Synthesis of di-tert-butyl 4,4′-(((2R,3R)-2,3-diaminosuccinyl)bis(azanediyl)) dibutyrate (15)

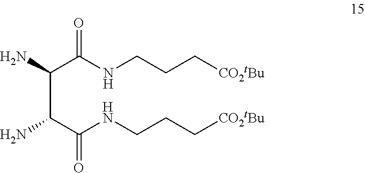 [SMILES: CC(C)(C)OC(=O)CCCNC(=O)[C@H](N)[C@@H](N)C(=O)NCCCC(=O)OC(C)(C)C]

Compound 14 (93 g, 0.133 mol) was dissolved in methanol (2 L), and then Pd/C (10 g) was added. The reaction flask was evacuated and back-filled with hydrogen, heated to 60° C., and stirred for 6 h. Filtration and concentration gave product 7 (57 g, 100% yield). MS-ESI (m/z): [M+H]⁺ calcd for C₂₀H₃₉N₄O₆, 431.28, found, 431.28.

### Example 22. Synthesis of Compound 16

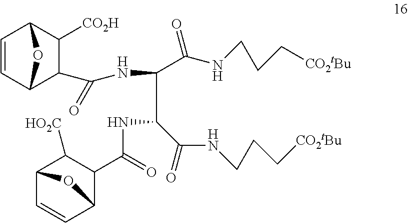 [SMILES: CC(C)(C)OC(=O)CCCNC(=O)[C@H](NC(=O)C1C(C(=O)O)[C@H]2C=C[C@@H]1O2)[C@@H](NC(=O)C1C(C(=O)O)[C@H]2C=C[C@@H]1O2)C(=O)NCCCC(=O)OC(C)(C)C]

Compound 15 (57 g, 0.13 mol) was dissolved in dichloromethane (600 mL), and exo-3,6-epoxy-1,2,3,6-tetrahydrophthalic anhydride (46 g, 0.27 mol) and triethylamine (36 ml, 0.27 mol) were added. The reaction was stirred at r.t. for 3 hours, concentrated to dryness, and co-evaporated with dichloromethane to give a white foamy solid (100 g, 100% yield). MS-ESI (m/z): [M+H]⁺ calcd for C₃₆H₅₁N₄O₁₄, 763.33; found, 763.33.

### Example 23. Synthesis of di-tert-butyl 4,4′-(((2R,3R)-2,3-bis((4R,7S)-1,3-dioxo-1,3,3a,4,7,7a-hexahydro-2H-4,7-epoxyisoindol-2-yl)succinyl)bis(azanediyl))dibutyrate (17)

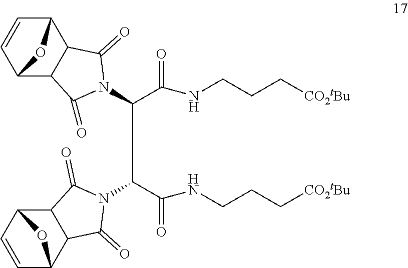 [SMILES: CC(C)(C)OC(=O)CCCNC(=O)[C@@H]([C@H](C(=O)NCCCC(=O)OC(C)(C)C)N1C(=O)C2C(C1=O)[C@H]1C=C[C@@H]2O1)N1C(=O)C2C(C1=O)[C@H]1C=C[C@@H]2O1]

To a solution of compound 16 (100 g, 0.13 mol) dissolved in DMF (1 L), were added EDC (75 g, 0.39 mol), HOBt (53 g, 0.39 mol), and DBU (59 g, 0.39 mol) slowly. The mixture was heated to 60° C. and stirred for 5 hours, cooled to r.t. and poured into water (2 L), extracted with dichloromethane (3×500 mL). The combined organic phases were washed with 2 N HCl (300 mL), brine (300 mL), dried over sodium sulfate, filtered and concentrated. The residue was triturated with petroleum ether/ethyl acetate (200 mL/500 mL), and the white solid was filtered off. The filtrate was concentrated and purified by a silica gel column to give a white foamy solid (67 g, 70% yield). MS-ESI (m/z): [M+H]⁺ calcd for C₃₆H₄₇N₄O₁₂, 727.31; found, 727.31.

### Example 24. Synthesis of di-tert-butyl 4,4′-(((2R,3R)-2,3-bis(2,5-dioxo-2,5-dihydro-1H-pyrrol-1-yl)succinyl)bis(azanediyl))dibutyrate (18)

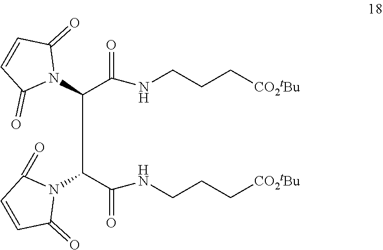 [SMILES: CC(C)(C)OC(=O)CCCNC(=O)[C@@H]([C@H](C(=O)NCCCC(=O)OC(C)(C)C)N1C(=O)C=CC1=O)N1C(=O)C=CC1=O]

Compound 17 (67 g, 92 mmol) was dissolved in a mixture of toluene (600 mL) and DMF (60 mL), heated to 120° C. and stirred under reflux for 4 hours. The reaction was then cooled to r.t. and stirred for more than 30 minutes, and a white solid precipitated, which was then collected by filtration and dried to give the desired product (41 g, 75% yield). MS-ESI (m/z): [M+H]⁺ calcd for C₂₈H₃₉N₄O₁₀, 591.26; found, 591.26.

### Example 25. Synthesis of 4,4′-(((2R,3R)-2,3-bis(2,5-dioxo-2,5-dihydro-1H-pyrrol-1-yl)succinyl)bis(azanediyl))dibutyric acid (19)

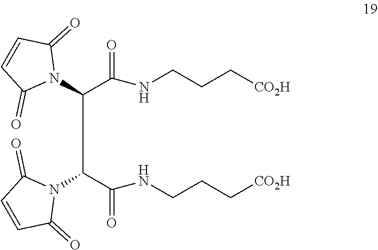 [SMILES: O=C(O)CCCNC(=O)[C@@H]([C@H](C(=O)NCCCC(=O)O)N1C(=O)C=CC1=O)N1C(=O)C=CC1=O]

Compound 18 (41 g, 69 mmol) was dissolved in dichloromethane (200 mL) and trifluoroacetic acid (200 mL), and stirred at r.t. for 2 hours. The reaction was concentrated and then co-evaporated with dichloromethane twice, the residue was triturated with dichloromethane and ethyl acetate (200 mL/200 mL), to afford a white solid (33 g, 99% yield).

### Example 26. Synthesis of tert-butyl (S)-(37-(((benzyloxy)carbonyl)amino)-31-oxo-2,5,8,11,14,17,20,23,26,29-decaoxa-32-azaoctatriacontan-38-oyl)glycinate (21)

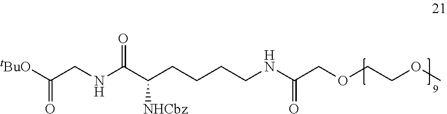

To a solution of (S)-37-(((benzyloxy)carbonyl)amino)-31-oxo-2,5,8,11,14,17,20, 23,26,29-decaoxa-32-azaoctatriacontan-38-oic acid (20, 4.0 g, 5.34 mmol) and H-Gly-OtBu·HCl (0.9 g, 5.34 mmol) in THF (40 mL), HATU (3.05 g, 8.01 mmol) and diisopropylethylamine (1.5 mL, 10.68 mmol) were added. The reaction was stirred at r.t. until completion, as indicated by LC-MS. The solvent was removed and the residue was poured into water (100 mL), extracted with dichloromethane (3×50 mL). The combined organic phases were washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give compound 21 (4.1 g, 89% yield). MS-ESI (m/z): [M+H]⁺ calcd for C₄₁H₇₂N₃O₁₆, 862.48; found, 862.48.

### Example 27. Synthesis of tert-butyl (S)-(37-amino-31-oxo-2,5,8,11,14,17,20,23,26,29-decaoxa-32-azaoctatriacontan-38-oyl)glycinate (22)

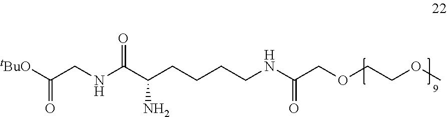

Compound 21 (2.9 g, 3.3 mmol) was dissolved in THF (50 mL), 10% palladium on carbon (0.3 g) was added, and the reaction flask was evacuated and back-filled with hydrogen for three times. After stirring for 2 hours, the reaction mixture was filtered and the filtrate was concentrated to give the title compound 2 (2.1 g, 84% yield). MS-ESI (m/z): [M+H]⁺ calcd for C₃₃H₆₆N₃O₁₄, 728.45; found 728.45.

### Example 28. Synthesis of di-tert-butyl (5S,13S,14S,22S)-13,14-bis(2,5-dioxo-2,5-dihydro-1H-pyrrol-1-yl)-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioate (23)

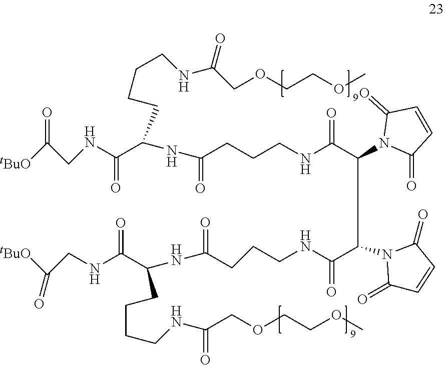

To a solution of compound 19 (120 mg, 0.251 mmol) and compound 22 (365 mg, 0.502 mmol) in mixed solvents of THF (10 mL) and DMF (5 mL), HATU (286 mg, 0.75 mmol) and diisopropylethylamine (82 μL, 0.5 mmol) were added. The reaction was stirred at r.t. until completion, as indicated by LC-MS. The solvent was removed and the residue was dissolved in dichloromethane (100 mL), washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give compound 23 (0.3 g, 62% yield). MS-ESI (m/z): [M+H]⁺ calcd for C₈₆H₁₄₉N₁₀O₃₆, 1898.01; found, 1898.01.

### Example 29. Synthesis of (5S,13S,14S,22S)-13,14-bis(2,5-dioxo-2,5-dihydro-1H-pyrrol-1-yl)-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioic acid (24)

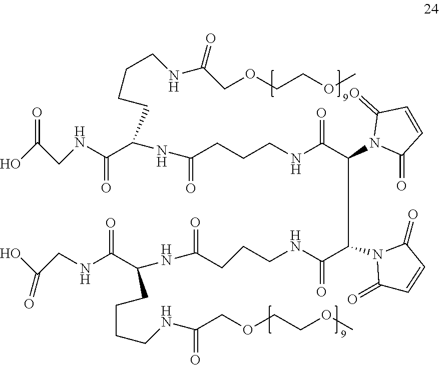

Compound 23 (0.3 g, 0.158 mmol) was dissolved in formic acid (10 mL) and dichloromethane (5 mL), and then heated to 60° C. The reaction was stirred until completion, as indicated by LC-MS and then concentrated to give the title compound (280 mg, 100% yield). MS-ESI (m/z): [M+H]⁺ calcd for C₇₈H₁₃₃N₁₀O₃₆, 1785.88; found 1785.88.

### Example 30. Synthesis of (2S,3S)-2,3-bis(2,5-dioxo-2,5-dihydro-1H-pyrrol-1-yl)-N1,N4-bis((S)-37-((2-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-2-oxoethyl)carbamoyl)-31,39-dioxo-2,5,8,11,14,17,20,23,26,29-decaoxa-32,38-diazadotetracontan-42-yl)succinamide (25)

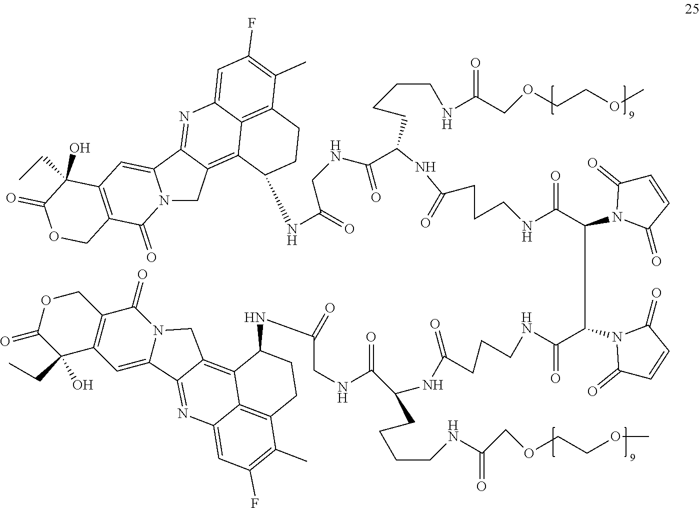

Compound 24 (200 mg, 0.102 mmol) and exatecan mesylate (108 mg 0.204 mmol) were dissolved in DMF (5 mL), HATU (116 mg, 0.306 mmol) and diisopropylethylamine (71 μL, 0.408 mmol) were added. The reaction was stirred at r.t. until complete conversion, and then concentrated and purified by preparative HPLC to give product 25 (120 mg, 46% yield). MS-ESI (m/z): [M+H]⁺ calcd for C₁₂₆H₁₇₃F₂N₁₆O₄₂, 2620.18; found, 2620.18.

### Example 31. Synthesis of tert-butyl (S)-(S)-(37-(((benzyloxy)carbonyl)amino)-31-oxo-2,5,8,11,14,17,20,23,26,29-decaoxa-32-azaoctatriacontan-38-oyl)glycylglycinate (26)

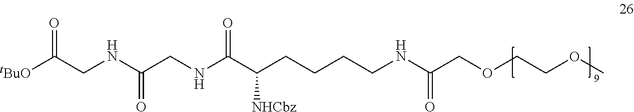

To a solution of compound 20 (5 g, 6.78 mmol) and H-Gly-Gly-O^{t}Bu·HCl (1.5 g, 6.78 mmol) in THF (50 mL), HATU (3.81 g, 10.01 mmol) and diisopropylethylamine (1.8 ml, 13.35 mmol) were added. The reaction was stirred at r.t. until completion, as indicated by LC-MS. The solvent was removed and the residue was poured into water (100 mL), extracted with dichloromethane (3×50 mL). The combined organic phases were washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give compound 26 (3.7 g, 60% yield). MS-ESI (m/z): [M+H]⁺ calcd for C₄₃H₇₅N₄O₁₇, 919.50; found, 919.50.

### Example 32. Synthesis of tert-butyl (S)-(S)-(37-amino-31-oxo-2,5,8,11,14,17,20,23,26,29-decaoxa-32-azaoctatriacontan-38-oyl)glycylglycinate (27)

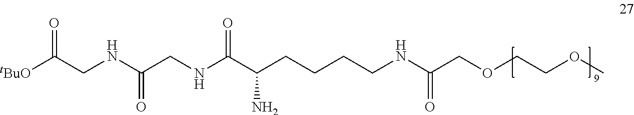

Compound 26 (819 mg, 0.89 mmol) was dissolved in THF (20 mL), 10% palladium on carbon (0.1 g) was added, and the reaction flask was evacuated and back-filled with hydrogen for three times. After stirring for 2 hours, the reaction mixture was filtered and the filtrate was concentrated to give the title compound 27 (700 mg, 100% yield). MS-ESI (m/z): [M+H]⁺ calcd for C₃₅H₆₉N₄O₁₅, 785.47; found, 785.47.

### Example 33. Synthesis of di-tert-butyl (8S,16S,17S,25S)-16,17-bis(2,5-dioxo-2,5-dihydro-1H-pyrrol-1-yl)-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioate (28)

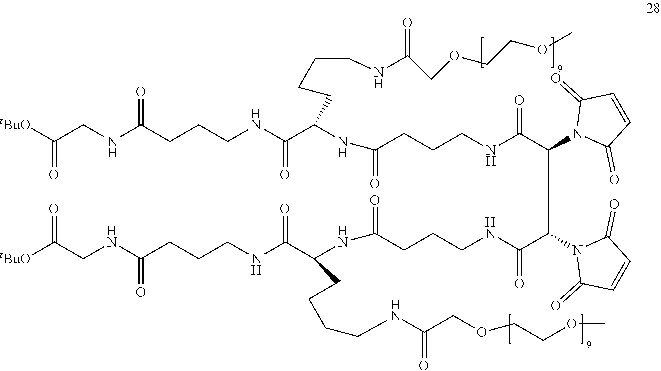

To a solution of compound 19 (180 mg, 0.376 mmol) and compound 27 (649 mg, 0.828 mmol) in mixed solvents of THF (10 mL) and DMF (5 mL), HATU (429 mg, 1.13 mmol) and diisopropylethylamine (186 μL, 1.13 mmol) were added. The reaction was stirred at r.t. until completion, as indicated by LC-MS. The solvent was removed and the residue was dissolved in dichloromethane (100 mL), washed with water (30 mL), saturated sodium bicarbonate (30 mL), 2 N HCl (30 mL), and brine (30 mL), dried over sodium sulfate, filtered and concentrated, purified by silica gel column (dichloromethane/MeOH=100/0 to 20/1 to 10/1), to give compound (0.45 g, 59% yield). MS-ESI (m/z): [M+H]⁺ calcd for C₉₀H₁₅₅N₁₂O₃₈, 2012.05; found, 2012.05.

### Example 34. Synthesis of (8S,16S,17S,25S)-16,17-bis(2,5-dioxo-2,5-dihydro-1H-pyrrol-1-yl)-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioic acid (29)

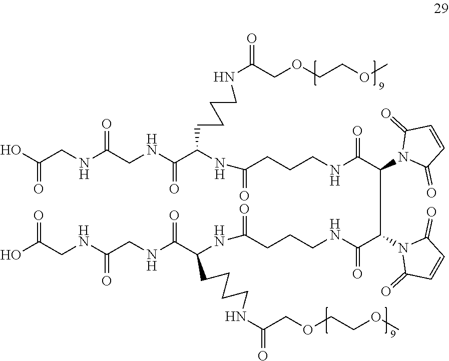

Compound 28 (0.30 g, 0.194 mmol) was dissolved in formic acid (10 mL) and dichloromethane (5 mL), and then heated to 60° C. The reaction was stirred until completion, as indicated by LC-MS and then concentrated to give the title compound (280 mg, 100% yield). MS-ESI (m/z): [M+H]⁺ calcd for C₈₂H₁₃₉N₁₂O₃₈, 1899.92; found, 1899.92.

### Example 35 Synthesis of (2S,3S)-2,3-bis(2,5-dioxo-2,5-dihydro-1H-pyrrol-1-yl)-N1,N4-bis((S)-37-((2-((2-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-2-oxoethyl)amino)-2-oxoethyl)carbamoyl)-31,39-dioxo-2,5,8,11,14,17,20,23,26,29-decaoxa-32,38-diazadotetracontan-42-yl)succinamide (30)

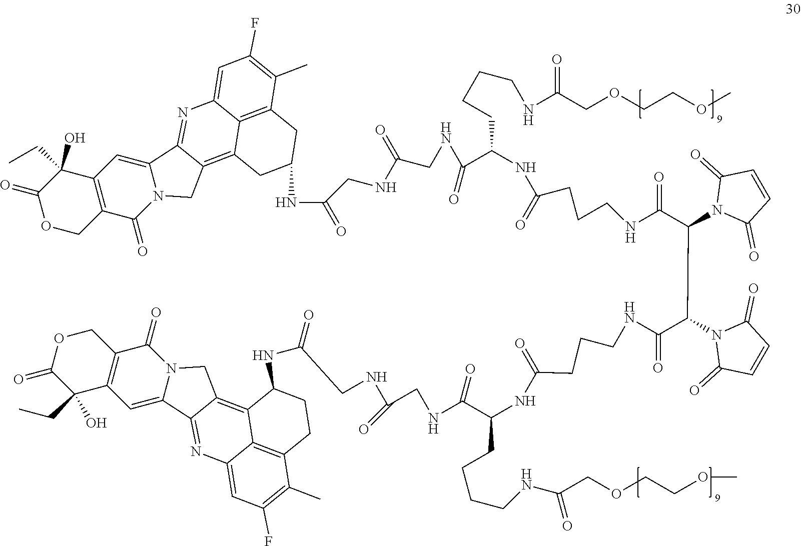

Compound 29 (142 mg, 0.075 mmol) and exatecan mesylate (65 mg, 0.15 mmol) were dissolved in DMF (5 mL), HATU (85 mg, 0.225 mmol) and diisopropylethylamine (37 μL, 0.225 mmol) were added. The reaction was stirred at r.t. until complete conversion, and then concentrated and purified by preparative HPLC to give the title compound (60 mg, 28% yield). MS-ESI (m/z): [M+H]⁺ calcd for C₁₃₀H₁₇₉F₂N₁₈O₄, 2734.22; found, 2734.22.

### Example 36. Synthesis of tert-butyl (S)-(37-(((benzyloxy)carbonyl)amino)-31,38-dioxo-2,5,8,11,14,17,20,23,26,29-decaoxa-32,39-diazatritetracontan-43-oyl)glycinate (32)

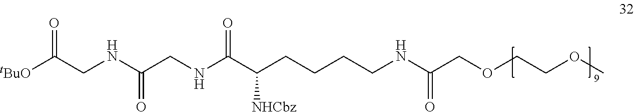

To a solution of compound 20 (5.00 g, 6.78 mmol) and tert-butyl (4-aminobutanoyl)glycinate hydrochloride (31, 1.71 g, 6.78 mmol) in THF (50 mL), HATU (3.81 g, 10.01 mmol) and diisopropylethylamine (1.8 mL, 13.35 mmol) were added. The reaction was stirred at r.t. until completion, as indicated by LC-MS. The solvent was removed and the residue was poured into water (100 mL), extracted with dichloromethane (3×50 mL). The combined organic phases were washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give compound 32 (5.4 g, 85% yield). MS-ESI (m/z): [M+H]⁺ calcd for C₄₅H₇₈N₄O₁₇, 947.54; found, 947.57.

### Example 37. Synthesis of tert-butyl (S)-(37-amino-31,38-dioxo-2,5,8,11,14,17,20,23,26,29-decaoxa-32,39-diazatritetracontan-43-oyl)glycinate (33)

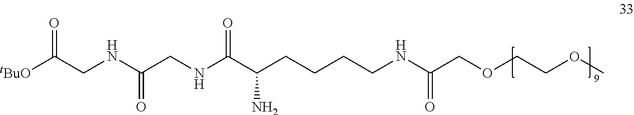

Compound 32 (819 mg, 0.86 mmol) was dissolved in THF (20 mL), 10% palladium on carbon (0.1 g) was added, and the reaction flask was evacuated and back-filled with hydrogen for three times. After stirring for 2 hours, the reaction mixture was filtered and the filtrate was concentrated to give the title compound (700 mg, 100% yield). MS-ESI (m/z): [M+H]⁺ calcd for C₃₇H₇₃N₄O₁₅, 813.50; found, 813.50.

### Example 38. Synthesis of di-tert-butyl (10S,18S,19S,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3,8,11,16,21,26,29,34-octaazahexatriacontanedioate (34)

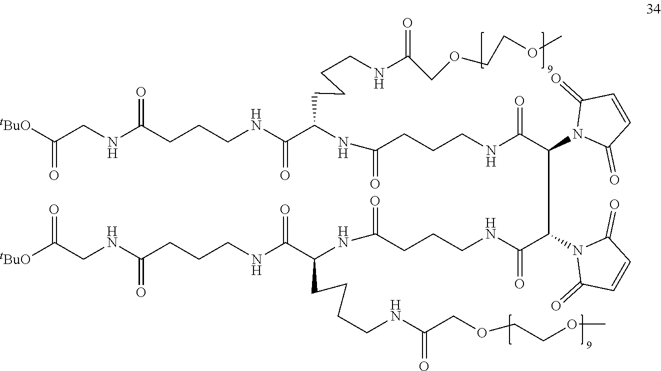

To a solution of compound 19 (246 mg, 0.514 mmol) and compound 33 (1.0 g, 1.286 mmol) in mixed solvents of THF (10 mL) and DMF (5 mL), HATU (586 mg, 1.54 mmol) and diisopropylethylamine (245 μL, 1.54 mmol) were added. The reaction was stirred at r.t. until completion, as indicated by LC-MS. The solvent was removed and the residue was dissolved in dichloromethane (100 mL), washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give the title compound (0.54 g, 51% yield). MS-ESI (m/z): [M+H]⁺ calcd for C₉₄H₁₆₃N₁₂O₃₈, 2068.11; found, 2068.11.

### Example 39. Synthesis of (10S,18S,19S,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3,8,11,16,21,26,29,34-octaazahexatriacontanedioic acid (35)

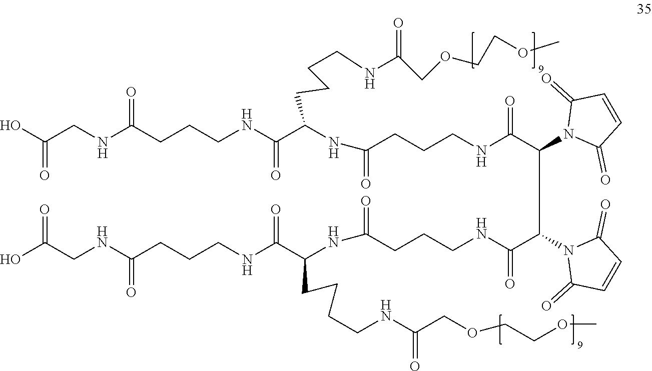

Compound 34 (0.50 g, 0.242 mmol) was dissolved in formic acid (10 mL) and dichloromethane (5 mL), and then heated to 60° C. The reaction was stirred until completion, as indicated by LC-MS and then concentrated to give the title compound (470 mg, 100% yield). MS-ESI (m/z): [M+H]⁺ calcd for C₈₆H₁₄₇N₁₂O₃₈, 1955.99; found, 1955.99.

### Example 40. Synthesis of (2S,3S)-2,3-bis(2,5-dioxo-2,5-dihydro-1H-pyrrol-1-yl)-N1,N4-bis((S)-37-((4-((2-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-2-oxoethyl)amino)-4-oxobutyl)carbamoyl)-31,39-dioxo-2,5,8,11,14,17,20,23,26,29-decaoxa-32,38-diazadotetracontan-42-yl)succinamide (36)

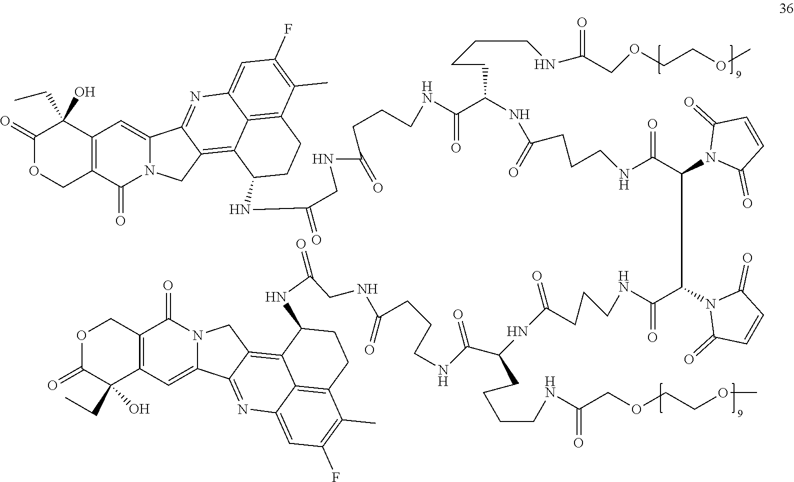

Compound 35 (200 mg, 0.102 mmol) and exatecan mesylate (108 mg, 0.204 mmol) were dissolved in DMF (5 mL), HATU (116 mg, 0.306 mmol) and diisopropylethylamine (53 μL, 0.306 mmol) were added. The reaction was stirred at r.t. until complete conversion, and then concentrated and purified by preparative HPLC to give the title compound (120 mg, 42% yield). MS-ESI (m/z): [M+H]⁺ calcd for C₁₃₀H₁₇₉F₂N₁₈O₄, 2734.22; found, 2734.22.

### Example 41. Synthesis of tert-butyl ((benzyloxy)carbonyl)glycylglycylglycinate (38)

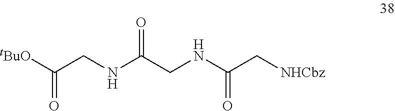 [SMILES: CC(C)(C)OC(=O)CNC(=O)CNC(=O)CNC(=O)OCc1ccccc1]

Cbz-Gly-Gly-OH (20.3 g, 76.2 mmol) and H-Gly-O^{t}Bu (10.0 g, 76.2 mmol) were dissolved in dichloromethane (300 mL) and cooled to 0° C. in an ice-water bath. After addition of HATU (34.8 g, 91.5 mmol) and triethylamine (32 mL, 228.7 mmol), the reaction was warmed to r.t. and stirred overnight. The reaction solution was washed with brine, dried, concentrated, and purified by column chromatography (dichloromethane:MeOH=20:1) to give 25 g of the desired product with a yield of 86%. MS-ESI (m/z): [M+H]⁺ calcd for C₁₈H₂₅N₃O₆, 380.17; found, 379.9.

### Example 42. Synthesis of tert-butyl glycylglycylglycinate (39)

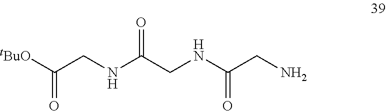 [SMILES: CC(C)(C)OC(=O)CNC(=O)CNC(=O)CN]

Compound 38 (16.5 g, 43.5 mmol) was dissolved in THF (300 mL), 10% palladium on carbon (2.0 g) was added, and the reaction flask was evacuated and back-filled with hydrogen for three times. After stirring for 4 hours, the reaction mixture was filtered and the filtrate was concentrated to give an off-white solid (10.2 g, 95% yield). MS-ESI (m/z): [M+H]⁺ calcd for C₁₀H₁₉N₃O₄, 246.14; found, 246.14.

### Example 43. Synthesis of tert-butyl (S)-(S)-(S)-(37-(((benzyloxy)carbonyl)amino)-31-oxo-2,5,8,11,14,17,20,23,26,29-decaoxa-32-azaoctatriacontan-38-oyl)glycylglycylglycinate (40)

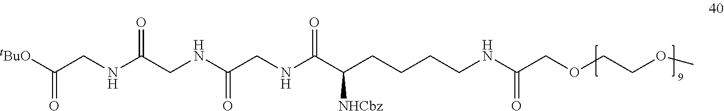

To a solution of compound 20 (5.0 g, 6.78 mmol) and compound 39 (1.67 g, 6.78 mmol) in THF (50 mL), HATU (3.81 g, 10.01 mmol) and diisopropylethylamine (1.8 ml, 13.35 mmol) were added. The reaction was stirred at r.t. until completion, as indicated by LC-MS. The solvent was removed and the residue was poured into water (100 mL), extracted with dichloromethane (3×50 mL). The combined organic phases were washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give compound 40 (4.3 g, 65% yield). MS-ESI (m/z): [M+H]⁺ calcd for C₄₇H₇₇N₅O₁₈, 976.53; found 976.53.

### Example 44. Synthesis of tert-butyl (S)-(S)-(S)-(37-amino-31-oxo-2,5,8,11,14,17,20,23,26,29-decaoxa-32-azaoctatriacontan-38-oyl)glycylglycylglycinate (41)

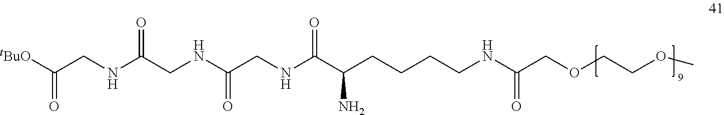

Compound 40 (1.5 g, 1.53 mmol) was dissolved in THF (20 mL), 10% palladium on carbon (0.1 g) was added, and the reaction flask was evacuated and back-filled with hydrogen for three times. After stirring for 2 hours, the reaction mixture was filtered and the filtrate was concentrated to give the title compound 41 (1.3 g, 100% yield). MS-ESI (m/z): [M+H]⁺ calcd for C₃₇H₇₁N₅O₁₆, 842.49; found, 842.49.

### Example 45. Synthesis of di-tert-butyl (11S,19S,20S,28S)-19,20-bis(2,5-dioxo-2,5-dihydro-1H-pyrrol-1-yl)-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioate (42)

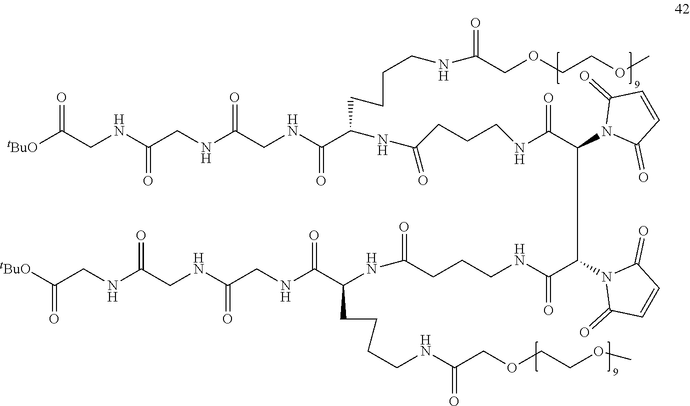

Compound 20 (911 mg, 1.08 mmol) and compound 41 (225 mg, 0.470 mmol) were dissolved in DMF (5 mL), HATU (536 mg, 1.411 mmol) and diisopropylethylamine (0.233 mL, 1.411 mmol) were added. The reaction mixture was stirred for 30 minutes, and then poured into 50 mL of water, extracted with 40 mL of dichloromethane for 3 times. The organic phase was washed with 20 mL of brine, dried over anhydrous sodium sulfate, filtered and concentrated. The residue was purified by silica gel column to give compound 42 as an oil (671 mg, 0.316 mmol, 67%). MS-ESI (m/z): [M+H]⁺ calcd for C₉₄H₁₆₀N₁₄O₄₀, 2127.37; found, 2128.16.

### Example 46. Synthesis of (11S,19S,20S,28S)-19,20-bis(2,5-dioxo-2,5-dihydro-1H-pyrrol-1-yl)-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioic acid (43)

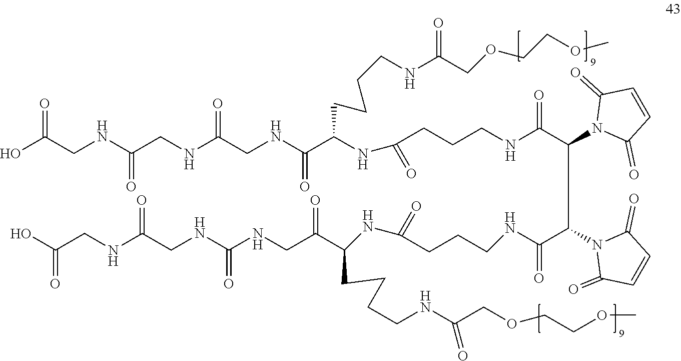

Compound 42 (671 mg, 0.316 mmol) was dissolved in dichloromethane (4 mL) and treated with trifluoroacetic acid (2 mL). After stirring for 8 hours, the reaction solution was concentrated, to give compound 43 as an oil (456 mg, 0.226 mmol, 71%). MS-ESI (m/z): [M+H]⁺ calcd for C₈₆H₁₄₄N₁₄O₄₀, 2015.16; found, 2016.09.

### Example 47. Synthesis of bis(perfluorophenyl) (11S,19S,20S,28S)-19,20-bis(2,5-dioxo-2,5-dihydro-1H-pyrrol-1-yl)-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioate (44)

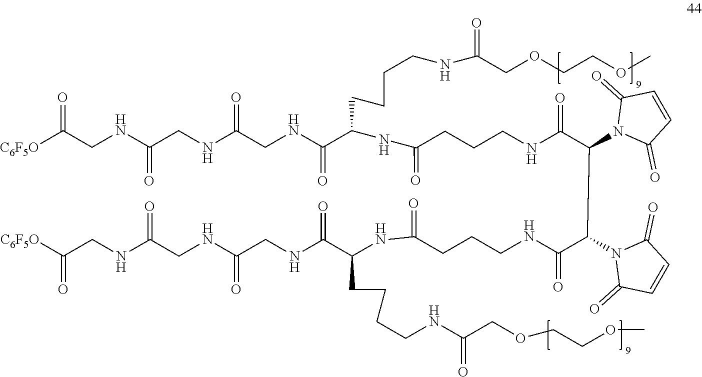

Compound 43 (323 mg, 0.160 mmol) was dissolved in dichloromethane (10 mL), and pentafluorophenol (73.8 mg, 0.401 mmol) and EDCI (76.8 mg, 0.401 mmol) were added. After stirring for 3 hours, the reaction solution was washed with 10 mL of brine, dried over anhydrous sodium sulfate, filtered and concentrated to give compound 44 as an oil (376 mg, 0.160 mmol, 99%). MS-ESI (m/z): [M+H]⁺ calcd for C₉₈H₁₄₂F₁₀N₁₄O₄₀, 2347.26; found, 2348.26.

### Example 48. Synthesis of (2S,3S)-2,3-bis(2,5-dioxo-2,5-dihydro-1H-pyrrol-1-yl)-N1,N4-bis((S)-37-((2-((2-((2-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-2-oxoethyl)amino)-2-oxoethyl)amino)-2-oxoethyl)carbamoyl)-31,39-dioxo-2,5,8,11,14,17,20,23,26,29-decaoxa-32,38-diazadotetracontan-42-yl)succinamide (45)

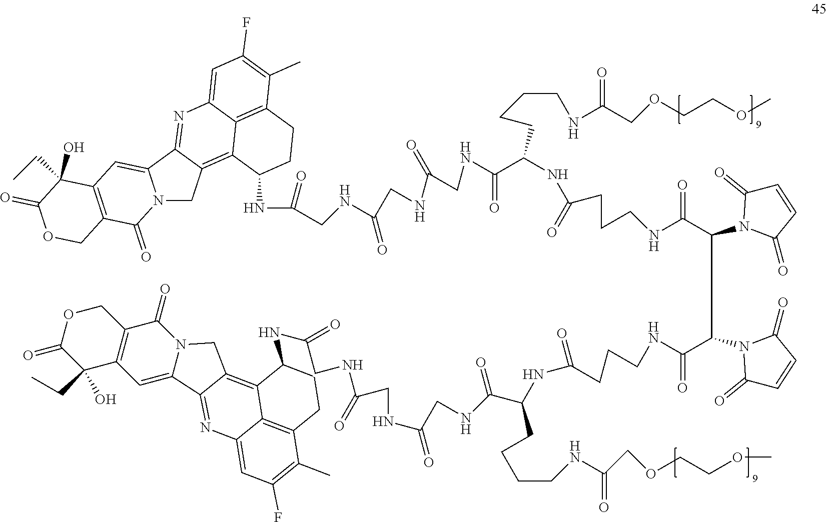

Compound 44 (188 mg, 0.080 mmol) and exatecan mesylate (87 mg, 0.200 mmol) were dissolved in DMF (2 mL), and diisopropylethylamine (0.053 mL, 0.321 mmol) was added. The reaction was stirred for 1 hour, and purified by preparative HPLC (acetonitrile/water) to give compound 45 as a solid (48 mg, 0.017 mmol, 21%). MS-ESI (m/z): [M+H]⁺ calcd for C₁₃₄H₁₈₄F₂N₂₀O₄₆, 2850.04; found, 1426.00.

### Example 49. Synthesis of (S)-(S)-(37-(((benzyloxy)carbonyl)amino)-31-oxo-2,5,8,11,14,17,20,23,26,29-decaoxa-32-azaoctatriacontan-38-oyl)glycylglycine (46)

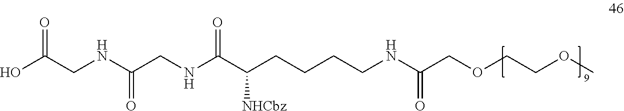

Compound 26 (12.0 g, 13.06 mmol) was dissolved in dichloromethane (20 mL) and formic acid (40 mL). After stirring for 3 hours, the reaction solution was concentrated, diluted with dichloromethane, washed with water twice, and brine once. The solution was dried over anhydrous sodium sulfate, filtered and concentrated to give compound 46 (10.4 g, 920 yield).

### Example 50. Synthesis of tert-butyl ((S)-37-(((benzyloxy)carbonyl)amino)-31-oxo-2,5,8,11,14,17,20,23,26,29-decaoxa-32-azaoctatriacontan-38-oyl)glycylglycyl-L-alaninate (47)

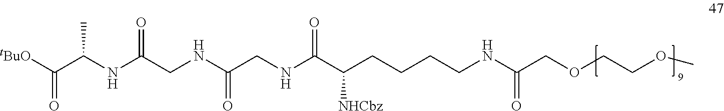

To a solution of compound 46 (2.10 g, 2.43 mmol) in 30 mL of dichloromethane, H-Ala-O^{t}Bu (0.53 g, 2.92 mmol), HATU (1.41 g, 3.65 mmol) and diisopropylethylamine (0.63 g, 4.87 mmol) were added in sequence, and the reaction was carried out at r.t. for 30 min. Water was added to the reaction solution and the layers were separated. The organic phase was washed with 0.5 N HCl, 0.5 N NaHCO₃ and brine, dried over anhydrous sodium sulfate, filtered, and the filtrate was concentrated. The residue was purified by preparative HPLC (water/acetonitrile), and the proper fractions were concentrated to give the title compound (1.4 g, 58% yield). MS-ESI (m/z): [M+H]⁺ calcd for C₄₆H₇₉N₅O₁₈, 991.54; found, 991.20.

### Example 51. Synthesis of tert-butyl ((S)-37-amino-31-oxo-2,5,8,11,14,17,20,23,26,29-decaoxa-32-azaoctatriacontan-38-oyl)glycylglycyl-L-alaninate (48)

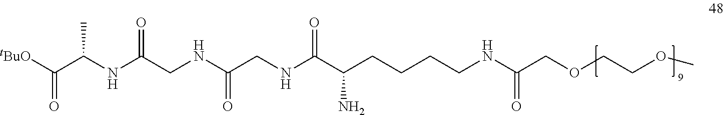

Compound 47 (1.4 g, 1.40 mmol) was dissolved in THF (20 mL), 10% palladium on carbon (0.1 g) was added, and the reaction flask was evacuated and back-filled with hydrogen for three times. After stirring for 2 hours, the reaction mixture was filtered and the filtrate was concentrated to give the title compound 48 (1.2 g, 99% yield). MS-ESI (m/z): [M+H]⁺ calcd for C₃₈H₇₃N₅O₁₆, 856.51; found, 857.00.

### Example 52. Synthesis of di-tert-butyl (2S,11S,19S,20S,28S,37S)-19,20-bis(2,5-dioxo-2,5-dihydro-1H-pyrrol-1-yl)-2,37-dimethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioate (49)

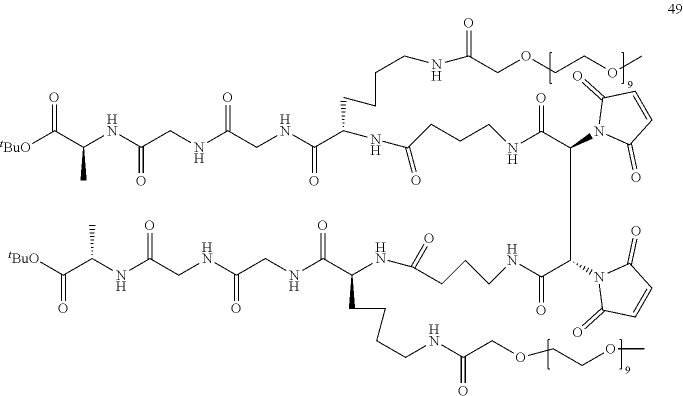

To a solution of compound 19 (0.31 g, 0.64 mmol) in 5 mL DMF was added compound 48 (1.2 g, 1.4 mmol) in 5 mL of DMF, followed by HATU (0.75 g, 1.93 mmol) and diisopropylethylamine (0.35 g, 2.57 mmol), and the reaction was stirred for 1 h, concentrated, dissolved in dichloromethane, washed with water, 0.5 N HCl, 0.5 NaHCO₃, and brine, dried over anhydrous sodium sulfate, filtered, and the filtrate was concentrated to yield the title compound (1.1 g, 79% yield). MS-ESI (m/z): [M+H]⁺ calcd for C₉₆H₁₆₄N₁₄O₄₀, 2154.12; found, 2155.40.

### Example 53. Synthesis of (2S,11S,19S,20S,28S,37S)-19,20-bis(2,5-dioxo-2,5-dihydro-1H-pyrrol-1-yl)-2,37-dimethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioic acid (50)

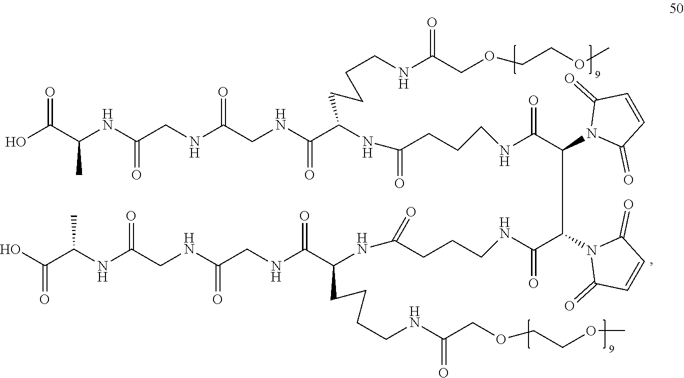

Compound 49 (1.1 g, 0.51 mmol) was dissolved in dichloromethane (10 mL) and formic acid (20 mL). After stirring at 50° C. for 2 hours, the reaction solution was concentrated, and the residue was purified by preparative HPLC (water/acetonitrile) to give compound 50 (300 mg, 30% yield). MS-ESI (m/z): [M+H]⁺ calcd for C₈₈H₁₄₈N₁₄O₄₀, 2042.00; found, 2043.2.

### Example 54. Synthesis of (2S,3S)-2,3-bis(2,5-dioxo-2,5-dihydro-1H-pyrrol-1-yl)-N1,N4-bis((S)-37-((2-((2-(((S)-1-(((1 S, 9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-1-oxopropan-2-yl)amino)-2-oxoethyl)amino)-2-oxoethyl)carbamoyl)-31,39-dioxo-2,5,8,11,14,17,20,23,26,29-decaoxa-32,38-diazadotetracontan-42-yl)succinamide (51)

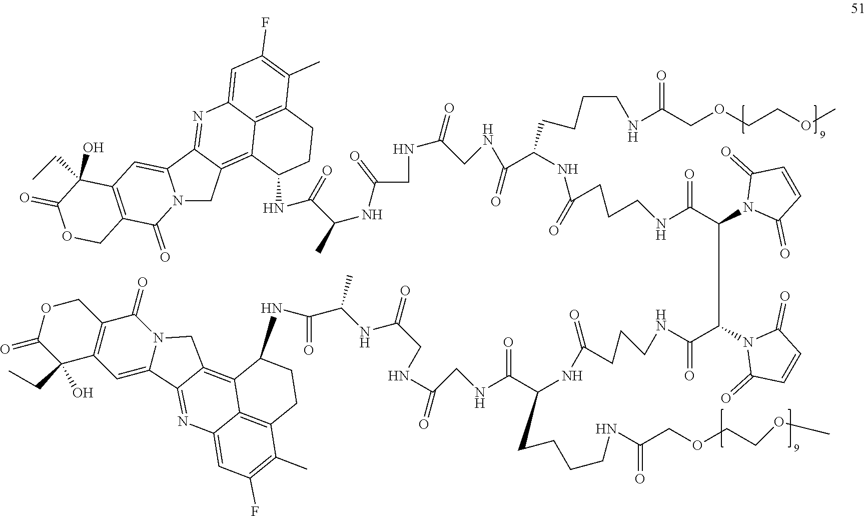

To a solution of compound 50 (70.0 mg, 0.034 mmol) in 1 mL of DMF, were added exatecan mesylate (32.0 mg, 0.075 mmol) and HATU (39.0 mg, 0.103 mmol), followed by diisopropylethylamine (18 mg, 0.137 mmol). The reaction was stirred at r.t. for 30 min, concentrated, and purified by preparative HPLC (water/acetonitrile). The fraction pool was combined, concentrated and lyophilized to yield the title compound (57.3 mg, yield 580%). MS-ESI (m/z): [M+H]⁺ calcd for C₁₃₆H₁₈₈F₂N₂₀O₄₆, 2876.30; found, 2878.10.

### Example 55. Synthesis of tert-butyl ((benzyloxy)carbonyl)glycylglycylglycylglycinate (52)

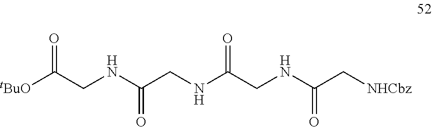 [SMILES: CC(C)(C)OC(=O)CNC(=O)CNC(=O)CNC(=O)CNC(=O)OCc1ccccc1]

To a solution of compound 39 (1.92 g, 7.83 mmol) in 50 mL of dichloromethane, H-Ala-O^{t}Bu (1.96 g, 9.41 mmol), HATU (3.56 g, 9.41 mmol) and diisopropylethylamine (1.22 g, 9.41 mmol) were added in sequence, and the reaction was stirred at r.t. for 30 min. and then quenched with water. After phase separation, the organic phase was washed with 0.5 N HCl, 0.5 N NaHCO₃ and brine, dried over anhydrous sodium sulfate, filtered, and the filtrate was concentrated. The residue was purified by column chromatography to give the title compound (2.74 g, 80% yield). MS-ESI (m/z): [M+H]⁺ calcd for C₂₀H₂₈N₄O₇, 437.20; found 437.20.

### Example 56. Synthesis of tert-butyl glycylglycylglycylglycinate (53)

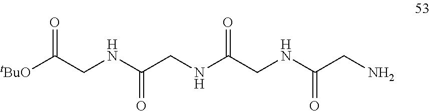 [SMILES: CC(C)(C)OC(=O)CNC(=O)CNC(=O)CNC(=O)CN]

Compound 52 (2.74 g, 6.28 mmol) was dissolved in THF (20 mL), 10% palladium on carbon (0.10 g) was added, and the reaction flask was evacuated and back-filled with hydrogen for three times. After stirring for 2 hours, the reaction mixture was filtered and the filtrate was concentrated to give the title compound 53 (1.90 g, 99% yield). MS-ESI (m/z): [M+H]⁺ calcd for C₁₂H₂₂N₄O₅, 303.16; found, 303.18.

### Example 57. Synthesis of tert-butyl (S)-(S)-(S)-(S)-(37-(((benzyloxy)carbonyl)amino)-31-oxo-2,5,8,11,14,17,20,23,26,29-decaoxa-32-azaoctatriacontan-38-oyl)glycylglycylglycylglycinate (54)

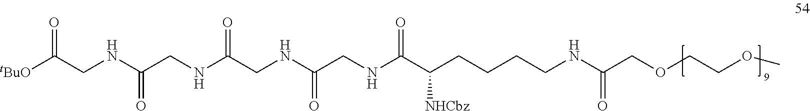

To a solution of compound 20 (4.5 g, 6.01 mmol) and H-Gly-Gly-Gly-Gly-OtBu (1.9 g, 6.28 mmol) in THF (20 mL) and DMF (20 mL), HATU (3.43 g, 9.01 mmol) and diisopropylethylamine (1.9 mL, 12.0 mmol) were added. The reaction was stirred at r.t. until completion, as indicated by LC-MS. The solvent was removed and the residue was poured into water (100 mL), extracted with dichloromethane (3×50 mL). The combined organic phases were washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give compound 54 (4.9 g, 79% yield). MS-ESI (m/z): [M+H]⁺ calcd for C₄₇H₈₁N₆O₁₉, 1033.55; found, 1033.55.

### Example 58. Synthesis of tert-butyl (S)-(S)-(S)-(S)-(37-amino-31-oxo-2,5,8,11,14,17,20,23,26,29-decaoxa-32-azaoctatriacontan-38-oyl)glycylglycylglycylglycinate (55)

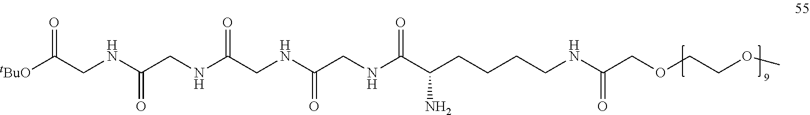

Compound 54 (4.9 g, 4.7 mmol) was dissolved in THF (80 mL), 10% palladium on carbon (0.5 g) was added, and the reaction flask was evacuated and back-filled with hydrogen for three times. After stirring for 2 hours, the reaction mixture was filtered and the filtrate was concentrated to give the title compound (700 mg, 100% yield). MS-ESI (m/z): [M+H]⁺ calcd for C₃₉H₇₅N₆O₁₇, 899.51; found, 899.51.

### Example 59. Synthesis of di-tert-butyl (14S,22S,23S,31S)-22,23-bis(2,5-dioxo-2,5-dihydro-1H-pyrrol-1-yl)-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioate (56)

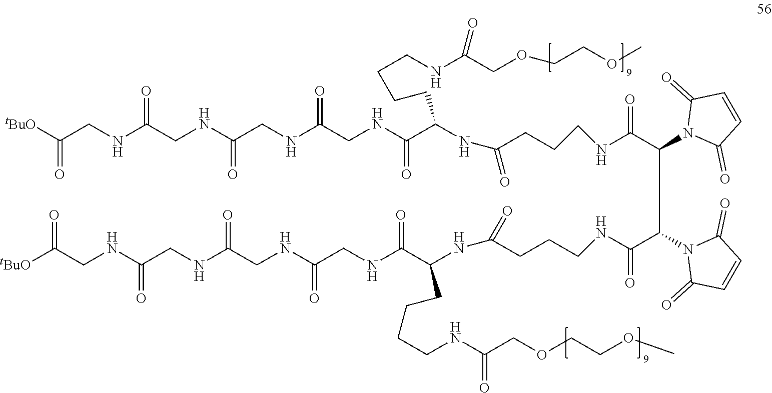

To a solution of compound 19 (1.0 g, 2.09 mmol) and compound 55 (3.7 g, 4.1 mmol) in a mixed solvent of THF (30 mL) and DMF (15 mL), HATU (2.38 mg, 6.27 mmol) and diisopropylethylamine (1.4 mL, 8.36 mmol) were added. The reaction was stirred at r.t. until completion, as indicated by LC-MS. The solvent was removed and the residue was dissolved in dichloromethane (300 mL), washed with water (50 mL), saturated sodium bicarbonate (50 mL), 2 N HCl (50 mL), and brine (50 mL), dried over sodium sulfate, filtered and concentrated, purified by silica gel column (dichloromethane/MeOH=100/0 to 20/1 to 10/1), to give the title compound (3.2 g, 68% yield). MS-ESI (m/z): [M+H]⁺ calcd for C₉₈H₁₆₇N₁₆O₄₂, 2240.13; found, 2240.13.

### Example 60. Synthesis of (14S,22S,23S,31S)-22,23-bis(2,5-dioxo-2,5-dihydro-1H-pyrrol-1-yl)-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioic acid (57)

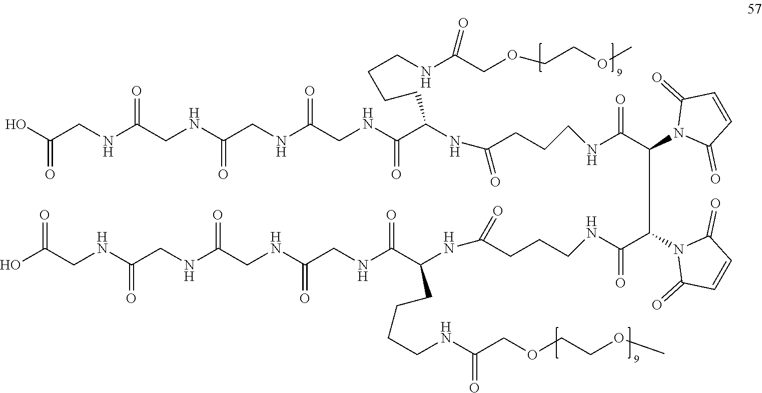

Compound 56 (3.2 g, 1.4 mmol) was dissolved in formic acid (20 mL) and dichloromethane (10 mL), and then heated to 60° C. The reaction was stirred until completion, as indicated by LC-MS and then concentrated to give the title compound (690 mg, 22% yield). MS-ESI (m/z): [M+H]⁺ calcd for C₉₀H₁₅₁N₁₆O₄₂, 2128.01; found, 2128.01.

### Example 61. Synthesis of (2S,3S)-2,3-bis(2,5-dioxo-2,5-dihydro-1H-pyrrol-1-yl)-N1,N4-bis((S)-37-((2-((2-((2-((2-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-2-oxoethyl)amino)-2-oxoethyl)amino)-2-oxoethyl)amino)-2-oxoethyl)carbamoyl)-31,39-dioxo-2,5,8,11,14,17,20,23,26,29-decaoxa-32,38-diazadotetracontan-42-yl)succinamide (58a)

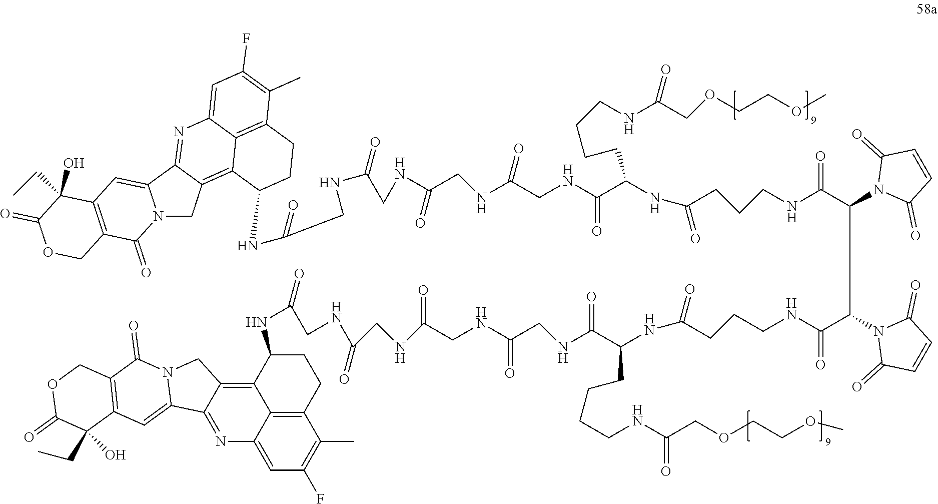

Compound 57 (420 mg, 0.197 mmol) and exatecan mesylate (209 mg 0.395 mmol) were dissolved in DMF (5 mL), HATU (255 mg, 0.592 mmol) and diisopropylethylamine (130 μL, 0.789 mmol) were added. The reaction was stirred at r.t. until complete conversion, and then concentrated and purified by preparative HPLC to give the title compound (274 mg, 47% yield). MS-ESI (m/z): [M+H]⁺ calcd for C₁₃₈H₁₉₁F₂N₂₂O₄₈, 2962.31; found, 2962.31.

### Example 62. Synthesis of tert-butyl ((benzyloxy)carbonyl)-L-alanyl-L-alaninate (59)

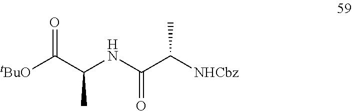 [SMILES: C[C@H](NC(=O)OCc1ccccc1)C(=O)N[C@@H](C)C(=O)OC(C)(C)C]

Cbz-Ala-OH (15.0 g, 67.1 mmol) and H-Ala-O^{t}Bu·HCl (12.3 g, 67.7 mmol) were dissolved in dichloromethane (100 mL), cooled to 0° C. and EDC (25.7 g, 134 mmol) was added, followed by diisopropylethylamine (18.0 g, 134 mmol) dropwise. After stirring for about 30 min, the reaction was washed with 100 mL of water, 100 mL of brine, dried over anhydrous sodium sulfate, filtered, concentrated, purified by a silica gel column, eluted with petroleum ether and ethyl acetate to give a colorless liquid (19.2 g, 81% yield). MS-ESI (m/z): [M+H]⁺ calcd for C₁₈H₂₇N₂O₅, 351.18; found, 351.18

### Example 63. Synthesis of ((benzyloxy)carbonyl)-L-alanyl-L-alanine (60)

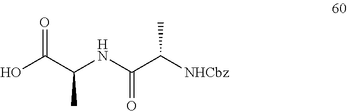 [SMILES: C[C@H](NC(=O)[C@H](C)NC(=O)OCc1ccccc1)C(=O)O]

Compound 59 (5.0 g, 14.0 mmol) was dissolved in dichloromethane (20 mL) and treated with 20 mL of trifluoroacetic acid at r.t. for 4 h, concentrated to dryness, co-evaporated with 50 mL of dichloromethane, concentrated to dryness, crystallized with ethyl acetate/petroleum ether, filtered, and dried to give a white solid (3.6 g, 84% yield). MS-ESI (m/z): [M+H]⁺ calcd for C₁₄H₁₉N₂O₅, 295.12; found, 295.12.

### Example 64. Synthesis of tert-butyl L-alanyl-L-alaninate (61)

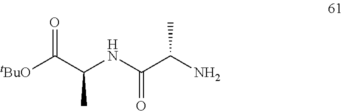

Compound 59 (10.0 g, 29.0 mmol) was dissolved in THF (80 mL), and 10% Pd/C (1.1 g) was added. The reaction flask was evacuated and back-filled with hydrogen for three times, and then stirred under a hydrogen balloon at r.t. for 4 h, and at 45-50° C. for 2 h, then filtered and concentrated to dryness. 2M HCl in ethyl acetate was added and the solution was evaporated to dryness, to give compound 61 as a white solid (5.8 g, 80% yield). MS-ESI (m/z): [M+H]⁺ calcd for C₁₀H₂₁N₂O₃, 217.15; found, 217.15.

### Example 65. Synthesis of tert-butyl ((benzyloxy)carbonyl)-L-alanyl-L-alanyl-L-alanyl-L-alaninate (62)

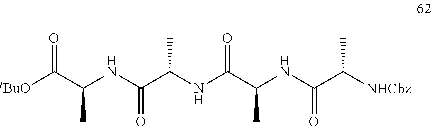 [SMILES: C[C@H](NC(=O)OCc1ccccc1)C(=O)N[C@@H](C)C(=O)N[C@@H](C)C(=O)N[C@@H](C)C(=O)OC(C)(C)C]

Compound 60 (3.0 g, 10.2 mmol) and compound 61 (3.0 g, 13.8 mmol) were dissolved in dichloromethane (50 mL), to which EDC (3.91 g, 20.4 mmol) and diisopropylethylamine (2.67 g, 20.4 mmol) were added, the reaction was stirred at 0° C. for 1 h, filtered, and concentrated to give a gray solid (5.0 g, 100% yield). MS-ESI (m/z): [M+H]⁺ calcd for C₂₄H₃₇N₄O₇, 493.26; found, 493.26.

### Example 66. Synthesis of tert-butyl L-alanyl-L-alanyl-L-alanyl-L-alaninate (63)

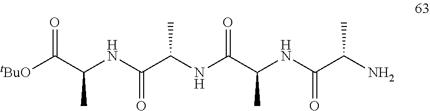 [SMILES: C[C@H](N)C(=O)N[C@@H](C)C(=O)N[C@@H](C)C(=O)N[C@@H](C)C(=O)OC(C)(C)C]

The crude compound 62 (5.0 g, 10.0 mmol) was dissolved in methanol (160 mL), and 10% Pd/C (1.1 g) was added. The reaction flask was evacuated and back-filled with hydrogen for three times, and then stirred under a hydrogen balloon at r.t. for 1 h, filtered, and concentrated to dryness to give an oil (3.0 g, 82% yield). MS-ESI (m/z): [M+H]⁺ calcd for C₁₆H₃₁N₄O₅, 359.22; found, 359.22.

### Example 67. Synthesis of tert-butyl ((S)-37-(((benzyloxy)carbonyl)amino)-31-oxo-2,5,8,11,14,17,20,23,26,29-decaoxa-32-azaoctatriacontan-38-oyl)-L-alanyl-L-alanyl-L-alanyl-L-alaninate (64)

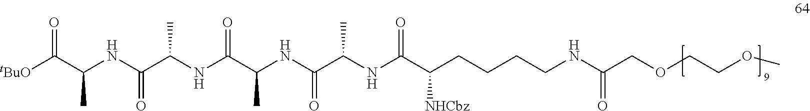

Compound 63 (1.8 g, 2.404 mmol) and compound 20 (1.0 g, 2.874 mmol) were dissolved in THF (30 mL), HATU (1.36 g, 3.592 mmol) and diisopropylethylamine (0.7 g, 4.789 mmol) were added. The reaction was stirred at r.t. for 1 h, concentrated, diluted with 20 mL of water and 25 mL of dichloromethane, the separated organic phase was washed with 5% Na₂CO₃, 1M HCl, dried over anhydrous sodium sulfate, concentrated to dryness and purified by preparative HPLC to give a colorless liquid (1.5 g, 57% yield). MS-ESI (m/z): [M+H]⁺ calcd for C₅₁H₈₉N₆O₁₉, 1089.61; found, 1089.61.

### Example 68. Synthesis of tert-butyl ((S)-37-amino-31-oxo-2,5,8,11,14,17,20,23,26,29-decaoxa-32-azaoctatriacontan-38-oyl)-L-alanyl-L-alanyl-L-alanyl-L-alaninate (65)

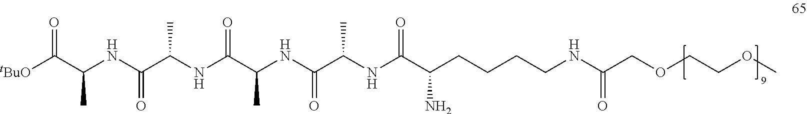

Compound 64 (1.0 g, 0.918 mmol) was dissolved in methanol (100 mL), 10% Pd/C (0.23 g) was added and the reaction flask was evacuated and back-filled with hydrogen for three times, and then stirred under a hydrogen balloon at r.t. for 2 h, filtered, and concentrated to dryness to give an oil 10 (0.9 g, 100% yield). MS-ESI (m/z): [M+H]⁺ calcd for C₄₃H₈₃N₆O₁₇, 955.57; found, 955.57.

### Example 69. Synthesis of di-tert-butyl (2S,5S,8S,11S,14S,22S,23S,31S,34S,37S,40S,43S)-22,23-bis(2,5-dioxo-2,5-dihydro-1H-pyrrol-1-yl)-2,5,8,11,34,37,40,43-octamethyl-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioate (66)

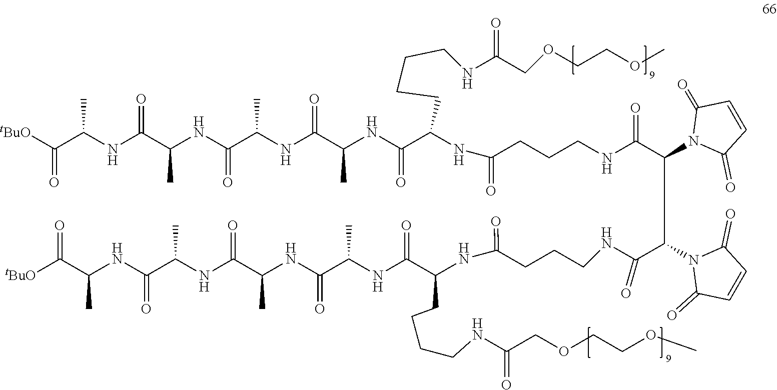

Compound 19 (204.9 mg, 0.428 mmol) and compound 65 (880 mg, 0.921 mmol) were dissolved in THF (10 mL) and DMF (10 mL), HATU (485 mg, 1.27 mmol) and diisopropylethylamine (216.5 mg, 1.27 mmol) were added, the reaction was stirred at r.t. for about 30 min, concentrated, diluted with dichloromethane (40 mL) and washed with 30 mL of brine. The aqueous phase was extracted twice with 100 mL of dichloromethane, the organic phases were combined, dried over anhydrous sodium sulfate, filtered, concentrated to dryness, to give a colorless oil. (985 mg, 100% yield). MS-ESI (m/z): [M+H]⁺ calcd for C₁₀₆H₁₈₃N₁₆O₄₂, 2352.26; found, 2352.26.

### Example 70. Synthesis of (2S,5S,8S,11S,14S,22S,23S,31S,34S,37S,40S,43S)-22,23-bis(2,5-dioxo-2,5-dihydro-1H-pyrrol-1-yl)-2,5,8,11,34,37,40,43-octamethyl-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioic acid (67)

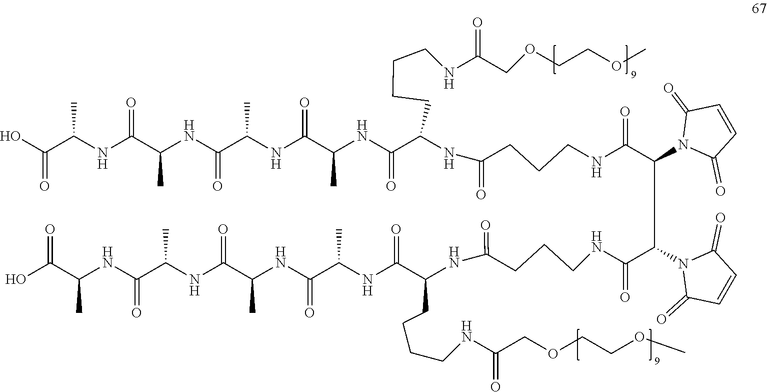

Compound 66 (985 mg, 0.419 mmol) was dissolved in dichloromethane (10 mL) and 20 mL of formic acid, reacted at 55-60° C. for 3 h, concentrated to dryness, and purified by preparative HPLC to give a colorless oil (0.6 g, 64% yield). MS-ESI (m/z): [M+H]⁺ calcd for C₉₈H₁₆₇N₁₆O₄₂, 2240.13; found, 2240.13.

### Example 71. Synthesis of (2S,3S)-2,3-bis(2,5-dioxo-2,5-dihydro-1H-pyrrol-1-yl)-N1,N4-bis((S)-37-(((S)-1-(((S)-1-(((S)-1-(((S)-1-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)amino)-1-oxopropan-2-yl)carbamoyl)-31,39-dioxo-2,5,8,11,14,17,20,23,26,29-decaoxa-32,38-diazadotetracontan-42-yl)succinamide (68a); (2S,3S)-2,3-bis(2,5-dioxo-2,5-dihydro-1H-pyrrol-1-yl)-N1,N4-bis((S)-37-(((4S,7S,10S,13S)-1-((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)-4,7,10-trimethyl-3,6,9,12-tetraoxo-2,5,8,11-tetraazatetradecan-13-yl)carbamoyl)-31,39-dioxo-2,5,8,11,14,17,20,23,26,29-decaoxa-32,38-diazadotetracontan-42-yl)succinamide (68b); and (2S,3S)-2,3-bis(2,5-dioxo-2,5-dihydro-1H-pyrrol-1-yl)-N1,N4-bis((S)-37-(((S)-1-(((S)-1-(((S)-1-(((S)-1-(4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperazin-1-yl)-1-oxopropan-2-yl)amino)-1-oxopropan-2-yl)amino)-1-oxopropan-2-yl)amino)-1-oxopropan-2-yl)carbamoyl)-31,39-dioxo-2,5,8,11,14,17,20,23,26,29-decaoxa-32,38-diazadotetracontan-42-yl)succinamide (68c)

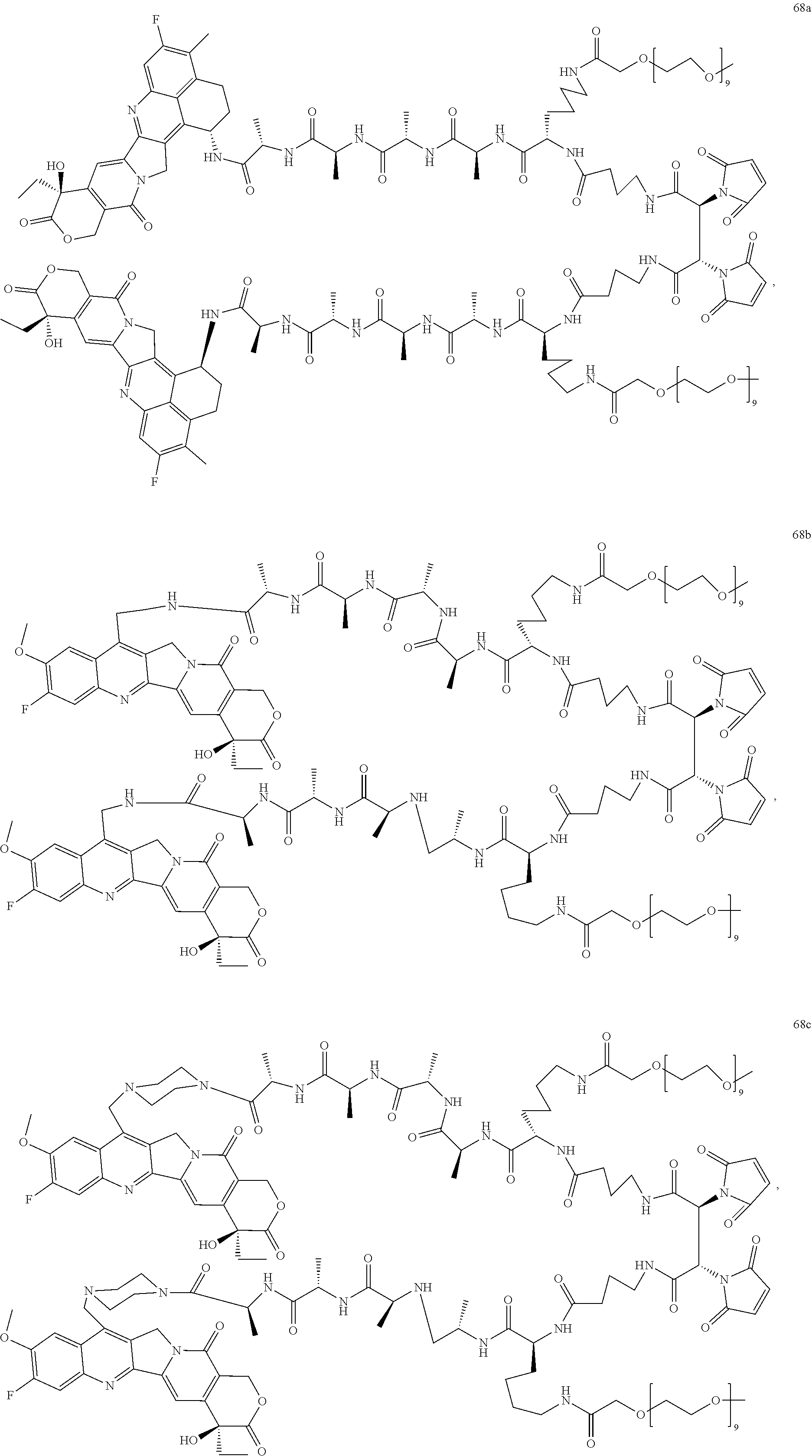

To a solution of compound 67 (202.2 mg, 0.090 mmol) HATU (110.3 mg, 0.290 mmol) and diisopropylethylamine (46.5 mg, 0.360 mmol) in DMF (10 mL) were added respectively exatecan mesylate (98.5 mg, 0.185 mmol), (S)-11-(aminomethyl)-4-ethyl-8-fluoro-4-hydroxy-9-methoxy-1H-pyrano[3′,4′:6,7]indolizino[1,2-b]quinoline-3,14(4H,12H)-dione, HCl salt (86.2 mg, 0.187 mmol) or(S)-4-ethyl-8-fluoro-4-hydroxy-9-methoxy-11-(piperazin-1-ylmethyl)-1H-pyrano[3′,4′:6,7]indolizino[1,2-b]quinoline-3,14(4H,12H)-dione, HCl salt (98.8 mg, 0.186 mmol). The reactions were stirred at r.t. for about 3 h, then concentrated, and purified by preparative HPLC, and lyophilized to give respectively compound 68a as a light yellow solid (189.1 mg, 67% yield). MS-ESI (m/z): [M+H]⁺ calcd for C₁₄₆H₂₀₇F₂N₂₂O₄₈, 3074.73; found, 3074.73; 68b as a light yellow solid (192.2 mg, 70% yield). MS-ESI (m/z): [M+H]⁺ calcd for C₁₄₂H₂₀₃F₂N₂₂O₅₀, 3054.40; found, 3054.90; or 68c as a light yellow solid (205.8 mg, 71% yield). MS-ESI (m/z): [M+H]⁺ calcd for C₁₅₀H₂₁₇F₂N₂₄O₅₀, 3192.51; found, 3192.95.

### Example 72. Synthesis of di-tert-butyl 5,5′-((((10S,18S,19S,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3,8,11,16,21,26,29,34-octaazahexatriacontanedioyl)bis(azanediyl))bis(4-hydroxy-3,1-phenylene))(4R,4′R)-bis(4-((tert-butoxycarbonyl)amino)pentanoate) (70)

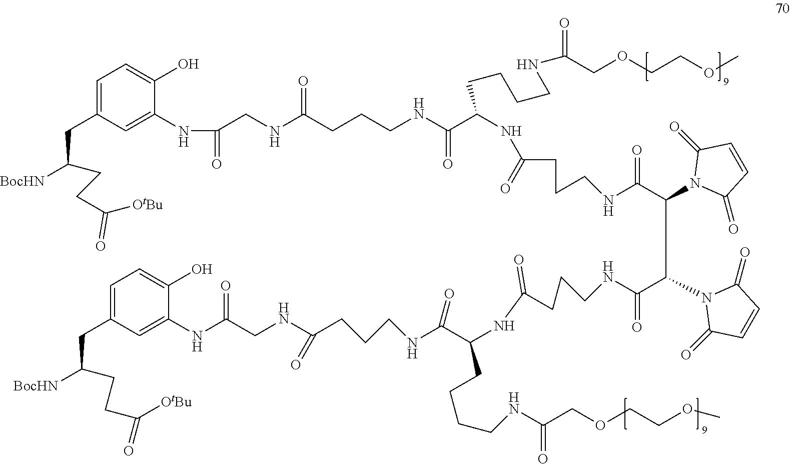

Compound 35 (720 mg, 0.368 mmol) and tert-butyl (R)-5-(3-amino-4-hydroxyphenyl)-4-((tert-butoxycarbonyl)amino)pentanoate (69, 350 mg, 0.920 mmol) were dissolved in dichloromethane (20 mL), to which EDCI (211 mg, 1.10 mmol) was added, and the reaction was stirred for 0.5 hours, and then concentrated to dryness. The crude product was purified by preparative HPLC (57% MeCN in H2O) to give compound 70 as an oil (200 mg, 0.075 mmol, 20.27%). MS-ESI (m/z): [M+2H]²⁺ calcd for C₁₂₆H₂₀₆N₁₆O₄₆, 1342.05; found, 1341.91.

### Example 73. Synthesis of (4R,4′R)-5,5′-((((10S,18S,19S,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3, 8,11,16,21,26,29,34-octaazahexatriacontanedioyl)bis(azanediyl))bis(4-hydroxy-3,1-phenylene))bis(4-aminopentanoic acid) (71)

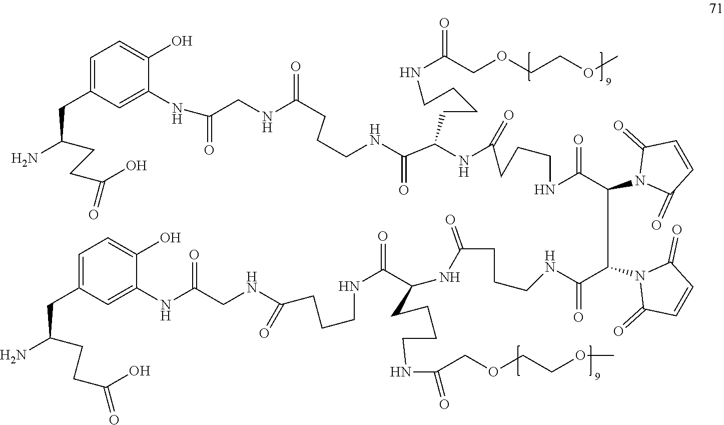

Compound 70 (200 mg, 0.075 mmol) was dissolved in dichloromethane (4 mL), trifluoroacetic acid (1 mL) was added, and the reaction was stirred overnight, and then concentrated, co-evaporated with dichloromethane twice, dried on an oil pump to give compound 71 as an oil (176.69 mg, 0.075 mmol, 100.00%) MS-ESI (m/z): [M+2H]²⁺ calcd for C₁₀₈H₁₇₄N₁₆O₄₂, 1184.60; found, 1185.17.

### Example 74. Synthesis of (4R,4′R)-5,5′-((((10S,18S,19S,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3, 8,11,16,21,26,29,34-octaazahexatriacontanedioyl)bis(azanediyl))bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)pentanoic acid) (72a); and (R,R,S,S,S,4R,4′R)-5,5′-((((105,185,195,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3,8,11,16,21,26,29,34-octaazahexatriacontane-1,36-dioyl)bis(azanediyl))bis(4-hydroxy-3,1-phenylene))bis(4-(2-((3S,6S,9R,11R)-6-((S)-sec-butyl)-3,9-diisopropyl-2,8-dimethyl-4,7,13-trioxo-12-oxa-2,5,8-triazatetradecan-11-yl)thiazole-4-carboxamido)pentanoic acid) (72b)

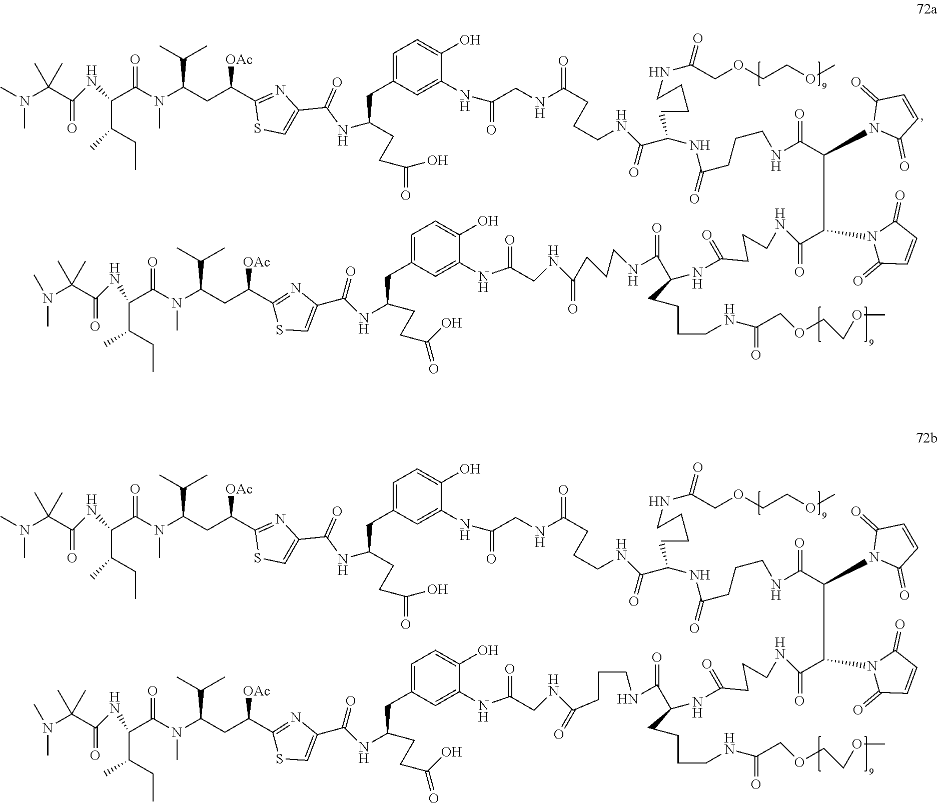

Compound 71 (88 mg, 0.037 mmol) in DMF (1 mL) and diisopropylethylamine (0.025 mL, 0.149 mmol) were added perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (Tub-1, 64 mg, 0.093 mmol) or perfluorophenyl 2-((3S,6S,9R,11R)-6-((S)-sec-butyl)-3,9-diisopropyl-2,8-dimethyl-4,7,13-trioxo-12-oxa-2,5,8-triazatetradecan-11-yl)thiazole-4-carboxylate (Tub-3, 67 mg, 0.095 mmol) respectively. The reactions were stirred for 4 hours and purified by preparative HPLC to give compound 72a as a solid (72 mg, 0.021 mmol, 57%). MS-ESI (m/z): [M+3H]³⁺ calcd for C₁₅₈H₂₅₄N₂₄O₅₂S₂, 1128.91; found, 1129.72, or compound 72b as a solid (69 mg, 0.020 mmol, 54%). MS-ESI (m/z): [M+3H]³⁺ calcd for C₁₆₀H₂₆₁N₂₄O₅₂S₂, 1138.26; found, 1139.15.

### Example 75. Synthesis of (4R,4′R)-5,5′-((((10S,18S,19S,27S)-18,19-bis(2,5-dioxo-2,5-dihydro-1H-pyrrol-1-yl)-4,9,12,17,20,25,28,33-octaoxo-10,27-bis(31-oxo-2,5,8,11,14,17,20,23,26,29-decaoxa-32-azahexatriacontan-36-yl)-3,8,11,16,21,26,29,34-octaazahexatriacontanedioyl)bis(azanediyl))bis(4-hydroxy-3,1-phenylene))bis(4-(2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxamido)pentanoic acid) (73)

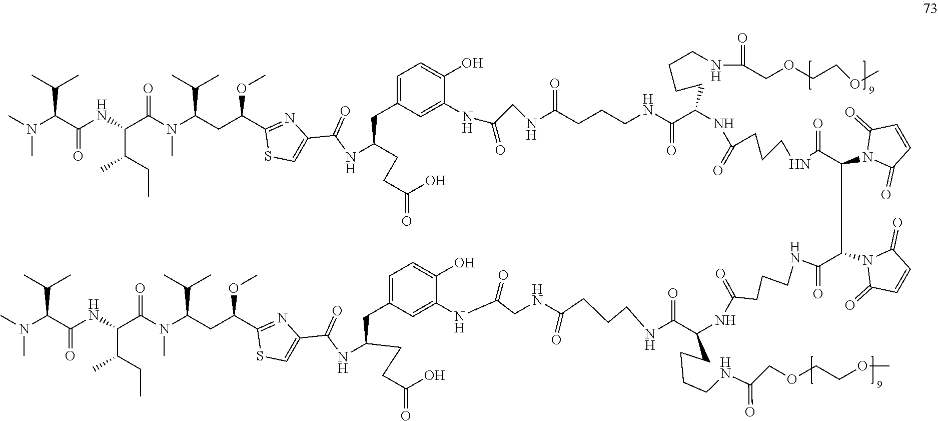

Compound 71 (83.75 mg, 0.035 mmol) and perfluorophenyl 2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxylate (Tub-2, 60 mg, 0.088 mmol) were dissolved in DMF (1.5 mL) and diisopropylethylamine (0.023 mL, 0.141 mmol) was added. The reaction was stirred for 2 hours and purified by preparative HPLC to give a solid compound 73 (54 mg, 0.016 mmol, 45%). MS-ESI (m/z): [M+3H]³⁺ calcd for C₁₅₈H₂₅₈N₂₄O₅₀S₂, 1120.68; found, 1120.48.

### Example 76. Synthesis of tert-butyl ((S)-37-(((benzyloxy)carbonyl)amino)-31-oxo-2,5,8,11,14,17,20,23,26,29-decaoxa-32-azaoctatriacontan-38-oyl)-L-valyl-L-alaninate (74)

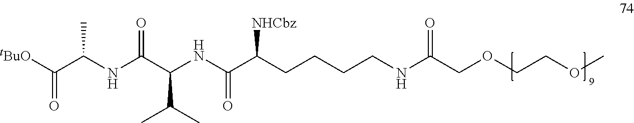

Compound 20 (8.0 g, 10.7 mmol) and H-Val-Ala-O^{t}Bu (2.7 g, 10.7 mmol) were dissolved in dichloromethane (150 mL), HATU (5.4 g, 13.9 mmol) and diisopropylethylamine (2.8 g, 21.4 mmol) were added and stirred at r.t. for 3 hours. The reaction solution was washed with brine, dried over anhydrous sodium sulfate, filtered and concentrated, and the residue was purified by silica gel column (dichloromethane:methanol=20:1) to give 9.4 g of the title compound with a yield of 90%. MS m/z: 976.1 ([M+H]⁺).

### Example 77. Synthesis of ((S)-37-(((benzyloxy)carbonyl)amino)-31-oxo-2,5,8,11,14,17,20,23,26,29-decaoxa-32-azaoctatriacontan-38-oyl)-L-valyl-L-alanine (75)

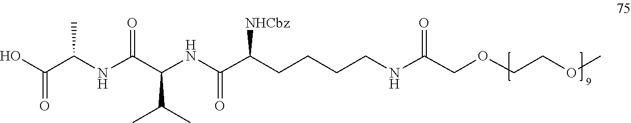

Compound 74 (9.4 g, 9.6 mmol) was dissolved in dichloromethane (50 mL), and treated with trifluoroacetic acid (50 mL) at r.t. for 1 hour. The reaction solution was concentrated to remove most of the trifluoroacetic acid, and diluted with 200 mL of dichloromethane, the solution was washed with brine, dried over anhydrous sodium sulfate, filtered and concentrated to give 8.1 g of the title compound with a yield of 82%. MS m/z: 919.9 ([M+H]⁺).

### Example 78. Synthesis of 2,5-dioxopyrrolidin-1-yl ((S)-37-(((benzyloxy)carbonyl)amino)-31-oxo-2,5,8,11,14,17,20,23,26,29-decaoxa-32-azaoctatriacontan-38-oyl)-L-valyl-L-alaninate (76)

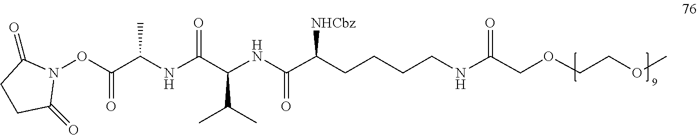

Compound 75 (8.1 g, 8.8 mmol) was dissolved in dichloromethane (120 mL), EDCI (6.1 g, 32 mmol) and NHS (2.5 g, 21.3 mmol) were added at 0° C., and the reaction was stirred at 0° for 1 hour, and then washed with brine, dried over anhydrous sodium sulfate, filtered and concentrated to give 8.1 g of the title compound (91% yield). MS m/z: 1017.1 ([M+H]⁺).

### Example 79. Synthesis of (2S,4R)-5-(3-((37S,40S,43S)-37-(((benzyloxy)carbonyl)amino)-40-isopropyl-43-methyl-31,38,41-trioxo-2,5,8,11,14,17,20,23,26,29-decaoxa-32,39,42-triazatetratetracontan-44-amido)-4-hydroxyphenyl)-4-((tert-butoxycarbonyl)amino)-2-methylpentanoic acid (78)

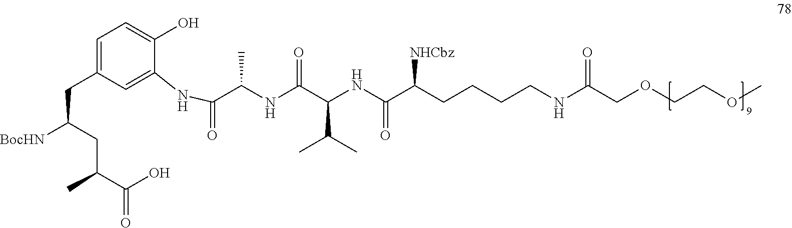

Compound 76 (8.1 g, 7.9 mmol) and (2S,4R)-5-(3-amino-4-hydroxyphenyl)-4-((tert-butoxycarbonyl)amino)-2-methylpentanoic acid (77, 2.7 g. 7.9 mmol) were dissolved in THF (150 mL), heated to 50° C. and stirred overnight. The reaction was concentrated and the residue was purified by silica gel column (dichloromethane:methanol=20:1) to give 8.0 g of the title compound (82% yield). MS m/z: 1240.1 ([M+H]⁺).

### Example 80. Synthesis of (2S,4R)-5-(3-((37S,40S,43S)-37-amino-40-isopropyl-43-methyl-31,38,41-trioxo-2,5,8,11,14,17,20,23,26,29-decaoxa-32,39,42-triazatetratetracontan-44-amido)-4-hydroxyphenyl)-4-((tert-butoxycarbonyl)amino)-2-methylpentanoic acid (79)

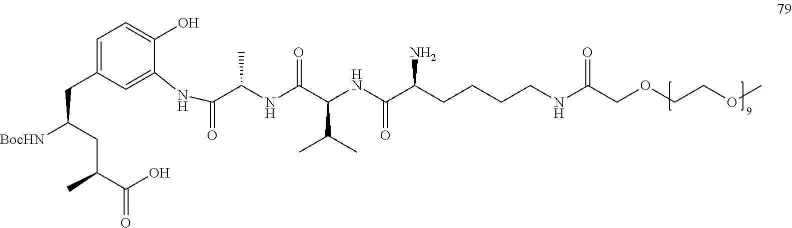

Compound 78 (8.0 g, 6.4 mmol) was dissolved in isopropanol (100 mL), palladium on carbon (10 wt %, 2 g) was added, and the reaction flask was evacuated and back-filled with hydrogen for three times, heated at 50° C. for 4 hours. The reaction mixture was filtered, and the filtrate was concentrated to give 6.8 g of the title compound (96% yield). MS m/z: 1106.1 ([M+H]⁺).

### Example 81. Synthesis of bis(2,5-dioxopyrrolidin-1-yl) 4,4′-(((2S,3S)-2,3-bis(2,5-dioxo-2,5-dihydro-1H-pyrrol-1-yl)succinyl)bis(azanediyl))dibutyrate (80)

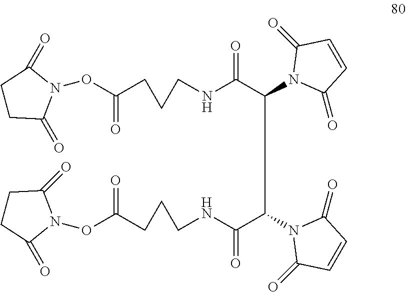 [SMILES: O=C(CCCNC(=O)[C@H]([C@@H](C(=O)NCCCC(=O)ON1C(=O)CCC1=O)N1C(=O)C=CC1=O)N1C(=O)C=CC1=O)ON1C(=O)CCC1=O]

Compound 19 (2.40 g, 5.0 mmol) was dissolved in DMF (100 mL), EDCI (2.86 g, 15 mmol) and NHS (1.41 g, 12 mmol) were added at 0° C., and the reaction was stirred at 0° for 1 hour, and then concentrated. The residue was diluted with dichloromethane, washed with brine, dried over anhydrous sodium sulfate, filtered and concentrated to give 3.19 g of the title compound (95% yield).

### Example 82. Synthesis of (2S,2'S,4R,4′R)-5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis(azanediyl)) bis(4-hydroxy-3,1-phenylene))bis(4-((tert-butoxycarbonyl)amino)-2-methylpentanoic acid) (81)

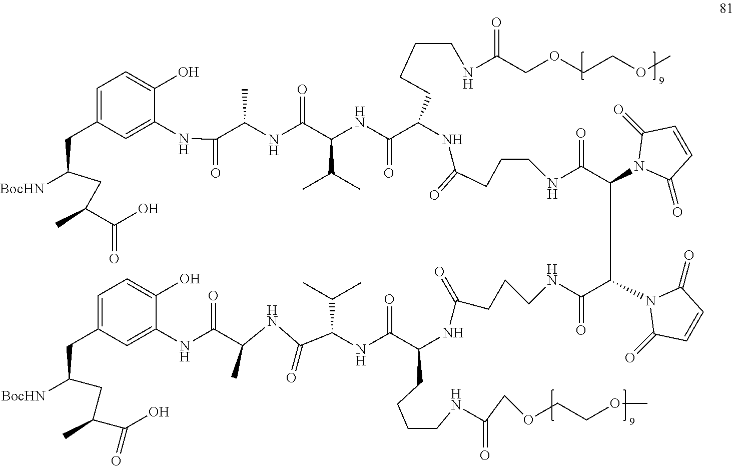

Compound 80 (400 mg, 0.6 mmol) and compound 79 (1.3 g, 1.2 mmol) were dissolved in THF (15 mL) and heated to 50° C., stirred overnight. The reaction solution was concentrated and the residue was purified by preparative HPLC, and 670 mg of the title compound was obtained after lyophilization (42% yield).

MS: m/z=1328.0 [½M+H⁺]

### Example 83. Synthesis of (2S,2'S,4R,4′R)-5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis(azanediyl))bis(4-hydroxy-3,1-phenylene))bis(4-amino-2-methylpentanoic acid) (82)

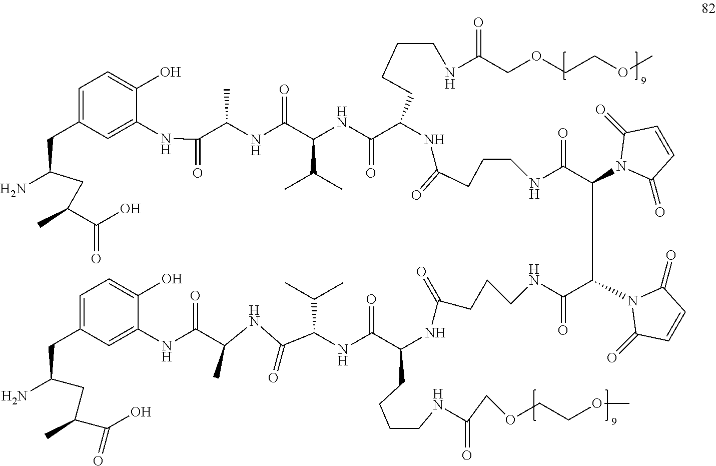

Compound 81 (670 mg, 0.25 mmol) was dissolved in dichloromethane (8 mL), and stirred with trifluoroacetic acid (4 mL) at r.t. for 1 hour. The reaction was concentrated, the residue was purified by preparative HPLC, and 500 mg of the title compound was obtained after lyophilization (80% yield). MS m/z: 1227.1 ([M+2H]²⁺).

### Example 84. Synthesis of (25,2'S,4R,4′R)-5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis(azanediyl)) bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid) (83)

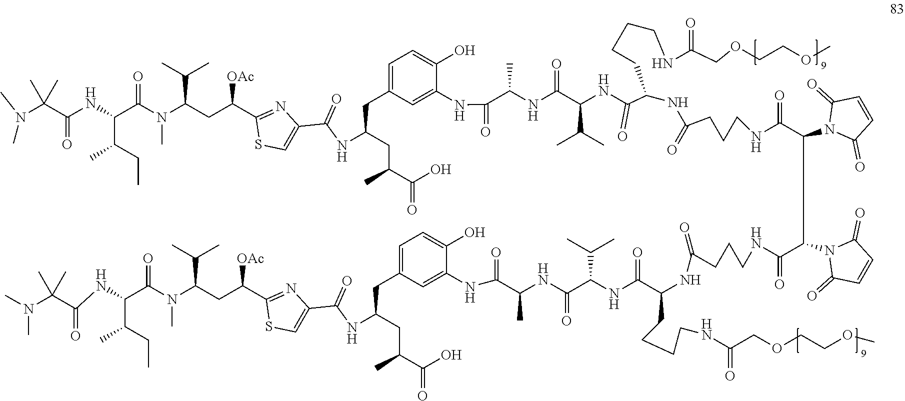

To a solution of compound 82 (200 mg, 0.08 mmol) and Tub-1 (120 mg, 0.16 mmol) in DMF (2 mL), diisopropylethylamine (30 mg, 0.24 mmol) was added, and the reaction was stirred at r.t. for 1 hour. The reaction solution was then purified by preparative HPLC, and 50 mg of product was obtained after lyophilization (18% yield). MS m/z: 1736.1 ([M+2H]²⁺).

### Example 85. Synthesis of tert-butyl (R)-5-(3-(2-(((benzyloxy)carbonyl)amino)acetamido)-4-hydroxyphenyl)-4-((tert-butoxycarbonyl)amino)pentanoate (84)

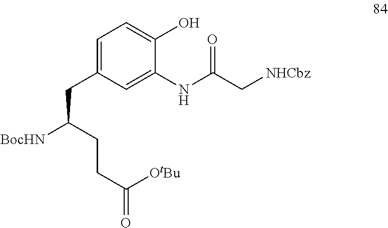 [SMILES: CC(C)(C)OC(=O)CC[C@H](Cc1ccc(O)c(NC(=O)CNC(=O)OCc2ccccc2)c1)NC(=O)OC(C)(C)C]

Cbz-Gly-OH (2.09 g, 10.0 mmol) was dissolved in dichloromethane (150 mL), compound 69 (4.19 g, 11.0 mmol) and EDCI (3.83 g, 20.0 mmol) were added. The reaction mixture was stirred at r.t. for 3 hours, washed with brine, dried over anhydrous sodium sulfate, filtered and concentrated, the crude product was purified by silica gel column (20%-50% EA/PE) to give compound 84 (4.88 g, 85% yield).

### Example 86. Synthesis of tert-butyl (R)-5-(3-(2-aminoacetamido)-4-hydroxyphenyl)-4-((tert-butoxycarbonyl)amino)pentanoate (85)

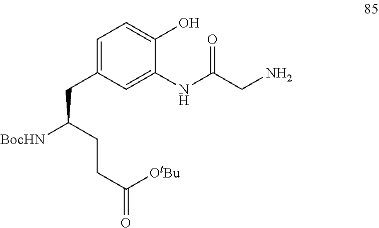 [SMILES: CC(C)(C)OC(=O)CC[C@H](Cc1ccc(O)c(NC(=O)CN)c1)NC(=O)OC(C)(C)C]

Compound 84 (1.44 g, 2.52 mmol) was dissolved in methanol (40 mL) and Pd/C (10% wet, 0.3 g) was added. The reaction flask was evacuated and back-filled with hydrogen for three times, and then stirred under a hydrogen balloon for 6 hours, filtered, concentrated, and co-evaporated with 50 mL of dichloromethane, dried on an oil pump to give compound 85 (0.9 g, 82% yield).

### Example 87. Synthesis of di-tert-butyl 5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)bis(azanediyl))bis(4-hydroxy-3,1-phenylene))(4R,4′R)-bis(4-((tert-butoxycarbonyl)amino)pentanoate) (86)

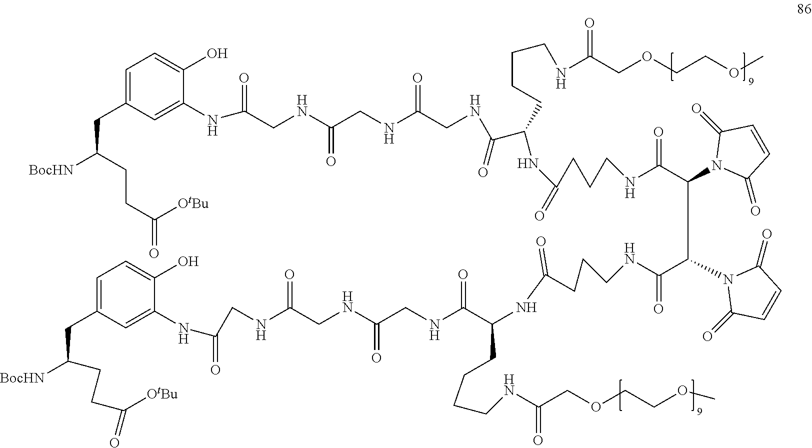

In a 100 mL single-necked reaction flask, compound 29 (840 mg, 0.44 mmol) was dissolved in dichloromethane (50 mL) and the mixture was magnetically stirred at r.t., then compound 85 (391 mg, 0.89 mmol) and EDC (362 mg, 1.89 mmol) were added, and the reaction was carried out at r.t. for 1 hour, then washed with brine, dried over anhydrous sodium sulfate, filtered and concentrated. The crude product was purified by preparative HPLC (acetonitrile/water) to give compound 86 (244 mg, 20% yield). MS-ESI (m/z): [M+H]⁺ calcd for C₁₂₆H₂₀₄N₁₈O₄₈, 1370.55; found, 1370.56.

### Example 88. Synthesis of (4R,4′R)-5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)bis(azanediyl))bis(4-hydroxy-3,1-phenylene))bis(4-aminopentanoic acid) (87)

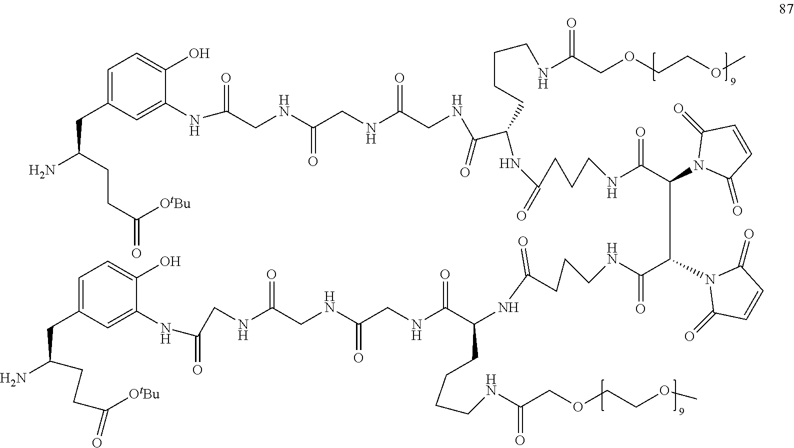

In a 100 mL one-neck reaction flask, compound 86 (244 mg, 0.089 mmol), dichloromethane (8 mL) and trifluoroacetic acid (8 mL) were stirred at r.t. for 1 hour. The reaction mixture was concentrated under reduced pressure, and placed on an oil pump, to give compound 87, which was used without further purification, assuming 100% yield. MS-ESI (m/z): [M+H]⁺ calcd for C₁₀₈H₁₇₂N₁₈O₄₄, 1214.33; found, 1214.02.

### Example 89. Synthesis of (4R,4′R)-5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)bis(azanediyl))bis(4-hydroxy-3,1-phenylene))bis(4-(2-((6S,9R,11R)-6-((S)-sec-butyl)-9-isopropyl-2,3,3, 8-tetramethyl-4,7,13-trioxo-12-oxa-2,5,8-triazatetradecan-11-yl)thiazole-4-carboxamido)pentanoic acid) (88)

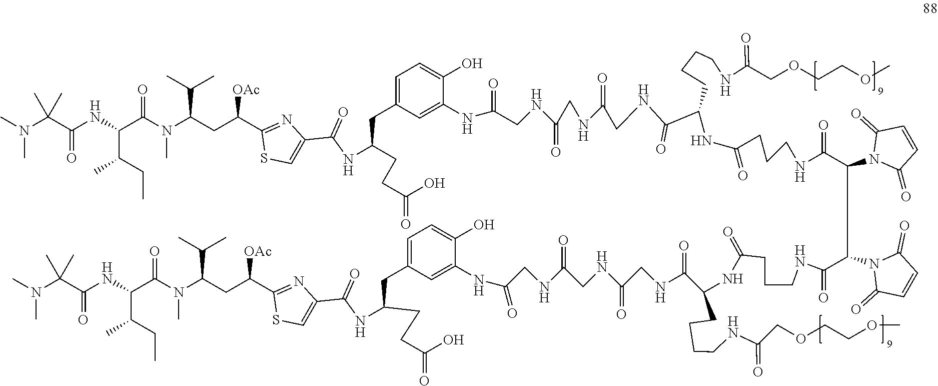

In a 100 mL single-necked reaction flask, to a solution of compound 87 (the crude product from previous step, 0.089 mmol) and Tub-1 (128 mg, 0.185 mmol) in DMF (10 mL), was added dropwise diisopropylethylamine (128 mg, 0.990 mmol). After stirring at r.t. for 5 hours, the reaction was concentrated under reduced pressure. The residue was diluted with 10 mL of dichloromethane, 0.2 mL of formic acid was added dropwise, and the mixture was concentrated, then purified by preparative HPLC (acetonitrile/water), to give compound 88 (40 mg, 13% yield). MS-ESI (m/z): [M+2H]²⁺ calcd for C₁₅₈H₂₅₂N₂₆O₅₄S₂, 1723.00; found, 1722.84.

### Example 90. Synthesis of di-tert-butyl 5,5′-((((14S,22S,23S,31S)-22,23-bis(2,5-dioxo-2,5-dihydro-1H-pyrrol-1-yl)-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioyl)bis(azanediyl))bis(4-hydroxy-3,1-phenylene))(4R,4′R)-bis(4-((tert-butoxycarbonyl)amino)pentanoate) (89)

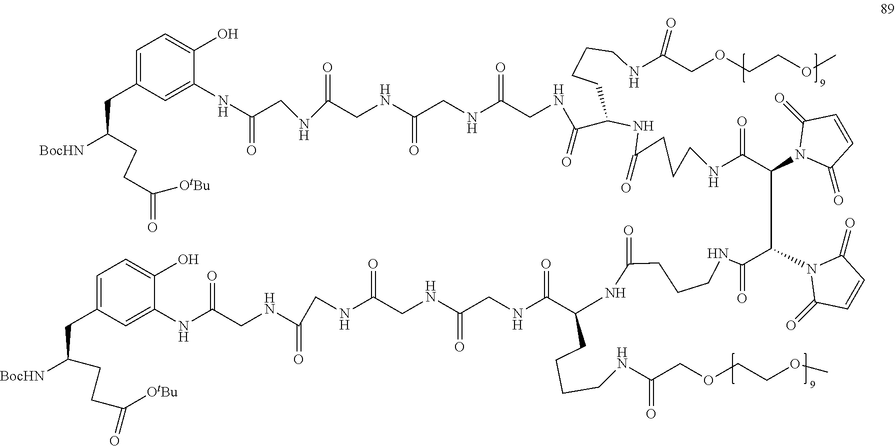

To a solution of compound 43 (350 mg, 0.17 mmol) and compound 85 (170 mg, 0.38 mmol) in dichloromethane (5 mL), EDCI (100 mg, 0.52 mmol) was added, and the reaction was stirred at r.t. for 2 hours. LCMS as indicated completion of the reaction. And the reaction solution was washed with brine, dried over anhydrous sodium sulfate, filtered and concentrated. The residue was purified by preparative HPLC (acetonitrile/water containing 0.1% HCOOH) to give 170 mg of the title compound (34% yield).

### Example 91. Synthesis of (4R,4′R)-5,5′-((((14S,22S,23S,31S)-22,23-bis(2,5-dioxo-2,5-dihydro-1H-pyrrol-1-yl)-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioyl)bis(azanediyl))bis(4-hydroxy-3,1-phenylene))bis(4-aminopentanoic acid) (90)

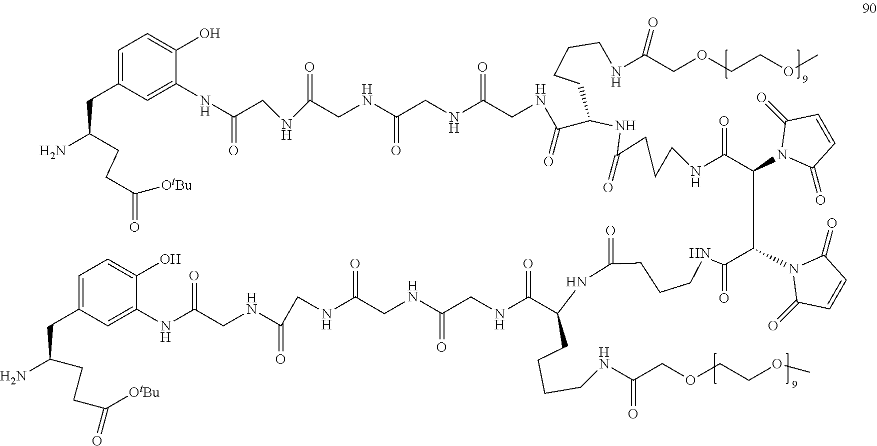

Compound 89 (170 mg, 0.06 mmol) was dissolved in dichloromethane (4 mL) and reacted with trifluoroacetic acid (2 mL) at r.t. for 2 hours. The reaction mixture was concentrated and purified by preparative HPLC (acetonitrile/water containing 0.1% HCOOH) to give 140 mg of the title compound (93% yield). MS m/z: 1271.0 ([M+2H]²⁺).

### Example 92 Synthesis of (4R,4′R)-5,5′-((((14S,22S,23S,31S)-22,23-bis(2,5-dioxo-2,5-dihydro-1H-pyrrol-1-yl)-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioyl)bis(azanediyl))bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)pentanoic acid) (91)

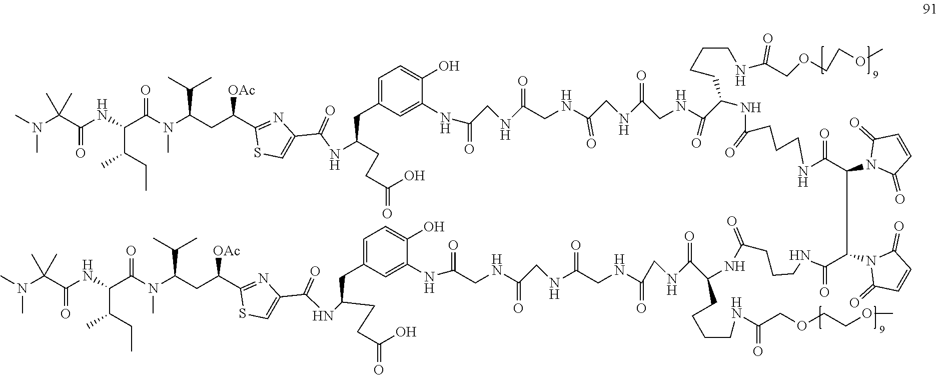

Compound 90 (140 mg, 0.05 mmol) and Tub-1 (95 mg, 0.14 mmol) were dissolved in dichloromethane (5 mL), and DIEA (20 mg) was then added to the solution and stirred at r.t. for 1 hour. LCMS as indicated completion of the reaction. And the reaction solution was concentrated and purified by preparative HPLC (acetonitrile/water containing 0.100 HCOOH) to give 150 mg of the title compound (75% yield). MS m/z: 1780.0 ([M+2H]²⁺).

### Example 93. Synthesis of di-tert-butyl (13S,21S,22S,30S)-21,22-bis(2,5-dioxo-2,5-dihydro-1H-pyrrol-1-yl)-4,7,12,15,20,23,28,31,36,39-decaoxo-13,30-bis(31-oxo-2,5,8,11,14,17,20,23,26,29-decaoxa-32-azahexatriacontan-36-yl)-3,6,11,14,19,24,29,32,37,40-decaazadotetracontanedioate (92)

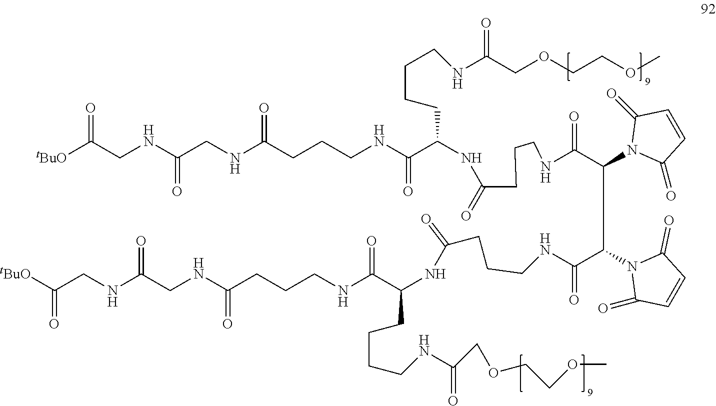

To a solution of compound 35 (870 mg, 0.445 mmol) in dichloromethane (30 mL), pentafluorophenol (245 mg, 1.334 mmol) and DIC (224 mg, 1.779 mmol) were added. After stirring for 1 hour, H-Gly-OtBu·HCl (164 mg, 0.978 mmol) and diisopropylethylamine (0.294 mL, 1.779 mmol) were added. The reaction was stirred for 25 minutes, and then washed with 20 mL of brine, dried over anhydrous sodium sulfate and concentrated to give compound 92 as an oil (970 mg, 0.444 mmol, 99%). MS-ESI (m/z): [M+H]⁺ calcd for C₉₈H₁₆₈N₁₄O₄₀, 2183.48; found, 2185.45.

### Example 94. Synthesis of (13S,21S,22S,30S)-21,22-bis(2,5-dioxo-2,5-dihydro-1H-pyrrol-1-yl)-4,7,12,15,20,23,28,31,36,39-decaoxo-13,30-bis(31-oxo-2,5,8,11,14,17,20,23,26,29-decaoxa-32-azahexatriacontan-36-yl)-3,6,11,14,19,24,29,32,37,40-decaazadotetracontanedioic acid (93)

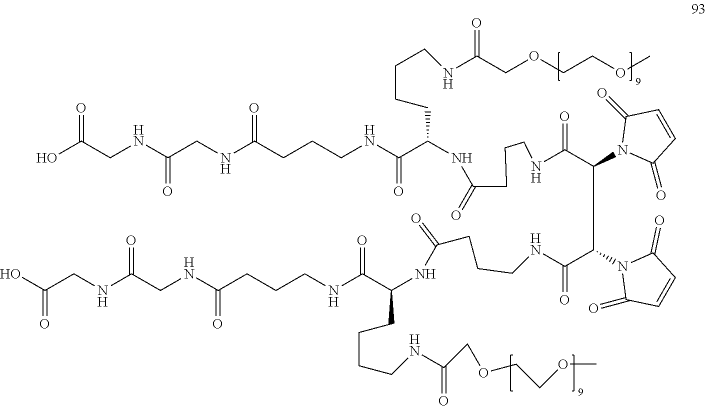

Compound 92 (0.97 g, 0.444 mmol) was dissolved in dichloromethane (10 mL) and treated with trifluoroacetic acid (5 mL). The reaction solution was stirred overnight, concentrated and purified by preparative HPLC to give an oil (636 mg, 0.307 mmol, 69%). MS-ESI (m/z): [M+H]⁺ calcd for C₉₀H₁₅₂N₁₄O₄₀, 2071.26; found, 2071.72.

### Example 95. Synthesis of di-tert-butyl 5,5′-((((16S,24S,25S,33S)-24,25-bis(2,5-dioxo-2,5-dihydro-1H-pyrrol-1-yl)-4,7,10,15,18,23,26,31,34,39,42,45-dodecaoxo-16,33-bis(31-oxo-2,5,8,11,14,17,20,23,26,29-decaoxa-32-azahexatriacontan-36-yl)-3,6,9,14,17,22,27,32,35,40,43,46-dodecaazaoctatetracontanedioyl)bis(azanediyl))bis(4-hydroxy-3,1-phenylene))(4R,4′R)-bis(4-((tert-butoxycarbonyl)amino)pentanoate) (94)

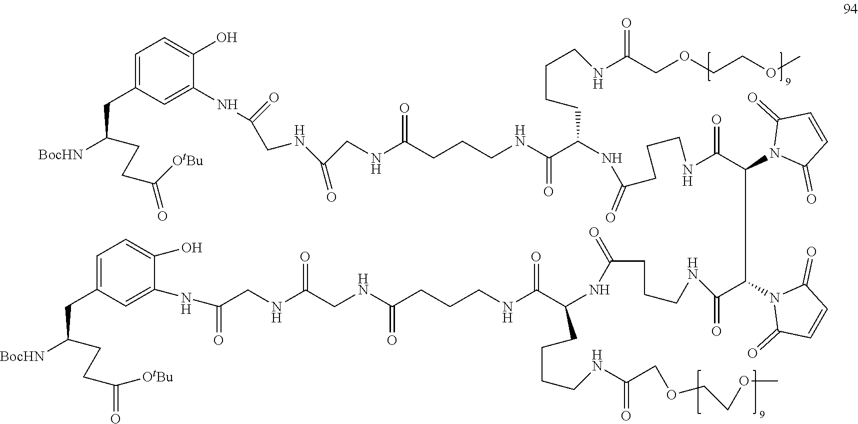

To a solution of compound 93 (636 mg, 0.307 mmol) in dichloromethane (20 mL), compound 85 (296 mg, 0.676 mmol) and EDCI (177 mg, 0.922 mmol) were added. After 1 hour, the reaction mixture was washed with 20 mL of brine, dried over anhydrous sodium sulfate and concentrated, purified by preparative HPLC (acetonitrile/water) to give compound 94 as an oil (263 mg, 0.090 mmol, 29%). MS-ESI (m/z): [M+H]⁺ calcd for C₁₃₄H₂₁₈N₂₀O₅₀, 1456.15; found, 1455.84.

### Example 96. Synthesis of (4R,4′R)-5,5′-((((16S,24S,25S,33S)-24,25-bis(2,5-dioxo-2,5-dihydro-1H-pyrrol-1-yl)-4,7,10,15,18,23,26,31,34,39,42,45-dodecaoxo-16,33-bis(31-oxo-2,5,8,11,14,17,20,23,26,29-decaoxa-32-azahexatriacontan-36-yl)-3,6,9,14,17,22,27,32,35,40,43,46-dodecaazaoctatetracontanedioyl)bis(azanediyl))bis(4-hydroxy-3,1-phenylene))bis(4-aminopentanoic acid) (95)

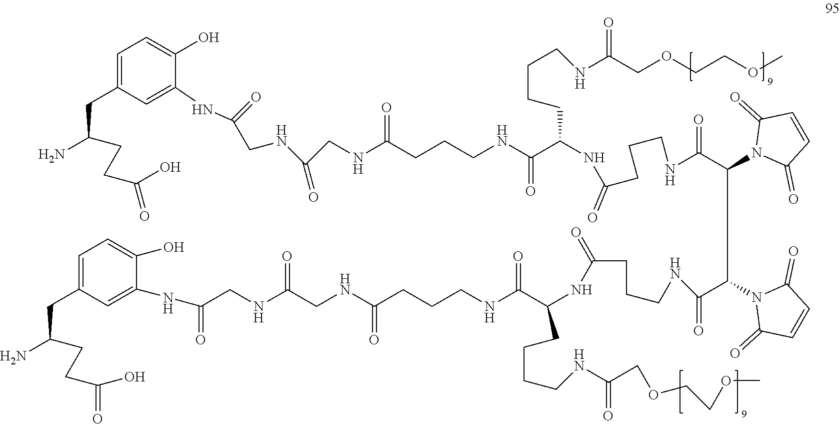

Compound 94 (263 mg, 0.090 mmol) was dissolved in dichloromethane (12 mL) and treated with trifluoroacetic acid (6 mL). The reaction was stirred for 5 hours, and concentrated to dryness. Compound 95 was obtained as an oil (234 mg, 0.090 mmol, 99%). MS-ESI (m/z): [M+H]⁺ calcd for C₁₁₆H₁₈₆N₂₀O₄₆, 1299.93; found, 1299.77.

### Example 97. Synthesis of (4R,4′R)-5,5′-((((16S,24S,25S,33S)-24,25-bis(2,5-dioxo-2,5-dihydro-1H-pyrrol-1-yl)-4,7,10,15,18,23,26,31,34,39,42,45-dodecaoxo-16,33-bis(31-oxo-2,5,8,11,14,17,20,23,26,29-decaoxa-32-azahexatriacontan-36-yl)-3,6,9,14,17,22,27,32,35,40,43,46-dodecaazaoctatetracontanedioyl)bis(azanediyl))bis(4-hydroxy-3,1-phenylene))bis(4-(2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxamido)pentanoic acid) (96)

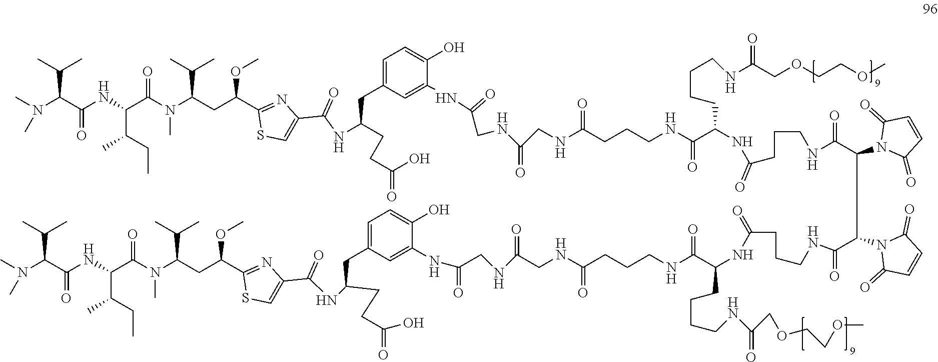

To a solution of 95 (234 mg, 0.090 mmol) and Tub-2 (152 mg, 0.225 mmol) in DMF (2 mL), diisopropylethylamine (0.060 mL, 0.360 mmol) was added, and the reaction was stirred for 5 hours, and neutralized with formic acid. The mixture was purified by preparative HPLC (acetonitrile/water) to give compound 96 as a solid (100 mg, 0.028 mmol, 31%). MS-ESI (m/z). [M+2H]²⁺ calcd for C₁₆₆H₂₇₀N₂₈O₅₄S₂, 1794.63; found, 1794.83.

### Example 98. Synthesis of di-tert-butyl (2S,13S,21S,22S,30S,41S)-21,22-bis(2,5-dioxo-2,5-dihydro-1H-pyrrol-1-yl)-2,41-dimethyl-4,7,12,15,20,23,28,31,36,39-decaoxo-13,30-bis(31-oxo-2,5,8,11,14,17,20,23,26,29-decaoxa-32-azahexatriacontan-36-yl)-3,6,11,14,19,24,29,32,37,40-decaazadotetracontanedioate (97)

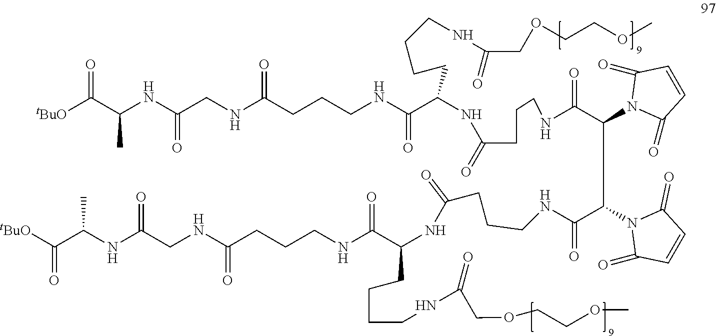

To a solution of compound 35 (827 mg, 0.423 mmol) in dichloromethane (30 mL), pentafluorophenol (233.46 mg, 1.268 mmol) and DIC (213.41 mg, 1.691 mmol) were added. After stirring for 1 hour, H-Ala-OtBu·HCl (169 mg, 0.930 mmol) and diisopropylethylamine (0.280 mL, 1.691 mmol) were added. The reaction was stirred for 1 hour and then washed with 20 mL of brine, dried over anhydrous sodium sulfate, filtered and concentrated. Compound 97 was obtained as an oil (934 mg, 0.423 mmol, 99.94%). MS-ESI (m/z): [M+H]⁺ calcd for C₁₀₀H₁₇₂N₁₄O₄₀, 2211.53; found, 2212.50.

### Example 99. Synthesis of (2S,13S,21S,22S,30S,41S)-21,22-bis(2,5-dioxo-2,5-dihydro-1H-pyrrol-1-yl)-2,41-dimethyl-4,7,12,15,20,23,28,31,36,39-decaoxo-13,30-bis(31-oxo-2,5,8,11,14,17,20,23,26,29-decaoxa-32-azahexatriacontan-36-yl)-3,6,11,14,19,24,29,32,37,40-decaazadotetracontanedioic acid (98)

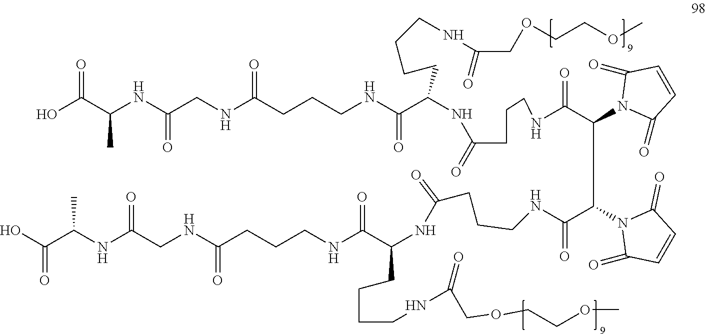

Compound 97 (0.93 g, 0.421 mmol) was dissolved in dichloromethane (10 mL) and treated with trifluoroacetic acid (5 mL) overnight. The reaction solution was concentrated and purified by preparative HPLC (acetonitrile/water) to give compound 98 as an oil (695 mg, 0.331 mmol, 79%). MS-ESI (m/z): [M+H]⁺ calcd for C₉₂H₁₅₆N₁₄O₄₀, 2099.32; found, 2100.87.

### Example 100. Synthesis of di-tert-butyl 5,5′-((((2S,5S,16S,24S,25S,33S,44S,47S)-24,25-bis(2,5-dioxo-2,5-dihydro-1H-pyrrol-1-yl)-2,5,44,47-tetramethyl-4,7,10,15,18,23,26,31,34,39,42,45-dodecaoxo-16,33-bis(31-oxo-2,5,8,11,14,17,20,23,26,29-decaoxa-32-azahexatriacontan-36-yl)-3,6,9,14,17,22,27,32,35,40,43,46-dodecaazaoctatetracontanedioyl)bis(azanediyl))bis(4-hydroxy-3,1-phenylene))(4R,4′R)-bis(4-((tert-butoxycarbonyl)amino)pentanoate) (100)

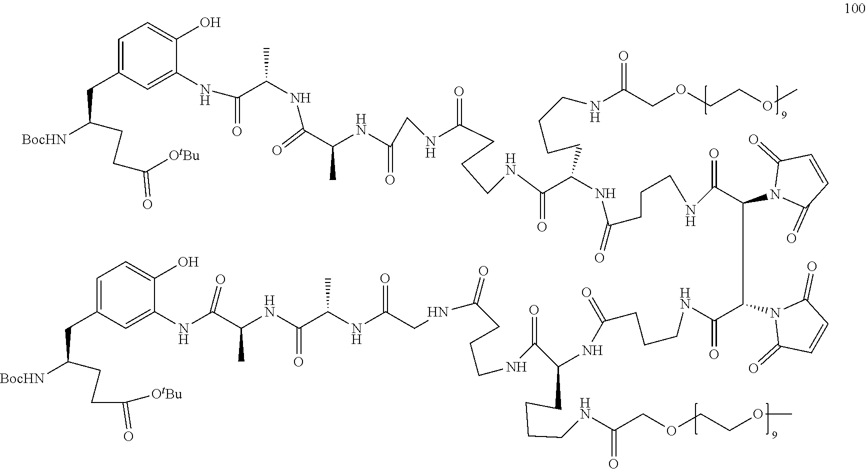

Compound 98 (695 mg, 0.331 mmol) and tert-butyl (R)-5-(3-((S)-2-aminopropanamido)-4-hydroxyphenyl)-4-((tert-butoxycarbonyl)amino)pentanoate (99, 329 mg, 0.729 mmol) were dissolved in dichloromethane (30 mL) and EDCI (190 mg, 0.994 mmol) was added. The reaction was stirred for 1 hour, and then washed with 20 mL of brine, dried over anhydrous sodium sulfate, filtered and concentrated. The residue was purified by preparative HPLC (acetonitrile/water) to give compound 100 as an oil (231 mg, 0.076 mol, 23.52%) MS-ESI (m/z): [M+2H]²⁺ calcd for C₁₃₈H₂₂₆N₂₀O₅₀, 1484.21; found, 1484.44.

### Example 101. Synthesis of (4R,4′R)-5,5′-((((2S,5S,16S,24S,25S,33S,44S,47S)-24,25-bis(2,5-dioxo-2,5-dihydro-1H-pyrrol-1-yl)-2,5,44,47-tetramethyl-4,7,10,15,18,23,26,31,34,39,42,45-dodecaoxo-16,33-bis(31-oxo-2,5,8,11,14,17,20,23,26,29-decaoxa-32-azahexatriacontan-36-yl)-3,6,9,14,17,22,27,32,35,40,43,46-dodecaazaoctatetracontanedioyl)bis(azanediyl))bis(4-hydroxy-3,1-phenylene))bis(4-aminopentanoic acid) (101)

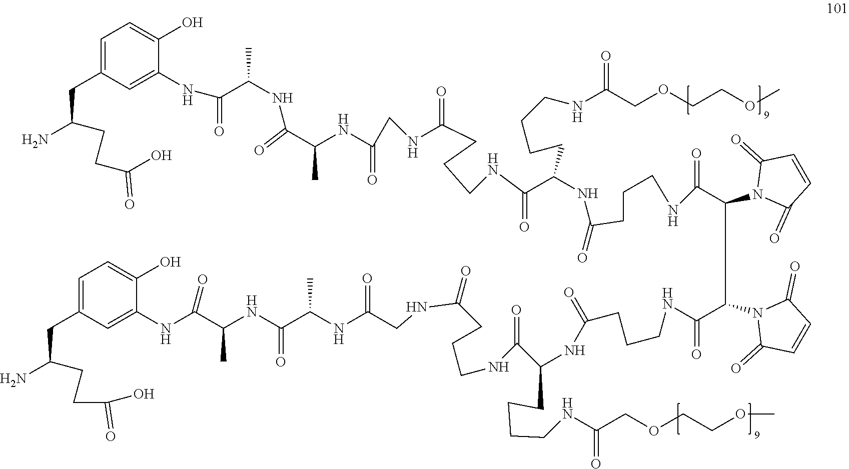

Compound 100 (231 mg, 0.078 mmol) was dissolved in dichloromethane (8 mL) and treated with trifluoroacetic acid (4 mL). The reaction was stirred for 3 hours, and then concentrated, co-evaporated with dichloromethane twice. Compound 101 was obtained as an oil (206 mg, 0.078 mmol, 99%). MS-ESI (m/z): [M+2H]²⁺ calcd for C₁₂₀H₁₉₄N₂₀O₄₆, 1327.98; found, 1327.73.

### Example 102. Synthesis of (4R,4′R)-5,5′-((((2S,5S,16S,24S,25S,33S,44S,47S)-24,25-bis(2,5-dioxo-2,5-dihydro-1H-pyrrol-1-yl)-2,5,44,47-tetramethyl-4,7,10,15,18,23,26,31,34,39,42,45-dodecaoxo-16,33-bis(31-oxo-2,5,8,11,14,17,20,23,26,29-decaoxa-32-azahexatriacontan-36-yl)-3,6,9,14,17,22,27,32,35,40,43,46-dodecaazaoctatetracontanedioyl)bis(azanediyl))bis(4-hydroxy-3,1-phenylene))bis(4-(2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxamido)pentanoic acid) (102)

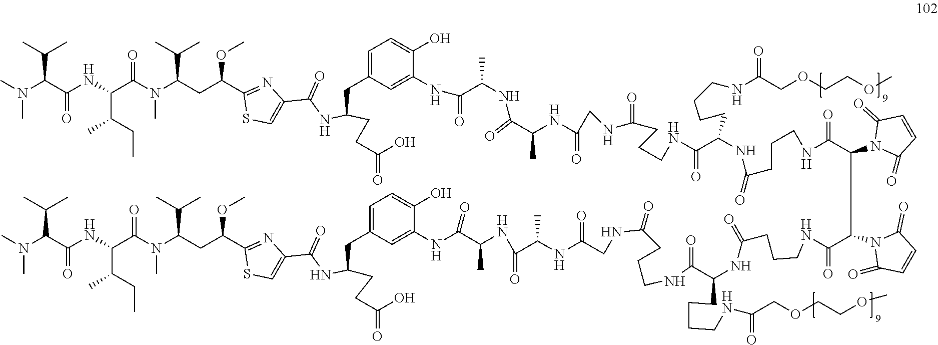

Compound 101 (206 mg, 0.078 mmol) and Tub-2 (131 mg, 0.194 mmol) were dissolved in DMF (2 mL) and then diisopropylethylamine (0.051 mL, 0.311 mmol) was added. The reaction was stirred for 4 hours, neutralized with formic acid, and purified by preparative HPLC (acetonitrile/water, containing 0.1% formic acid) to give compound 102 as a solid (90 mg, 0.02 mmol, 30% yield). MS-ESI (m/z): [M+4H]⁴⁺ calcd for C₁₇₀H₂₇₈N₂₈O₅₄S₂, 911.84; found, 911.67.

### Example 103. Synthesis of 2,5-dioxopyrrolidin-1-yl ((benzyloxy)carbonyl)-L-alaninate (103)

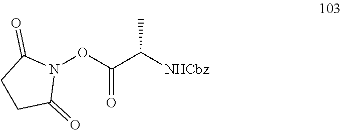 [SMILES: C[C@H](NC(=O)OCc1ccccc1)C(=O)ON1C(=O)CCC1=O]

Cbz-Ala-OH (8.93 g, 40 mmol) was dissolved in dichloromethane (300 mL), NHS (9.20 g, 80 mmol) was added, and after stirring for 5 min, EDCI (23.00 g, 120 mmol) was added in portions. After completion of addition, the reaction was stirred at r.t. for 3 hours, washed with brine, dried over anhydrous sodium sulfate, filtered and concentrated to give a crude product, which was used in the next step without further purification, assuming 100% yield.

### Example 104. Synthesis of (2S,4R)-5-(3-((S)-2-(((benzyloxy)carbonyl)amino) propanamido)-4-hydroxyphenyl)-4-((tert-butoxycarbonyl)amino)-2-methylpentanoic acid (104)

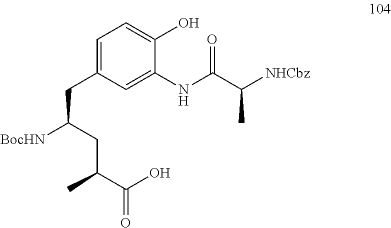 [SMILES: C[C@H](NC(=O)OCc1ccccc1)C(=O)Nc1cc(C[C@@H](C[C@H](C)C(=O)O)NC(=O)OC(C)(C)C)ccc1O]

Compound 103 (crude product from the previous step, 40 mmol) and compound 77 (13.54 g, 40 mmol) were dissolved in tetrahydrofuran (300 mL) and stirred under reflux for 16 hours. The reaction mixture was concentrated under reduced pressure, then diluted with dichloromethane, washed with brine, and dried over anhydrous sodium sulfate, filtered and concentrated. The crude product was purified by silica gel column (MeOH/dichloromethane) to give compound 104 (13.5 g, 62% yield).

### Example 105. Synthesis of (2S,4R)-5-(3-((S)-2-aminopropanamido)-4-hydroxyphenyl)-4-((tert-butoxycarbonyl)amino)-2-methylpentanoic acid (105)

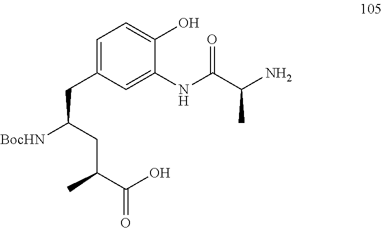 [SMILES: C[C@H](N)C(=O)Nc1cc(C[C@@H](C[C@H](C)C(=O)O)NC(=O)OC(C)(C)C)ccc1O]

Compound 104 (3.70 g, 6.80 mmol) was dissolved in methanol (100 mL) and Pd/C (10% wet, 0.7 g) was added. The reaction flask was evacuated and back-filled with hydrogen for three times, and then stirred under a hydrogen balloon for 6 hours, filtered, concentrated, and co-evaporated with 50 mL of dichloromethane, dried on an oil pump to give compound 105 (2.79 g, 100% yield).

### Example 106. Synthesis of (2S,4R)-5-(3-((S)-2-((S)-2-(((benzyloxy)carbonyl)amino)propanamido)propanamido)-4-hydroxyphenyl)-4-((tert-butoxycarbonyl)amino)-2-methylpentanoic acid (106)

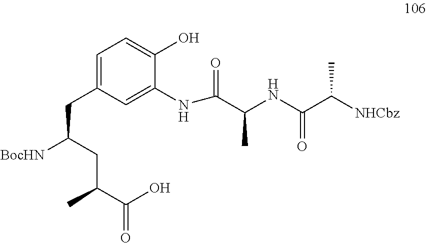 [SMILES: C[C@H](NC(=O)OCc1ccccc1)C(=O)N[C@@H](C)C(=O)Nc1cc(C[C@@H](C[C@H](C)C(=O)O)NC(=O)OC(C)(C)C)ccc1O]

Compound 105 (2.79, 6.8 mmol) and compound 103 (2.18 g, 6.8 mmol) were dissolved in tetrahydrofuran (100 mL) and stirred under reflux for 4 hours. The reaction mixture was concentrated under reduced pressure, then diluted with dichloromethane, washed with brine, and dried over anhydrous sodium sulfate, filtered and concentrated. The crude product was purified by silica gel column (MeOH/dichloromethane) to give compound 106 (2.2 g, 53% yield).

### Example 107. Synthesis of (2S,4R)-5-(3-((S)-2-((S)-2-aminopropanamido)propanamido)-4-hydroxyphenyl)-4-((tert-butoxycarbonyl)amino)-2-methylpentanoic acid (107)

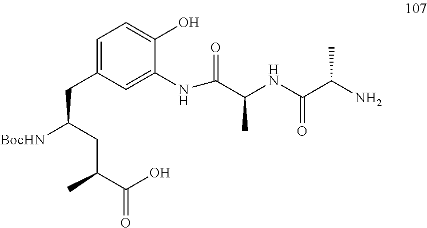 [SMILES: C[C@H](N)C(=O)N[C@@H](C)C(=O)Nc1cc(C[C@@H](C[C@H](C)C(=O)O)NC(=O)OC(C)(C)C)ccc1O]

Compound 106 (1.39 g, 2.26 mmol) was dissolved in methanol (50 mL) and Pd/C (10% wet, 0.3 g) was added. The reaction flask was evacuated and back-filled with hydrogen for three times, and then stirred under a hydrogen balloon for 4 hours, filtered, concentrated, and co-evaporated with 50 mL of dichloromethane, dried on an oil pump to give compound 107 (1.01 g, 93% yield).

### Example 108. Synthesis of tert-butyl ((S)-37-(((benzyloxy)carbonyl)amino)-31-oxo-2,5,8,11,14,17,20,23,26,29-decaoxa-32-azaoctatriacontan-38-oyl)-L-alaninate (108)

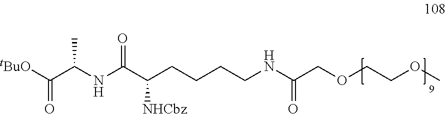

H-Ala-O^{t}Bu·HCl (3.63 g, 2.00 mmol) and compound 20 (1.48 g, 2.40 mmol) were dissolved in THF (30 mL), HATU (1.36 g, 2.40 mmol) and triethylamine (0.33 mL, 2.40 mmol) were added. The reaction was stirred at r.t. for 1 h, concentrated, diluted with 20 mL of water and 25 mL of dichloromethane, the separated organic phase was washed with 5% Na₂CO₃, 1M HCl, dried over anhydrous sodium sulfate, concentrated to dryness and purified by column chromatography to give a colorless liquid (1.51 g, 86% yield). MS-ESI (m/z): [M+H]⁺ calcd for C₄₂H₇₃N₃O₁₆, 876.50; found, 876.50.

### Example 109. Synthesis of tert-butyl ((S)-37-amino-31-oxo-2,5,8,11,14,17,20,23,26,29-decaoxa-32-azaoctatriacontan-38-oyl)-L-alaninate (109)

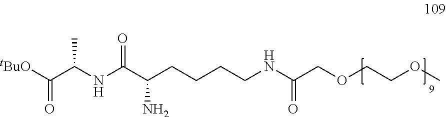

Compound 108 (1.50 g, 1.70 mmol) was dissolved in methanol (80 mL), 10% Pd/C (0.20 g) was added and the reaction flask was evacuated and back-filled with hydrogen for three times, and then stirred under a hydrogen balloon at r.t. for 2 h, filtered, and concentrated to dryness to give an oil (1.26 g, 100% yield). MS-ESI (m/z): [M+H]⁺ calcd for C₃₄H₆₇N₃O₁₄, 742.46; found, 742.50.

### Example 110. Synthesis of di-tert-butyl (2S,5S,13S,14S,22S,25S)-13,14-bis(2,5-dioxo-2,5-dihydro-1H-pyrrol-1-yl)-2,25-dimethyl-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioate (110)

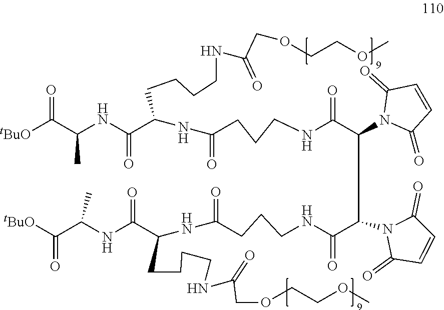

Compound 19 (0.41 g, 0.85 mmol) and compound 109 (1.26 g, 1.70 mmol) were dissolved in THF (10 mL) and DMF (10 mL), HATU (0.78 g, 2.04 mmol) and triethylamine (0.28 mL, 2.04 mmol) were added, the reaction was stirred at r.t. for about 30 min, concentrated, diluted with dichloromethane (40 mL) and washed with 30 mL of brine. The aqueous phase was extracted twice with 100 mL of dichloromethane, the organic phases were combined, dried over anhydrous sodium sulfate, filtered, concentrated to dryness, to give a colorless oil (1.44 g, 88% yield). MS-ESI (m/z): [M+H]⁺ calcd for C₈₈H₁₅₂N₁₀O₃₆, 1926.04; found, 1926.04.

### Example 111. Synthesis of (2S,5S,13S,14S,22S,25S)-13,14-bis(2,5-dioxo-2,5-dihydro-1H-pyrrol-1-yl)-2,25-dimethyl-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioic acid (111)

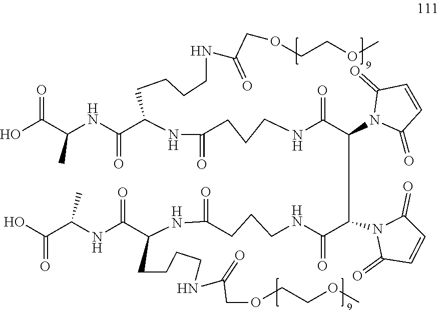

Compound 110 (1.44 g, 0.75 mmol) was dissolved in dichloromethane (10 mL) and formic acid (20 mL), stirred at 55-60° C. for 3 h, concentrated to dryness, and purified by preparative HPLC to give a colorless oil (1.36 g, 100% yield). MS-ESI (m/z): [M+H]⁺ calcd for C₈₀H₁₃₆N₁₀O₃₆, 1813.91; found, 1813.95.

### Example 112. Synthesis of bis(2,5-dioxopyrrolidin-1-yl) (2S,5S,13S,14S,22S,25S)-13,14-bis(2,5-dioxo-2,5-dihydro-1H-pyrrol-1-yl)-2,25-dimethyl-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioate (112)

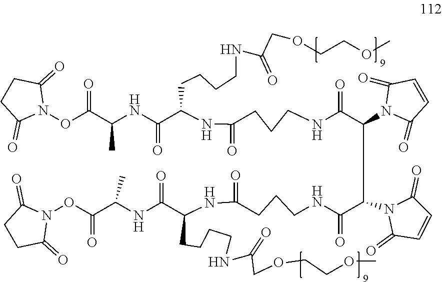

Compound 111 (0.50 g, 0.276 mmol) was dissolved in dichloromethane (50 mL), NHS (0.13 g, 1.103 mmol) was added and stirred for 5 min, and then EDCI (0.32 g, 1.656 mmol) was added under ice-water cooling. The reaction was then warmed to r.t. and stirred for 3 hours, washed with brine, dried over anhydrous sodium sulfate, filtered and concentrated to give a crude product, which was used directly without purification, assuming 100% yield. MS-ESI (m/z): [M+2H]²⁺ calcd for C₈₈H₁₄₂N₁₂O₄₀, 1005.08; found, 1004.80.

### Example 113. Synthesis of (2S,2'S,4R,4′R)-5,5′-((((2S,5S,8S,11S,19S,20S,28S,31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)bis(azanediyl))bis(4-hydroxy-3,1-phenylene))bis(4-((tert-butoxycarbonyl)amino)-2-methylpentanoic acid) (113)

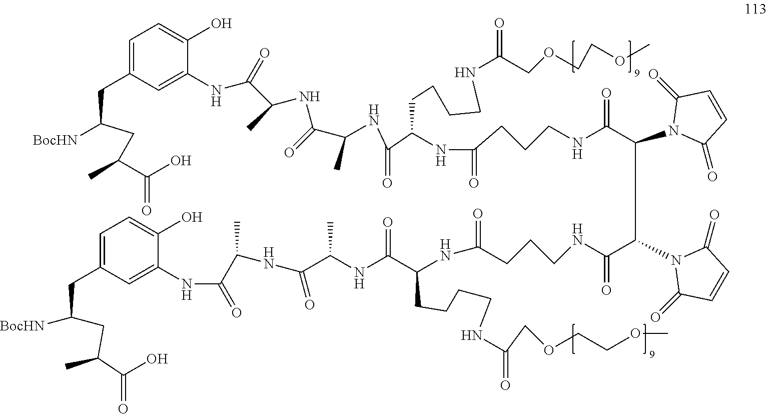

Compound 112 (crude product from the previous step, 0.276 mmol) and compound 107 (0.25 g, 0.521 mmol) were dissolved in tetrahydrofuran (50 mL) and stirred at r.t. for 1 hour. After concentration, the crude product was purified by preparative HPLC (acetonitrile/water) to give compound 113 (268 mg, 35% yield). MS-ESI (m/z): [M+2H]²⁺ calcd for C₁₂₆H₂₀₄N₁₈O₄₈, 1370.55; found, 1370.93.

### Example 114. Synthesis of (2S,2'S,4R,4′R)-5,5′-((((2S,5S,8S,11S,19S,20S,28S,31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)bis(azanediyl))bis(4-hydroxy-3,1-phenylene))bis(4-amino-2-methylpentanoic acid) (114)

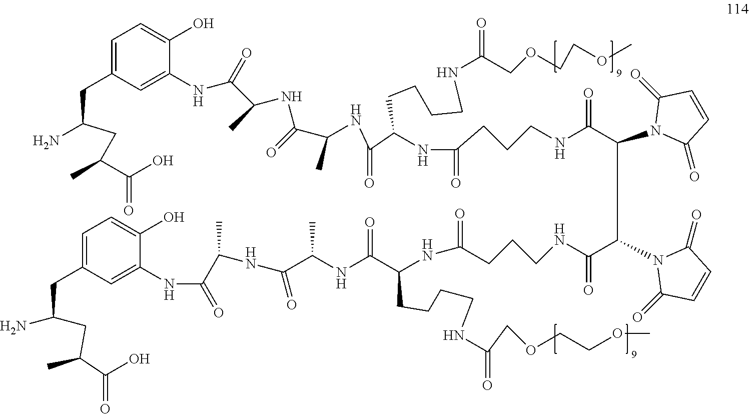

In a 100 mL one-neck reaction flask, compound 113 (268 mg, 0.098 mmol), dichloromethane (12 mL) and trifluoroacetic acid (3 mL) were stirred at r.t. for 1 hour. The reaction mixture was concentrated under reduced pressure, and placed on an oil pump, to give compound 114, which was used without further purification, assuming 100% yield.

### Example 115. Synthesis of (2S,2'S,4R,4′R)-5,5′-((((2S,5S,8S,11S,19S,20S,28S,31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)bis(azanediyl))bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid) (115)

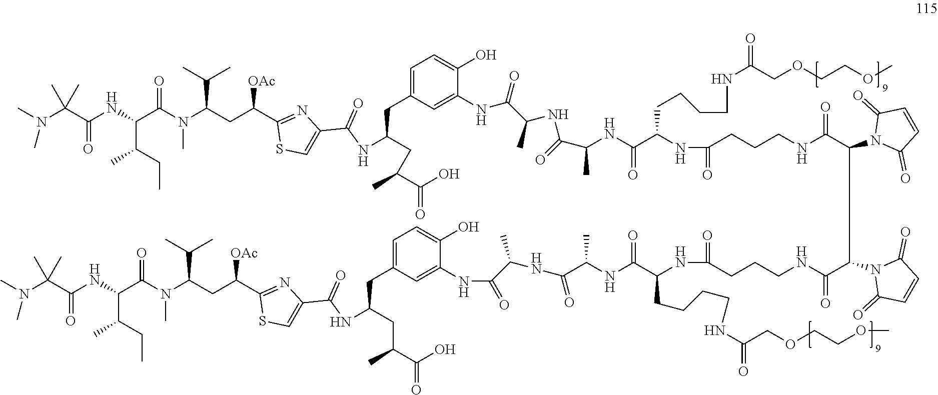

In a 100 mL single-necked reaction flask, to a solution of compound 114 (the crude product from previous step, 0.098 mmol) and Tub-1 (122 mg, 0.176 mmol) in DMF (10 mL), was added dropwise diisopropylethylamine (101 mg, 0.784 mmol). After stirring at r.t. for 5 hours, the reaction was concentrated under reduced pressure. The residue was diluted with 10 mL of dichloromethane, 0.2 mL of formic acid was added dropwise, and the mixture was concentrated, then purified by preparative HPLC (acetonitrile/water), to give the title compound (106 mg, 30% yield). MS-ESI (m/z): [M+2H]²⁺ calcd for C₁₆₆H₂₆₈N₂₆O₅₄S₂, 1779.11; found, 1779.40.

### Example 116. Synthesis of tert-butyl (R)-5-(3-((S)-2-((S)-2-(((benzyloxy)carbonyl)amino)propanamido)propanamido)-4-hydroxyphenyl)-4-((tert-butoxycarbonyl)amino)pentanoate (116)

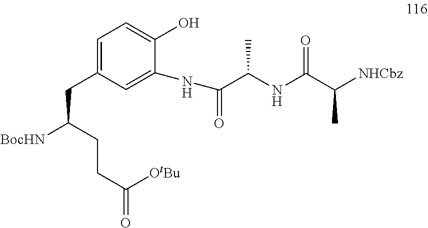 [SMILES: C[C@H](NC(=O)OCc1ccccc1)C(=O)N[C@@H](C)C(=O)Nc1cc(C[C@@H](CCC(=O)OC(C)(C)C)NC(=O)OC(C)(C)C)ccc1O]

To a solution of compound 99 (1.33 g, 2.945 mmol) and Cbz-Ala-OH (0.69 g, 3.093 mmol) in dichloromethane (20 mL), EDCI (1.13 g, 5.891 mmol) was added, the reaction was stirred for 1 hour, washed with brine, dried over anhydrous sodium sulfate, filtered, concentrate and purified by preparative HPLC to give the title compound (1.65 g, 2.512 mmol, 85%). MS-ESI (m/z): [M+H]⁺ calcd for C₃₄H₄₈N₄O₉, 657.34; found 657.31.

### Example 117. Synthesis of tert-butyl (R)-5-(3-((S)-2-((S)-2-aminopropanamido) propanamido)-4-hydroxyphenyl)-4-((tert-butoxycarbonyl)amino)pentanoate (117)

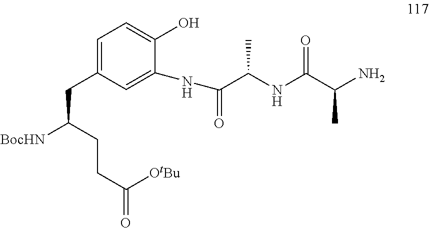 [SMILES: C[C@H](N)C(=O)N[C@@H](C)C(=O)Nc1cc(C[C@@H](CCC(=O)OC(C)(C)C)NC(=O)OC(C)(C)C)ccc1O]

To a solution of compound 116 (1.65 g, 0.003 mol) in methanol (20 mL), 10% Pd/C (0.27 g, 0.003 mol) was added, and the reaction flask was evacuated and back-filled with hydrogen for three times, and then stirred under a hydrogen balloon at r.t. for 2 h, filtered, and concentrated to dryness to afford compound 117 (1.24 g, 95% yield). MS-ESI (m/z): [M+H]⁺ calcd for C₂₆H₄₂N₄O₇, 523.31; found 523.29.

### Example 118. Synthesis of di-tert-butyl 5,5′-((((2S,5S,8S,11S,19S,20S,28S,31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22, 27,30,33,36-decaazaoctatriacontanedioyl)bis(azanediyl))bis(4-hydroxy-3,1-phenylene)) (4R,4′R)-bis(4-((tert-butoxycarbonyl)amino)pentanoate) (118)

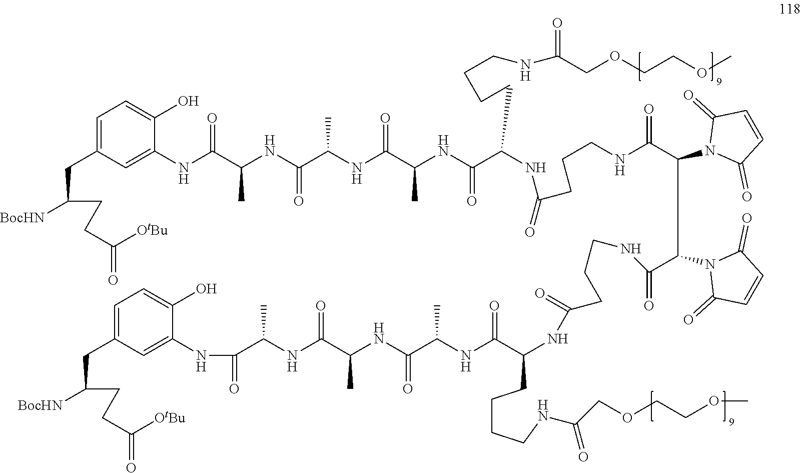

Compound 112 (0.34 g, 0.647 mmol) and compound 117 (0.65 g, 0.324 mmol) were dissolved in THF (15 mL). The reaction mixture was stirred for 1 hour, concentrated and purified by preparative HPLC to give compound 118 (0.25 g, 27% yield). MS-ESI (m/z): [M+2H]²⁺ calcd for C₁₃₂H₂₁₆N₁₈O₄₈, 1411.75; found 1412.88.

### Example 119. Synthesis of (4R,4′R)-5,5′-((((2S,5S,8S,11S,19S,20S,28S, 31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioyl)-bis(azanediyl))bis(4-hydroxy-3,1-phenylene))bis(4-aminopentanoic acid) (119)

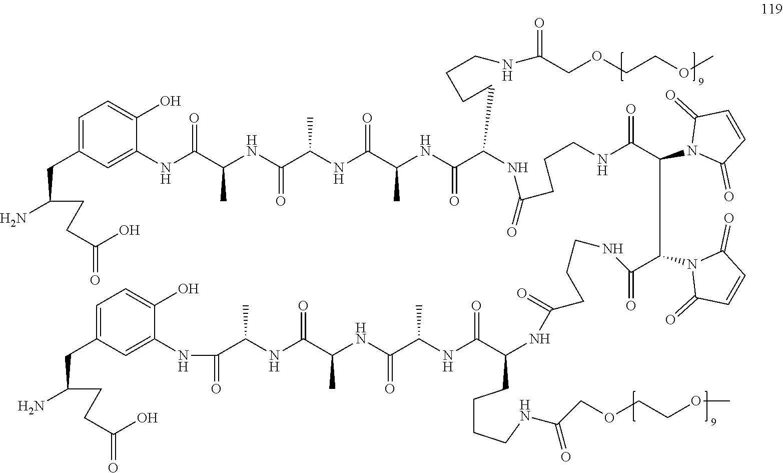

Compound 118 (0.25 g, 0.089 mmol) was dissolved in dichloromethane (8 mL) and trifluoroacetic acid (8 mL). After stirring for 1 hour, the reaction solution was concentrated to give compound 119 (0.413 g, >100% yield). MS-ESI (m/z): [M+2H]²⁺ calcd for C₁₁₄H₁₈₄N₁₈O₄₄, 1255.63; found 1256.01.

### Example 120. Synthesis of (4R,4′R)-5,5′-((((2S,5S,8S,11S,19S,20S,28S, 31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27, 30,33,36-decaazaoctatriacontanedioyl)bis(azanediyl))bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)pentanoic acid) (120)

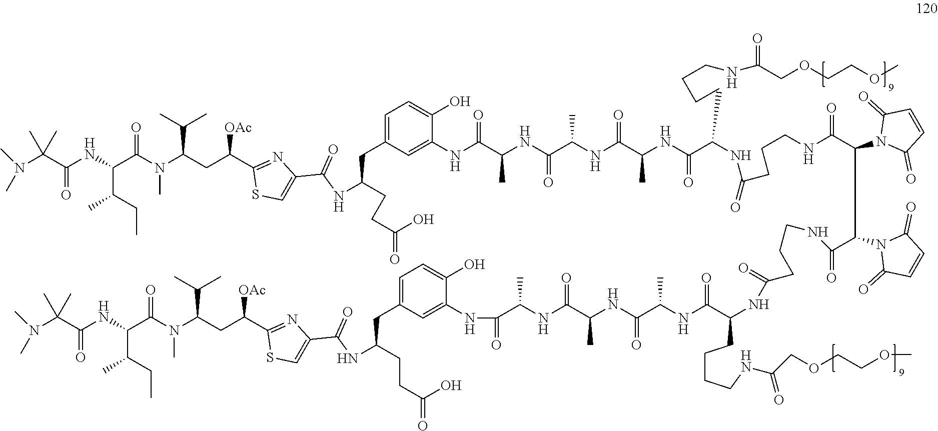

Compound 119 (0.12 g, 0.175 mmol) and Tub-1 (0.22 g, 0.088 mmol) were dissolved in DMF (2 mL), and diisopropylethylamine (0.116 mL, 0.701 mmol) was added dropwise. The reaction was stirred for 2 hours and purified by preparative HPLC to give compound 120 (0.119 g, 38% yield). MS-ESI (m/z): [M+2H]²⁺ calcd for C₁₆₄H₂₆₄N₂₆O₅₄S₂, 1763.91; found 1765.30.

### Example 121. Synthesis of (2S,2'S,4R,4′R)-5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis (azanediyl))bis(4-hydroxy-3,1-phenylene))bis(4-(2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxamido)-2-methylpentanoic acid) (121)

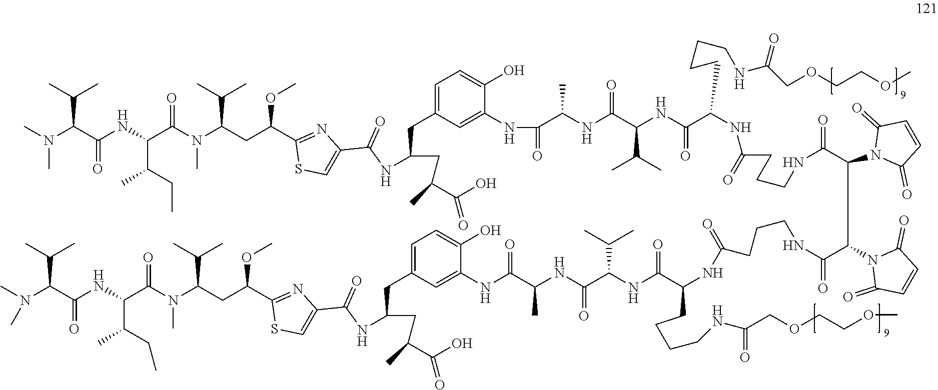

Compound 82 (250 mg, 0.102 mmol) and Tub-2 (207 mg, 0.306 mmol) were dissolved in DMF (2 mL), and diisopropylethylamine (0.034 mL, 0.204 mmol) was added. The reaction was stirred for 6 hours and purified by preparative HPLC to give compound 121 (180 mg, 5100 yield). ESI-MS (m/z): [M+2H]²⁺ calcd for C₁₆₄H₂₇₀N₂₄O₅₀S₂, 1720.94; found 1721.94.

### Example 122. Synthesis of tert-butyl (R)-5-(3-((37S,40S,43S)-37-(((benzyloxy)carbonyl)amino)-40-isopropyl-43-methyl-31,38,41-trioxo-2,5,8,11,14,17,20,23,26,29-decaoxa-32,39,42-triazatetratetracontan-44-amido)-4-hydroxyphenyl)-4-((tert-butoxycarbonyl)amino)pentanoate (122)

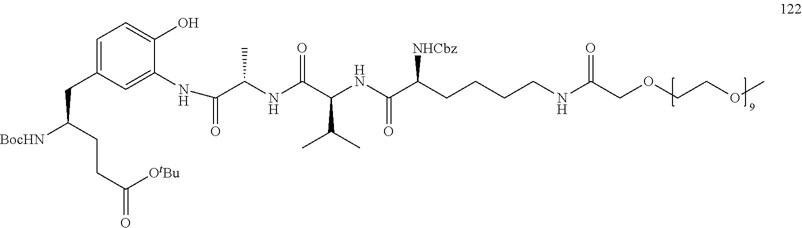

Compound 76 (6.0 g, 5.9 mmol) and compound 69 (2.7 g, 7.1 mmol) were dissolved in THF (100 mL) and heated to 60° C. for 20 hours. The reaction was concentrated, and the residue was purified by silica gel column (dichloromethane:MeOH=20:1) to afford 7.1 g of the desired product as a gray solid (93% yield).

### Example 123. Synthesis of tert-butyl (R)-5-(3-((37S,40S,43S)-37-amino-40-isopropyl-43-methyl-31,38,41-trioxo-2,5,8,11,14,17,20,23,26,29-decaoxa-32,39,42-triazatetratetracontan-44-amido)-4-hydroxyphenyl)-4-((tert-butoxycarbonyl)amino)pentanoate (123)

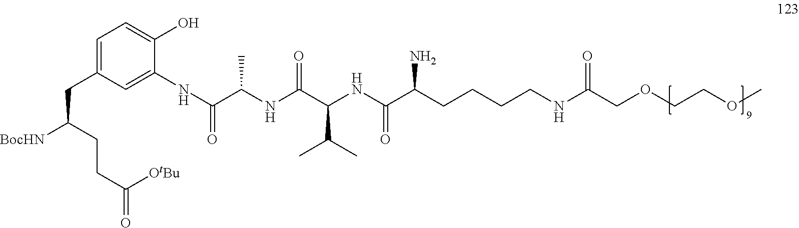

Compound 122 (7.1 g, 5.7 mmol) was dissolved in isopropanol (80 mL), and Pd/C (10% wet, 0.8 g) was added. The reaction flask was evacuated and back-filled with hydrogen for three times, and then stirred at 50° C. under a hydrogen balloon for 2.5 hours, filtered, concentrated, and dried on an oil pump to give a gray solid (5.9 g, 93% yield).

### Example 124. Synthesis of di-tert-butyl 5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis (azanediyl))bis(4-hydroxy-3,1-phenylene))(4R,4′R)-bis(4-((tert-butoxycarbonyl) amino)pentanoate) (124)

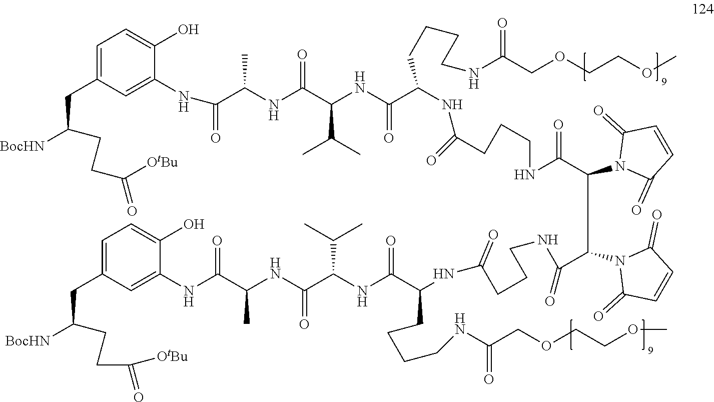

Compound 80 (510 mg, 0.7 mmol) and compound 123 (1900 mg, 1.7 mmol) were dissolved in DMF (20 mL) and cooled to 0° C. N-methylmorpholine (190 mg, 1.9 mmol) was added, and the reaction was continued at 0° C. for 3 hours. The reaction solution was diluted with dichloromethane (50 mL) and washed with brine (4×60 mL). The organic phase was dried with anhydrous sodium sulfate, filtered, and the filtrate was concentrated, purified by silica gel column (dichloromethane:MeOH=20:1˜6:1), and 1.2 g of the desired product was obtained as a brown-grey oil (58% yield). ESI-MS (m/z): [M+2H]²⁺ calcd for C₁₃₀H₂₁₄N₁₆O₄₆, 1368.75; found 1369.34.

### Example 125. Synthesis of (4R,4′R)-5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis (azanediyl))bis(4-hydroxy-3,1-phenylene))bis(4-aminopentanoic acid) (125)

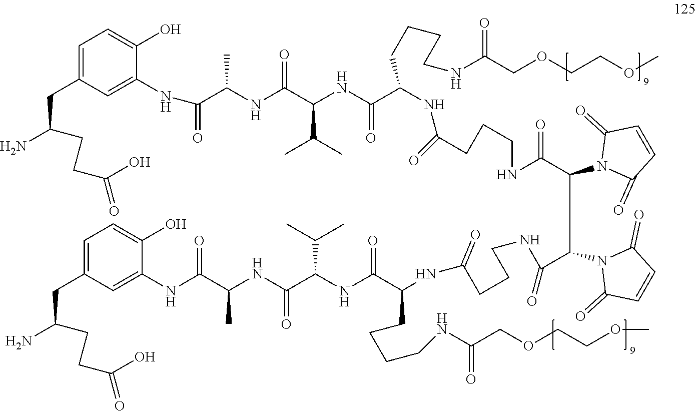

Compound 124 (500 mg, 0.18 mmol) was dissolved in dichloromethane (5 mL), and treated with trifluoroacetic acid (5 mL) for 2 hours. The reaction solution was concentrated to afford 600 mg of crude product as an orange-red oil. ESI-MS (m/z): [M+2H]²⁺ calcd for C₁₁₂H₁₈₂N₁₆O₄₂, 1213.63; found 1213.83.

### Example 126. Synthesis of (4R,4′R)-5,5′-((((2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioyl)bis(azanediyl)) bis(4-hydroxy-3,1-phenylene))bis(4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)pentanoic acid) (126)

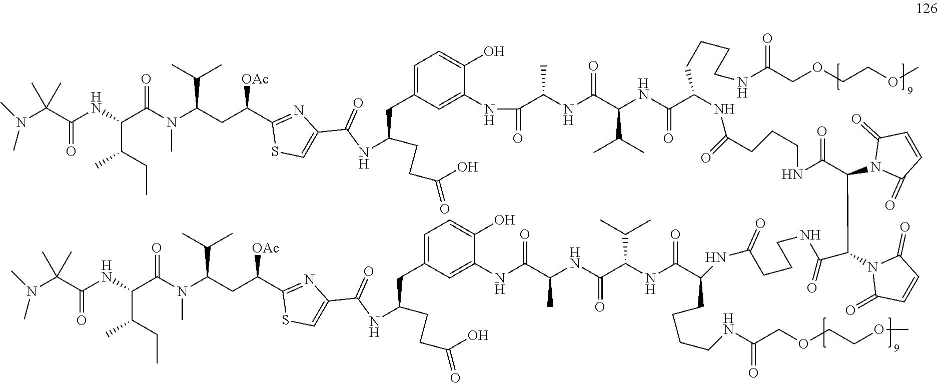

Compound 125 (440 mg, 0.18 mmol) and Tub-1 (320 mg, 0.45 mmol) were dissolved in DMF (5 mL), diisopropylethylamine (70 mg, 0.54 mmol) was added at 0° C. and stirred, and the reaction was continued at 0° C. for 3 hours. The reaction solution was concentrated and the residue was purified by preparative HPLC to afford 92 mg of the desired product as a white solid (15% yield). ESI-MS (m/z): [M+2H]²⁺ calcd for C₁₆₂H₂₆₂N₂₄O₅₂S, 1720.90; found 1721.66.

### Example 127. Synthesis of tert-butyl ((S)-35-amino-24-methyl-1-(11-oxidaneyl)-29-oxo-3,6,9,12,15,18,21,2413,27-nonaoxa-30-azahexatriacontan-36-oyl)-L-valyl-L-alaninate (127)

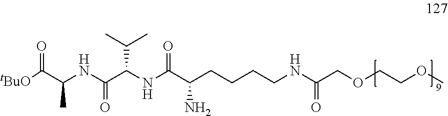

Compound 74 (15 g, 15.4 mmol) was dissolved in isopropyl alcohol (150 mL), palladium on carbon (10 wt %, 2.0 g) was added, and the reaction flask was evacuated and back-filled with hydrogen for three times. After stirring for 2 days, the reaction mixture was filtered and the filtrate was concentrated to give the title compound 127 (12 g, 92% yield). MS-ESI (m/z): [M+H]⁺ calcd for C₃₉H₇₇N₄O₁₅, 841.53; found 841.53.

### Example 128. Synthesis of di-tert-butyl (2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioate (128)

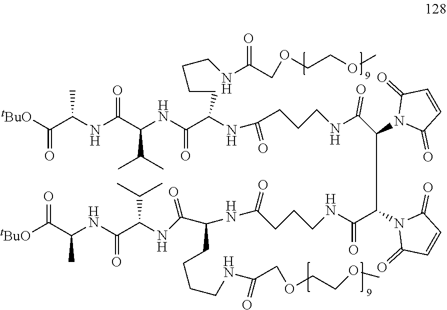

To a solution of compound 127 (5.5 g, 6.6 mmol) and compound 19 (1.5 g, 3.1 mmol) in DMF (100 mL), were added HATU (4.8 g, 12.5 mmol) and NMM (1.3 g, 12.5 mmol). The reaction was stirred at r.t. until complete conversion, and then concentrated under vacuum and poured into water (200 mL), extracted with dichloromethane (3×100 mL). The combined organic phases were washed with water (50 mL) and brine (50 mL), dried over sodium sulfate, filtered and concentrated to give compound 128 (6.5 g, 100% yield). MS-ESI (m/z): [M+2H]²⁺ calcd for C₉₈H₁₇₁N₁₂O₃₈, 1063.08; found 1063.78.

### Example 129. Synthesis of (2S,5S,8S,16S,17S,25S,28S,31S)-16,17-bis(2,5-dioxo-2,5-dihydro-1H-pyrrol-1-yl)-5,28-diisopropyl-2,31-dimethyl-4,7,10,15,18,23,26,29-octaoxo-8,25-bis(31-oxo-2,5,8,11,14,17,20,23,26,29-decaoxa-32-azahexatriacontan-36-yl)-3,6,9,14,19,24,27,30-octaazadotriacontanedioic acid (129)

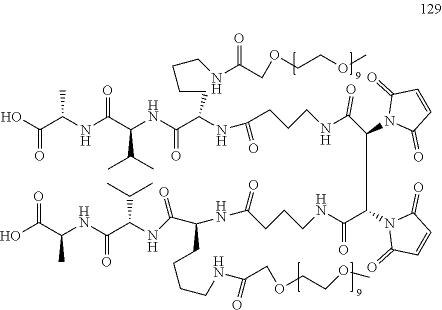

Compound 128 (6.5 g, 3.1 mmol) was dissolved in dichloromethane (50 mL) and trifluoroacetic acid (50 mL). The mixture was stirred for 2 hours, concentrated under vacuum and purified by preparative HPLC to give product 129 (3.1 g, 50% yield). MS-ESI (m/z): [M+2H]²⁺ calcd for C₉₀H₁₅₅N₁₂O₃₈, 1006.03; found 1006.13.

### Example 130. Synthesis of (2S,3S)-2,3-bis(2,5-dioxo-2,5-dihydro-1H-pyrrol-1-yl)-N1,N4-bis((S)-37-(((S)-1-(((S)-1-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-1-oxopropan-2-yl)amino)-3-methyl-1-oxobutan-2-yl)carbamoyl)-31,39-dioxo-2,5,8,11,14,17,20,23,26,29-decaoxa-32,38-diazadotetracontan-42-yl)succinamide (130)

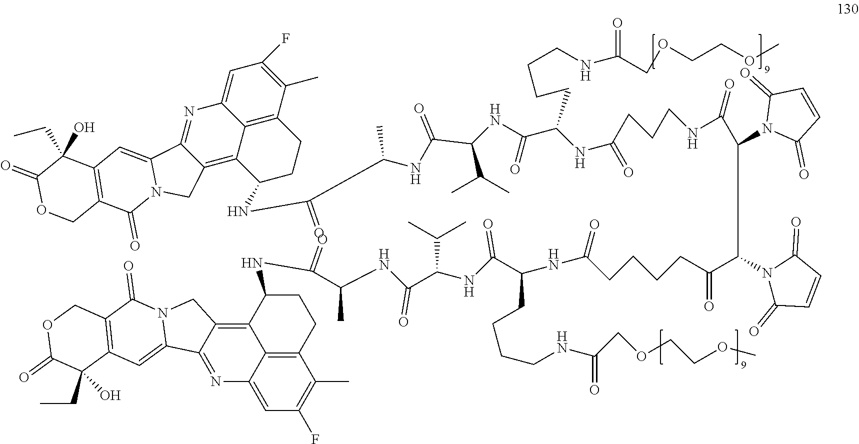

To a solution of compound 129 (280 mg, 0.139 mmol) and exatecan mesylate (148 mg, 0.278 mmol) in DMF (5 mL), were added HATU (158 mg, 0.417 mmol) and diisopropylethylamine (92 L, 0.557 mmol). The reaction was stirred at r.t. until complete conversion, and then concentrated and purified by preparative HPLC to give product 130 (101 mg, 25% yield). MS-ESI (m/z): calcd for C₁₃₈H₁₉₅F₂N₁₈O₄₄ [M+2H]²⁺: 1424.18, found 1425.00.

### Example 131. Synthesis of tert-butyl ((benzyloxy)carbonyl)-L-alanyl-L-alanyl-L-alaninate (131)

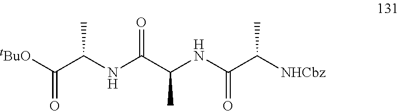 [SMILES: C[C@H](NC(=O)OCc1ccccc1)C(=O)N[C@@H](C)C(=O)N[C@@H](C)C(=O)OC(C)(C)C]

Compound 61 (4.0 g, 18.49 mmol) and Cbz-Ala-OH (4.1 g, 18.49 mmol) was dissolved in dichloromethane (100 mL), to which EDCI (7.0 g, 36.51 mmol) and diisopropylethylamine (4.7 g, 36.36 mmol) were added at 5° C. After stirring for about 0.5 h, the reaction was washed by water and brine, purified by a silica gel column, eluted with dichloromethane and methanol to give compound 131 as a white solid (1.6 g, 20% yield). MS-ESI (m/z): [M+H]⁺ calcd for C₂₁H₃₂N₃O₆, 422.22; found 422.22.

### Example 132. Synthesis of tert-butyl L-alanyl-L-alanyl-L-alaninate (132)

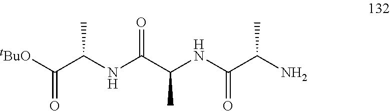 [SMILES: C[C@H](N)C(=O)N[C@@H](C)C(=O)N[C@@H](C)C(=O)OC(C)(C)C]

Compound 131 (1.6 g, 3.79 mmol) was dissolved in methanol (100 mL), 10% Pd/C (0.16 g) was added, the reaction flask was evacuated and back-filled with hydrogen for three times, and then stirred under a hydrogen balloon at r.t. for 1 h, filtered, concentrated to give compound 132 as colorless liquid (1.1 g, 100% yield). MS-ESI (m/z): [M+H]⁺ calcd for C₁₃H₂₆N₃O₄, 288.18; found 288.18.

### Example 133. Synthesis of tert-butyl ((S)-37-(((benzyloxy)carbonyl)amino)-31-oxo-2,5,8,11,14,17,20,23,26,29-decaoxa-32-azaoctatriacontan-38-oyl)-L-alanyl-L-alanyl-L-alaninate (133)

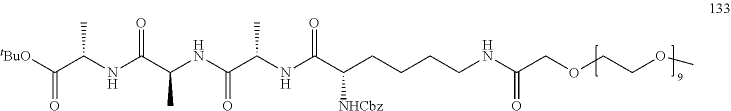

Compound 132 (1.1 g, 3.83 mmol) and compound 20 (2.6 g, 3.47 mmol) were dissolved in THF (50 mL), HATU (2.0 g, 5.25 mmol) and diisopropylethylamine (0.9 g, 6.94 mmol) were added at 5° C. After stirring for about 0.5 h, the reaction was concentrated, diluted with dichloromethane, washed with water (100 mL), 5% Na₂CO₃ (80 mL), 1M HCl (80 mL), filtered and concentrated. The residue was purified by a silica gel column, eluted with dichloromethane and methanol to give compound 133 as white solid (2.9 g, 81% yield). MS-ESI (m/z): [M+H]⁺ calcd for C₄₈H₈₄N₅O₁₈, 1018.57; found 1018.57.

### Example 134. Synthesis of tert-butyl ((S)-37-amino-31-oxo-2,5,8,11,14,17,20,23,26,29-decaoxa-32-azaoctatriacontan-38-oyl)-L-alanyl-L-alanyl-L-alaninate (134)

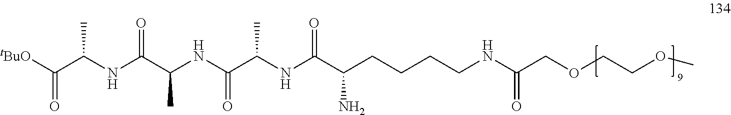

Compound 133 (2.9 g, 2.84 mmol) was dissolved in methanol (150 mL), and 10% Pd/C (0.29 g) was added. The reaction flask was evacuated and back-filled with hydrogen for three times, and then stirred under a hydrogen balloon at r.t. for 1 h, filtered, concentrated to give compound 134 as colorless oil (2.5 g, 100% yield). MS-ESI (m/z): [M+H]⁺ calcd for C₄₀H₇₈N₅O₁₆, 884.54; found 884.54.

### Example 135. Synthesis of di-tert-butyl (2S,5S,8S,11S,19S,20S,28S,31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioate (135)

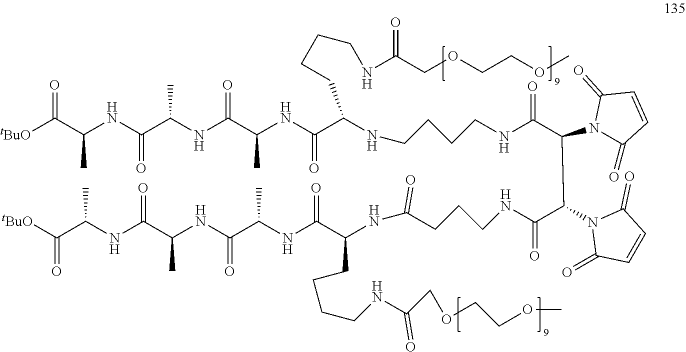

Compound 134 (0.83 g, 0.94 mmol) and compound 19 (204.3 mg, 0.43 mmol) were dissolved in mixed solvents of THF (10 mL) and DMF (10 mL). HATU (470 mg, 1.24 mmol) and diisopropylethylamine (216 mg, 1.67 mmol) were added. After stirring for 1 h at r.t., the reaction was concentrated, diluted with 100 mL of dichloromethane, washed with brine (50 mL), concentrated to give compound 135 as a colorless oil (0.924 g, 100% yield). MS-ESI (m/z): [M+2H]²⁺ calcd for C₁₀₀H₁₇₃N₁₄O₄₀, 1106.04; found 1106.25.

### Example 136. Synthesis of (2S,5S,8S,11S,19S,20S,28S,31S,34S,37S)-19,20-bis(2,5-dioxo-2,5-dihydro-1H-pyrrol-1-yl)-2,5,8,31,34,37-hexamethyl-4,7,10,13,18,21,26,29, 32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioic acid (136)

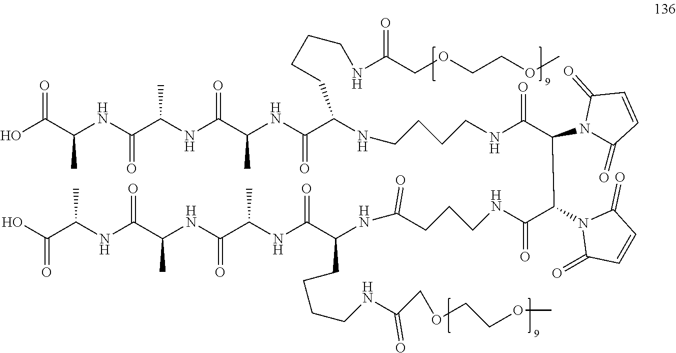

Compound 135 (924.1 mg, 0.42 mmol) was dissolved in 10 mL of dichloromethane and 20 mL of formic acid. After stirring for 3 h at 55-60° C., the reaction was concentrated, and the residue was purified by preparative HPLC to give compound 136 as a white solid (0.42 g, 48% yield). MS-ESI (m/z): [M+H]⁺ calcd for C₉₂H₁₅₇N₁₄O₄₀, 1050.03; found 1050.03.

### Example 137. Synthesis of (2S,3S)-2,3-bis(2,5-dioxo-2,5-dihydro-1H-pyrrol-1-yl)-N1,N4-bis((S)-37-(((S)-1-(((S)-1-(((S)-1-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)carbamoyl)-31,39-dioxo-2,5,8,11,14,17,20,23,26,29-decaoxa-32,38-diazadotetracontan-42-yl)succinamide (137)

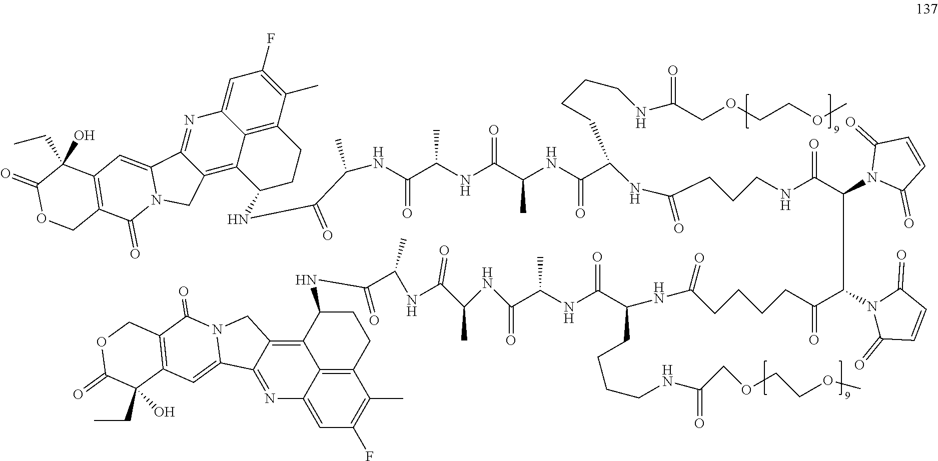

Compound 136 (202.5 mg, 0.09 mmol) and exatecan (111.1 mg, 0.21 mmol) were dissolved in 10 mL of DMF, and HATU (110.0 mg, 0.29 mmol) and diisopropylethylamine (50.0 mg, 0.38 mmol) were added. The reaction was stirred at r.t. for about 30 min, concentrated, and purified by preparative HPLC to give compound 137 as a white solid (176.0 mg, 62% yield). MS-ESI (m/z): [M+H]⁺ calcd for C₁₄₀H₁₉₇F₂N₂₀O₄₆, 1467.18; found 1468.62.

### Example 138. Synthesis of di-tert-butyl (2S,5S,8S,11S,28S,31S,34S,37S)-19,20-bis(2,5-dioxo-2,5-dihydro-1H-pyrrol-1-yl)-2,5,8,31,34,37-hexamethyl-4,7,10,13,18, 21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioate (138)

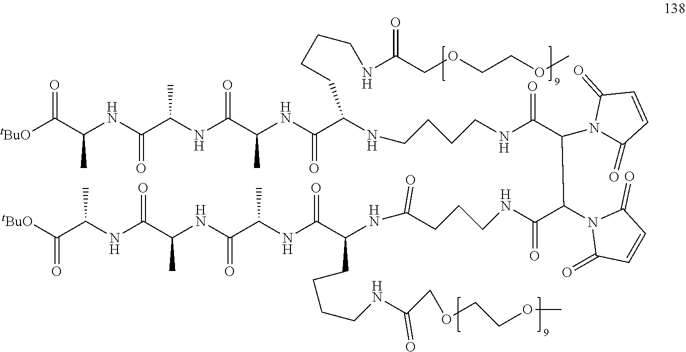

To a solution of compound 134 (4.07 g, 4.60 mmol) and compound 12 (1.00 g, 2.09 mmol) in DMF (40 ml), were added HATU (2.38 g, 6.27 mmol) and diisopropylethylamine (0.69 mL, 4.18 mmol). The reaction was stirred at r.t. until complete conversion, and then concentrated under vacuum and diluted with water (200 mL), extracted with dichloromethane (3×100 mL). The combined organic phases were washed with water (50 mL) and brine (50 mL), dried over sodium sulfate, filtered and concentrated to give product 138 (4.6 g, 100% yield). MS-ESI (m/z): [M+H]⁺ calcd for C₁₀₀H₁₇₃N₁₄O₄₀, 2210.19; found 2210.19.

### Example 139. Synthesis of (2S,5S,8S,11S,28S,31S,34S,37S)-19,20-bis(2,5-dioxo-2,5-dihydro-1H-pyrrol-1-yl)-2,5,8,31,34,37-hexamethyl-4,7,10,13,18,21,26,29,32,35-decaoxo-11,28-bis(31-oxo-2,5,8,11,14,17,20,23,26,29-decaoxa-32-azahexatriacontan-36-yl)-3,6,9,12,17,22,27,30,33,36-decaazaoctatriacontanedioic acid (139)

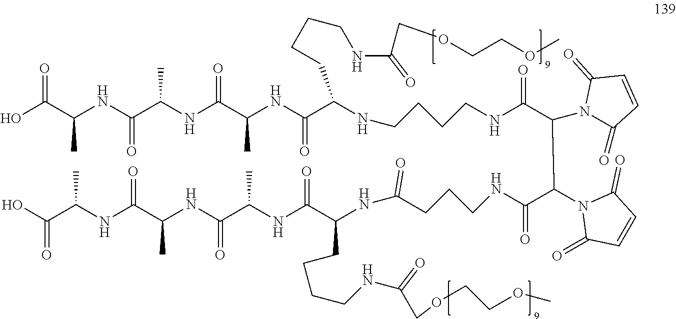

Compound 138 (4.6 g, 1.2 mmol) was dissolved in formic acid (40 mL) and dichloromethane (20 mL). The mixture was heated to 60° C. and stirred for 3 hours, and then concentrated under vacuum and purified by preparative HPLC to give product 139 (2.7 g, 61% yield). MS-ESI (m/z): [M+H]⁺ calcd for C₉₂H₁₅₈N₁₄O₄₀, 2098.06; found 2098.06.

### Example 140. Synthesis of 2,3-bis(2,5-dioxo-2,5-dihydro-1H-pyrrol-1-yl)-N1-((S)-31,39-dioxo-37-(((S)-1-oxo-1-(((S)-1-oxo-1-(((S)-3-oxobutan-2-yl)amino)propan-2-yl)amino)propan-2-yl)carbamoyl)-2,5,8,11,14,17,20,23,26,29-decaoxa-32,38-diazadotetracontan-42-yl)-N4-((S)-37-(((S)-1-(((S)-1-(((S)-1-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)carbamoyl)-31,39-dioxo-2,5,8,11,14,17,20,23,26,29-decaoxa-32,38-diazadotetracontan-42-yl)succinamide (140)

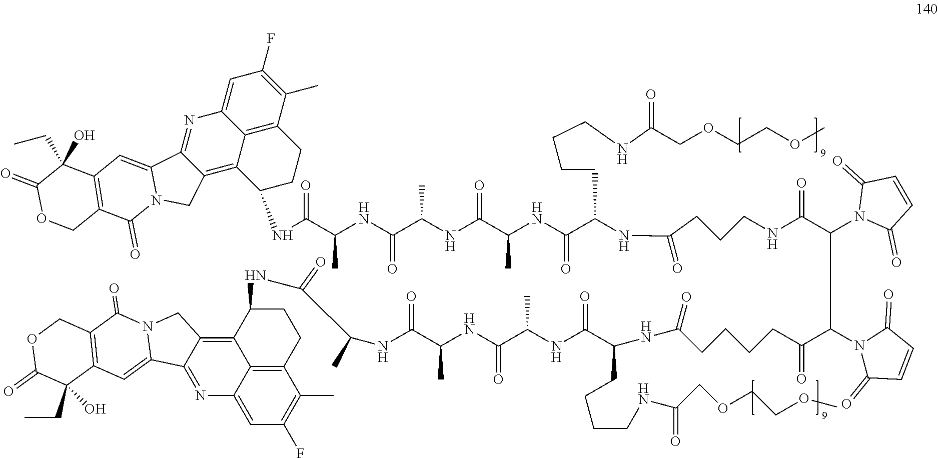

To a solution of compound 139 (300 mg, 0.143 mmol) and exatecan mesylate (152 mg, 0.286 mmol) in DMF (10 mL), were added HATU (163 mg, 0.429 mmol) and diisopropylethylamine (94 μL, 0.572 mmol). The reaction was stirred at r.t. until complete conversion, and then concentrated and purified by preparative HPLC to give product 140 (263 mg, 62% yield). MS-ESI (m/z): [M+H]⁺ calcd for C₁₄₀H₁₉₇F₂N₂₀O₄₆, 2932.36; found 2932.36.

### Example 141. Synthesis of tert-butyl ((S)-37-(((benzyloxy)carbonyl)amino)-31-oxo-2,5,8,11,14,17,20,23,26,29-decaoxa-32-azaoctatriacontan-38-oyl)-L-valinate (141)

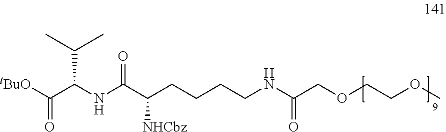

To a solution of compound 20 (10.0 g, 13.4 mmol) and H-Val-O^{t}Bu·HCl (2.40 g, 13.8 mmol) in THF (100 mL), HATU (7.60 g, 20.0 mmol) and diisopropylethylamine (4.4 mL, 26.7 mmol) were added. The reaction was stirred at r.t. until completion, as indicated by LC-MS. The solvent was removed and the residue was diluted with dichloromethane (200 mL), washed with water (50 mL), saturated sodium bicarbonate (50 mL), 2 N HCl (50 mL), and brine (50 mL), dried over sodium sulfate, filtered and concentrated to give compound 141 (12.0 g, 100% yield). MS-ESI (m/z): [M+H]⁺ calcd for C₄₄H₇₈N₃O₁₆, 904.53; found 904.53.

### Example 142. Synthesis of tert-butyl ((S)-37-amino-31-oxo-2,5,8,11,14,17, 20,23,26,29-decaoxa-32-azaoctatriacontan-38-oyl)-L-valinate (142)

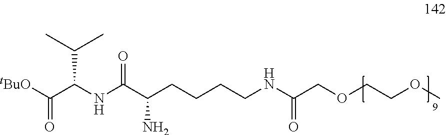

Compound 141 (10.0 g, 11.1 mmol) was dissolved in THF (100 mL), 10% palladium on carbon (1.0 g) was added, and the reaction flask was evacuated and back-filled with hydrogen for three times. After stirring for 2 hours, the reaction mixture was filtered and the filtrate was concentrated to give the title compound 142 (8.6 g, 100% yield). MS-ESI (m/z): [M+H]⁺ calcd for C₃₆H₇₂N₃O₁₄, 770.49; found 770.49.

### Example 143. Synthesis of di-tert-butyl (2S,5S,13S,14S,22S,25S)-13,14-bis(2,5-dioxo-2,5-dihydro-1H-pyrrol-1-yl)-2,25-diisopropyl-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioate (143)

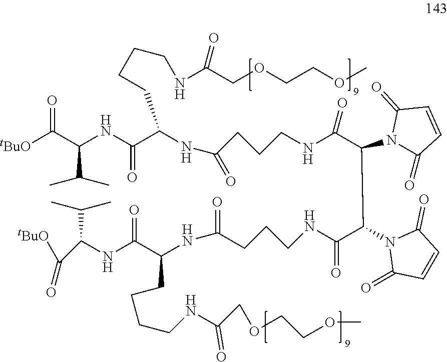

To a solution of compound 19 (1.20 g, 2.5 mmol) and compound 142 (4.10 g, 5.5 mmol) in mixed solvents of THF (50 mL) and DMF (10 mL), HATU (2.80 g, 7.5 mmol) and diisopropylethylamine (1.0 mL, 7.5 mmol) were added. The reaction was stirred at r.t. until completion, as indicated by LC-MS. The solvent was removed and the residue was dissolved in dichloromethane (200 mL), washed with water (50 mL), saturated sodium bicarbonate (50 mL), 2 N HCl (50 mL), and brine (50 mL), dried over sodium sulfate, filtered and concentrated to give the title compound (4.8 g, 100% yield). MS-ESI (m/z): [M+H]⁺ calcd for C₉₂H₁₆₁N₁₀O₃₆, 1982.10; found 1982.10.

### Example 144. Synthesis of (2S,5S,13S,14S,22S,25S)-13,14-bis(2,5-dioxo-2,5-dihydro-1H-pyrrol-1-yl)-2,25-diisopropyl-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioic acid (144)

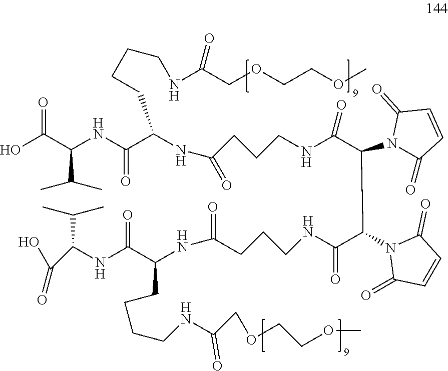

Compound 143 (4.8 g, 2.5 mmol) was dissolved in formic acid (40 mL) and dichloromethane (20 mL), and then heated to 60° C. The reaction was stirred until completion, as indicated by LC-MS and then concentrated to give the title compound (2.2 g, 48% yield). MS-ESI (m/z): [M+H]⁺ calcd for C₈₄H₁₄₅N₁₀O₃₆, 1869.97; found 1869.97.

### Example 145. Synthesis of (2-((((9H-fluoren-9-yl)methoxy)carbonyl)amino) acetamido)methyl acetate (146)

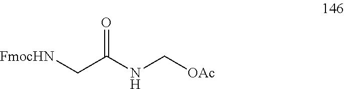

To a solution of Fmoc-Gly-Gly-OH (7.3 g, 20.6 mmol) in tetrahydrofuran (100 mL) and toluene (30 mL), pyridine (2 mL, 24.8 mmol) was added, followed by lead tetraacetate (11 g, 24.8 mmol) under N₂. The reaction mixture was heated to reflux for 5 hours, cooled to r.t., and then filtered. The filtrate was concentrated, diluted with ethyl acetate (300 mL) and water (50 mL). The separated organic phase was washed with brine (50 mL), dried over sodium sulfate, filtered, concentrated. The residue was purified by silica gel column, eluted with petroleum ether/ethyl acetate to give a white solid (7.1 g, 76% yield). MS-ESI (m/z): [M+H]⁺ calcd for C₂₀H₂₀N₂O₅, 369.14; found 369.14.

### Example 146. Synthesis of benzyl (2-hydroxyacetyl)-L-alaninate (147)

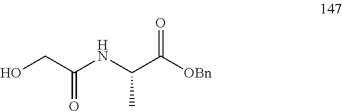 [SMILES: C[C@H](NC(=O)CO)C(=O)OCc1ccccc1]

Glycolic acid (3.3 g, 43.39 mmol) and H-Ala-OBn·HCl (8.5 g 39.44 mmol) were dissolved in dichloromethane (100 mL), to which EDCI (11.3 g, 59.17 mmol) and diisopropylethylamine (13.0 ml, 78.89 mmol) were added. The reaction was stirred at r.t. until complete conversion, and then washed with water (50 ml), brine (50 ml), dried over anhydrous sodium sulfate, filtered and concentrated. The residue was purified by silica gel column (PE/EA=10/0 to 1/1 to 0/10) to give the title compound (2.9 g, 31% yield). MS-ESI (m/z): [M+H]⁺ calcd for C₁₂H₁₆NO₄, 238.10; found 238.10.

### Example 147. Synthesis of benzyl (2-((2-((((9H-fluoren-9-yl)methoxy)carbonyl) amino)acetamido)methoxy)acetyl)-L-alaninate (148)

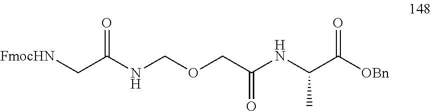

A mixture of compound 147 (2.9 g, 12.22 mmol) and compound 146 (4.5 g, 12.22 mmol) in toluene (60 ml), and catalytic amount of PPTS (0.3 g) was heated at 100° C. for 2 hours. The reaction was cooled to r.t. and the resulting white solid was filtered off, the filtrate was concentrated and diluted with 100 mL of ethyl acetate, washed with water (50 ml), dried over anhydrous sodium sulfate, filtered and concentrated, purified on silica gel column (PE/EA=10/0 to 1/1 to 1/3 to 0/10) to give the title compound (2.8 g, 42% yield). MS-ESI (m/z): [M+H]⁺ calcd for C₃₀H₃₂N₃O₇, 546.22; found 546.22.

### Example 148. Synthesis of (2-((2-((((9H-fluoren-9-yl)methoxy)carbonyl) amino)acetamido)methoxy)acetyl)-L-alanine (149)

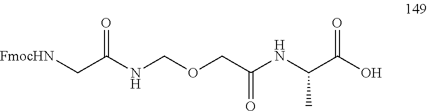

Compound 148 (2.0 g, 3.66 mmol) was dissolved in methanol (40 mL), 10% palladium on carbon (0.2 g) was added, and the reaction flask was evacuated and back-filled with hydrogen for three times. After stirring for 2 hours, the reaction mixture was filtered and the filtrate was concentrated to give the title compound 149 (1.45 g, 86% yield). MS-ESI (m/z): [M+H]⁺ calcd for C₂₃H₂₆N₃O₇, 456.17; found 456.17.

### Example 149. Synthesis of (9H-fluoren-9-yl)methyl (2-(((2-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-2-oxoethoxy)methyl)amino)-2-oxoethyl)carbamate (150)

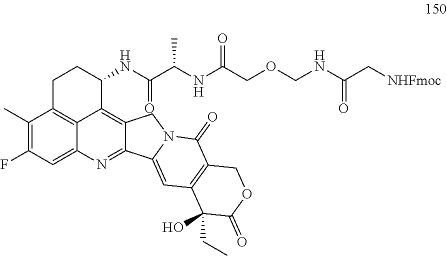

Compound 149 (300 mg, 0.659 mmol) and exatecan mesylate (350 mg, 0.659 mmol) were dissolved in DMF (10 mL), HATU (375 mg, 0.988 mmol) and diisopropylethylamine (217 μL, 1.317 mmol) were added. The reaction was stirred at r.t. until complete conversion, and then concentrated and purified by preparative HPLC to give the title compound 150 (400 mg, 70% yield). MS-ESI (m/z): [M+H]⁺ calcd for C₄₇H₄₅FN₆O₁₀, 873.32; found 873.32.

### Example 150. Synthesis of(S)-2-(2-((2-aminoacetamido)methoxy)acetamido)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propanamide (151)

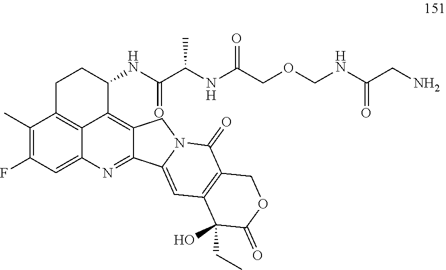 [SMILES: CC[C@@]1(O)C(=O)OCc2c1cc1n(c2=O)Cc2c-1nc1cc(F)c(C)c3c1c2[C@@H](NC(=O)[C@H](C)NC(=O)COCNC(=O)CN)CC3]

Compound 150 (170 mg, 0.195 mmol) was dissolved in a mixture of DMF (4 mL) and piperidine (0.4 mL), and the reaction was stirred at r.t. until completion. The solvent was removed, and the residue was co-evaporated with DMF (3 mL) to give the title compound, which was directly used in the next step without further purification (0.13 g, 100% yield). MS-ESI (m/z): [M+H]⁺ calcd for C₃₂H₃₅FN₆O₈, 651.25; found 651.26.

### Example 151. Synthesis of (2S,3S)-2,3-bis(2,5-dioxo-2,5-dihydro-1H-pyrrol-1-yl)-N1,N4-bis((S)-37-(((2S,13S)-1-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-2,14-dimethyl-1,4,9,12-tetraoxo-6-oxa-3,8,11-triazapentadecan-13-yl)carbamoyl)-31,39-dioxo-2,5,8,11,14,17,20,23,26,29-decaoxa-32,38-diazadotetracontan-42-yl)succinamide (152)

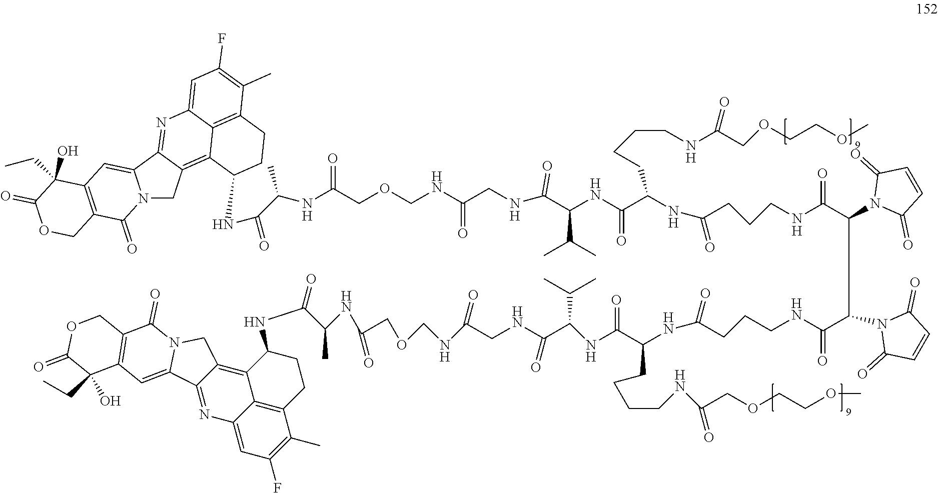

Compound 144 (180 mg, 0.096 mmol) and compound 151 (125 mg, 0.193 mmol) were dissolved in DMF (5 mL) and cooled to about 0° C. HATU (109 mg, 0.289 mmol) and diisopropylethylamine (31 L, 0.193 mmol) were added, and the reaction was warmed to r.t. and stirred until completion. The solvent was removed and the residue was purified by preparative HPLC to give compound 152 (101 mg, 34% yield). MS-ESI (m/z): [M+3H]³⁺ calcd for C₁₄₈H₂₁₀F₂N₂₂O₅₀, 1045.81; found 1046.10.

### Example 152. Synthesis of (2-((2-((((9H-fluoren-9-yl)methoxy)carbonyl)amino) acetamido)methoxy)acetyl)glycine (154)

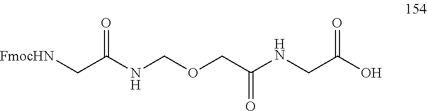

Compound 153 (600.3 mg, 1.1 mmol) was dissolved in 20 mL of MeOH, 10 wt % Pd/C (10.1 mg, 0.1 mmol) was added, and the reaction flask was evacuated and back-filled with hydrogen for three times. After stirring for 1 hour, the reaction mixture was filtered and the filtrate was concentrated, and triturated with ethyl acetate. The white solid was collected by filtration (420.2 mg, yield 84%). MS-ESI (m/z): [M+H]⁺ calcd for C₂₂H₂₄N₃O₇, 442.15; found: 442.15.

### Example 153. Synthesis of (9H-fluoren-9-yl)methyl (2-(((2-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)amino)-2-oxoethoxy)methyl)amino)-2-oxoethyl)carbamate (155)

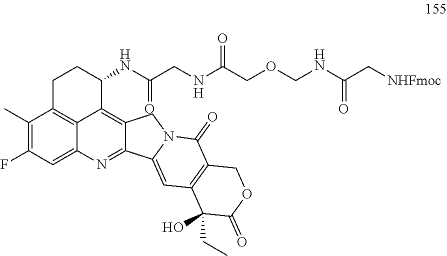

Compound 154 (302.1 mg, 0.68 mmol) was dissolved in 10 mL of DMF, and cooled over an ice-water bath to 0° C. Exatecan (325.1 mg, 0.61 mmol) and HATU (387.5 mmol, 1.01 mmol) were added, followed by diisopropylethylamine (180.2 μL, 1.36 mmol) dropwise. The reaction was stirred for about 20 min at r.t. and filtered. The filtrate was purified by preparative HPLC to give the title compound (135.2 mg, 23% yield). MS-ESI (m/z): [M+H]⁺ calcd for C₄₆H₄₄FN₆O₁₀, 859.30; found: 859.30.

### Example 154. Synthesis of 2-amino-N-((2-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)amino)-2-oxoethoxy)methyl)acetamide (156)

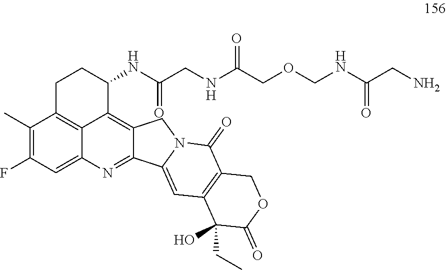 [SMILES: CC[C@@]1(O)C(=O)OCc2c1cc1n(c2=O)Cc2c-1nc1cc(F)c(C)c3c1c2[C@@H](NC(=O)CNC(=O)COCNC(=O)CN)CC3]

Compound 155 (135 mg, 0.157 mmol) was dissolved in 5 mL of DMF, 0.5 mL of piperidine was added, and the reaction was stirred at r.t. for 20 min, concentrated, re-dissolved in DMF, and concentrated again to give the title compound (100.9 mg, >100% yield). MS-ESI (m/z): [M+H]⁺ calcd for C₃₁H₃₄FN₆O₈, 637.23; found: 637.23.

### Example 155. Synthesis of (2S,3S)-2,3-bis(2,5-dioxo-2,5-dihydro-1H-pyrrol-1-yl)-N1,N4-bis((S)-37-(((S)-1-(((1S,9S)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-14-methyl-1,4,9,12-tetraoxo-6-oxa-3,8,11-triazapentadecan-13-yl)carbamoyl)-31,39-dioxo-2,5,8,11,14,17,20,23,26,29-decaoxa-32,38-diazadotetracontan-42-yl)succinamide (157)

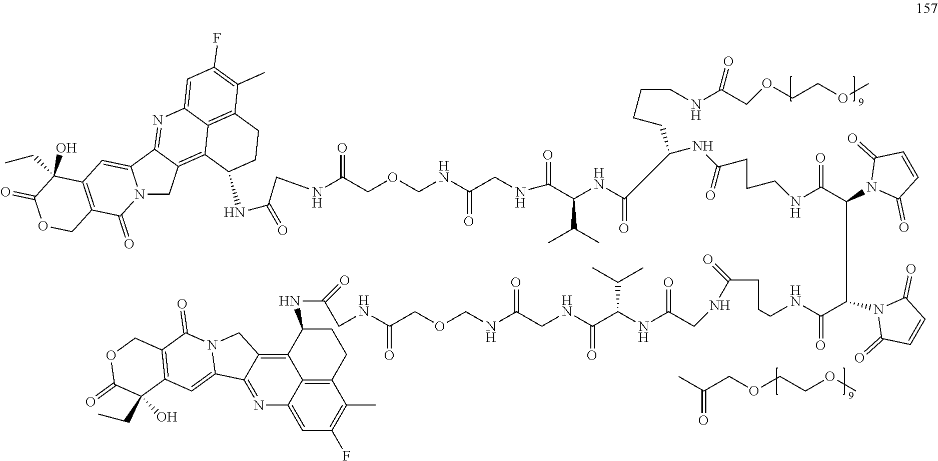

Compound 144 (140.2 mg, 0.075 mmol) and compound 156 (100.9 mg, 0.157 mmol) were dissolved in 5 mL of DMF, cooled to 0° C., and HATU (86.2 mg, 0.224 mmol) was added, followed by diisopropylethylamine (23.6 mg, 0.15 mmol) dropwise. The reaction was warmed to r.t. and after stirring for 20 min, the mixture was purified by preparative HPLC. The fractions were concentrated and lyophilized to give the title compound (36.6 mg, 16% yield). MS-ESI (m/z): [M+H]⁺ calcd for C₁₄₆H₂₀₇F₂N₂₂O₅₀, 3106.42; found: 3106.42.

### Example 156. Synthesis of 1-(2-amino-4-fluoro-5-methoxyphenyl)-2-chloroethan-1-one (158)

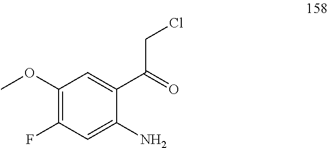

A solution of 3-fluoro-4-methoxyaniline (5 g, 35.4 mmol) in dichloromethane (20 mL) was added dropwise to an ice-water cooled boron trichloride (1 M in dichloromethane, 38.9 mL) solution. The reaction was stirred for 10 minutes and then chloroacetonitrile (3.2 g, 42.5 mmol) and aluminum trichloride (5.2 g, 38.9 mmol) were added. After the addition was completed, the reaction was warmed to r.t. and then refluxed overnight. The reaction mixture was then cooled to about 0° C., quenched with 2 M HCl (80 mL) and stirred at r.t. for 2 hours. Layers were separated and the aqueous phase was extracted with dichloromethane (3×80 mL). Combined organic phases were washed with water (100 mL), dried over sodium sulfate, filtered, concentrated, purified on a silica gel column, eluted with petroleum ether/ethyl acetate to give compound 158 (2 g, 26% yield) as a yellow solid. ESI-MS m/z: [M+H]⁺ calcd for C₉H₉ClFNO₂, 218.03; found 218.03.

### Example 157. Synthesis of (S)-11-(chloromethyl)-4-ethyl-8-fluoro-4-hydroxy-9-methoxy-1,12-dihydro-14H-pyrano[3′,4′:6,7]indolizino[1,2-b]quinoline-3,14(4H)-dione (160)

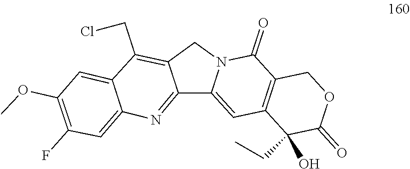 [SMILES: CC[C@@]1(O)C(=O)OCc2c1cc1n(c2=O)Cc2c-1nc1cc(F)c(OC)cc1c2CCl]

Compound 158 (0.50 g, 2.29 mmol) and compound 159 (0.57 g, 2.19 mmol) were dissolved in anhydrous toluene (40 mL), and p-toluenesulfonic acid (42 mg, 0.219 mmol) was added. The suspension was heated at reflux for 2 days and allowed to cool to r.t. After removal of about two-thirds of toluene, the residue was filtered and the filter cake was washed with dichloromethane, air-dried to give compound 160 (0.7 g, 72% yield) as a gray powdery solid. ESI-MS m/z: [M+H]⁺ calcd for C₂₂H₁₈ClFN₂O₅, 445.09; found 445.09.

### Example 158. Synthesis of tert-butyl (S)-(1-((4-(hydroxymethyl)phenyl)amino)-1-oxopropan-2-yl)carbamate (161)

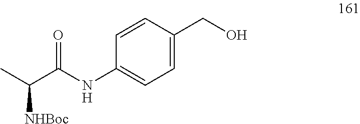 [SMILES: C[C@H](NC(=O)OC(C)(C)C)C(=O)Nc1ccc(CO)cc1]

p-Aminobenzyl alcohol (5.0 g, 0.04 mol) and Boc-L-alanine (8.0 g, 0.042 mol) were dissolved in anhydrous THF (100 mL), and 2-ethoxy-1-ethoxycarbonyl-1,2-dihydroquinoline (11 g, 0.044 mol) was added and stirred at r.t. overnight. The reaction mixture was poured into water (300 mL), extracted with ethyl acetate (3×100 mL), the combined organic phases were washed with water (100 mL), dried over sodium sulfate, filtered, and concentrated. The crude product was triturated with ethyl acetate/petroleum ether (1:3) and filtered to yield compound 161 (9.8 g, 84% yield) as a white solid. ESI-MS m/z: [M+H]⁺ calcd for C₁₅H₂₂N₂O₄, 295.16; found 295.16.

### Example 159. Synthesis of tert-butyl (S)-(1-((4-(bromomethyl)phenyl)amino)-1-oxopropan-2-yl)carbamate (162)

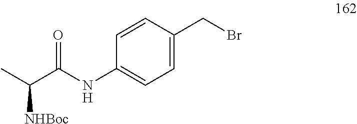 [SMILES: C[C@H](NC(=O)OC(C)(C)C)C(=O)Nc1ccc(CBr)cc1]

Compound 161 (3.5 g, 11.9 mmol) and carbon tetrabromide (5.9 g, 17.8 mmol) were dissolved in dichloromethane (80 mL), cooled to about 0° C., and triphenylphosphine (4.7 g, 17.8 mmol) was added. The reaction was warmed to r.t. and stirred for 30 minutes, and then 20 g of silica gel was added, mixed, and dried on a rotavap, loaded on a silica gel column (100 g of silica gel) and eluted with petroleum ether/ethyl acetate to yield compound 162 (2.6 g, 62% yield). ESI-MS m/z: [M+H]⁺ calcd for C₁₅H₂₁BrN₂O₃, 357.07; found 357.07.

### Example 160. Synthesis of (S)-4-(((9H-fluoren-9-yl)methoxy)carbonyl)-1-(4-(2-((tert-butoxycarbonyl)amino)propanamido)benzyl)-1-methylpiperazin-1-ium (164)

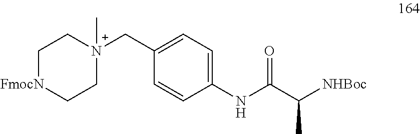

Compound 162 (2.3 g, 6.4 mmol) and (9H-fluoren-9-yl)methyl 4-methylpiperazine-1-carboxylate (163, 2.1 g, 6.4 mmol) were dissolved in anhydrous THF (100 mL) and stirred at r.t. overnight. After removal of most THF on a rotavap, ethyl acetate (200 mL) was added to the residue. The resulting slurry was filtered to give a white solid (3.8 g, 87% yield). ESI-MS m/z: M⁺ calcd for C₃₅H₄₃N₄O₅, 599.32; found 599.32.

### Example 161. Synthesis of (S)-1-(4-(2-((tert-butoxycarbonyl)amino) propanamido)benzyl)-1-methylpiperazin-1-ium (165)

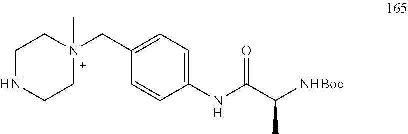 [SMILES: C[C@H](NC(=O)OC(C)(C)C)C(=O)Nc1ccc(C[N+]2(C)CCNCC2)cc1]

Compound 164 (3.12 g, 4.6 mmol) was dissolved in DMF (25 mL), and piperidine (3 mL) was added. After stirring at r.t. for 2 hours, 200 mL of ethyl acetate was added and stirred for 10 minutes. The mixture was filtered to give a white solid (1.54 g, 77% yield). ESI-MS m/z: M⁺ calcd for C₂₀H₃₃N₄O₃, 377.26; found 377.26.

### Example 162. Synthesis of 1-(4-((S)-2-((tert-butoxycarbonyl)amino) propanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium (166)

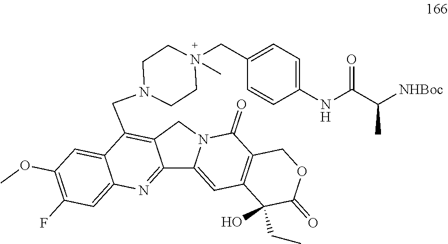

A mixture of compound 165 (0.30 g, 0.66 mmol), compound 160 (0.25 g, 0.56 mmol) in DMF (10 mL) was stirred at 0° C. for 30 minutes, then N, N-diisopropylethylamine (49 μL, 0.28 mmol) was added and the reaction was warmed to r.t. and stirred overnight, concentrated and purification by preparative HPLC (acetonitrile/water containing formic acid) to give compound 166 (0.40 g, 80% yield). ESI-MS m/z: M⁺ calcd for C₄₂H₅₀FN₆O₈, 785.37; found 785.37.

### Example 163. Synthesis of 1-(4-((S)-2-aminopropanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium (167)

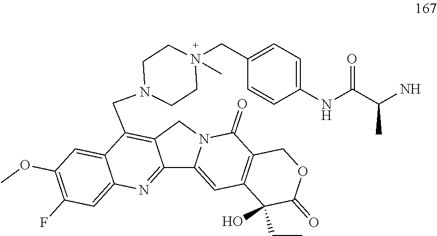

Compound 166 (0.30 g, 0.35 mmol) was dissolved in a mixture of dichloromethane and trifluoroacetic acid (3 mL/3 mL), and stirred at r.t. for 30 minutes. The mixture was then concentrated and dried on a vacuum pump to give compound 167 (0.27 g, 100% yield) as a yellow solid. ESI-MS m/z: M⁺ calcd for C₃₇H₄₂FN₆O₆, 685.31; found 685.31.

### Example 164. Synthesis of Compound 168

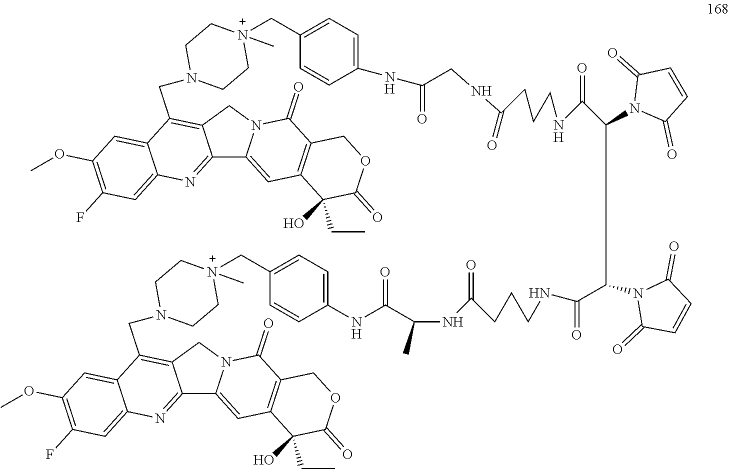

Compound 20 (50 mg, 0.1 mmol) and compound 167 (160 mg, 0.23 mmol) were dissolved in DMF (3 mL), HATU (120 mg, 0.3 mmol) and NMM (65 mg, 0.6 mmol) were added and stirred at r.t. for 2 hours. The reaction solution was directly purified by preparative HPLC (acetonitrile/water containing 0.1% HCOOH) to give 86 mg of compound 168 in 460% yield. ESI-MS m/z: M²⁺ calcd for C₉₄H₁₀₂F₂N₁₆O₂₀, 906.4; found 907.1.

### Example 165. Synthesis of 1-(2-amino-4-fluoro-5-methoxyphenyl)ethan-1-one (169)

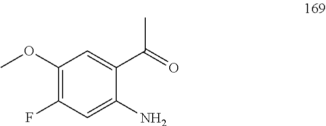

To a solution of 3-fluoro-4-methoxyaniline (5.0 g, 35.4 mmol) in dichloromethane (20 mL), was added BCl₃ (1 M, 39.0 mL) in dichloromethane at 0° C., followed by CH₃CN (2.2 mL, 42.5 mmol) and AlCl₃ (5.2 g, 38.9 mmol). The mixture was heated to reflux and stirred under reflux overnight, cooled to 0° C. 2N HCl (80 mL) was added and stirred for 2 hours, then extracted with ethyl acetate (3×50 mL). The organic phase was combined and washed with water (100 mL), dried over anhydrous Na₂SO₄, filtered and concentrated under vacuum. The residue was purified by a silica gel column to give a yellow solid (0.7 g, 11% yield). MS-ESI (m/z): [M+H]⁺ calcd for C₉H₁₁FNO₂, 184.07; found 184.07.

### Example 166. Synthesis of 1-(2-amino-4-fluoro-5-hydroxyphenyl)ethan-1-one (170)

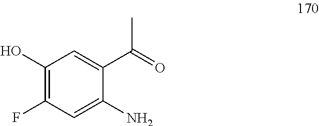

To a solution of compound 169 (0.71 g, 3.87 mmol) in dichloromethane (10 mL) was added BBr₃ (1 M, 7.7 mL). The solution was warmed to r.t. and stirred for 48 hours, cooled to 0° C. and quenched with water (100 mL), extracted with ethyl acetate (3×50 mL). The organic phases were combined and washed with water (100 mL), dried over anhydrous Na₂SO₄, filtered and concentrated under vacuum. The residue was purified by a silica gel column to give a yellow solid (0.4 g, 60% yield). MS-ESI (m/z): [M+H]⁺ calcd for C₈H₉FNO₂, 170.05; found 170.05.

### Example 167. Synthesis of(S)-4-ethyl-8-fluoro-4,9-dihydroxy-11-methyl-1,12-dihydro-14H-pyrano[3′,4′:6,7]indolizino[1,2-b]quinoline-3,14(4H)-dione (171)

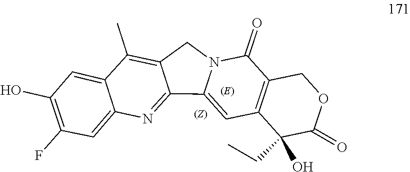 [SMILES: CC[C@@]1(O)C(=O)OCc2c1cc1n(c2=O)Cc2c-1nc1cc(F)c(O)cc1c2C]

To a solution of compound 170 (2.65 g, 10.05 mmol) and (S)-4-ethyl-4-hydroxy-7,8-dihydro-1H-pyrano[3,4-f]indolizine-3,6,10(4H)-trione (2.65 g, 10.05 mmol) in toluene (80 mL) was added p-toluenesulfonic acid (0.1 g), and the mixture was heated to reflux, stirred for 24 hours, and cooled to r.t. The resulting solid was filtered, rinsed with dichloromethane and dried to give compound 171 (1.3 g, 100% yield). MS-ESI (m/z): [M+H]⁺ calcd for C₂₁H₁₈FN₂O₅, 397.11; found 397.11.

### Example 168. Synthesis of (S)-1-(tert-butyl) 4-(4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl) piperazine-1,4-dicarboxylate (172)

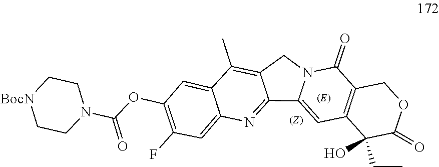 [SMILES: CC[C@@]1(O)C(=O)OCc2c1cc1n(c2=O)Cc2c-1nc1cc(F)c(OC(=O)N3CCN(C(=O)OC(C)(C)C)CC3)cc1c2C]

To a solution of tert-butyl 1-piperazinecarboxylate (1.0 g, 5.36 mmol) and pyridinium (0.65 ml, 8.05 mmol) in dichloromethane (5 mL) was added triphosgene (1.9 g, 6.44 mmol) at 0° C. The mixture was warmed to r.t. and stirred for 1 hours, and then diluted with dichloromethane (50 mL) and washed with 1N HCl (10 mL), dried over anhydrous Na₂SO₄, filtered and concentrated under vacuum to give a yellow solid (1.3 g, 100% yield).

To a solution of compound 171 (400 mg, 1.01 mmol) and diisopropylethylamine (0.33 mL, 2.02 mol) in DMF (5 mL) was added the above compound (326 mg, 1.31 mmol) at 0° C. The mixture was warmed to r.t. and stirred for 4 hours, then concentrated under vacuum and purified by a silica gel column to give a brown solid (430 mg, 70% yield). MS-ESI (m/z): [M+H]⁺ calcd for C₃₁H₃₃FN₄O₈, 609.23; found 609.23.

### Example 169. Synthesis of (S)-4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl piperazine-1-carboxylate (173)

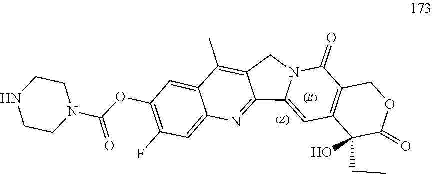 [SMILES: CC[C@@]1(O)C(=O)OCc2c1cc1n(c2=O)Cc2c-1nc1cc(F)c(OC(=O)N3CCNCC3)cc1c2C]

Compound 172 (100 mg, 0.164 mmol) was dissolved in dichloromethane (6 mL) and trifluoroacetic acid (2 mL), and the reaction was stirred for 30 min, and then concentrated, co-evaporated with dichloromethane twice, dried on an oil pump to give compound 173 as a brown solid (83 mg, 100.00%). MS-ESI (m/z): [M+H]⁺ calcd for C₂₆H₂₆FN₄O₆, 509.18; found 509.18.

### Example 170. Synthesis of tert-butyl ((S)-37-(((benzyloxy)carbonyl)amino)-31-oxo-2,5,8,11,14,17,20,23,26,29-decaoxa-32-azaoctatriacontan-38-oyl)glycylglycyl-L-alanyl-L-alaninate (174)

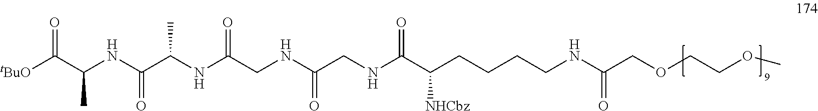

To a solution of compound 46 (3.02 g, 3.5 mmol) and H-Ala-Ala-O^{t}Bu (0.9 g, 3.56 mmol) in THF (60 mL), were added HATU (1.99 g, 5.23 mmol) and diisopropylethylamine (1.1 mL, 6.95 mmol). The reaction was stirred at r.t. until complete conversion, and then concentrated under vacuum and poured into water (100 mL), extracted with dichloromethane (3×50 mL). The combined organic phases were washed with water (50 mL), saturated sodium bicarbonate (50 mL), 2 N HCl (50 mL), and brine (50 mL), dried over sodium sulfate, filtered and concentrated to give compound 174 (3.6 g, 100% yield). MS-ESI (m/z): [M+H]⁺ calcd for C₄₉H₈₄N₆O₁₉, 1061.58; found 1061.58.

### Example 171. Synthesis of tert-butyl ((S)-37-amino-31-oxo-2,5,8,11,14,17,20,23, 26,29-decaoxa-32-azaoctatriacontan-38-oyl)glycylglycyl-L-alanyl-L-alaninate (175)

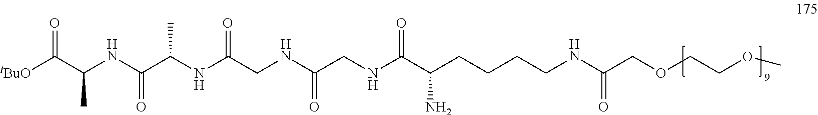

Compound 174 (3.6 g, 3.88 mmol) was dissolved in THF (60 mL), palladium on carbon (10 wt %, 0.4 g) was added, and the reaction flask was evacuated and back-filled with hydrogen for three times. After stirring for 2 hours, the reaction mixture was filtered and the filtrate was concentrated to give the title compound 175 (2.6 g, 74% yield). MS-ESI (m/z): [M+H]⁺ calcd for C₄₁H₇₉N₆O₁₇, 927.54; found 927.54.

### Example 172. Synthesis of di-tert-butyl (2S,5S,14S,22S,23S,31S,40S,43S)-22,23-bis(2,5-dioxo-2,5-dihydro-1H-pyrrol-1-yl)-2,5,40,43-tetramethyl-4,7,10,13,16,21,24,29, 32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioate (176)

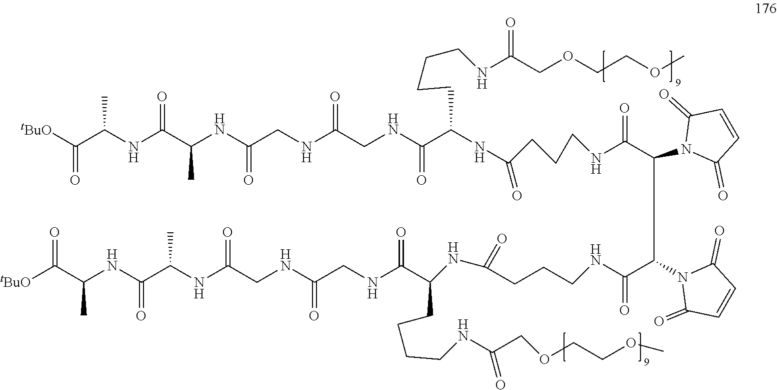

To a solution of compound 175 (2.6 g, 2.8 mmol) and compound 19 (0.6 g, 1.25 mmol) in THF/DMF (20 mL/4 mL), were added HATU (1.43 g, 3.76 mmol) and diisopropylethylamine (0.62 mL, 3.79 mmol). The reaction was stirred at r.t. until complete conversion, and then concentrated under vacuum and poured into water (200 mL), extracted with dichloromethane (3×100 mL). The combined organic phases were washed with water (50 mL) and brine (50 mL), dried over sodium sulfate, filtered and concentrated to give compound 176 (2.8 g, 100% yield). MS-ESI (m/z): [M+H]⁺ calcd for C₁₀₂H₁₇₅N₁₆O₄₂, 2296.20; found 2296.20.

### Example 173. Synthesis of (2S,5S,14S,22S,23S,31S,40S,43S)-22,23-bis(2,5-dioxo-2,5-dihydro-1H-pyrrol-1-yl)-2,5,40,43-tetramethyl-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioic acid (177)

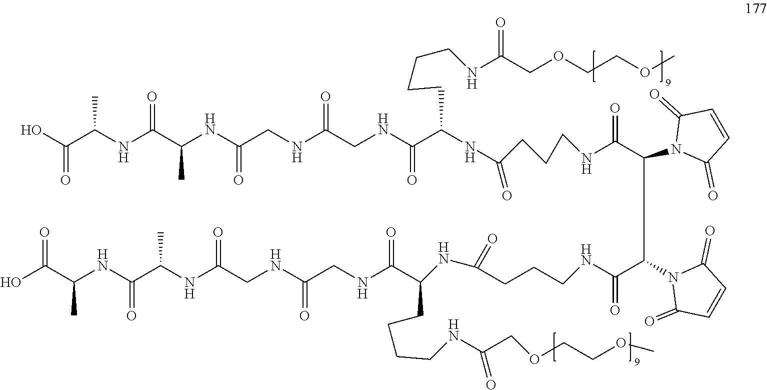

Compound 176 (2.8 g, 1.2 mmol) was dissolved in formic acid (40 mL) and dichloromethane (20 mL). The mixture was heated to 50° C. and stirred overnight, and then concentrated under vacuum, purified by preparative HPLC to give compound 177 (1.6 g, 61% yield). MS-ESI (m/z): [M+H]⁺ calcd for C₉₄H₁₅₉N₁₆O₄₂, 2184.07; found 2184.07.

### Example 174. Synthesis of bis((S)-4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl) 4,4′-((2S,5S,14S,22S,23S,31S,40S,43S)-22,23-bis(2,5-dioxo-2,5-dihydro-1H-pyrrol-1-yl)-2,5,40,43-tetramethyl-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30, 33,36,39,42-dodecaazatetratetracontanedioyl)bis(piperazine-1-carboxylate) (178)

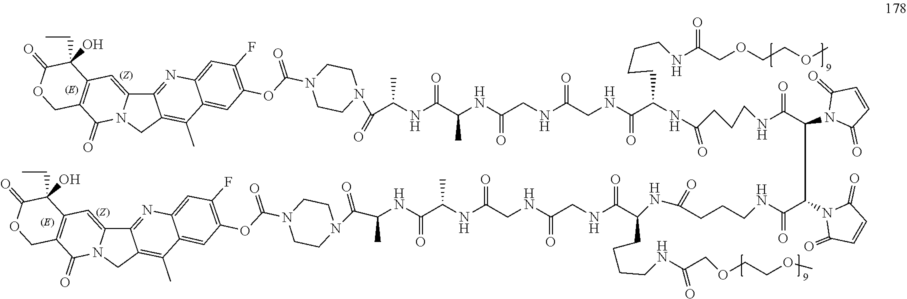

To a solution of compound 177 (125 mg, 0.057 mmol) and compound 173 (58 mg, 0.115 mmol) in DMF (5 mL), were added HATU (65 mg, 0.173 mmol) and diisopropylethylamine (38 μL, 0.230 mmol). The reaction was stirred at r.t. until complete conversion, and then concentrated and purified by preparative HPLC to give compound 178 (67 mg, 37% yield). MS-ESI (m/z): [M+H]⁺ calcd for C₁₄₆H₂₀₅F₂N₂₄O₅₂, 3164.40; found 3164.40.

### Example 175. Synthesis of (S)-tert-butyl (4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl) ethane-1,2-diylbis(methylcarbamate) (179)

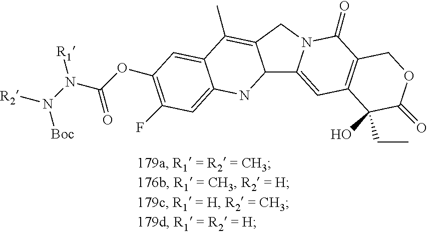

To a solution of tert-butyl 2-(methylamino)ethylcarbamate (1.0 g, 5.74 mmol) and pyridinium (0.69 ml, 8.61 mmol) in dichloromethane (20 mL) was added triphosgene (2.0 g, 6.89 mmol) at 0° C. The mixture was warmed to r.t. and stirred for 1 hour, diluted with dichloromethane (50 mL) and washed with 1N HCl (10 mL), dried over anhydrous Na₂SO₄, filtered and concentrated under vacuum to give a yellow oil (1.3 g, 100% yield).

To a solution of compound 171 (712 mg, 1.79 mmol) and diisopropylethylamine (0.59 ml, 3.59 mol) in DMF (10 mL) were added the above compound (710 mg, 3.00 mmol) and DMAP (43 mg, 0.36 mmol) at 0° C. The reaction was warmed to r.t. and stirred overnight, then concentrated under vacuum and purified by a silica gel column to give a brown solid (700 mg, 65% yield). MS-ESI (m/z): [M+H]⁺ calcd for C₃₀H₃₄FN₄O₈, 597.23; found 597.23.

### Example 176. Synthesis of (S)-4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl methyl(2-(methylamino)ethyl)carbamate (180)

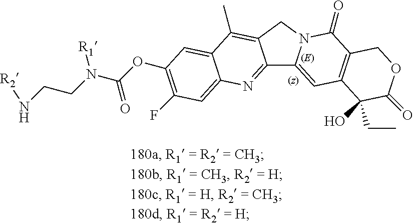

Compound 179 (55 mg, 0.092 mmol) was dissolved in dichloromethane (3 mL). trifluoroacetic acid (1 mL) was added and the reaction was stirred for 30 min, and then concentrated, co-evaporated with dichloromethane twice, dried on an oil pump to give compound 180 as a brown solid (50 mg, >100.00% yield). MS-ESI (m/z): [M+H]⁺ calcd for C₂₅H₂₆FN₄O₆, 497.18; found 497.18.

### Example 177. Synthesis of bis((S)-4-ethyl-8-fluoro-4-hydroxy-11-methyl-3,14-dioxo-3,4,12,14-tetrahydro-1H-pyrano[3′,4′:6,7]indolizino[1,2-b]quinolin-9-yl) ((5S,8S,17S,25S,26S,34S,43S,46S)-25,26-bis(2,5-dioxo-2,5-dihydro-1H-pyrrol-1-yl)-3,5,8,43,46,49-hexamethyl-4,7,10,13,16,19,24,27,32,35,38,41,44,47,48-pentadecaoxo-17,34-bis(31-oxo-2,5,8,11,14,17,20,23,26,29-decaoxa-32-azahexatriacontan-36-yl)-3,6,9,12,15,18,23,28,33,36,39,42,45,49-tetradecaazahenpentacontane-1,51-diyl)bis(methylcarbamate) (181)

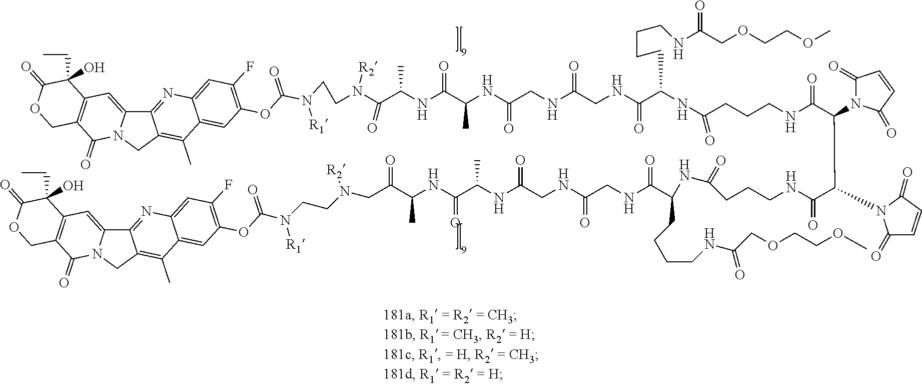

To a solution of compound 173 (168 mg, 0.08 mmol) and compound 180 (81 mg, 0.16 mmol) in DMF (5 mL), were added HATU (100 mg, 0.26 mmol) and diisopropylethylamine (52 μL, 0.32 mmol). The reaction was stirred at r.t. until complete conversion, and then concentrated and purified by preparative HPLC to give compound 181 (97 mg, 39% yield). MS-ESI (m/z): [M+H]⁺ calcd for C₁₄₄H₂₀₇F₂N₂₂O₅₀, 3081.42, found 3081.42.

### Example 178. Synthesis of (4-(4-((4-(((S)-1-carboxy-2-methylpropyl)amino)-4-oxobutyl)amino)-2,3-bis(2,5-dioxo-2,5-dihydro-1H-pyrrol-1-yl)-4-oxobutanamido) butanoyl)-L-valine (184)

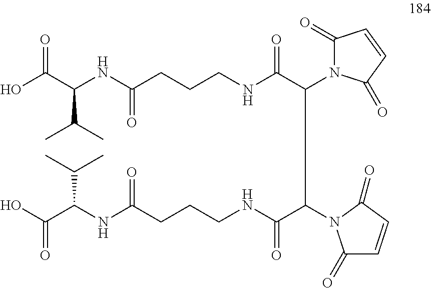

To a mixture of compound 12 (2.0 g, 4.18 mmol) in anhydrous acetonitrile (50 mL) were added 3-(ethyliminomethylideneamino)-N,N-dimethylpropan-1-amine hydrochloride (3.2 g, 16.72 mmol) and N-hydroxysuccinimide (1.9 g, 16.72 mmol). The mixture was stirred at r.t. until complete conversion and diluted with DCM (100 mL) and washed with brine (200 mL). The organic layer was dried over Na₂SO₄ and concentrated under vacuum to give an off-white solid.

The obtained compound was dissolved in DMF (50 mL) and valine (1.5 g, 12.54 mmol) was added. The mixture was stirred at r.t. until complete conversion and then directly purified by preparative HPLC to give compound 184 (2.2 g, 78% yield) as an off-white solid. MS-ESI (m/z): [M+H]⁺ calcd for C₃₀H₄₀N₆O₁₂, 677.27; found, 677.53.

### Example 179. Synthesis of 2,5-dioxopyrrolidin-1-yl (4-(2,3-bis(2,5-dioxo-2,5-dihydro-1H-pyrrol-1-yl)-4-((4-(((R)-1-((2,5-dioxopyrrolidin-1-yl)oxy)-3-methyl-1-oxobutan-2-yl)amino)-4-oxobutyl)amino)-4-oxobutanamido)butanoyl)-L-valinate (185)

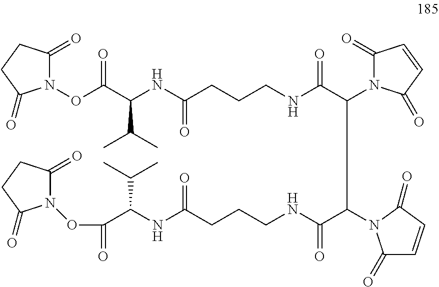

To a mixture of compound 184 (1.0 g, 1.48 mmol) in anhydrous acetonitrile (40 mL) were added 3-(ethyliminomethylideneamino)-N,N-dimethylpropan-1-amine hydrochloride (1.2 g, 5.92 mmol) and N-hydroxysuccinimide (685 mg, 5.92 mmol). The mixture was stirred at r.t. until complete conversion, diluted with DCM (50 mL) and washed with brine (150 mL). The organic layer was dried over Na₂SO₄ and concentrated under vacuum to give compound 185 (1.2 g, 93% yield) as an off-white solid. MS-ESI (m/z): [M+H]⁺ calcd for C₃₈H₄₆N₈O₁₆, 871.30; found, 871.65.

### Example 180. Synthesis of (S)-1-(4-(2-((tert-butoxycarbonyl)amino)propanamido) benzyl)-4-(chlorocarbonyl)-1-methylpiperazin-1-ium (187)

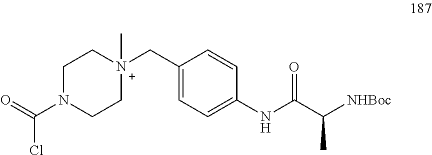 [SMILES: C[C@H](NC(=O)OC(C)(C)C)C(=O)Nc1ccc(C[N+]2(C)CCN(C(=O)Cl)CC2)cc1]

To a solution of compound 186 (300 mg, 0.796 mmol) in DCM (15 mL), was added triphosgene (98 mg, 0.318 mmol) at 0° C. The reaction was stirred at 0° C. until complete conversion, and then concentrated and purified by preparative HPLC to give compound 187 (190 mg, 54% yield). MS-ESI (m/z): [M]⁺ calcd for C₂₁H₃₂ClN₄O₄, 439.20; found 439.35.

### Example 181. Synthesis of Compound 188

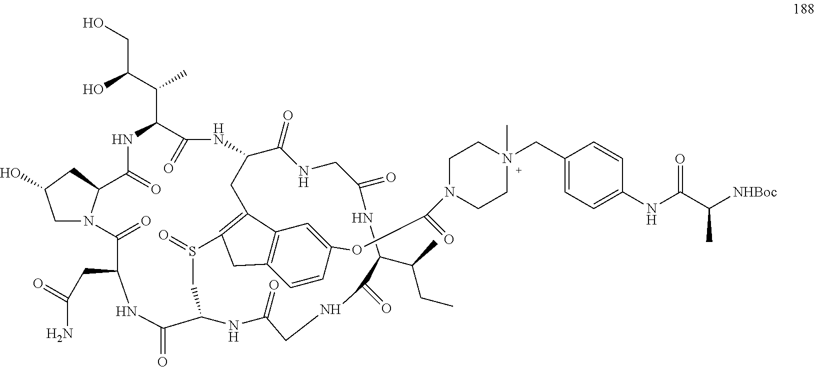

To a solution of α-amanitin (30 mg, 0.033 mmol) and compound 187 (42.9 mg, 0.099 mmol) in DMF (1 mL) was added diisopropylethylamine (12.6 mg, 0.099 mmol) at 0° C. The reaction was stirred at 0° C. until complete conversion and then purified by preparative HPLC to give compound 189 (36 mg, 83% yield). MS-ESI (m/z): [M]⁺ calcd for C₆₀H₈₅N₁₄O₁₈S, 1321.58; found 1321.69.

### Example 182. Synthesis of Compound 189

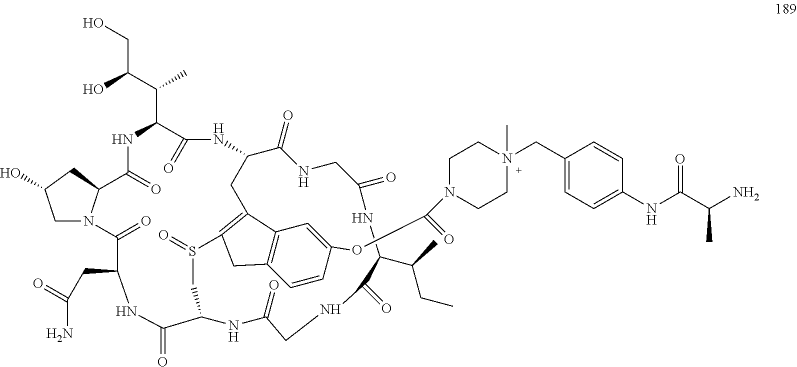

A solution of compound 188 (36 mg, 27.2 umol) in TFA/DCM (1/10, 1 mL) was stirred at r.t. until complete conversion. The reaction mixture was concentrated under vacuum to give compound 189 (35 mg, crude product, 100% yield). MS-ESI (m/z): [M]⁺ calcd for C₅₅H₇₇N₁₄O₁₆S⁺, 1221.58; found 1221.70.

### Example 183. Synthesis of Compound 190

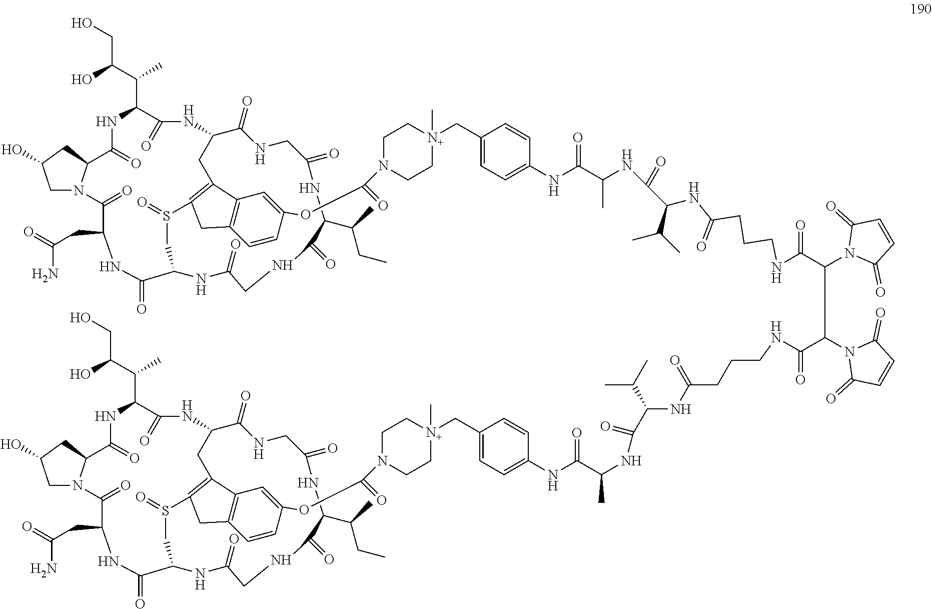

To a mixture of compound 185 (4 mg, 459 μmol) and 189 (11.8 mg, 9.65 μmol) in anhydrous DMF (2 mL) was added diisopropylethylamine (1.5 mg, 11.47 μmol) at 0° C. The mixture was stirred at 0° C. until complete conversion, and directly purified by preparative HPLC to give compound 190 (8.6 mg, 600% yield) as an off-white solid. MS-ESI (m/z): [M]⁺ calcd for C₁₄₀H₁₉N₃₄O₄₂S₂, 1541.66; found, 1541.69.

### Example 184. Synthesis of bis(2,5-dioxopyrrolidin-1-yl) (2S,5S,13S,14S,22S,25S)-13,14-bis(2,5-dioxo-2,5-dihydro-1H-pyrrol-1-yl)-2,25-diisopropyl-4,7,12,15,20,23-hexaoxo-5,22-bis(31-oxo-2,5,8,11,14,17,20,23,26,29-decaoxa-32-azahexatriacontan-36-yl)-3,6,11,16,21,24-hexaazahexacosanedioate (191)

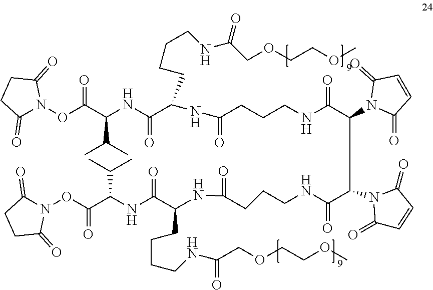

To a mixture of compound 144 (100 mg, 53.47 umol) in anhydrous DCM (5 mL) were added 3-(ethyliminomethylideneamino)-N,N-dimethylpropan-1-amine hydrochloride (41 mg, 214 umol) and N-hydroxysuccinimide (25 mg, 214 umol). The mixture was stirred at 0° C. until complete conversion, and then diluted with DCM (10 mL), washed with brine (2×10 mL). The organic layer was dried over Na₂SO₄ and concentrated under vacuum to give compound 191 (100 mg, 91% yield) as an oil. MS-ESI (m/z): [M+H]⁺ calcd for C₉₂H₁₅₀N₁₂O₄₀, 2064.01; found, 2064.12.

### Example 185. Synthesis of Compound 192

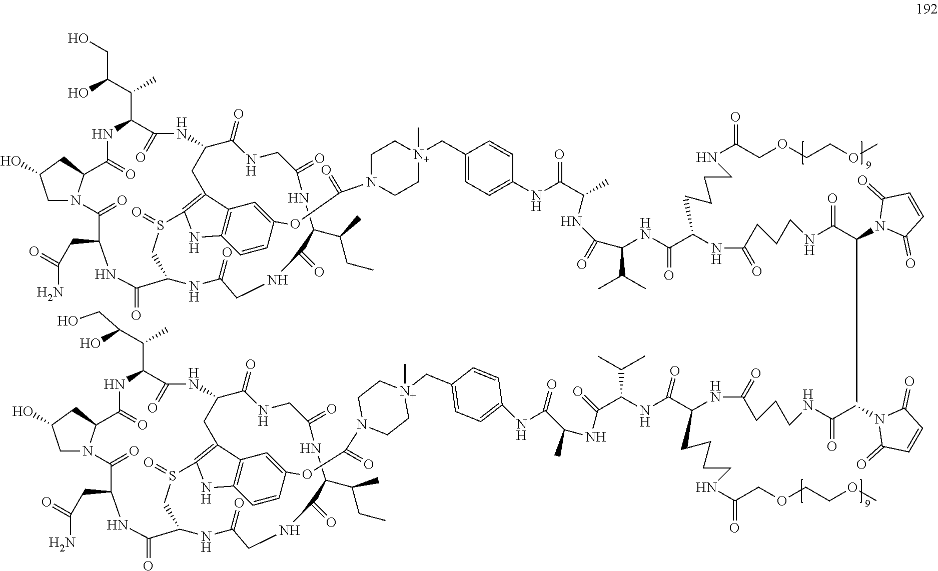

To a mixture of compound 191 (8 mg, 3.88 μmol) and 189 (10 mg, 8.14 μmol) in anhydrous DMF (2 mL) were added diisopropylethylamine (1.5 mg, 11.47 umol) at 0° C. The mixture was stirred at 0° C. until complete conversion and directly purified by preparative HPLC to give compound 192 (10.6 mg, 64% yield) as an off-white solid. MS-ESI (m/z): [M]⁺ calcd for C₁₉₄H₂₉₄N₃₈O₆₆S₂, 2138.03; found, 2138.03.

### Example 186. Synthesis of (2S,5S,22S,25S)-13,14-bis(2,5-dioxo-2,5-dihydro-1H-pyrrol-1-yl)-5,22-diisopropyl-2,25-dimethyl-4,7,12,15,20,23-hexaoxo-3,6,11,16,21,24-hexaazahexacosanedioic acid (193)

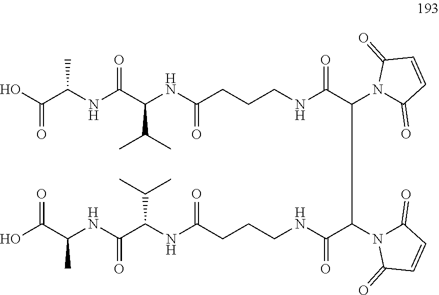 [SMILES: CC(C)[C@H](NC(=O)CCCNC(=O)C(C(C(=O)NCCCC(=O)N[C@H](C(=O)N[C@@H](C)C(=O)O)C(C)C)N1C(=O)C=CC1=O)N1C(=O)C=CC1=O)C(=O)N[C@@H](C)C(=O)O]

To a mixture of compound 185 (600 mg, 689 μmol) and L-analine (184 mg, 2.07 mmol) in anhydrous DMF (20 mL) were added diisopropylethylamine (267 mg, 2.07 mmol) at 0° C. The mixture was stirred at 0° C. until complete conversion and directly purified by preparative HPLC to give compound 193 (510 mg, 91% yield) as an off-white solid. MS-ESI (m/z): [M+H]⁺ calcd for C₃₆H₅₀N₈O₁₄, 819.36; found, 819.53.

### Example 187. Synthesis of 2,3-bis(2,5-dioxo-2,5-dihydro-1H-pyrrol-1-yl)-N1,N4-bis(4-(((S)-3-methyl-1-oxo-1-(((S)-1-oxo-1-((4-oxocyclohexyl)amino)propan-2-yl)amino)butan-2-yl)amino)-4-oxobutyl)succinamide (194)

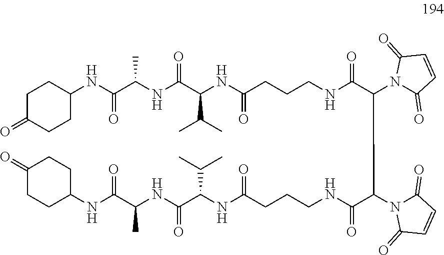 [SMILES: CC(C)[C@H](NC(=O)CCCNC(=O)C(C(C(=O)NCCCC(=O)N[C@H](C(=O)N[C@@H](C)C(=O)NC1CCC(=O)CC1)C(C)C)N1C(=O)C=CC1=O)N1C(=O)C=CC1=O)C(=O)N[C@@H](C)C(=O)NC1CCC(=O)CC1]

To a mixture of compound 193 (100 mg, 122 μmol) and 4-aminocyclohexan-1-one hydrochloride (55 mg, 366 umol) in anhydrous DMF (2 mL) were added 4-(4,6-dimethoxy-1,3,5-triazin-2-yl)-4-methyl morpholinium chloride (DMTMM, 135 mg, 488 μmol) at 0° C. The mixture was stirred at 0° C. until complete conversion, diluted with DCM (10 mL) and washed with brine (3×10 mL). The organic layer was dried over Na₂SO₄ and concentrated under vacuum. The residue was purified by flash column to give compound 194 (110 mg, 89% yield) as an off-white solid. MS-ESI (m/z): [M+H]⁺ calcd for C₄₈H₆₈N₁₀O₁₄, 1009.49; found, 1009.68.

### Example 188. Synthesis of Compound 195

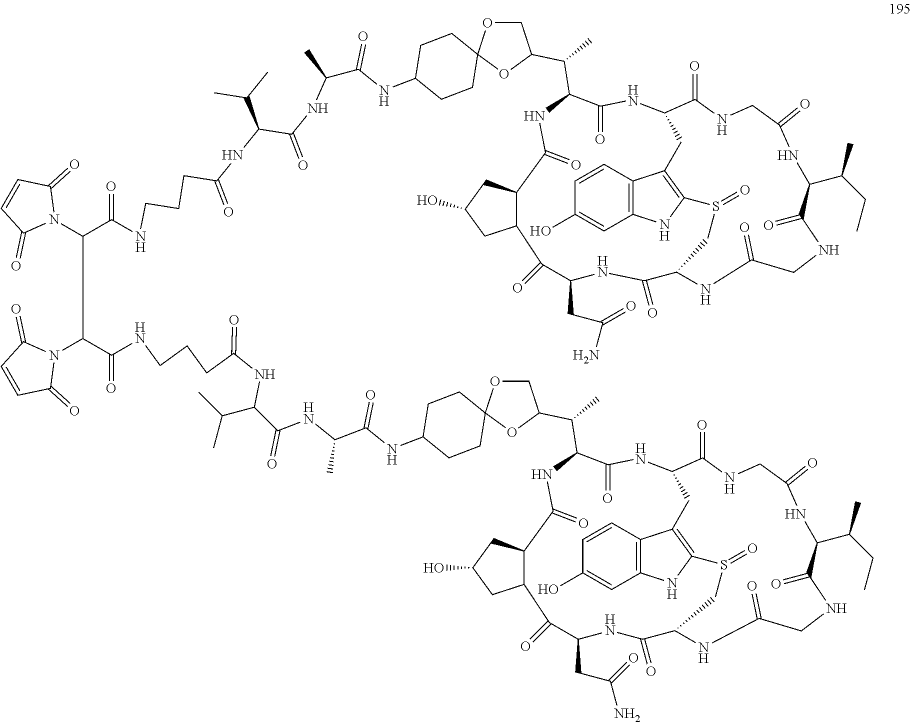

A mixture of compound 191 (5 mg, 4.95 μmol), toluene-4-sulfonic acid (1 mg, 5.86 μmol) and α-amantin (182, 9.6 mg, 10.41 μmol) in anhydrous THF (5 mL) was stirred at 60° C. until complete conversion. The mixture was concentrated under vacuum and purified by preparative HPLC to give compound 195 (4.6 mg, 33% yield) as an off-white solid. MS-ESI (m/z): [M+H]⁺ calcd for C₁₂₈H₁₇₄N₂₈O₄₂S₂, 2840.18; found, 2840.75.

### Example 189. Synthesis of 3-(4-((((9H-fluoren-9-yl)methoxy)carbonyl)amino) phenyl)acrylic acid (197)

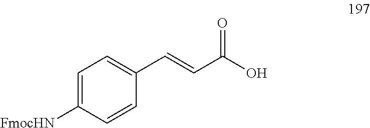

To a mixture of 4-aminocinnamic acid (1.0 g, 6.13 mmol) and N-(9-fluorenylmethoxycarbonyloxy) succinimide (2.3 g, 6.74 mmol) in DCM (20 mL) was added trimethylamine (930 mg, 9.21 mmol) at 0° C. The mixture was stirred at 0° C. until complete conversion, and then washed with brine (20 mL), dried over Na₂SO₄ and concentrated under vacuum. The residue was purified by flash column to give compound 197 (2.2 g, 93% yield) as a white solid. MS-ESI (m/z): [M+H]⁺ calcd for C₂₄H₁₉NO₄, 386.13; found, 386.24.

### Example 190. Synthesis of (9H-fluoren-9-yl)methyl (E)-(4-(3-(methoxy(methyl) amino)-3-oxoprop-1-en-1-yl)phenyl)carbamate (198)

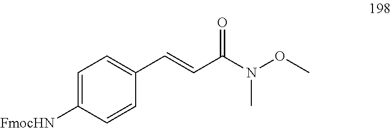

To a mixture of compound 197 (2.0 g, 5.19 mmol) and N-methoxymethanamine (610 mg, 6.23 mmol) in DCM (20 mL) were added HATU (3.0 g, 7.80 mmol) and trimethylamine (1.1 g, 10.20 mmol) at 0° C. The mixture was stirred at 0° C. until complete conversion, and then washed with brine (20 mL), dried over Na₂SO₄ and concentrated under vacuum. The residue was purified by flash column to give compound 198 (1.8 g, 81% yield) as a white solid. MS-ESI (m/z): [M+H]⁺ calcd for C₂₆H₂₄N₂O₄,429.17; found, 429.30

### Example 191. Synthesis of (9H-fluoren-9-yl)methyl (E)-(4-(3-oxoprop-1-en-1-yl)phenyl)carbamate (199)

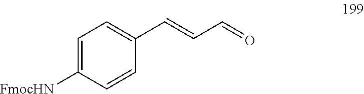

A mixture of compound 198 (1.8 g, 4.20 mmol) in DCM (20 mL) was cooled down to −60° C. under N₂. Diisobutylaluminium hydride (12.6 mL, 12.6 mmol, 1.0 M in THF) was added dropwise to the mixture. After additional, the mixture was stirred at −60° C. until complete conversion. The reaction was quenched with 10% NH₄Cl, and then washed brine (20 mL), dried over Na₂SO₄ and concentrated under vacuum. The residue was purified by flash column to give compound 199 (900 mg, 58% yield) as an off-white solid. MS-ESI (m/z): [M+H]⁺ calcd for C₂₄H₁₉NO₃, 370.17; found, 370.58.

### Example 192. Synthesis of Compound 200

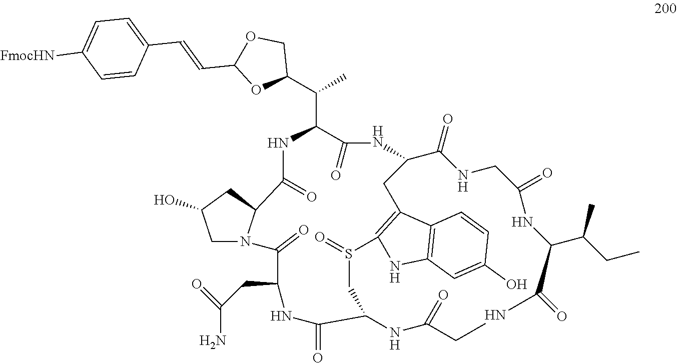

A mixture of compound 199 (8.8 mg, 23.94 μmol), toluene-4-sulfonic acid (1 mg, 5.86 μmol) and α-amantin (20.0 mg, 21.76 μmol) in anhydrous THF (5 mL) was stirred at 60° C. until complete conversion and concentrated under vacuum. The residue was purified by flash column to give compound 200 (16 mg, 57.9% yield). MS-ESI (m/z): [M+H]⁺ calcd for C₆₃H₇₁N₁₁O₁₆S, 1270.48; found, 1270.85.

### Example 193. Synthesis of Compound 201

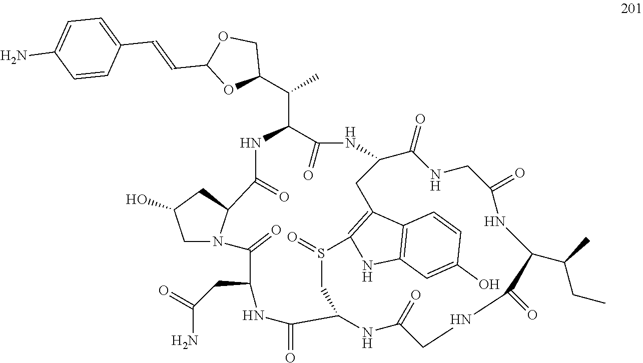

A solution of compound 200 (16 mg, 12.60 μmol) in piperidine/DMF (1 mL, 1/10) was stirred at r.t. for 10 min and concentrated under vacuum to give compound 201 (16 mg, crude). MS-ESI (m/z): [M+H]⁺ calcd for C₄₈H₆₁N₁₁O₁₄S, 1048.48; found, 1048.85.

### Example 194. Synthesis of Compound 202

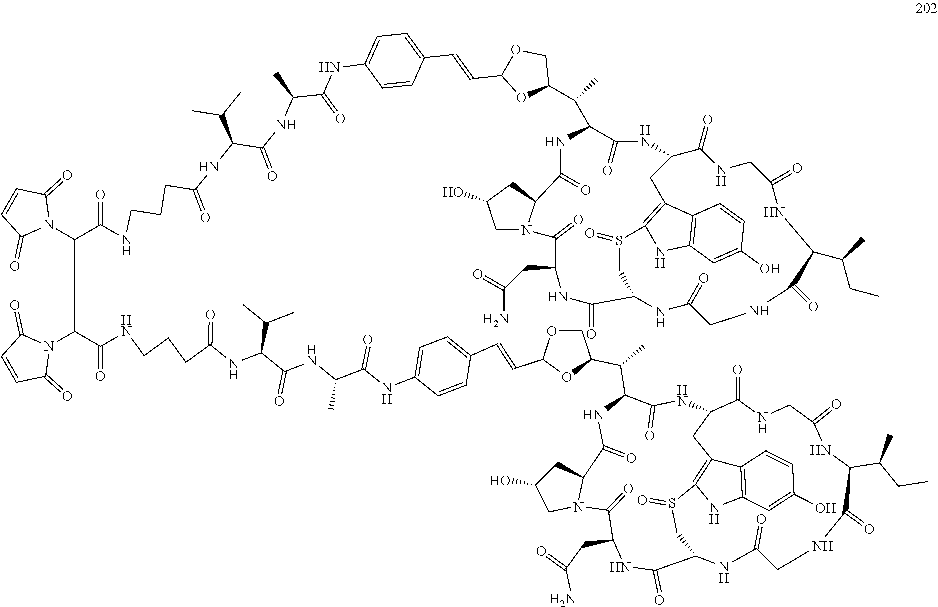

To a mixture of compound 193 (5 mg, 6.11 μmol) and compound 201 (13 mg, 12.81 μmol) in anhydrous DMF (1 mL) were added DMTMM (4-(4,6-dimethoxy-1,3,5-triazin-2-yl)-4-methyl morpholinium chloride) (7 mg, 24.44 μmol) at 0° C. The mixture was stirred at 0° C. until complete conversion and then purified by preparative HPLC to give compound 202 (10 mg, 57% yield) as an off-white solid. MS-ESI (m/z): [M+H]⁺ calcd for C₁₃₂H₁₆₈N₃₀O₄₀S₂, 2878.44; found, 2878.92.

### Example 195. Synthesis of (2R,3R)-2,3-bis(((benzyloxy)carbonyl)amino)-4-((4-(tert-butoxy)-4-oxobutyl)amino)-4-oxobutanoic acid (203)

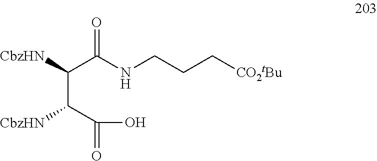 [SMILES: CC(C)(C)OC(=O)CCCNC(=O)[C@H](NC(=O)OCc1ccccc1)[C@@H](NC(=O)OCc1ccccc1)C(=O)O]

To a mixture of compound 13 (4.25 g, 10.68 mmol, 1.0 eq) and DMAP (13 mg, 0.11 mmol, 0.01 eq) in 20 mL of dry DCM was added a solution of t-butyl aminobutyrate (1.78 g, 11.21 mmol, 1.05 eq) in 10 mL of anhydrous DCM. After the addition was completed, compound 13 was completely dissolved and the reaction was allowed to stir at r.t. overnight. The crude product was loaded on a silica gel column and eluted with 3-5% MeOH/DCM. Fractions were combined and concentrated, the residue was triturated with PE/DCM (1:1) to afford 3.3 g of a white solid (yield 56%). MS-ESI (m/z): [M+H]⁺ calcd. for C₂₈H₃₆N₃O₉ 558.2; found, 558.2.

### Example 196. Synthesis of 1-benzyl 25-(tert-butyl) (18R,19R)-18,19-bis(((benzyloxy)-carbonyl)amino)-17,20-dioxo-4,7,10,13-tetraoxa-16,21-diazapentacosanedioate (204)

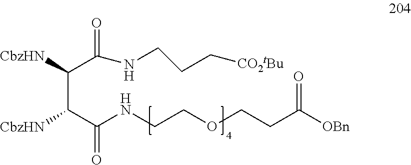

Benzyl 1-amino-3,6,9,12-tetraoxapentadecan-15-oate (365 mg, 1.03 mmol, 1.0 eq) was dissolved in 10 mL of DMF, cooled over ice water bath. To which DIPEA (0.53 g, 4.12 mmol, 4.0 eq), compound 203 (0.56 g, 1.03 mmol, 1.0 eq) and HATU (1.17 g, 3.09 mmol, 3.0 eq) were added in sequence. After stirring over the ice water bath for 1 hour, 100 mL of water was added, and a solid precipitated out. The solid was collected by filtration and washed with water, dissolved in DCM, dried over anhydrous sodium sulfate, filtered and concentrated. The residue was dissolved in small amount of DCM, loaded on a silica gel column, and eluted 0-10% MeOH/DCM to give 0.60 g of light yellow foam (yield 65%). MS-ESI (m/z): calcd. for C₄₆H₆₂N₄O₁₄ [M+H]⁺ 895.43; found, 895.40.

### Example 197. Synthesis of (20R,21R)-20,21-bis(((benzyloxy)carbonyl)amino)-3,19,22-trioxo-1-phenyl-2,6,9,12,15-pentaoxa-18,23-diazaheptacosan-27-oic acid (205)

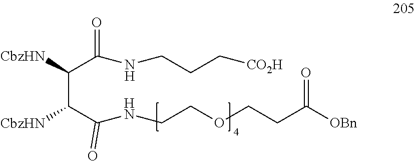

Compound 204 (0.60 g, 0.67 mmol, 1.0 eq) was dissolved in 5 mL of DCM, and stirred with 5 mL of TFA at r.t. for 3 h, and completion of the reaction was monitored by LCMS. DCM was removed and the residue was co-evaporated with DCM for three times, placed on high vacuum pump. The crude product was dissolved in a small amount of DCM and loaded on a silica gel column, and then eluted with 15-20% MeOH/DCM. Fractions were combined and concentrated to give 0.34 g of white foam (yield 60%). MS-ESI (m/z): calcd. for C₄₂H₅₄N₄O₁₄ [M+H]⁺ 839.36; found, 839.45.

### Example 198. Synthesis of 1-benzyl 25-(perfluorophenyl) (18R,19R)-18,19-bis(((benzyl-oxy)carbonyl)amino)-17,20-dioxo-4,7,10,13-tetraoxa-16,21-diazapentacosanedioate (206)

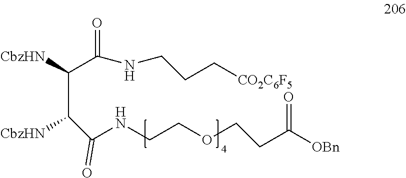

Compound 205 (0.34 g, 0.40 mmol, 1.0 eq) was dissolved in 10 mL DCM, to which pentafluorophenol (0.081 g, 0.44 mmol, 1.1 eq) and EDC (0.38 g, 2.0 mmol, 5.0 eq) were added. The reaction was stirred at r.t. overnight and then washed (2×10 mL) and brine (20 mL), dried over anhydrous sodium sulfate, filtered and concentrated. The crude product was used directly in the next step. MS-ESI (m/z): calcd. for C₄₈H₅₃ F₅N₄O₁₄ [M+H]⁺ 1561.6; found, 1561.6.

### Example 199. Synthesis of Compound 207

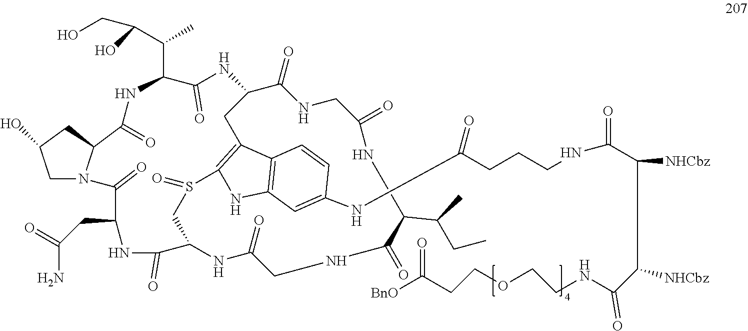

The crude product from the previous step (0.40 mmol, 1.0 eq) was dissolved in 10 mL DMF, cooled over ice water bath. To which compound 183 (0.39 g, 0.4 mmol, 1.0 eq) and DIPEA (0.15 g, 1.2 mmol, 3.0 eq) were added in sequence. After stirring over the ice water bath for 1 hour, the reaction was concentrated, and re-dissolved in a small amount of DCM, loaded on a silica gel column and eluted with 0-20% MeOH/DCM to give a colorless oil (0.40 g, 58% yield). MS-ESI (m/z): calcd. for C₈₁H₁₀₇N₁₅O₂₆S [M+2H]²⁺: 869.86; found, 869.96.

### Example 200. Synthesis of Compound 208

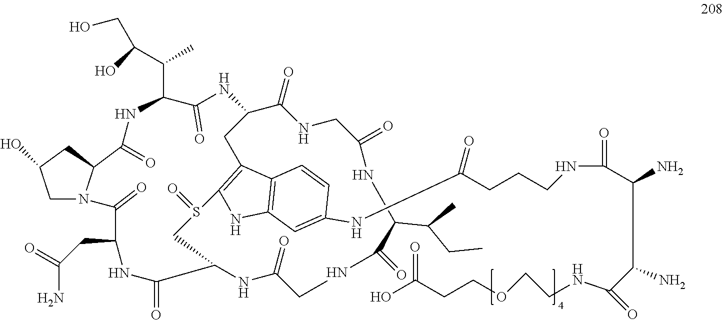

Compound 207 (0.40 g, 0.23 mmol, 1.0 eq) was dissolved in 5 mL methanol, dry palladium carbon (0.1 g, 10% wt) was added and the reaction flask was evacuated and back-filled with H2 for three times. The reaction mixture was stirred under H2 overnight, filtered and filtrate concentrated to give 0.32 g of crude material, which was directly used for the next reaction. MS-ESI (m/z): calcd. for C₅₈H₈₉N₁₅O₂₂S [M+2H]²⁺ 690.80; found, 690.85.

### Example 201. Synthesis of Compound 209

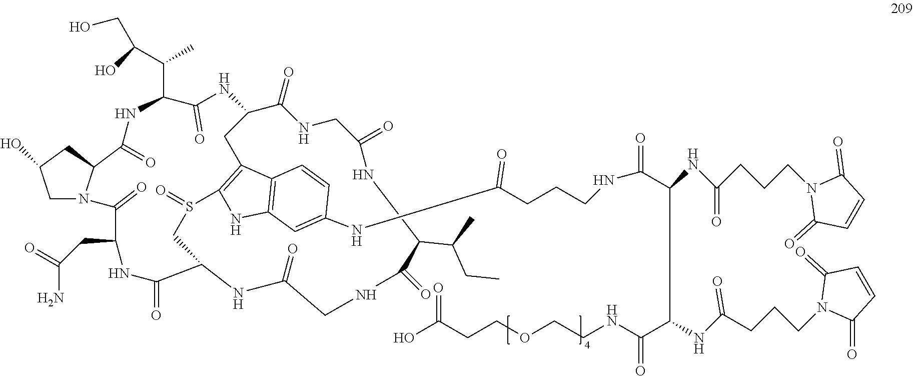

The crude product from the previous step (0.32 g, 0.23 mmol, 1.0 eq) was dissolved in 2 mL of ethanol, 0.2 mL of 0.1M NaH₂PO₄. N-(4-maleimidobutyryloxy) succinimide (0.19 g, 0.69 mmol, 3.0 eq) was added and the reaction was stirred at r.t. overnight, and then concentrated and re-dissolved in DCM, dried over anhydrous sodium sulfate, filtered and concentrated. The residue was dissolved in a small amount of DCM, and loaded on silica gel column, eluted with 0-20% MeOH/DCM to give a colorless oil (0.13 g, 33% yield). MS-ESI (m/z): calcd. for C₇₄H₁₀₃N₁₇O₂₈S [M+2H]²⁺ 855.84; found, 855.86.

### Example 202. Synthesis of di-tert-butyl (6S,13S)-9,10-bis(((benzyloxy)carbonyl) amino)-5,8,11,14-tetraoxo-6,13-bis(4-(((2,2,2-trichloroethoxy)carbonyl)amino)butyl)-4,7,12,15-tetraazaoctadecanedioate (211)

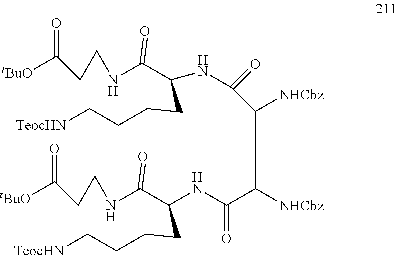

To a solution of tert-butyl (S)-3-(2-amino-6-(((2,2,2-trichloroethoxy)carbonyl) amino)hexanamido)propanoate (6.88 g, 14.4 mmol) and 2,3-bis(((benzyloxy)carbonyl) amino)succinic acid (5.00 g, 12.0 mmol) in DMA (60 mL), EDC·HCl (2.76 g, 14.4 mmol) and DIPEA (4.7 mL, 26.4 mmol) were added. The reaction mixture was stirred at r.t. overnight, then diluted with 150 mL dichloromethane and poured over 100 mL of water in a separatory funnel. The organic phase was separated, washed with brine (2×50 mL), dried over anhydrous sodium sulfate, filtered and concentrated. The residue was purified by column chromatography (10-80% ethyl acetate/petroleum ether) to afford the title compound 211 (13.0 g, 85% yield). MS-ESI (m/z): calcd. for C₅₂H₇₂Cl₆N₈O₁₆ [M+H]⁺ 638.16; found, 638.18.

### Example 203. Synthesis of di-tert-butyl (65,13S)-9,10-diamino-5,8,11,14-tetraoxo-6,13-bis(4-(((2,2,2-trichloroethoxy)carbonyl)amino)butyl)-4,7,12,15-tetraazaoctadecanedioate (212)

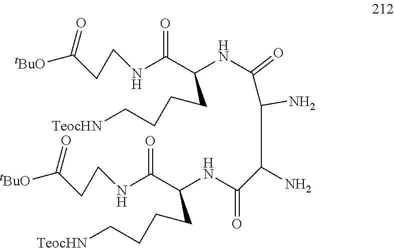

To a solution of compound 211 (12.4 g, 9.72 mmol) in methanol (50 mL) was added Pd/C (10 wt %, 0.10 g) in a hydrogenation bottle. After the bottle was evacuated and back-filled with hydrogen three times, the mixture was shaken for 2 h, filtered through Celite (filter aid), and the filtrate was concentrated to afford compound 212 (9.47 g, 97% yield) as a colorless oil. MS-ESI (m/z): calcd. for C₃₆H₆₀Cl₆N₈O₁₂ [M+H]⁺ 1007.25; found, 1007.82.

### Example 204. Synthesis of di-tert-butyl (6S,13S)-9,10-bis(3-(2,5-dioxo-2,5-dihydro-1H-pyrrol-1-yl)propanamido)-5,8,11,14-tetraoxo-6,13-bis(4-(((2,2,2-trichloroethoxy)carbonyl)amino)butyl)-4,7,12,15-tetraazaoctadecanedioate (213)

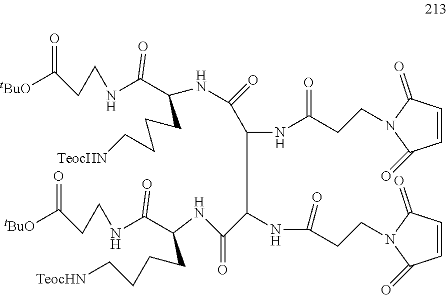

To a solution of compound 210 (9.47 g, 9.40 mmol) in dichloromethane (50 mL), 3-(2,5-dioxo-2,5-dihydro-1H-pyrrol-1-yl)propanoic acid (1.91 g, 11.3 mmol) and EDC·HCl (2.17 g, 11.3 mmol) were added, followed by DIPEA (4.0 mL, 23.5 mmol). The reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-80% ethyl acetate/petroleum ether) to give a colorless oil (9.49 g, 77% yield). MS-ESI (m/z): calcd. for C₅₀H₇₀Cl₆N₁₀O₁₈ [M+2H]²⁺ 655.15; found, 655.10.

### Example 205. Synthesis of (6S,13S)-9,10-bis(3-(2,5-dioxo-2,5-dihydro-1H-pyrrol-1-yl)propanamido)-5,8,11,14-tetraoxo-6,13-bis(4-(((2,2,2-trichloroethoxy)carbonyl) amino)butyl)-4,7,12,15-tetraazaoctadecanedioic acid (214)

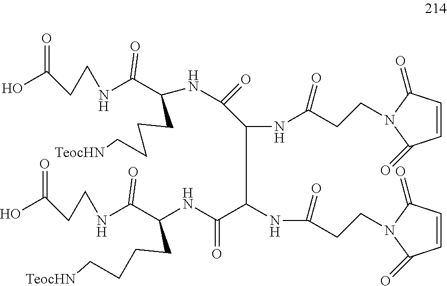

A solution of compound 213 (9.49 g, 7.60 mmol) in THF (15 mL) was treated with 4 N HCl (2 mL) at 0° C. for 30 min then concentrated and loaded on a short silica gel column and eluted with 0-15% methanol/dichloromethane to give a colorless oil (8.50 g, 93% yield). MS-ESI (m/z): calcd. for C₄₂H₅₄Cl₆N₁₀O₁₈ [M+2H]²⁺ 599.08; found, 599.10.

### Example 206. Synthesis of Compound 215

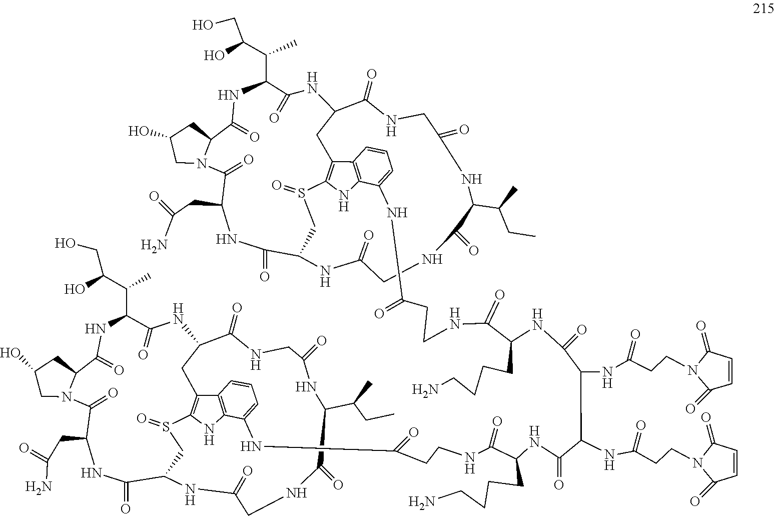 [SMILES: CC[C@H](C)[C@@H]1NC(=O)CNC(=O)C2Cc3c([nH]c4c(NC(=O)CCNC(=O)[C@H](CCCCN)NC(=O)C(NC(=O)CCN5C(=O)C=CC5=O)C(NC(=O)CCN5C(=O)C=CC5=O)C(=O)N[C@@H](CCCCN)C(=O)NCCC(=O)Nc5cccc6c7c([nH]c56)S(=O)C[C@@H]5NC(=O)CNC(=O)[C@H]([C@@H](C)CC)NC(=O)CNC(=O)[C@H](C7)NC(=O)[C@H]([C@@H](C)[C@@H](O)CO)NC(=O)[C@@H]6C[C@@H](O)CN6C(=O)[C@H](CC(N)=O)NC5=O)cccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

To a solution of compound 183 (10.0 mg, 0.0109 mmol) and compound 214 (6.5 mg, 0.00545 mmol) in DMF (1 mL), TBTU (3.50 mg, 0.0109 mmol) and DIPEA (2.0 μL, 0.0109 mmol) were added and the mixture was stirred at r.t. for 2 h. After removal of DMF under high vacuum, the residue was purified by prep-HPLC (acetonitrile/water) to give a colorless oil (14.0 mg). This oil was dissolved in THF (1.0 mL) and treated with TBAF (1.0 M in THF, 35 μL) at 0° C. for 30 min, then concentrated and purified by a short silica gel column (0-10% methanol/dichloromethane) to afford a colorless oil (10.0 mg, 34% yield). MS-ESI (m/z): calcd. for C₁₁₄H₁₅₈N₃₂O₃₈S₂ [M+3H]³⁺ 883.36; found, 883.36.

### Example 207. Synthesis of tert-butyl ((S)-37-(4-(2, 5-dioxo-2, 5-dihydro-1H-pyrrol-1-yl)butanamido)-31-oxo-2, 5, 8, 11, 14, 17, 20, 23, 26, 29-decaoxa-32-azaoctatriacontan-38-oyl)-L-alanyl-L-alanyl-L-alaninate (216)

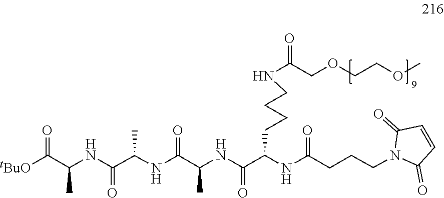

Compound 134 (1.70 g, 1.92 mmol) and 4-maleimidobutanoic acid (0.35 g, 1.92 mmol) were dissolved in 20 mL of DCM, and EDC·HCl (0.74 g) was added. After stirring at 0° C. for 2 hours the reaction was diluted with 50 mL of DCM, washed with 50 mL of water, 50 mL of brine, concentrated to give a white solid (1.9 g, 94% yield). MS-ESI (m/z): calcd. for C₄₈H₈₅N₆O₉[M+H]⁺ 1049.58; found, 1049.58.

### Example 208. Synthesis of ((S)-37-(4-(2, 5-dioxo-2, 5-dihydro-1H-pyrrol-1-yl)butanamido)-31-oxo-2, 5, 8, 11, 14, 17, 20, 23, 26, 29-decaoxa-32-azaoctatriacontan-38-oyl)-L-alanyl-L-alanyl-L-alanine (217)

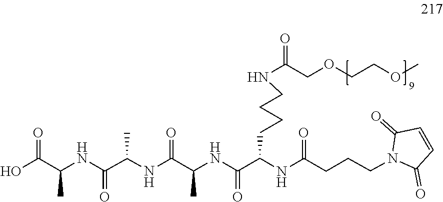

Compound 216 (1.9 g, 1.81 mmol) was dissolved in 15 mL of DCM and 15 mL of TFA. After stirring at r.t. for 1 h, the reaction was concentrated, and then purified by preparative HPLC to give a colorless liquid (1.1 g, 63% yield). MS-ESI (m/z): calcd. for C₄₄H₇₇N₆O₁₉ [M+H]⁺ 993.52; found, 993.52.

### Example 209. Synthesis of 2, 5-dioxopyrrolidin-1-yl ((S)-37-(4-(2, 5-dioxo-2, 5-dihydro-1H-pyrrol-1-yl)butanamido)-31-oxo-2, 5, 8, 11, 14, 17, 20, 23, 26, 29-decaoxa-32-azaoctatriacontan-38-oyl)-L-alanyl-L-alanyl-L-alaninate (218)

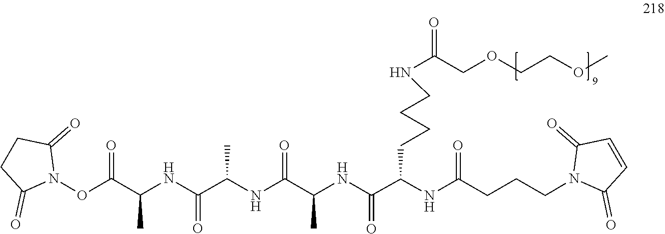

Compound 217 (1.1 g, 1.14 mmol) was dissolved in 40 mL of DCM, NHS (0.21 g, 1.82 mmol) and EDC·HCl (0.45 g, 2.35 mmol) were added. After stirring at r.t. for 1 h, the reaction was washed with 50 mL of water, 50 mL of brine, dried over anhydrous sodium sulfate, filtered, concentrated to give a white solid (1.0 g, 100% yield). MS-ESI (m/z): calcd. for C₄₈H₈₀N₇O₂₁ [M+H]⁺ 1090.53; found, 1090.55.

### Example 210. Synthesis of (2S, 4R)-4-((tert-butoxycarbonyl)amino)-5-(3-((37S, 40S, 43S, 46S)-37-(4-(2, 5-dioxo-2, 5-dihydro-1H-pyrrol-1-yl)butanamido)-40, 43, 46-trimethyl-31, 38, 41, 44-tetraoxo-2, 5, 8, 11, 14, 17, 20, 23, 26, 29-decaoxa-32, 39, 42, 45-tetraazaheptatetracontan-47-amido)-4-hydroxyphenyl)-2-methylpentanoic acid (219)

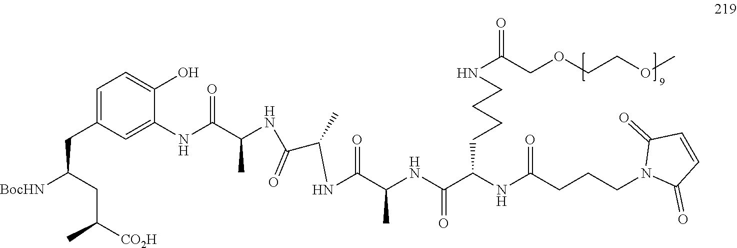

Compound 218 (1.00 g, 0.91 mmol) and compound 77 (0.43 g, 1.27 mmol) were dissolved in 40 mL of THF. After stirring at 50° C. overnight, the reaction was concentrated, and the residue was purified by preparative HPLC to give a colorless liquid (0.50 g, 33% yield). MS-ESI (m/z): calcd. for C₆₁H₁₀₁N₈O₂₃ [M+H]⁺ 1313.69; found, 1313.69.

### Example 211. Synthesis of (2S, 4R)-4-amino-5-(3-((37S, 40S, 43S, 46S)-37-(4-(2, 5-dioxo-2, 5-dihydro-1H-pyrrol-1-yl)butanamido)-40, 43, 46-trimethyl-31, 38, 41, 44-tetraoxo-2, 5, 8, 11, 14, 17, 20, 23, 26, 29-decaoxa-32, 39, 42, 45-tetraazaheptatetracontan-47-amido)-4-hydroxyphenyl)-2-methylpentanoic acid (220)

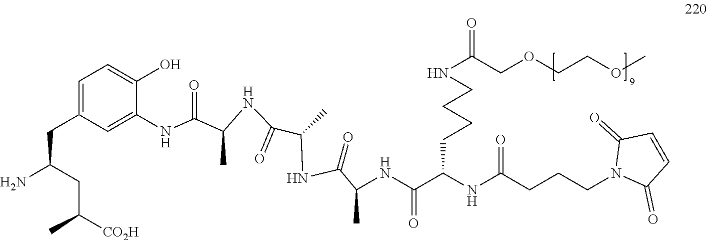

Compound 219 (286 mg, 0.22 mmol) was dissolved in 10 mL of DCM and 10 mL of TFA. After stirring at r.t. for 1 h, the reaction was concentrated to give a light yellow liquid (563 mg, 100% yield). MS-ESI (m/z): calcd. for C₅₆H₉₃N₈O₂₁ [M+H]⁺ 1213.64; found, 1213.64.

### Example 212. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S, 43S, 46S)-37-(4-(2, 5-dioxo-2, 5-dihydro-1H-pyrrol-1-yl)butanamido)-40, 43, 46-trimethyl-31, 38, 41, 44-tetraoxo-2, 5, 8, 11, 14, 17, 20, 23, 26, 29-decaoxa-32, 39, 42, 45-tetraazaheptatetracontan-47-amido)-4-hydroxyphenyl)-2-methylpentanoic acid (221)

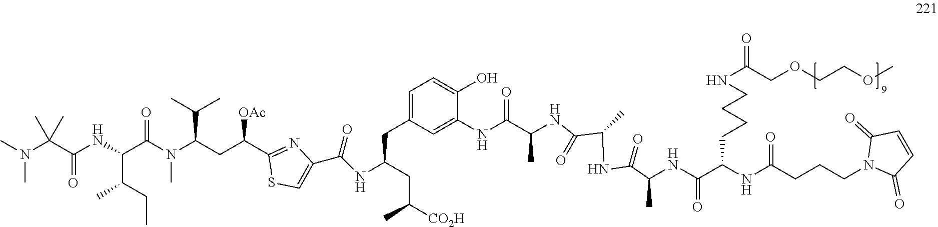

Compound 220 (260.0 mg, 0.21 mmol) and Tub-1 (178 mg, 0.26 mmol) were dissolved in 10 mL of DMF, N,N-diisopropylethylamine (499 mg, 3.86 mmol) was added until pH-9. After stirring at 0° C. for 0.5 h, the reaction was concentrated, purified by preparative HPLC to give a light yellow liquid (407 mg, 100% yield). MS-ESI (m/z): calcd. for C₈₁H₁₃₃N₁₂O₂₆S [M+H]⁺ 1721.91; found, 1721.91.

### Example 213. Synthesis of tert-butyl (S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycinate (222)

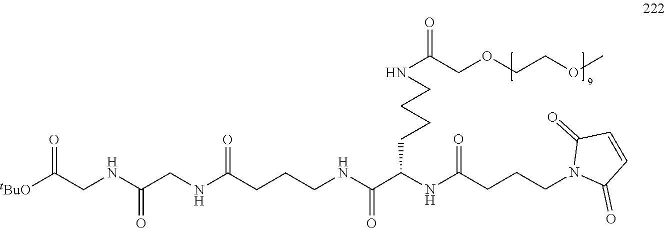

Compound 27 (6.10 g, 0.007 mol) was dissolved in DCM (80 mL), pentafluorophenol (1.83 g, 0.010 mol) and N, N′-diisopropylcarbodiimide (1.67 g, 0.013 mol) were added. After stirring for 1 h, 4-maleimidobutanoic acid (1.22 g, 0.007 mol) and N,N-diisopropylethylamine (2.2 mL, 0.013 mol) were added. The reaction was stirred for 1 h, washed with 100 mL of water, brine, dried over anhydrous sodium sulfate, filtered, concentrated and purified by silica gel column (DCM/MeOH=100/12), to give compound 222 (6.85 g, 100% yield). MS-ESI (m/z):calcd. for C₄₇H₈₂N₆O₁₉ [M+H]⁺ 1035.56; found, 1035.61.

### Example 214. Synthesis of (S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycine (223)

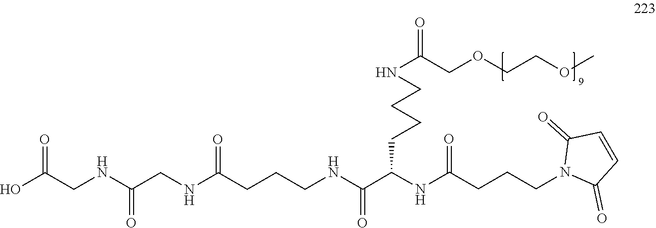

Compound 222 (6.85 g, 0.007 mol) was dissolved in DCM (30 mL) and TFA (15 mL). The reaction was stirred overnight, concentrated, and purified by preparative HPLC to give compound 223 (6.03 g, 93% yield). MS-ESI (m/z): calcd. for C₄₃H₇₄N₆O₁₉ [M+H]⁺ 979.50; found, 980.21.

### Example 215. Synthesis of 2, 5-dioxopyrrolidin-1-yl (S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycinate (224)

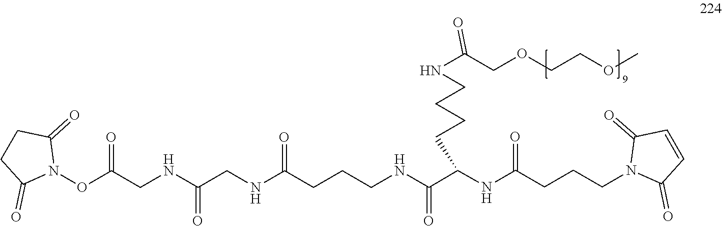

Compound 223 (3.52 g, 0.004 mol) was dissolved in DCM (40 mL), N-hydroxysuccinimide (0.54 g, 0.005 mol) and EDC·HCl (1.24 g, 0.006 mol) were added. The reaction was stirred for 1.5 hours, washed with brine, dried over anhydrous sodium sulfate, filtered and concentrated to give compound 224 (3.20 g, 83% yield). MS-ESI (m/z): calcd. for C₄₇H₇₇N₇O₂₁ [M+H]⁺ 1076.52; found, 1076.69.

### Example 216. Synthesis of (2S, 4R)-4-((tert-butoxycarbonyl)amino)-5-(3-((S)-37-(4-(2, 5-dioxo-2, 5-dihydro-1H-pyrrol-1-yl)butanamido)-31, 38, 43, 46-tetraoxo-2, 5, 8, 11, 14, 17, 20, 23, 26, 29-decaoxa-32, 39, 44, 47-tetraazanonatetracontan-49-amido)-4-hydroxyphenyl)-2-methylpentanoic acid (225)

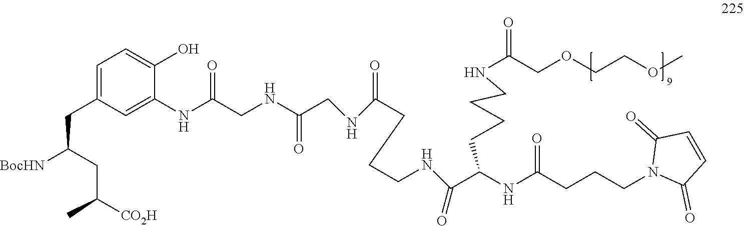

Compound 224 (3.20 g, 0.003 mol) and compound 77 (1.21 g, 0.004 mol) were dissolved in THF (30 mL), stirred at 25° C. for 2 days, concentrated and purified by preparative HPLC to give compound 225 (2.66 g, 69% yield). MS-ESI (m/z): calcd. for C₆₀H₉₈N₈O₂₃ [M+H]⁺ 1299.67; found, 1300.30.

### Example 217. Synthesis of (2S, 4R)-4-amino-5-(3-((S)-37-(4-(2, 5-dioxo-2, 5-dihydro-1H-pyrrol-1-yl)butanamido)-31, 38, 43, 46-tetraoxo-2, 5, 8, 11, 14, 17, 20, 23, 26, 29-decaoxa-32, 39, 44, 47-tetraazanonatetracontan-49-amido)-4-hydroxyphenyl)-2-methylpentanoic acid (226)

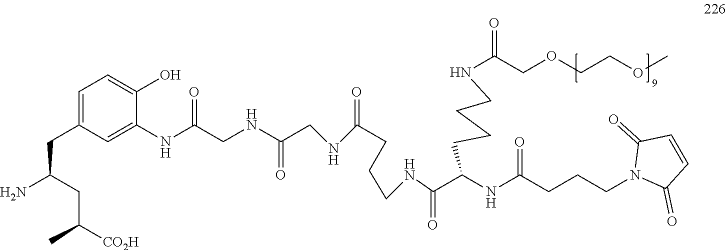

Compound 225 (2.66 g, 0.002 mol) was dissolved in DCM (10 mL) and TFA (20 mL). The reaction was stirred for 2 hours, and then concentrated. The residue was purified by preparative HPLC to give compound 226 (2.46 g, 100% yield). MS-ESI (m/z): calcd. for C₅₅H₉₀N₈O₂₁ [M+H]⁺ 1199.62; found, 1199.92.

### Example 218. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 43, 46-tetraoxo-2, 5, 8, 11, 14, 17, 20, 23, 26, 29-decaoxa-32, 39, 44, 47-tetraazanonatetracontan-49-amido)-4-hydroxyphenyl)-2-methylpentanoic acid (227)

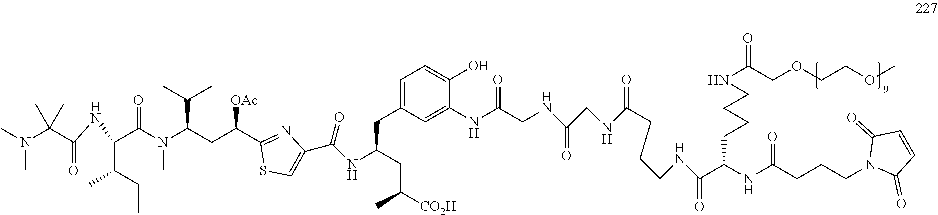

Compound 226 (2.46 g, 0.002 mol) and Tub-1 (1.71 g, 0.002 mol) were dissolved in DMF (30 mL) and N,N-diisopropylethylamine (0.339 mL, 0.002 mol) was added. The reaction was stirred for 1 h and concentrated, the residue was purified by preparative HPLC to give compound 227 (1.225 g, 35% yield). MS-ESI (m/z): calcd. for C₈₀H₁₃N₁₂O₂₆S [M+H]⁺ 1707.89; found, 1708.42.

### Example 219. Synthesis of tert-butyl (S)-(S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycylglycinate (228)

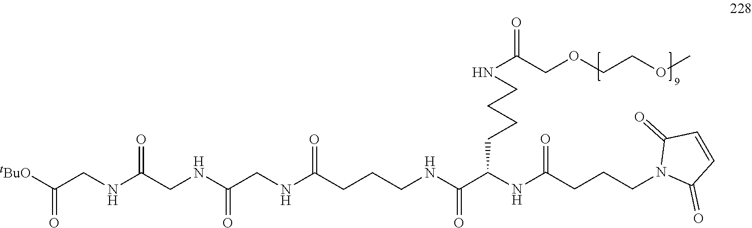

Compound 41 (1.85 g, 2.00 mmol) and 4-maleimidobutanoic acid (0.46 g, 2.50 mmol) were dissolved in 20 mL of DCM, and EDC·HCl (0.58 g, 3.00 mmol) was added. After stirring at 0° C. for 2 hours the reaction was diluted with 50 mL of DCM, washed with 50 mL of water, 50 mL of brine, concentrated to give a white solid (2.10 g, 95% yield). MS-ESI (m/z): calcd. for C₄₉H₈₅N₇O₂₀ [M+H]⁺ 1092.58; found, 1092.58.

### Example 220. Synthesis of (S)-(S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycylglycine (229)

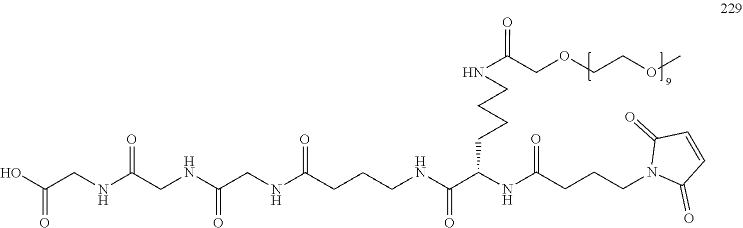

Compound 228 (2.10 g, 1.92 mmol) was dissolved in DCM (30 mL) and TFA (15 mL). The reaction was stirred overnight, concentrated, and purified by preparative HPLC to give compound 229 (1.83 g, 92% yield). MS-ESI (m/z): calcd. for C₄₅H₇₅N₇O₂₀ [M+H]⁺ 1036.52; found, 1036.55.

### Example 221. Synthesis of 2, 5-dioxopyrrolidin-1-yl (S)-(S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycylglycinate (230)

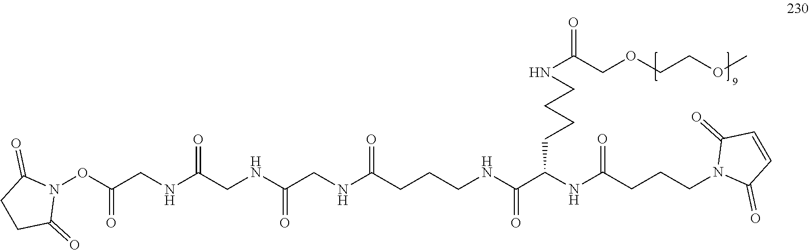

Compound 229 (500 mg, 0.483 mmol) was dissolved in DCM (10 mL), N-hydroxysuccinimide (67 mg, 0.579 mmol) and EDC·HCl (139 mg, 0.724 mmol) were added. The reaction was stirred at r.t. for 6 hours, washed with brine, dried over anhydrous sodium sulfate, filtered and concentrated to give compound 230 (546 mg, 99% yield). MS-ESI (m/z): calcd. for C₄₉H₈₀N₈O₂₂ [M+H]⁺ 1133.54; found, 1134.01.

### Example 222. Synthesis of (2S, 4R)-4-((tert-butoxycarbonyl)amino)-5-(3-((S)-37-(4-(2, 5-dioxo-2, 5-dihydro-1H-pyrrol-1-yl)butanamido)-31, 38, 43, 46, 49-pentaoxo-2, 5, 8, 11, 14, 17, 20, 23, 26, 29-decaoxa-32, 39, 44, 47, 50-pentaazadopentacontan-52-amido)-4-hydroxyphenyl)-2-methylpentanoic acid (231)

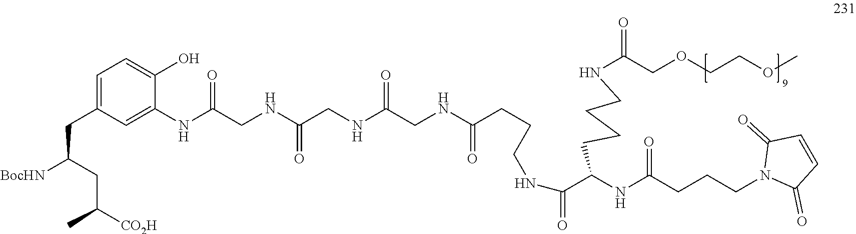

Compound 230 (360 mg, 0.318 mmol) and compound 77 (129 mg, 0.381 mmol) were dissolved in THF (15 mL) and DCM (10 mL). The reaction was stirred at r.t. overnight, concentrated and purified by preparative HPLC to give compound 231 (200 mg, 0.147 mmol, 46% yield). MS-ESI (m/z): calcd. for C₆₂H₁₀₁N₉O₂₄ [M+H]⁺ 1356.70; found, 1357.00.

### Example 223. Synthesis of (2S, 4R)-4-amino-5-(3-((S)-37-(4-(2, 5-dioxo-2, 5-dihydro-1H-pyrrol-1-yl)butanamido)-31, 38, 43, 46, 49-pentaoxo-2, 5, 8, 11, 14, 17, 20, 23, 26, 29-decaoxa-32, 39, 44, 47, 50-pentaazadopentacontan-52-amido)-4-hydroxyphenyl)-2-methylpentanoic acid (232)

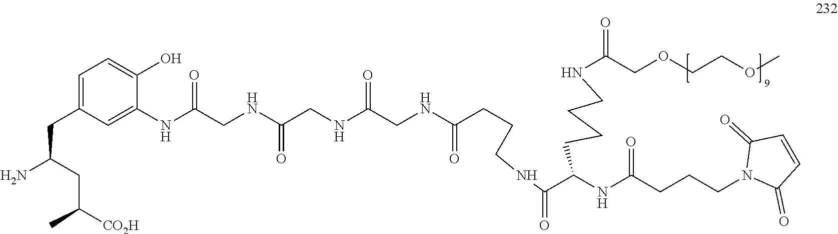

Compound 231 (200 mg, 0.147 mmol) was dissolved in DCM (4 mL) and TFA (2 mL). After stirring for 2 hours, the reaction solution was concentrated to give 232 (185 mg, 100% yield). MS-ESI (m/z): calcd. for C₅₇H₉₃N₉O₂₂ [M+H]⁺ 1256.64; found, 1257.07.

### Example 224. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 43, 46, 49-pentaoxo-2, 5, 8, 11, 14, 17, 20, 23, 26, 29-decaoxa-32, 39, 44, 47, 50-pentaazadopentacontan-52-amido)-4-hydroxyphenyl)-2-methylpentanoic acid (233)

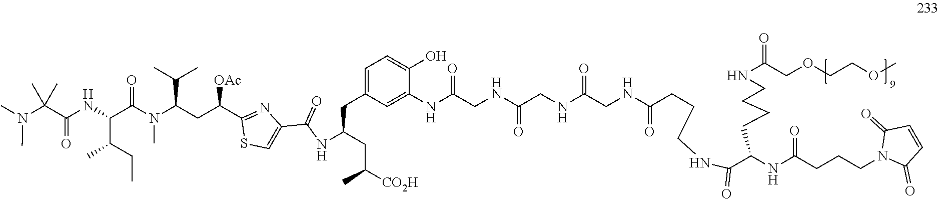

Compound 232 (185 mg, 0.147 mmol) and Tub-1 (102 mg, 0.147 mmol) were dissolved in DMF (2 mL) and N,N-diisopropylethylamine (0.097 mL, 0.589 mmol) was added. The reaction was stirred overnight and directly purified by preparative HPLC to give compound 233 (107 mg, 41% yield). MS-ESI (m/z): calcd. for C₈₂H₁₃₃N₁₃O₂₇S [M+H]⁺ 1764.92; found, 1765.19.

### Example 225. Synthesis of tert-butyl (S)-2-((4-(37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)benzyl)oxy)acetate (236)

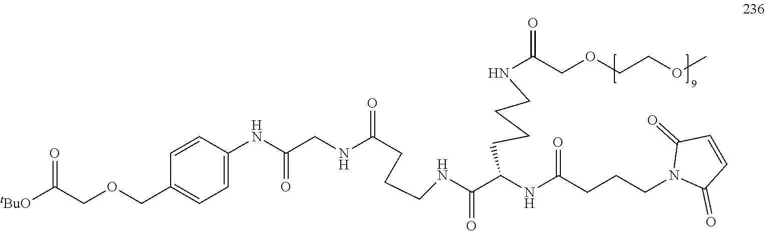

Compound 234 (8.40 g, 9.11 mmol) and compound 235 (2.59 g, 10.9 mmol) were dissolved in 60 mL of DCM. EDC·HCl (3.48 g, 18.2 mmol) and N,N-diisopropylethylamine (3.49 g, 18.2 mmol) were added. The reaction was stirred at 0° C. for 1 h, washed with 100 mL of water, and the aqueous phase was extracted with 50 mL of DCM. The organic phases were combined, dried over anhydrous sodium sulfate, filtered and concentrated. The crude was purified by a silica gel column, eluted with ethyl acetate/petroleum ether and by preparative HPLC, to give compound 236 as brown liquid (3.0 g, 29% yield). MS-ESI (m/z): calcd. for C₅₄H₈₉N₆O₂₀ [M+H]⁺ 1141.61; found, 1141.61.

### Example 226. Synthesis of (S)-2-((4-(37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)benzyl)oxy)acetic acid (237)

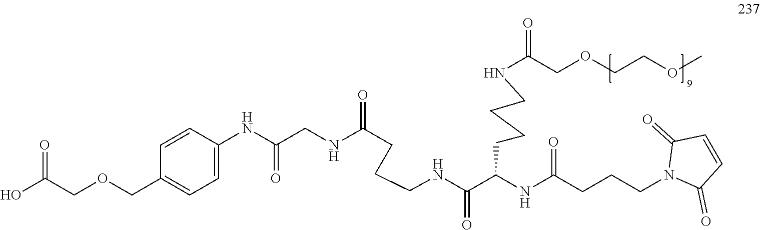

Compound 236 (3.0 g, 2.63 mmol) was dissolved in 30 mL of formic acid and 30 mL of DCM. The reaction was stirred at 40-50° C. for 3 h, concentrated and purified by preparative HPLC to give 237 as a colorless liquid (1.2 g, 42% yield). MS-ESI (m/z): calcd. for C₅₀H₈₁N₆O₂₀ [M+H]⁺1085.54; found, 1085.54.

### Example 227. Synthesis of 2, 5-dioxopyrrolidin-1-yl (S)-2-((4-(37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)benzyl)oxy)acetate (238)

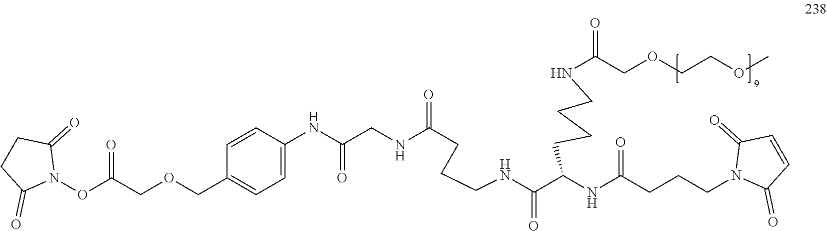

Compound 237 (450 mg, 0.41 mmol) and N-hydroxysuccinimide (71 mg, 0.617 mmol) were dissolved in 10 mL of DCM, EDC·HCl (126 mg, 0.657 mmol) was then added. After stirring at r.t. for 3 h, the reaction was washed with 30 mL of water, 30 mL of brine, dried over anhydrous sodium sulfate, filtered and concentrated to give 238 as a colorless liquid (0.71 g, 100% yield). MS-ESI (m/z): calcd. for C₅₄H₈₄N₇O₂₂ [M+H]⁺ 1182.56; found, 1182.56.

### Example 228. Synthesis of (2S, 4R)-4-((tert-butoxycarbonyl)amino)-5-(3-(2-((4-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)benzyl)oxy)acetamido)-4-hydroxyphenyl)-2-methylpentanoic acid (239)

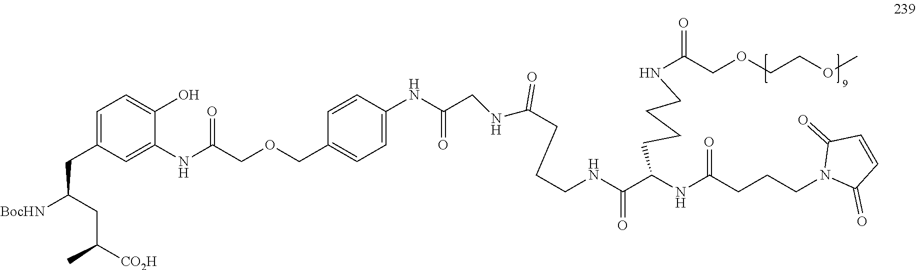

Compound 238 (710 mg, 0.41 mmol) and compound 77 (174 mg, 0.51 mmol) were dissolved in 10 mL of THF and stirred at 50-55° C. overnight. The reaction was then concentrated, diluted with 30 mL of DCM, washed with 30 mL of water, 30 mL of brine, dried over anhydrous sodium sulfate, filtered, concentrated and purified by preparative HPLC to give a colorless liquid (114 mg, 20% yield). MS-ESI (m/z): calcd. for C₆₇H₁₀₅N₈O₂₄ [M+H]⁺ 1405.72; found, 1405.72.

### Example 229. Synthesis of (2S, 4R)-4-amino-5-(3-(2-((4-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)benzyl)oxy)acetamido)-4-hydroxyphenyl)-2-methylpentanoic acid (240)

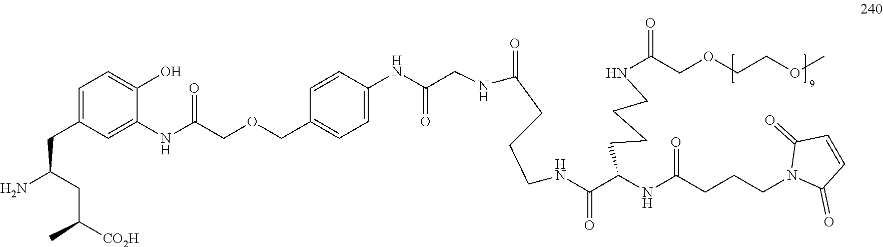

Compound 239 (100 mg, 0.07 mmol) was dissolved in 10 mL of DCM and 3 mL of TFA. After stirring at 0° C. for 2 hours, the reaction was concentrated to give a colorless liquid (0.50 g, 100% yield). MS-ESI (m/z): calcd. for C₆₂H₉₇N₈O₂₂ [M+H]⁺ 13.5.66; found, 1305.66.

### Example 230. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-(2-((4-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)benzyl)oxy)acetamido)-4-hydroxyphenyl)-2-methylpentanoic acid (241)

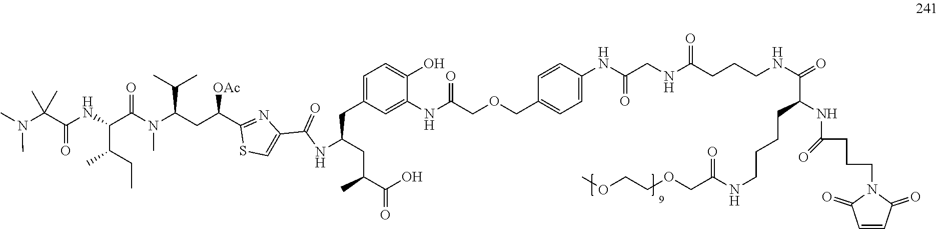

Compound 239 (92.8 mg, 0.07 mmol) and Tub-1 (60.2 mg, 0.08 mmol) were dissolved in 10 mL of DMF, N,N-diisopropylethylamine (445 mg, 3.44 mmol) was added. After stirring at 0° C. for 0.5 h and r.t. for 2 hours, the reaction was concentrated and purified by preparative HPLC, to give a light yellow solid (75 mg, 58% yield). MS-ESI (m/z): calcd. for C₈₇H₁₃₇N₁₂O₂₇S [M+H]⁺ 1813.94; found, 1813.94.

### Example 231. Synthesis of tert-butyl (S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-oate (244)

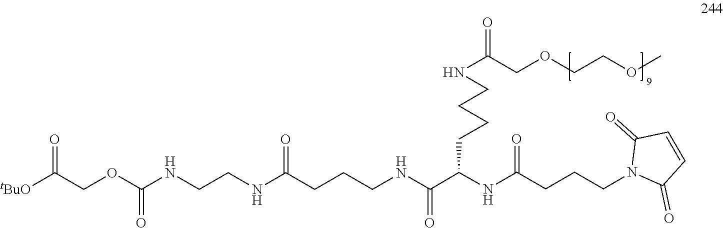

Compound 243 (2.80 g, 12.8 mmol) and compound 242 (4.44 g, 5.13 mmol) were dissolved in DCM (50 mL), HATU (2.14 g, 5.64 mmol) and N,N-diisopropylethylamine (2.5 mL, 15.4 mmol) were added. The reaction was stirred for 3 hours, and then concentrated, the residue was purified by silica gel column (DCM/MeOH=100/8), to give compound 244 (4.70 g, 86% yield). MS-ESI (m/z): calcd. for C₄₈H₈₄N₆O₂₀ [M+H]⁺ 1065.57; found, 1065.94.

### Example 232. Synthesis of (S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-oic acid (245)

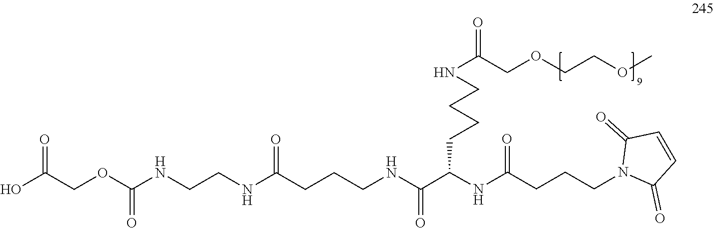

Compound 244 (4.70 g, 0.004 mol) was dissolved in DCM (40 mL) and TFA (20 mL). The reaction was stirred overnight, concentrated, and purified by preparative HPLC to give compound 245 (1.00 g, 22% yield). MS-ESI (m/z): calcd. for C₄₄H₇₆N₆O₂₀ [M+H]⁺ 1009.51; found, 1009.72.

### Example 233. Synthesis of (2S, 4R)-4-((tert-butoxycarbonyl)amino)-5-(3-((S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-amido)-4-hydroxyphenyl)-2-methylpentanoic acid (246)

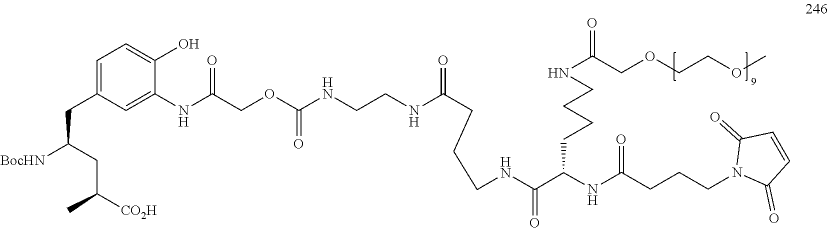

Compound 245 (343 mg, 0.340 mmol) was dissolved in DCM (10 mL), and N-hydroxysuccinimide (47 mg, 0.408 mmol) and EDC·HCl (98 mg, 0.510 mmol) were added. The reaction was stirred for 4 hours, washed with brine (5 mL), dried over anhydrous sodium sulfate, filtered and concentrated. The residue was dissolved in 20 mL of anhydrous THF, and compound 77 (138 mg, 0.408 mmol) was added. The reaction was stirred overnight, concentrated and purified by preparative HPLC to give compound 246 (177 mg, 39% yield). MS-ESI (m/z): calcd. for C₆₁H₁₀₀N₈O₂₄ [M+H]⁺ 1329.69; found, 1329.98.

### Example 234. Synthesis of (2S, 4R)-4-amino-5-(3-((S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-amido)-4-hydroxyphenyl)-2-methylpentanoic acid (247)

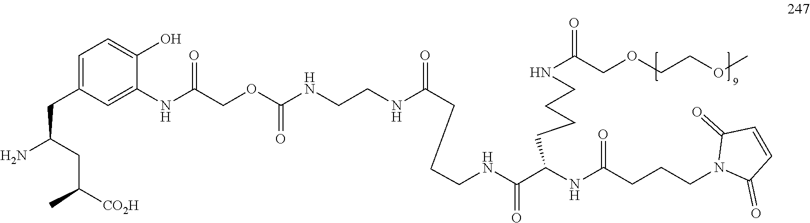

Compound 246 (177 mg, 0.133 mol) was dissolved in DCM (4 mL) and TFA (2 mL). The reaction was stirred for 1 h, and concentrated to give compound 247 (0.16 g, 97% yield). MS-ESI (m/z): calcd. for C₅₆H₉₂N₈O₂₂ [M+H]⁺ 1229.63; found, 1231.31.

### Example 235. Synthesis of (2S, 4R)-4-(2-((6S, 9R, 11R)-6, 9-diisopropyl-2, 3, 3, 8-tetramethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-amido)-4-hydroxyphenyl)-2-methylpentanoic acid (248)

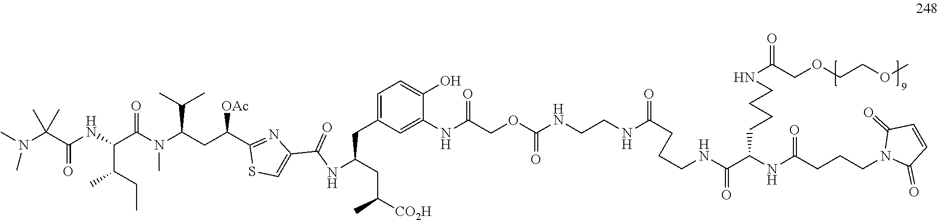

Compound 247 (160 mg, 0.130 mmol) and Tub-1 (90 mg, 0.130 mmol) were dissolved in DMF (2 mL), and N,N-diisopropylethylamine (17 mg, 0.130 mmol) was added. The reaction was stirred for 4 hours and directly purified by preparative HPLC to give compound 248 (127 mg, 56% yield). MS-ESI (m/z): calcd. for C₈₁H₁₃₂N₁₂O₂₇S [M+H]⁺ 1737.90; found, 1738.58.

### Example 236. Synthesis of tert-butyl (6S, 9S, 12S, 15S)-6-(3-methoxy-3-oxopropyl)-2, 2, 9, 12, 15-pentamethyl-4, 7, 10, 13-tetraoxo-3-oxa-5, 8, 11, 14-tetraazahexadecan-16-oate (249)

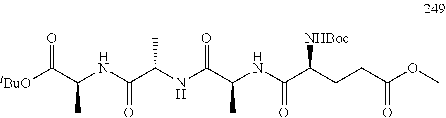 [SMILES: COC(=O)CC[C@H](NC(=O)OC(C)(C)C)C(=O)N[C@@H](C)C(=O)N[C@@H](C)C(=O)N[C@@H](C)C(=O)OC(C)(C)C]

Compound 132 (4.00 g, 0.014 mol) and Boc-Glu(OMe)-OH (3.64 g, 0.014 mol) were dissolved in DCM (100 mL), and HATU (7.94 g, 0.021 mol) and TEA (3.870 mL, 0.028 mol) were added. The reaction was stirred for 1.5 hours, and washed with brine (2×150 mL), dried over anhydrous sodium sulfate, filtered and concentrated to give compound 249 (7.39 g, 100% yield), which was directly used in the next step without further purification. MS-ESI (m/z): calcd. for C₂₄H₄₂N₄O₉ [M+H]⁺ 531.30; found, 531.26.

### Example 237. Synthesis of ((S)-2-amino-5-methoxy-5-oxopentanoyl)-L-alanyl-L-alanyl-L-alanine (250)

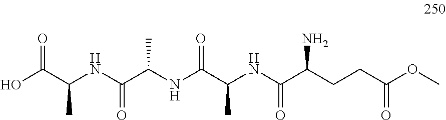 [SMILES: COC(=O)CC[C@H](N)C(=O)N[C@@H](C)C(=O)N[C@@H](C)C(=O)N[C@@H](C)C(=O)O]

Compound 249 (7.39 g, 0.014 mol) was dissolved in DCM (20 mL) and TFA (20 mL). The reaction was stirred overnight, and concentrated to give compound 250 (5.21 g, 100% yield), which was directly used in the next step without further purification. MS-ESI (m/z): calcd. for C₁₅H₂₆N₄O₇ [M+H]⁺ 375.18; found, 375.22.

### Example 238. Synthesis of ((S)-2-(4-(2, 5-dioxo-2, 5-dihydro-1H-pyrrol-1-yl)butanamido)-5-methoxy-5-oxopentanoyl)-L-alanyl-L-alanyl-L-alanine (251)

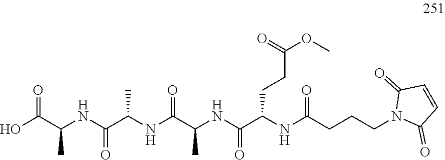 [SMILES: COC(=O)CC[C@H](NC(=O)CCCN1C(=O)C=CC1=O)C(=O)N[C@@H](C)C(=O)N[C@@H](C)C(=O)N[C@@H](C)C(=O)O]

Compound 250 (1.38 g, 0.004 mol) was dissolved in DCM (60 mL) and N,N-diisopropylethylamine (1.2 mL, 0.007 mol) was added, followed by 4-maleimidobutyric acid N-hydroxysuccinimide ester (2.06 g, 0.008 mol). After stirring for 3 hours, the reaction was extracted with water (2×100 mL), and the aqueous phase was concentrated to give a white solid (1.40 g, 70% yield). MS-ESI (m/z): calcd. for C₂₃H₃₃N₅O₁₀ [M+H]⁺ 540.22; found, 540.29.

### Example 239. Synthesis of 2, 5-dioxopyrrolidin-1-yl (2S, 5S, 8S, 11S)-16-(2, 5-dioxo-2, 5-dihydro-1H-pyrrol-1-yl)-11-(3-methoxy-3-oxopropyl)-2, 5, 8-trimethyl-4, 7, 10, 13-tetraoxo-3, 6, 9, 12-tetraazahexadecanoate (252)

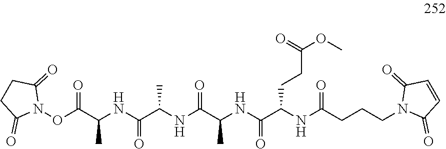 [SMILES: COC(=O)CC[C@H](NC(=O)CCCN1C(=O)C=CC1=O)C(=O)N[C@@H](C)C(=O)N[C@@H](C)C(=O)N[C@@H](C)C(=O)ON1C(=O)CCC1=O]

Compound 251 (1.40 g, 0.003 mol) was dissolved in DCM (100 mL), NHS (0.33 g, 0.003 mol) and EDC·HCl (0.99 g, 0.005 mol) were added. The reaction was stirred for 1 h, washed with 50 mL of brine, and the organic phase was dried over anhydrous sodium sulfate, filtered and concentrated to give compound 252 (1.65 g, 100% yield). MS-ESI (m/z): calcd. for C₂₇H₃₆N₆O₁₂ [M+H]⁺ 637.24; found, 637.36.

### Example 240. Synthesis of (2S, 4R)-4-((tert-butoxycarbonyl)amino)-5-(3-((6S, 9S, 12S, 15S)-6-(4-(2, 5-dioxo-2, 5-dihydro-1H-pyrrol-1-yl)butanamido)-9, 12, 15-trimethyl-3, 7, 10, 13-tetraoxo-2-oxa-8, 11, 14-triazahexadecan-16-amido)-4-hydroxyphenyl)-2-methylpentanoic acid (253)

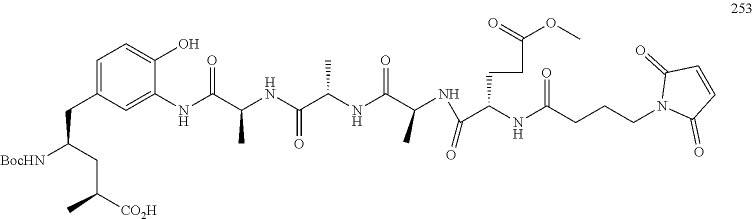 [SMILES: COC(=O)CC[C@H](NC(=O)CCCN1C(=O)C=CC1=O)C(=O)N[C@@H](C)C(=O)N[C@@H](C)C(=O)N[C@@H](C)C(=O)Nc1cc(C[C@@H](C[C@H](C)C(=O)O)NC(=O)OC(C)(C)C)ccc1O]

Compound 252 (1.65 g, 0.003 mol) and compound 77 (0.88 g, 0.003 mol) were dissolved in THF (50 mL) and reacted at 50° C. overnight. Concentration and purification by preparative HPLC gave compound 253 (0.17 g, 7.6% yield). MS-ESI (m/z): calcd. for C₄₀H₅₇N₇O₁₄ [M+H]⁺ 860.40; found, 860.44.

### Example 241. Synthesis of (2S, 4R)-4-amino-5-(3-((6S, 9S, 12S, 15S)-6-(4-(2, 5-dioxo-2, 5-dihydro-1H-pyrrol-1-yl)butanamido)-9, 12, 15-trimethyl-3, 7, 10, 13-tetraoxo-2-oxa-8, 11, 14-triazahexadecan-16-amido)-4-hydroxyphenyl)-2-methylpentanoic acid (254)

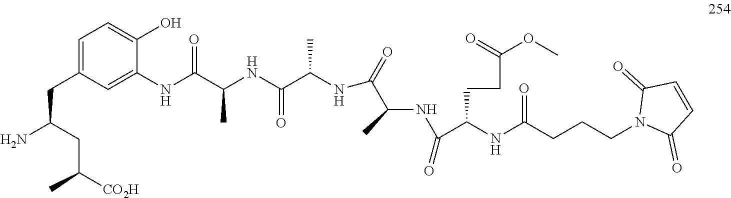 [SMILES: COC(=O)CC[C@H](NC(=O)CCCN1C(=O)C=CC1=O)C(=O)N[C@@H](C)C(=O)N[C@@H](C)C(=O)N[C@@H](C)C(=O)Nc1cc(C[C@H](N)C[C@H](C)C(=O)O)ccc1O]

Compound 253 (0.17 g, 0.198 mmol) was dissolved in DCM (4 mL) and TFA (4 mL). The reaction was stirred for 1 h, concentrated to give compound 254 (0.15 g, 100% yield). MS-ESI (m/z): calcd. for C₃₅H₄₉N₇O₁₂ [M+H]⁺ 760.34; found, 760.42.

### Example 242. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((6S, 9S, 12S, 15S)-6-(4-(2, 5-dioxo-2, 5-dihydro-1H-pyrrol-1-yl)butanamido)-9, 12, 15-trimethyl-3, 7, 10, 13-tetraoxo-2-oxa-8, 11, 14-triazahexadecan-16-amido)-4-hydroxyphenyl)-2-methylpentanoic acid (255)

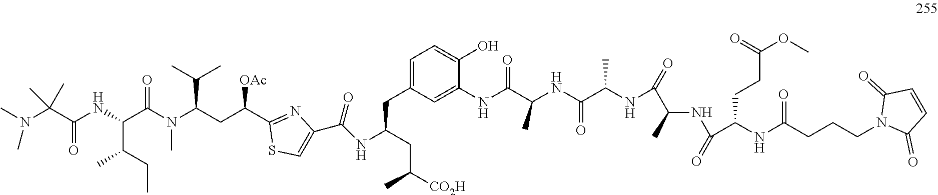 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(O)c(NC(=O)[C@H](C)NC(=O)[C@H](C)NC(=O)[C@H](C)NC(=O)[C@H](CCC(=O)OC)NC(=O)CCCN3C(=O)C=CC3=O)c2)C[C@H](C)C(=O)O)cs1)C(C)C]

Compound 254 (0.14 g, 0.20 mmol) and Tub-1 (0.15 g, 0.21 mol) were dissolved in DMF (2 mL) and N,N-diisopropylethylamine (0.098 mL, 0.001 mol) was added. The reaction was stirred for 1 h, directly purified by preparative HPLC to give compound 255 (55 mg, 22% yield). MS-ESI (m/z): calcd. for C₆₀H₈₉N₁₁O₁₇S [M+H]⁺ 1268.62; found, 1268.94.

### Example 243. Synthesis of (2S, 4R)-4-((tert-butoxycarbonyl)amino)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-2-methylpentanoic acid (256)

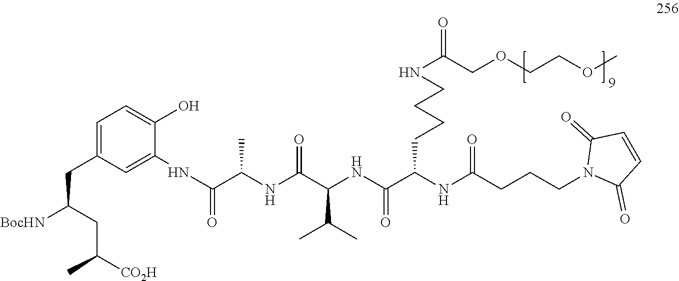

To a solution of 4-maleimidobutyric acid N-hydroxysuccinimide ester (2.6 mmol) in DCM (20 mL), compound 79 (1.95 g, 1.7 mmol) and N-methylmorpholine (0.53 g, 5.2 mmol) were added. After stirring at r.t. overnight, the reaction was washed with brine (2×20 mL), dried over anhydrous sodium sulfate, filtered and concentrated. The residue was purified by preparative HPLC to give a white solid (1.8 g, 80% yield). MS-ESI (m/z): calcd. for C₆₀H₉₉N₇O₂₂ [M+H]⁺ 1270.68; found, 1271.24.

### Example 244. Synthesis of (2S, 4R)-4-amino-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-2-methylpentanoic acid (257)

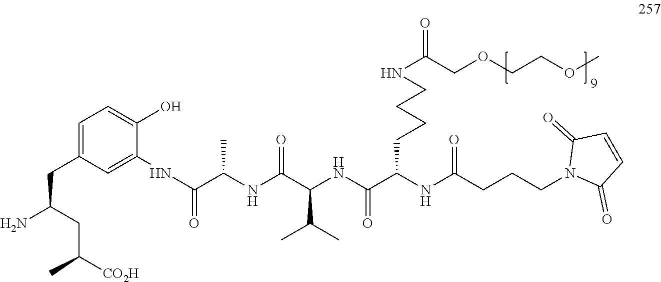

Compound 256 (1.8 g, 1.4 mmol) was dissolved in DCM (10 mL) and TFA (10 mL). The reaction was stirred at r.t. for 2 hours, concentrated and purified by preparative HPLC to give compound 257 (1.6 g, 95% yield). MS-ESI (m/z): calcd. for C₅₅H₉₁N₇O₂₀ [M+H]⁺ 1170.63; found, 1171.10.

### Example 245. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-2-methylpentanoic acid (258)

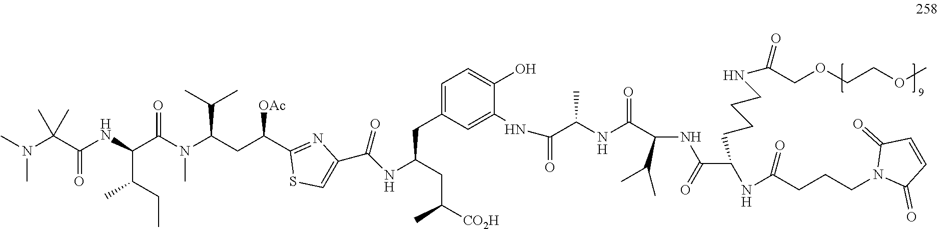

Compound 257 (520 mg, 0.44 mmol) and Tub-1 (320 mg, 0.44 mmol) were dissolved in 5 mL of DMF, N,N-diisopropylethylamine (90 mg, 0.67 mmol) was added at 0° C. After stirring at 0° C. for 2 hours, the reaction was concentrated and purified by preparative HPLC, to give an off-white solid (435 mg, 58% yield). MS-ESI (m/z): calcd. for C₈₀H₁₃₁N₁₁O₂₅S [M+H]⁺ 1678.90; found, 1679.56.

### Example 246. Synthesis of (2S, 4R)-4-amino-5-(4-hydroxy-3-nitrophenyl)-2-methylpentanoic acid (260)

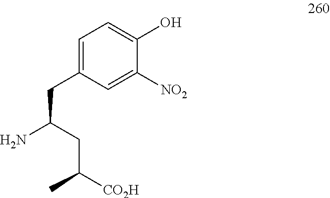 [SMILES: C[C@@H](C[C@@H](N)Cc1ccc(O)c([N+](=O)[O-])c1)C(=O)O]

Compound 259 (2.0 g, 5.43 mmol) was dissolved in methanolic HCl (4 M, 20 mL) and stirred for 3 hours, and then concentrated to give a yellow oil (1.66 g, 100% yield). MS-ESI (m/z): calcd. for C₁₂H₁₆N₂O₅ [M+H]⁺ 269.12; found, 269.32.

### Example 247. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(4-hydroxy-3-nitrophenyl)-2-methylpentanoic acid (261)

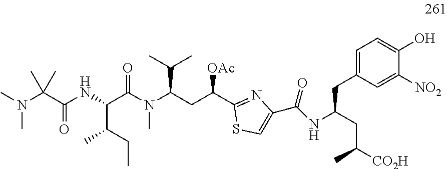 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(O)c([N+](=O)[O-])c2)C[C@H](C)C(=O)O)cs1)C(C)C]

Compound 260 (1.5 g, 5.59 mmol) and Tub-1 (3.9 g, 5.59 mmol) were dissolved in 30 mL of DMF and cooled to 0° C., N,N-diisopropylethylamine (1.5 g, 11.2 mmol) was added. After stirring at 0° C. for 2 hours, the reaction was diluted with DCM (50 mL) and washed with brine (4×50 mL), dried, filtered and concentrated and purified by preparative HPLC, to give a light yellow solid (3.5 g, 80% yield). MS-ESI (m/z): calcd. for C₃₇H₅₆N₆O₁₀S [M+H]⁺ 777.38; found, 777.58.

### Example 248. Synthesis of benzyl (2S, 4R)-5-(4-(benzyloxy)-3-nitrophenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-2-methylpentanoate (262)

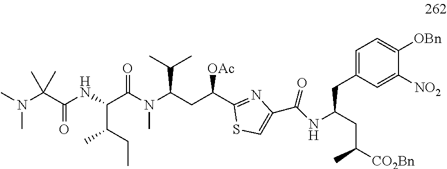 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(OCc3ccccc3)c([N+](=O)[O-])c2)C[C@H](C)C(=O)OCc2ccccc2)cs1)C(C)C]

Compound 261 (3.5 g, 4.51 mmol) and benzyl bromide (2.3 g, 13.5 mmol) were dissolved in dry DMF (60 mL) and cooled to 0° C. in an ice-water bath. Sodium hydrogen (540 mg, 60 wt %) was added in portions, and after the addition, the reaction was warmed to r.t. and stirred overnight. The solution was diluted with DCM (100 mL) and washed with brine (4×100 mL). The organic phase was dried over anhydrous sodium sulfate, filtered, and the filtrate was concentrated and purified by a silica gel column (DCM/MeOH=20:1˜10:1) to give a yellow solid (2.8 g, 65% yield). MS-ESI (m/z): calcd. for C₅₁H₆₈N₆O₁₀S [M+H]⁺957.47; found, 957.85.

### Example 249. Synthesis of benzyl (2S, 4R)-5-(3-amino-4-(benzyloxy)phenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-2-methylpentanoate (263)

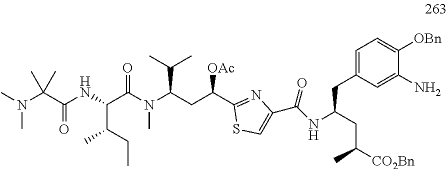 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(OCc3ccccc3)c(N)c2)C[C@H](C)C(=O)OCc2ccccc2)cs1)C(C)C]

Compound 262 (2.8 g, 2.93 mmol) was dissolved in 95% yield ethanol (50 mL), ammonium chloride (1.6 g, 29.25 mmol) and iron powder (1.6 g, 29.25 mmol) were added, and the reaction was heated under reflux overnight. The solution was diluted with DCM (100 mL) and washed with brine (200 mL). The organic phase was dried over anhydrous sodium sulfate, filtered, and the filtrate was concentrated, purified by a silica gel column (DCM/MeOH=1:0˜20:1) to give a yellow solid (2.3 g, 85% yield). MS-ESI (m/z): calcd. for C₅₁H₇₀N₆O₈S [M+H]⁺ 927.51; found, 927.65.

### Example 250. Synthesis of benzyl (2S, 4R)-5-(3-((S)-37-((((9H-fluoren-9-yl)methoxy)carbonyl)amino)-31, 38, 41-trioxo-2, 5, 8, 11, 14, 17, 20, 23, 26, 29, 44-undecaoxa-32, 39, 42-triazaheptatetracontan-47-amido)-4-(benzyloxy)phenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-2-methylpentanoate (265)

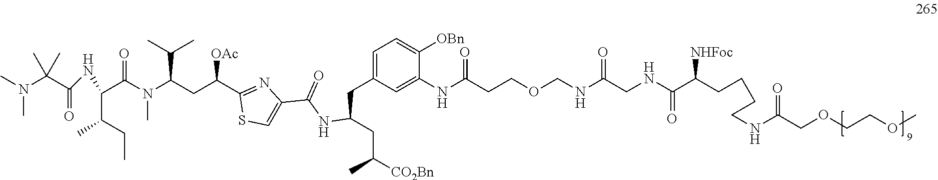

Compound 264 (750 mg, 0.75 mmol) and compound 263 (700 mg, 0.75 mmol) were dissolved in DMC (20 mL), PyBrOP (360 mg, 0.85 mmol) and N,N-diisopropylethylamine (100 mg, 0.75 mmol) were added and stirred overnight at r.t.. The reaction solution was washed with brine, dried over anhydrous sodium sulfate, filtered, and the filtrate was concentrated. The residue was purified by a silica gel column (DCM/MeOH=20:1˜10:1) to give a yellow-brown solid (420 mg, 29% yield). MS-ESI (m/z): calcd. for C₉₉H₁₄₂N₁₀O₂₅S [M+H]⁺ 1903.99; found, 1904.53.

### Example 251. Synthesis of benzyl (2S, 4R)-5-(3-((S)-37-amino-31, 38, 41-trioxo-2, 5, 8, 11, 14, 17, 20, 23, 26, 29, 44-undecaoxa-32, 39, 42-triazaheptatetracontan-47-amido)-4-(benzyloxy)phenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-2-methylpentanoate (266)

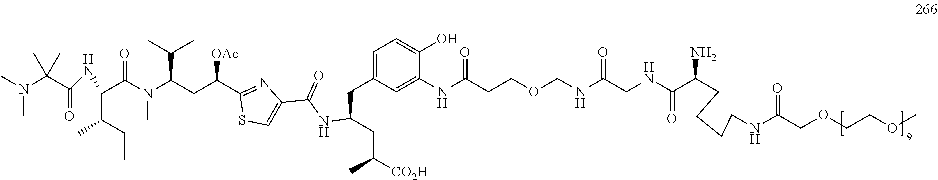

Compound 265 (420 mg, 0.22 mmol) was dissolved in isopropanol (10 mL), palladium on carbon (100 mg, 10 wt %) was added, and the reaction flask was evacuated and back-filled with hydrogen for three times and heated to 50° C. and stirred for 3 hours. The reaction mixture was filtered and the filtrate was concentrated, and purified preparative HPLC to a white solid (110 mg, 33% yield). MS-ESI (m/z): calcd. for C₇₀H₁₂MN₁₀O₂₃S [M+H]⁺ 1501.83; found, 1502.12.

### Example 252. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 41-trioxo-2, 5, 8, 11, 14, 17, 20, 23, 26, 29, 44-undecaoxa-32, 39, 42-triazaheptatetracontan-47-amido)-4-hydroxyphenyl)-2-methylpentanoic acid (267)

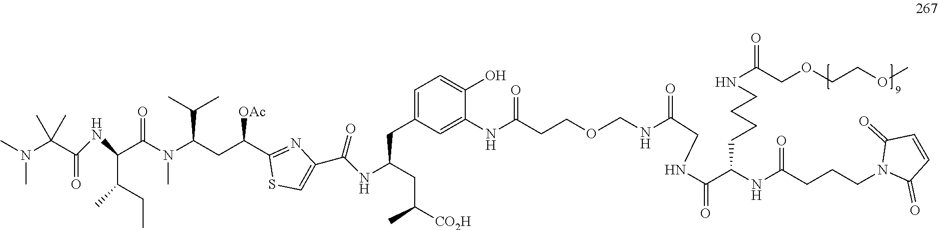

To a solution of compound 266 (110 mg, 0.07 mmol) and 4-maleimidobutyric acid N-hydroxysuccinimide ester (21 mg, 0.07 mmol) in DCM (5 mL) was added N-methylmorpholine (10 mg, 0.07 mmol). The reaction was stirred at r.t. overnight, concentrated, then purified by preparative HPLC (water/acetonitrile) to give compound 267 (15 mg, 12% yield). MS-ESI (m/z): calcd. for C₇₈H₁₂₇N₁₁O₂₆S [M+H]⁺ 1666.87; found, 1667.52.

### Example 253. Synthesis of 2, 5-dioxopyrrolidin-1-yl (S)-2-((S)-2-((((9H-fluoren-9-yl)methoxy)carbonyl)amino)-3-methylbutanamido)-5-ureidopentanoate (269)

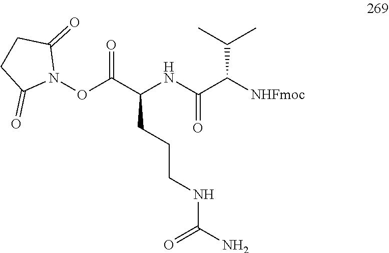

To a solution of compound 268 (4.00 g, 8.06 mmol) in 200 mL of DCM, NHS (1.39 g, 12.10 mmol) and EDC·HCl (3.09 g, 16.13 mmol) were added. The reaction was stirred for 4 hours, and diluted with 100 mL of water, and filtered to give 4.40 g of white solid (92% yield). MS-ESI (m/z): calcd. for C₃₀H₃₅N₅O₈ [M+H]⁺ 594.25; found, 594.25.

### Example 254. Synthesis of (2S, 4R)-5-(3-((S)-2-((S)-2-((((9H-fluoren-9-yl)methoxy)carbonyl)amino)-3-methylbutanamido)-5-ureidopentanamido)-4-hydroxyphenyl)-4-((tert-butoxycarbonyl)amino)-2-methylpentanoic acid (270)

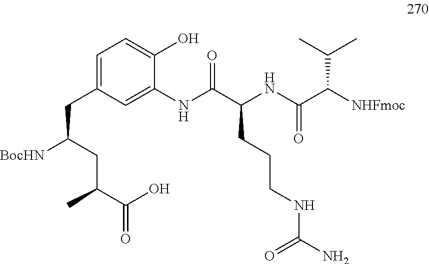

A solution of compound 269 (2.00 g, 3.37 mmol) and compound 77 (1.25 g, 3.70 mmol) in 150 mL of anhydrous THF was heated at 70° C. overnight. The reaction was concentrated and purified on a silica gel column (11% MeOH/DCM) to give a red solid (0.79 g, 29% yield). MS-ESI (m/z): calcd. for C₄₃H₅₆N₆O₁₀ [M+H]⁺ 817.41; found, 817.32.

### Example 255. Synthesis of (2S, 4R)-5-(3-((S)-2-((S)-2-amino-3-methylbutanamido)-5-ureidopentanamido)-4-hydroxyphenyl)-4-((tert-butoxycarbonyl)amino)-2-methylpentanoic acid (271)

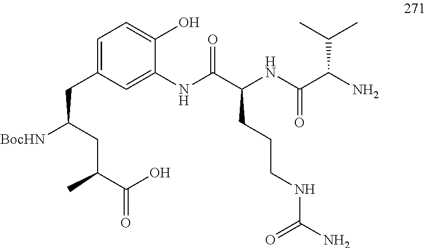 [SMILES: CC(C)[C@H](N)C(=O)N[C@@H](CCCNC(N)=O)C(=O)Nc1cc(C[C@@H](C[C@H](C)C(=O)O)NC(=O)OC(C)(C)C)ccc1O]

A solution of compound 270 (0.79 g, 0.97 mmol) in DMF wash stirred with piperidine (0.50 mL) at r.t. for 10 min., and then concentrated, co-evaporated with 5 mL of THF, to give compound 271 (0.87 g, >100% yield). MS-ESI (m/z): calcd. for C₂₈H₄₆N₆O₈ [M+H]⁺ 595.34; found, 595.35.

### Example 256. Synthesis of (2S, 4R)-5-(3-((37S, 405, 43S)-37-(((benzyloxy)carbonyl)amino)-40-isopropyl-31, 38, 41-trioxo-43-(3-ureidopropyl)-2, 5, 8, 11, 14, 17, 20, 23, 26, 29-decaoxa-32, 39, 42-triazatetratetracontan-44-amido)-4-hydroxyphenyl)-4-((tert-butoxycarbonyl)amino)-2-methylpentanoic acid (273)

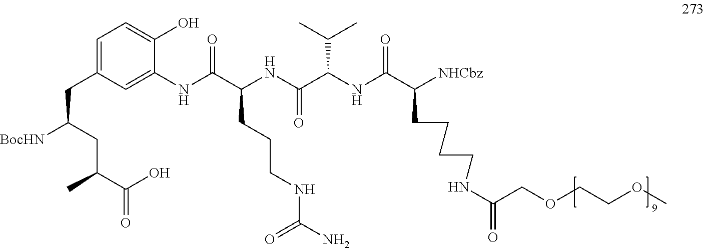

A solution of crude compound 271 (0.97 mmol) and compound 272 (1.34 mmol) in 20 mL of anhydrous THF was heated at 70° C. for 0.5 h. The reaction was concentrated and purified on a silica gel column (13% MeOH/DCM) to give compound 273 (1.13 g, 88% yield). MS-ESI (m/z): calcd. for C₆₃H₁₀₄N₈O₂₂ [M+H]⁺ 1325.73; found, 1326.28.

### Example 257. Synthesis of (2S, 4R)-5-(3-((37S, 405, 43S)-37-amino-40-isopropyl-31, 38, 41-trioxo-43-(3-ureidopropyl)-2, 5, 8, 11, 14, 17, 20, 23, 26, 29-decaoxa-32, 39, 42-triazatetratetracontan-44-amido)-4-hydroxyphenyl)-4-((tert-butoxycarbonyl)amino)-2-methylpentanoic acid (274)

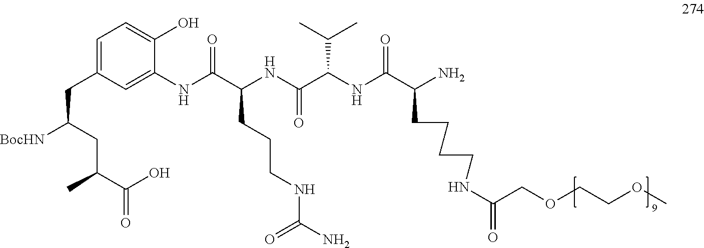

Compound 273 (1.13 g, 8.52 mmol) was dissolved in methanol (20 mL), palladium on carbon (310 mg, 10 wt %) was added, and the reaction flask was evacuated and back-filled with hydrogen for three times and stirred at 30° C. for 2 hours. The reaction mixture was filtered and the filtrate was concentrated to give compound 274 (0.77 g, 75% yield). MS-ESI (m/z): calcd. for C₅₅H₉₈N₈O₂₀ [M+H]⁺ 1191.69; found, 1192.10.

### Example 258. Synthesis of (2S, 4R)-4-((tert-butoxycarbonyl)amino)-5-(3-((37S, 40S, 43S)-37-(4-(2, 5-dioxo-2, 5-dihydro-1H-pyrrol-1-yl)butanamido)-40-isopropyl-31, 38, 41-trioxo-43-(3-ureidopropyl)-2, 5, 8, 11, 14, 17, 20, 23, 26, 29-decaoxa-32, 39, 42-triazatetratetracontan-44-amido)-4-hydroxyphenyl)-2-methylpentanoic acid (275)

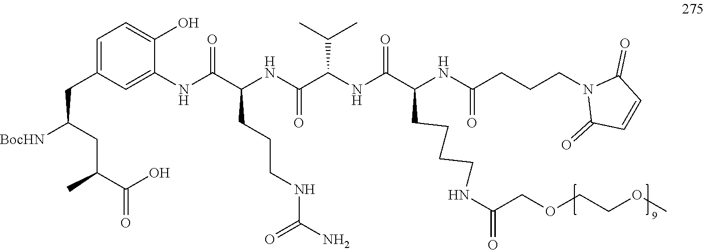

A solution of compound 274 (0.77 g, 6.44 mmol) and 4-maleimidobutyric acid N-hydroxysuccinimide ester (0.18 g, 6.55 mmol) in THF (20 mL) was stirred at 30° C. for 1 h, concentrated, then purified by preparative HPLC (water/acetonitrile) to give compound 275 (0.66 g, 76% yield). MS-ESI (m/z): calcd. for C₆₃H₁₀₅N₉O₂₃ [M+H]⁺ 1356.73; found, 1356.33.

### Example 259. Synthesis of (2S, 4R)-4-amino-5-(3-((37S, 405, 43S)-37-(4-(2, 5-dioxo-2, 5-dihydro-1H-pyrrol-1-yl)butanamido)-40-isopropyl-31, 38, 41-trioxo-43-(3-ureidopropyl)-2, 5, 8, 11, 14, 17, 20, 23, 26, 29-decaoxa-32, 39, 42-triazatetratetracontan-44-amido)-4-hydroxyphenyl)-2-methylpentanoic acid (276)

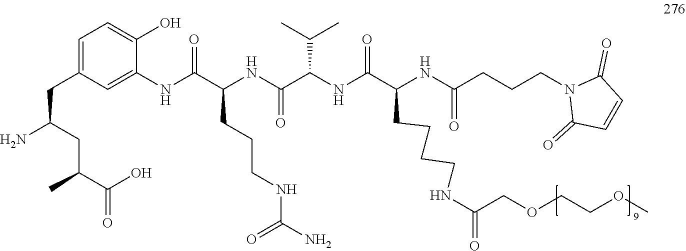

A solution of compound 276 (0.66 g, 4.86 mmol) in DCM (10 mL) was stirred with TFA (10 mL) for 40 min. and then concentrated to give a crude product (1.02 g, >100% yield). MS-ESI (m/z): calcd. for C₅₈H₉₇N₉O₂₁ [M+H]⁺ 1256.68; found, 1257.23.

### Example 260. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-31, 38, 41-trioxo-43-(3-ureidopropyl)-2, 5, 8, 11, 14, 17, 20, 23, 26, 29-decaoxa-32, 39, 42-triazatetratetracontan-44-amido)-4-hydroxyphenyl)-2-methylpentanoic acid (277)

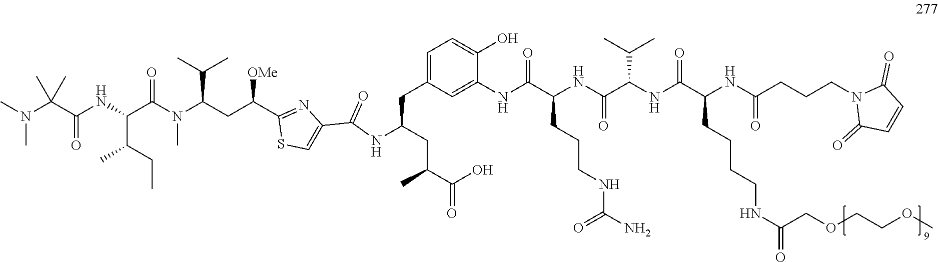

Compound 276 (0.31 g, 2.47 mmol) and Tub-1 (0.17 g, 2.49 mmol) were dissolved in 2 mL of DMF, N,N-diisopropylethylamine (163 μL, 0.99 mmol) was added and stirred for 2 hours. The reaction was concentrated and purified by preparative HPLC, to give compound 277 (343 mg, 79% yield). MS-ESI (m/z): calcd. for C₈₃H₁₃₇N₁₃O₂₆S [M+H]⁺ 1764.95; found, 1765.99.

### Example 261. Synthesis of tert-butyl (S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycinate (278)

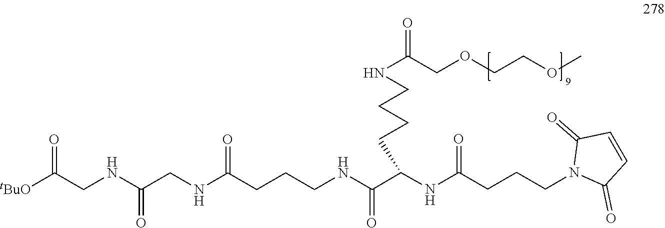

Compound 234 (1.60 g, 1.73 mmol), pentafluorophenol (0.47 g, 2.55 mmol) and N, N′-diisopropylcarbodiimide (0.43 g, 3.40 mmol) were dissolved in 20 mL of DCM, and stirred for 2 hours. H-Gly-O^{t}Bu (0.32 g, 1.91 mmol) and N,N-diisopropylethylamine (0.45 g, 3.47 mmol) were then added, and the reaction was stirred overnight, washed with 50 mL of water, 50 mL of brine, dried over anhydrous sodium sulfate, filtered, and concentrated to dryness. The crude was purified by a silica gel column to give compound 278 as a light yellow liquid (1.5 g, 83% yield). MS-ESI (m/z): calcd. for C₄₇H₈₃N₆O₁₉ [M+H]⁺ 1035.56; found, 1035.56.

### Example 262. Synthesis of (S)-(S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycylglycine (279)

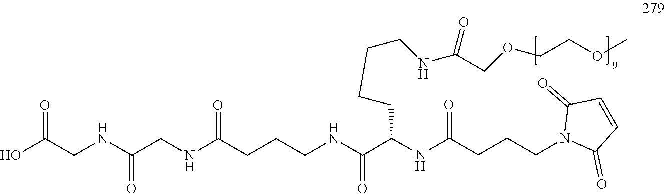

Compound 278 (1.50 g, 1.45 mmol) was dissolved in DCM (10 mL) and stirred with 20 mL of formic acid. After stirring at 40° C. for 2 hours, the reaction was concentrated to give compound 279 as a colorless liquid (1.50 g, 100% yield). MS-ESI (m/z): calcd. for C₄₃H₇₅N₆O₁₉[M+H]⁺ 979.50; found, 979.50.

### Example 263. Synthesis of tert-butyl ((2R, 4S)-1-(3-((S)-37-(4-(2, 5-dioxo-2, 5-dihydro-1H-pyrrol-1-yl)butanamido)-31, 38, 43, 46-tetraoxo-2, 5, 8, 11, 14, 17, 20, 23, 26, 29-decaoxa-32, 39, 44, 47-tetraazanonatetracontan-49-amido)-4-hydroxyphenyl)-5-((2-hydroxyethyl)amino)-4-methyl-5-oxopentan-2-yl)carbamate (281)

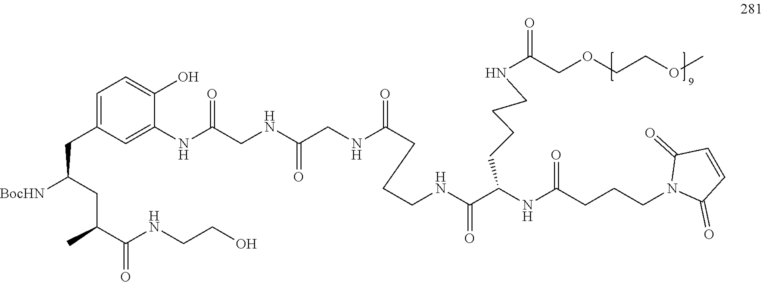

Compound 279 (500 mg, 0.51 mmol) and compound 280 (234 mg, 0.61 mmol) were dissolved in 10 mL of DCM and 3 mL of DMF, and stirred for 1 h at 0° C. The reaction mixture was concentrated and purified by a silica gel column, to give compound 281 as a light yellow liquid (0.22 g, 32% yield). MS-ESI (m/z): calcd. for C₆₂H₁₀₄N₉O₂₃ [M+H]⁺ 1342.72; found, 1342.72.

### Example 264. Synthesis of N—((S)-6-((4-((2-((2-((5-((2R, 4S)-2-amino-5-((2-hydroxyethyl)amino)-4-methyl-5-oxopentyl)-2-hydroxyphenyl)amino)-2-oxoethyl)amino)-2-oxoethyl)amino)-4-oxobutyl)amino)-5-(4-(2, 5-dioxo-2, 5-dihydro-1H-pyrrol-1-yl)butanamido)-6-oxohexyl)-2, 5, 8, 11, 14, 17, 20, 23, 26, 29-decaoxahentriacontan-31-amide (282)

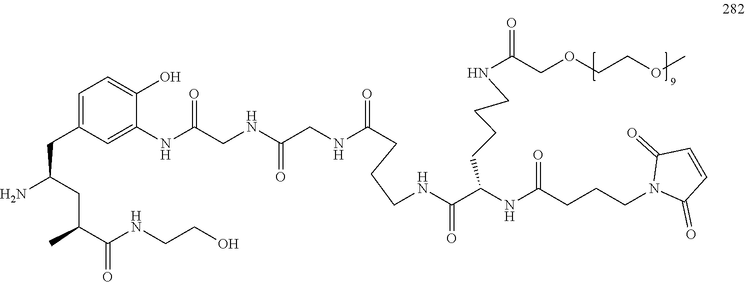

Compound 281 (0.11 g, 0.08 mmol) was dissolved in 6 mL of DCM and 2 mL of TFA. The reaction was stirred at 0° C. for 2 hours and then concentrated to give compound 282 as a light yellow liquid (0.10 g, 100% yield). MS-ESI (m/z): calcd. for C₅₇H₉₆N₉O₂₁ [M+H]⁺ 1242.66; found, 1242.66.

### Example 265. Synthesis of (1R, 3R)-3-((2S, 3S)-2-(2-(dimethylamino)-2-methylpropanamido)-N, 3-dimethylpentanamido)-1-(4-(((2R, 4S)-1-(3-((S)-37-(4-(2, 5-dioxo-2, 5-dihydro-1H-pyrrol-1-yl)butanamido)-31, 38, 43, 46-tetraoxo-2, 5, 8, 11, 14, 17, 20, 23, 26, 29-decaoxa-32, 39, 44, 47-tetraazanonatetracontan-49-amido)-4-hydroxyphenyl)-5-((2-hydroxyethyl)amino)-4-methyl-5-oxopentan-2-yl)carbamoyl)thiazol-2-yl)-4-methylpentyl acetate (283)

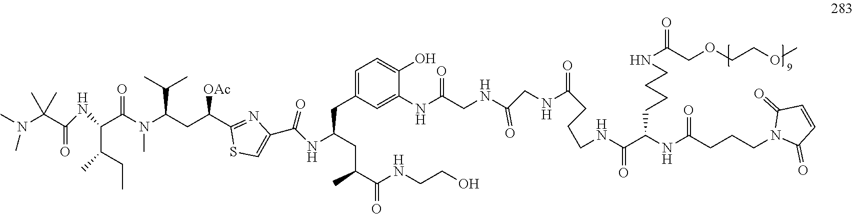

Compound 282 (0.10 g, 0.08 mmol) and Tub-1 (61 mg, 0.08 mmol) were dissolved in 5 mL of DMF, and N,N-diisopropylethylamine (125 mg, 0.96 mmol) was added at 0° C. to adjust pH to ˜9. The reaction was stirred at 0° C. for 2 hours and at r.t. for 1 h, directly purified by preparative HPLC to give compound 283 as a light yellow liquid (45 mg, 31% yield). MS-ESI (m/z): calcd. for C₈₂H₁₃₆N₁₃O₂₆S [M+H]⁺ 1750.94; found, 1750.94.

### Example 266. Synthesis of (2S, 4R)-4-(2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-2-methylpentanoic acid (284)

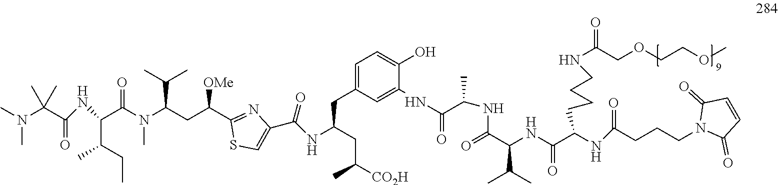

To a solution of compound 257 (300 mg, 0.256 mmol) and Tub-3 (175 mg, 0.256 mmol) in DMF (5 mL) was added N,N-diisopropylethylamine (66 mg, 0.512 mmol) at 0° C. The reaction was stirred at 0° C. for 2 hours and concentrated, then purified by preparative HPLC (water/acetonitrile) to give compound 284 (60 mg, 14% yield). MS-ESI (m/z): calcd. for C₇₉H₁₃₁N₁₁O₂₄S [M+H]⁺ 1650.90; found, 1651.50.

### Example 267. Synthesis of tert-butyl (R)-5-(3-((37S, 40S, 43S)-37-(((benzyloxy)carbonyl)amino)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-4-((tert-butoxycarbonyl)amino)pentanoate (286)

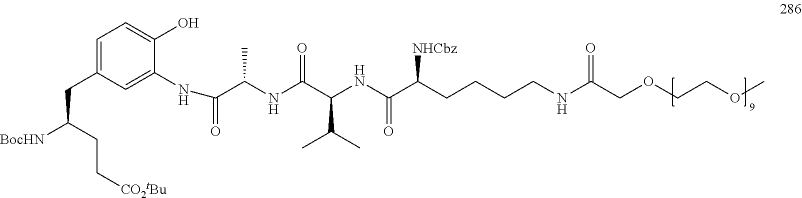

A solution of compound 76 (6.0 g, 5.9 mmol) and compound 69 (2.7 g, 7.1 mmol) in THF (100 mL) was heated at 60° C. for 20 h and then concentrated. The residue was purified on a silica gel column (DCM/MeOH=20:1) to give compound 286 as an off-white solid (7.1 g, 93% yield). MS-ESI (m/z): calcd. for C₁₆₄H₂₆₆N₂₄O₅₂S₂ [M+H]⁺ 3472.19; found, 3472.95.

### Example 268. Synthesis of tert-butyl (R)-5-(3-((37S, 40S, 43S)-37-amino-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-4-((tert-butoxycarbonyl)amino)pentanoate (287)

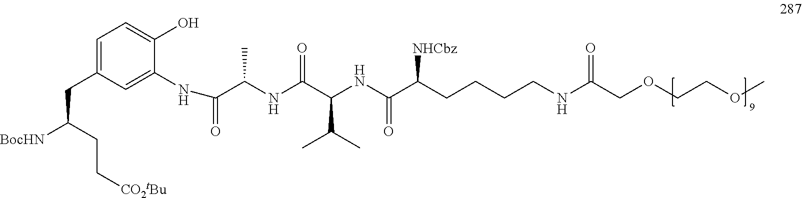

To a solution of compound 286 (7.1 g, 5.7 mmol) in isopropanol (80 mL) was added Pd/C (5 wt %, 800 mg). The reaction flask was evacuated and back-filled with hydrogen for 3 times, and then heated to 50° C. and stirred for 2.5 h. The mixture was filtered and the filtrate was concentrated to give an off-white solid (5.9 g, 93% yield). MS-ESI (m/z): calcd. for C₅₅H₉₈N₆O₁₉ [M+H]⁺ 1147.69; found, 1148.12.

### Example 269. Synthesis of tert-butyl (R)-4-((tert-butoxycarbonyl)amino)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoate (288)

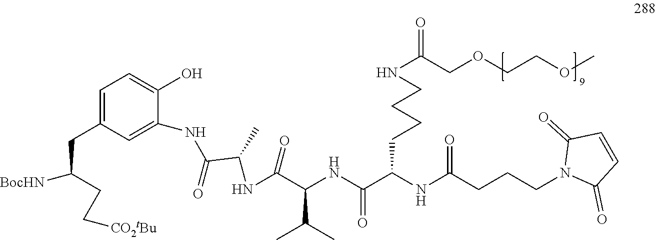

A solution of compound 287 (2.0 g, 1.7 mmol), 4-maleimidobutyric acid N-hydroxysuccinimide ester (2.6 mmol) and N-methylmorpholine (0.53 g, 5.2 mmol) in DCM (20 mL) was stirred at r.t. overnight. The mixture was then washed with brine (2×20 mL), dried over anhydrous sodium sulfate, filtered and concentrated. The residue was purified by a silica gel column (DCM/MeOH=20:1) to afford compound 288 (2.1 g, 91% yield) as a light brown oil. MS-ESI (m/z): calcd. for C₆₃H₁₀₅N₇O₂₂ [M+H]⁺ 1312.73; found, 1313.12.

### Example 270. Synthesis of (R)-4-amino-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (289)

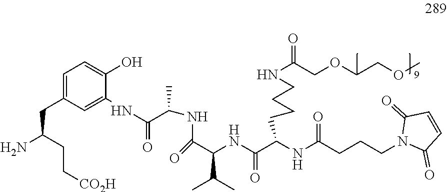

A solution of compound 288 (2 g, 1.5 mmol) in DCM (10 mL) was treated with TFA (10 mL) at r.t. for 2 hours. The solution was then concentrated and the residue was purified by preparative HPLC to give a white solid (1.4 g, 78% yield). MS-ESI (m/z): calcd. for C₅₄H₈₉N₇O₂₀ [M+H]⁺ 1156.63; found, 1157.23.

### Example 271. Synthesis of (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (290)

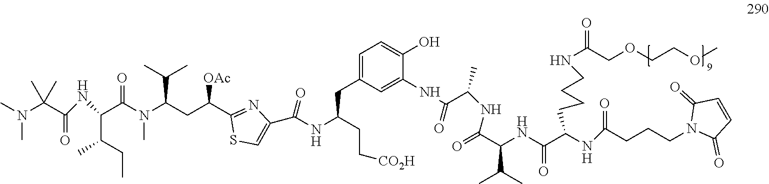

To a solution of compound 289 (1.37 g, 1.19 mmol), Tub-1 (0.90 g, 1.30 mmol) in DMF (12 mL) was added N,N-diisopropylethylamine (0.31 g, 2.37 mmol) at 0° C. The solution was stirred at 0° C. for 2 hours and then concentrated. The residue was purified by preparative HPLC to give a white solid (880 mg, 45% yield). MS-ESI (m/z): calcd. for C₇₉H₁₂₉N₁₁O₂₅S [M+H]⁺ 1664.89; found, 1665.63.

### Example 272. Synthesis of (R)-4-((tert-butoxycarbonyl)amino)-5-(3-((37S, 40S, 43S)-37-(4-(2, 5-dioxo-2, 5-dihydro-1H-pyrrol-1-yl)butanamido)-40, 43-dimethyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (293)

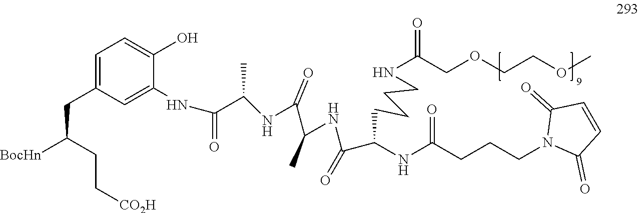

To a solution of compound 291 (470 mg, 0.51 mmol) and compound 292 (250 mg, 0.66 mmol) in DCM (10 mL) was added EDC·HCl (120 mg, 0.63 mmol) at 0° C. After stirring for 2 hours, the reaction solution was washed with water (50 mL) and brine (50 mL), dried and concentrated. The residue was purified by a silica gel column (DCM/MeOH=10:1) to give compound 293 (390 mg, 56% yield). MS-ESI (m/z): calcd. for C₅₇H₉₃N₇O₂₂ [M+H]⁺ 1229.64; found, 1229.64.

### Example 273. Synthesis of (R)-4-amino-5-(3-((37S, 40S, 43S)-37-(4-(2, 5-dioxo-2, 5-dihydro-1H-pyrrol-1-yl)butanamido)-40, 43-dimethyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (294)

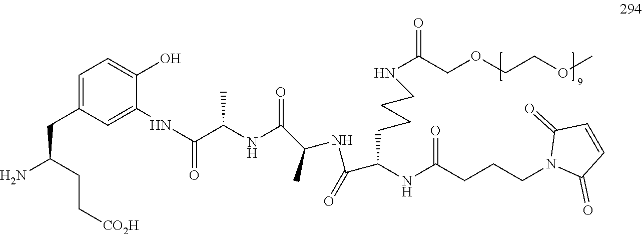

A solution of compound 293 (390 mg, 0.30 mmol) in DCM (10 mL) was treated with TFA (5 mL) at r.t. for 1 h, and then concentrated to yield compound 294 (580 mg, >100% yield), which was used directly in the next step. MS-ESI (m/z): calcd. for C₅₂H₈₅N₇O₂₀ [M+H]⁺ 1129.64; found, 1129.64.

### Example 274. Synthesis of (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S, 43S)-37-(4-(2, 5-dioxo-2, 5-dihydro-1H-pyrrol-1-yl)butanamido)-40, 43-dimethyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (295)

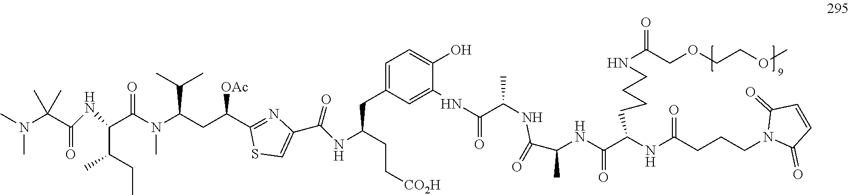

To a solution of compound 294 (580 mg, 0.43 mmol), Tub-1 (210 mg, 0.31 mmol) in DMF (5 mL) was added N,N-diisopropylethylamine (0.3 mL) at 0° C. The solution was stirred at 0° C. for 2 hours and then concentrated. The residue was purified by preparative HPLC to give a white solid (227 mg, 46% yield). MS-ESI (m/z): calcd. for C₇₇H₁₂₅N₁₁O₂₅S [M+H]⁺ 1636.86; found, 1636.86.

### Example 275. Synthesis of 2, 5-dioxopyrrolidin-1-yl ((S)-37-(4-(2, 5-dioxo-2, 5-dihydro-1H-pyrrol-1-yl)butanamido)-31-oxo-2, 5, 8, 11, 14, 17, 20, 23, 26, 29-decaoxa-32-azaoctatriacontan-38-oyl)-L-valinate (297)

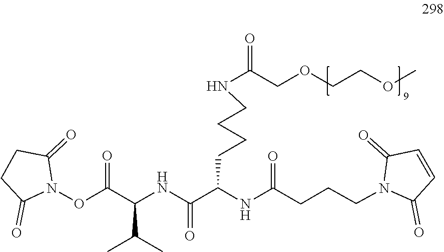

To a solution of compound 296 (879.0 mg, 1.0 mmol) in DCM (30 mL) were added NHS (126.6 mg, 1.1 mmol) and EDC·HCl (383.4 mg, 2.0 mmol). The reaction was stirred at r.t. for 2 hours and quenched with water. The organic phase was separated and washed with brine, dried over anhydrous sodium sulfate, filtered and concentrated to give the title compound (1.1 g, >100 yield). MS-ESI (m/z): calcd. for C₄₄H₇₄N₅O₁₉ [M+H]⁺ 976.49; found: 976.49.

### Example 276. Synthesis of tert-butyl (R)-4-((tert-butoxycarbonyl)amino)-5-(3-((37S, 40S)-37-(4-(2, 5-dioxo-2, 5-dihydro-1H-pyrrol-1-yl)butanamido)-40-isopropyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoate (298)

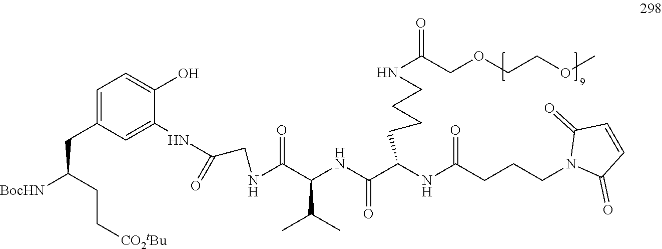

Compound 297 from previous step (550 mg, 0.5 mmol) was dissolved in THF (10 mL). Compound 85 (215.0 mg, 0.5 mmol) was added to the solution and stirred until being completely consumed. The reaction mixture was concentrated and purified by preparative HPLC, to give compound 298 (201 mg, 31% yield). MS-ESI (m/z): calcd. for C₆₂H₁₀₄N₇O₂₂ [M+H]⁺ 1298.72; found, 1298.72.

### Example 277. Synthesis of (R)-4-amino-5-(3-((37S, 40S)-37-(4-(2, 5-dioxo-2, 5-dihydro-1H-pyrrol-1-yl)butanamido)-40-isopropyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (299)

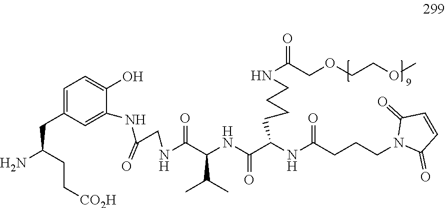

A solution of compound 298 (101 mg, 0.078 mmol) in DCM (1 mL) was treated with TFA (2 mL) at r.t. for 1 h, and then concentrated to yield compound 299 (128 mg, >100% yield), which was used directly in the next step. MS-ESI (m/z): calcd. for C₅₃H₈₈N₇O₂₀ [M+H]⁺ 1142.60; found, 1142.60.

### Example 278. Synthesis of (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S)-37-(4-(2, 5-dioxo-2, 5-dihydro-1H-pyrrol-1-yl)butanamido)-40-isopropyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (300)

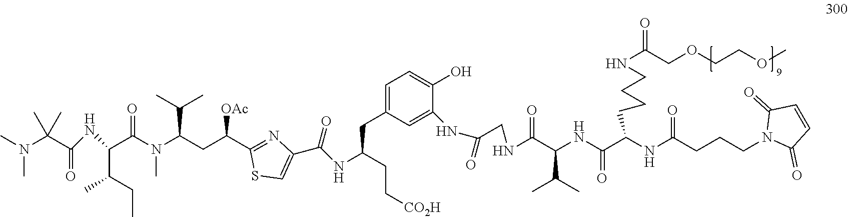

To a solution of compound 299 (70 mg, 0.06 mmol), Tub-1 (40 mg, 0.06 mmol) in DMF (2 mL) was added N,N-diisopropylethylamine (15 mg, 0.12 mmol) at 0° C. The solution was stirred at r.t. for 2 hours and then concentrated. The residue was purified by preparative HPLC to give a white solid (38 mg, 38% yield). MS-ESI (m/z): calcd. for C₇₈H₁₂₈N₁₁O₂₅S [M+H]⁺ 1650.87; found, 1650.87.

### Example 279. Synthesis of N—((S)-5-(4-(2, 5-dioxo-2, 5-dihydro-1H-pyrrol-1-yl)butanamido)-6-(((S)-1-(((S)-1-(2, 5-dioxopyrrolidin-1-yl)-1-oxopropan-2-yl)amino)-1-oxopropan-2-yl)amino)-6-oxohexyl)-2, 5, 8, 11, 14, 17, 20, 23, 26, 29-decaoxahentriacontan-31-amide (302)

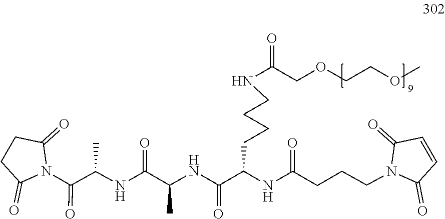

A solution of compound 301 (300 mg, 0.325 mmol), NHS (42 mg, 0.36 mmol) and EDC·HCl (95 mg, 0.50 mmol) in DCM (3 mL) was stirred at r.t. for 2 hours. The reaction mixture was diluted with DCM (20 mL) and washed with water (25 mL) and brine (25 mL), dried and concentrated to give compound 302 (280 mg, 85% yield). MS-ESI (m/z): calcd. for C₄₅H₇₂N₆O₂₀ [M+H]⁺ 1017.48; found, 1017.48.

### Example 280. Synthesis of tert-butyl (R)-4-((tert-butoxycarbonyl)amino)-5-(3-((37S, 40S, 43S, 46S)-37-(4-(2, 5-dioxo-2, 5-dihydro-1H-pyrrol-1-yl)butanamido)-40, 43, 46-trimethyl-31, 38, 41, 44-tetraoxo-2, 5, 8, 11, 14, 17, 20, 23, 26, 29-decaoxa-32, 39, 42, 45-tetraazaheptatetracontan-47-amido)-4-hydroxyphenyl)pentanoate (304)

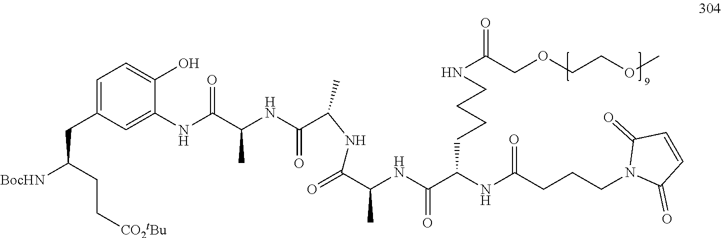

A solution of compound 302 (280 mg, 0.28 mmol) and compound 303 (200 mg, 0.44 mmol) in THF (5 mL) was stirred at r.t for 2 hours and then concentrated. The residue was purified by a silica gel column (DCM/MeOH=10:1) to give compound 304 (180 mg, 48% yield). MS-ESI (m/z): calcd. for C₆₄H₁₀₆N₈O₂₃ [M+H]⁺ 1355.74; found, 1355.74.

### Example 281. Synthesis of (R)-4-amino-5-(3-((37S, 40S, 43S, 46S)-37-(4-(2, 5-dioxo-2, 5-dihydro-1H-pyrrol-1-yl)butanamido)-40, 43, 46-trimethyl-31, 38, 41, 44-tetraoxo-2, 5, 8, 11, 14, 17, 20, 23, 26, 29-decaoxa-32, 39, 42, 45-tetraazaheptatetracontan-47-amido)-4-hydroxyphenyl)pentanoic acid (305)

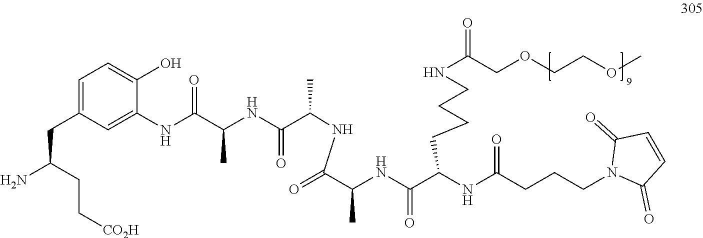

A solution of compound 307 (180 mg, 0.13 mmol) in DCM (6 mL) was treated with TFA (3 mL) at r.t. for 1 h, and then concentrated to give compound 305 (300 mg, >100% yield). MS-ESI (m/z): calcd. for C₅₅H₉₀N₈O₂₁ [M+H]⁺ 1199.62; found, 1199.62.

### Example 282. Synthesis of (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((37S, 40S, 435, 46S)-37-(4-(2, 5-dioxo-2, 5-dihydro-1H-pyrrol-1-yl)butanamido)-40, 43, 46-trimethyl-31, 38, 41, 44-tetraoxo-2, 5, 8, 11, 14, 17, 20, 23, 26, 29-decaoxa-32, 39, 42, 45-tetraazaheptatetracontan-47-amido)-4-hydroxyphenyl)pentanoic acid (306)

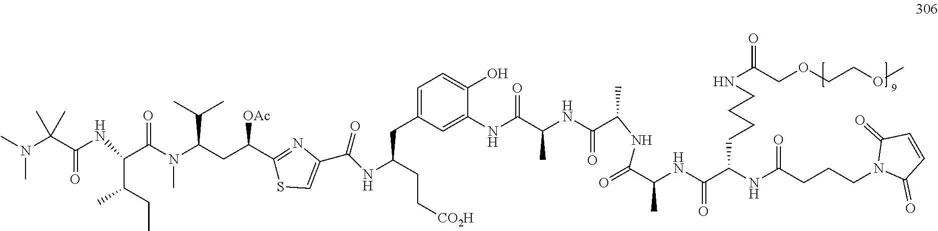

To a solution of compound 305 (160 mg, 0.13 mmol), Tub-1 (100 mg, 0.14 mmol) in DMF (5 mL) was added N,N-diisopropylethylamine (0.3 mmol) at 0° C. The solution was warmed to r.t. and stirred for 1 h and then concentrated. The residue was purified by preparative HPLC to give a white solid (58 mg, 25% yield). MS-ESI (m/z): calcd. for C₈₀H₁₃N₁₂O₂₆S [M+H]⁺ 1707.89; found, 1707.89.

### Example 283. Synthesis of methyl (S)-5-(((S)-1-(((S)-1-(((S)-1-((5-((R)-5-(tert-butoxy)-2-((tert-butoxycarbonyl)amino)-5-oxopentyl)-2-hydroxyphenyl)amino)-1-oxopropan-2-yl)amino)-1-oxopropan-2-yl)amino)-1-oxopropan-2-yl)amino)-4-(4-(2, 5-dioxo-2, 5-dihydro-1H-pyrrol-1-yl)butanamido)-5-oxopentanoate (307)

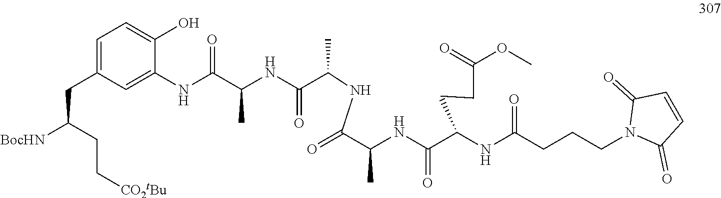 [SMILES: COC(=O)CC[C@H](NC(=O)CCCN1C(=O)C=CC1=O)C(=O)N[C@@H](C)C(=O)N[C@@H](C)C(=O)N[C@@H](C)C(=O)Nc1cc(C[C@@H](CCC(=O)OC(C)(C)C)NC(=O)OC(C)(C)C)ccc1O]

To a solution of compound 251 (0.28 g, 0.5 mmol) and compound 69 (0.20 g, 0.5 mmol) in DMF (2 mL) were added HATU (0.30 g, 0.8 mmol) and N,N-diisopropylethylamine (130 μL, 0.8 mmol). The reaction was stirred at r.t. for 10 min and directly purified by preparative HPLC to give a white solid (324 mg, 69% yield). MS-ESI (m/z): calcd. for C₄₃H₆₃N₇O₁₄ [M+H]⁺ 902.44; found, 902.81.

### Example 284. Synthesis of (R)-4-amino-5-(3-((6S, 9S, 12S, 15S)-6-(4-(2, 5-dioxo-2, 5-dihydro-1H-pyrrol-1-yl)butanamido)-9, 12, 15-trimethyl-3, 7, 10, 13-tetraoxo-2-oxa-8, 11, 14-triazahexadecan-16-amido)-4-hydroxyphenyl)pentanoic acid (308)

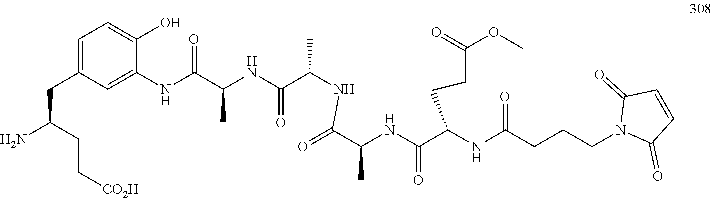 [SMILES: COC(=O)CC[C@H](NC(=O)CCCN1C(=O)C=CC1=O)C(=O)N[C@@H](C)C(=O)N[C@@H](C)C(=O)N[C@@H](C)C(=O)Nc1cc(C[C@H](N)CCC(=O)O)ccc1O]

A solution of compound 307 (0.32 g, 0.36 mmol) in DCM (10 mL) was treated with TFA (10 mL) at r.t. for 1 h, and then concentrated to give compound 308 (0.27 g, >100 yield). MS-ESI (m/z): calcd. for C₃₄H₄₇N₇O₁₂ [M+H]⁺ 746.33; found, 746.67.

### Example 285. Synthesis of (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((6S, 9S, 12S, 15S)-6-(4-(2, 5-dioxo-2, 5-dihydro-1H-pyrrol-1-yl)butanamido)-9, 12, 15-trimethyl-3, 7, 10, 13-tetraoxo-2-oxa-8, 11, 14-triazahexadecan-16-amido)-4-hydroxyphenyl)pentanoic acid (309)

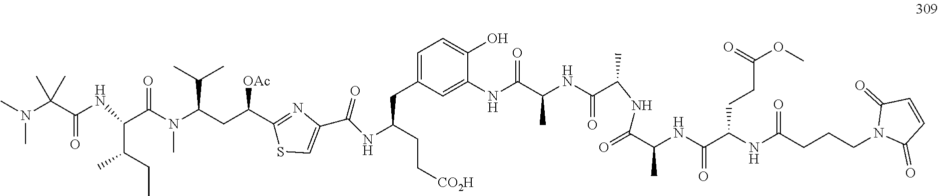 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)[C@H](C)NC(=O)[C@H](C)NC(=O)[C@H](C)NC(=O)[C@H](CCC(=O)OC)NC(=O)CCCN3C(=O)C=CC3=O)c2)cs1)C(C)C]

To a solution of compound 308 (0.27 g, 0.36 mmol), Tub-1 (0.25 g, 0.36 mmol) in DMF (2 mL) was added N,N-diisopropylethylamine (180 μL, 1.09 mmol). The solution was stirred at r.t. for 30 min and then concentrated. The residue was purified by preparative HPLC to give a white solid (367 mg, 59% yield). MS-ESI (m/z): calcd. for C₅₉H₈₇N₁₁O₁₇S [M+H]⁺ 1254.60; found, 1255.32.

### Example 286. Synthesis of (1R, 3R)-1-(4-(((R)-5-amino-1-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-5-oxopentan-2-yl)carbamoyl)thiazol-2-yl)-3-((2S, 3S)-2-(2-(dimethylamino)-2-methylpropanamido)-N, 3-dimethylpentanamido)-4-methylpentyl acetate (310)

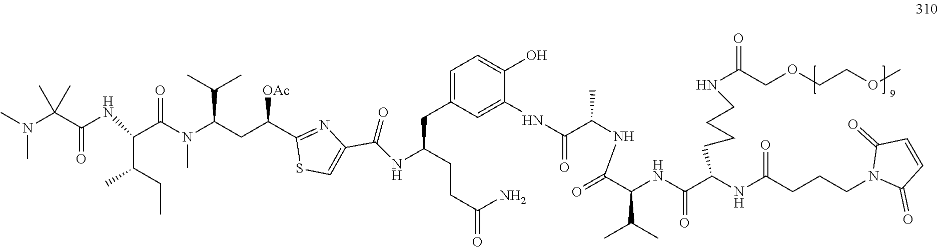

To a solution of compound 290 (300 mg, 0.2 mmol) in DCM (10 mL) were added NHS (31 mg, 0.27 mmol) and EDC·HCl (52 mg, 0.27 mmol) at 0° C. The reaction was stirred at 0° C. for 1 h and ammonium chloride (15 mg, 0.4 mmol) and N-methylmorpholine (55 mg, 0.54 mmol) were added. The reaction mixture was warmed to r.t. and stirred overnight. LC-MS indicated complete conversion of the intermediate and the crude product was then directly purified by preparative HPLC to give compound 310 (140 mg, 47% yield). MS-ESI (m/z): calcd. for C₇₉H₁₃₀N₁₂O₂₄S [M+H]⁺ 1663.90; found, 1664.93.

### Example 287. Synthesis of (1R, 3R)-3-((2S, 3S)-2-(2-(dimethylamino)-2-methylpropanamido)-N, 3-dimethylpentanamido)-1-(4-(((R)-1-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-5-hydrazineyl-5-oxopentan-2-yl)carbamoyl)thiazol-2-yl)-4-methylpentyl acetate (312)

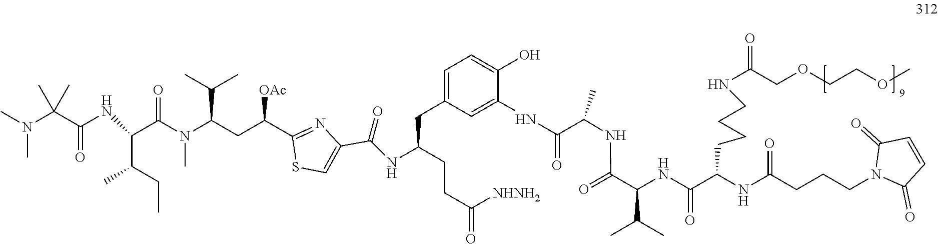

To a solution of compound 290 (300 mg, 0.2 mmol) in DCM (10 mL) were added NHS (42 mg, 0.36 mmol) and EDC·HCl (70 mg, 0.36 mmol) at 0° C. The reaction was stirred at 0° C. for 1 h and hydrazine (20 mg, 0.36 mmol) and N,N-diisopropylethylamine (70 mg, 0.54 mmol) were added. The reaction mixture was warmed to r.t. and stirred overnight. LC-MS indicated complete conversion of the intermediate and the crude product was then directly purified by preparative HPLC to give compound 312 (100 mg, 33% yield). MS-ESI (m/z): calcd. for C₇₉H₁₃₁N₁₃O₂₄S [M+H]⁺ 1678.92; found, 1678.90.

### Example 288. Synthesis of (S, E)-37-((4-((2-(tert-butoxy)-2-oxoethyl)amino)-4-oxobutyl)carbamoyl)-31, 39, 44-trioxo-2, 5, 8, 11, 14, 17, 20, 23, 26, 29-decaoxa-32, 38, 43-triazaheptatetracont-45-en-47-oic acid (314)

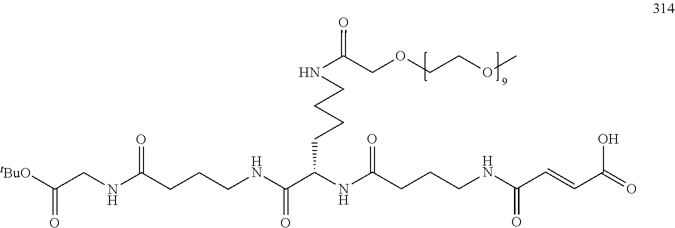

A mixture of compound 313 (1.3 g, 0.001 mol) in acetonitrile (20 mL) and sodium carbonate solution (10 mL) was stirred overnight. The organic solvent was removed and 30 mL of water was added to the solution. After adjusting pH to weak acidic with diluted hydrochloric acid, the solution was extracted with DCM (5×30 mL). The combined organic phase was dried over anhydrous sodium sulfate, filtered and concentrated to give compound 314 (1.30 g, 98% yield). MS-ESI (m/z): calcd. for C₄₅H₈₁N₅O₁₉ [M+H]⁺ 996.55; found, 996.65.

### Example 289. Synthesis of 1-(tert-butyl) 20-methyl (S, E)-4, 9, 12, 17-tetraoxo-10-(31-oxo-2, 5, 8, 11, 14, 17, 20, 23, 26, 29-decaoxa-32-azahexatriacontan-36-yl)-3, 8, 11, 16-tetraazaicos-18-enedioate (315)

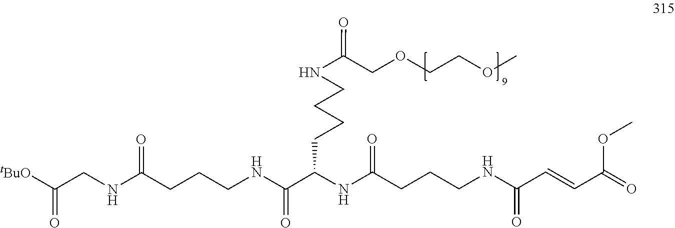

To a solution of compound 314 (1.30 g, 0.001 mol) in methanol (15 mL) was added thionyl chloride (0.095 mL, 0.001 mol) dropwise over an ice-water bath. After the bath was removed, the reaction was stirred at r.t. overnight, concentrated and purified by preparative HPLC to give compound 315 (474 mg, 36% yield). MS-ESI (m/z): calcd. for C₄₆H₈₃N₅O₁₉ [M+H]⁺ 1010.57; found, 1010.61.

### Example 290. Synthesis of (S, E)-(37-(4-(4-methoxy-4-oxobut-2-enamido)butanamido)-31, 38-dioxo-2, 5, 8, 11, 14, 17, 20, 23, 26, 29-decaoxa-32, 39-diazatritetracontan-43-oyl)glycine (316)

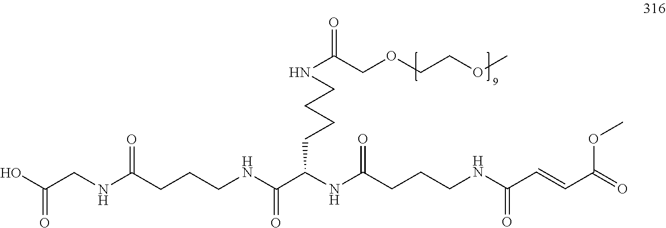

A solution of compound 315 (474 mg, 0.469 mmol) in DCM (10 mL) was treated with TFA (5 mL) at r.t. for 4 hours, concentrated and purified by preparative HPLC to give compound 316 (213 mg, 47.33% yield). MS-ESI (m/z): calcd. for C₄₂H₇₅N₅O₁₉ [M+H]⁺ 954.51; found, 954.87.

### Example 291. Synthesis of methyl (S, E)-37-((4-((2-((5-((R)-5-(tert-butoxy)-2-((tert-butoxycarbonyl)amino)-5-oxopentyl)-2-hydroxyphenyl)amino)-2-oxoethyl)amino)-4-oxobutyl)carbamoyl)-31, 39, 44-trioxo-2, 5, 8, 11, 14, 17, 20, 23, 26, 29-decaoxa-32, 38, 43-triazaheptatetracont-45-en-47-oate (317)

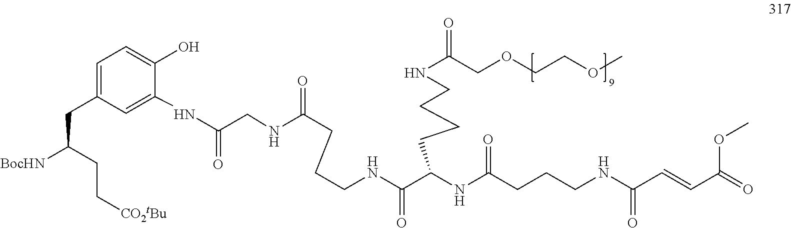

To a solution of compound 316 (213 mg, 0.223 mmol) and compound 69 (144 mg, 0.378 mmol) in DCM (10 mL), was added EDC·HCl (74 mg, 0.386 mmol). The reaction was stirred at r.t. for 3 hours, and then concentrated and purified by preparative HPLC to give compound 317 (171 mg, 59% yield). MS-ESI (m/z): calcd. for C₆₂H₁₀₅N₇O₂₃ [M+H]⁺ 1316.73; found, 1317.66.

### Example 292. Synthesis of (R)-4-amino-5-(4-hydroxy-3-((S)-37-(4-((E)-4-methoxy-4-oxobut-2-enamido)butanamido)-31, 38, 43-trioxo-2, 5, 8, 11, 14, 17, 20, 23, 26, 29-decaoxa-32, 39, 44-triazahexatetracontan-46-amido)phenyl)pentanoic acid(318)

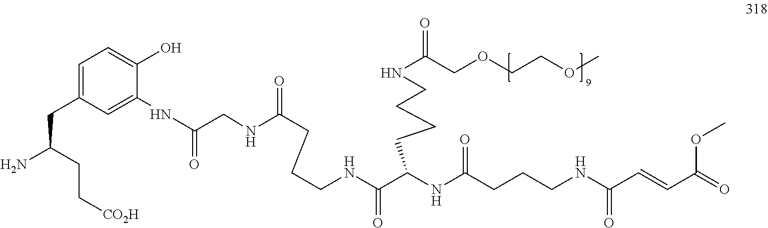

Compound 317 (171 mg, 0.130 mmol) was dissolved in DCM (3 mL) and TFA (1 mL). The solution was stirred at r.t. for 2 hours and concentrated to give compound 318 (0.15 g, 100% yield). MS-ESI (m/z): calcd. for C₅₃H₈₉N₇O₂₁ [M+H]⁺ 1160.61; found, 1161.26.

### Example 293. Synthesis of (R)-4-(2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxamido)-5-(4-hydroxy-3-((S)-37-(4-((E)-4-methoxy-4-oxobut-2-enamido)butanamido)-31, 38, 43-trioxo-2, 5, 8, 11, 14, 17, 20, 23, 26, 29-decaoxa-32, 39, 44-triazahexatetracontan-46-amido)phenyl)pentanoic acid (319)

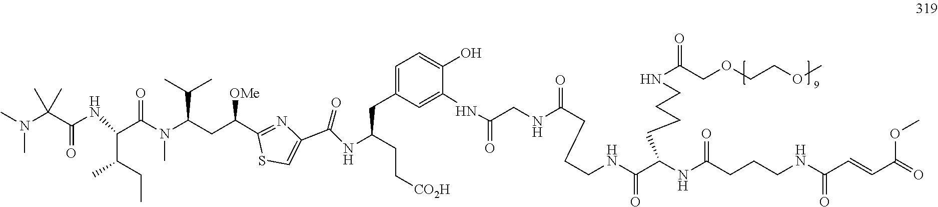

To a solution of compound 318 (150 mg, 0.129 mmol) and Tub-1 (89 mg, 0.129 mmol) in DMF (2 mL) was added N,N-diisopropylethylamine (0.021 mL, 0.129 mmol). The reaction was stirred at r.t. for 1.5 hours, and directly purified by preparative HPLC to give compound 319 (17 mg, 8% yield). MS-ESI (m/z): calcd. for C₇₈H₁₂₉N₁₁O₂₆S [M+H]⁺ 1668.88; found, 1669.34.

### Example 294. Synthesis of tert-butyl (R)-4-((tert-butoxycarbonyl)amino)-5-(3-((S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-amido)-4-hydroxyphenyl)pentanoate (320)

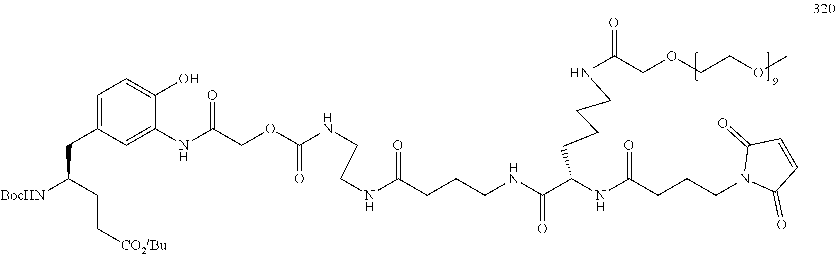

Compound 245 (327 mg, 0.324 mmol) and compound 69 (148 mg, 0.389 mmol) were dissolved in DCM (10 mL), EDC·HCl (75 mg, 0.389 mmol) was added, and the reaction was stirred for 1 h, and then directly taken to the next step. MS-ESI (m/z): calcd. for C₆₄H₁₀₆N₈O₂₄ [M+H]⁺ 1371.73; found, 1372.41.

### Example 295. Synthesis of (R)-4-amino-5-(3-((S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-amido)-4-hydroxyphenyl)pentanoic acid (321)

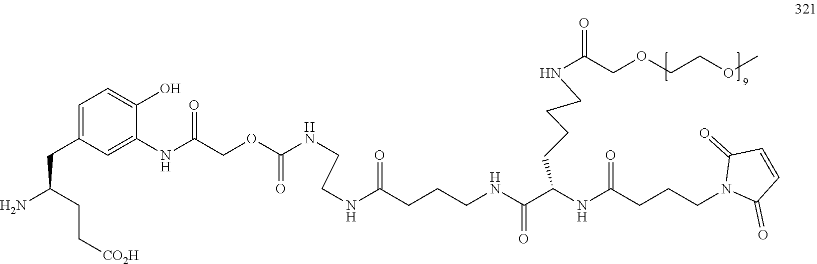

To the reaction mixture of previous step, 10 mL of TFA was added. The reaction was stirred for 1 h, concentrated and purified by preparative HPLC to give compound 321 (0.20 g, 51% yield). MS-ESI (m/z): calcd. for C₅₅H₉₀N₈O₂₂ [M+H]⁺ 1215.62; found, 1216.25.

### Example 296. Synthesis of (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 43, 48-tetraoxo-2, 5, 8, 11, 14, 17, 20, 23, 26, 29, 49-undecaoxa-32, 39, 44, 47-tetraazahenpentacontan-51-amido)-4-hydroxyphenyl)pentanoic acid (322)

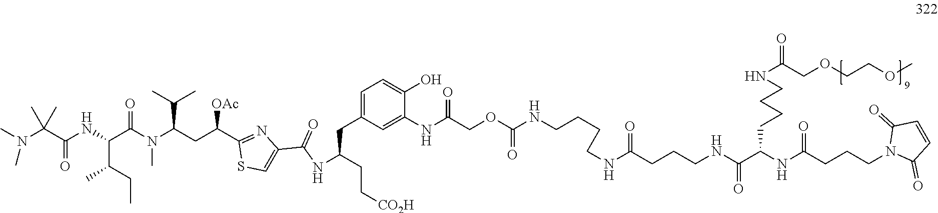

Compound 321 (200 mg, 0.165 mmol) and Tub-1 (114 mg, 0.165 mmol) were dissolved in DMF (2 mL), N,N-diisopropylethylamine (0.027 mL, 0.165 mmol) was added and stirred for 4 hours. The reaction was directly purified by preparative HPLC to give compound 322 (94 mg, 33% yield). MS-ESI (m/z): calcd. for C₈₀H₁₃N₁₂O₂₇S [M+H]⁺ 1723.89; found, 1724.81.

### Example 297. Synthesis of 2, 5-dioxopyrrolidin-1-yl (S)-(37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-oyl)glycinate (323)

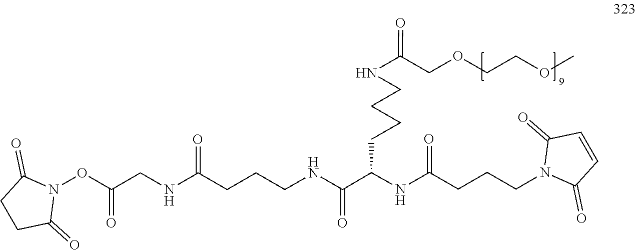

To a solution of compound 234 (1.0 g, 1.08 mmol) in DCM (20 mL) were added NHS (0.15 g, 1.30 mmol) and EDC·HCl (0.43 g, 2.17 mmol). The reaction mixture was stirred at r.t. for 2 hours and quenched with water. The layers were separated and the organic phase was washed with brine, dried over anhydrous sodium sulfate, filtered and concentrated to give compound 323 (1.1 g, 99% yield). MS-ESI (m/z): calcd. for C₄₅H₇₅N₆O₂₀ [M+H]⁺ 1019.50; found, 1019.50.

### Example 298. Synthesis of tert-butyl (R)-4-((tert-butoxycarbonyl)amino)-5-(3-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)-4-hydroxyphenyl)pentanoate (324)

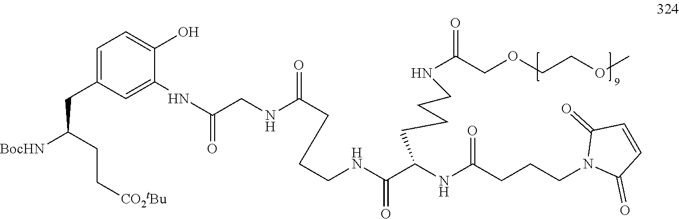

Compound 323 from previous step (1.1 g, 1.08 mmol) was dissolved in THF (10 mL). Compound 69 (215.0 mg, 0.5 mmol) was added to the solution and stirred at r.t. overnight. The reaction mixture was concentrated and purified by a silica gel column (6% MeOH/DCM) to give compound 324 (200 mg, 15% yield). MS-ESI (m/z): calcd. for C₆₁H₁₀₂N₇O₂₂ [M+H]⁺ 1284.70; found, 1284.70.

### Example 299. Synthesis of (R)-4-amino-5-(3-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)-4-hydroxyphenyl)pentanoic acid (325)

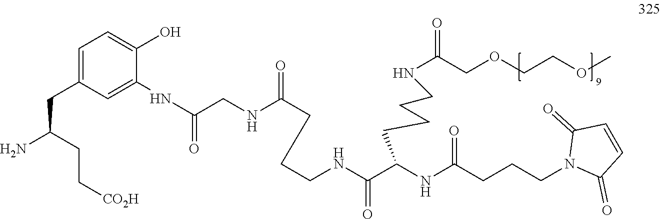

A solution of compound 324 (180 mg, 0.15 mmol) in DCM (4 mL) was treated with TFA (2 mL) at r.t. for 2 hours, concentrated, triturated with MTBE (10 mL) to give compound 325 (174 mg, >100% yield). MS-ESI (m/z): calcd. for C₅₂H₈₆N₇O₂₀ [M+H]⁺ 1128.58; found, 1128.58.

### Example 300. Synthesis of (R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)-4-hydroxyphenyl)pentanoic acid (326)

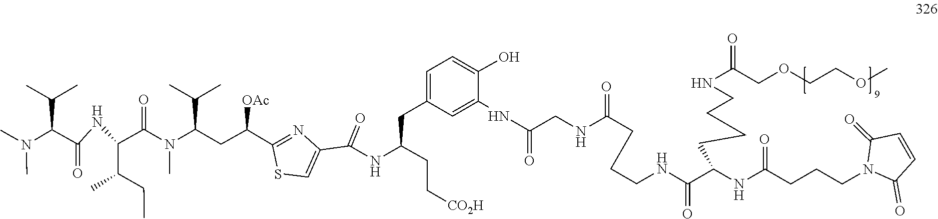

To a solution of compound 325 (174 mg, 0.15 mmol) in DMF (0.5 mL), Tub-4 (98 mg, 0.13 mmol) in DMF (0.5 mL) was added, followed by N,N-diisopropylethylamine (40 mg, 0.29 mmol). The solution was stirred at r.t. for 30 min and then concentrated. The residue was purified by preparative HPLC to give compound 326 (34 mg, 14% yield). MS-ESI (m/z): calcd. for C₇₈H₁₂₈N₁₁O₂₅S [M+H]⁺ 1650.87; found, 1650.87.

### Example 301. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 43-trioxo-2, 5, 8, 11, 14, 17, 20, 23, 26, 29-decaoxa-32, 39, 44-triazahexatetracontan-46-amido)-4-hydroxyphenyl)-2-methylpentanoic acid (328)

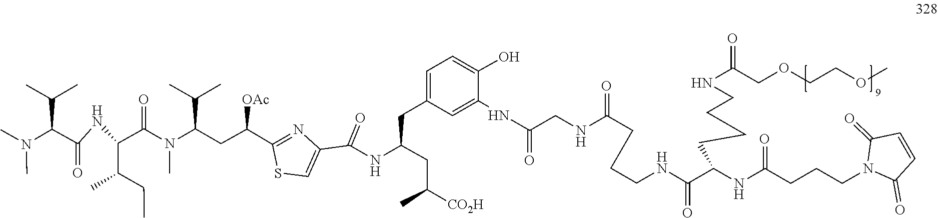

To a solution of Tub-4 (95 mg, 0.13 mmol) and compound 327 (150 mg, 0.13 mmol) in DMF (2 mL) was added N,N-diisopropylethylamine (34 mg, 0.36 mmol) at 0° C. The reaction was warmed to r.t. and stirred for 2 hours, concentrated, then purified by preparative HPLC (water/acetonitrile) to give compound 328 (50 mg, 23% yield). MS-ESI (m/z): calcd. for C₇₉H₁₃₀N₁₁O₂₅S [M+H]⁺ 1666.01; found, 1666.01.

### Example 302. Synthesis of (R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-amido)-4-hydroxyphenyl)pentanoic acid (330)

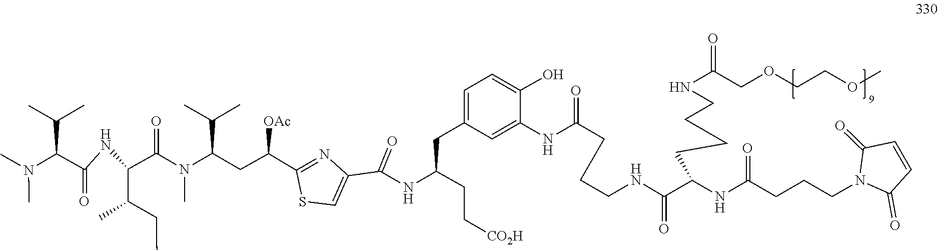

To a solution of Tub-4 (49 mg, 0.070 mmol) and compound 329 (74 mg, 0.070 mmol) in DMF (2 mL) was added N,N-diisopropylethylamine (0.036 mL, 0.28 mmol) at r.t. The reaction was stirred for 3 hours and concentrated, then purified by preparative HPLC (water/acetonitrile) to give compound 330 (51 mg, 46% yield). MS-ESI (m/z): calcd. for C₇₆H₁₂₄N₁₀O₂₄S [M+H]⁺ 1593.85; found, 1594.62.

### Example 303. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-amido)-4-hydroxyphenyl)-2-methylpentanoic acid (332)

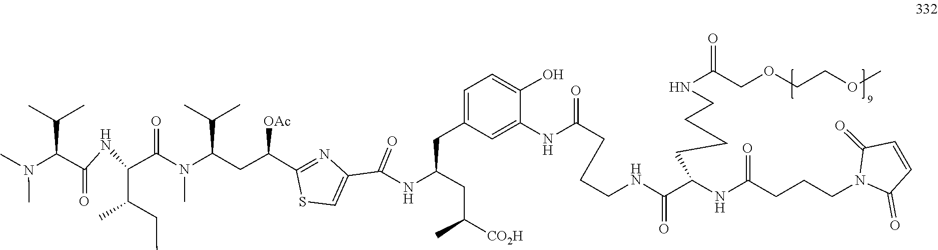

To a solution of Tub-4 (100 mg, 0.14 mmol) and compound 331 (146 mg, 0.14 mmol) in DMF (2 mL) was added N,N-diisopropylethylamine (0.073 mL, 0.42 mmol) at r.t. The reaction was stirred for 3.5 hours and concentrated, then purified by preparative HPLC (water/acetonitrile) to give compound 332 (82 mg, 37% yield). MS-ESI (m/z): calcd. for C₇₅H₁₂₂N₁₀O₂₃S [M+H]⁺ 1563.84; found, 1564.46.

### Example 304. Synthesis of (R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-37-(4-(2, 5-dioxo-2, 5-dihydro-1H-pyrrol-1-yl)butanamido)-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (334)

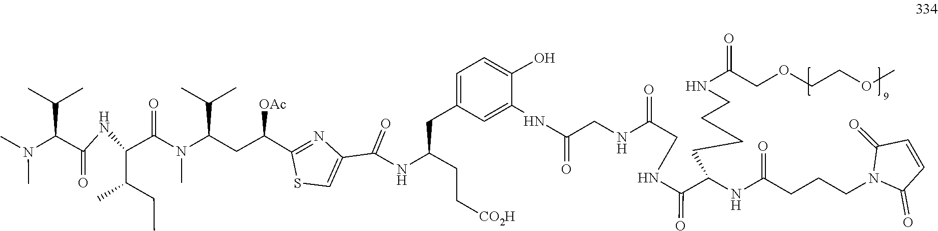

To a solution of Tub-4 (117 mg, 0.17 mmol) and compound 333 (180 mg, 0.17 mmol) in DMF (2 mL) was added N,N-diisopropylethylamine (45 mg, 0.33 mmol) at r.t. The reaction was stirred for 2 hours and concentrated, then purified by preparative HPLC (water/acetonitrile) to give compound 334 (73 mg, 27% yield). MS-ESI (m/z): calcd. for C₇₆H₁₂₄N₁₁O₂₅S [M+H]⁺ 1622.84; found, 1622.84.

### Example 305. Synthesis of (R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (335)

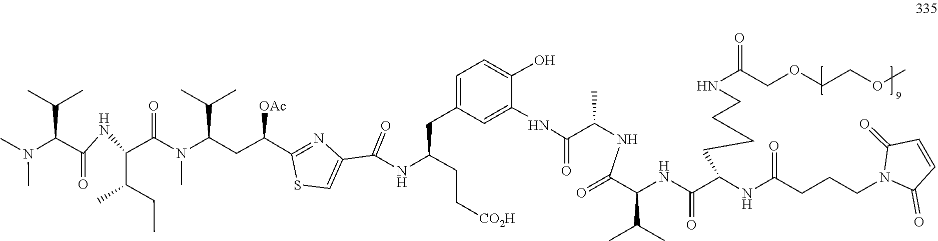

To a solution of Tub-4 (185 mg, 0.256 mmol) and compound 316 (300 mg, 0.256 mmol) in DMF (5 mL) was added N,N-diisopropylethylamine (66 mg, 0.512 mmol) at 0° C. The reaction was stirred at 0° C. for 2 hours and concentrated, then purified by preparative HPLC (water/acetonitrile) to give compound 335 (120 mg, 29% yield). MS-ESI (m/z): calcd. for C₈₀H₁₃₁N₁₁O₂₅S [M+H]⁺ 1678.90; found, 1679.30.

### Example 306. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)-2-methylpentanoic acid (336)

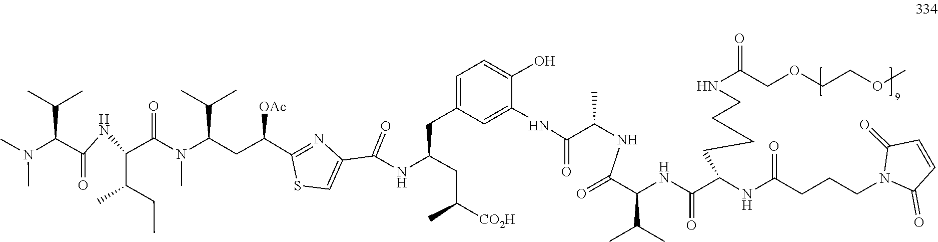

To a solution of Tub-2 (180 mg, 0.256 mmol) and compound 257 (300 mg, 0.256 mmol) in DMF (5 mL) was added N,N-diisopropylethylamine (66 mg, 0.512 mmol) at 0° C. The reaction was stirred at 0° C. for 2 hours and concentrated, then purified by preparative HPLC (water/acetonitrile) to give compound 336 (90 mg, 20% yield). MS-ESI (m/z): calcd. for C₈₀H₁₃₃N₁₁O₂₄S [M+H⁺] 1664.92; found, 1665.50.

### Example 307. Synthesis of (R)-4-(2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxamido)-5-(3-((37S, 40S, 43S)-37-(4-(2, 5-dioxo-2, 5-dihydro-1H-pyrrol-1-yl)butanamido)-40-isopropyl-43-methyl-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid (337)

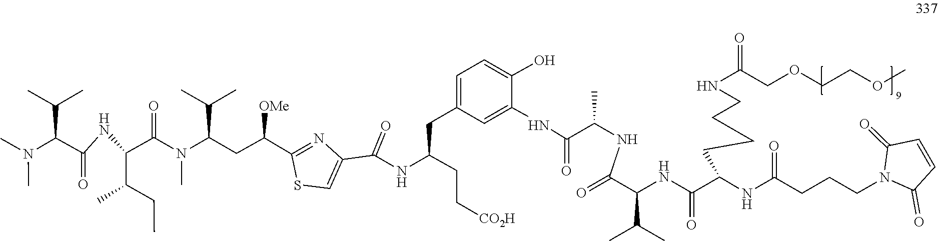

To a solution of Tub-2 (58.1 mg, 0.085 mmol) and compound 316 (128.0 mg, 0.094 mmol) in DMF (2 mL) was added N,N-diisopropylethylamine (20 mg, 0.17 mmol) at 0° C. The reaction was warmed to r.t. and stirred for 2 hours and concentrated, then purified by preparative HPLC (water/acetonitrile) to give compound 337 (26 mg, 19% yield). MS-ESI (m/z): calcd. for C₇₉H₁₃₂N₁₁O₂₄S [M+H]⁺ 1650.91; found, 1650.91.

### Example 308. Synthesis of (R)-4-(2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxamido)-5-(3-((S)-37-(4-(2, 5-dioxo-2, 5-dihydro-1H-pyrrol-1-yl)butanamido)-31, 38, 41-trioxo-2, 5, 8, 11, 14, 17, 20, 23, 26, 29-decaoxa-32, 39, 42-triazatetratetracontan-44-amido)-4-hydroxyphenyl)pentanoic acid(338)

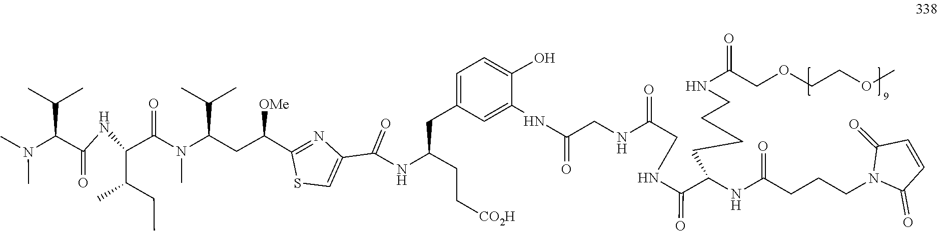

To a solution of Tub-2 (37.0 mg, 0.055 mmol) and compound 333 (60.0 mg, 0.055 mmol) in DMF (2 mL) was added N,N-diisopropylethylamine (0.009 mL, 0.055 mmol) at 0° C. The reaction was warmed to r.t. and stirred for 2.5 hours and concentrated, then purified by preparative HPLC (water/acetonitrile) to give compound 338 (52 mg, 60% yield).b MS-ESI (m/z): calcd. for C₇₅H₁₂₃N₁₁O₂₄S [M+H]⁺ 594.83; found, 1596.26.

### Example 309. Synthesis of (4-(benzyloxy)-5-methoxy-2-nitrophenyl)((2R)-2-(hydroxymethyl)cyclopentyl)methanone (342)

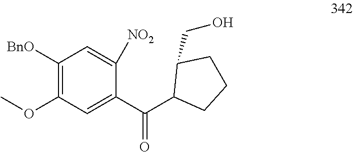 [SMILES: COc1cc(C(=O)C2CCC[C@H]2CO)c([N+](=O)[O-])cc1OCc1ccccc1]

To a solution of compound 340 (30.3 g, 0.1 mol), compound 341 (10.1 g, 0.1 mol) in DMF (500 mL) were added triethylamine (20.1 g, 0.2 mol) and HATU (49.4 g, 0.13 mol) at r.t. The reaction was stirred at r.t. for 2 hours, diluted with DCM (2 L), washed twice with water, brine, dried over anhydrous sodium sulfate, filtered and concentrated to give a crude product, which is purified by a silica gel column (EA/PE=30%-100%) to give the title compound (36.8 g, 95% yield).

### Example 310. Synthesis of (1R)-2-(4-(benzyloxy)-5-methoxy-2-nitrobenzoyl)cyclopentane-1-carbaldehyde (343)

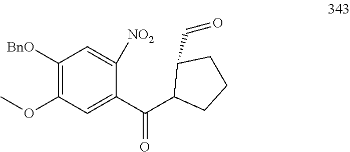 [SMILES: COc1cc(C(=O)C2CCC[C@H]2C=O)c([N+](=O)[O-])cc1OCc1ccccc1]

Under N₂, a solution of oxalyl chloride (4.34 g, 34.19 mmol) in DCM (150 mL) was cooled over dry ice/acetone batch, to which a solution of DMSO (5.57 g, 71.23 mmol) in 30 mL of DCM was added dropwise, and the reaction was kept below −65° C. for 20 min. after the addition. A solution of compound 342 (11.01 g, 28.49 mmol) in 100 mL of DCM was added dropwise, and the reaction was kept below −65° C. and stirred for 20 minutes. A solution of TEA (14.42 g, 142.47 mmol) in 60 mL of DCM was added dropwise at last. After the addition was completed, the dry ice/acetone bath was removed, and the reaction was gradually warmed to r.t. and stirred for 1 hour, then washed with 0.2N HCl, brine, and dried over anhydrous sodium sulfate, filtered and concentrated. Column chromatography purification (EA/PE=20%-100%) gave the title compound (9.1 g, 83% yield).

### Example 311. Synthesis of (S)-8-hydroxy-7-methoxy-1,2,3,10,11,11a-hexahydro-5H-benzo[e]pyrrolo[1,2-a][1,4]diazepin-5-one (344)

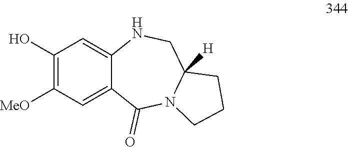 [SMILES: COc1cc2c(cc1O)NC[C@@H]1CCCN1C2=O]

Compound 343 (9.1 g, 23.67 mmol) in methanol (400 mL), palladium/carbon (2.0 g, 10 wt %) were charged into a hydrogenation reaction flask, and the reaction was stirred under hydrogen at r.t. overnight, filtered and concentrated to give the title compound (5.8 g, 98% yield).

### Example 312. Synthesis of(S)-8-((tert-butyldimethylsilyl)oxy)-7-methoxy-1,2,3,10,11,11a-hexahydro-5H-benzo[e]pyrrolo[1,2-a][1,4]diazepin-5-one (345)

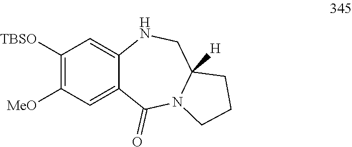 [SMILES: COc1cc2c(cc1O[Si](C)(C)C(C)(C)C)NC[C@@H]1CCCN1C2=O]

To a solution of compound 344 (4.3 g, 17.32 mmol) in DCM (150 mL) were added imidazole (2.4 g, 34.64 mmol) and TBSCl (3.1 g, 20.78 mmol). After the addition, the mixture was reacted at r.t. for 2 hours, washed with brine, dried over anhydrous sodium sulfate, filtered and concentrated. The crude product was purified by column chromatography (EA/PE=20%-100%) to give the title compound (3.7 g, 59% yield).

### Example 313. Synthesis of (9H-fluoren-9-yl)methyl (S)-(2-(8-((tert-butyldimethylsilyl)oxy)-7-methoxy-5-oxo-2,3,11,11a-tetrahydro-1H-benzo[e]pyrrolo[1,2-a][1,4]diazepin-10(5H)-yl)-2-oxoethyl)carbamate (347)

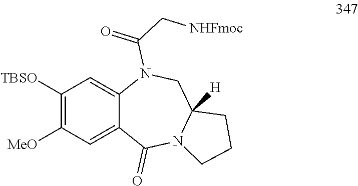

A solution of compound 345 (1.09 g, 3.00 mmol) and pyridine (0.36 g, 4.50 mmol) in DCM (50 mL) was cooled over ice/water. Compound 346 (1.14 g, 3.60 mmol) was added and stirred for 1 hour. The reaction solution was washed with 0.3N HCl solution, brine, dried over anhydrous sodium sulfate, filtered and concentrated. The crude product was purified by column chromatography (EA/PE=20%-100%) to give the title compound (1.35 g, 70% yield).

### Example 314. Synthesis of(S)-10-glycyl-8-hydroxy-7-methoxy-1,2,3,10,11,11a-hexahydro-5H-benzo[e]pyrrolo[1,2-a][1,4]diazepin-5-one (348)

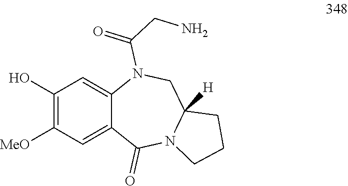 [SMILES: COc1cc2c(cc1O)N(C(=O)CN)C[C@@H]1CCCN1C2=O]

Compound 347 (1.01 g, 1.57 mmol) was dissolved in DCM (20 mL), to which pyrrolidine (1 mL) was added at r.t. After the addition, the reaction was stirred at r.t. for 3 hours and concentrated, purified by column chromatography (MeOH/DCM=0%-30%) to give the title compound (0.41 g, 85% yield).

### Example 315. Synthesis of tert-butyl (S)-(2-(8-hydroxy-7-methoxy-5-oxo-2,3,11,11a-tetrahydro-1H-benzo[e]pyrrolo[1,2-a][1,4]diazepin-10(5H)-yl)-2-oxoethyl)carbamate (349)

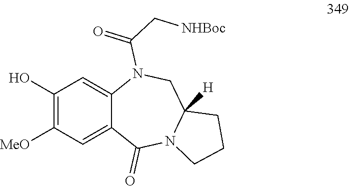 [SMILES: COc1cc2c(cc1O)N(C(=O)CNC(=O)OC(C)(C)C)C[C@@H]1CCCN1C2=O]

Compound 348 (0.41 g, 1.34 mmol) was dissolved in DCM (20 mL) and di-t-butyl dicarbonate (0.35 g, 1.61 mmol) was added at room temperature. After the addition, the reaction was stirred at r.t. for 2 hours and concentrated, purified by column chromatography (EA/PE=20%-100%) to give the title compound (0.20 g, 37% yield).

### Example 316. Synthesis of (4-(benzyloxy)-5-methoxy-2-nitrophenyl)((2R)-2-(((tert-butyldimethylsilyl)oxy)methyl)cyclopentyl)methanone (350)

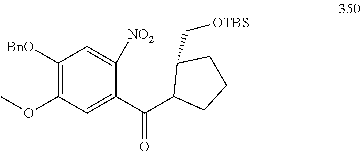 [SMILES: COc1cc(C(=O)C2CCC[C@H]2CO[Si](C)(C)C(C)(C)C)c([N+](=O)[O-])cc1OCc1ccccc1]

To a solution of compound 342 (38.0 g, 0.10 mol) in DCM (1000 mL) imidazole (27.2 g, 0.40 mol) and TBSCl (30.1 g, 0.20 mmol) were added at r.t. under stirring. After the addition, the reaction was stirred at r.t. for 2 hours, and then washed with water, brine, dried over anhydrous sodium sulfate, filtered and concentrated. The crude product was purified by column chromatography (EA/PE=10%-50%) to give the title compound (45.0 g, 90% yield).

### Example 317. Synthesis of (2-amino-4-hydroxy-5-methoxyphenyl)((2R)-2-(((tert-butyldimethylsilyl)oxy)methyl)cyclopentyl)methanone (351)

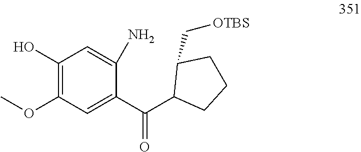 [SMILES: COc1cc(C(=O)C2CCC[C@H]2CO[Si](C)(C)C(C)(C)C)c(N)cc1O]

Compound 350 (45 g, 90 mmol), methanol (800 mL), palladium/carbon (8.0 g, 10 wt %) were charged into a hydrogenation reaction flask. The flask was evacuated and back-filled with hydrogen for three times and stirred at r.t. overnight. Filtration and concentration under oil pump gave the title compound (34 g, 100% yield).

### Example 318. Synthesis of (2-amino-5-methoxy-4-((triisopropylsilyl)oxy)phenyl)((2R)-2-(((tert-butyldimethylsilyl)oxy)methyl)cyclopentyl)methanone (352)

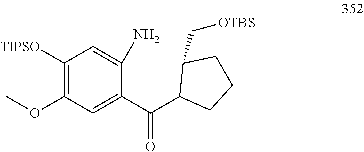

A mixture of compound 351 (19.0 g, 0.05 mol), imidazole (6.8 g, 0.1 mol) and TIPSCl (14.4 g, 0.075 mmol) in ethyl acetate (30 mL) was heated to reflux and stirred for 1 hour. After cooling, DCM was added and the mixture was washed with water, brine, dried over anhydrous sodium sulfate, filtered and concentrated. The crude product was purified by column chromatography (EA/PE=10%-50%) to give the title compound (22 g, 82% yield).

### Example 319. Synthesis of allyl (2-((2R)-2-(((tert-butyldimethylsilyl)oxy)methyl)cyclopentane-1-carbonyl)-4-methoxy-5-((triisopropylsilyl)oxy)phenyl)carbamate (353)

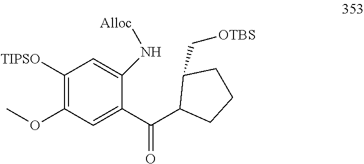

To a solution of compound 352 (92.7 g, 0.173 mol) in DCM (500 mL) was added pyridine (31.3 g, 0.397 mol) at r.t. under stirring. The mixture was cooled over dry ice/acetone bath, and allyl chloroformate (24.97 g, 0.207 mol) was added dropwise at about −65° C. After the addition, dry ice/acetone bath was removed, and the reaction was naturally warmed to room temperature and stirred for 3 hours. The reaction solution was washed with 0.3N HCl solution, brine, dried over anhydrous sodium sulfate, filtered and concentrated to give a crude product (104 g, 99% yield).

### Example 320. Synthesis of allyl (2-((2R)-2-(hydroxymethyl)cyclopentane-1-carbonyl)-4-methoxy-5-((triisopropylsilyl)oxy)phenyl)carbamate (354)

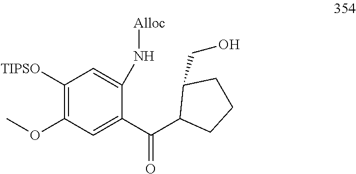

A solution of compound 353 (104 g, 0.173 mol) in acetic acid (600 mL), methanol (85 mL), THF (85 mL) and water (170 mL) was stirred at room temperature for 8 hours, and then diluted with ethyl acetate and washed with water for 3 times, brine once, dried over anhydrous sodium sulfate, filtered and concentrated. The crude product was purified by column chromatography (EA/PE=30%-70%) to give the title compound (59 g, 69% yield over 2 steps).

### Example 321. Synthesis of allyl (11aS)-11-hydroxy-7-methoxy-5-oxo-8-((triisopropylsilyl)oxy)-2,3,11,11a-tetrahydro-1H-benzo[e]pyrrolo[1,2-a][1,4]diazepine-10(5H)-carboxylate (355)

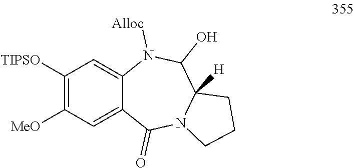

Under N₂, a solution of oxalyl chloride (19.9 g, 155 mmol) in DCM (400 mL) was cooled over dry ice/acetone batch, to which a solution of DMSO (23.4 g, 299 mmol) in 50 mL of DCM was added dropwise, and the reaction was kept below −65° C. for 20 min. after the addition. A solution of compound 354 (59.0 g, 119 mmol) in 200 mL of DCM was added dropwise, and the reaction was kept below −65° C. and stirred for 20 minutes. A solution of TEA (60.6 g, 598 mmol) in 100 mL of DCM was added dropwise at last. After the addition was completed, the dry ice/acetone bath was removed, and the reaction was gradually warmed to r.t. and stirred for 1 hour, then washed with 0.2N HCl, brine, and dried over anhydrous sodium sulfate, filtered and concentrated. Column chromatography purification (EA/PE=10%-50%) gave the title compound (49.7 g, 84% yield).

### Example 322. Synthesis of allyl (11aS)-11-((tert-butyldimethylsilyl)oxy)-7-methoxy-5-oxo-8-((triisopropylsilyl)oxy)-2,3,11,11a-tetrahydro-1H-benzo[e]pyrrolo[1,2-a][1,4]diazepine-10(5H)-carboxylate (356)

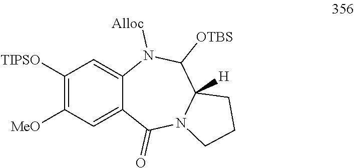

To a solution of compound 355 (5.8 g, 11.82 mmol) in DCM (100 mL) were added 2,6-lutidine (5.07 g, 47.28 mmol) and TBSOTf (9.37 g, 35.46 mmol). After the addition, the reaction was stirred at r.t. for 2 hours, washed with water, brine, dried over anhydrous sodium sulfate, filtered and concentrated. The crude product was purified by column chromatography (EA/PE=10%-50%) to give the title compound (6.3 g, 88% yield).

### Example 323. Synthesis of allyl (11aS)-11-((tert-butyldimethylsilyl)oxy)-8-hydroxy-7-methoxy-5-oxo-2,3,11,11a-tetrahydro-1H-benzo[e]pyrrolo[1,2-a][1,4]diazepine-10(5H)-carboxylate (357)

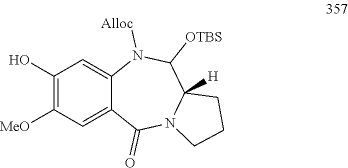

A mixture of compound 356 (6.3 g, 10.41 mmol) and lithium acetate (0.69 g, 10.41 mmol) in DMF (78 mL) and water (1.5 mL) was stirred at r.t. for 4 hours. The reaction solution was diluted with ethyl acetate, washed with water for 3 times, brine once, dried over anhydrous sodium sulfate, filtered and concentrated. The crude product was purified by column chromatography (EA/PE=30%-100%) to give the title compound (3.3 g, 70% yield).

### Example 324. Synthesis of allyl (11aS)-11-((tert-butyldimethylsilyl)oxy)-8-((5-iodopentyl)oxy)-7-methoxy-5-oxo-2,3,11,11a-tetrahydro-1H-benzo[e]pyrrolo[1,2-a][1,4]diazepine-10(5H)-carboxylate (358)

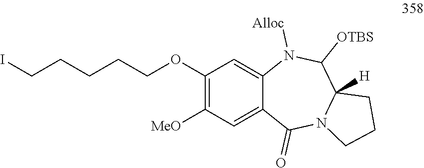

To a solution of compound 357 (1.39 g, 3 mmol) in acetone (100 mL) were added diiodopentane (4.86 g, 15 mmol) and potassium carbonate (0.62 g, 4.5 mmol) with stirring. After the addition, the reaction was heated under reflux for 8 hours, and after cooling, it was directly purified by column chromatography (EA/PE=20%-80%) to give the title compound (1.85 g, 93% yield).

### Example 325. Synthesis of allyl (11aS)-8-((5-(((S)-10-((tert-butoxycarbonyl)glycyl)-7-methoxy-5-oxo-2,3, 5,10,11,11a-hexahydro-1H-benzo[e]pyrrolo[1,2-a][1,4]diazepin-8-yl)oxy)pentyl)oxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate (359)

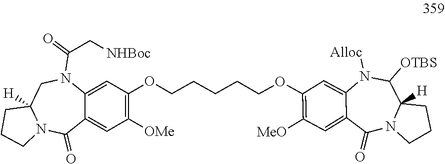

Compound 349 (121 mg, 0.3 mmol) was dissolved in acetone (20 mL), to which compound 359 (197 mg, 0.3 mmol) and potassium carbonate (83 mg, 0.6 mmol) were added with stirring. The reaction was heated under reflux for 8 hours, and after cooling, it was directly purified by column chromatography (MeOH/DCM=0%-10%) to give the title compound (240 mg, 85% yield).

### Example 326. Synthesis of allyl (11aS)-8-((5-(((S)-10-glycyl-7-methoxy-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-5-oxo-2,3,11,11a-tetrahydro-1H-benzo[e]pyrrolo[1,2-a][1,4]diazepine-10(5H)-carboxylate (360)

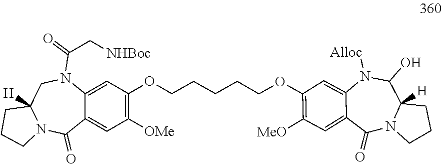

Compound 359 (199.9 mg, 0.21 mmol) was dissolved in 2 mL of TFA and 6 mL of DCM, stirred at r.t. for ˜3 h, diluted with 10 mL of DCM, washed with 5 mL of brine and 5 mL of saturated sodium bicarbonate, dried over anhydrous sodium sulfate, filtered and concentrated to give a pale yellow foamy solid (177.5 mg, 100% yield). MS-ESI (m/z): calcd. for C₃₇H₄₈N₅O₁₀ [M+H]⁺ 721.33; found 721.33.

### Example 327. Synthesis of N—((S)-5-(4-(2,5-dioxo-2,5-dihydro-1H-pyrrol-1-yl)butanamido)-6-(((S)-1-(((S)-1-((2-((S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepin-10(5H)-yl)-2-oxoethyl)amino)-1-oxopropan-2-yl)amino)-1-oxopropan-2-yl)amino)-6-oxohexyl)-2,5,8,11,14,17,20,23,26,29-decaoxahentriacontan-31-amide (361)

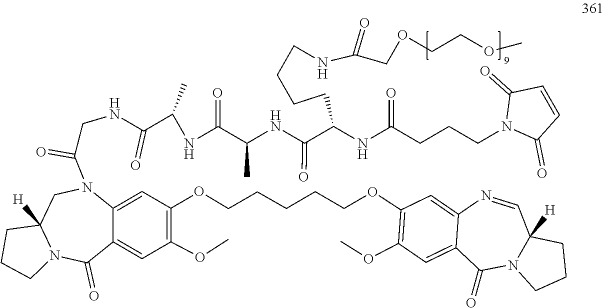

Compound 360 (77.0 mg, 0.11 mmol) was dissolved in 2 mL of DCM, to which pyrrolidine (7.6 mg, 0.11 mmol) and catalytic amount of Pd(PPh₃)₄ were added, and then stirred at r.t. for 20 min. The reaction was cooled to 0-5° C., compound 291 (147.6 mg, 0.16 mmol) in 2 mL of DCM and EDC·HCl (40.9 mg, 0.21 mmol) were added. After stirring for 2 h, the reaction was concentrated, purified by preparative HPLC to give a pale yellow solid (65 mg, 41% yield). MS-ESI (m/z): calcd. for C₇₄H₁₁₁N₁₀O₂₄ [M+H]⁺ 1523.77; found 1523.77.

### Example 328. Synthesis of N-((5S,8S,11S,14S)-14-(4-(2,5-dioxo-2,5-dihydro-1H-pyrrol-1-yl)butanamido)-1-((S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepin-10(5H)-yl)-5,8,11-trimethyl-1,4,7,10,13-pentaoxo-3,6,9,12-tetraazaoctadecan-18-yl)-2,5,8,11,14,17,20,23,26,29-decaoxahentriacontan-31-amide (362)

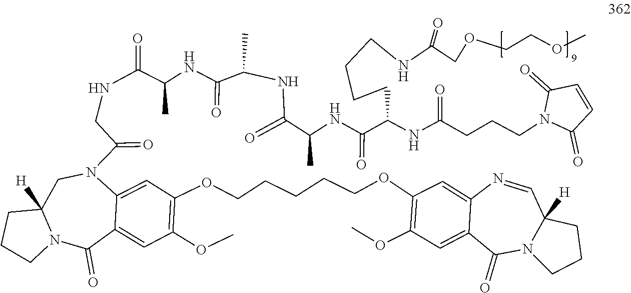

Compound 360 (77.0 mg, 0.11 mmol) was dissolved in 4 mL of DCM, to which pyrrolidine (7.6 mg, 0.11 mmol) and catalytic amount of Pd(PPh₃)₄ were added, and then stirred at r.t. for 20 min. The reaction was cooled to 0-5° C., compound 217 (175.3 mg, 0.17 mmol) in 2 mL of DCM and EDC·HCl (41.0 mg, 0.21 mmol) were added. After stirring for 2 h, the reaction was concentrated, purified by preparative HPLC to give a pale yellow solid (18.6 mg, 11% yield). MS-ESI (m/z): calcd. for C₇₇H₁₁₆N₁₁O₂₅ [M+H]⁺ 1594.81; found 1594.81.

### Example 329. Synthesis of (9H-fluoren-9-yl)methyl (S)-(1-chloro-1-oxopropan-2-yl)carbamate (364)

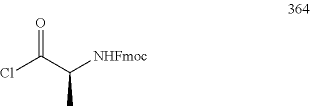

Fmoc-Ala-OH (10.4 g, 33.40 mmol) was dissolved in 100 mL of DCM, to which 6 drops of DMF, 12 mL of SOCl₂ were added. The reaction mixture was heated to 40-50° C., refluxed for 1 h, cooled to r.t. and concentrated. The residue was triturated with 50 mL of n-hexane, filtered and dried to give a white solid (9.7 g, 88% yield). MS-ESI (m/z): calcd. for C₁₈H₁₈NO₄ [M+H]⁺ 312.12; found 312.12.

### Example 330. Synthesis of (9H-fluoren-9-yl)methyl ((S)-1-((S)-8-(benzyloxy)-7-methoxy-5-oxo-2,3,11,11a-tetrahydro-1H-benzo[e]pyrrolo[1,2-a][1,4]diazepin-10(5H)-yl)-1-oxopropan-2-yl)carbamate (366)

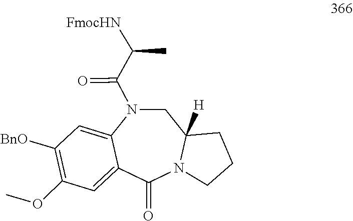

Compound 364 (1.23 g, 3.03 mmol) and compound 365 (1.00 g, 3.03 mmol) were dissolved in 10 mL of DCM, and stirred at r.t. for -5 h. The reaction was diluted with 40 mL of DCM, washed with 40 mL of 0.3N HCl, 50 mL of brine, dried over sodium sulfate, filtered, concentrated to dryness. The residue was purified by a silica gel column (ethyl acetate/petroleum ether) to give a pale yellow solid (1.4 g, 73% yield). MS-ESI (m/z): calcd. for C₃₈H₃₈N₃O₆ [M+H]⁺ 632.27; found 632.27.

### Example 331. Synthesis of(S)-10-(L-alanyl)-8-(benzyloxy)-7-methoxy-1,2,3,10,11,11a-hexahydro-5H-benzo[e]pyrrolo[1,2-a][1,4]diazepin-5-one (367)

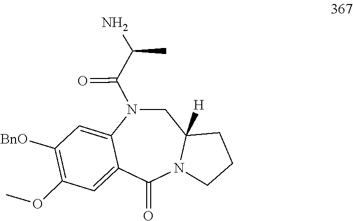 [SMILES: COc1cc2c(cc1OCc1ccccc1)N(C(=O)[C@H](C)N)C[C@@H]1CCCN1C2=O]

A mixture of compound 366 (0.66 g, 1.04 mmol) in 10 mL of DCM, and 1 mL of pyrrolidine was stirred at r.t. for 1 h, and concentrated to give a pale yellow solid (1.06 g, 100% yield). MS-ESI (m/z): calcd. for C₂₃H₂₈N₃O₄ [M+H]⁺ 410.20; found 410.20.

### Example 332. Synthesis of tert-butyl ((S)-1-((S)-8-(benzyloxy)-7-methoxy-5-oxo-2,3,11,11a-tetrahydro-1H-benzo[e]pyrrolo[1,2-a][1,4]diazepin-10(5H)-yl)-1-oxopropan-2-yl)carbamate (368)

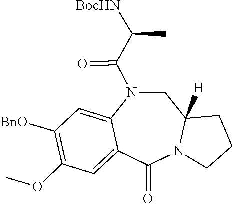 [SMILES: COc1cc2c(cc1OCc1ccccc1)N(C(=O)[C@H](C)NC(=O)OC(C)(C)C)C[C@@H]1CCCN1C2=O]

To a solution of compound 367 (430 mg, 1.05 mmol) in 10 mL of DCM, di-tert-butyl dicarbonate (901.1 mg, 4.13 mmol), DMAP (24 mg, 0.196 mmol) were added, and the mixture was stirred at r.t. for 3 hours and directly purified by a silica gel column (ethyl acetate/petroleum ether) to give a colorless oil (297.1 mg, 55% yield). MS-ESI (m/z): calcd. for C₂₈H₃₆N₃O₆ [M+H]⁺ 510.25; found 510.25.

### Example 333. Synthesis of tert-butyl ((S)-1-((S)-8-hydroxy-7-methoxy-5-oxo-2,3,11,11a-tetrahydro-1H-benzo[e]pyrrolo[1,2-a][1,4]diazepin-10(5H)-yl)-1-oxopropan-2-yl)carbamate (369)

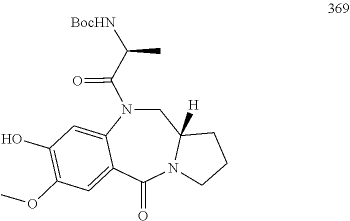 [SMILES: COc1cc2c(cc1O)N(C(=O)[C@H](C)NC(=O)OC(C)(C)C)C[C@@H]1CCCN1C2=O]

Compound 368 (567 mg, 1.11 mmol), methanol (60 mL), palladium/carbon (126 mg, 10 wt %) were charged into a hydrogenation reaction flask. The flask was evacuated and back-filled with hydrogen for three times and stirred at r.t. overnight. Filtration and concentration under oil pump gave a colorless oil (469 mg, 100% yield). MS-ESI (m/z): calcd. for C₂₁H₃₀N₃O₆ [M+H]⁺ 420.21; found 420.21.

### Example 334. Synthesis of allyl (11aS)-8-((5-(((S)-10-((tert-butoxycarbonyl)-L-alanyl)-7-methoxy-5-oxo-2,3,5,10,11,11a-hexahydro-1H-benzo[e]pyrrolo[1,2-a][1,4]diazepin-8-yl)oxy)pentyl)oxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate (370)

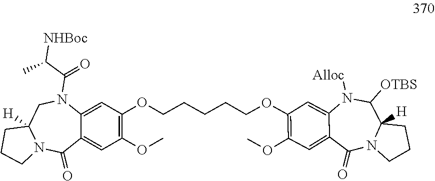

A mixture of compound 369 (254 mg, 0.61 mmol) and compound 358 (598 mg, 0.91 mmol) in 40 mL of acetone, and potassium carbonate (167.0 mg, 1.21 mmol) was heated to reflux and stirred for 7 hours. The reaction was concentrated and purified by a silica gel column (ethyl acetate/petroleum ether and then dichloromethane/methanol) to give a light yellow solid (413 mg, 72% yield). MS-ESI (m/z): calcd. for C₄₉H₇₂IN₅O₁₂Si [M+H]⁺ 950.49; found 950.49.

### Example 335. Synthesis of allyl (11aS)-8-((5-(((S)-10-(L-alanyl)-7-methoxy-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-5-oxo-2,3,11,11a-tetrahydro-1H-benzo[e]pyrrolo[1,2-a][1,4]diazepine-10(5H)-carboxylate (371)

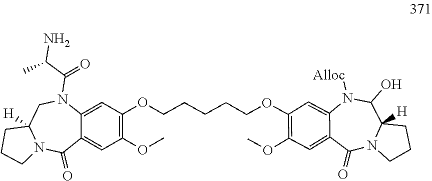

Compound 370 (413 mg, 0.43 mmol) was dissolved in 2 mL of TFA and 6 mL of DCM, stirred at r.t. for 2 h, and concentrated. The residue was diluted with 20 mL of DCM, washed with 6 mL of brine, 6 mL of saturated sodium bicarbonate, dried over anhydrous sodium sulfate, filtered and concentrated to give a light yellow solid (334 mg, 100% yield). MS-ESI (m/z): calcd. for C₃₈H₅₀IN₅O₁₀ [M+H]⁺ 736.35; found 736.35.

### Example 336. Synthesis of N—((S)-5-(4-(2,5-dioxo-2,5-dihydro-1H-pyrrol-1-yl)butanamido)-6-((4-(((S)-1-((S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepin-10(5H)-yl)-1-oxopropan-2-yl)amino)-4-oxobutyl)amino)-6-oxohexyl)-2,5,8,11,14,17,20,23,26,29-decaoxahentriacontan-31-amide (372)

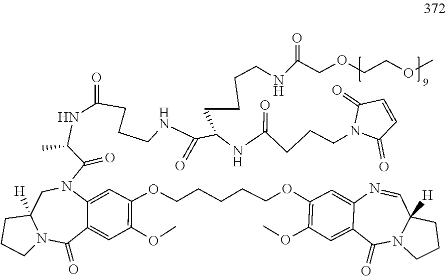

Compound 371 (79.9 mg, 0.11 mmol) was dissolved in 8 mL of DCM, to which pyrrolidine (7.7 mg, 0.11 mmol) and catalytic amount of Pd(PPh₃)₄ were added, and then stirred at r.t. for 30 min. The reaction was cooled to 0-5° C., compound 242 (140 mg, 0.16 mmol) in 8 mL of DCM and EDC·HCl (42.0 mg, 0.22 mmol) were added. After stirring for 2 h, the reaction was concentrated, purified by preparative HPLC to give a brown oil (33 mg, 20% yield). MS-ESI (m/z): calcd. for C₇₃H₁₁₀IN₉O₂₃ [M+H]⁺ 1480.76; found 1480.76.

### Example 337. Synthesis of N—((S)-5-(4-(2,5-dioxo-2,5-dihydro-1H-pyrrol-1-yl)butanamido)-6-((4-((2-(((R)-1-((S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepin-10(5H)-yl)-1-oxopropan-2-yl)amino)-2-oxoethyl)amino)-4-oxobutyl)amino)-6-oxohexyl)-2,5,8,11,14,17,20,23,26,29-decaoxahentriacontan-31-amide (373)

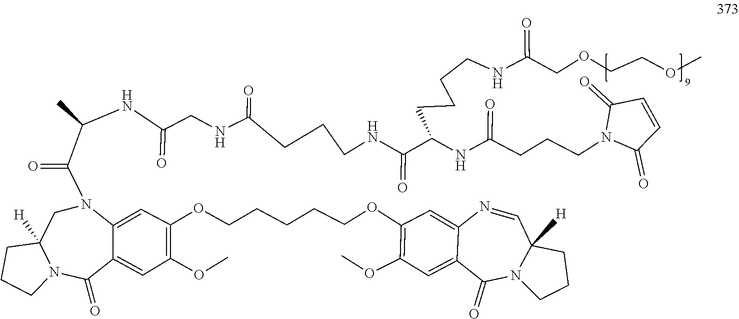

Compound 371 (79.9 mg, 0.11 mmol) was dissolved in 8 mL of DCM, to which pyrrolidine (7.7 mg, 0.11 mmol) and catalytic amount of Pd(PPh₃)₄ were added, and then stirred at r.t. for 30 min. The reaction was cooled to 0-5° C., compound 234 (150 mg, 0.16 mmol) in 8 mL of DCM and EDC·HCl (42.0 mg, 0.22 mmol) were added. After stirring for 2 h, the reaction was concentrated, purified by preparative HPLC to give a light yellow solid (32 mg, 19% yield). MS-ESI (m/z): calcd. for C₇₅H₁₁₃IN₁₀O₂₄ [M+H]⁺ 1537.79; found 1537.79.

### Example 338. Synthesis of N—((S)-5-(4-(2,5-dioxo-2,5-dihydro-1H-pyrrol-1-yl)butanamido)-6-(((S)-1-(((S)-1-(((R)-1-((S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepin-10(5H)-yl)-1-oxopropan-2-yl)amino)-1-oxopropan-2-yl)amino)-1-oxopropan-2-yl)amino)-6-oxohexyl)-2,5,8,11,14,17,20,23,26,29-decaoxahentriacontan-31-amide (374)

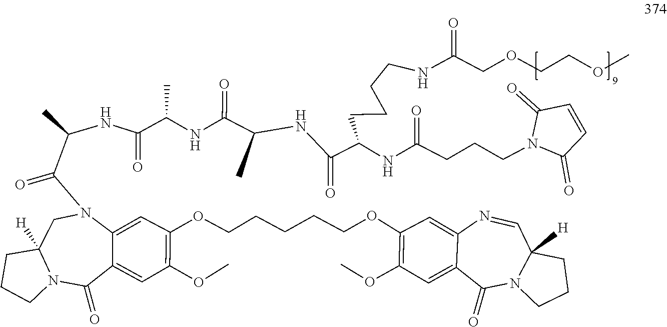

Compound 371 (79.9 mg, 0.11 mmol) was dissolved in 8 mL of DCM, to which pyrrolidine (7.7 mg, 0.11 mmol) and catalytic amount of Pd(PPh₃)₄ were added, and then stirred at r.t. for 30 min. The reaction was cooled to 0-5° C., compound 291 (164 mg, 0.17 mmol) in 10 mL of DCM and EDC·HCl (42.0 mg, 0.22 mmol) were added. After stirring for 2 h, the reaction was concentrated, purified by preparative HPLC to give a brown oil (56 mg, 32% yield). MS-ESI (m/z): calcd. for C₇₅H₁₁₃IN₁₀O₂₄ [M+H]⁺ 1537.79; found 1537.79.

### Example 339. Synthesis of N-((2S,5S,8S,11S,14S)-14-(4-(2,5-dioxo-2,5-dihydro-1H-pyrrol-1-yl)butanamido)-1-((S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepin-10(5H)-yl)-2,5,8,11-tetramethyl-1,4,7,10,13-pentaoxo-3,6,9,12-tetraazaoctadecan-18-yl)-2,5,8,11,14,17,20,23,26,29-decaoxahentriacontan-31-amide (375)

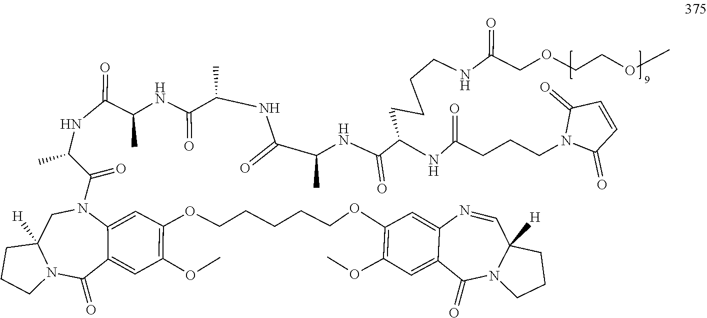

Compound 371 (79.9 mg, 0.11 mmol) was dissolved in 10 mL of DCM, to which pyrrolidine (7.7 mg, 0.11 mmol) and catalytic amount of Pd(PPh₃)₄ were added, and then stirred at r.t. for 30 min. The reaction was cooled to 0-5° C., compound 217 (162 mg, 0.16 mmol) in 10 mL of DCM and EDC·HCl (43.0 mg, 0.22 mmol) were added. After stirring for 2 h, the reaction was concentrated, purified by preparative HPLC to give a brown oil (45 mg, 25% yield). MS-ESI (m/z): calcd. for C₇₈H₁₁₈IN₁₁O₂₅ [M+H]⁺ 1608.82; found 1608.82.

### Example 340. Synthesis of 4-nitrophenyl (S)-8-(benzyloxy)-7-methoxy-5-oxo-2,3,11,11a-tetrahydro-1H-benzo[e]pyrrolo[1,2-a][1,4]diazepine-10(5H)-carboxylate (377)

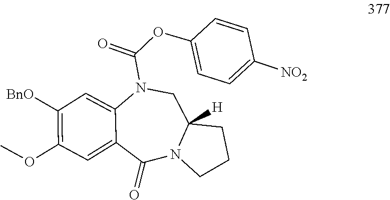 [SMILES: COc1cc2c(cc1OCc1ccccc1)N(C(=O)Oc1ccc([N+](=O)[O-])cc1)C[C@@H]1CCCN1C2=O]

To a solution of compound 365 (2.03 g, 6.0 mmol) in DCM (100 mL) were added DIPEA (0.93 g, 7.2 mmol) and 4-nitrophenyl chloroformate (1.33 g, 6.6 mmol) under stirring. After the addition, the mixture was stirred at r.t. overnight, washed with water, brine, dried over anhydrous sodium sulfate, filtered and concentrated. The crude product was purified by column chromatography (EA/PE=20%-100%) to give the title compound (2.6 g, 86% yield).

### Example 341. Synthesis of 4-((S)-2-((tert-butoxycarbonyl)amino)propanamido)benzyl (S)-8-(benzyloxy)-7-methoxy-5-oxo-2,3,11,11a-tetrahydro-1H-benzo[e]pyrrolo[1,2-a][1,4]diazepine-10(5H)-carboxylate (379)

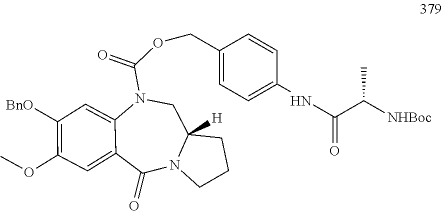 [SMILES: COc1cc2c(cc1OCc1ccccc1)N(C(=O)OCc1ccc(NC(=O)[C@H](C)NC(=O)OC(C)(C)C)cc1)C[C@@H]1CCCN1C2=O]

A solution of compound 378 (0.71 g, 2.4 mmol) in anhydrous THF (5 mL) and DMA (10 mL) was cooled to below 0° C. in an ice-salt batch. LiHMDS (2.4 mL, 1 mol/L) was added dropwise under N₂, and the reaction was kept below 0° C. for 20 minutes. A solution of tert-butyl (S)-(1-((4-(hydroxymethyl)-phenyl)amino)-1-oxopropan-2-yl)carbamate (compound 377) (1.01 g, 2 mmol) in 10 mL of THF was added dropwise, and the reaction was kept below 0° C. for 20 minutes, and warmed to r.t. and stirred for 4 hours. The reaction solution was diluted with DCM, washed with ammonium chloride solution, brine, and dried over anhydrous sodium sulfate, filtered and concentrated. The crude product was purified by column chromatography (EA/PE=20%-100%) to give the title compound (0.84 g, 63% yield).

### Example 342. Synthesis of 4-((S)-2-((tert-butoxycarbonyl)amino)propanamido)benzyl (S)-8-hydroxy-7-methoxy-5-oxo-2,3,11,11a-tetrahydro-1H-benzo[e]pyrrolo[1,2-a][1,4]diazepine-10(5H)-carboxylate (380)

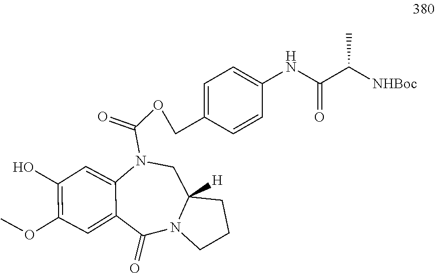 [SMILES: COc1cc2c(cc1O)N(C(=O)OCc1ccc(NC(=O)[C@H](C)NC(=O)OC(C)(C)C)cc1)C[C@@H]1CCCN1C2=O]

Compound 379 (1.17 g, 1.77 mmol), methanol (25 mL), palladium/carbon (0.20 g, 10 wt %) were charged into a hydrogenation reaction flask. The flask was evacuated and back-filled with hydrogen for three times and stirred at 0° C. for 1 h. Filtration and concentration under oil pump gave the title compound (0.91 g, 90% yield).

### Example 343. Synthesis of allyl (11aR)-8-((5-(((R)-10-(((4-((S)-2-((tert-butoxycarbonyl)amino)propanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)pentyl)oxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate (381)

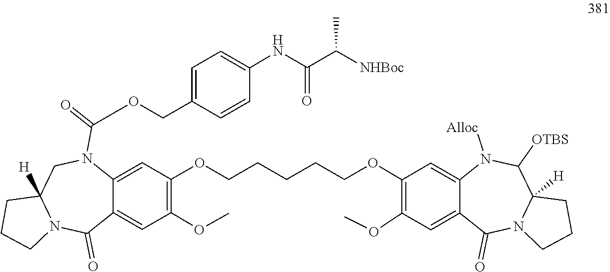

A mixture of compound 380 (0.91 g, 1.6 mmol) and compound 358 (1.37 g, 2.1 mmol) in 80 mL of acetone, and potassium carbonate (0.44 g, 3.2 mmol) was heated to reflux and stirred for 8 hours. The reaction was directly purified by a silica gel column (0-10% MeOH/DCM) to give the title compound (1.4 g, 79% yield).

### Example 344. Synthesis of allyl (11aR)-8-((5-(((R)-10-(((4-((S)-2-aminopropanamido)benzyl)oxy)carbonyl)-7-methoxy-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-5-oxo-2,3,11,11a-tetrahydro-1H-benzo[e]pyrrolo[1,2-a][1,4]diazepine-10(5H)-carboxylate (382)

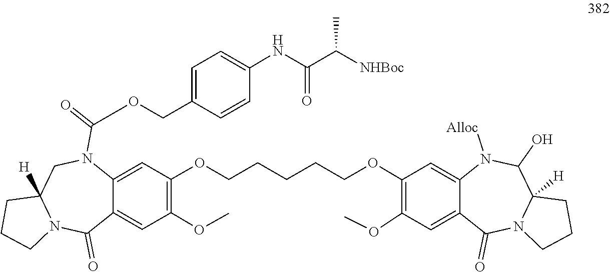

Compound 381 (0.70 g, 0.64 mmol) was dissolved in 15 mL of DCM and cooled to 5° C., to which TFA (5 mL) was added and stirred at r.t. for 40 min. The reaction was diluted with DCM, washed with 10% saturated sodium bicarbonate and brine, dried over anhydrous sodium sulfate, filtered and concentrated. The crude product was purified by a silica gel column (0-15% MeOH/DCM) to give the title compound (0.38 g, 66% yield).

### Example 345. Synthesis of 4-((S)-2-aminopropanamido)benzyl (R)-7-methoxy-8-((5-(((R)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepine-10(5H)-carboxylate (383)

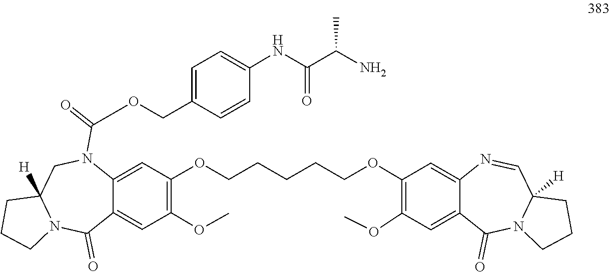 [SMILES: COc1cc2c(cc1OCCCCCOc1cc3c(cc1OC)C(=O)N1CCC[C@@H]1CN3C(=O)OCc1ccc(NC(=O)[C@H](C)N)cc1)N=C[C@H]1CCCN1C2=O]

Compound 382 (62 mg, 0.07 mmol) was dissolved in 2 mL of DCM, to which pyrrolidine (4.7 mg, 0.07 mmol) and catalytic amount of Pd(PPh₃)₄ (1 mg) were added, and then stirred at r.t. for 30 min. The reaction was diluted with DMF and evaporated to remove DCM. The resulting crude product in DMF was used directly in the next step. MS-ESI (m/z): calcd. for C₄₂H₅₀N₆O₉ [M+H]⁺: 783.90; found 783.85.

### Example 346. Synthesis of 4-((37S,40S,43S,46S,49S)-37-(4-(2,5-dioxo-2,5-dihydro-1H-pyrrol-1-yl)butanamido)-40,43,46,49-tetramethyl-31,38,41,44,47-pentaoxo-2,5,8,11,14,17,20,23,26,29-decaoxa-32,39,42,45,48-pentaazapentacontan-50-amido)benzyl (S)-7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepine-10(5H)-carboxylate (384)

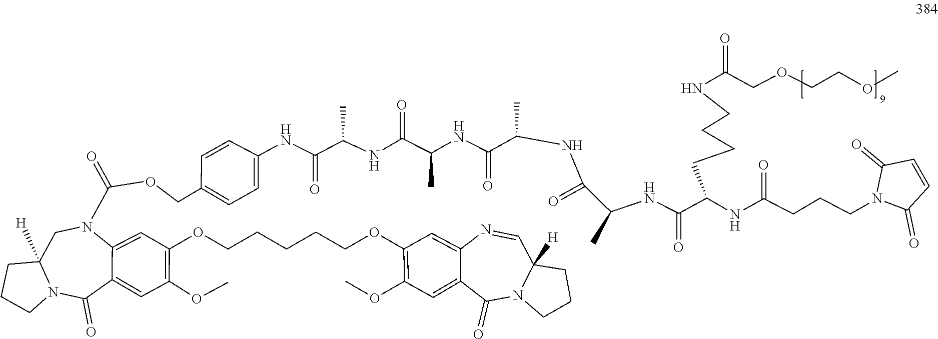

Compound 382 (100 mg, 0.11 mmol) was dissolved in 10 mL of DCM, to which pyrrolidine (8.0 mg, 0.11 mmol) and catalytic amount of Pd(PPh₃)₄ were added, and then stirred at r.t. for 30 min. The reaction was cooled to 0-5° C., compound 217 (156 mg, 0.17 mmol) in 10 mL of DCM and EDC·HCl (86.6 mg, 0.45 mmol) were added. After stirring for 2 h, the reaction was concentrated, purified by preparative HPLC to give a white solid (43 mg, 22% yield). MS-ESI (m/z): calcd. for C₈₆H₁₂₅IN₁₂O₂₇ [M+H]⁺ 1756.87; found 1756.87.

### Example 347. Synthesis of ((((2S,5S,8S,11S,14S,22S,23S,31S,34S,37S,40S,43S)-22,23-bis(2,5-dioxo-2,5-dihydro-1H-pyrrol-1-yl)-2,5,8,11,34,37,40,43-octamethyl-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioyl)bis(azanediyl))bis(4,1-phenylene))bis(methylene) (11aS,11a′S)-bis(7-methoxy-8-((5-(((S)-7-methoxy-5-oxo-2,3,5,11a-tetrahydro-1H-benzo[e]pyrrolo[1,2-a][1,4]diazepin-8-yl)oxy)pentyl)oxy)-5-oxo-2,3,11,11a-tetrahydro-1H-benzo[e]pyrrolo[1,2-a][1,4]diazepine-10(5H)-carboxylate) (385)

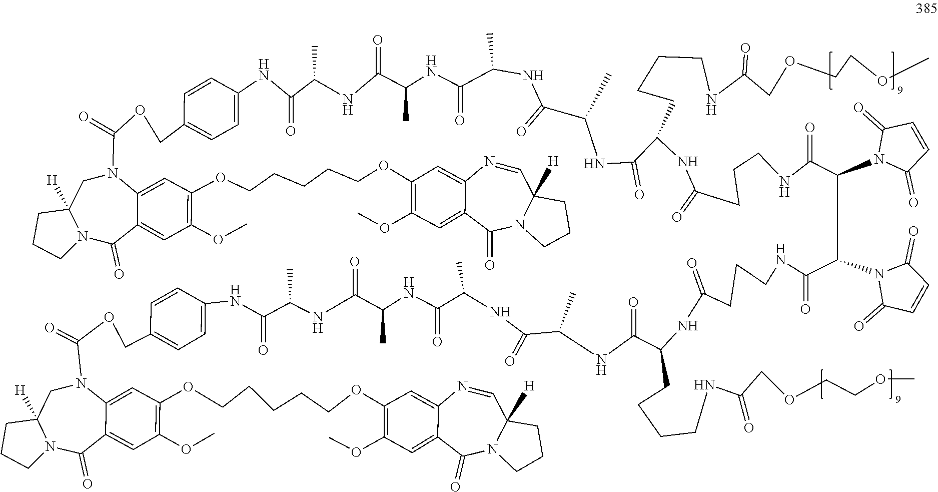

A solution of compound 136 (73 mg, 0.035 mmol), HATU (40 mg, 0.105 mmol) in DMF (1 mL) was stirred at r.t. for 15 min. and then the crude product of 383 (0.07 mmol) in DMF was added to the reaction solution, followed by DIPEA (14 mg, 0.105 mmol). After stirring at r.t. for 30 min., the reaction mixture was directly purified by preparative HPLC to give a pale yellow solid (45 mg, 18% yield). MS-ESI (m/z): calcd. for C₁₇₆H₂₅₂N₂₆O₅₆ [2M+H]⁺:1815.08; found 1815.11.

### Example 348. Synthesis of(S)-2,5-dioxopyrrolidin-1-yl 37-(4-(2,5-dioxo-2,5-dihydro-1H-pyrrol-1-yl)butanamido)-31,38,43-trioxo-2,5,8,11,14,17,20,23,26,29-decaoxa-32,39,44-triazahexatetracontan-46-oate (387)

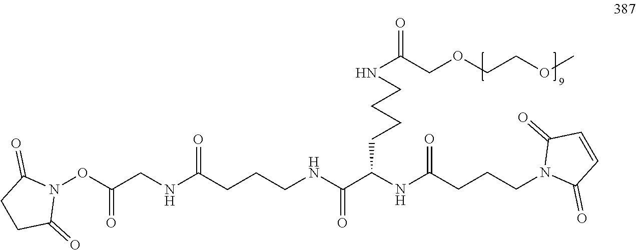

Compound 234 (12.6 g, 13.7 mmol) was dissolved in DCM (150 mL), N-hydroxysuccinimide (3.2 g, 27.8 mmol) and EDC·HCl (7.9 g, 41.2 mmol) were added over ice water bath. The reaction was stirred at r.t. for 3.5 hours, washed with brine, dried over anhydrous sodium sulfate, filtered and concentrated to give compound 387 (14.0 g), which was used directly in the next step. MS-ESI (m/z): calcd. for C₄₅H₇₅N₆O₂₀ [M+H]⁺ 1019.50; found, 1019.58.

### Example 349. Synthesis of (2S,4R)-4-((tert-butoxycarbonyl)amino)-5-(3-((S)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazahexatetracontanamido)-4-hydroxyphenyl)-2-methylpentanoic acid (388)

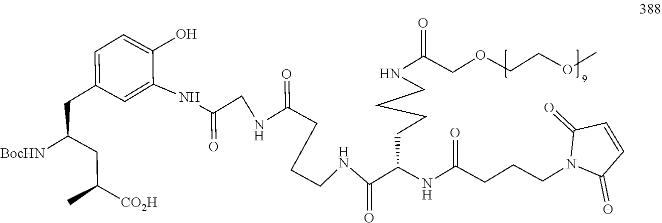

Compound 387 (14.0 g, 13.7 mmol) and compound 77 (4.2 g, 12.3 mmol) were dissolved in THF (150 mL), stirred at 25° C. for 8 hours, concentrated and diluted with ethyl acetate, washed with water. The aqueous phase was saturated with solidum chloride, extracted with dichloromathane twice. The combined organic phases were dried over anhydrous sodium sulfate, filtered and concentrated. The residue was purified by silica gel column (MeOH/CH₂Cl₂) to give compound 388 (7.6 g, 50% yield over 2 steps). MS-ESI (m/z): calcd. for C₅₈H₉₆N₇O₂₂ [M+H]⁺ 1242.65; found, 1242.65.

### Example 350. Synthesis of (2S,4R)-4-amino-5-(3-((S)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazahexatetracontanamido)-4-hydroxyphenyl)-2-methylpentanoic acid (389)

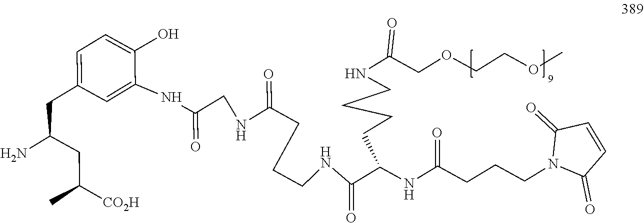

Compound 388 (0.50 g, 0.40 mmol) was dissolved in DCM (10 mL) and TFA (5 mL). The reaction was stirred for 1 hour, and then concentrated to give compound 389 (0.46 g), which is used directly in the next step. MS-ESI (m/z): calcd. for C₅₃H₈₈N₇O₂₀ [M+H]⁺ 1142.60; found, 1142.62.

### Example 351. Synthesis of (2S,4R)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-5-(3-((S)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazahexatetracontanamido)-4-hydroxyphenyl)-2-methylpentanoic acid (390)

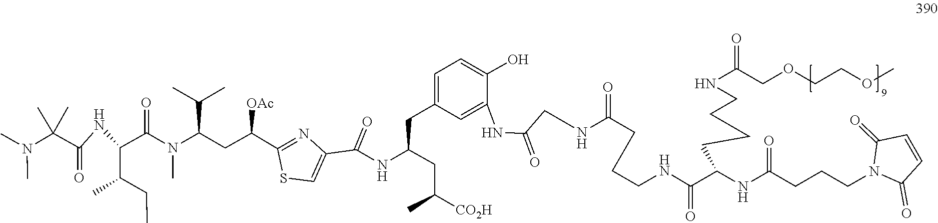

Compound 389 (0.46 g, 0.40 mmol) and Tub-1 (0.30 g, 0.44 mol) were dissolved in DMF (5 mL) and N,N-diisopropylethylamine (0.52 g, 4.0 mol) was added over ice-salt bath. The reaction was stirred at r.t. for 2 hours and concentrated. The residue was dissolved in dichloromethane (10 mL) and formic acid (0.5 mL), concentrated again. The residue was purified by preparative HPLC to give compound 390 (0.26 g, 40% yield). MS-ESI (m/z): calcd. for C₇₈H₁₂₈N₁₁O₂₅S [M+H]⁺ 1650.87; found, 1650.87.

### Example 352. Synthesis of (5S,8S,11S)-tert-butyl 11-(((benzyloxy)carbonyl)amino)-5,8-dimethyl-4,7,10,17-tetraoxo-19,22,25,28,31,34,37,40,43,46-decaoxa-2,3,6,9,16-pentaazaheptatetracontan-1-oate (392)

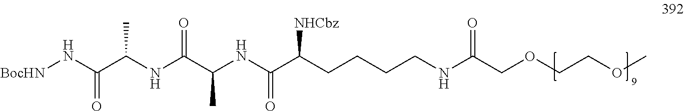

To a solution of compound 391 (2.67 g, 3.00 mmol) and tert-butyl carbazate (0.48 g, 3.60 mmol) in DCM (20 mL), EDC·HCl (0.69 g, 3.60 mmol) was added, the reaction was stirred for 1 h, washed with brine, dried over anhydrous sodium sulfate, filtered, concentrate and purified by silica gel column (MeOH/CH₂Cl₂) to give the title compound (2.56 g, 85% yield). MS-ESI (m/z): [M+H]⁺ calcd. for C₄₆H₈₀N₆O₁₈, 1005.55; found, 1005.65.

### Example 353. Synthesis of (5S,8S,11S)-tert-butyl 11-amino-5,8-dimethyl-4,7,10,17-tetraoxo-19,22,25,28,31,34,37,40,43,46-decaoxa-2,3,6,9,16-pentaazaheptatetracontan-1-oate (393)

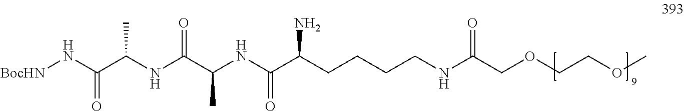

To a solution of compound 392 (2.56 g, 2.55 mmol) in methanol (20 mL), 10 wt % Pd/C (0.30 g) was added, and the reaction flask was evacuated and back-filled with hydrogen for three times, and then stirred under a hydrogen balloon at r.t. for 2 hours, filtered, and concentrated to dryness to afford compound 393 (2.10 g, 94% yield). MS-ESI (m/z): [M+H]⁺ calcd. for C₃₈H₇₄N₆O₁₆, 871.52; found, 871.56.

### Example 354. Synthesis of (5S,8S,11S)-tert-butyl 11-(4-(2,5-dioxo-2,5-dihydro-1H-pyrrol-1-yl)butanamido)-5,8-dimethyl-4,7,10,17-tetraoxo-19,22,25,28,31,34,37,40,43,46-decaoxa-2,3,6,9,16-pentaazaheptatetracontan-1-oate (394)

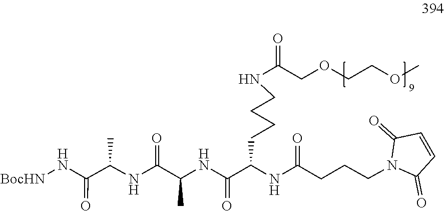

To a solution of compound 393 (2.10 g, 2.41 mmol) and 4-maleimidobutyric acid N-hydroxysuccinimide ester (0.81 g, 2.89 mmol) in DCM (25 mL) was added N-methylmorpholine (0.29 g, 2.89 mmol). The reaction was stirred at r.t. overnight, concentrated, then purified by silica gel column (MeOH/CH₂Cl₂) to give compound 394 (2.30 g, 92% yield). MS-ESI (m/z): calcd. for C₄₆H₈₁N₇O₁₉ [M+H]⁺ 1036.56; found, 1037.20.

### Example 355. Synthesis of N—((S)-5-(4-(2,5-dioxo-2,5-dihydro-1H-pyrrol-1-yl)butanamido)-6-(((S)-1-(((S)-1-hydrazinyl-1-oxopropan-2-yl)amino)-1-oxopropan-2-yl)amino)-6-oxohexyl)-2,5,8,11,14,17,20,23,26,29-decaoxahentriacontan-31-amide (395)

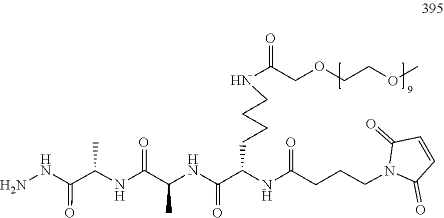

A solution of compound 394 (0.52 g, 0.50 mmol) in DCM (10 mL) was stirred with TFA (5 mL) for 30 min. and then concentrated to give a crude product (0.45 g), which is used directly in the next step. MS-ESI (m/z): calcd. for C₄₁H₇₃N₇O₁₇ [M+H]⁺ 936.51; found, 936.55.

### Example 356. Synthesis of (1R,3R)-3-((2S,3S)-2-(2-(dimethylamino)-2-methylpropanamido)-N,3-dimethylpentanamido)-1-(4-(((37S,40S,43S,48S,50R)-37-(4-(2,5-dioxo-2,5-dihydro-1H-pyrrol-1-yl)butanamido)-40,43,48-trimethyl-31,38,41,44,47-pentaoxo-51-phenyl-2,5,8,11,14,17,20,23,26,29-decaoxa-32,39,42,45,46-pentaazahenpentacontan-50-yl)carbamoyl)thiazol-2-yl)-4-methylpentyl acetate (396)

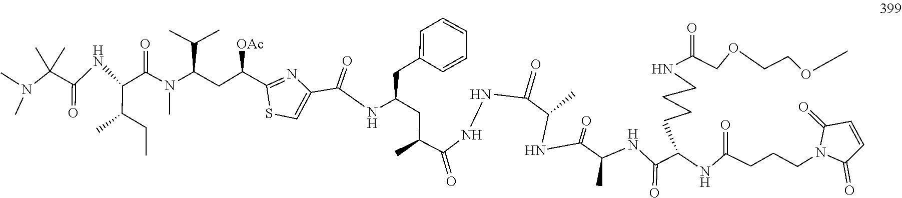 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccccc2)C[C@H](C)C(=O)NNC(=O)[C@H](C)NC(=O)[C@H](C)NC(=O)[C@H](CCCCNC(=O)COCCOC)NC(=O)CCCN2C(=O)C=CC2=O)cs1)C(C)C]

To a mixture of compound 395 (0.045 g, 0.050 mmol) and Tub-5 (0.039 g, 0.055 mmol) in DMF (10 mL) were added HATU (0.021 g, 0.055 mmol) and trimethylamine (5.5 mg, 0.055 mmol) at 0° C. The mixture was stirred at 0° C. until complete conversion, and then washed with brine (20 mL) twice, dried over Na₂SO₄ and concentrated under vacuum. The residue was purified by preparative HPLC (water/acetonitrile) to give compound 396 (0.042 g, 52% yield) as a white foam. MS-ESI (m/z): [M+2H]²⁺ calcd. for C₇₈H₁₂₈N₁₂O₂₃S, 817.45; found, 817.56.

### Example 357. Synthesis of (2S,4R)-4-(2-((6S,9R,11R)-9-isopropyl-2,3,3,8-tetramethyl-13-(4-nitrophenoxy)-4,7,13-trioxo-6-propyl-12-oxa-2,5,8-triazatridecan-11-yl)thiazole-4-carboxamido)-2-methyl-5-phenylpentanoic acid (397)

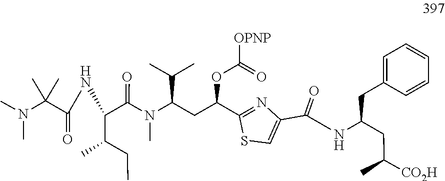

To a mixture of compound Tub-6 (0.034 g, 0.050 mmol) and 4-nitrobenzoyl chloride (0.011 g, 0.060 mmol) in 20 mL of dry dichloromethane was added N,N-DIISOPROPYLETHYLAMINE (8 mg, 0.060 mmol) at 0° C. After stirring for 30 min, the reaction mixture was loaded on a short silica gel column and eluted with MeOH/CH₂Cl₂. Fractions were combined and concentrated to give the title compound (0.030 g, 72% yield). MS-ESI (m/z): [M+H]⁺ calcd. for C₄₁H₅₆N₆O₁₀S, 825.38; found, 825.60.

### Example 358. Synthesis of N—((S)-6-(((S)-1-(((S)-1-((2-aminoethyl)amino)-1-oxopropan-2-yl)amino)-1-oxopropan-2-yl)amino)-5-(4-(2,5-dioxo-2,5-dihydro-1H-pyrrol-1-yl)butanamido)-6-oxohexyl)-2,5,8,11,14,17,20,23,26,29-decaoxahentriacontan-31-amide (398)

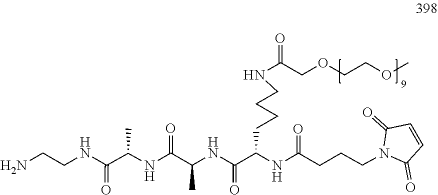

Tert-butyl ((37S,40S,43S)-37-(4-(2,5-dioxo-2,5-dihydro-1H-pyrrol-1-yl)butanamido)-40,43-dimethyl-31,38,41,44-tetraoxo-2,5,8,11,14,17,20,23,26,29-decaoxa-32,39,42,45-tetraazaheptatetracontan-47-yl)carbamate (0.043 g, 0.040 mmol) was dissolved in dichloromethane (5 mL) and treated with TFA (2.5 mL) for 30 min, and the reaction was then concentrated to give the title compound, which is used directly in the next step.

### Example 359. Synthesis of (2S,4R)-4-(2-((37S,40S,43S,51R,53R,56S)-56-((S)-sec-butyl)-37-(4-(2,5-dioxo-2,5-dihydro-1H-pyrrol-1-yl)butanamido)-53-isopropyl-40,43,54,59,59,60-hexamethyl-31,38,41,44,49,55,58-heptaoxo-2,5,8,11,14,17,20,23,26,29,50-undecaoxa-32,39,42,45,48,54,57,60-octaazahenhexacontan-51-yl)thiazole-4-carboxamido)-2-methyl-5-phenylpentanoic acid (399)

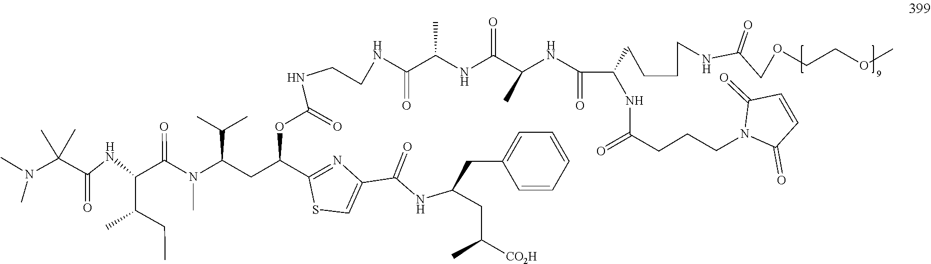

To a solution of 397 (0.030 g, 0.036 mmol) and compound 398 (0.040 mmol) in DMF (5 mL) was added N,N-diisopropylethylamine (13 mg, 0.10 mmol) at 0° C. The reaction was stirred at 0° C. for 2 hours and concentrated, then purified by preparative HPLC (water/acetonitrile) to give compound 399 (53 mg, 90% yield). MS-ESI (m/z): calcd. for C₇₉H₁₃₀N₁₂O₂₄S [M+2H]²⁺ 832.45; found, 832.56.

### Example 360. Synthesis of (1R,3R)-3-((2S,3S)-2-(2-(dimethylamino)-2-methylpropanamido)-N,3-dimethylpentanamido)-1-(4-(((37S,40S,43S,50S,52R)-37-(4-(2,5-dioxo-2,5-dihydro-1H-pyrrol-1-yl)butanamido)-40,43,50-trimethyl-31,38,41,44,49-pentaoxo-53-phenyl-2,5,8,11,14,17,20,23,26,29-decaoxa-32,39,42,45,48-pentaazatripentacontan-52-yl)carbamoyl)thiazol-2-yl)-4-methylpentyl acetate (400)

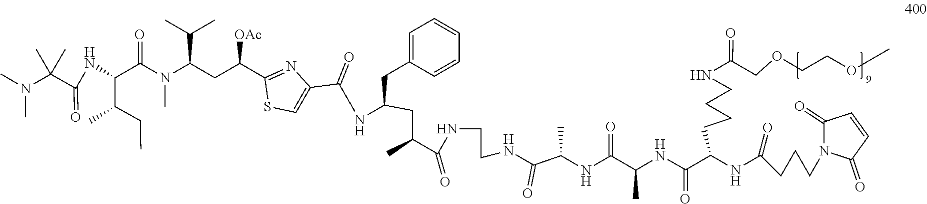

To a solution of Tub-5 (29 mg, 0.040 mmol) and compound 398 (0.040 mmol) in DMF (10 mL) cooled over an ice-water bath, were added HATU (0.19 g, 0.050 mmol) and triethylamine (10 mg, 0.10 mmol). The reaction was warmed to r.t. and stirred overnight, washed with brine, dried, concentrated, and purified by preparative HPLC (water/acetonitrile) to give a white foam (55 mg, 83% yield). MS-ESI (m/z): [M+2H]²⁺ calcd. for C₈₀H₁₃₂N₁₂O₂₃S, 831.46; found, 832.58.

### Example 361. Synthesis of (37S,40S,43S)-2,5-dioxopyrrolidin-1-yl 37-(4-(2,5-dioxo-2,5-dihydro-1H-pyrrol-1-yl)butanamido)-40,43-dimethyl-31,38,41,44-tetraoxo-2,5,8,11,14,17,20,23,26,29-decaoxa-32,39,42,45-tetraazaheptatetracontan-47-oate (401)

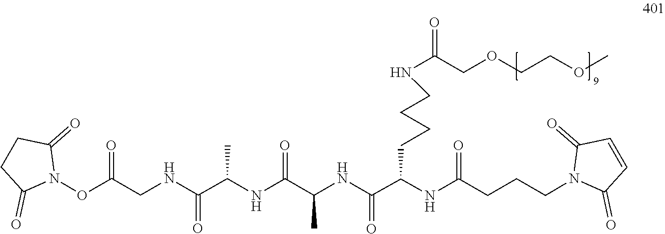

To a solution of ((S)-37-(4-(2,5-dioxo-2,5-dihydro-1H-pyrrol-1-yl)butanamido)-31-oxo-2,5,8,11,14,17,20,23,26,29-decaoxa-32-azaoctatriacontan-38-oyl)-L-alanyl-L-alanylglycine (0.98 g, 0.10 mmol) in DCM (10 mL), N-hydroxysuccinimide (14 mg, 0.12 mmol) and EDC·HCl (23 mg, 0.12 mmol) were added over ice water bath. The reaction was stirred at r.t. for 3.5 hours, washed with brine, dried over anhydrous sodium sulfate, filtered and concentrated to give compound 401 (0.11 g), which was used directly in the next step. MS-ESI (m/z): calcd. for C₄₇H₇₇N₇O₂₁ [M+H]⁺ 1076.52; found, 1077.50.

### Example 362. Synthesis of (2S,4R)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-5-(4-((37R,40R,43R)-37-(4-(2,5-dioxo-2,5-dihydro-1H-pyrrol-1-yl)butanamido)-40,43-dimethyl-31,38,41,44-tetraoxo-2,5,8,11,14,17,20,23,26,29-decaoxa-32,39,42,45-tetraazaheptatetracontanamido)phenyl)-2-methylpentanoic acid (402)

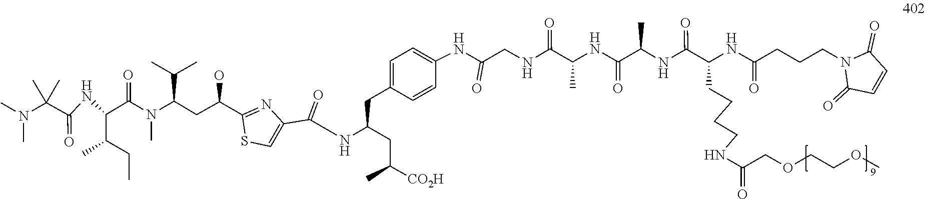

Compound 401 (0.11 g, 0.10 mmol) and Tub-7 (44 mg, 0.06 mmol) were dissolved in 2 mL of DMF, and N,N-diisopropylethylamine (13 mg, 0.10 mmol) was added. After stirring at r.t. for 2 hours, the reaction was concentrated, purified by preparative HPLC to give a light yellow liquid (62 mg, 61% yield). MS-ESI (m/z): calcd. for C₈₀H₁₃N₁₂O₂₅S [M+2H]²⁺ 846.45; found, 846.40.

### Example 363. Synthesis of benzyl ((37S,40S,43S)-40-isopropyl-43-methyl-31,38,41,44,47,50-hexaoxo-2,5,8,11,14,17,20,23,26,29-decaoxa-32,39,42,45,48-pentaazapentacontan-37-yl)carbamate (403)

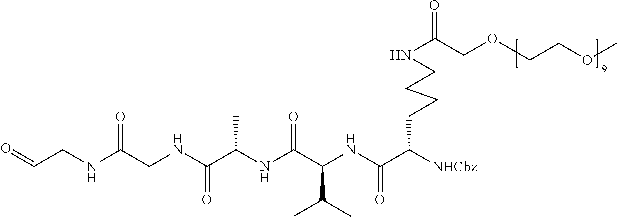

To a solution of benzyl ((37S,40S,43S)-40-isopropyl-50-methoxy-43-methyl-31,38,41,44,47-pentaoxo-2,5,8,11,14,17,20,23,26,29,51-undecaoxa-32,39,42,45,48-pentaazadopentacontan-37-yl)carbamate (102 mg, 0.10 mmol) in dichloromethane (5 mL) was added p-toluenesulfonamide (1.7 mg, 0.010 mmol) and the reaction mixture was stirred overnight, concentrated and purified by fast silica gel column (ethyl acetate/dichloromethane) to give a colorless oil (69 mg, 68% yield). MS-ESI (m/z): calcd. for C₄₇H₈₀N₆O₁₈ [M+2H]²⁺ 509.27; found, 509.26.

### Example 364. Synthesis of (2S,4R)-4-(2-((37S,40S,43S,55S,58R,60R)-37-(((benzyloxy)carbonyl)amino)-55-((S)-sec-butyl)-40,58-diisopropyl-43,51,52,52,57-pentamethyl-31,38,41,44,47,53,56,62-octaoxo-2,5,8,11,14,17,20,23,26,29,61-undecaoxa-32,39,42,45,48,51,54,57-octaazatrihexacontan-60-yl)thiazole-4-carboxamido)-5-(4-(benzyloxy)phenyl)-2-methylpentanoic acid (404)

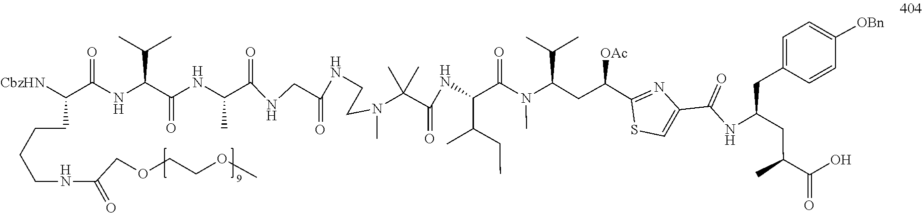

To a solution of compound 403 (69 mg, 0.068 mmol) and Tub-8 (48 mg, 0.060 mmol) in isopropyl alcohol (2.0 mL) and acetic acid (0.2 mL), sodium triacetoxyborohydride (38 mg, 0.18 mmol) at 0° C. The reaction was stirred at r.t. overnight and then concentrated and purified by preparative HPLC (water/acetonitrile) to give a white foam (92 mg, 85% yield). MS-ESI (m/z): calcd. for C₉₀H₁₄₁N₁₁O₂₅S [M+2H]²⁺ 904.99; found, 905.10.

### Example 365. Synthesis of (2S,4R)-4-(2-((37S,40S,43S,55S,58R,60R)-37-amino-55-((S)-sec-butyl)-40,58-diisopropyl-43,51,52,52,57-pentamethyl-31,38,41,44,47,53,56,62-octaoxo-2,5,8,11,14,17,20,23,26,29,61-undecaoxa-32,39,42,45,48,51,54,57-octaazatrihexacontan-60-yl)thiazole-4-carboxamido)-5-(4-hydroxyphenyl)-2-methylpentanoic acid (405)

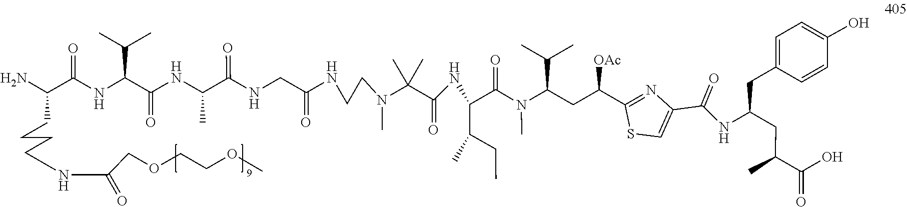

Compound 404 (92 mg, 0.051 mmol) was dissolved in methanol (5 mL), palladium on carbon (10 wt %, 10 mg) was added, and the reaction flask was evacuated and back-filled with hydrogen for three times, stirred for 4 hours. The reaction mixture was filtered, and the filtrate was concentrated to the title compound (77 mg, 96% yield). MS-ESI (m/z): [M+2H]²⁺ calcd. for C₇₅H₁₂₉N₁₁O₂₃S, 792.95; found, 973.91.

### Example 366. Synthesis of (2S,4R)-4-(2-((37S,40S,43S,55S,58R,60R)-55-((S)-sec-butyl)-37-(4-(2,5-dioxo-2,5-dihydro-1H-pyrrol-1-yl)butanamido)-60-hydroxy-40,58-diisopropyl-43, 51,52,52,57-pentamethyl-31,38,41,44,47,53,56-heptaoxo-2,5,8,11,14,17,20,23,26,29-decaoxa-32,39,42,45,48,51,54,57-octaazahexacontan-60-yl)thiazole-4-carboxamido)-5-(4-hydroxyphenyl)-2-methylpentanoic acid (406)

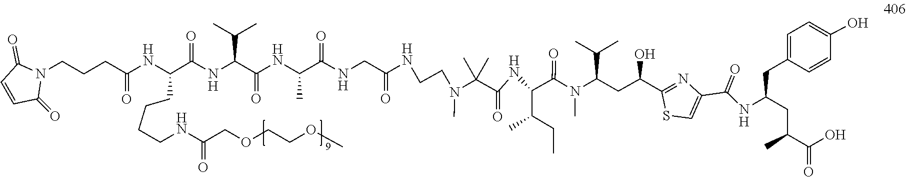

A solution of compound 405 (77 mg, 0.048 mmol) and 4-maleimidobutyric acid N-hydroxysuccinimide ester (13 mg, 0.048 mmol) in THF (1.5 mL) and PBS (pH 6.2, 1.0 mL) was stirred at r.t. overnight, concentrated, then purified by preparative HPLC (water/acetonitrile) to give compound 406 (47 mg, 58% yield). MS-ESI (m/z): calcd. for C₈₁H₁₃₄N₁₂O₂₅S [M+2H]²⁺ 854.46; found, 854.20.

### Example 367. Synthesis of (2S,4R)-benzyl 4-(2-((37S,40S,43S,53R,55R)-37-(((benzyloxy)carbonyl)amino)-52-((2S,3S)-2-(2-(dimethylamino)-2-methylpropanamido)-3-methylpentanoyl)-53-isopropyl-40,43-dimethyl-31,38,41,44,49,57-hexaoxo-2,5,8,11,14,17,20,23,26,29,56-undecaoxa-32,39,42,45,48,52-hexaazaoctapentacontan-55-yl)thiazole-4-carboxamido)-5-(4-(benzyloxy)phenyl)-2-methylpentanoate (408)

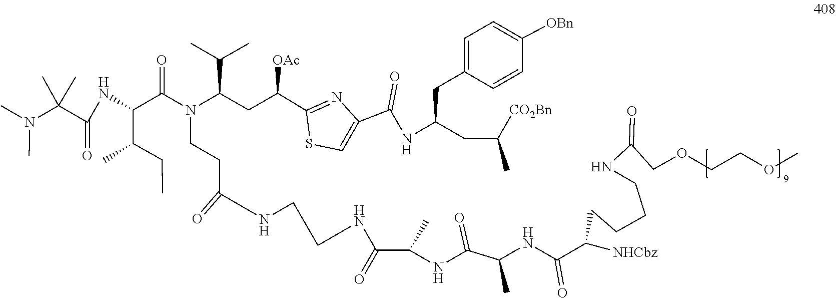

Compound 407 (50 mg, 0.05 mmol) and Tub-9 (58 mg, 0.06 mmol) were dissolved in DMF (2.5 mL), to which EDC·HCl (17 mg, 0.09 mmol) and N,N-DIISOPROPYLETHYLAMINE (19 mg, 0.15 mmol) were added, and the reaction was stirred for 0.5 hours, and then concentrated to dryness. The crude product was purified by preparative HPLC (water/acetonitrile) to give compound 408 as an oil (83 mg, 88% yield). MS-ESI (m/z): [M+2H]²⁺ calcd. for C₉₆H₁₄₅N₁₁O₂₅S, 942.00; found, 942.12.

### Example 368. Synthesis of (2S,4R)-4-(2-((37S,40S,43S,53R,55R)-52-((2S,3S)-2-(2-(dimethylamino)-2-methylpropanamido)-3-methylpentanoyl)-37-(4-(2,5-dioxo-2,5-dihydro-1H-pyrrol-1-yl)butanamido)-53-isopropyl-40,43-dimethyl-31,38,41,44,49,57-hexaoxo-2,5,8,11,14,17,20,23,26,29,56-undecaoxa-32,39,42,45,48,52-hexaazaoctapentacontan-55-yl)thiazole-4-carboxamido)-5-(4-hydroxyphenyl)-2-methylpentanoic acid (409)

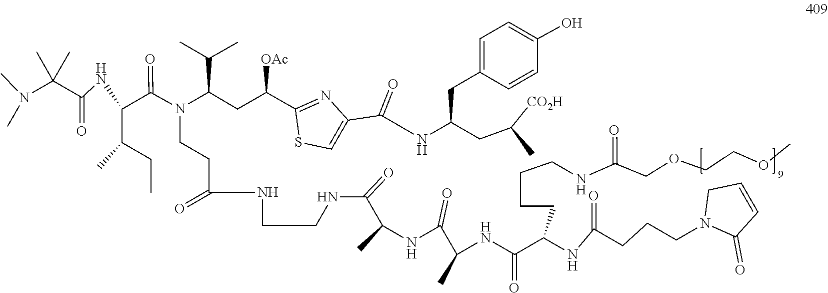

Compound 408 (83 mg, 0.044 mmol) was dissolved in methanol (5 mL), palladium on carbon (10 wt %, 10 mg) was added, and the reaction flask was evacuated and back-filled with hydrogen for three times, stirred overnight. The reaction mixture was filtered, and the filtrate was concentrated, re-dissolved in THF (1.5 mL) and PBS (pH 6.2, 1.0 mL), 4-maleimidobutyric acid N-hydroxysuccinimide ester (12 mg, 0.043 mmol) was added and stirred at r.t. overnight. The reaction was concentrated, and purified by preparative HPLC (water/acetonitrile) to give the title compound (34 mg, 45% yield). MS-ESI (m/z): calcd. for C₈₂H₁₃₄N₁₂O₂₆S [M+2H]²⁺ 867.46; found, 868.02.

### Example 369. Synthesis of 4-nitrophenyl (S)-8-(benzyloxy)-7-methoxy-2-methylene-5-oxo-2,3,11,11a-tetrahydro-1H-benzo[e]pyrrolo[1,2-a][1,4]diazepine-10(5H)-carboxylate (410)

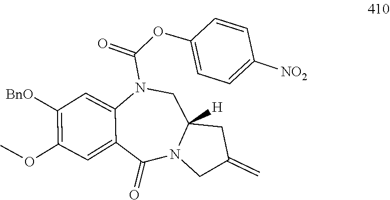 [SMILES: C=C1C[C@H]2CN(C(=O)Oc3ccc([N+](=O)[O-])cc3)c3cc(OCc4ccccc4)c(OC)cc3C(=O)N2C1]

To a solution of (S)-8-(benzyloxy)-7-methoxy-2-methylene-1,2,3,10,11,11a-hexahydro-5H-benzo[e]pyrrolo[1,2-a][1,4]diazepin-5-one (2.118 g, 6.051 mmol) in DCM (100 mL) were added DIPEA (0.931 g, 7.201 mmol) and 4-nitrophenyl chloroformate (1.331 g, 6.601 mmol) under stirring. After the addition, the mixture was stirred at r.t. overnight, washed with water, brine, dried over anhydrous sodium sulfate, filtered and concentrated. The crude product was purified by column chromatography (EA/DCM=15%-45%) to give the title compound (2.618 g, 84% yield). C₂₈H₂₆N₃O₇ [M+H]⁺ 516.177; found, 516.195.

### Example 370. Synthesis of 4-((S)-2-((tert-butoxycarbonyl)amino)propanamido)benzyl (S)-8-(benzyloxy)-7-methoxy-2-methylene-5-oxo-2,3,11,11a-tetrahydro-1H-benzo[e]pyrrolo[1,2-a][1,4]diazepine-10(5H)-carboxylate (411)

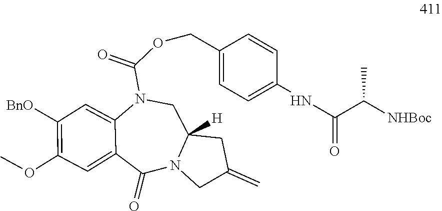 [SMILES: C=C1C[C@H]2CN(C(=O)OCc3ccc(NC(=O)[C@H](C)NC(=O)OC(C)(C)C)cc3)c3cc(OCc4ccccc4)c(OC)cc3C(=O)N2C1]

A solution of compound 410 (0.70 g, 1.36 mmol) in anhydrous THF (5 mL) and DMA (10 mL) was cooled to below 0° C. in an ice-salt batch. LiHMDS (2.4 mL, 1 mol/L) was added dropwise under N₂, and the reaction was kept below 0° C. for 20 minutes. A solution of tert-butyl (S)-(1-((4-(hydroxymethyl)-phenyl)amino)-1-oxopropan-2-yl)carbamate (compound 377) (1.01 g, 2.0 mmol) in 10 mL of THF was added dropwise, and the reaction was kept below 0° C. for 20 minutes, and warmed to r.t. and stirred for 4 hours. The reaction solution was diluted with DCM, washed with ammonium chloride solution, brine, and dried over anhydrous sodium sulfate, filtered and concentrated. The crude product was purified by column chromatography (EA/DCM=15%-40%) to give the title compound (0.59 g, 65% yield). MS, C₃₇H₄₃N₄O₈ [M+H]⁺ 671.31; found, 671.60.

### Example 371. Synthesis of 4-((S)-2-((tert-butoxycarbonyl)amino)propanamido)benzyl (S)-8-hydroxy-7-methoxy-2-methylene-5-oxo-2,3,11,11a-tetrahydro-1H-benzo[e]pyrrolo[1,2-a][1,4]diazepine-10(5H)-carboxylate (412)

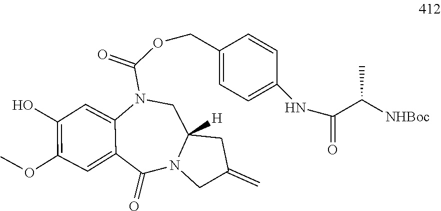 [SMILES: C=C1C[C@H]2CN(C(=O)OCc3ccc(NC(=O)[C@H](C)NC(=O)OC(C)(C)C)cc3)c3cc(O)c(OC)cc3C(=O)N2C1]

Compound 411 (1.10 g, 1.64 mmol) in DCM (15 mL) was treated with AlCl₃ (0.65 g, 4.93 mmol) and N,N-dimethylaniline (0.30 g, 2.48 mmol) at r. t. for 45 min. The mixture was diluted with DCM (15 ml), washed with 0.1 M HCl (10 ml), brine (10 ml), 5% NaHCO₃ solution and brine (10 ml), dried over anhydrous Na2SO4, concentrated and purified by column chromatography (EA/DCM=20%-40%) to give the title compound (0.685 g, 72% yield). MS, C₃₀H₃₇N₄O₈ [M+H]⁺ 581.26; found, 581.40.

### Example 372. Synthesis of (11aR)-allyl 11-((tert-butyldimethylsilyl)oxy)-8-((5-iodopentyl)oxy)-7-methoxy-2-methylene-5-oxo-2,3,11,11a-tetrahydro-1H-benzo[e]pyrrolo[1,2-a][1,4]diazepine-10(5H)-carboxylate (413)

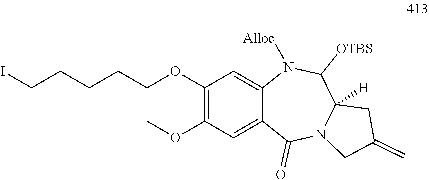

To a solution of (11aR)-allyl 11-((tert-butyldimethylsilyl)oxy)-8-hydroxy-7-methoxy-2-methylene-5-oxo-2,3,11,11a-tetrahydro-1H-benzo[e]pyrrolo[1,2-a][1,4]diazepine-10(5H)-carboxylate (1.45 g, 3.06 mmol) in acetone (100 mL) were added diiodopentane (4.86 g, 15 mmol) and potassium carbonate (0.62 g, 4.5 mmol) with stirring. After the addition, the reaction was heated under reflux for 8 hours, and after cooling, it was directly purified by column chromatography (EA/DCM=15%-30%) to give the title compound (1.87 g, 91% yield). MS, C₂₉H₄₄IN₂O₆Si [M+H]⁺ 671.20; found, 671.45.

### Example 373. Synthesis of (11aR)-allyl 8-((5-(((R)-10-(((4-((S)-2-((tert-butoxycarbonyl)amino)-propanamido)benzyl)oxy)carbonyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-((tert-butyldimethylsilyl)oxy)-7-methoxy-2-methylene-5-oxo-2,3,11,11a-tetrahydro-1H-benzo[e]pyrrolo[1,2-a][1,4]diazepine-10(5H)-carboxylate (414)

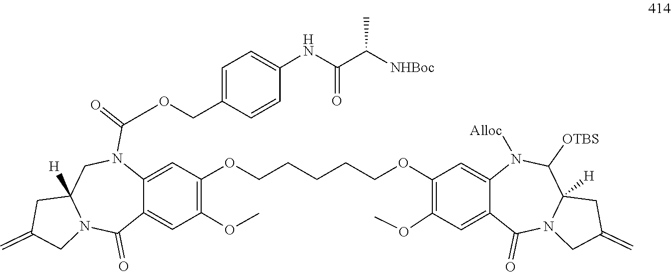

A mixture of compound 413 (0.90 g, 1.34 mmol) and compound 412 (0.80 g, 1.38 mmol) in 80 mL of acetone, and potassium carbonate (0.44 g, 3.2 mmol) was heated to reflux and stirred for 8 hours. The reaction was directly purified by a silica gel column (0-10% MeOH/DCM) to give the title compound (1.13 g, 75% yield). MS, C₅₉H₇₉N₆O₁₄Si [M+H]⁺ 1123.542; found, 1123.565.

### Example 374. Synthesis of (11aR)-allyl 8-((5-(((R)-10-(((4-((S)-2-aminopropanamido)benzyl)-oxy)carbonyl)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (415)

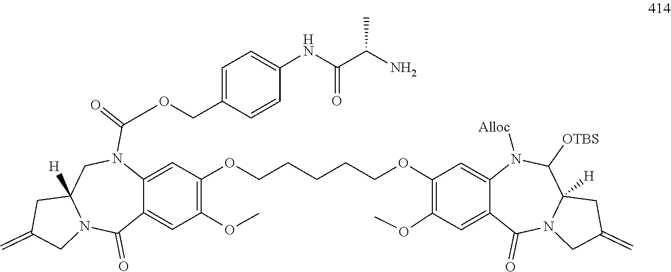

Compound 414 (0.70 g, 0.62 mmol) was dissolved in 15 mL of dioxane and cooled to 5° C., to which HCl (conc., 5 mL) was added and stirred at r.t. for 40 min. The reaction was diluted with dioxane/toluene and concentrated. The crude product was purified by a silica gel column (1:5:25, Et3N/MeOH/acetone) to give the title compound (0.41 g, 72% yield). MS, C₄₈H₅₇N₆O₁₂ [M+H]⁺ 909.40; found, 909.60.

### Example 375. Synthesis of (R)-4-((S)-2-aminopropanamido)benzyl 7-methoxy-8-((5-(((R)-7-methoxy-2-methylene-5-oxo-2,3,5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (415)

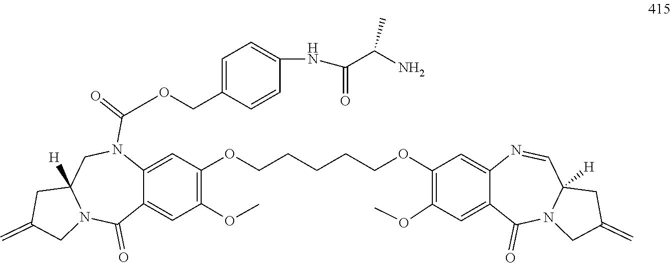

Compound 414 (115 mg, 0.126 mmol) was dissolved in 2 mL of DCM, to which pyrrolidine (19.0 mg, 0.28 mmol) and catalytic amount of Pd(PPh₃)₄ (3 mg) were added, and then stirred at r.t. for 30 min. The reaction was diluted with DMF and evaporated to remove DCM. The resulting crude product in DMF was used directly in the next step. MS-ESI (m/z): calcd. for C₄₄H₅₁N₆O₉ [M+H]⁺: 807.37; found 807.50.

### Example 376. Synthesis of 4-((37S,40S,43S,46S,49S)-37-(4-(2,5-dioxo-2,5-dihydro-1H-pyrrol-1-yl)butanamido)-40,43,46,49-tetramethyl-31,38,41,44,47-pentaoxo-2,5,8,11,14,17,20,23,26,29-decaoxa-32,39,42,45,48-pentaazapentacontan-50-amido)benzyl (S)-7-methoxy-8-((5-(((S)-7-methoxy-2-methylene-5-oxo-2,3,5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (416)

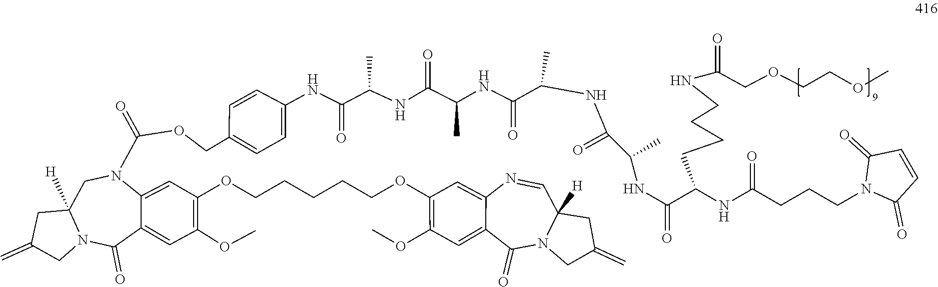

Compound 415 (˜105 mg, ˜0.131 mmol) was dissolved in 10 mL of DMF, to which compound 218 (158.0 mg, 0.145 mmol) and DIPEA (0.1 ml) were added. After stirring for 2 h, the reaction was concentrated, purified by preparative C-18 HPLC to give a white solid (118 mg, 51% yield). MS-ESI (m/z): calcd. for C₈₈H₁₂₅IN₁₂O₂₇ [M+H]⁺ 1780.87; found 1781.25.

### Example 377. Synthesis of ((((2S,5S,8S,11S,14S,22S,23S,31S,34S,37S,40S,43S)-22,23-bis(2,5-dioxo-2,5-dihydro-1H-pyrrol-1-yl)-2,5,8,11,34,37,40,43-octamethyl-4,7,10,13,16,21,24,29,32,35,38,41-dodecaoxo-14,31-bis(31-oxo-2,5,8,11,14,17,20,23,26,29-decaoxa-32-azahexatriacontan-36-yl)-3,6,9,12,15,20,25,30,33,36,39,42-dodecaazatetratetracontanedioyl)bis(azanediyl))bis(4,1-phenylene))bis(methylene) (11aS,11a′S)-bis(7-methoxy-8-((5-(((S)-7-methoxy-2-methylene-5-oxo-2,3,5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate) (417)

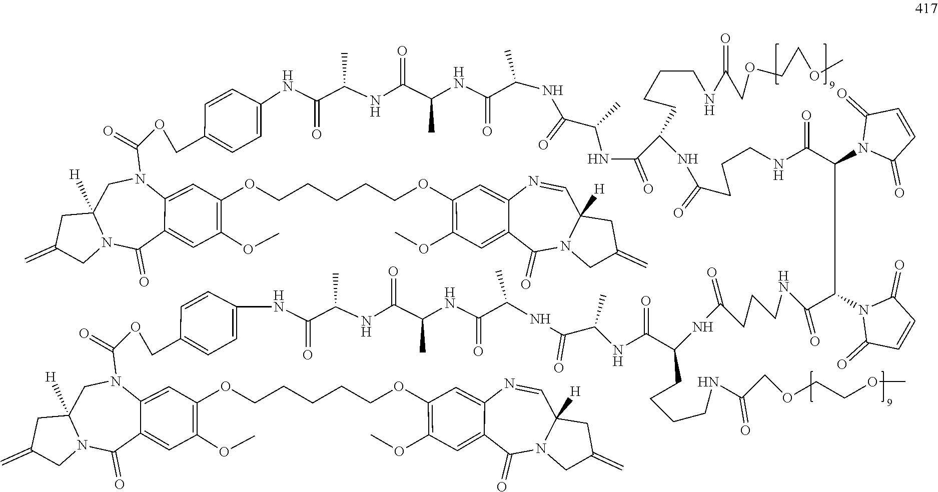

A solution of compound 136 (73 mg, 0.035 mmol), HATU (40 mg, 0.105 mmol) in DMF (1 mL) was stirred at r.t. for 15 min. and then the crude product of 415 (60 mg, 0.074 mmol) in DMF was added to the reaction solution, followed by DIPEA (5 mg, 0.039 mmol). After stirring at r.t. for 90 min., the reaction mixture was directly purified by preparative HPLC to give a pale yellow solid (61 mg, 47% yield). MS-ESI (m/z): calcd. for C₁₈₀H₂₅₃N₂₆O₅₆ [M+H]⁺: 1837.3875; found 1837.3960.

### Example 378. Preparation of the BCMA Conjugate Via the Homogeneous Conjugation Reaction

A zinc amino complex (e.g. Zinc 2-methylpropane-1, 2-diamine chloride complex) (in 10-60 mM, 1.0-5.0 eq. of an antibody used) and TCEP (in 100 mM, 2.5-4.5 eq. of an antibody used) were added in sequence to a solution containing the BCMA antibody (10-30 mg/mL, in 20 mM PBS, pH 5.5-7.5) at 2-8° C. After incubation at 2-8° C. for 12-16 h (overnight), a payload/linker complex (100-200 mM, 2.0-8.0 eq. of the antibody used) was introduced and incubated for further 2-4 h at 2-8° C. After the incubation, cystine or 4-(azidomethyl)benzoic acid (100-200 mM, 4.0-8.0 eq. of the antibody) was added to the to deplete the excess TCEP, cysteine (100-200 mM, 2.0-6.0 eq. of the antibody) was added to deplete the excess payload, EDTA (100-200 mM, 4.0-6.0 eq. of the antibody) was added to trap zinc, and DHAA (100-200 mM, 8.0-30.0 eq. of the antibody) was added to oxidize (re-bridge link) the free thiol groups in the antibody. The reaction mixture was finally purified using a de-salting column (Zeba Spin Desalting Columns, 40K MWCO), or UF/DF, or ion exchange chromatography, and drug/antibody ratio (DAR) were analyzed using HIC-HPLC or HPLC-MS.

The structures of the conjugates that were prepared by both the traditional conjugation process and the homogeneous conjugation process are illustrated below:

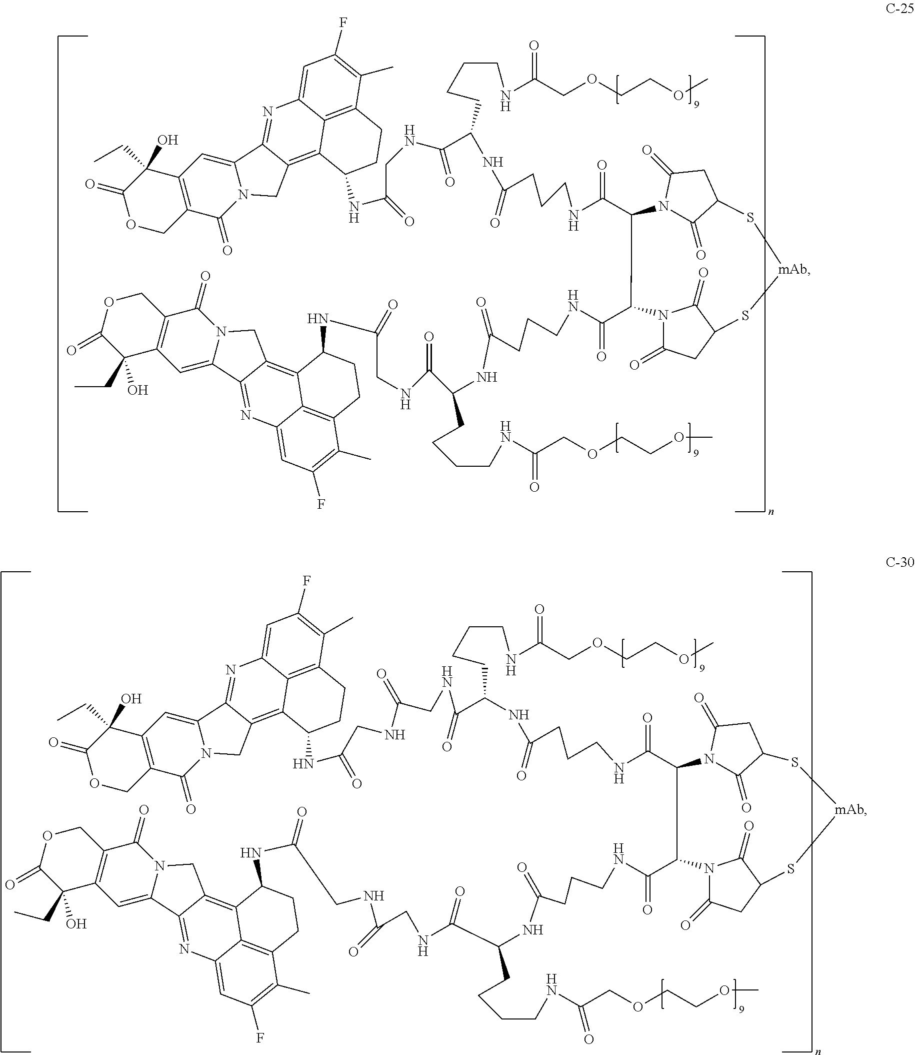

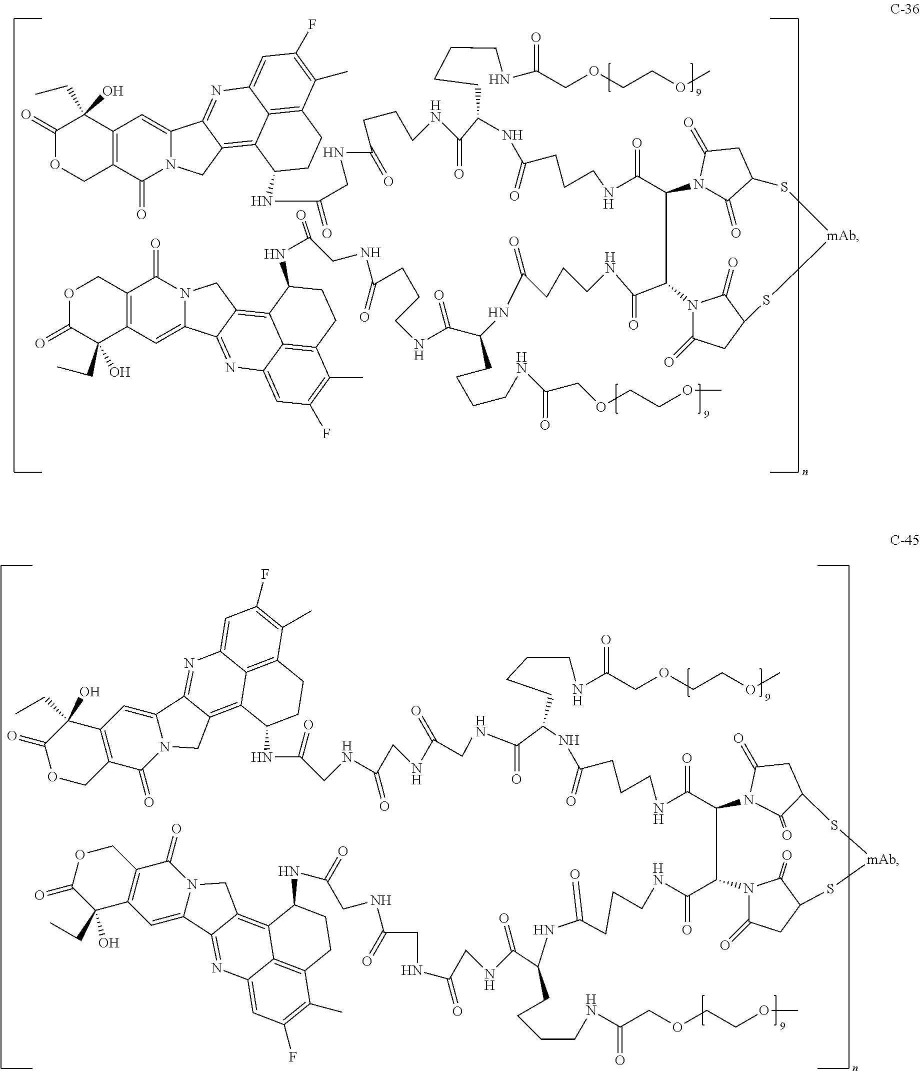

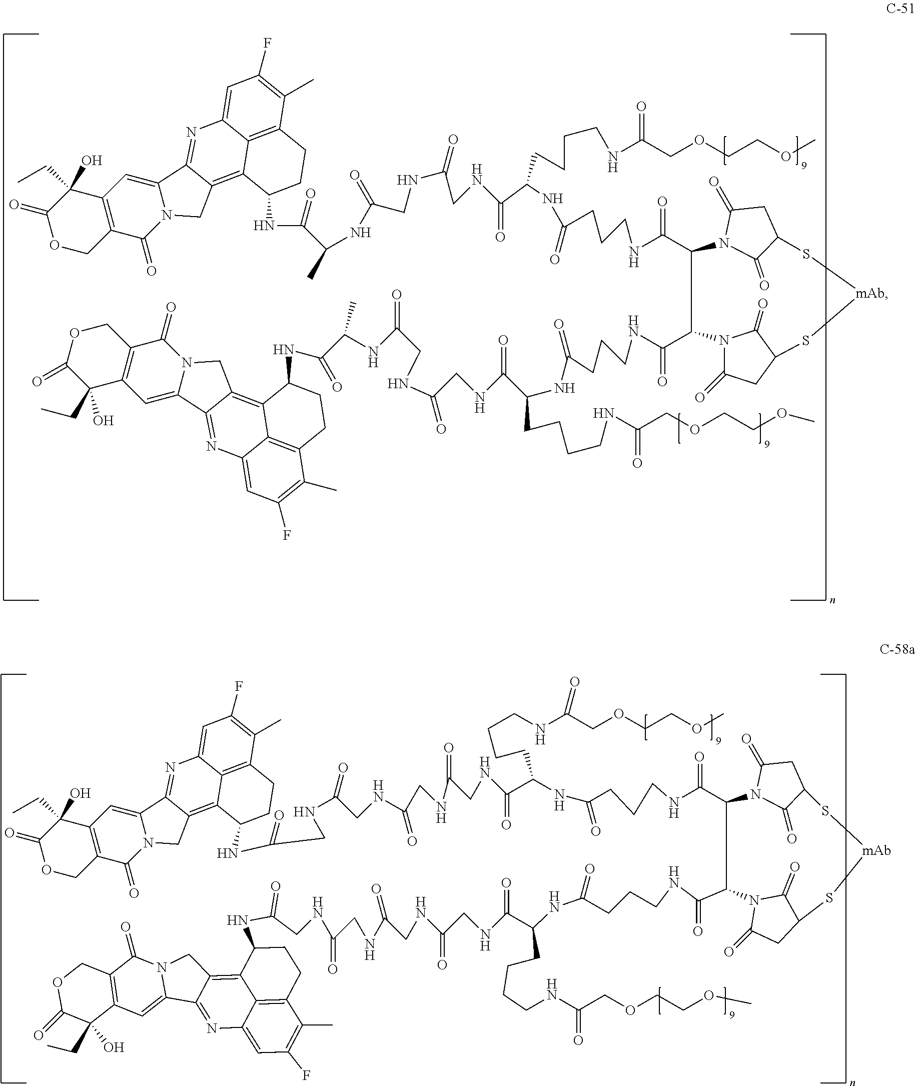

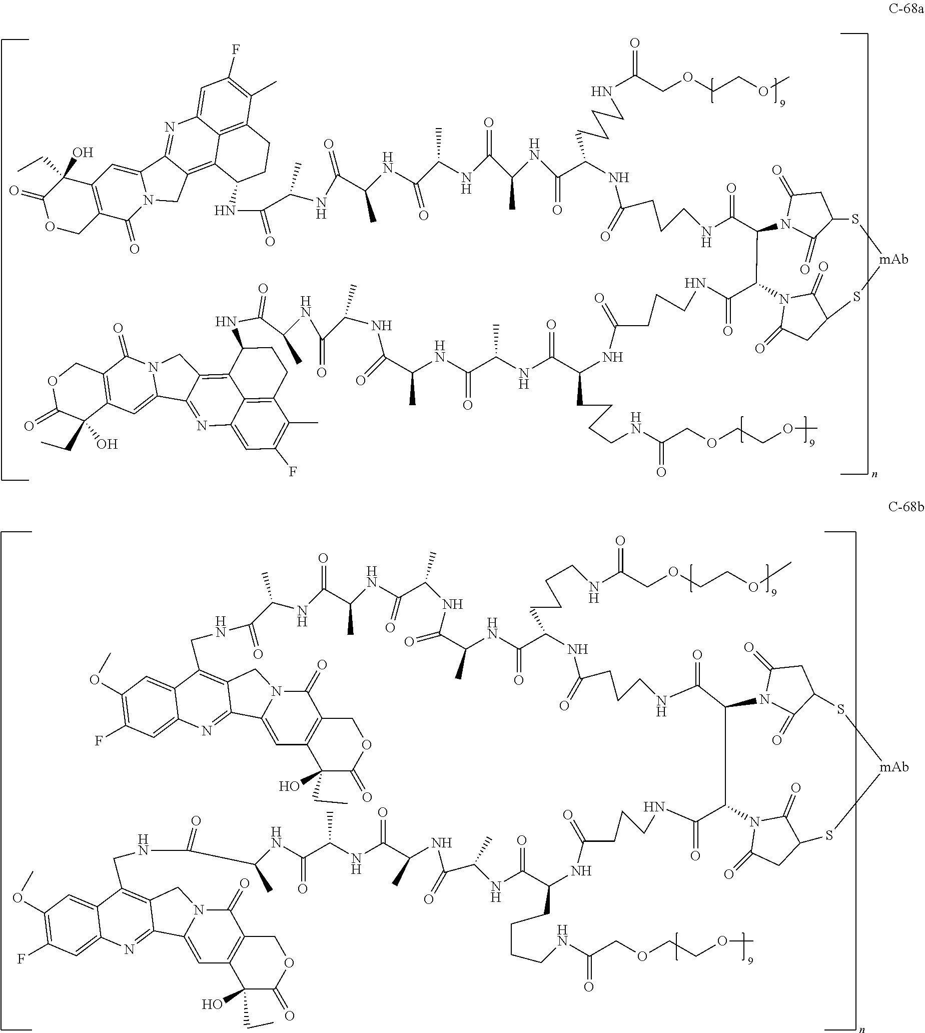

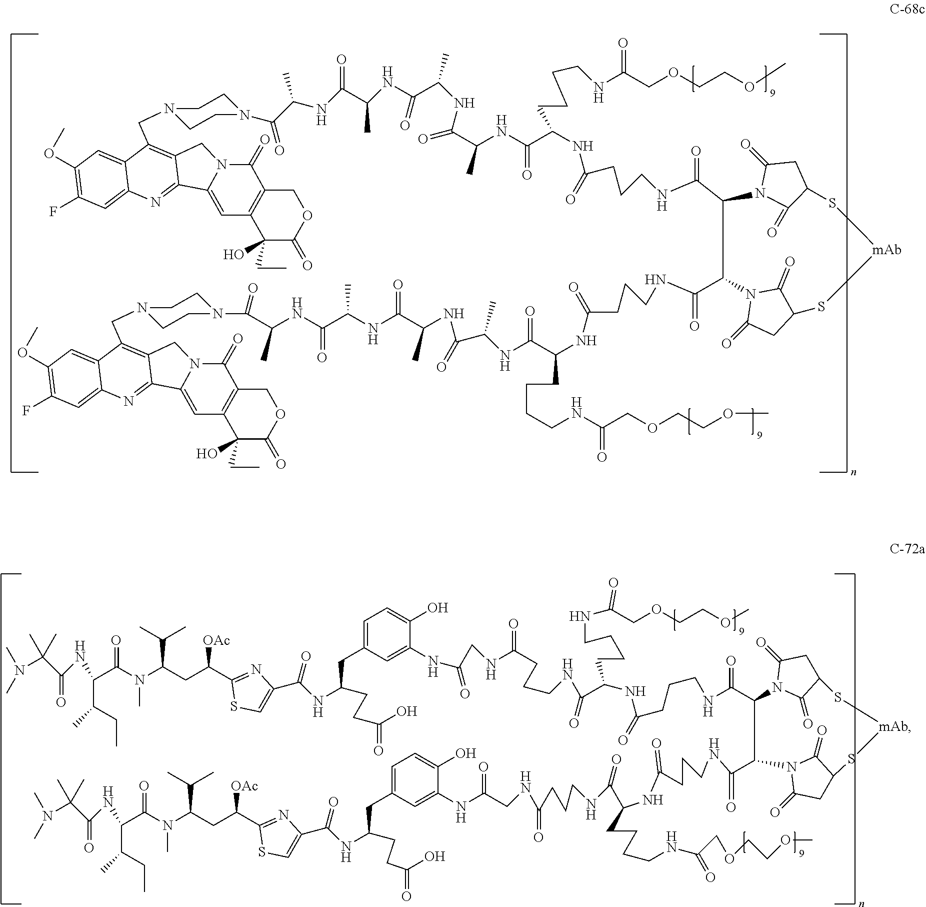

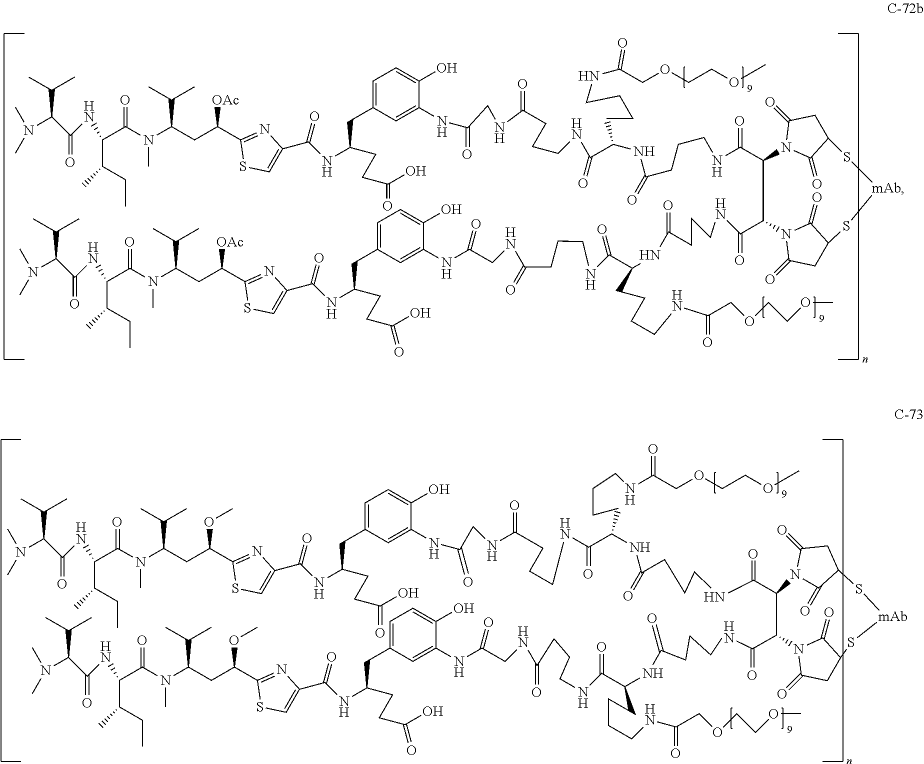

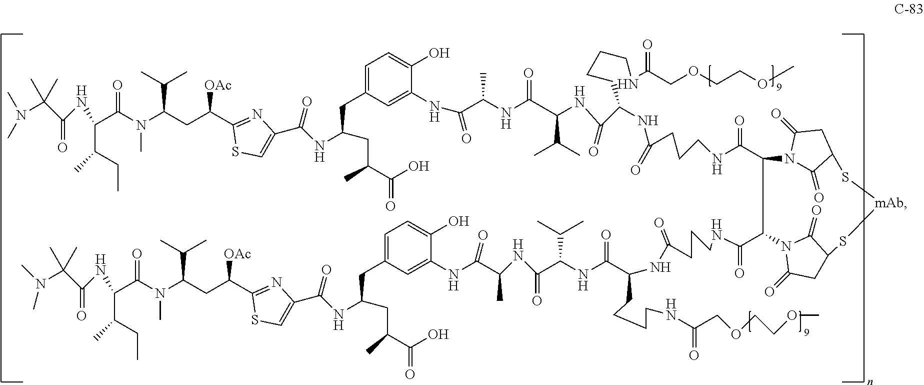

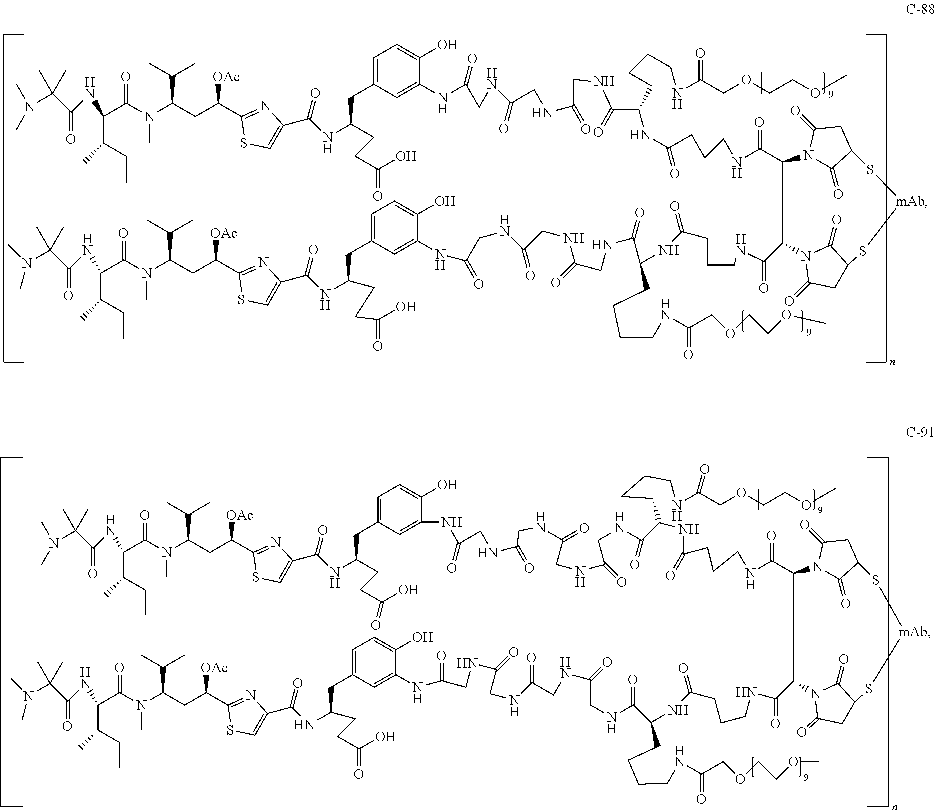

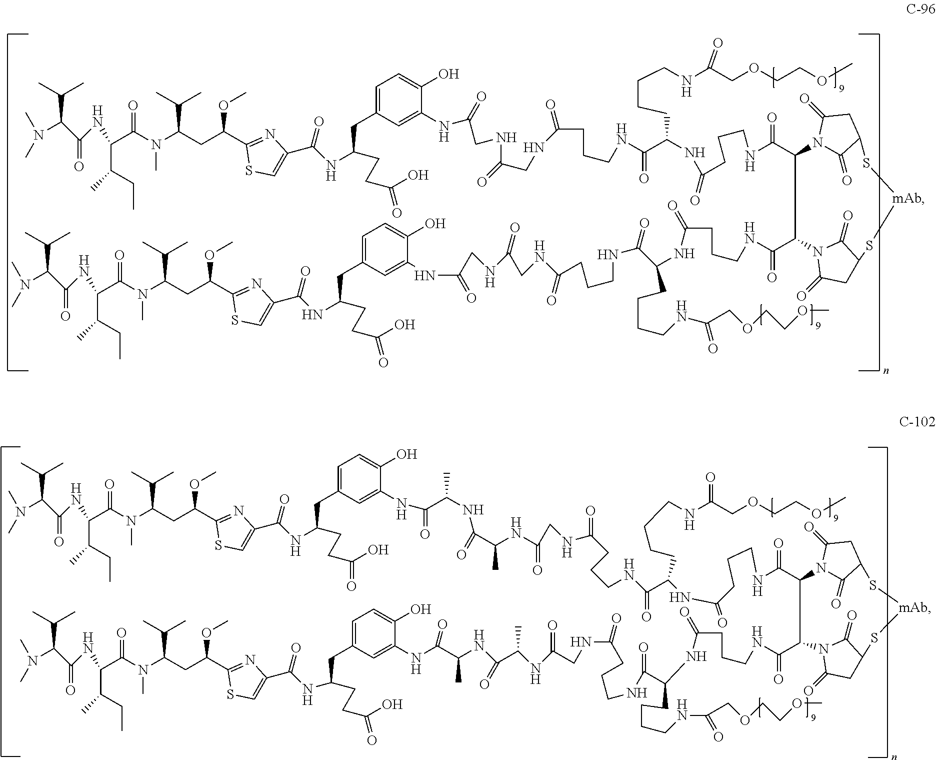

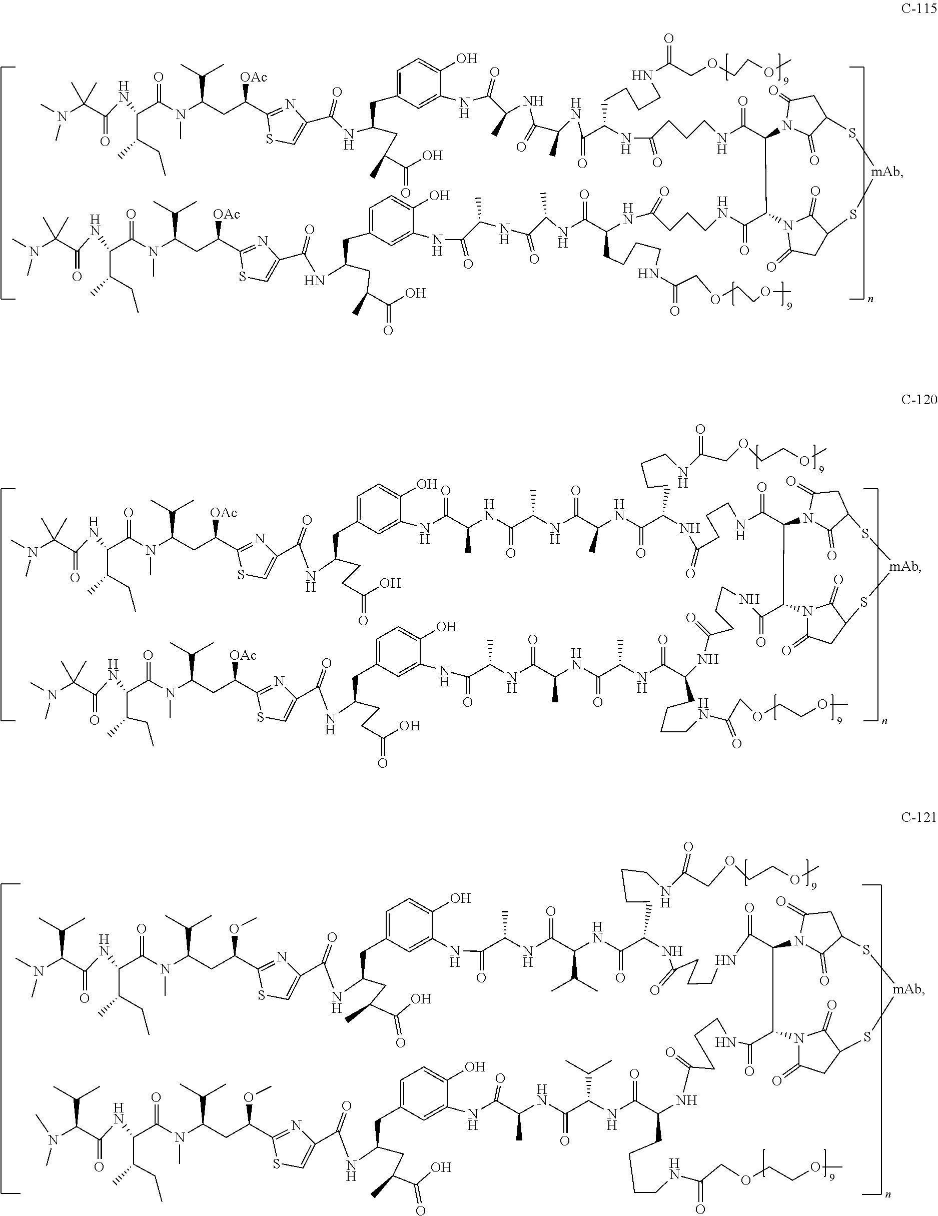

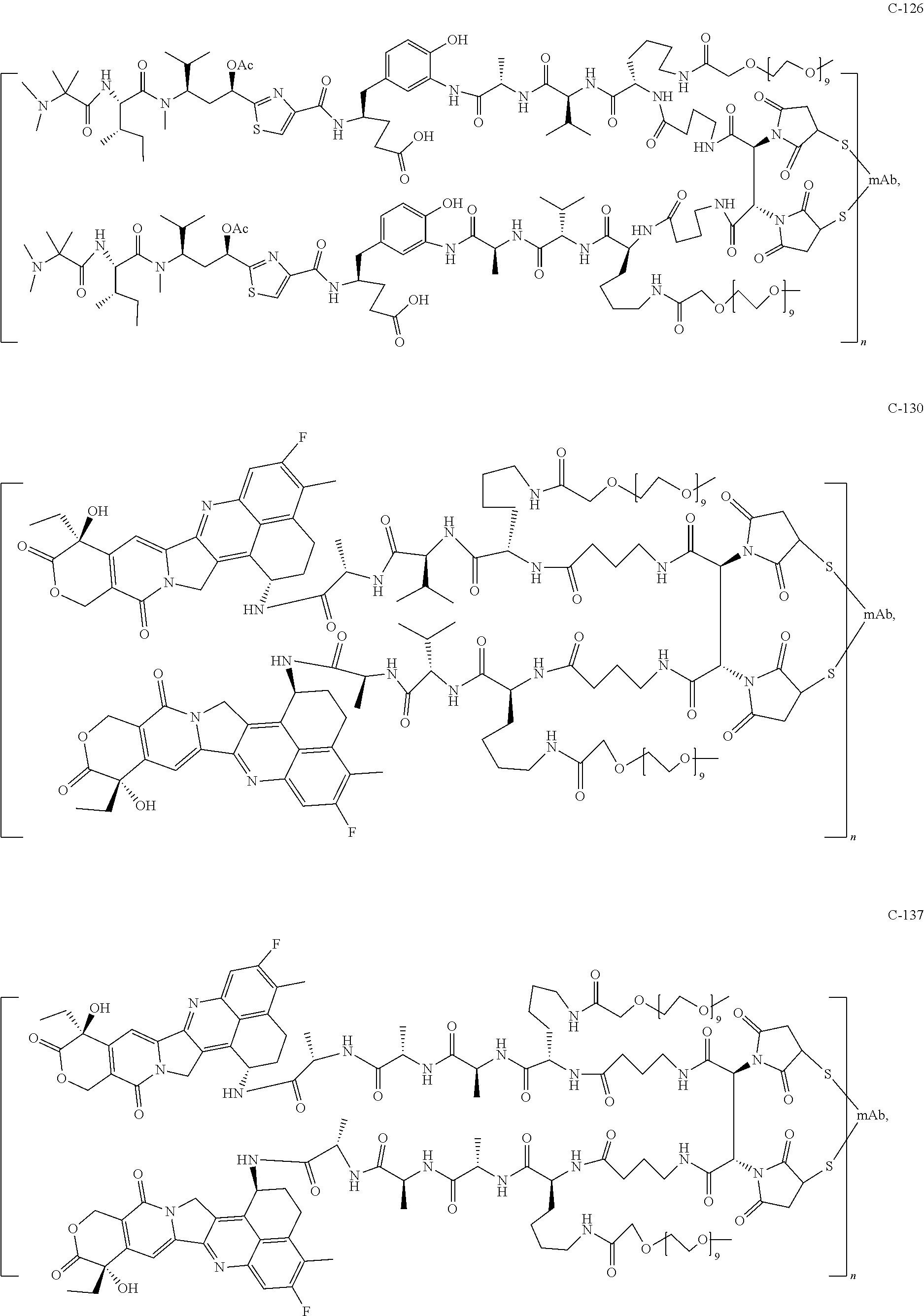

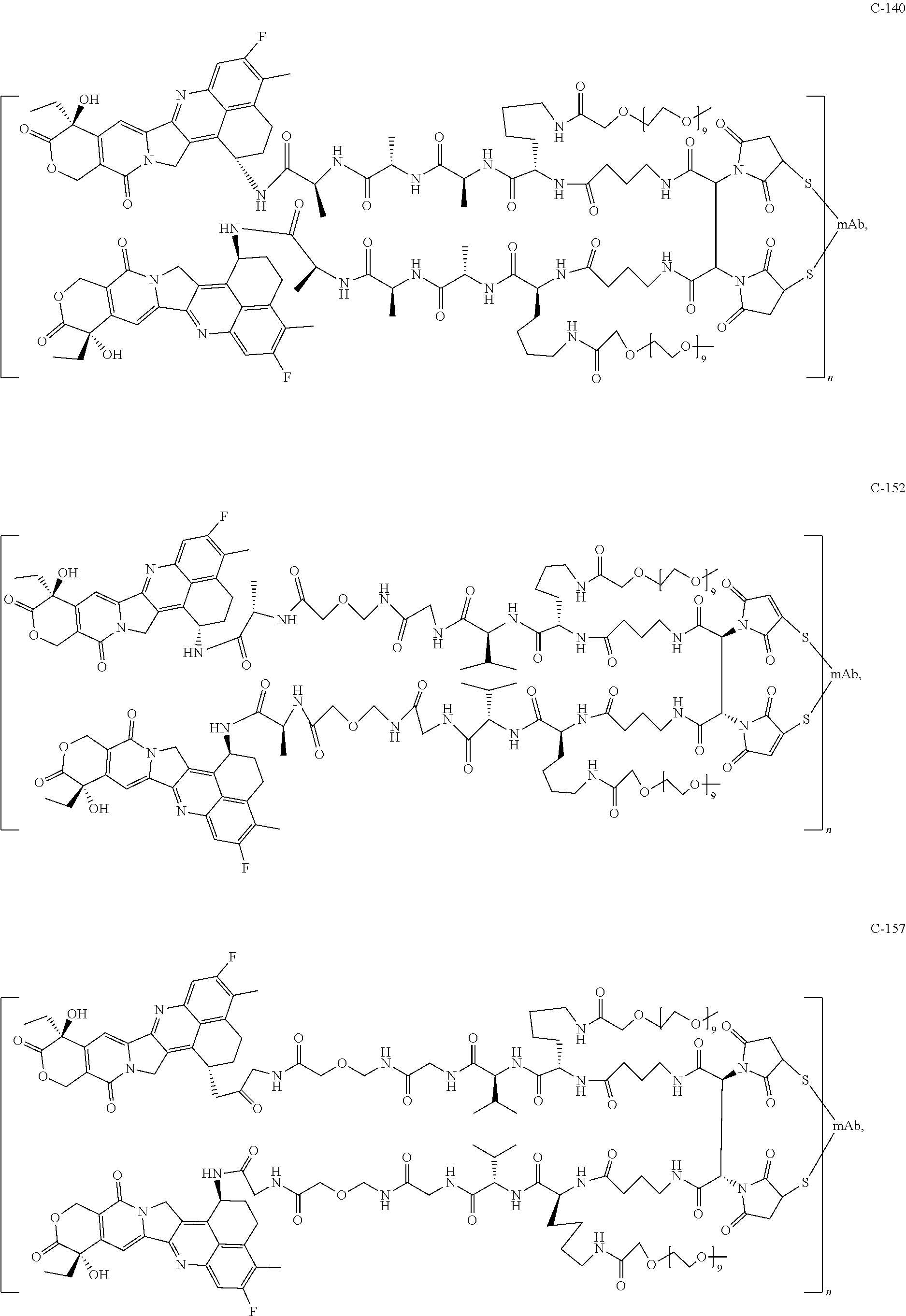

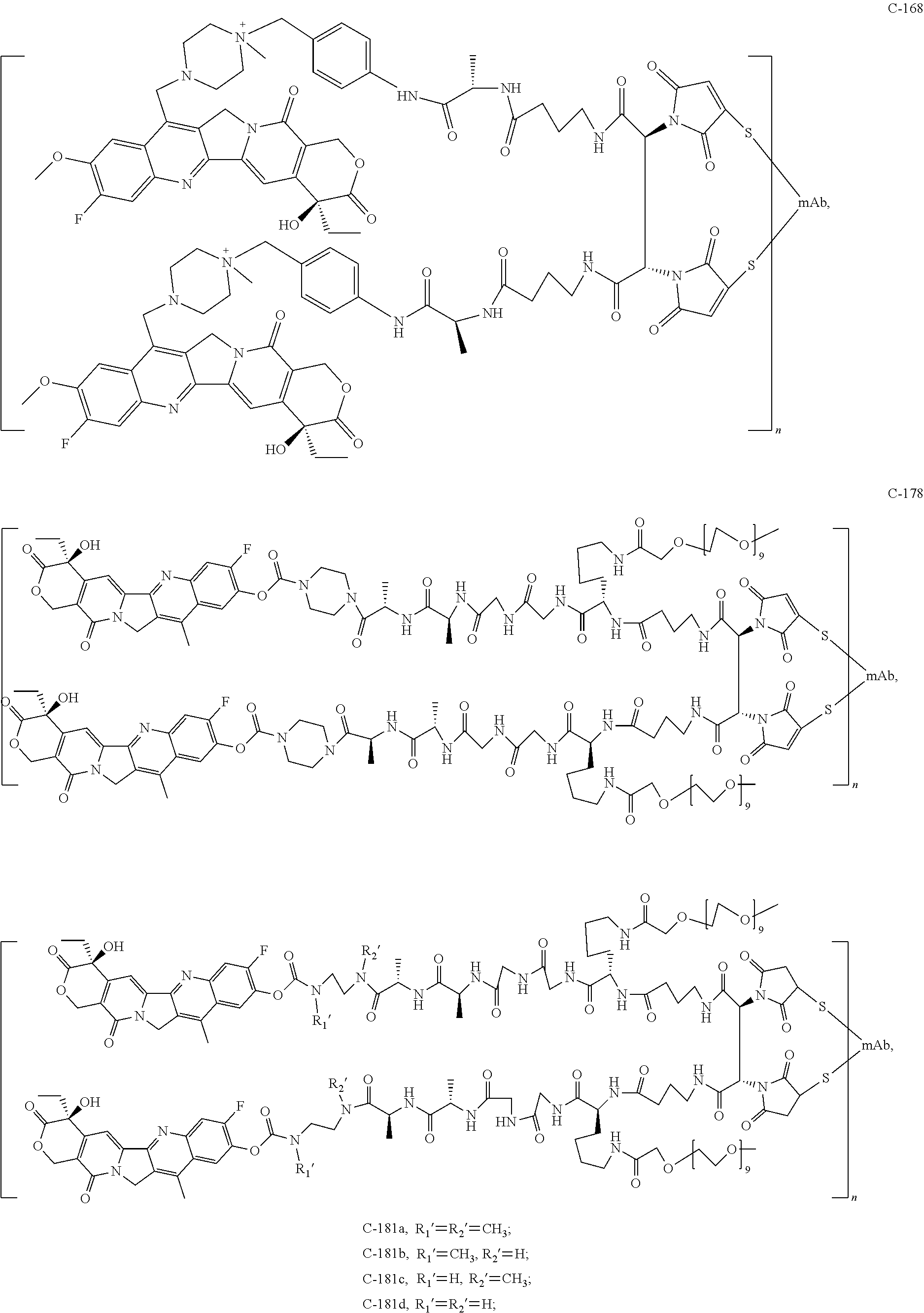

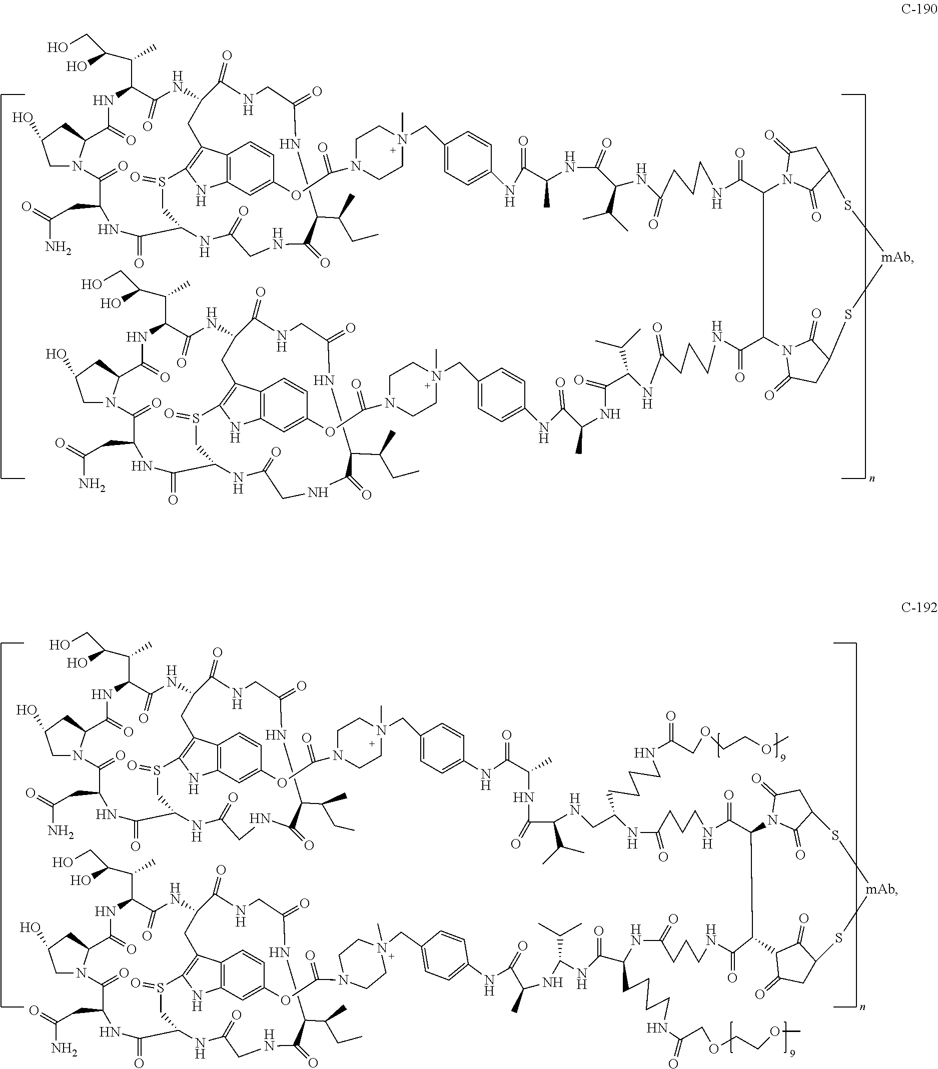

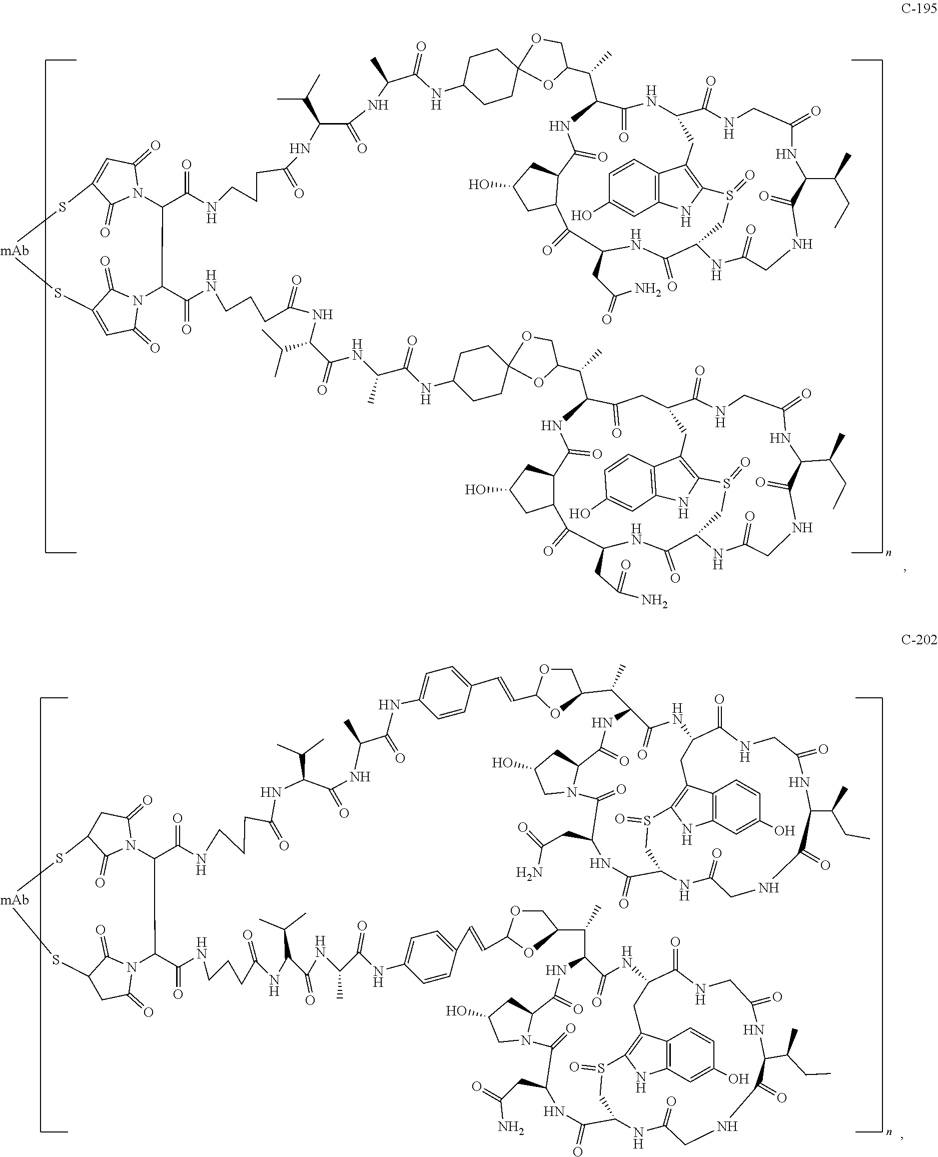

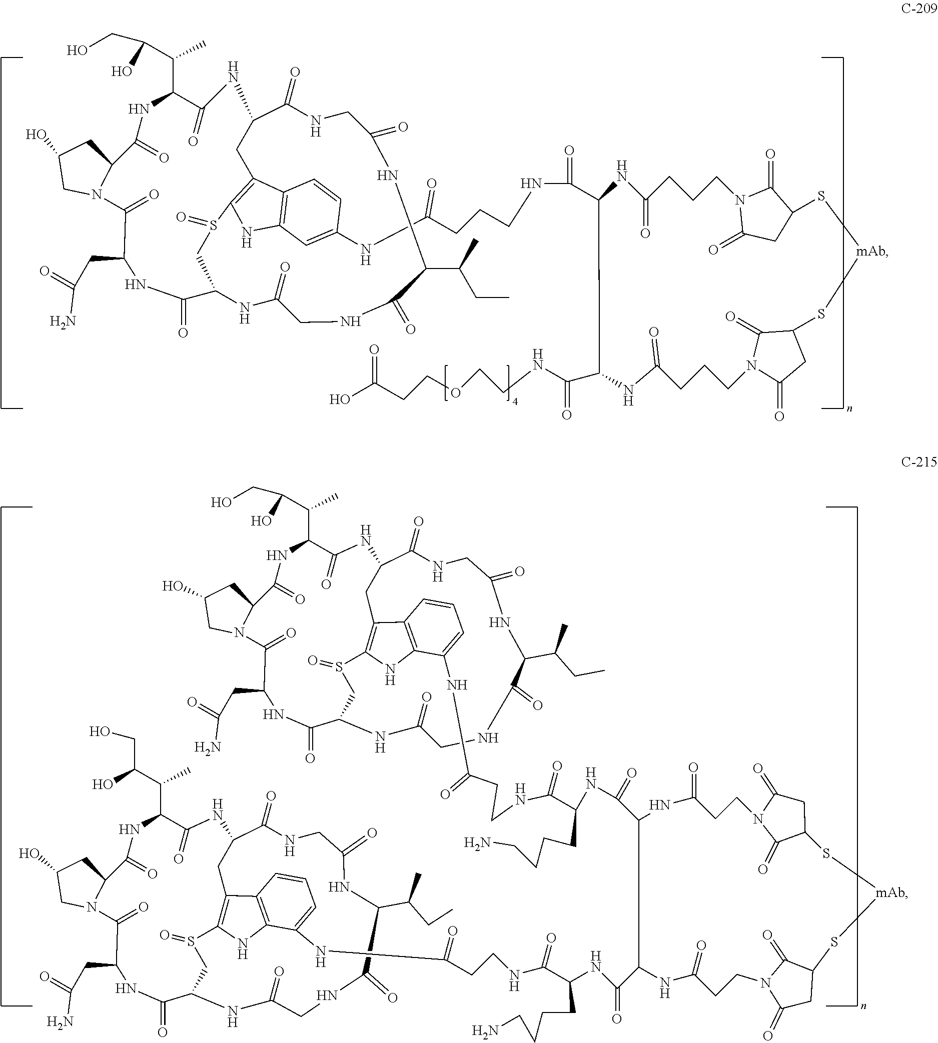

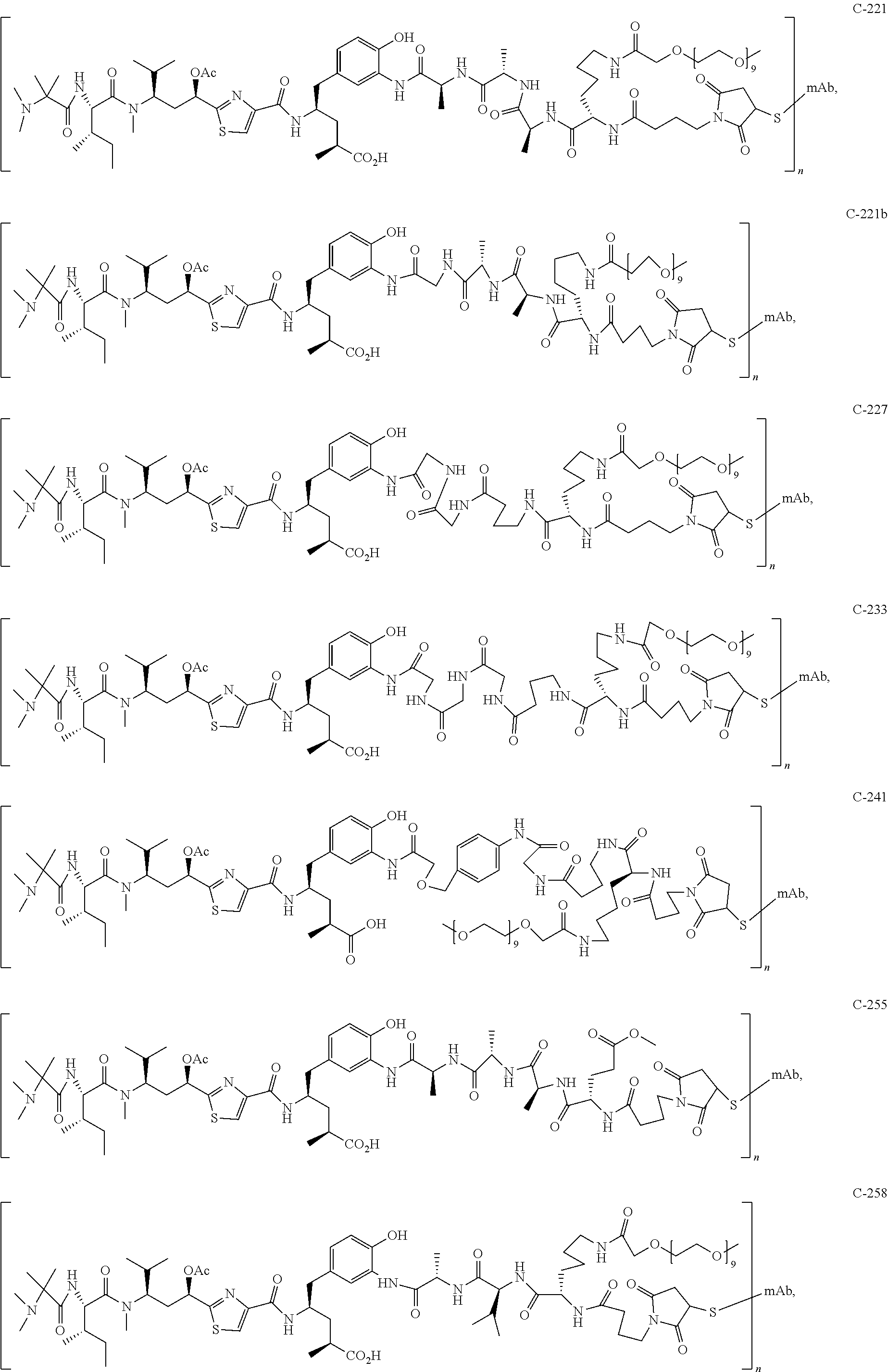

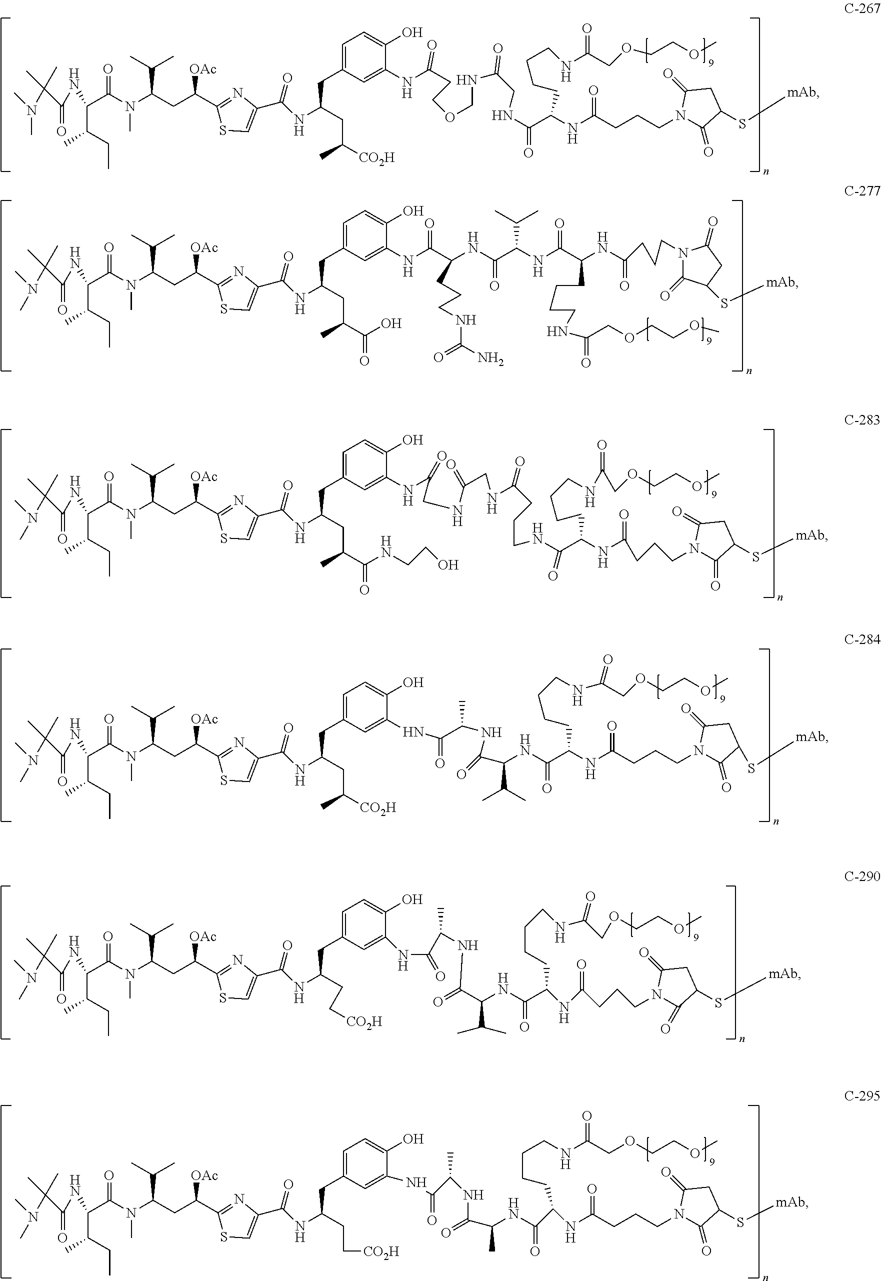

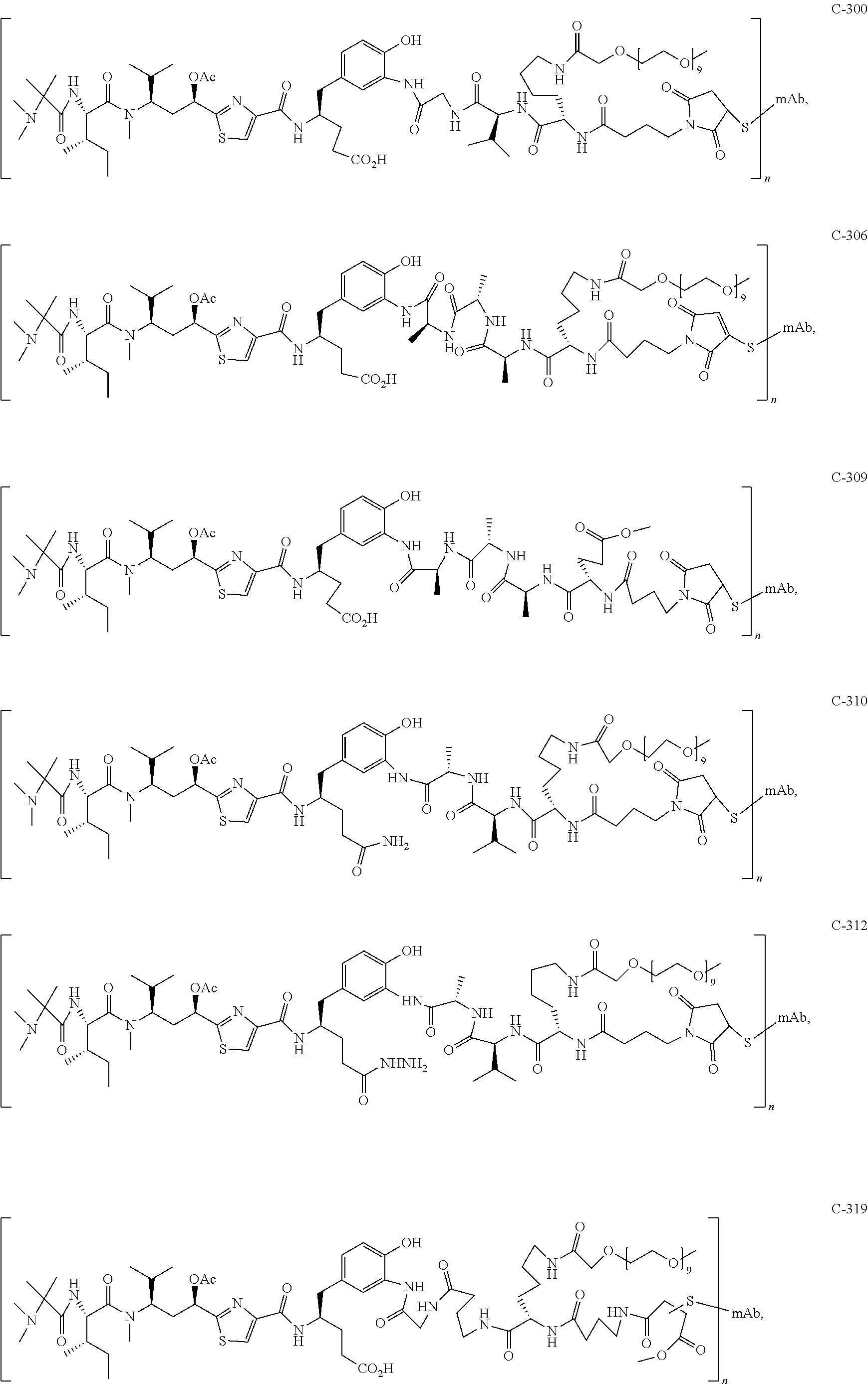

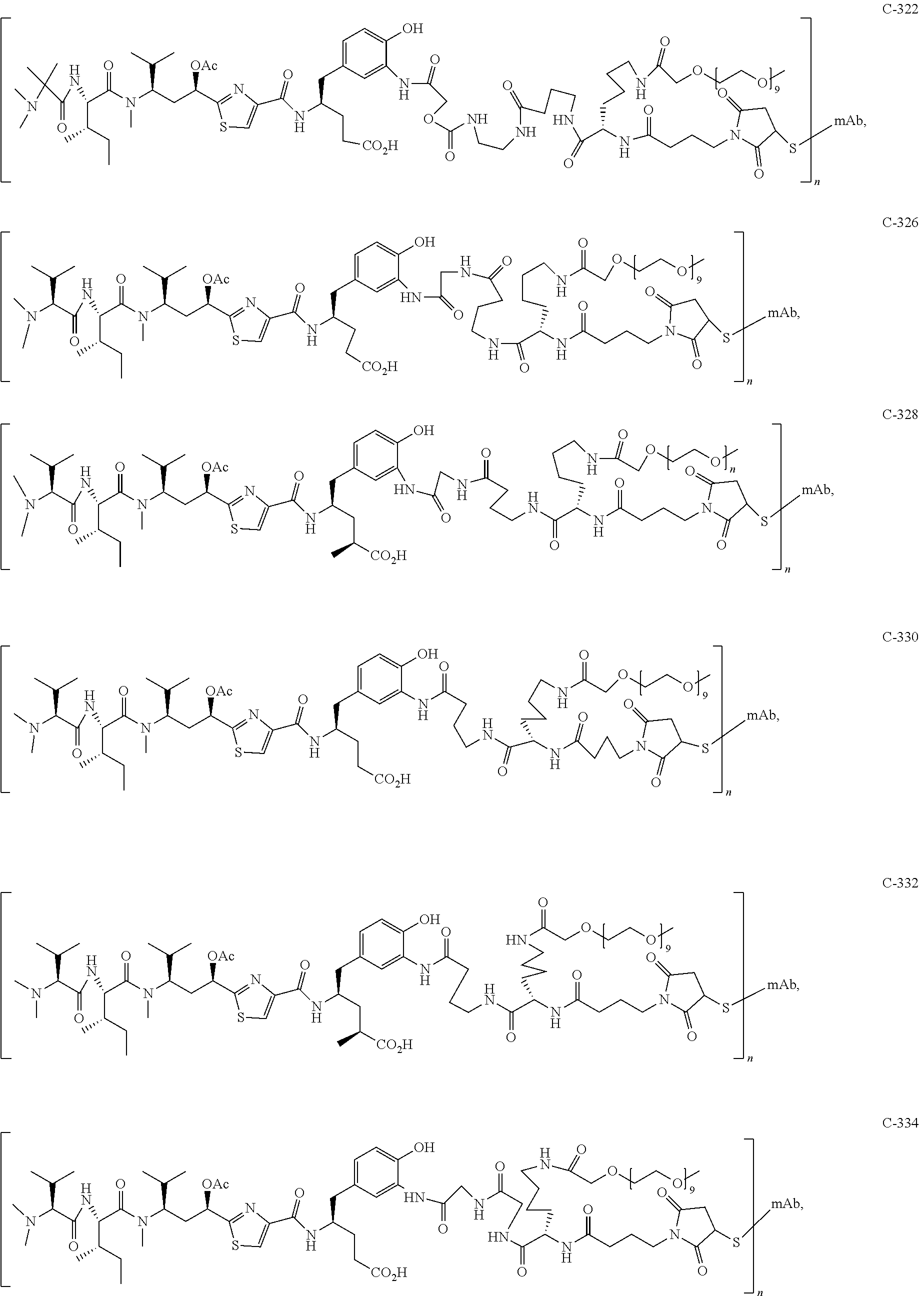

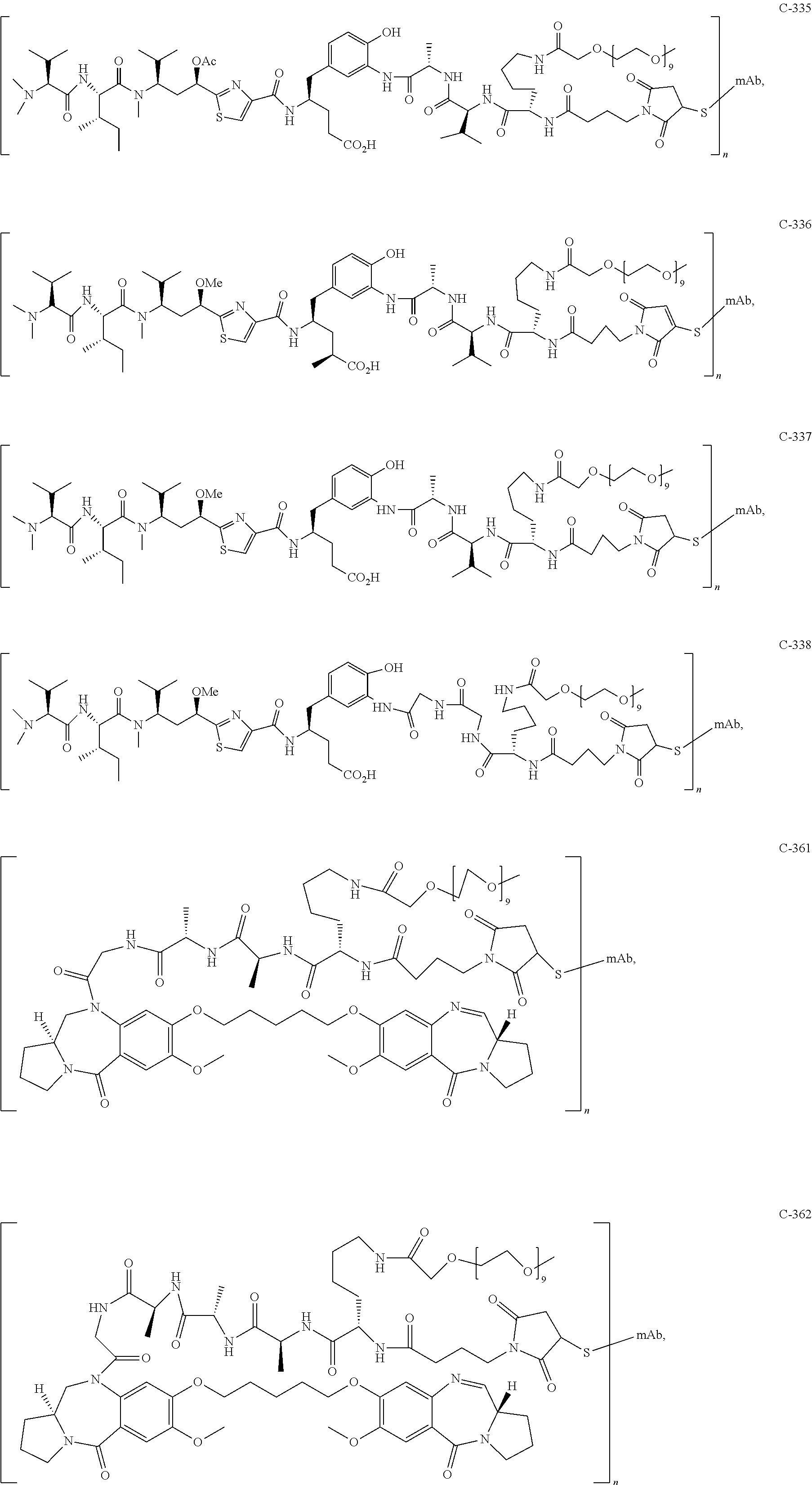

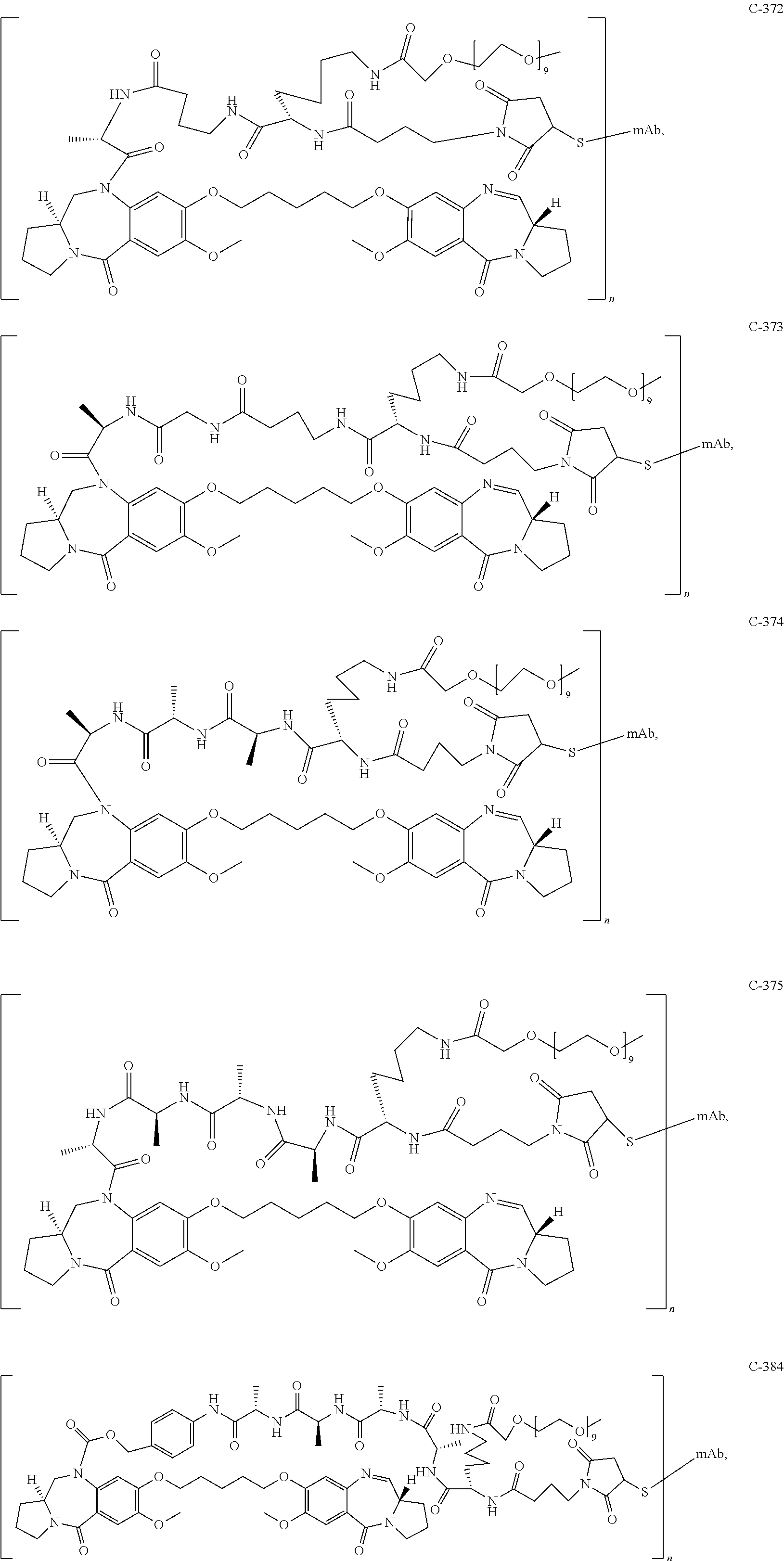

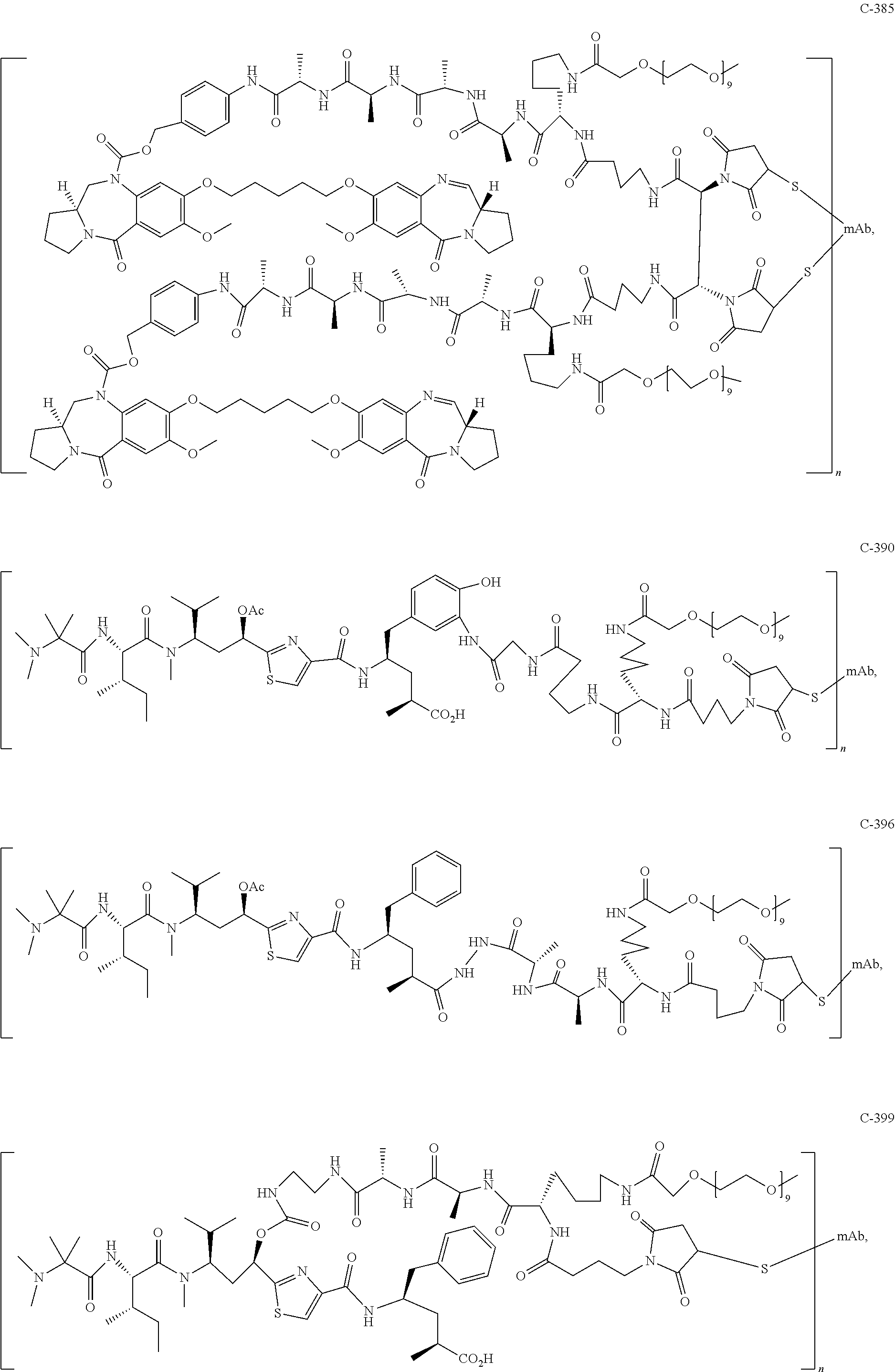

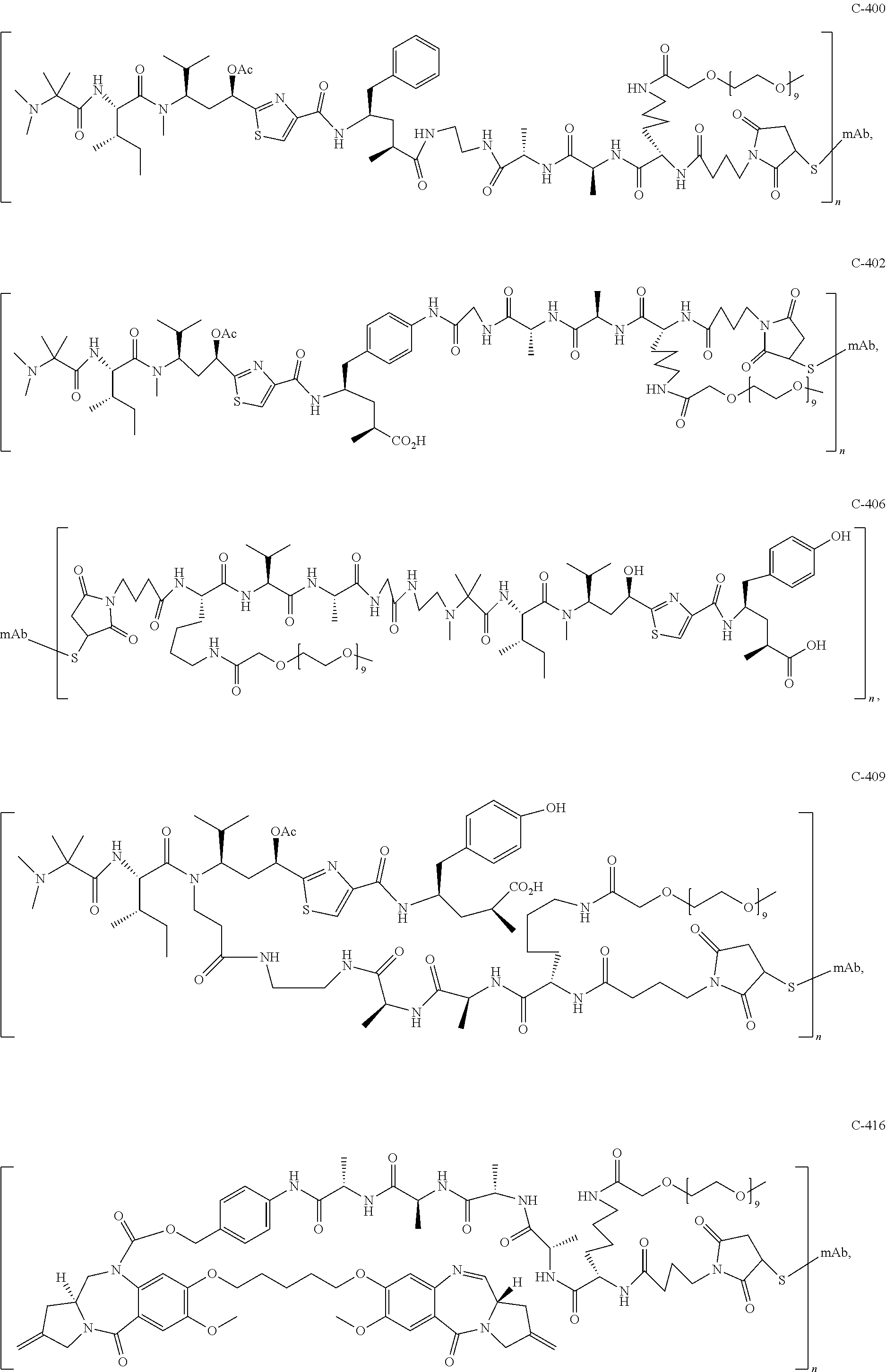

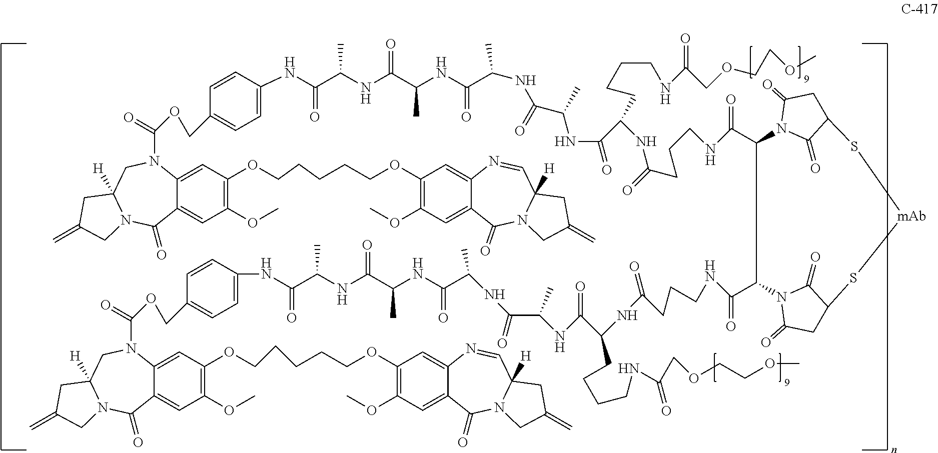

wherein mAb is the antibody of the invention, n=1˜20, preferably n=2˜8

### Example 379. DAR Analysis

DAR was analyzed by using HIC-HPLC, and the HPLC parameters are as follow Table 10:

TABLE 10
The condition for DAR analysis by HIC-HPLC.

| HPLC | Agilent 1260 | | | | | |
|---|---|---|---|---|---|---|
| Column | Thermo HIC butyl 4.6 × 100 mm | | | | | |
| Phase A | 0.5M (NH₄)₂SO₄ + 100 mM NaH₂PO₄, | | | | | |
| Phase B | 100 mM NaH₂PO₄, | | | | | |
| Sample | Dilute with buffer A to about 2 | | | | | |
| | mg/mL, injection volume 10 μL | | | | | |
| Rate | 0.8 mL/min | | | | | |
| Wavelength | 280 nm | | | | | |
| Column Temp. | 30° C. | | | | | |
| | Time (min) | 0 | 35 | 40 | 41 | 45 |
| Gradient | Phase A (%) | 80 | 0 | 0 | 80 | 80 |
| | Phase B (%) | 20 | 100 | 100 | 20 | 20 |

### Example 380. General Preparation of Formulation of the Conjugates

In a liquid formulation of 80 mg of each conjugate: C-25, C-30, C-36, C-45, C-51, C-58, C-68a, C-68b, C-68c, C-72a, C-72b, C-73, C-83, C-88, C-91, C-96, C-102, C-115, C-120, C-121, C-126, C-130, C-137, C-140, C-152, C-157, C-168, C-178, C-181a, C-181b, C-181c, C-181d, C-190, C-192, C-195, C-202, C-209, C-215, C-221, C-227, C-233, C-241, C-255, C-258, C-267, C-277, C-283, C-284, C-290, C-295, C-300, C-306, C-309, C-310, C-312, C-319, C-322, C-326, C-328, C-330, C-332, C-334, C-335, C-336, C-337, C-338, C-361, C-362, C-372, C-373, C-374, C-375, C-384, C-385, C-390, C-396, C-399, C-400, C-402, C-406, C-409, C-416, C-417,in the 10 mL of borosilicate vial containing 240 mg of sucrose, 0.8 mg of polysorbate-80, 24 mg of sodium citrate in 4 mL of sterile water were adjusted with citric acid to pH 6.0. Then each of the conjugate solution was lyophilized at temperature from −65° C. to 0° C., and to RT at reduced pressure (5˜10 torr) to form a dryness cake. The cake conjugates were stored at 2˜8° C., and then reconstituted with 4 mL of sterile water for further application.

### Example 381

In vitro cytotoxicity evaluation of the BCMA-conjugate: C-25, C-30, C-36, C-45, C-51, C-58, C-68a, C-68b, C-68c, C-72a, C-72b, C-73, C-83, C-88, C-91, C-96, C-102, C-11S, C-120, C-121, C-126, C-130, C-137, C-140, C-152, C-157, C-168, C-178, C-181a, C-181b, C-181c, C-181d, C-190, C-192, C-195, C-202, C-209, C-215, C-221, C-227, C-233, C-241, C-255, C-258, C-267, C-277, C-283, C-284, C-290, C-295, C-300, C-306, C-309, C-310, C-312, C-319, C-322, C-326, C-328, C-330, C-332, C-334, C-335, C-336, C-337, C-338, C-361, C-362, C-372, C-373, C-374, C-375, C-384, C-385, C-390, C-396, C-399, C-400, C-402, C-406, C-409, C-416, C-417, in comparison with Paclitaxel and Belantamabmc-MMAF conjugate prepared in house with the traditional conjugation process (Doronina S, O., et al, Bioconjug Chem. 2006, 17(1):114-24).

The cell lines used in the cytotoxicity assays were (1). NCI-H929, JJN3, U266, and MM1S were obtained from ATCC, and 8226-2A1cell is Myeloma antigen express cells through culturing and clone-picking of ATCC's RPMI-8226. The cells were grown according to the provider manuals. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/mL) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100. The MTT results of BCMA-ADCs are listed in Table 7.

TABLE 7
MTT assays of the BCMA antibody conjugates against tumor cells
of NCI-H929, MM1S, JJN-3, at 6000 cells, 96 h incubation. The
DAR indicated in the table 7 were the conjugate DARs prepared
via homogeneous conjugation process of the invention:

| | DAR | IC50 (n [symbol] | IC50 (n [symbol] | IC50 (n [symbol] |
|---|---|---|---|---|
| ADC Compound | ratio | NCI-H929 | MM1S | JJN-3 |
| C-25 | 4.2 | 6.3 | 6.5 | 7.9 |
| C-30 | 4.4 | 3.22 | 3.13 | 4.71 |
| C-36 | 4.4 | 3.13 | 3.21 | 4.63 |
| C-45 | 4.4 | 1.15 | 1.08 | 1.77 |
| C-51 | 4.4 | 1.05 | 1.32 | 1.98 |
| C-58a | 4.6 | 0.78 | 0.95 | 1.56 |
| C-68a | 4.6 | 0.36 | 0.39 | 1.17 |
| C-68b | 4.4 | 0.37 | 0.39 | 1.28 |
| C-68c | 4.6 | 0.29 | 0.41 | 1.39 |
| C-72a | 4.4 | 0.43 | 0.52 | 0.98 |
| C-72b | 4.4 | 0.59 | 0.61 | 1.12 |
| C-73 | 4.2 | 0.68 | 0.71 | 1.35 |
| C-83 | 4.2 | 0.02 | 0.04 | 0.08 |
| C-88 | 4.2 | 0.08 | 0.11 | 0.19 |
| C-91 | 4.2 | 0.07 | 0.13 | 0.14 |
| C-96 | 4.2 | 0.13 | 0.16 | 0.21 |
| C-102 | 4.0 | 0.89 | 0.14 | 0.28 |
| C-115 | 4.0 | 0.02 | 0.04 | 0.10 |
| C-120 | 4.2 | 0.21 | 0.23 | 0.31 |
| C-121 | 4.2 | 0.06 | 0.09 | 0.12 |
| C-126 | 4.2 | 0.18 | 0.21 | 0.29 |
| C-130 | 4.3 | 0.87 | 0.98 | 1.17 |
| C-137 | 4.4 | 0.75 | 0.87 | 0.91 |
| C-140 | 4.2 | 0.77 | 0.83 | 0.96 |
| C-152 | 4.2 | 0.78 | 0.87 | 0.99 |
| C-157 | 4.6 | 0.70 | 0.87 | 0.95 |
| C-168 | 4.2 | 0.59 | 0.89 | 1.28 |
| C-178 | 4.1 | 0.38 | 0.46 | 0.65 |
| C-181a | 4.2 | 0.92 | 0.98 | 1.31 |
| C-181b | 4.2 | 0.95 | 0.93 | 1.19 |
| C-181c | 4.4 | 0.65 | 0.80 | 1.02 |
| C-181d | 4.4 | 0.49 | 0.72 | 0.89 |
| C-190 | 4.8 | 1.18 | 1.11 | 1.28 |
| C-192 | 4.6 | 1.07 | 0.98 | 1.21 |
| C-195 | 4.6 | 1.12 | 1.38 | 1.69 |
| C-209 | 3.4 | 1.48 | 1.57 | 1.67 |
| C-215 | 4.6 | 0.80 | 0.96 | 1.31 |
| C-221 | 4.2 | 0.04 | 0.05 | 0.08 |
| C-227 | 4.4 | 0.05 | 0.06 | 0.08 |
| C-233 | 4.4 | 0.05 | 0.06 | 0.11 |
| C-241 | 4.5 | 0.13 | 0.29 | 0.31 |
| C-255 | 4.4 | 0.06 | 0.08 | 0.11 |
| C-258 | 4.4 | 0.05 | 0.08 | 0.09 |
| C-267 | 4.4 | 0.12 | 0.18 | 0.38 |
| C-277 | 4.2 | 0.03 | 0.06 | 0.09 |
| C-283 | 4.1 | 1.22 | 1.60 | 1.96 |
| C-284 | 4.0 | 0.04 | 0.07 | 0.12 |
| C-290 | 4.1 | 0.35 | 0.49 | 0.93 |
| C-295 | 4.2 | 1.01 | 1.06 | 1.03 |
| C-300 | 4.4 | 0.29 | 0.25 | 0.39 |
| C-306 | 4.4 | 0.08 | 0.09 | 0.12 |
| C-309 | 4.5 | 0.06 | 0.09 | 0.17 |
| C-310 | 4.4 | 0.15 | 0.19 | 0.33 |
| C-312 | 4.5 | 0.13 | 0.15 | 0.19 |
| C-319 | 4.4 | 0.25 | 0.27 | 0.38 |
| C-322 | 4.4 | 0.29 | 0.34 | 0.59 |
| C-326 | 4.4 | 0.45 | 0.59 | 0.73 |
| C-328 | 4.4 | 0.06 | 0.09 | 0.19 |
| C-330 | 4.4 | 0.21 | 0.29 | 0.92 |
| C-332 | 4.4 | 0.08 | 0.19 | 0.31 |
| C-334 | 4.1 | 0.20 | 0.13 | 0.62 |
| C-335 | 4.0 | 0.23 | 0.32 | 0.83 |
| C-336 | 4.2 | 0.13 | 0.16 | 0.39 |
| C-337 | 4.0 | 0.57 | 0.49 | 0.97 |
| C-338 | 4.1 | 0.51 | 0.50 | 0.61 |
| C-361 | 4.0 | 5.93 | 7.50 | 8.57 |
| C-362 | 4.2 | 5.63 | 8.54 | 6.67 |
| C-373 | 4.1 | 4.47 | 5.57 | 5.78 |
| C-374 | 4.1 | 5.46 | 5.62 | 5.66 |
| C-375 | 4.2 | 5.32 | 5.49 | 4.79 |
| C-384 | 4.0 | 1.41 | 1.63 | 1.87 |
| C-385 | 4.2 | 1.30 | 1.49 | 1.63 |
| C-390 | 4.4 | 0.06 | 0.05 | 0.09 |
| C-396 | 4.4 | 0.04 | 0.06 | 0.07 |
| C-399 | 4.4 | 0.17 | 0.28 | 0.39 |
| C-400 | 4.1 | 0.07 | 0.04 | 0.06 |
| C-402 | 4.2 | 0.03 | 0.04 | 0.06 |
| C-406 | 4.4 | 0.11 | 0.14 | 0.29 |
| C-409 | 4.4 | 0.07 | 0.12 | 0.11 |
| C-416 | 4.0 | 0.08 | 0.09 | 0.11 |
| C-417 | 4.4 | 0.07 | 0.09 | 0.09 |
| Belantamabmc- | 4.0 | 0.08 | 0.11 | 0.41 |
| MMAF | | | | |
| Paclitaxel | | 2.89 | 3.28 | 4.31 |

[symbol]

indicates data missing or illegible when filed

### Example 382. Antitumor Activity In Vivo (BALB/c Nude Mice Bearing NCI-H929, or MM1S, Xenograft Tumors Independently)

The in vivo efficacy of BCMA conjugates of C-68a, C-83, C-115, C-126, C-137, C-181b, C-192, C-195, C-202, C-212b, C-258, C-277, C-290, C-306, C-385, C-390, C-396, C-399, C-400, C-402, C-406, and C-417along with Belantamabmc-MMAF against human multiple myeloma NCI-H929 cells or JJN-3 cells, in xenograft models. Five-week-old female BALB/c Nude mice (6 animals per group) were inoculated subcutaneously in the area under the right shoulder with respective carcinoma cells (5×10⁶ cells/mouse) in 0.1-0.2 mL of serum-free medium. The tumors were grown for 6-8 days to an average size of 150 mm³, or 9-10 days to an average size of 200 mm³. The animals were then randomly divided into different groups (6 animals per group). The first group of mice served as the control group and was treated with the phosphate-buffered saline (PBS) vehicle. The other groups were treated with conjugates at dose of 2 or 3 mg/Kg for NCI-H929 cell model or 5 mg/Kg for JJN-3 cell model, administered intravenously. Three dimensions of the tumor were measured every 3 or 4 days (twice a week) and the tumor volumes were calculated using the formula tumor volume=½(length×width×height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

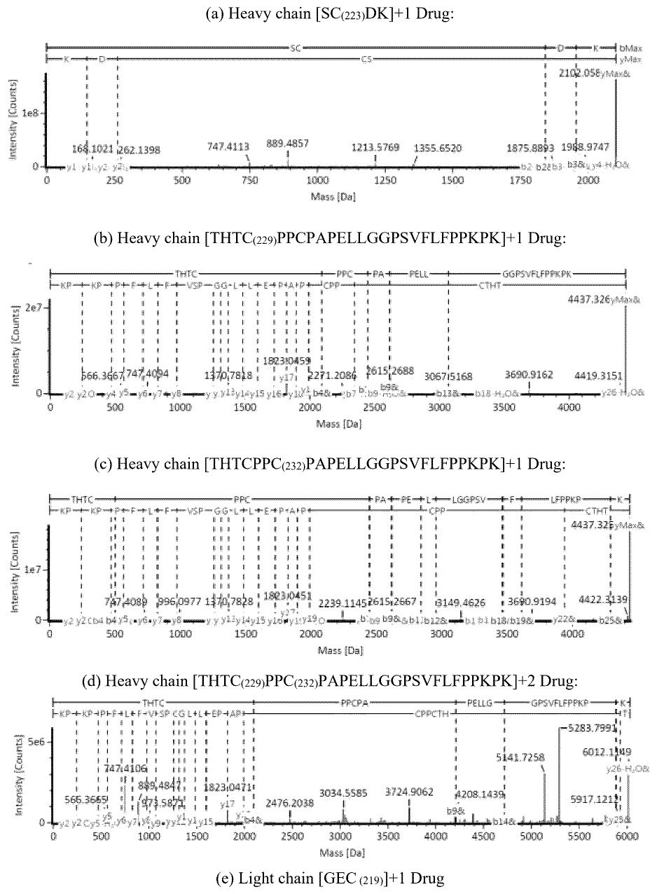

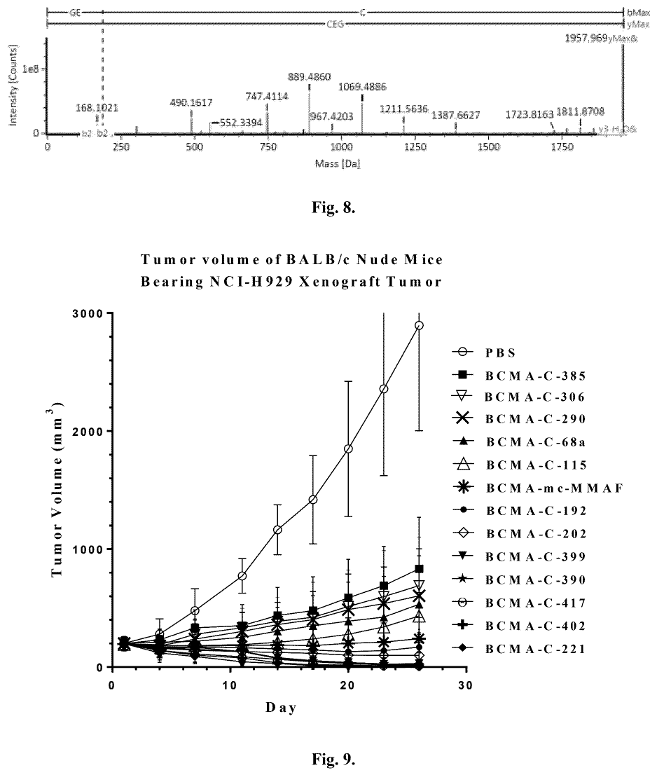

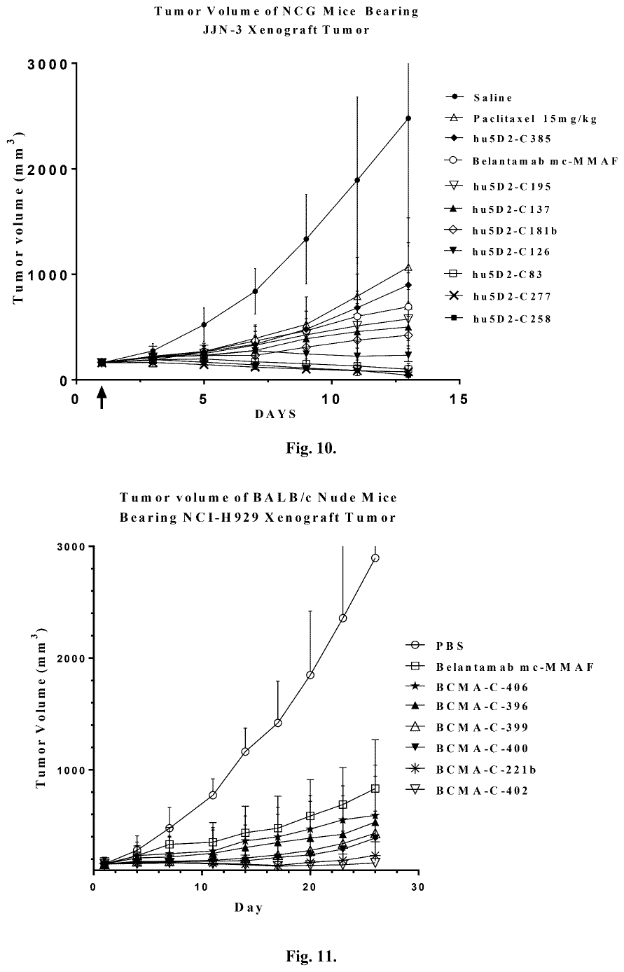

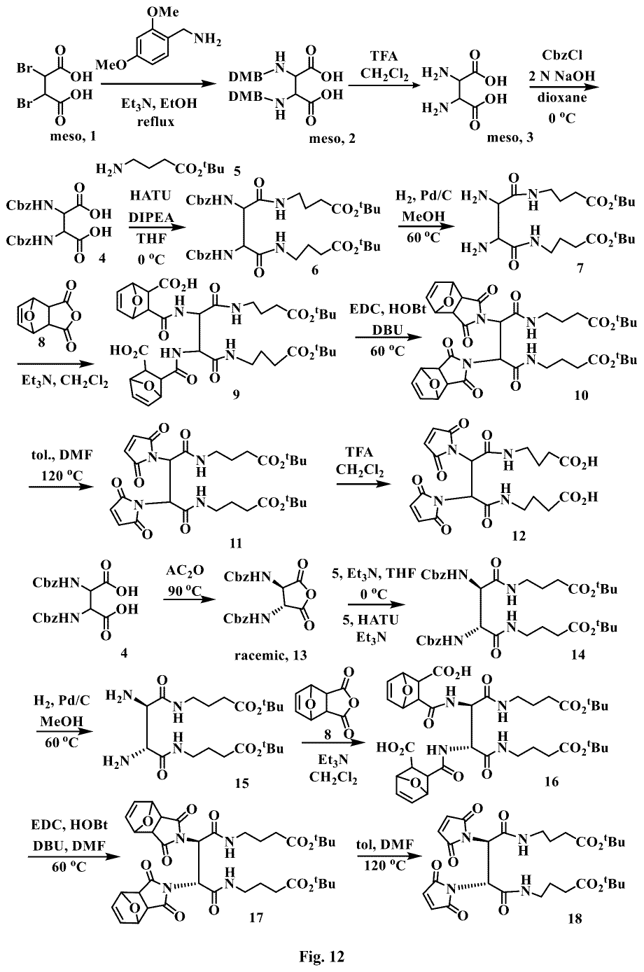

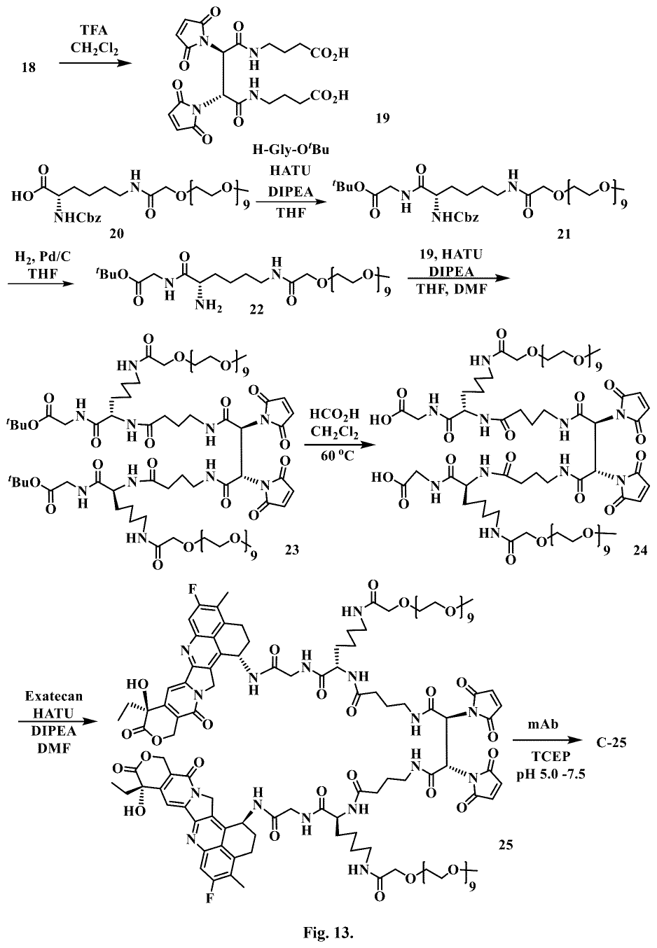

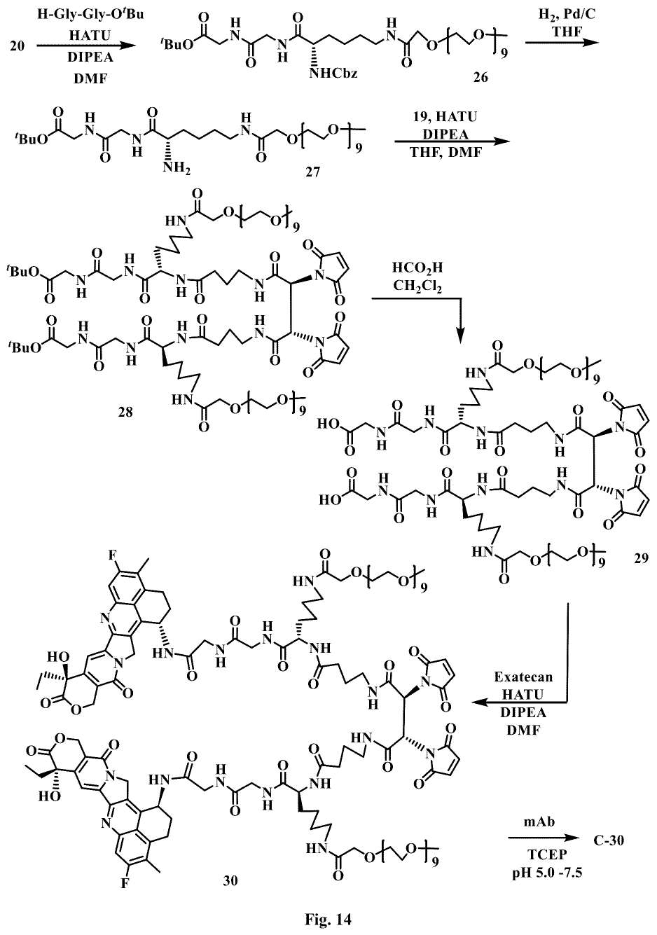

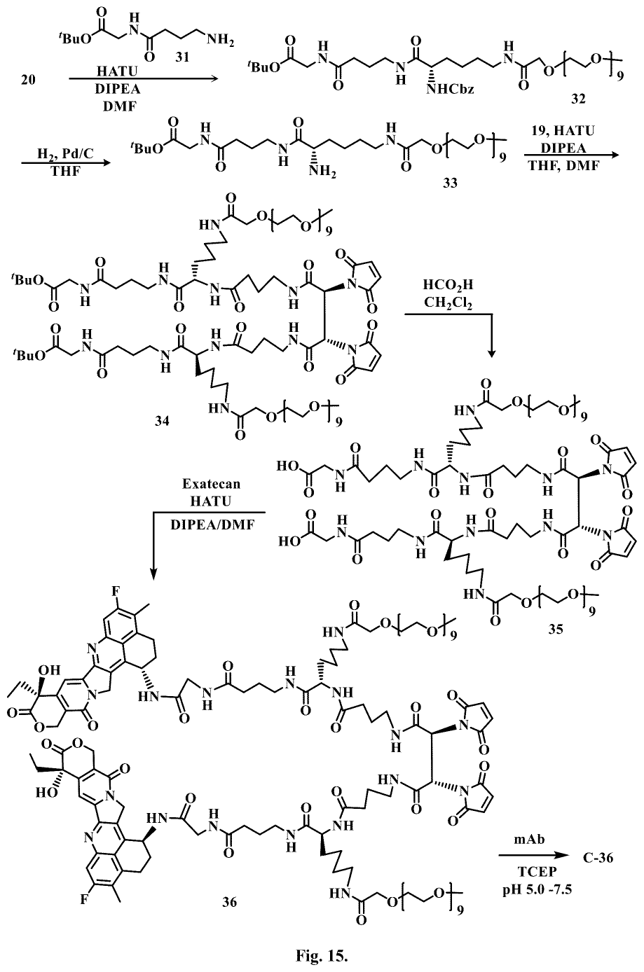

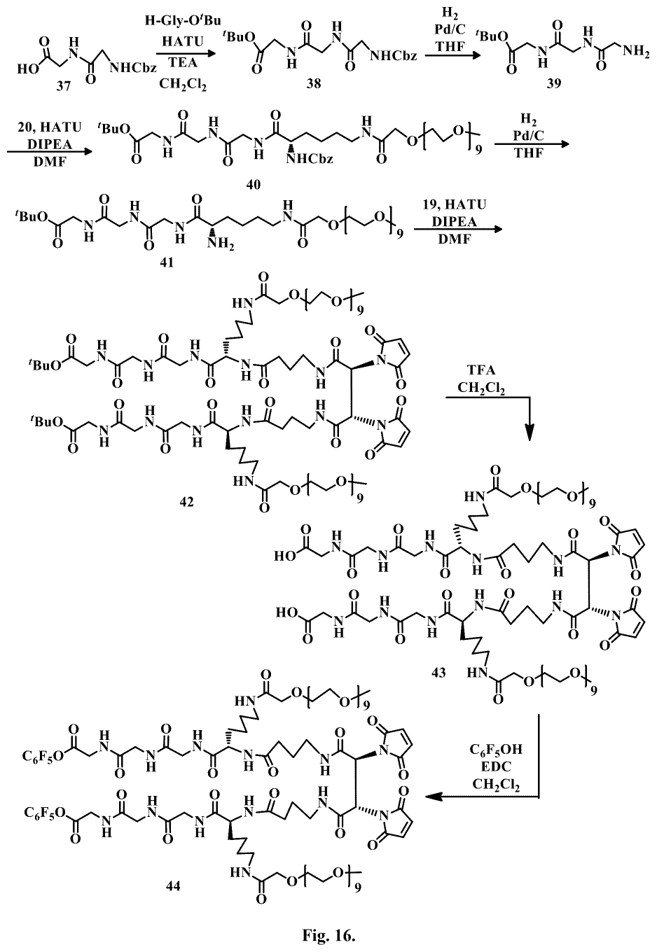

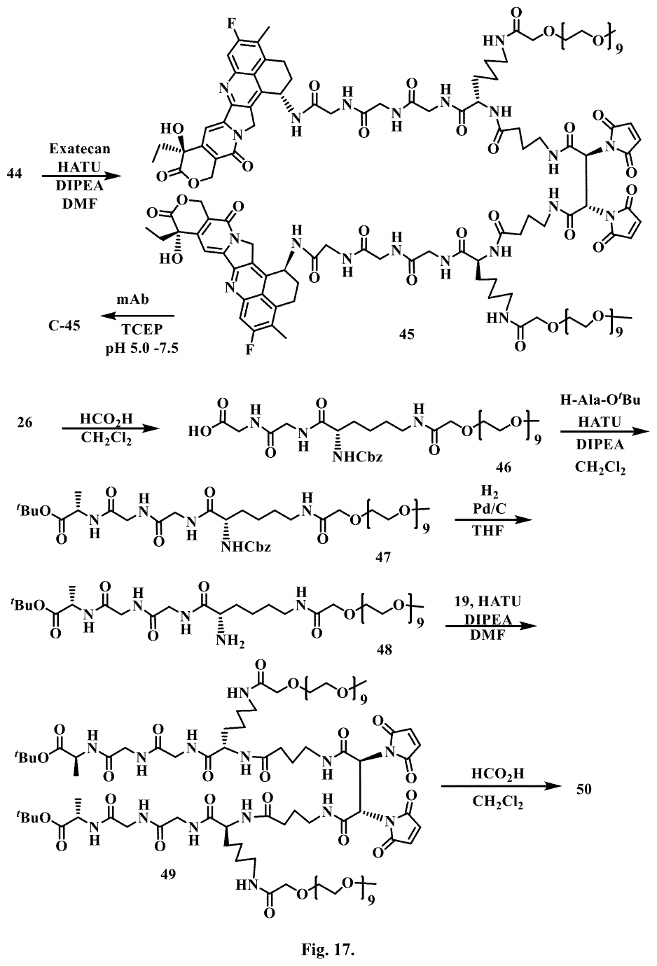

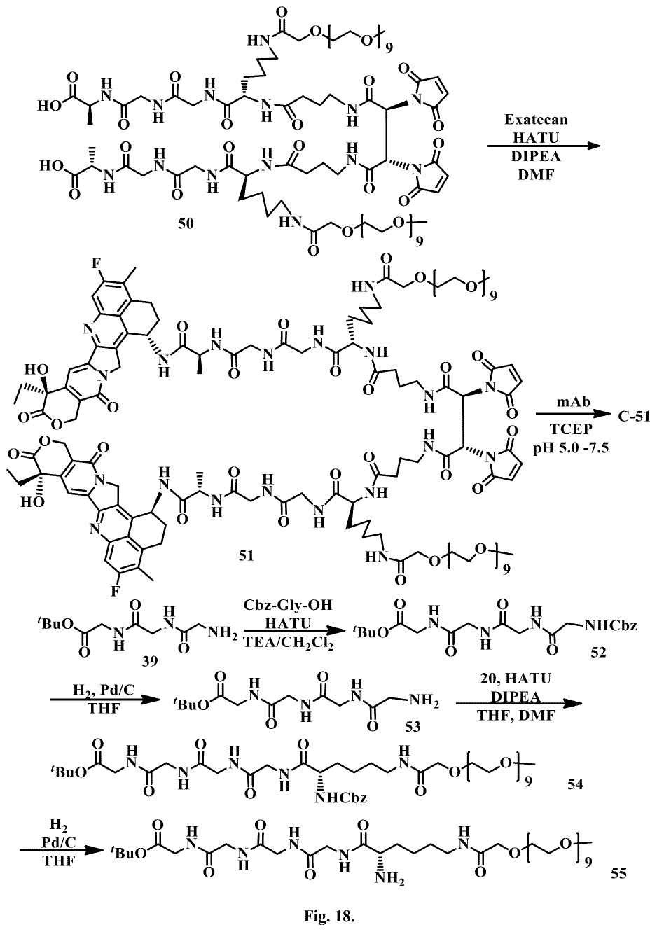

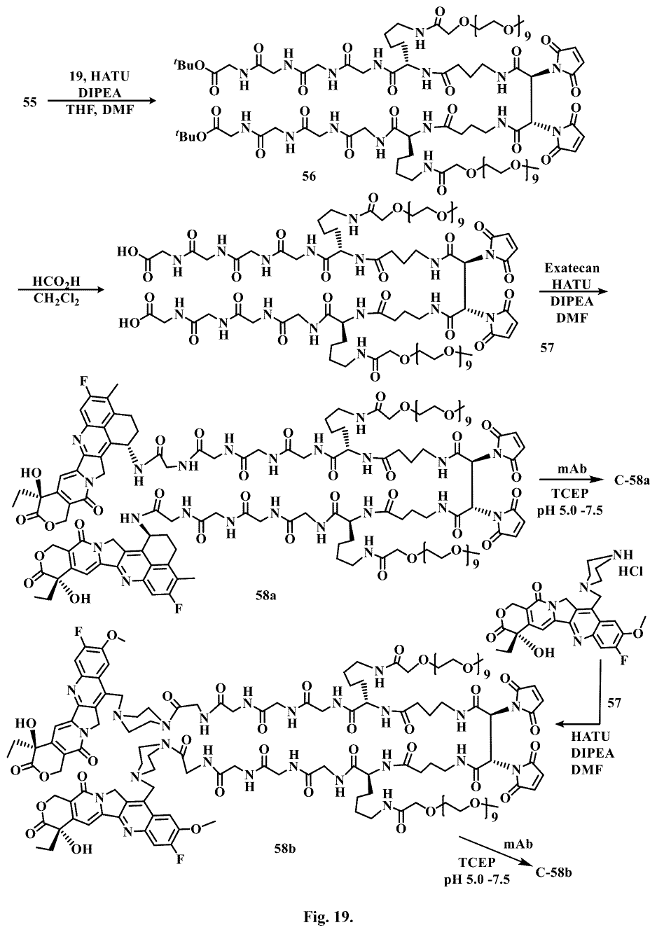

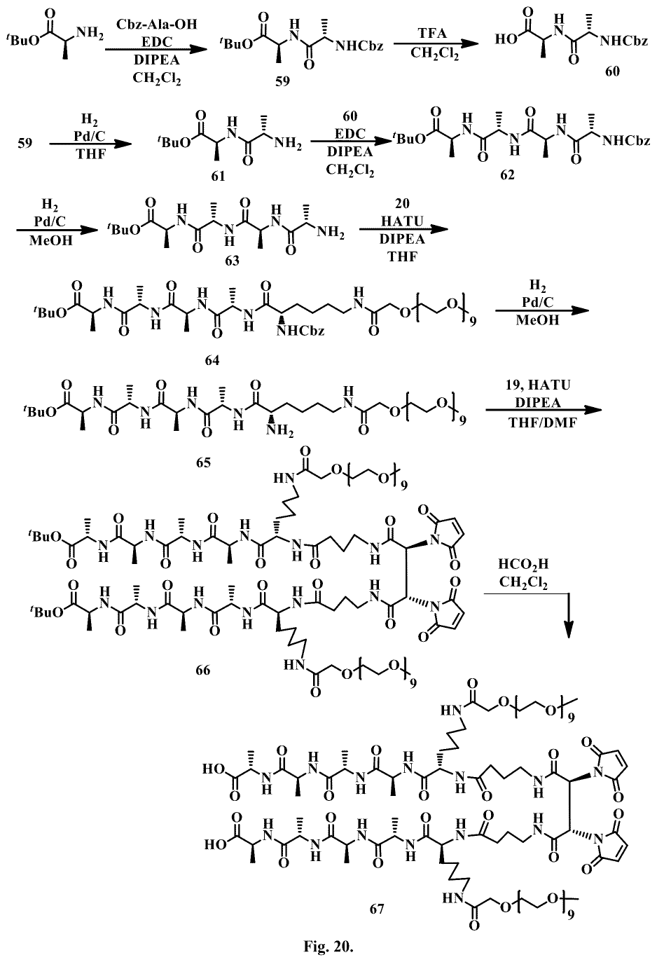

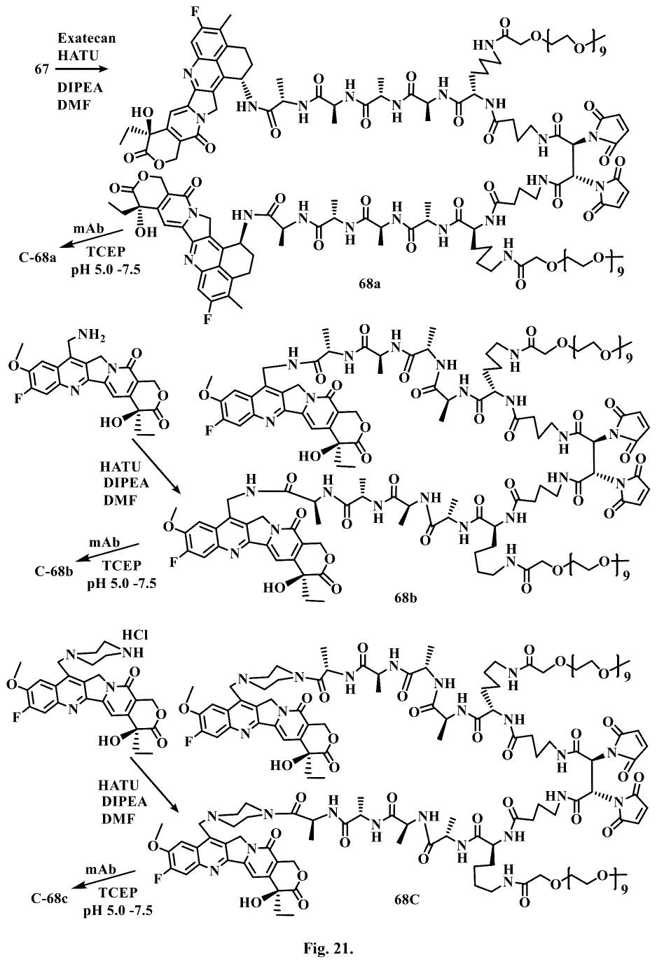

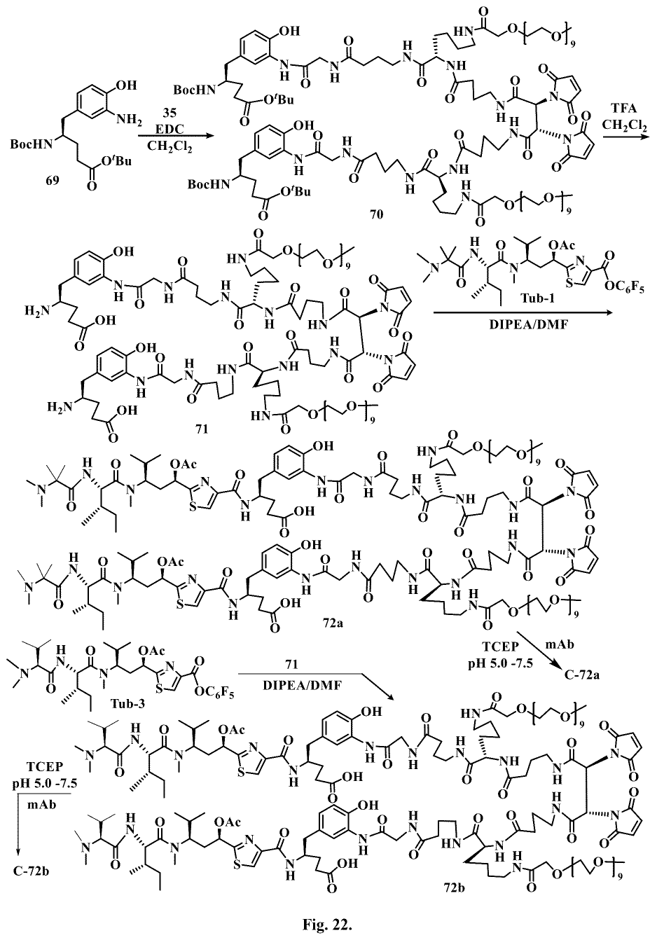

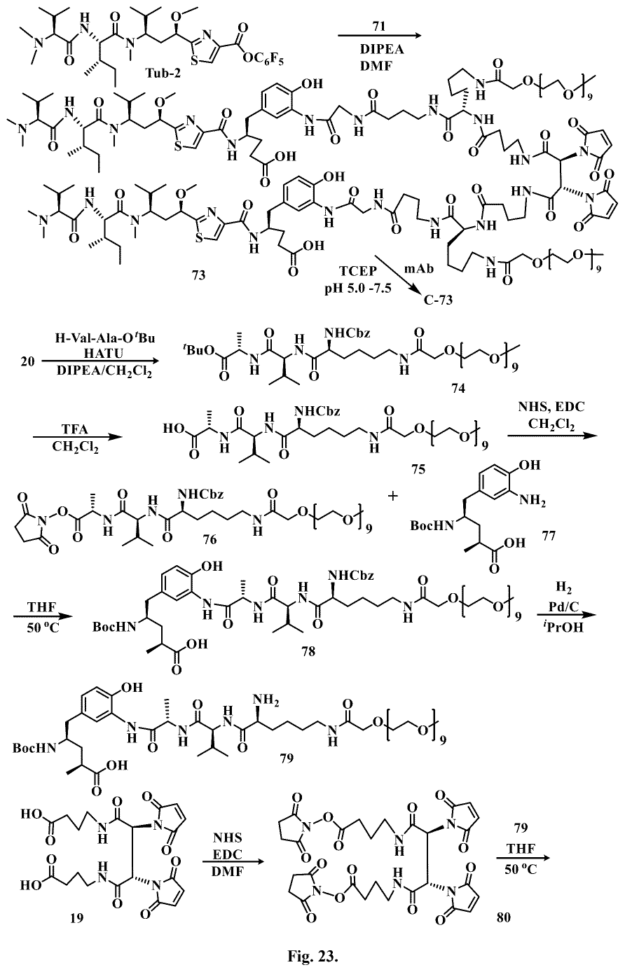

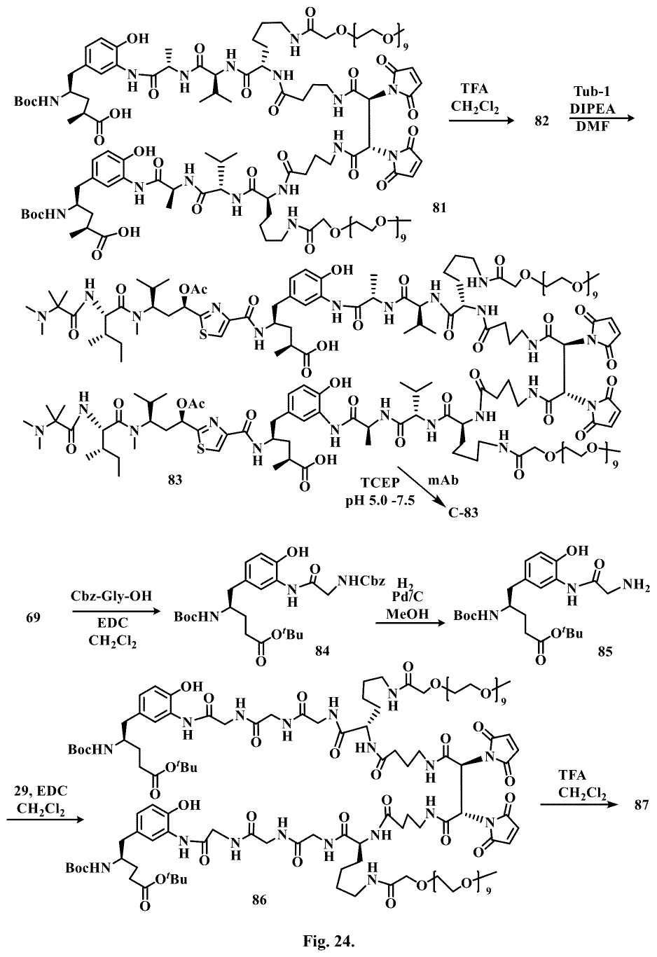

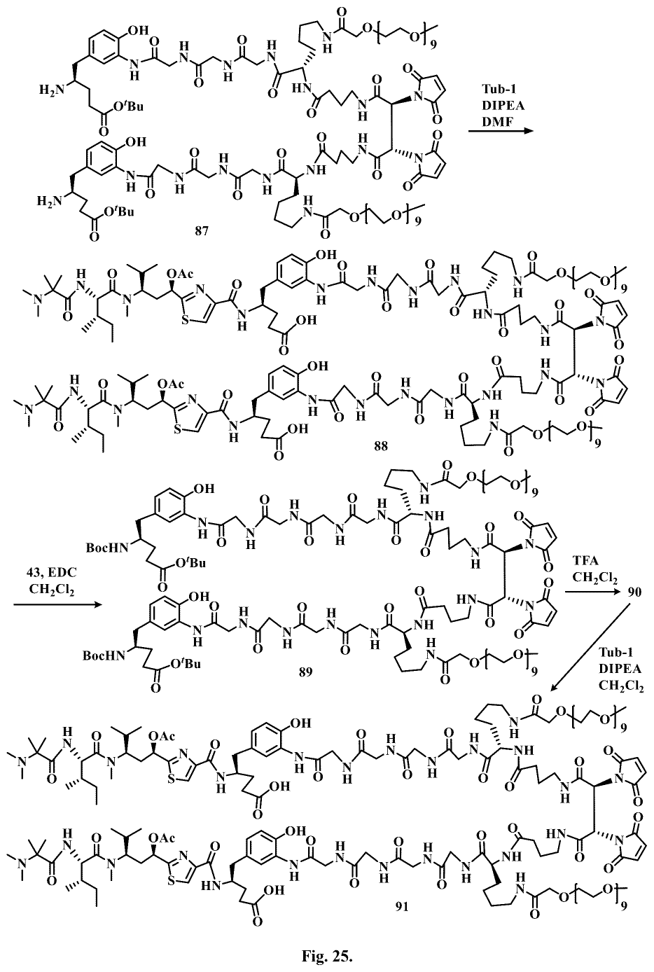

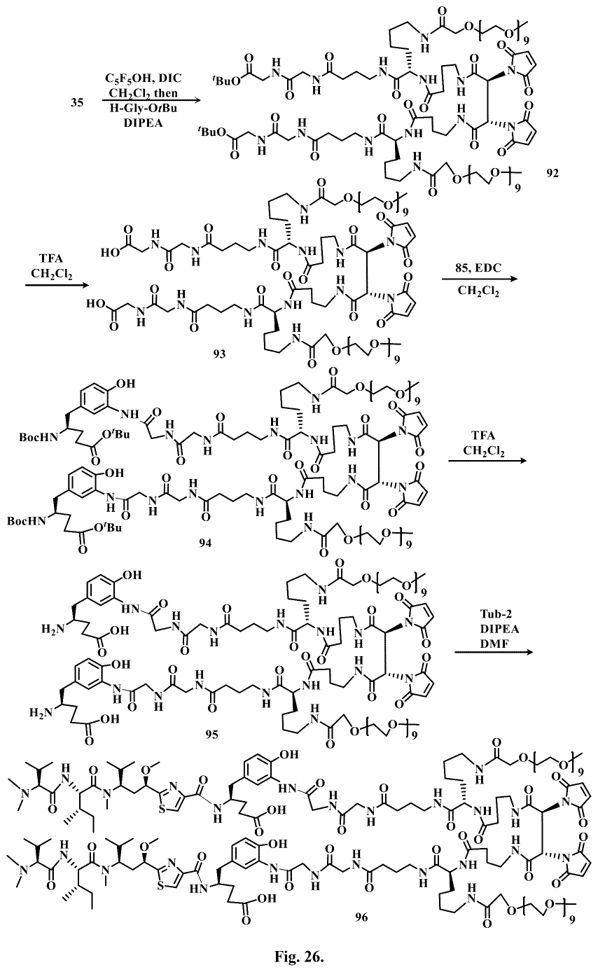

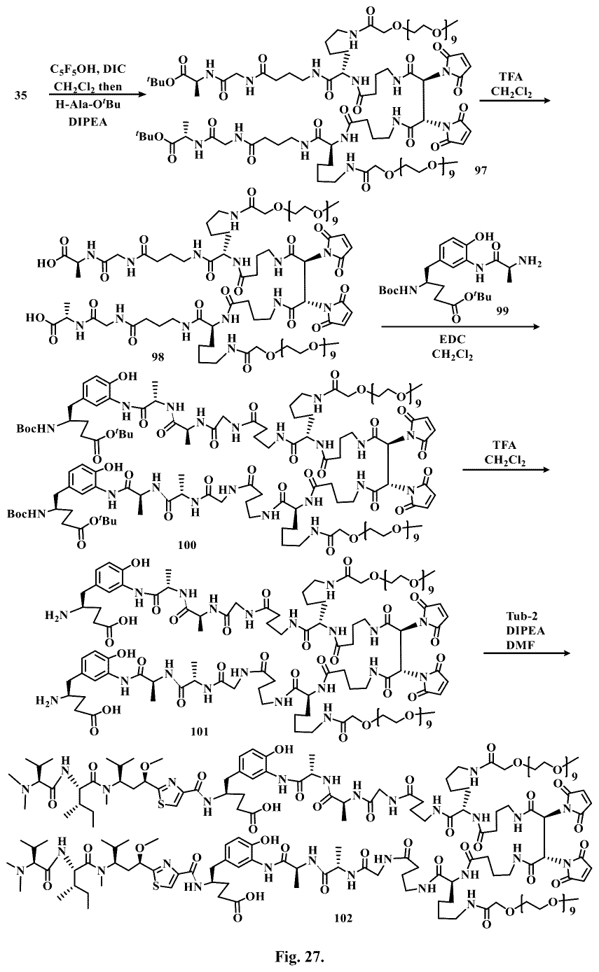

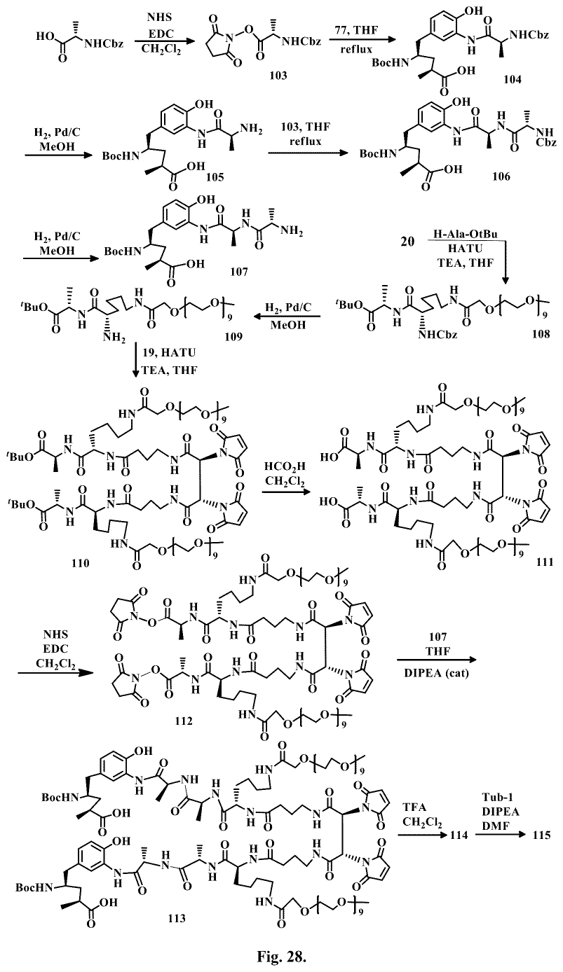

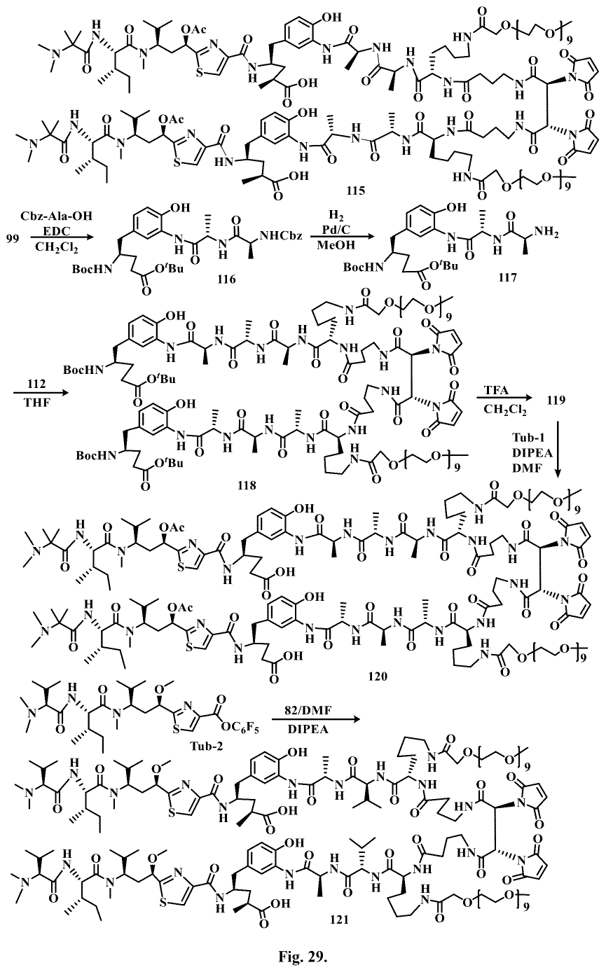

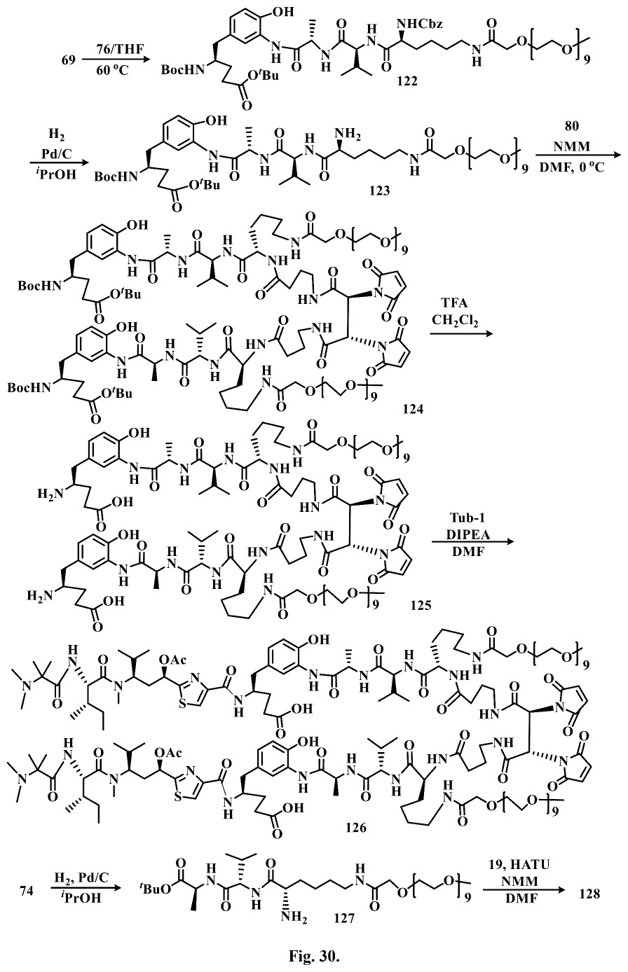

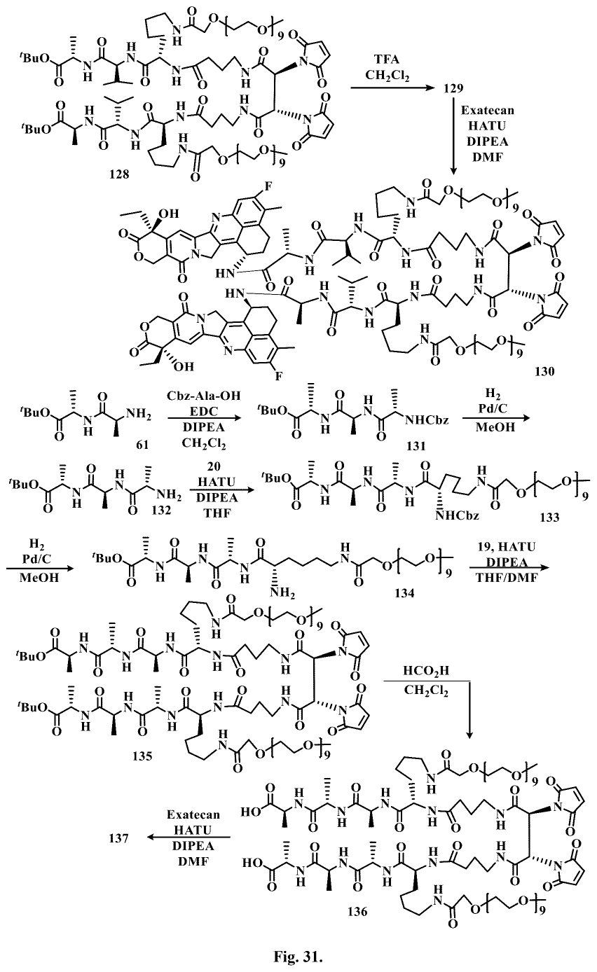

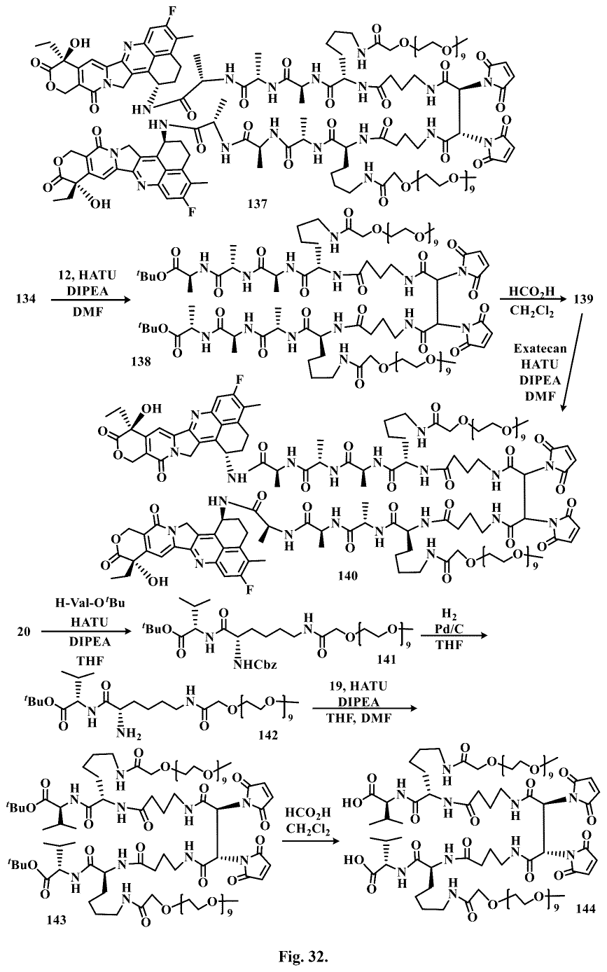

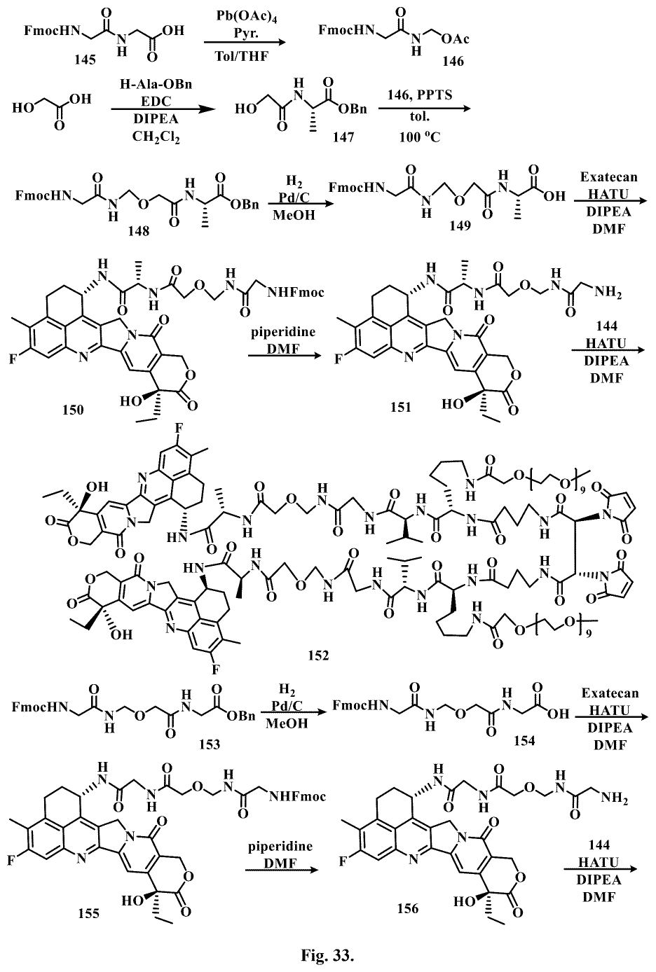

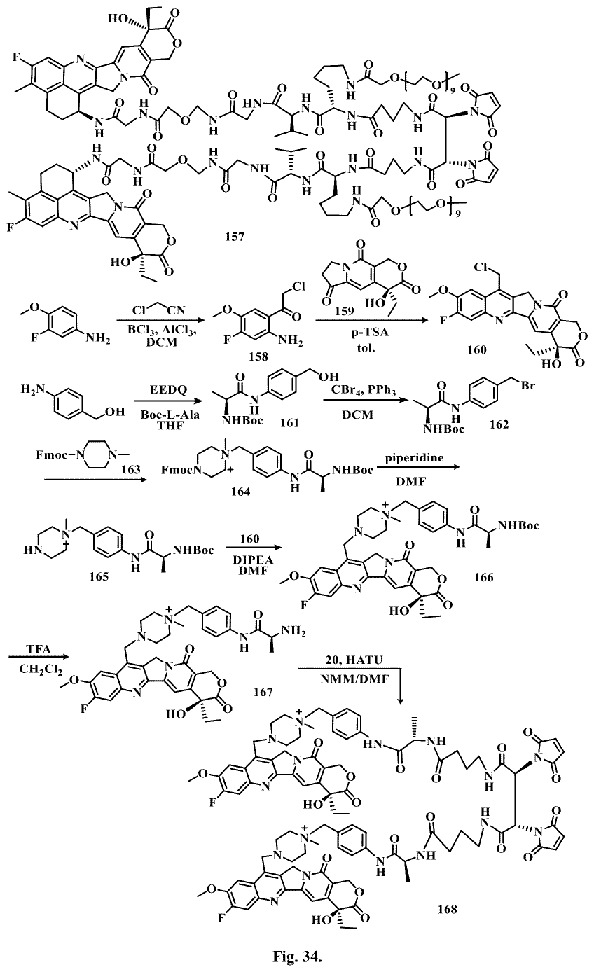

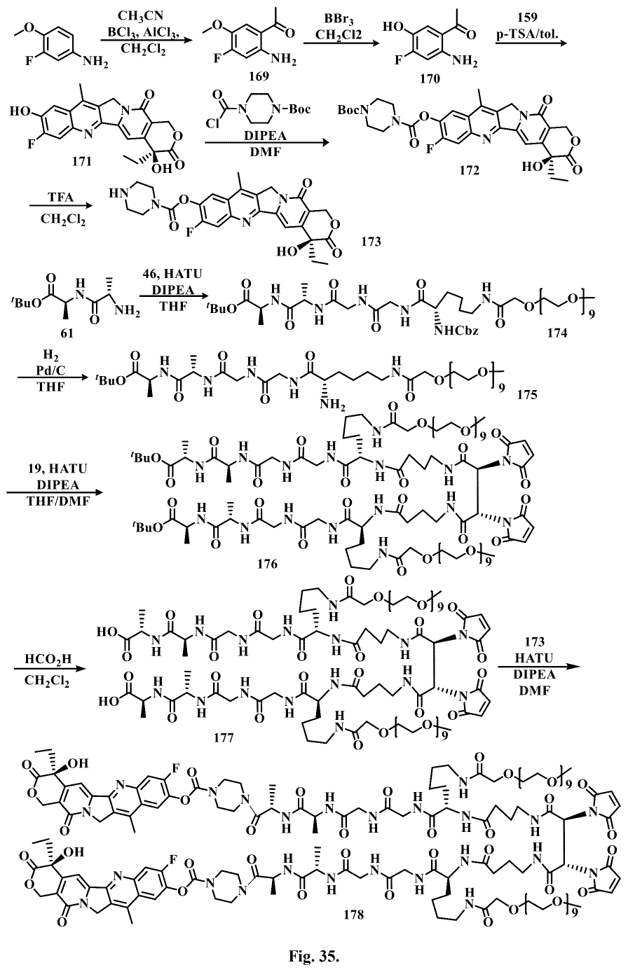

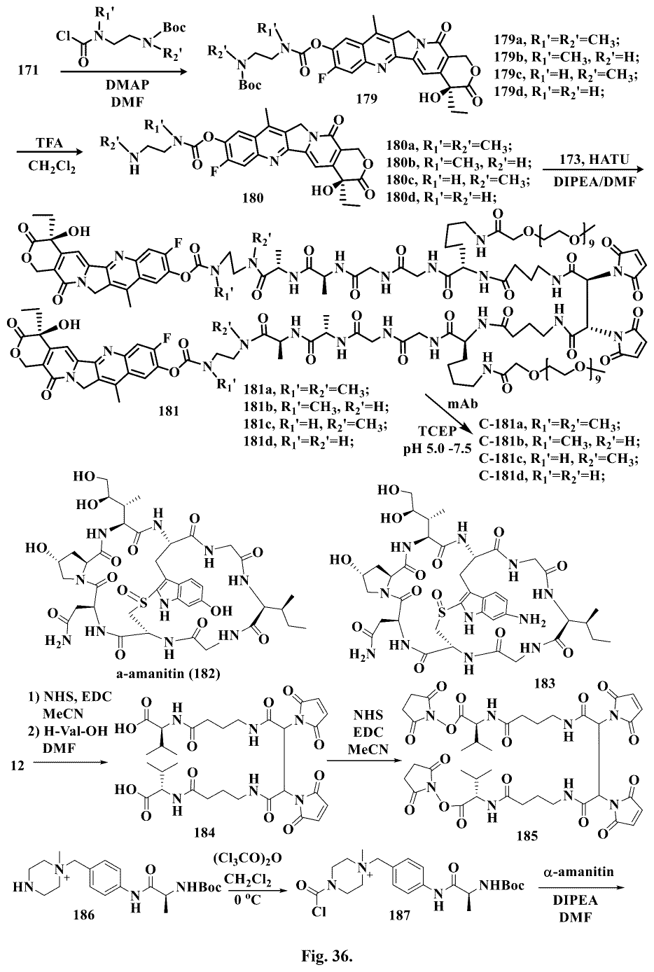

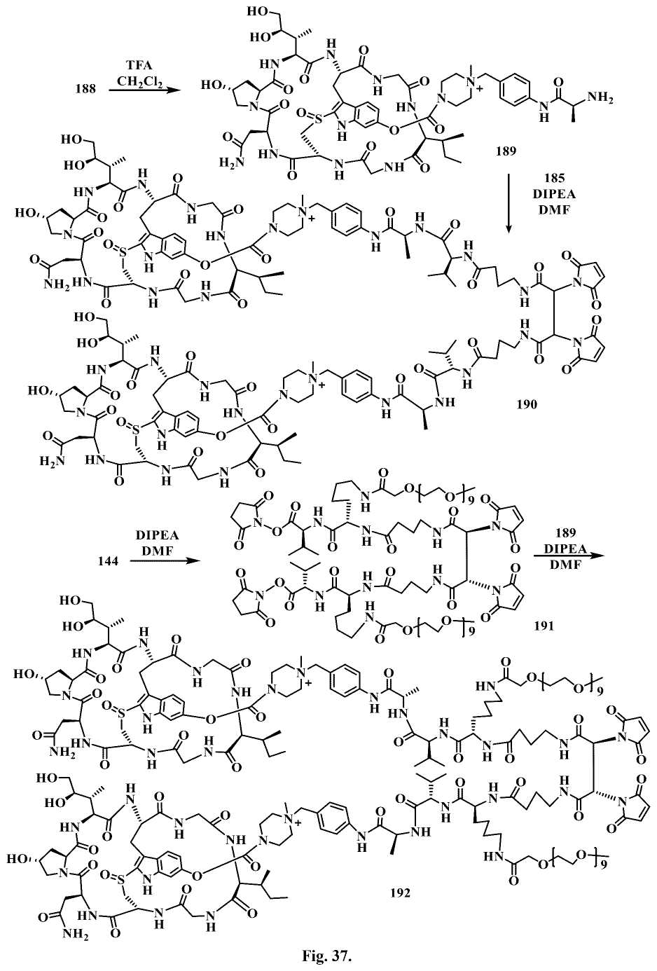

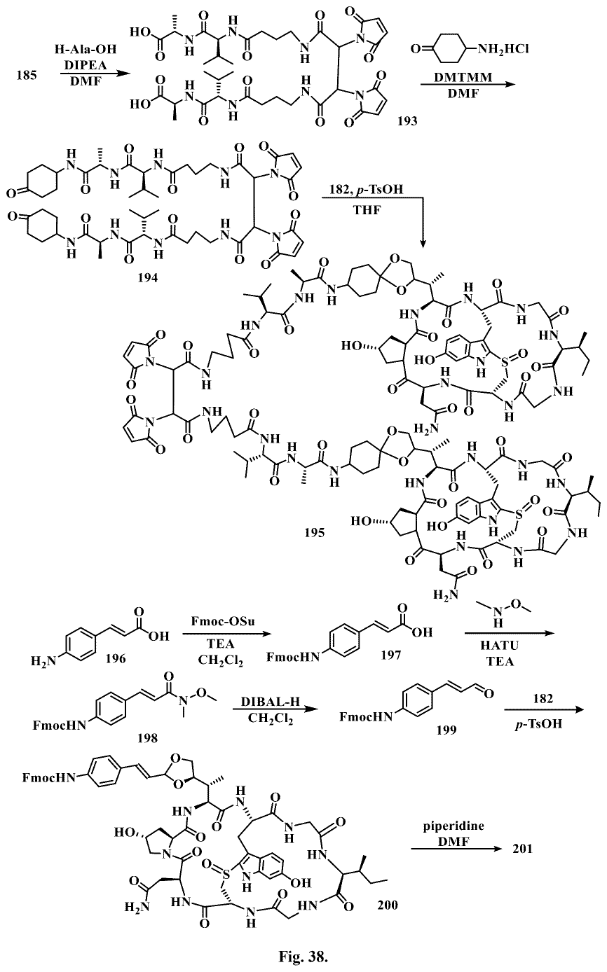

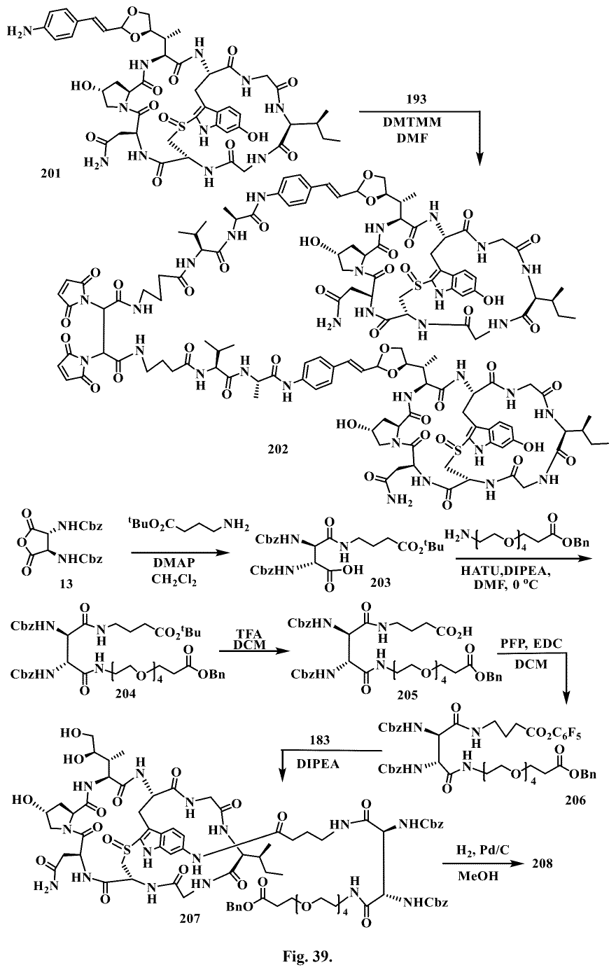

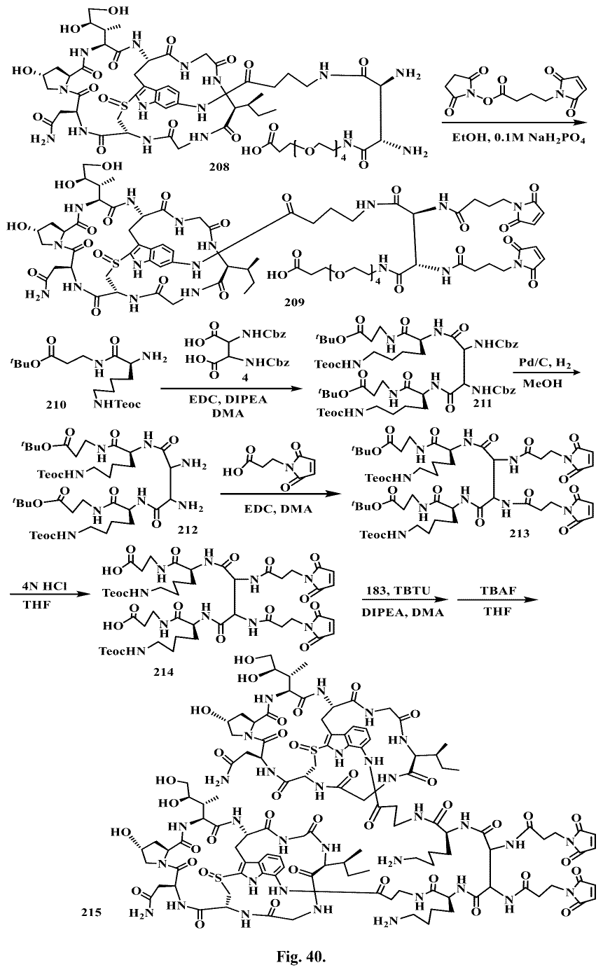

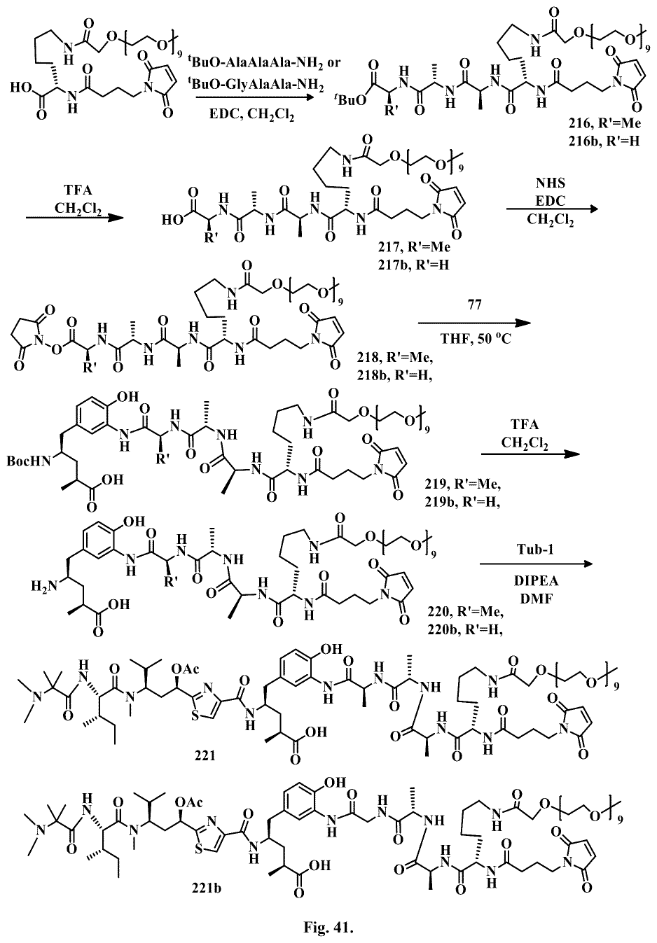

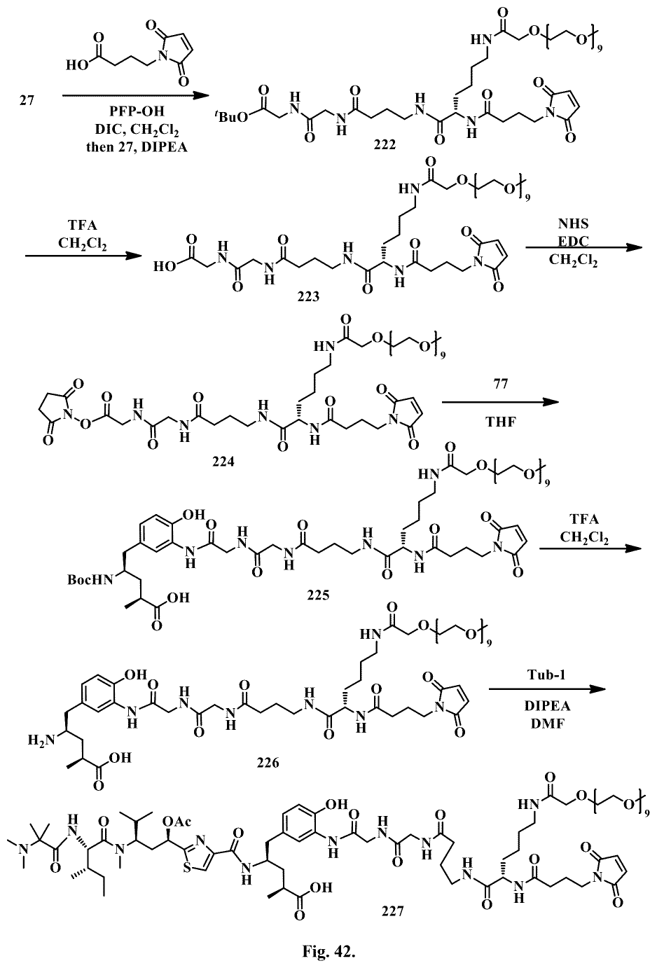

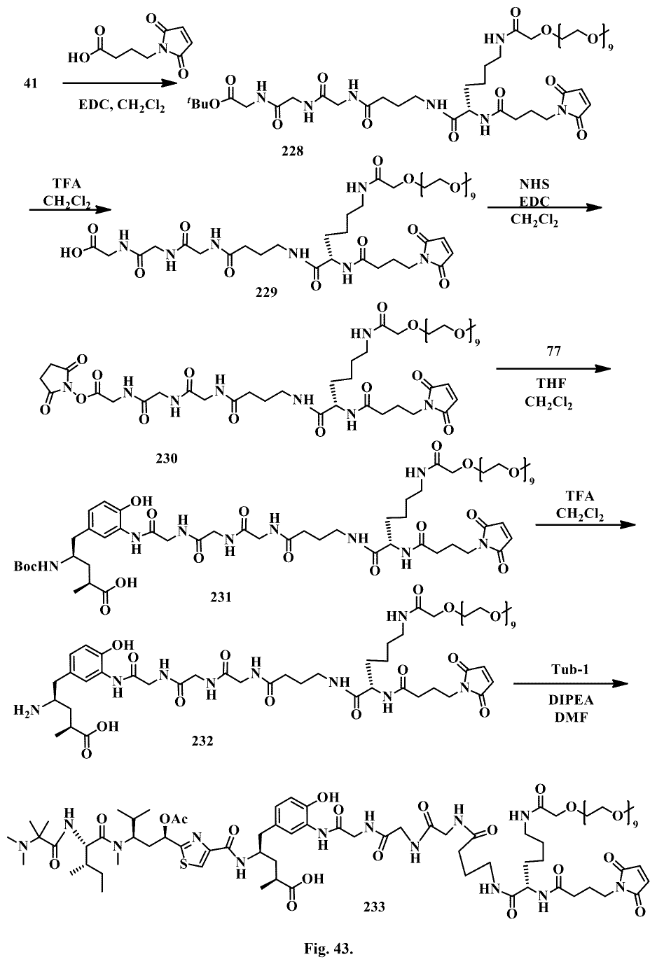

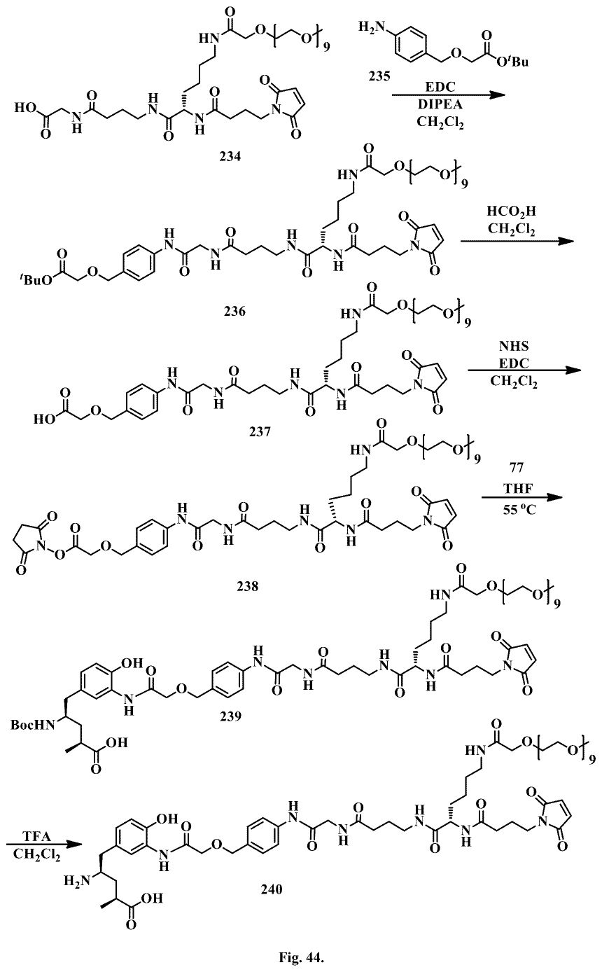

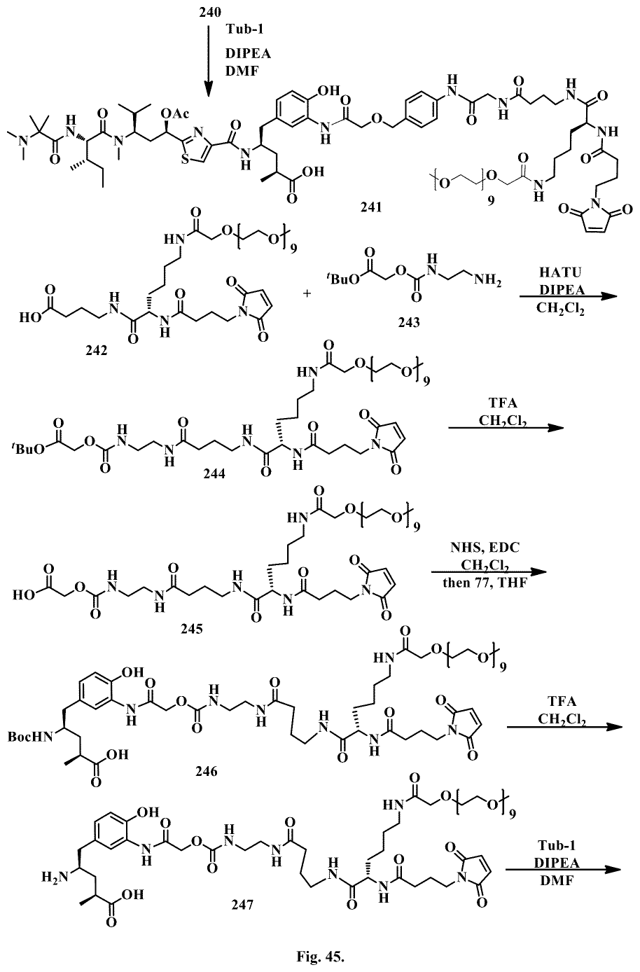

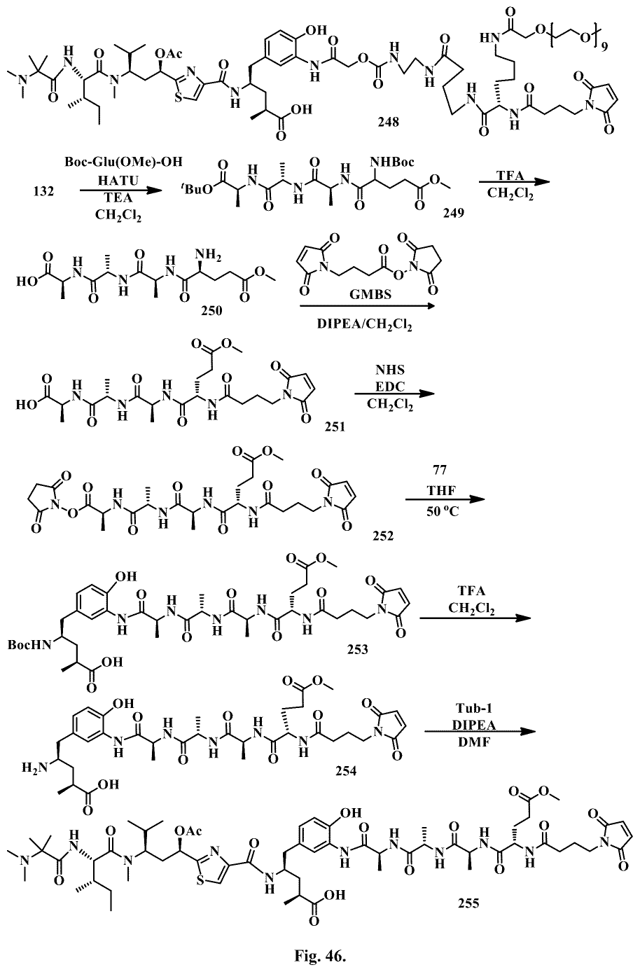

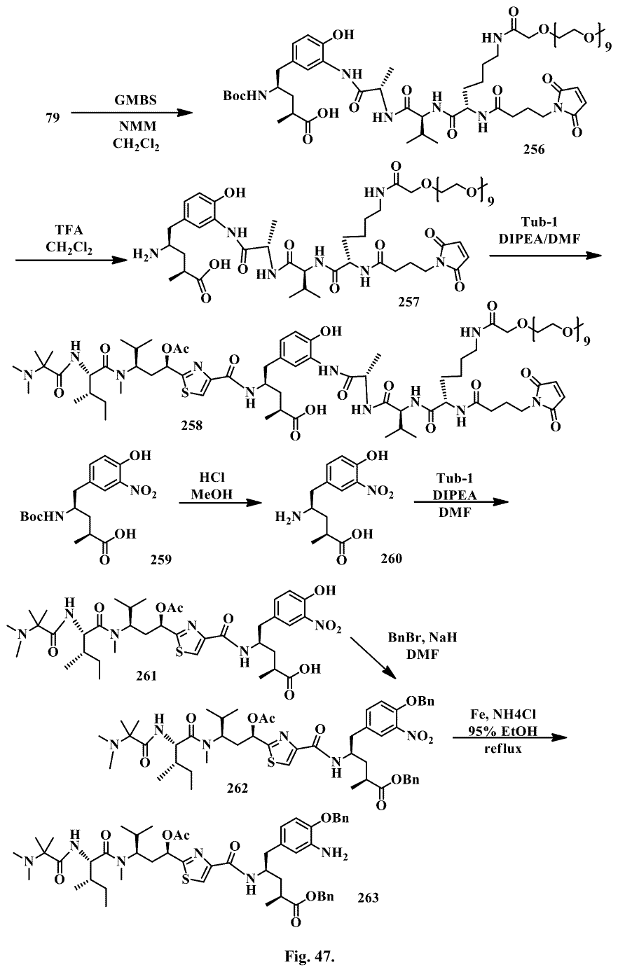

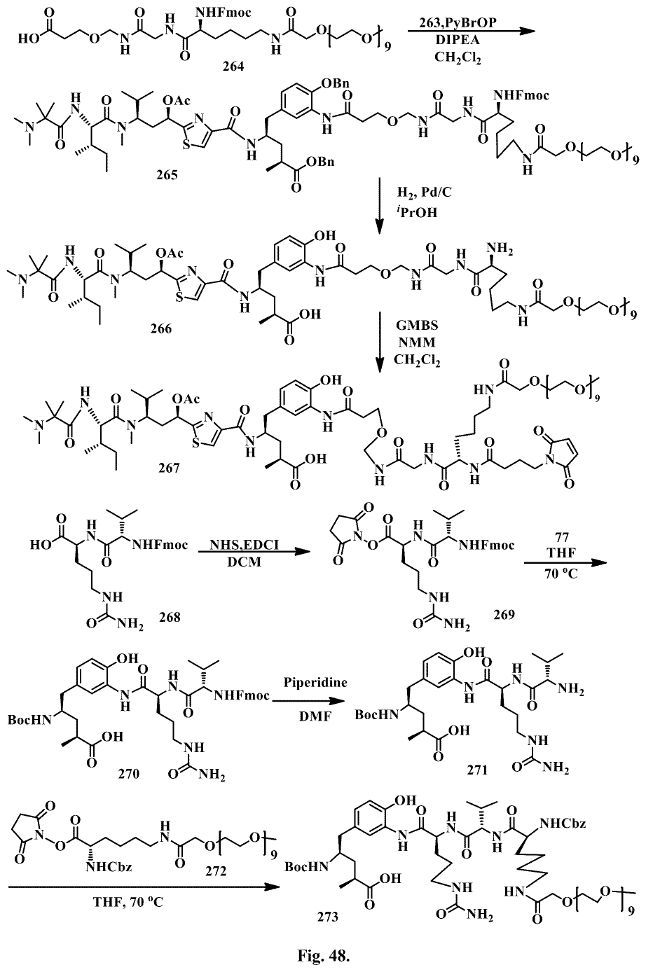

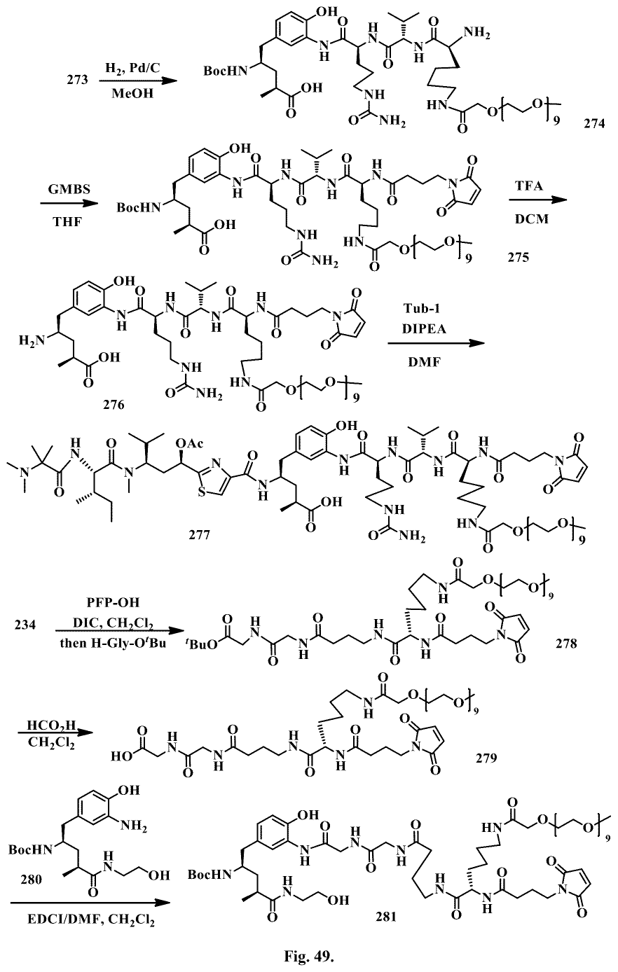

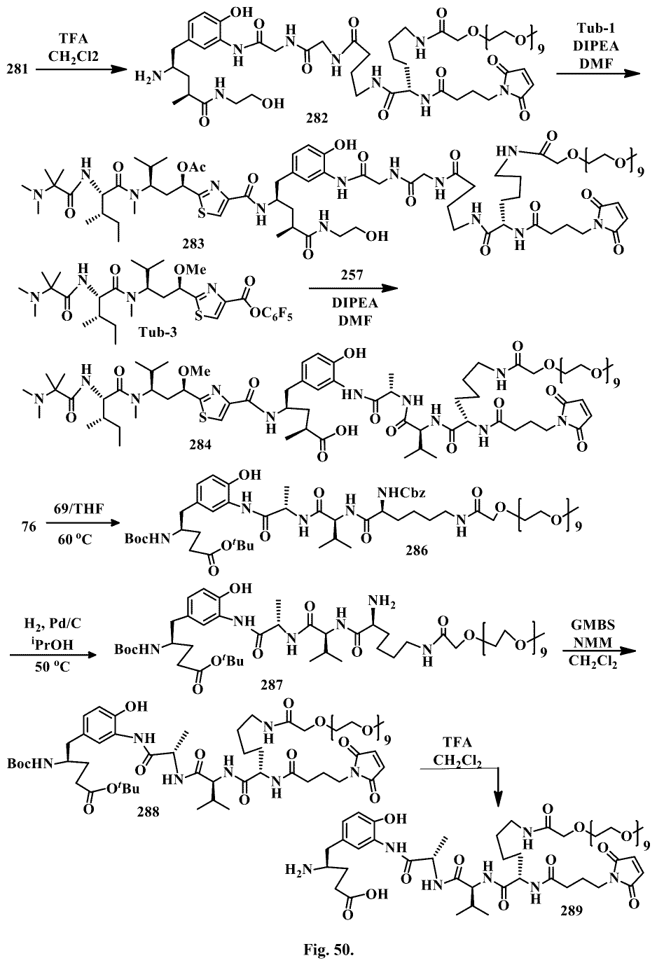

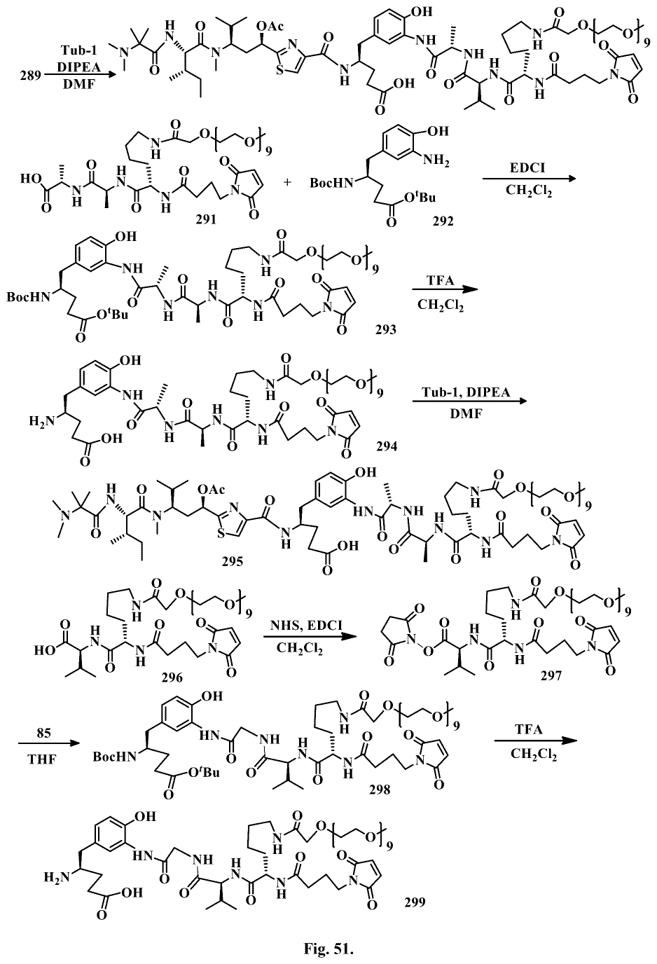

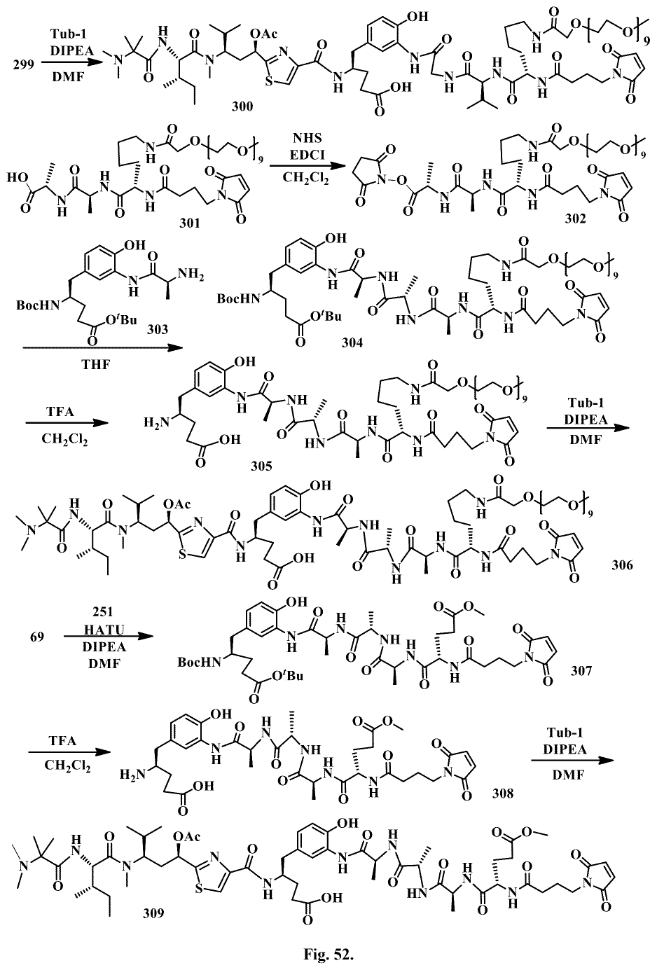

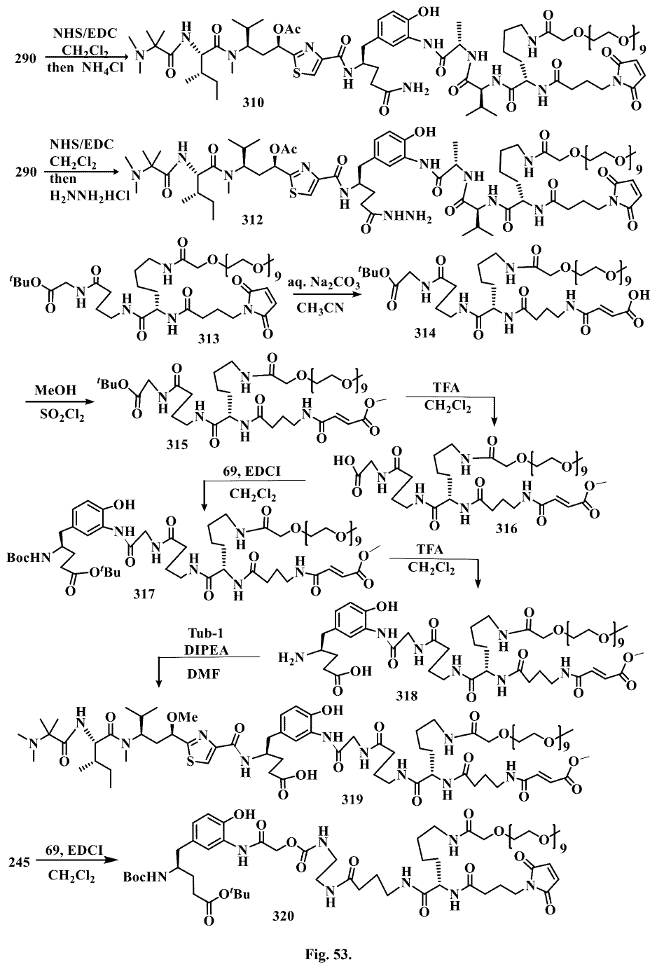

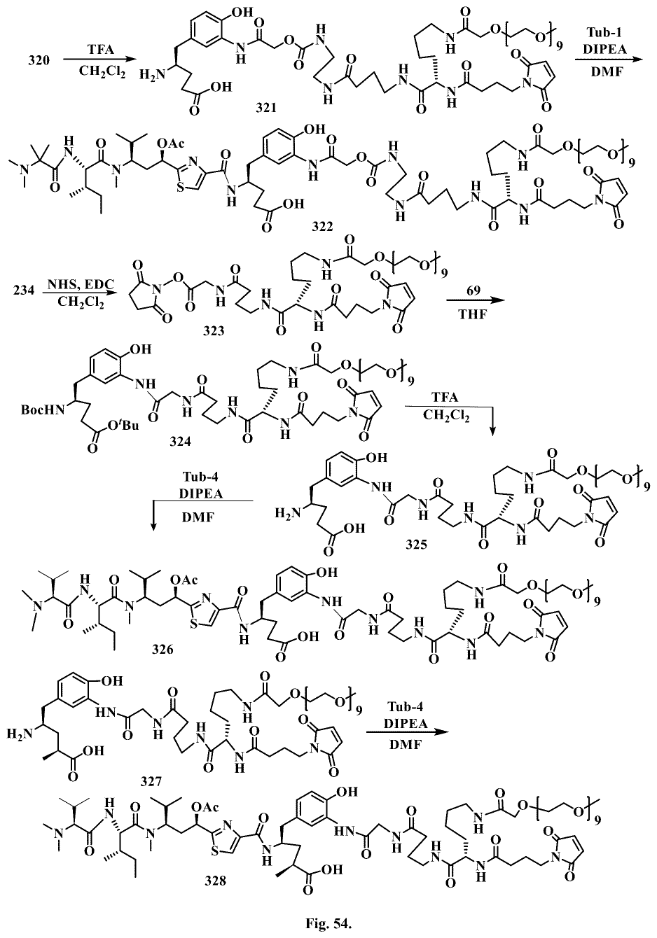

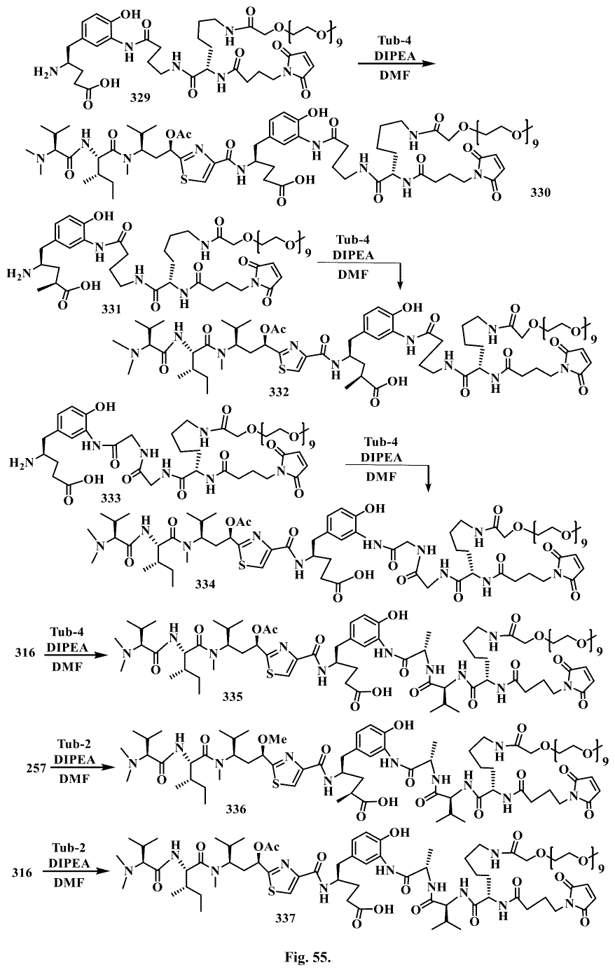

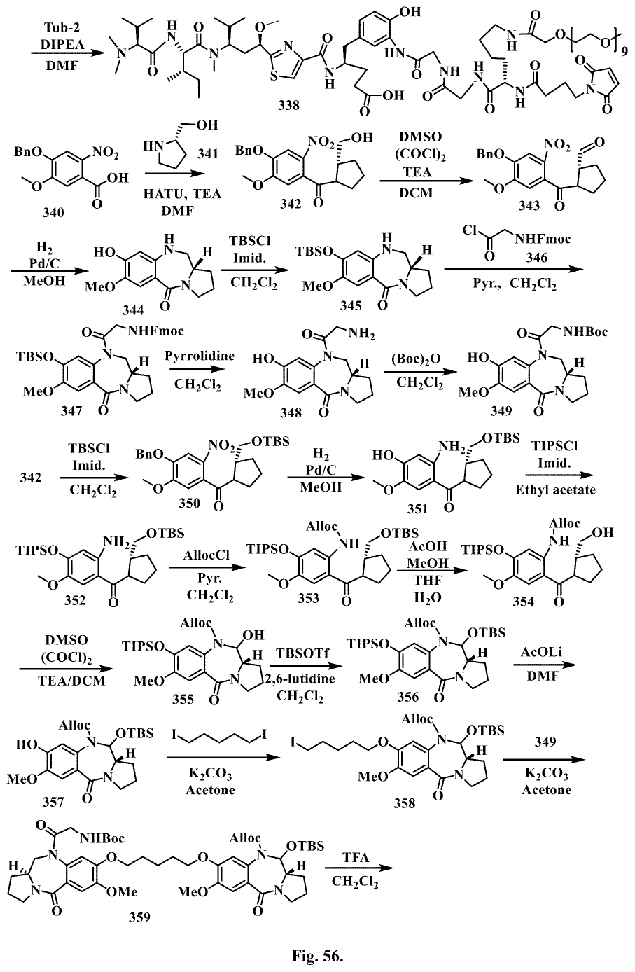

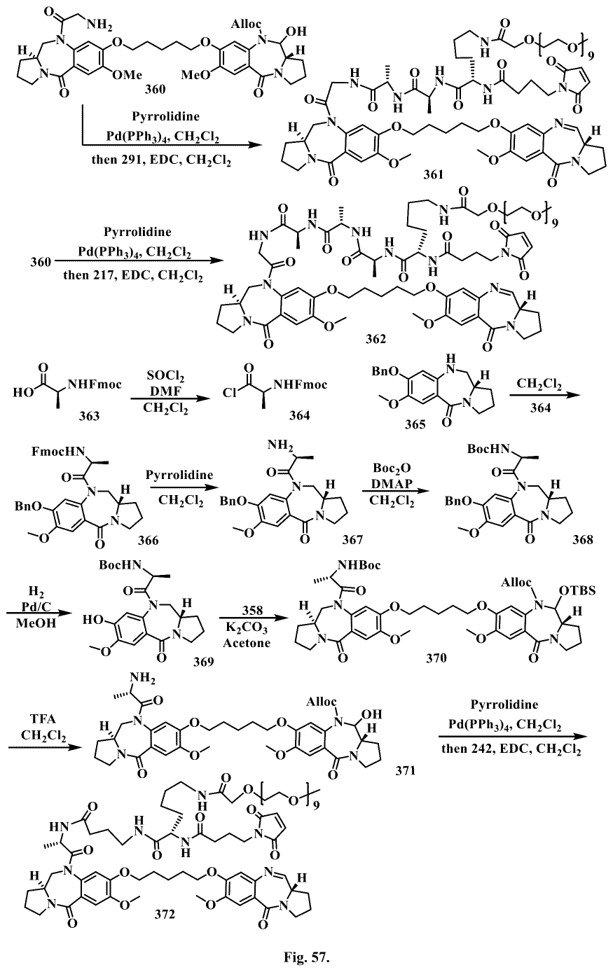

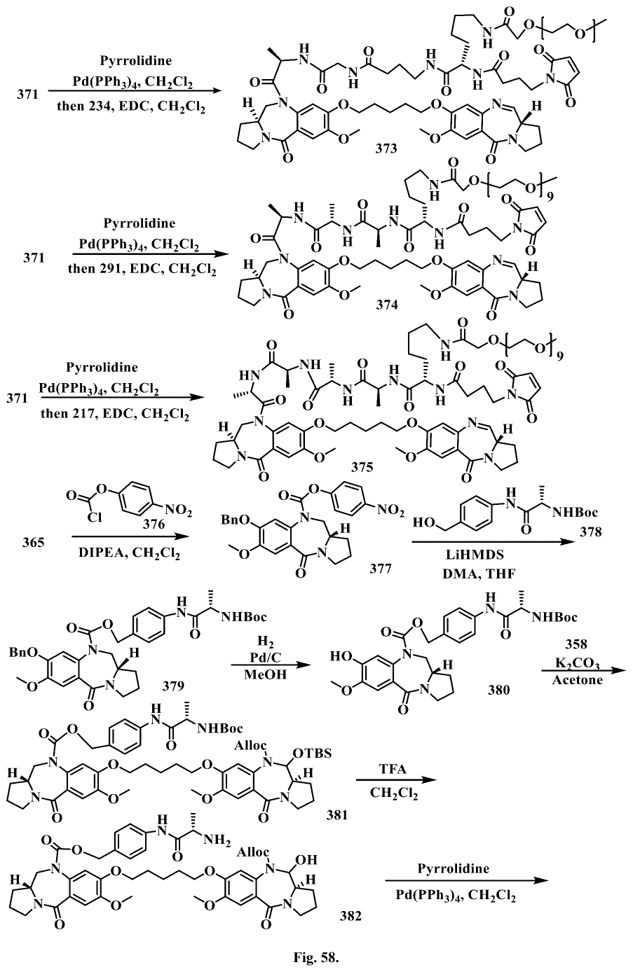

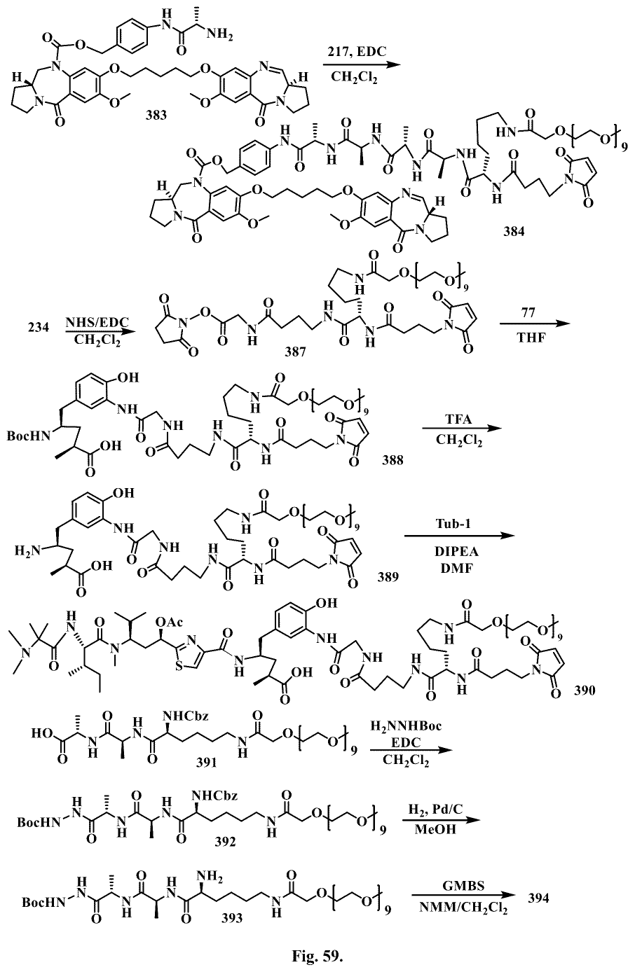

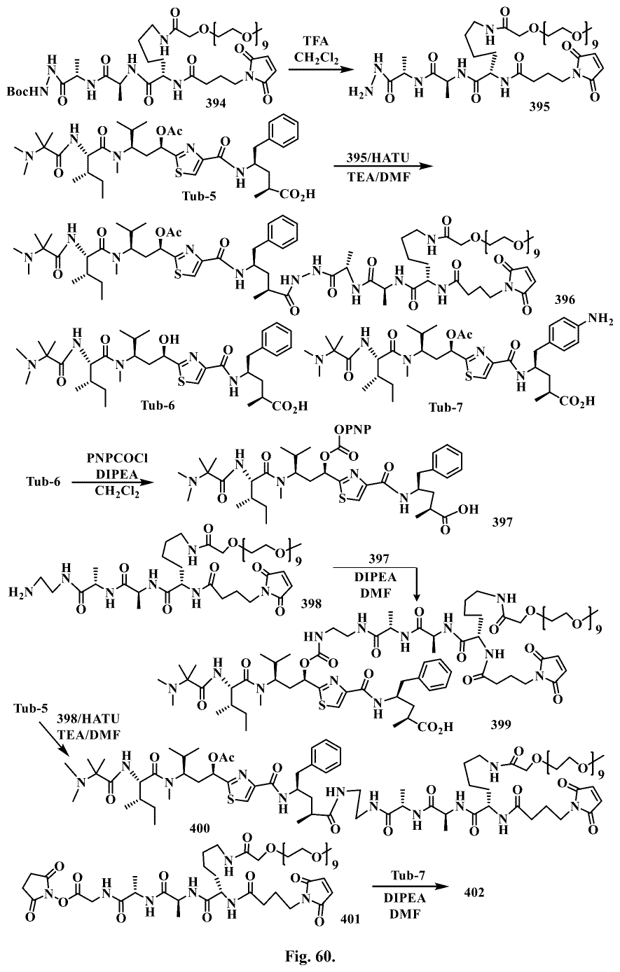

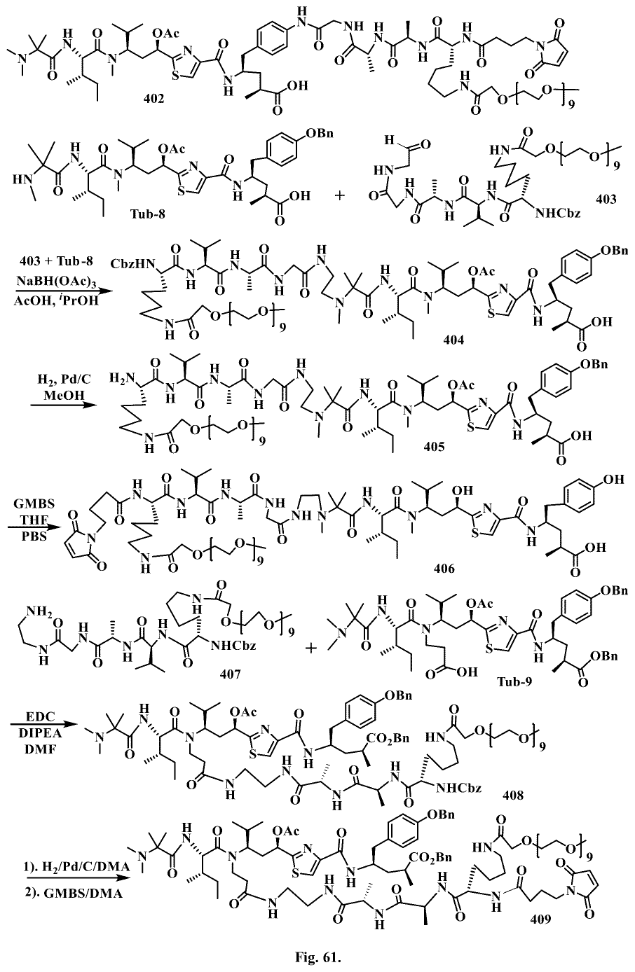

The results were plotted in FIGS. 9-11. All the conjugates did not cause the animal body weight loss at the administered doses. All conjugates demonstrated antitumor activity as comparison with PBS buffer and many of the BCMA conjugates showed better antitumor activities than Belantamabmc-MMAF did.

The sequences mentioned in the description are listed as below:

SEQ-ID-NO: 1-hu5D2-HCDR1
TSFLIHW
SEQ-ID-NO: 2-hu5D2-HCDR2
FIIPGNGGTKYNQKFQ
SEQ-ID-NO: 3-hu5D2-HCDR3
YDGSFEGYFDV
SEQ-ID-NO: 4-hu5D2-LCDR1
SSQSLVHSDGNTYLH
SEQ-ID-NO: 5-hu5D2-LCDR2
KVSNRDS
SEQ-ID-NO: 6-hu5D2-LCDR3
SQSTHWPWT
SEQ-ID-NO: 7-BCMA-extracellular-domain
mlqmagqcsqneyfdsllhacipcqlrcssntppltcqrycnasvtnsvkgtna
SEQ-ID-NO: 8-hu5D2-heavy-chain
qvqlvqsgaevvkpgasvkmsckasgytftsflihwvkqapgqglewigfiipgnggtkynqkfqgkatltsdtssstaymelsslrseds
avyycarydgsfegyfdvwgqgttltvssastkgpsvfplapsskstsggtaalgclvkdyfpepvtvswnsgaltsgvhtfpavlqssglyslssv
vtvpssslgtqtyicnvnhkpsntkvdkkvepkscdkthtcppcpapellggpsvflfppkpkdtlmisrtpevtcvvvdvshedpevkfnwyvd
gvevhnaktkpreeqynstyrvvsvltvlhqdwlngkeykckvsnkalpapiektiskakgqprepqvytlppsreemtknqvsltclvkgfyps
diavewesngqpennykttppvldsdgsfflyskltvdksrwqqgnvfscsvmhealhnhytqkslslspgk
SEQ-ID-NO: 9hu5D2-Light-chain
dvvmtqsplslpvslrqpasiscrssqslvhsdgntylhwylqkpgqsprlliykvsnrdsgvpdrfsgsgsgtdftlkisrveaedlgvyfcs
qsthwpwtfgqgtkleikrtvaapsvfifppsdeqlksgtasvvcllnnfypreakvqwkvdnalqsgnsqesvteqdskdstyslsstltlskady
ekhkvyacevthqglsspvtksfnrgec
SEQ-ID-NO: 10-5D2-VH
evqlqqsgpelvkpgasvkmsckasgytftsflihwvkqkpgqglewigfiipyndgtkynekfkgkatltsdkssstaymelssltsedsavyyc
arydgsfegyfdvwgagttltvssa
SEQ-ID-NO: 11-5D2-VL
dvlmtqtplslpvslrdqasiscrssqslvhsdgntylhwylqkpgqspklliykvsnrfsgvpdrfsgsgsgtdftlkisrveaedlgvyfcsq
sthvpwtfgggtkleik
SEQ-ID-NO: 12-IgG1-constant
stkgpsvfplapsskstsggtaalgclvkdyfpepvtvswnsgaltsgvhtfpavlqssglyslssvvtvpssslgtqtyicnvnhkpsntkvdkkv
epkscdkthtcppcpapellggpsvflfppkpkdtlmisrtpevtcvvvdvshedpevkfnwyvdgvevhnaktkpreeqynstyrvvsvltvlh
qdwlngkeykckvsnkalpapiektiskakgqprepqvytlppsreemtknqvsltclvkgfypsdiavewesngqpennykttppvldsdgsf
flyskltvdksrwqqgnvfscsvmhealhnhytqkslslspgk
SEQ-ID-NO: 13-c5D2-HC
evqlqqsgpelvkpgasvkmsckasgytftsflihwvkqkpgqglewigfiipyndgtkynekfkgkatltsdkssstaymelssltseds
avyycarydgsfegyfdvwgagttltvssastkgpsvfplapsskstsggtaalgclvkdyfpepvtvswnsgaltsgvhtfpavlqssglyslssv
vtvpssslgtqtyicnvnhkpsntkvdkkvepkscdkthtcppcpapellggpsvflfppkpkdtlmisrtpevtcvvvdvshedpevkfnwyvd
gvevhnaktkpreeqynstyrvvsvltvlhqdwlngkeykckvsnkalpapiektiskakgqprepqvytlppsreemtknqvsltclvkgfyps
diavewesngqpennykttppvldsdgsfflyskltvdksrwqqgnvfscsvmhealhnhytqkslslspgk
SEQ-ID-NO: 14-Kappa-constant
tvaapsvfifppsdeqlksgtasvvcllnnfypreakvqwkvdnalqsgnsqesvteqdskdstyslsstltlskadyekhkvyacevthqg
lsspvtksfnrgec
SEQ-ID-NO: 15-c5D2-LC
dvlmtqtplslpvslrdqasiscrssqslvhsdgntylhwylqkpgqspklliykvsnrfsgvpdrfsgsgsgtdftlkisrveaedlgvyfcsq
sthvpwtfgggtkleiktvaapsvfifppsdeqlksgtasvvcllnnfypreakvqwkvdnalqsgnsqesvteqdskdstyslsstltlskadyek
hkvyacevthqglsspvtksfnrgec
SEQ-ID-NO: 16-hu5D2-VH
qvqlvqsgaevvkpgasvkmsckasgytftsflihwvkqapgqglewigfiipgnggtkynqkfqgkatltsdtssstaymelsslrseds
avyycarydgsfegyfdvwgqgttltvssa
SEQ-ID-NO: 17-hu5D2-VL
dvvmtqsplslpvslrqpasiscrssqslvhsdgntylhwylqkpgqsprlliykvsnrdsgvpdrfsgsgsgtdftlkisrveaedlgvyfcs
qsthwpwtfgqgtkleikr
SEQ-ID-NO: 18-hu5D2-heavy-chain
atggagttcggcctgtcctgggtgttcctggtggccctgtttaggggagtgcagtgccaggtgcagctggtgcagagcggagctgaggtggtg
aagccaggagctagcgtgaagatgtcttgtaaggcctccggctacaccttcacatcttttctgatccactgggtgaagcaggctccaggacagggac
tggagtggatcggcttcatcatccctggcaacggcggcaccaagtacaatcagaagtttcagggcaaggccaccctgacatccgacacctccagctc
tacagcttatatggagctgtccagcctgaggtctgaggattccgccgtgtactattgcgctcggtacgacggcagcttcgagggctattttgacgtg
tggggccagggcaccacactgacagtgtctagcgcttctaccaagggaccatccgtgttcccactggccccctccagcaagtccaccagcggaggaa
cagccgctctgggatgcctggtgaaggactacttcccagagcccgtgacagtgagctggaactctggcgctctgacctccggcgtgcacacatttcc
agctgtgctgcagtcttccggcctgtacagcctgagctctgtggtgaccgtgccctccagctctctgggcacccagacatatatctgcaacgtgaat
cacaagccatccaatacaaaggtggacaagaaggtggagcccaagagctgtgataagacccatacatgccccccttgtcctgccccagagctgctgg
gaggaccatccgtgttcctgtttccacccaagcctaaggacaccctgatgatctctaggacccccgaggtgacatgcgtggtggtggacgtgtccca
cgaggaccccgaggtgaagtttaactggtacgtggatggcgtggaggtgcataatgctaagaccaagcctagggaggagcagtacaacagcacctat
cgggtggtgtctgtgctgacagtgctgcaccaggattggctgaacggcaaggagtataagtgcaaggtgagcaataaggccctgcccgctcctatcg
agaagaccatctctaaggccaagggccagcctagagagccacaggtgtacacactgcctccaagccgcgaggagatgaccaagaaccaggtgtctct
gacatgtctggtgaagggcttctatccttctgacatcgctgtggagtgggagtccaatggccagccagagaacaattacaagaccacaccccctgtg
ctggactccgatggcagcttctttctgtattccaagctgaccgtggataagagcagatggcagcagggcaacgtgttttcttgctccgtgatgcatg
aggctctgcacaatcattatacacagaagagcctgtctctgtcccctggcaagtga
SEQ-ID-NO: 19-hu5D2-Light-chain
atggagttcggcctgtcctgggtgttcctggtggccctgtttagaggcgtgcagtgcgacgtggtcatgacacagtcccccctgagcctgcctgt
gagcctgagacagcccgcttctatctcctgtcgctccagccagtctctggtgcattccgatggcaacacctacctgcattggtatctgcagaagcca
ggccagtctccccgcctgctgatctacaaggtgagcaatagggactctggcgtgcctgatcggttcagcggctctggctccggaaccgactttacac
tgaagatctccagggtggaggccgaggatctgggcgtgtatttctgcagccagtctacccactggccatggacatttggccagggcaccaagctgga
gatcaagcggacagtggcggcgccgtccgtgttcatctttccccctagcgacgagcagctgaagagcggcaccgcctctgtggtgtgcctgctgaac
aatttctacccaagggaggccaaggtgcagtggaaggtggataacgctctgcagagcggcaattctcaggagtccgtgaccgagcaggacagcaagg
attctacatattccctgtccagcaccctgacactgtctaaggccgactacgagaagcacaaggtgtatgcttgcgaggtgacccatcagggcctgtc
ttcccccgtgacaaagtcctttaaccggggcgagtgttgatga
SEQ-ID-NO: 20-hu5D2-VH
caggtgcagctggtgcagagcggagctgaggtggtgaagccaggagctagcgtgaagatgtcttgtaaggcctccggctacaccttcacatctt
ttctgatccactgggtgaagcaggctccaggacagggactggagtggatcggcttcatcatccctggcaacggcggcaccaagtacaatcagaagtt
tcagggcaaggccaccctgacatccgacacctccagctctacagcttatatggagctgtccagcctgaggtctgaggattccgccgtgtactattgc
gctcggtacgacggcagcttcgagggctattttgacgtgtggggccagggcaccacactgacagtgtctagcgct
SEQ-ID-NO: 21-hu5D2-VL
gacgtggtcatgacacagtcccccctgagcctgcctgtgagcctgagacagcccgcttctatctcctgtcgctccagccagtctctggtgcattcc
gatggcaacacctacctgcattggtatctgcagaagccaggccagtctccccgcctgctgatctacaaggtgagcaatagggactctggcgtgcctg
atcggttcagcggctctggctccggaaccgactttacactgaagatctccagggtggaggccgaggatctgggcgtgtatttctgcagccagtctac
ccactggccatggacatttggccagggcaccaagctggagatcaag
SEQ-ID-NO: 22-5D2-VH
gaggtccagctccagcagtctggacctgagctggtaaagcctggggcttcagtgaagatgtcctgcaaggcttctggatacacattcactagcttt
cttatacactgggtgaagcagaagcctgggcagggccttgagtggattggatttattattccttacaatgatggtactaagtacaatgagaagttca
aaggcaaggccacactgacttcagacaaatcctccagcacagcctacatggaactcagcagcctgacctctgaagactctgcggtctattactgtgc
aagatatgatggtagcttcgagggttacttcgatgtctggggcgcagggaccactctcacagtctcctcggcc
SEQ-ID-NO: 23-5D2-VL
gacgttttgatgacccagactccactctccctgcctgtcagtcttagagatcaagcctccatctcttgcagatctagtcagagccttgtacacagtg
atggaaacacctatttacattggtacctgcagaagccaggccagtctccaaagctcctgatctacaaagtttccaaccgattttcaggggtcccaga
caggttcagtggcagtggatcagggacagatttcacactcaagatcagcagagtggaggctgaggatctgggagtttatttctgctctcaaagtaca
catgttccgtggacgttcggtggaggcaccaagctggaaatcaaa
SEQ-ID-NO: 24-IgG1-constant
tctaccaagggaccatccgtgttcccactggccccctccagcaagtccaccagcggaggaacagccgctctgggatgcctggtgaaggactac
ttcccagagcccgtgacagtgagctggaactctggcgctctgacctccggcgtgcacacatttccagctgtgctgcagtcttccggcctgtacagcc
tgagctctgtggtgaccgtgccctccagctctctgggcacccagacatatatctgcaacgtgaatcacaagccatccaatacaaaggtggacaagaa
ggtggagcccaagagctgtgataagacccatacatgccccccttgtcctgccccagagctgctgggaggaccatccgtgttcctgtttccacccaag
cctaaggacaccctgatgatctctaggacccccgaggtgacatgcgtggtggtggacgtgtcccacgaggaccccgaggtgaagtttaactggtacg
tggatggcgtggaggtgcataatgctaagaccaagcctagggaggagcagtacaacagcacctatcgggtggtgtctgtgctgacagtgctgcacca
ggattggctgaacggcaaggagtataagtgcaaggtgagcaataaggccctgcccgctcctatcgagaagaccatctctaaggccaagggccagcct
agagagccacaggtgtacacactgcctccaagccgcgaggagatgaccaagaaccaggtgtctctgacatgtctggtgaagggcttctatccttctg
acatcgctgtggagtgggagtccaatggccagccagagaacaattacaagaccacaccccctgtgctggactccgatggcagcttctttctgtattc
caagctgaccgtggataagagcagatggcagcagggcaacgtgttttcttgctccgtgatgcatgaggctctgcacaatcattatacacagaagagc
ctgtctctgtcccctggcaag
SEQ-ID-NO: 25-c5D2-HC
gaggtccagctccagcagtctggacctgagctggtaaagcctggggcttcagtgaagatgtcctgcaaggcttctggatacacattcactagcttt
cttatacactgggtgaagcagaagcctgggcagggccttgagtggattggatttattattccttacaatgatggtactaagtacaatgagaagttca
aaggcaaggccacactgacttcagacaaatcctccagcacagcctacatggaactcagcagcctgacctctgaagactctgcggtctattactgtgc
aagatatgatggtagcttcgagggttacttcgatgtctggggcgcagggaccactctcacagtctcctcggcctctaccaagggaccatccgtgtt
cccactggccccctccagcaagtccaccagcggaggaacagccgctctgggatgcctggtgaaggactacttcccagagcccgtgacagtgagctgg
aactctggcgctctgacctccggcgtgcacacatttccagctgtgctgcagtcttccggcctgtacagcctgagctctgtggtgaccgtgccctcca
gctctctgggcacccagacatatatctgcaacgtgaatcacaagccatccaatacaaaggtggacaagaaggtggagcccaagagctgtgataagac
ccatacatgccccccttgtcctgccccagagctgctgggaggaccatccgtgttcctgtttccacccaagcctaaggacaccctgatgatctctagg
acccccgaggtgacatgcgtggtggtggacgtgtcccacgaggaccccgaggtgaagtttaactggtacgtggatggcgtggaggtgcataatgct
aagaccaagcctagggaggagcagtacaacagcacctatcgggtggtgtctgtgctgacagtgctgcaccaggattggctgaacggcaaggagtat
aagtgcaaggtgagcaataaggccctgcccgctcctatcgagaagaccatctctaaggccaagggccagcctagagagccacaggtgtacacactgc
ctccaagccgcgaggagatgaccaagaaccaggtgtctctgacatgtctggtgaagggcttctatccttctgacatcgctgtggagtgggagtccaa
tggccagccagagaacaattacaagaccacaccccctgtgctggactccgatggcagcttctttctgtattccaagctgaccgtggataagagcaga
tggcagcagggcaacgtgttttcttgctccgtgatgcatgaggctctgcacaatcattatacacagaagagcctgtctctgtcccctggcaag
SEQ-ID-NO: 26-Kappa-constant
cgtgaggtggcggcgccgtccgtgttcatctttccccctagcgacgagcagctgaagagcggcaccgcctctgtggtgtgcctgctgaacaattt
ctacccaagggaggccaaggtgcagtggaaggtggataacgctctgcagagcggcaattctcaggagtccgtgaccgagcaggacagcaaggattc
tacatattccctgtccagcaccctgacactgtctaaggccgactacgagaagcacaaggtgtatgcttgcgaggtgacccatcagggcctgtcttcc
cccgtgacaaagtcctttaaccggggcgagtgt
SEQ-ID-NO: 27-c5D2-LC
gacgttttgatgacccagactccactctccctgcctgtcagtcttagagatcaagcctccatctcttgcagatctagtcagagccttgtacacagtg
atggaaacacctatttacattggtacctgcagaagccaggccagtctccaaagctcctgatctacaaagtttccaaccgattttcaggggtcccaga
caggttcagtggcagtggatcagggacagatttcacactcaagatcagcagagtggaggctgaggatctgggagtttatttctgctctcaaagtaca
catgttccgtggacgttcggtggaggcaccaagctggaaatcaaacgtgaggtggcggcgccgtccgtgttcatctttccccctagcgacgagcagc
tgaagagcggcaccgcctctgtggtgtgcctgctgaacaatttctacccaagggaggccaaggtgcagtggaaggtggataacgctctgcagagcgg
caattctcaggagtccgtgaccgagcaggacagcaaggattctacatattccctgtccagcaccctgacactgtctaaggccgactacgagaagcac
aaggtgtatgcttgcgaggtgacccatcagggcctgtcttcccccgtgacaaagtcctttaaccggggcgagtgt
SEQ-ID-NO: 28-hu5D2-HCDR1
acatcttttctgatccactgg
SEQ-ID-NO: 29-hu5D2-HCDR2
ttcatcatccctggcaacggcggcaccaagtacaatcagaagtttcag
SEQ-ID-NO: 30-hu5D2-HCDR3
tacgacggcagcttcgagggctattttgacgtg
SEQ-ID-NO: 31-hu5D2-LCDR1
tccagccagtctctggtgcattccgatggcaacacctacctgcat
SEQ-ID-NO: 32-hu5D2-LCDR2
aaggtgagcaatagggactct
SEQ-ID-NO: 33-hu5D2-LCDR3
Agccagtctacccactggccatggaca
SEQ ID NO: 34
EEQYNSTYR
SEQ ID NO: 35
THTCPPCPAPELLGGPSVFLFPPKPK

1-30. (canceled)

31. An isolated monoclonal antibody or antigen binding fragment thereof which binds B cell maturation antigen (BCMA or CD 269), wherein said antibody or antigen binding fragment thereof comprises: (i) a VH domain, wherein said VH domain comprises CDR H1 comprising SEQ ID No. 1 (TSFLIHW), CDR H2 comprising SEQ ID No. 2 (FIIPGNGGTKYNQKFQ) and CDR H3 comprising SEQ ID No. 3 (YDGSFEGYFDV), and (ii) a VL domain, wherein said VL domain comprises CDR L1 comprises SEQ ID No. 4 (SSQSLVHSDGNTYLH), CDR L2 comprises SEQ ID No. 5 (KVSNRDS) and CDR L3 comprises SEQ ID No. 6 (SQSTHWPWT).

32. The method of claim 59, wherein the cancer of plasma cells is multiple myeloma, plasmacytoma, Waldenstrom macroglobulinemia or plasma cell leukemia, or wherein the cancer of B lymphocytes is Hodgkin's disease.

33. The isolated monoclonal antibody or antigen binding fragment thereof of claim 31, wherein the VL and VH domains are fused to human Ig kappa (SEQ ID NO: 14 or encoded by SEQ ID NO: 26) and IgG1 (SEQ ID NO:12 or encoded by SEQ ID NO:24) constant domains, respectively.

34. The isolated monoclonal antibody or antigen binding fragment thereof of claim 31, wherein the isolated monoclonal antibody or antigen binding fragment thereof comprises a VH domain comprising SEQ ID NO:10, or SEQ ID NO: 16, and a VL domain comprising SEQ ID NO:11 or SEQ ID NO:17; or
the isolated monoclonal antibody or antigen binding fragment thereof comprises a heavy chain comprising SEQ ID NO:8, or SEQ ID NO:13;
the isolated monoclonal antibody or antigen binding fragment thereof comprises a light chain comprising SEQ ID NO:9, or SEQ ID NO:15.

35. The isolated monoclonal antibody or antigen binding fragment thereof of claim 31, wherein the isolated monoclonal antibody or antigen binding fragment thereof is a chimeric, humanized, partially humanized, bi-specific or a single chain antibody, or combination thereof.

36. The isolated monoclonal antibody or antigen binding fragment thereof of claim 31, wherein the isolated monoclonal antibody or antigen binding fragment thereof comprises: (i) a VH domain comprising a sequence encoded by SEQ. ID. NO:20 or SEQ. ID. NO:22, and (ii) a VL domain comprising a sequence encoded by SEQ. ID. NO:21 or SEQ. ID. NO:23; or
the isolated monoclonal antibody or antigen binding fragment thereof comprises a heavy chain encoded by SEQ ID NO:18, or SEQ ID NO:25;
the isolated monoclonal antibody or antigen binding fragment thereof comprises a light chain encoded by SEQ ID NO:19, or SEQ ID NO:27.

37. The isolated monoclonal antibody or antigen binding fragment thereof of claim 31, wherein the isolated monoclonal antibody or antigen binding fragment thereof is glycosylated.

38. The isolated monoclonal antibody or antigen binding fragment thereof of claim 37, wherein the isolated monoclonal antibody or antigen binding fragment thereof comprises an N-linked oligosaccharide chain at Asn300 of the heavy chain.

39. An antibody-drug conjugate (ADC) comprising a monoclonal antibody, or an antigen-binding fragment thereof, directed against B-cell maturation antigen (BCMA) conjugated to a cytotoxin, wherein the monoclonal antibody comprises (a) a heavy chain variable region comprising a complementarity determining region 1 (CDRH1) amino acid sequence of SEQ ID NO: 1, a CDR H2 amino acid sequence of SEQ ID NO: 2, and a CDR H3 amino acid sequence of SEQ ID NO: 3 and (b) a light chain variable region comprising a complementarity determining region 1 (CDR L1) amino acid sequence of SEQ ID NO: 4, a CDR L2 amino acid sequence of SEQ ID NO: 5, and a CDR L3 amino acid sequence of SEQ ID NO: 6.

40. The antibody-drug conjugate of claim 39, has a formula of 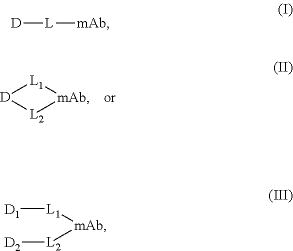
wherein D, D₁ and D₂ are a small molecule cytotoxin or a functional small molecule, in general called payload; L, L₁ and L₂ are a linker; and mAb is the monoclonal antibody or antigen binding fragment thereof.

41. The antibody-drug conjugate of claim 40, wherein the cytotoxin or a functional small molecule D, D₁ and D₂ are independently selected from the group consisting of:
(1) A chemotherapeutic agent selected from the group consisting of:
a). an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD)dimers, tomaymycindimers, indolinobenzodiazepinedimers, imidazobenzothiadiazepinedimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). A plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, DM21, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; erbulins, a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid}; and Inhibitors of nicotinamide phosphoribosyltransferase (NAMPT);
e). A hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (comprising CB 1093, EB 1089 KH 1060, cholecalciferol, ergocalciferol); Photodynamic therapies: (comprising verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. Vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group consisting of olaparib, niraparib, iniparib, talazoparib, veliparib, CEP 9722 (Cephalon's), E7016 (Eisai's), BGB-290 (BeiGene's), or 3-aminobenzamide;
h). An antibiotic, selected from the group consisting of an enediyne antibiotic (selected from the group consisting of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins and carfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (7eclometha), idarubicin, epirubicin, pirarubicin, zorubicin, mtoxantrone, MDR inhibitors or verapamil, Ca²⁺ ATPase inhibitors or thapsigargin, Histone deacetylase inhibitors ((comprising Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; 8eclometha; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; razoxane; rhizoxin; sizofiran; spirogermanium; tenuazonic acid; triaziquone; 2, 2′,2″-trichlorotriethylamine; trichothecenes (including the group consisting of T-2 toxin, verrucarin A, roridin A and anguidine); urethane, siRNA, antisense drugs;
(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, 8eclomethasone dipropionate),DHEA, enanercept, hydroxychloroquine, infliximab, meloxicam, methotrexate, mofetil, mycophenylate, prednisone, sirolimus, tacrolimus;
(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem/cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). Glycopeptides: bleomycin, vancomycin (oritavancin, telavancin), teicoplanin (dalbavancin), ramoplanin;
g). Glycylcyclines: tigecycline;
h). β-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU),3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC),1-nucleosides (including the group consisting of β-1-thymidine and β-1-2′-deoxycytidine), penciclovir, racivir, ribavirin, stampidine, stavudine (d4T), taribavirin (viramidine),telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5). A radioisotope that can be selected from the group consisting of (radionuclides) ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi;
(6). A chromophore molecule, which is capable of absorbing UV light, florescent light, IR light, near IR light, visual light; A class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; Non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170). Acridine derivatives (comprising proflavin, acridine orange, acridine yellow). Arylmethine derivatives (comprising auramine, crystal violet, malachite green). Tetrapyrrole derivatives (comprising porphin, phthalocyanine, bilirubin); Any analogs and derivatives of the following fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; A fluorophore compound: comprising DCFH (2′7′Dichorodihydro-fluorescein, oxidized form), DHR (Dihydrorhodamine 123, oxidized form, light catalyzes oxidation), Fluo-3 (AM ester. pH>6), Fluo-4 (AM ester. pH 7.2), Indo-1 (AM ester, low/high calcium (Ca2+)), SNARF(pH 6/9), Allophycocyanin(APC), AmCyan1 (tetramer, Clontech), AsRed2 (tetramer, Clontech), Azami Green (monomer), Azurite, B-phycoerythrin(BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin(RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreen1 (tetramer), ZsYellow1 (tetramer) and their derivatives;
(7). cell-binding ligands or receptor agonists, which can be selected from Folate derivatives; Glutamic acid urea derivatives; Somatostatin and its analogs(selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH)agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gy-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gy-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of αvβ₁, αvβ₃, αvβ₅, αvβ₆, α₆β₄, α₇β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, abispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists;
(8). pharmaceutically acceptable salts, acids, derivatives, hydrate or hydrated salt; or a crystalline structure; or an optical isomer, racemate, diastereomer or enantiomer of any of the above drugs.

42. The antibody-drug conjugate of claim 40, wherein the cytotoxin D, D₁ and D₂ are independently an anti-microtubule agent, DNA minor groove binder, an RNA polymerase II inhibitor, DNA topoisomerase I or II inhibitor, or a DNA alkylating agent, selected from tubulysins, amanitins, auristatins (including its analogs of AFP, MMAF, MMAE, AEB, AEVB, E), calicheamicin, camptothecins (including SN-38, topotecans or exatecans), etoposides, teniposides, daunomycins, doxorubicins (including morpholino-doxorubicins, cyanomorpholino-doxorubicins), duocarmycins (including CC1065 analogs, DC1, DC4, CBI-dimers), dolastatins, enediynes, eribulin, lexitropsins, taxanes, puromycins, maytansinoids, vinca alkaloids, paclitaxels, docetaxels, pyrrolobenzodiazepines (including PBDs, IGN), rhizoxins, dolastatins, echinomycins, combretatstatin, vinca alkaloid, chalicheamicins, maytansine (DM1, DM4, DM21), geldanamycin, vinblastine, methotrexate, hemiasterlin, netropsin, inhibitor of nicotinamide phosphoribosyltransferases (NAMPT), spliceostatin, pladienolide, protein kinase inhibitor, MEK inhibitor, proteinase inhibitor, immunotoxin, cell receptor agonist or their derivatives or analogs above thereof;
wherein:
(a) Tubulysin and its analogs having the following formula (IVa): 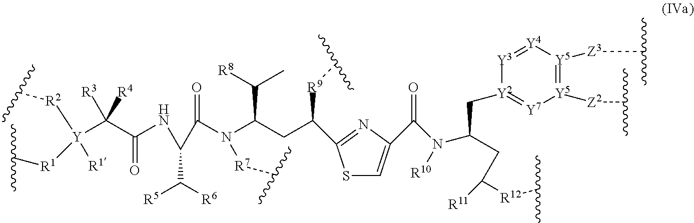
or a pharmaceutically acceptable salt, hydrates, or hydrated salt; or a polymorphic crystalline structure; or an optical isomer, racemate, diastereomer or enantiomer thereof,
wherein [symbol] is a linkage site that either one or two of them can link to L₁ and/or L₂ independently; when two of “ [symbol] ” link to both L₁ and L₂, R¹ and R², or Z² and Z³ are dual linkage sites;
wherein R¹, R^{1′}, R², R³, and R⁴are independently H, C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or R¹R², R¹R³, R²R³, R³R⁴, or R⁵R⁶ form a 3-7 membered carbocyclic, cycloalkyl, heterocyclic, heterocycloalkyl, aromatic or heteroaromatic ring system; R¹ and R² can be independently absent when they link to L₁ or L₂ independently or simultaneously, Y¹ is N or CH;
wherein R⁵, R⁶, R⁸, R¹⁰ and R¹¹ are independently H, or C₁˜C₄ alkyl or heteroalkyl;
wherein R⁷ is independently H, R¹⁴, —R¹⁴C(═O)X¹R¹⁵; or —R¹⁴X¹R¹⁵; X¹ is O, S, S—S, NH, CH₂ or NR¹⁴;
wherein R⁹ is selected from H, OH, ═O, —OR¹⁴, —OC(═O)R¹⁴, —OC(═O)NHR¹⁴, —OC(═O)NR¹⁴R¹⁵, OP(═O)(OR¹⁴)₂, —OC(═O)NR¹⁴R¹⁵, or OR¹⁴OP(═O)(OR¹⁵)₂; when R⁹ links L₁ or L₂, R⁹ is, —O—, —OC(═O)NH— or —OC(═O)N(R¹⁴)—;
wherein R¹¹ is independently H, R¹⁴, —R¹⁴C(═O)R¹⁵, —R¹⁴C(═O)X²R¹⁵, wherein X² is —O—, —S—, —NH—, or —N(R¹⁴)—;
wherein R¹² is —COOH, —COSH, —CONH₂, CONHNH₂, CONHNHR¹⁵, —CONH(R¹⁵), —COOR¹⁵, —R¹⁵COR¹⁶, —R¹⁵COOR¹⁶, —R¹⁵C(O)NH₂, —R¹⁵C(O)NHR¹⁶, —COSR¹⁵, R¹⁵S(═O)₂R¹⁶, —R¹⁵P(═O)(OR¹⁷)₂, —R¹⁵OP(═O)(OR¹⁷)₂, —COOCH₂OP(═O)(OR¹⁷)₂, —COX²SO₂R¹⁷, —COOR¹⁵X²R¹⁶, tetrazole, imidazole, or triazole, where X² is —O—, —S—, —NH—, —N(R¹⁵)—, —O—R¹⁵—, —S—R¹⁵—, CH₂ or —NHR¹⁵—; when R¹² links L₁ or L₂, R¹² is —C(O)O—, —C(O)NH—, —C(═O)NHS(O)₂R¹⁵— or —C(═O)N(R¹⁵)—;
R¹³ and R¹⁴ are independently C₁˜C₈ alkyl, heteroalkyl; C₂-C₈ of alkenyl, alkynyl, heteroalkyl, heterocycloalkyl; C₃-C₈ of aryl, Ar-alkyl;
Z² and Z³ are independently H, O, S, NH, N(R¹⁵), NHNH, —OH, —SH, —NH₂, NH, NHNH₂, —NH(R¹⁵), —OR¹⁵, CO, —COX², —COX²R¹⁶, R¹⁷, F, Cl, Br, I, SR¹⁶, NR¹⁶R¹⁷, N═NR¹⁶, N═R¹⁶, NO₂, SOR¹⁶R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷; —O—(C₄-C₁₂ glycoside), —N—(C₄-C₁₂ glycoside); C₁˜C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)_{1˜8}, or CO(Aa)_{1˜8} (which are respectively N-terminal or C-terminal 1-8 the same or different amino acids)), or polyethyleneoxy unit of formula (OCH₂CH₂)ₚ or (OCH₂CH(CH₃))ₚ, wherein p is an integer from 0 to about 1000, or combination of above groups thereof, X² is O, S, S—S, NH, CH₂, OH, SH, NH₂, CHR¹⁵ or NR¹⁵;
R¹⁵, R¹⁶ and R¹⁷ are independently H, C₁˜C₈ alkyl, heteroalkyl; C₂-C₈ of alkenyl, alkynyl, heteroalkyl, heterocycloalkyl; C₃-C₈ of aryl, Ar-alkyl, cycloalkyl, heteroalkylcycloalkyl, alkylcarbonyl, heteroaryl, alkylcarbonyl, or Na⁺, K⁺, Cs⁺, Li⁺, Ca²⁺, Mg⁺, Zn²⁺, N⁺(R¹)(R²)(R³)(R⁴), HN⁺(C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N (R¹), thus Y², Y⁴, Y⁵, Y⁶ and Y⁷form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y⁴, Y⁵, Y⁶ and Y⁷ form aromatic ring of benzene, pyridine, pyridazine, pyrimidine, pyrazine, triazine, tetrazine, pentazine;
(a) the tubulysin analogs of Formula (IVa) specifically having the structures shown below: 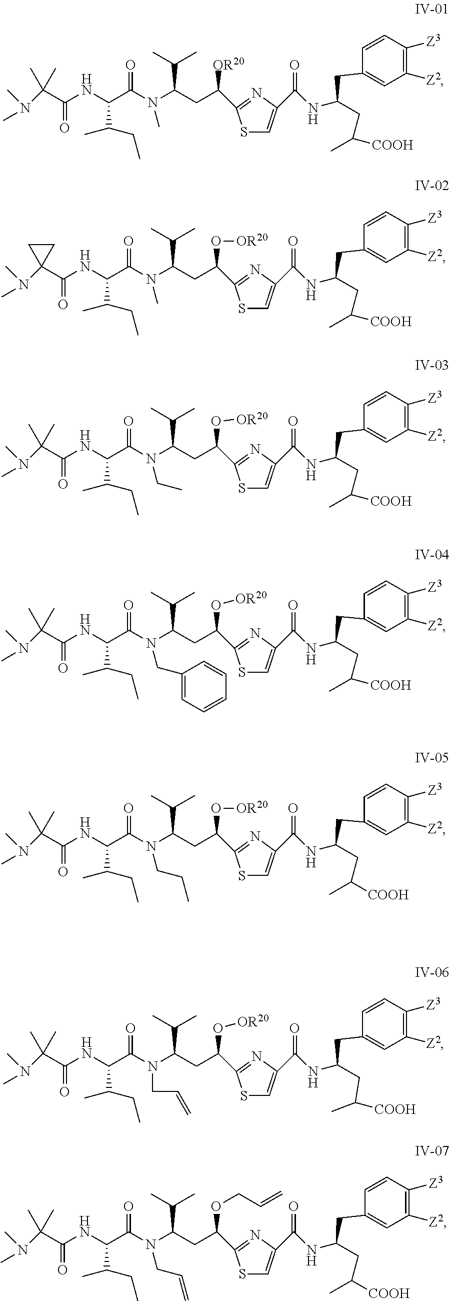 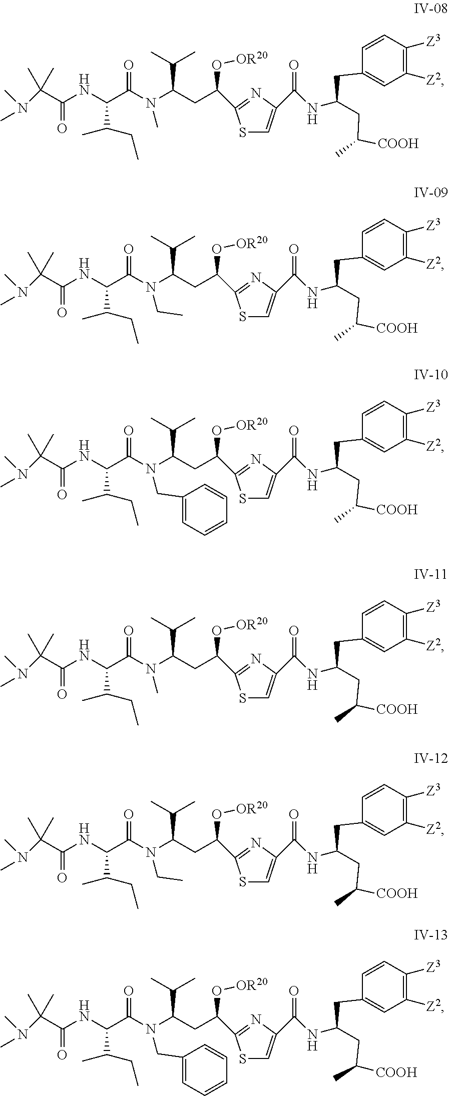 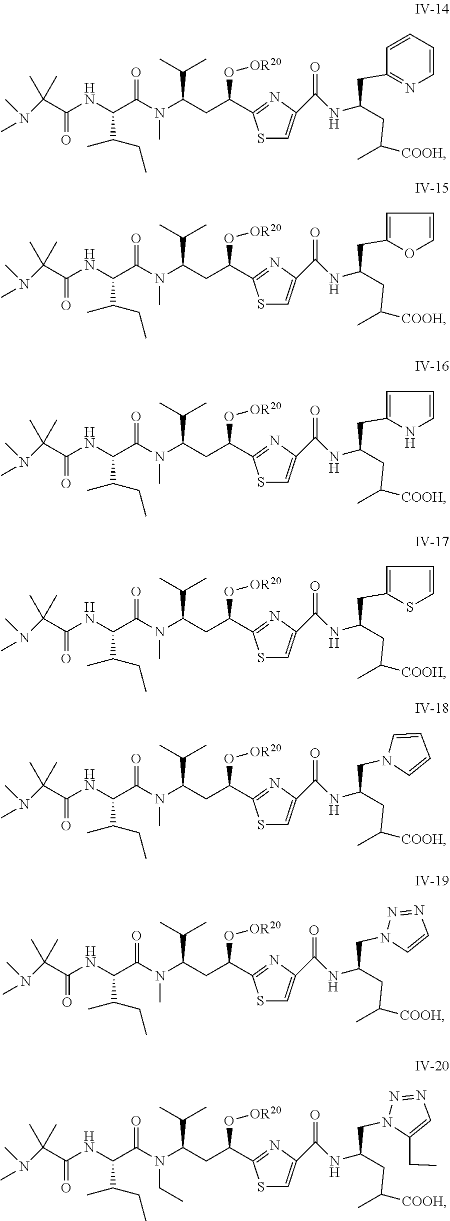 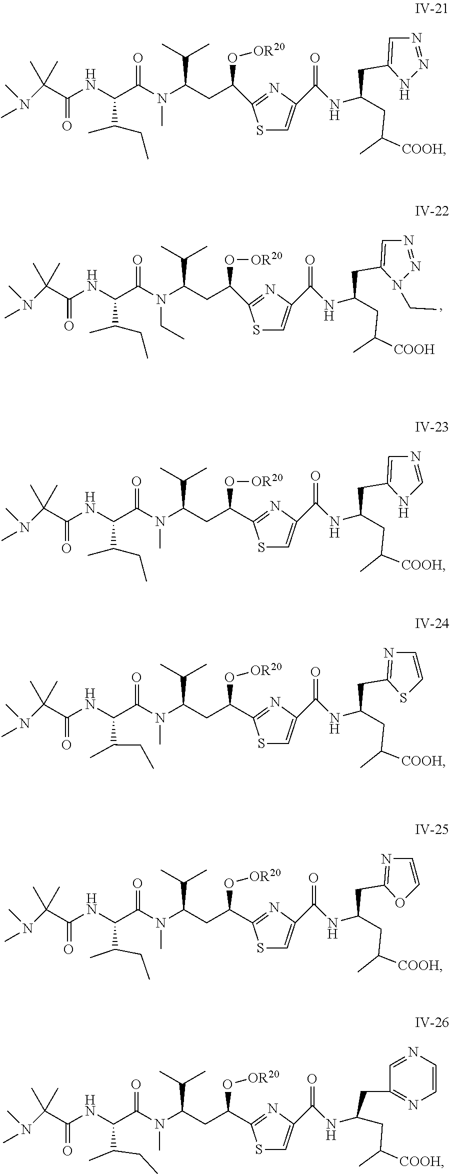 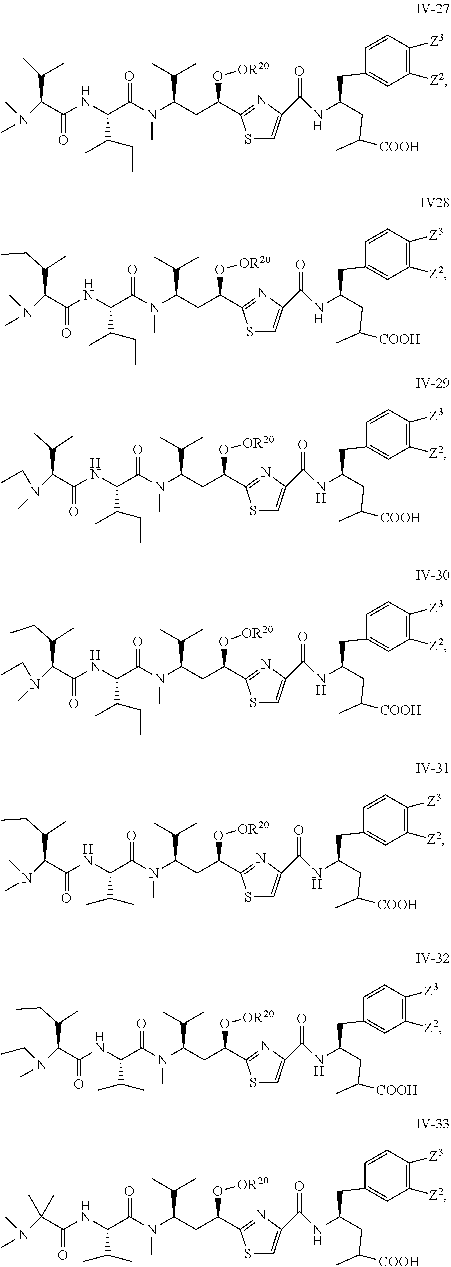 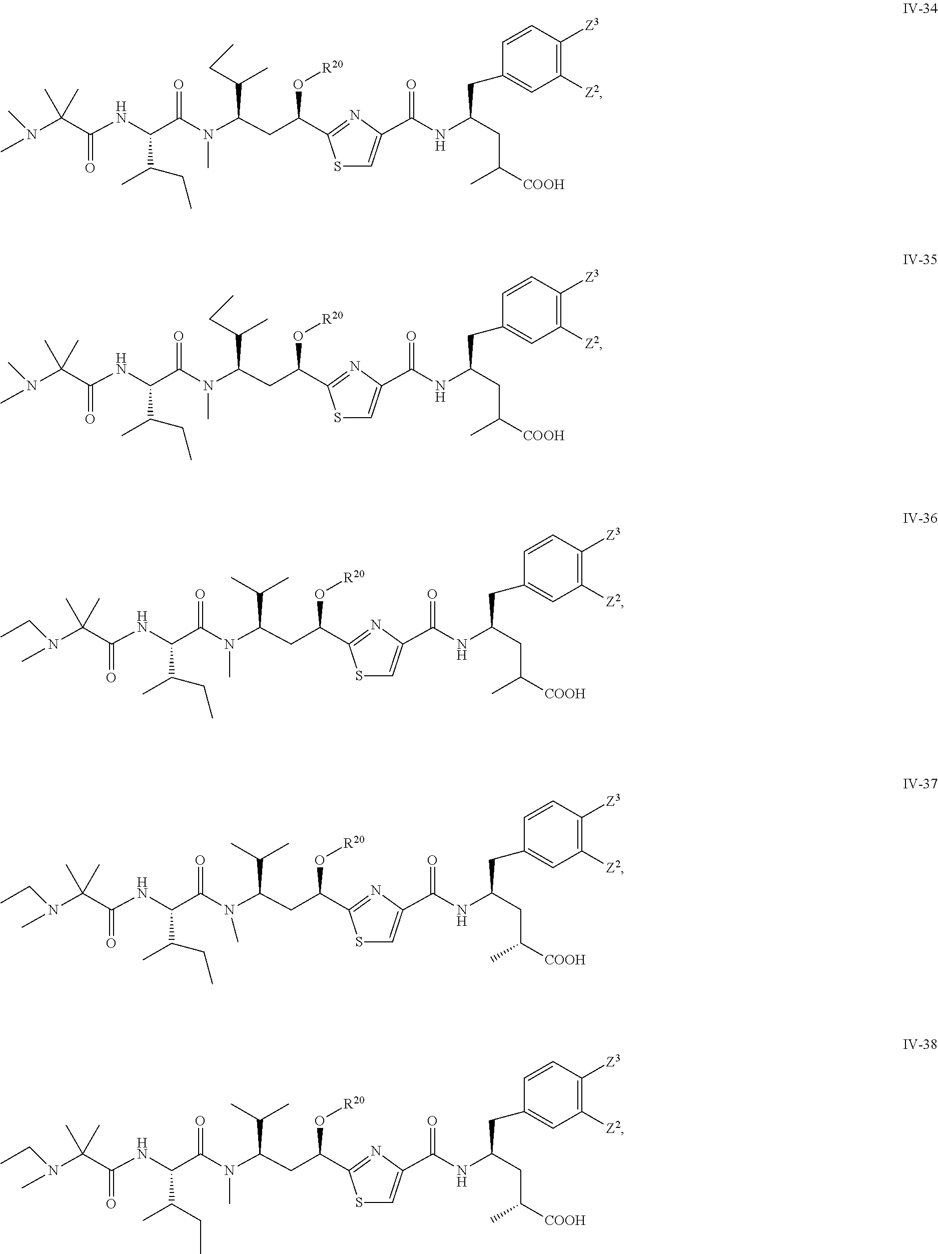 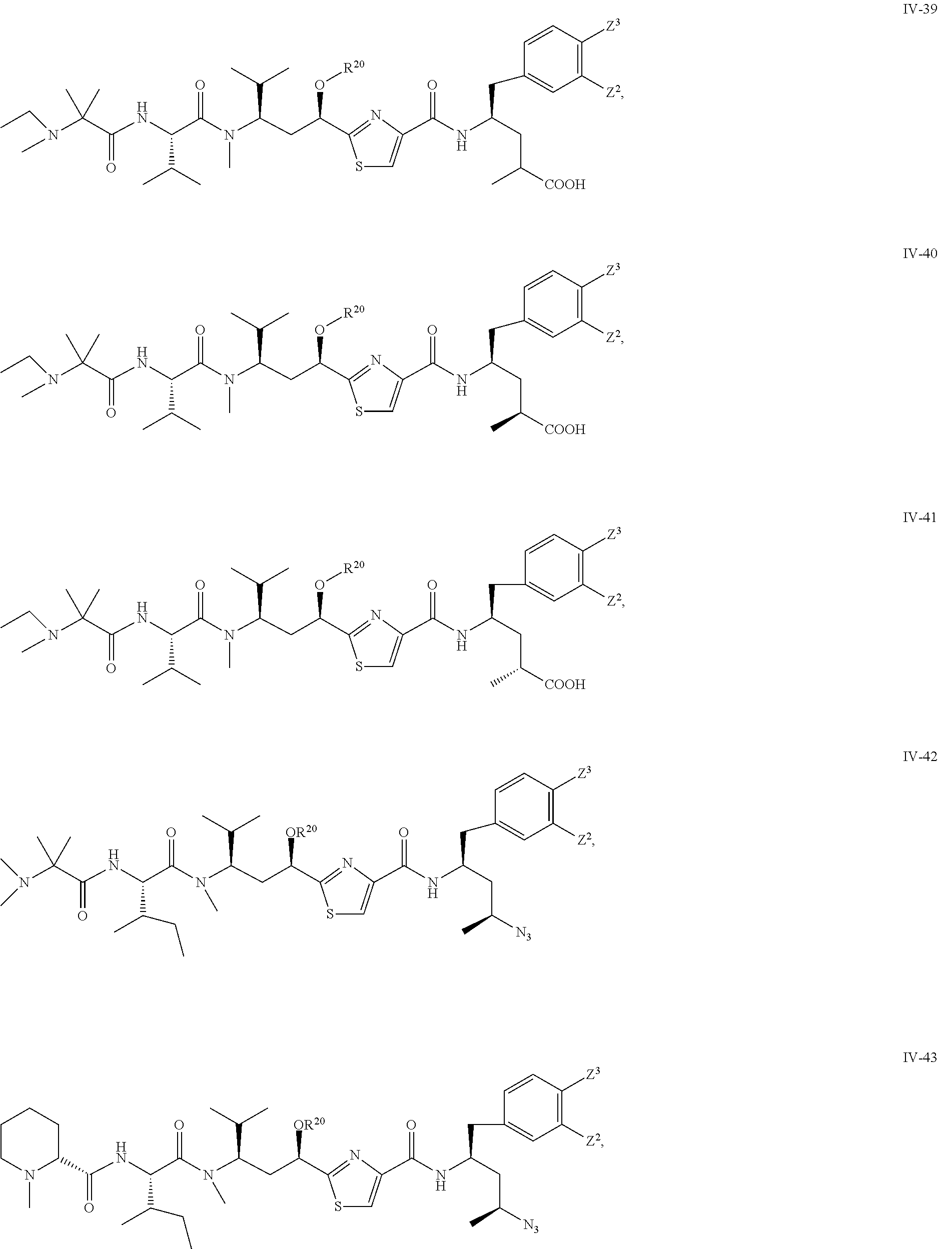 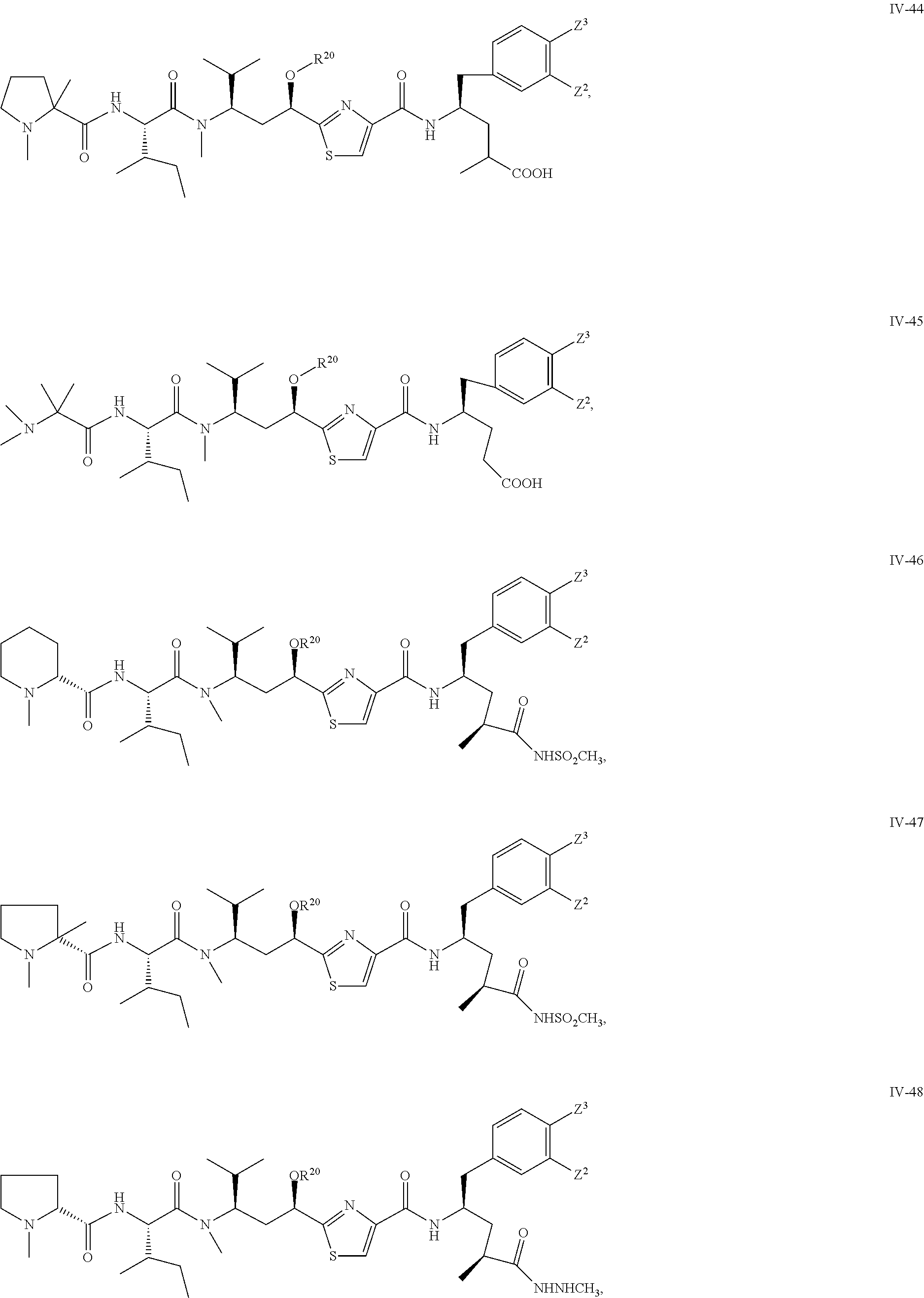 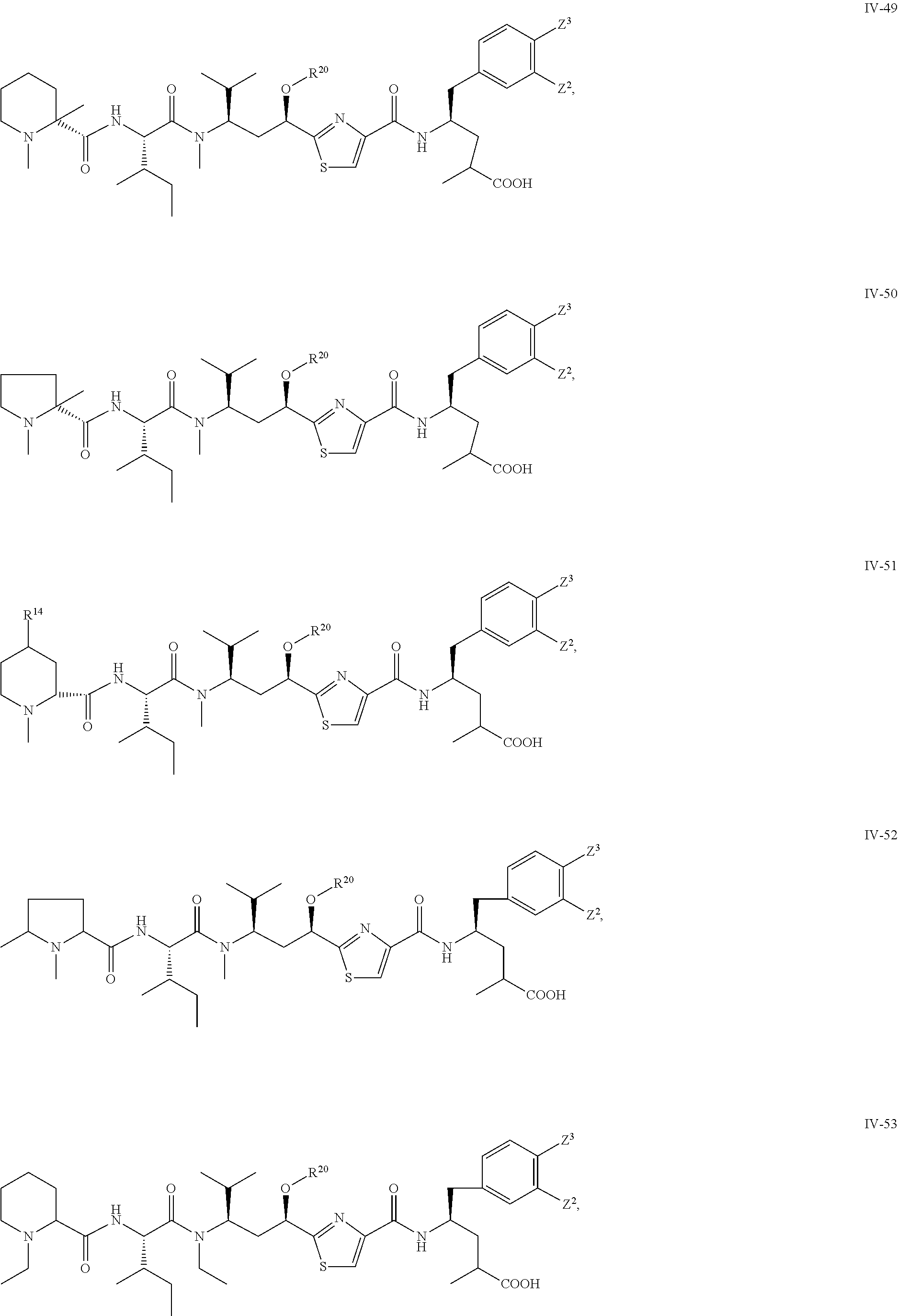 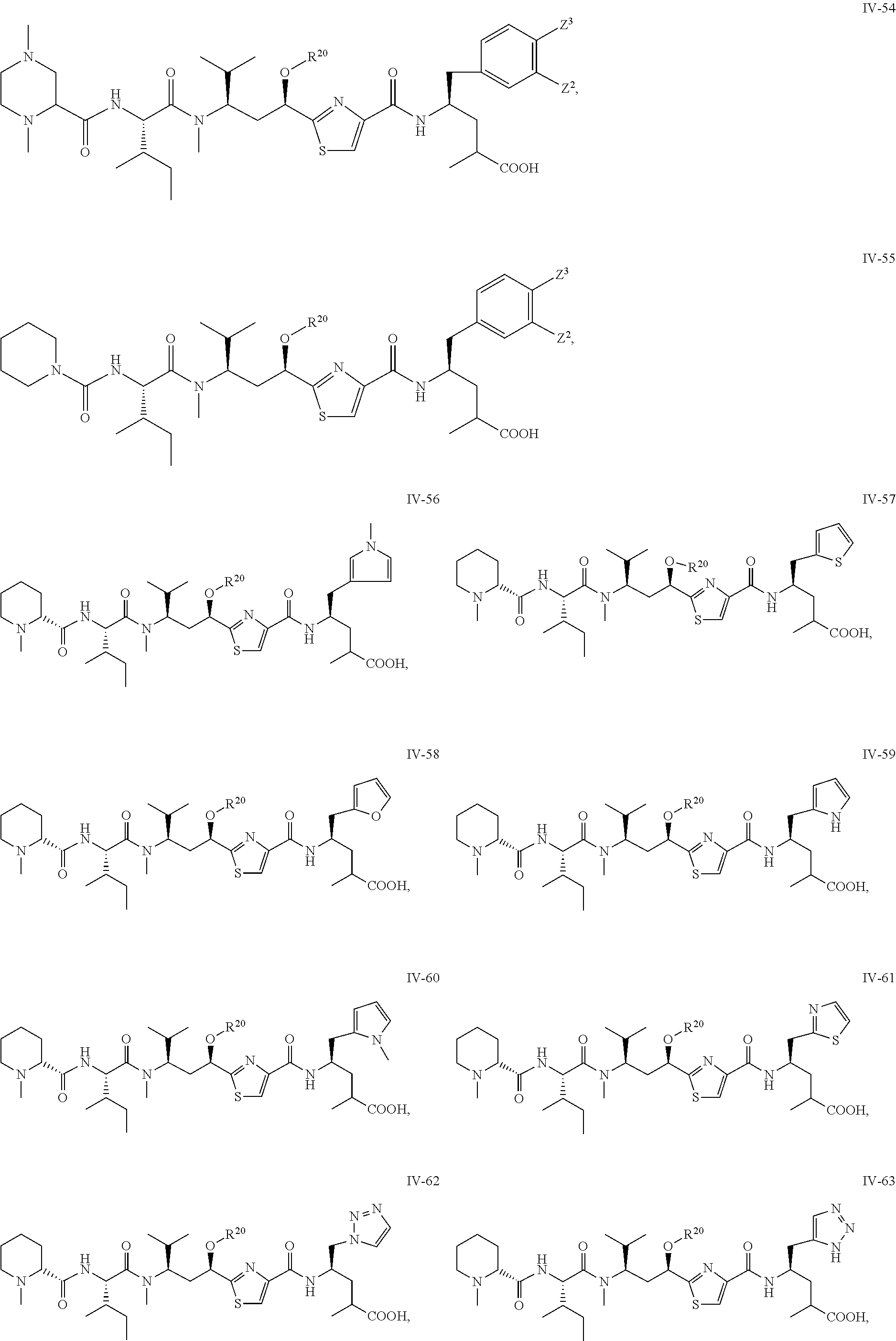 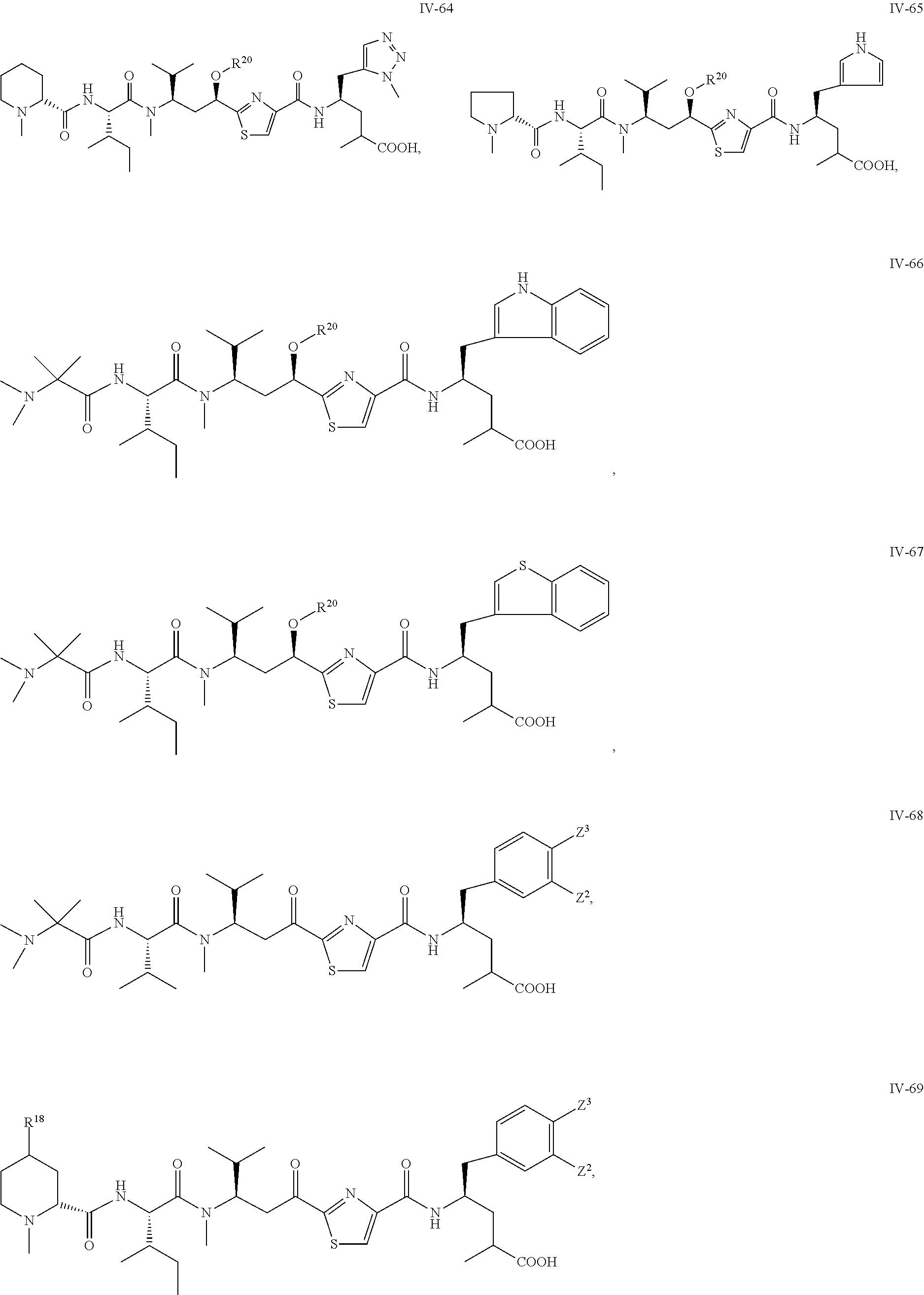 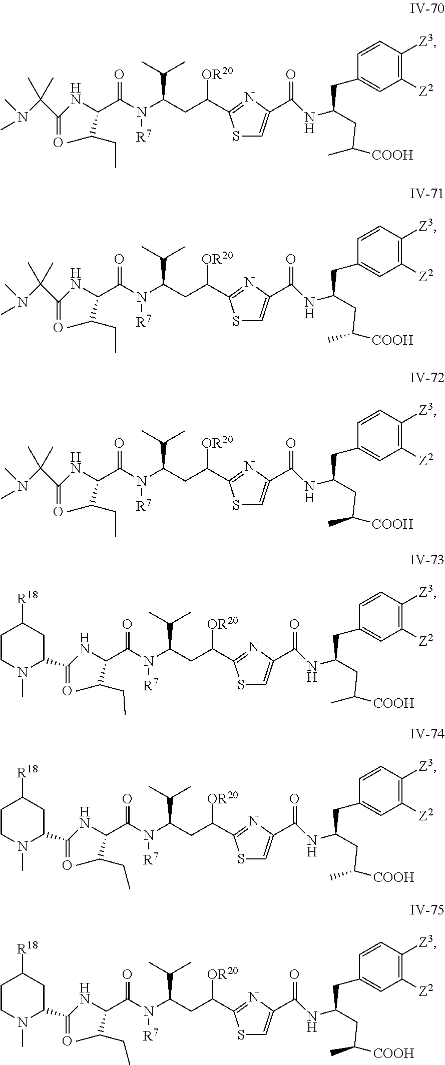 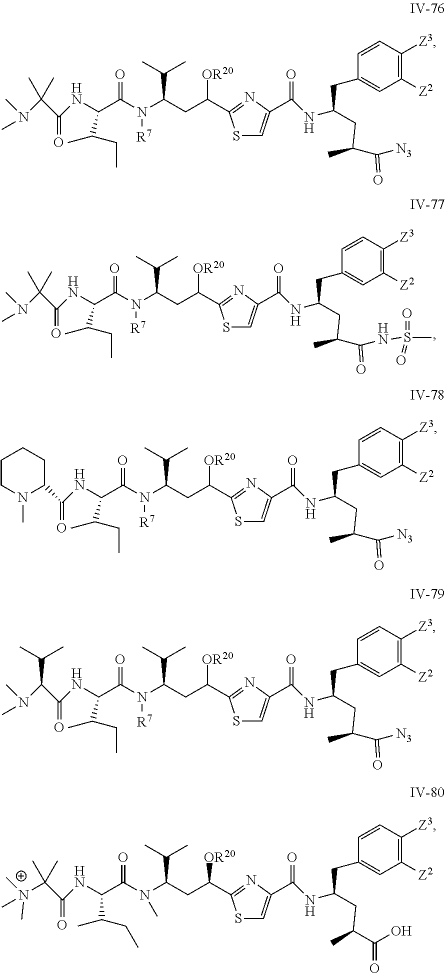 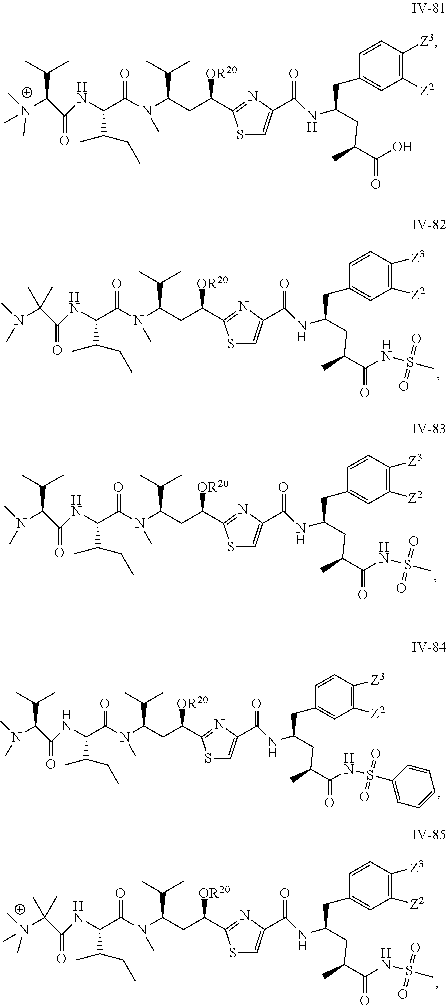 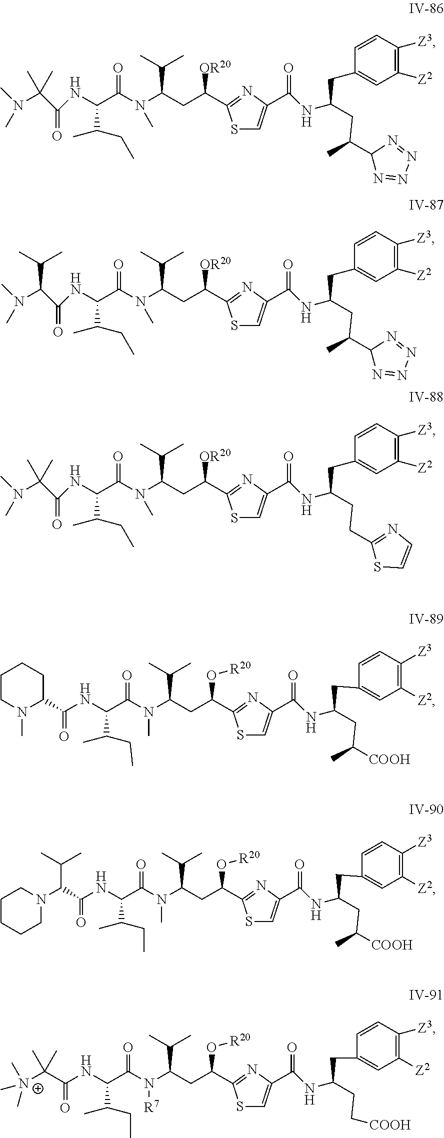 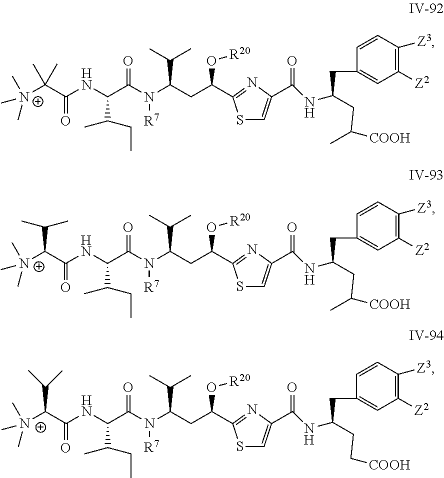
wherein R²⁰ is H; C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); or 1-8 carbon atoms of carboxylate, ester, ether, or amide; or 1˜8 amino acids; or polyethyleneoxy unit of formula (OCH₂CH₂)ₚ or (OCH₂CH(CH₃))ₚ, wherein p is an integer from 0 to about 1000; or R²⁰ is absent and the oxygen forms a ketone, or combination above thereof, Z³ and Z³ are independently H, OH, NH₂, O, NH, COOH, COO, C(O), C(O), C(O)NH, C(O)NH₂, R¹⁸, OCH₂OP(O)(OR¹⁸)₂, OC(O)OP(O)(OR¹⁸)₂, OPO(OR¹⁸)₂, NHPO(OR¹⁸)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁹ is H, OH, NH₂, OSO₂(OR¹⁸), XCH₂OP(O)(OR¹⁸)₂, XPO(OR¹⁸)₂, XC(O)OP(O)(OR¹⁸)₂, XC(O)R¹⁸, XC(O)NHR¹⁸, C₁˜C₈alkyl or carboylate; C₂˜C₈alkenyl, alkynyl, alkylcycloalkyl, heterocycloalkyl; C₃˜C₈ aryl or alkylcarbonyl; or pharmaceutical salts; X is O, S, NH, NHNH, or CH₂; R⁷ is defined the same above; wherein the linkage sites, “ [symbol] ” in formula IV-01-IV-94 are omitted here but they are followed the same positions indicated in Formula (IV) of claim 42;
(b). the calicheamicins and their related enediyne antibiotics having the following formula:
wherein the calicheamicin, geldanamycin, maytansinoid, eribulin, and spliceostatin, have the following formula respectively: 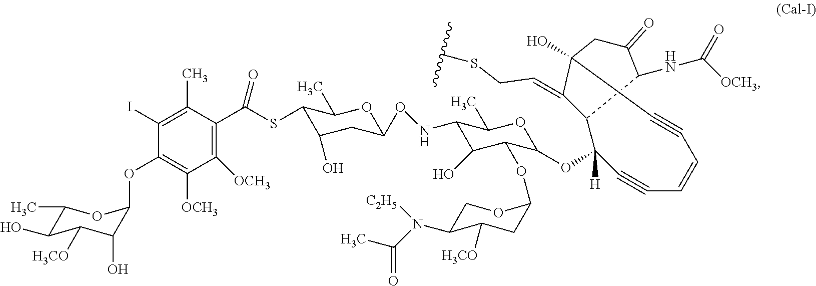 [SMILES: *SC/C=C1\C2[C@@H](OC3OC(C)C(NOC4CC(O)C(SC(=O)c5c(C)c(I)c(OC6OC(C)C(O)C(OC)C6O)c(OC)c5OC)C(C)O4)C(O)C3OC3CC(OC)C(N(CC)C(C)=O)CO3)C#C/C=C\C#C[C@]1(O)CC(=O)[C@@H]2NC(=O)OC]
or derivatives with one or more isotopes, or a pharmaceutically acceptable salt, hydrates, or hydrated salt; or a polymorphic crystalline structure; or an optical isomer, racemate, diastereomer or enantiomer thereof,
wherein [symbol] is the site linked to L₁ or L₂;
(c). the geldanamycin having the following formula: 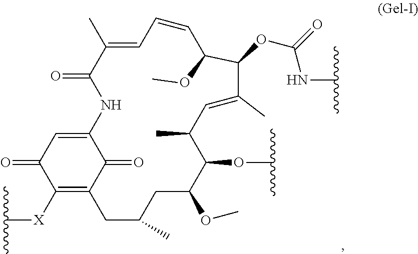
wherein X is O, NH, CH₂, [symbol] is the link site linked to L₁ or L₂;
(d). the Maytansines or their derivatives maytansinoids having the following formula: 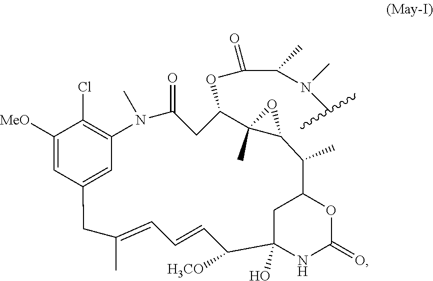 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)CC(OC(=O)N2)[C@@H](C)[C@H]2O[C@]12C]
or derivatives with one or more isotopes, or a pharmaceutically acceptable salt, hydrates, or hydrated salt; or a polymorphic crystalline structure; or an optical isomer, racemate, diastereomer or enantiomer thereof; wherein [symbol] is the site inked to L₁ or L₂;
(e). the camptothecin (CPTs) and its derivatives having the following formula: 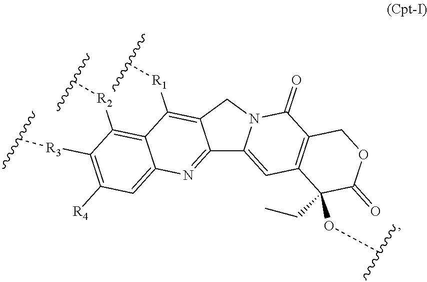
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R₁, R₂ and R₄ are independently selected from H, F, Cl, Br, CN, NO₂, C₁˜C₈ alkyl; O—C₁˜C₈alkyl; NH—C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, carbonate, urea, or carbamate; R₃ is H, OH, NH₂, C₁˜C₈ alkyl; O—C₁˜C₈ alkyl; NH—C₁˜C₈ alkyl; C₂-C₈ of heteroalkyl, alkylcycloalkyl, heterocycloalkyl; or 2-8 carbon atoms of esters, ether, amide, carbonate, urea, or carbamate; or R₁R₂, R₂R₃ and R₃R₄ independently form a 5-7 membered carbocyclic, heterocyclic, heterocycloalkyl, aromatic or heteroaromatic ring system; [symbol] is the site in the molecule that can be linked to L₁ or L₂;
the camptothecin (CPTs) and its derivatives of the formula (Cpt-I) have the following formula: 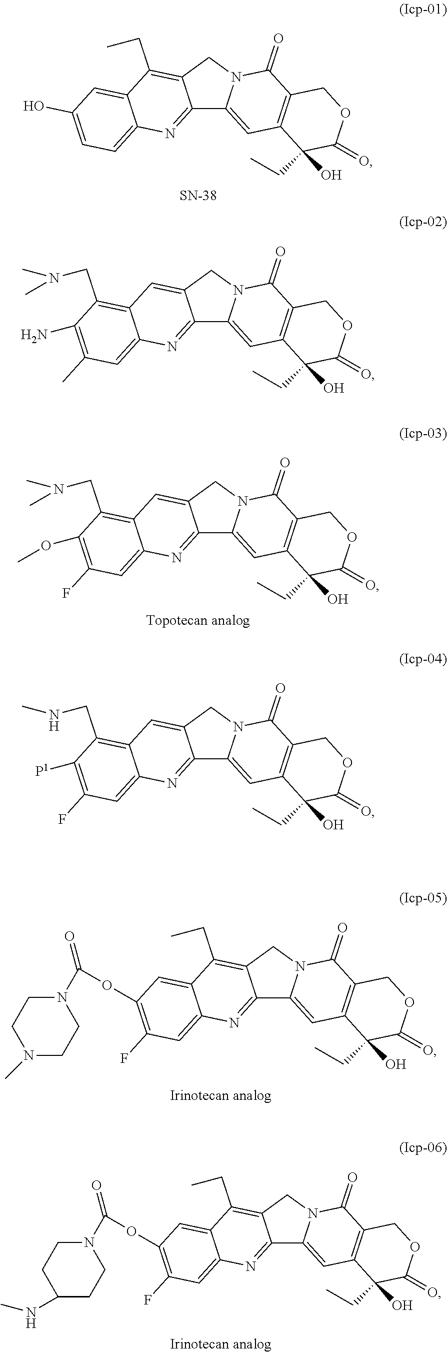 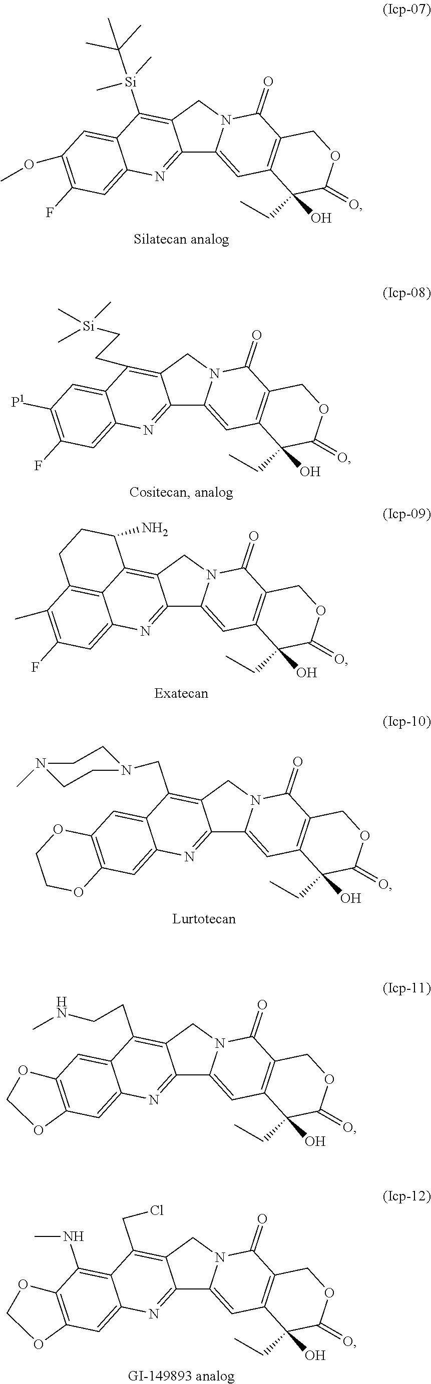 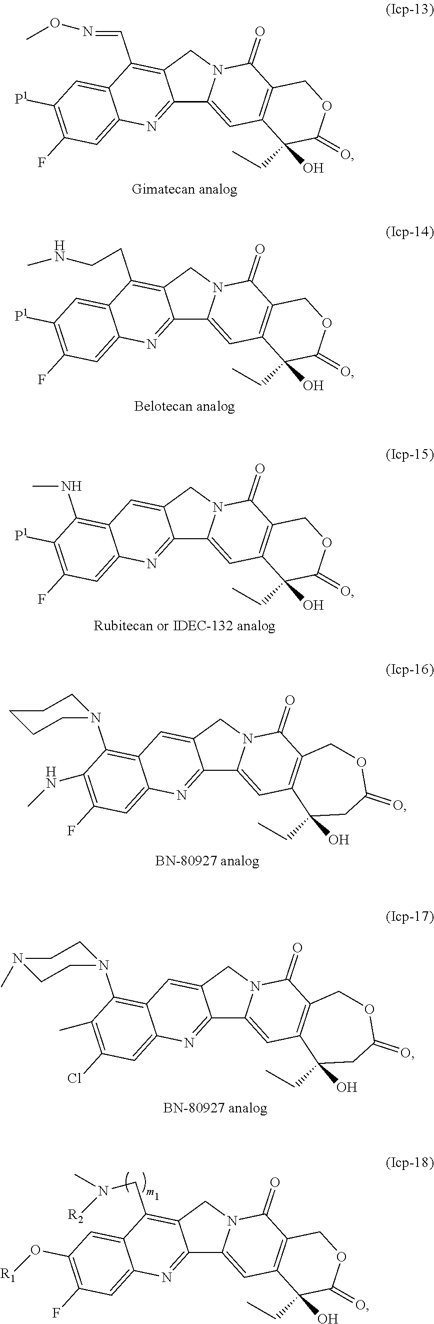 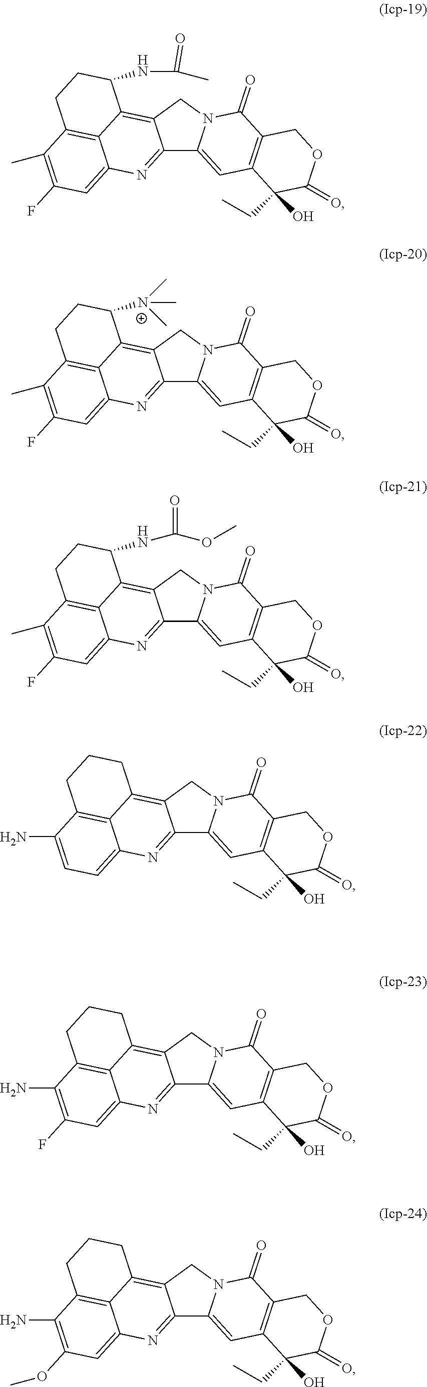 [SMILES: CC[C@@]1(O)C(=O)OCc2c1cc1n(c2=O)Cc2c-1nc1cc(F)c(C)c3c1c2[C@@H](NC(C)=O)CC3 ; CC[C@@]1(O)C(=O)OCc2c1cc1n(c2=O)Cc2c-1nc1cc(F)c(C)c3c1c2[C@@H]([N+](C)(C)C)CC3 ; CC[C@@]1(O)C(=O)OCc2c1cc1n(c2=O)Cc2c-1nc1cc(F)c(C)c3c1c2[C@@H](NC(=O)OC)CC3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1cc(F)c(N)c3c1c2CCC3 ; CC[C@@]1(O)C(=O)OCc2c1cc1n(c2=O)Cc2c-1nc1cc(OC)c(N)c3c1c2CCC3]
or an isotope of one or more chemical elements, or pharmaceutically acceptable salts, hydrates, or hydrated salts; or the polymorphic crystalline structures of these compounds; or the optical isomers, racemates, diastereomers or enantiomers; P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ of linear or branched alkyl), C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R₁ and R₂ are independently selected from H; O—C₁˜C₈ alkyl; C₂-C₈ of heteroalkyl, alkylcycloalkyl, heterocycloalkyl; C₃-C₈ of aryl, Ar-alkyl; wherein the linkage sites, “ [symbol] ” in the above CPTs structures (Icp-01-Icp-24) are omitted here but they are defined the same positions as indicated in formula (Cpt-I);
(f). the Combretastatin and its derivatives having the following formula: 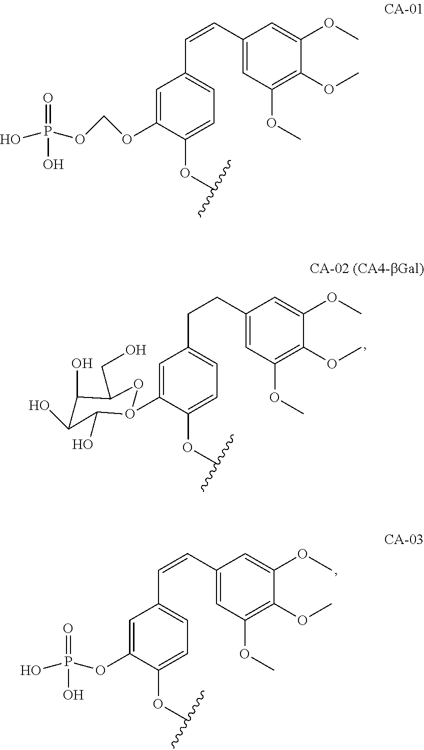
or derivatives with one or more isotopes, or a pharmaceutically acceptable salt, hydrates, or hydrated salt; or a polymorphic crystalline structure; or an optical isomer, racemate, diastereomer or enantiomer thereof; wherein [symbol] is the site linked to L₁ or L₂;
(g). the Taxane and its derivatives having the following formula: 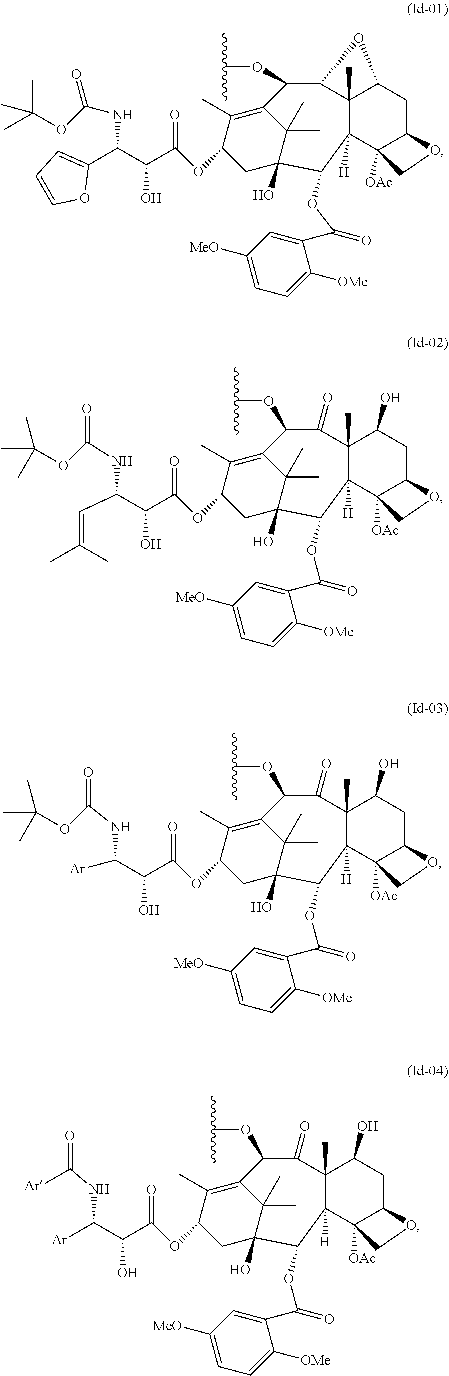
or derivatives with one or more isotopes, or a pharmaceutically acceptable salt, hydrates, or hydrated salt; or a polymorphic crystalline structure; or an optical isomer, racemate, diastereomer or enantiomer thereof; wherein [symbol] is the site linked to L₁ or L₂; Ar and Ar′ are independently aryl or heteroaryl;
(h). the anthracycline and its derivatives having the following formula: 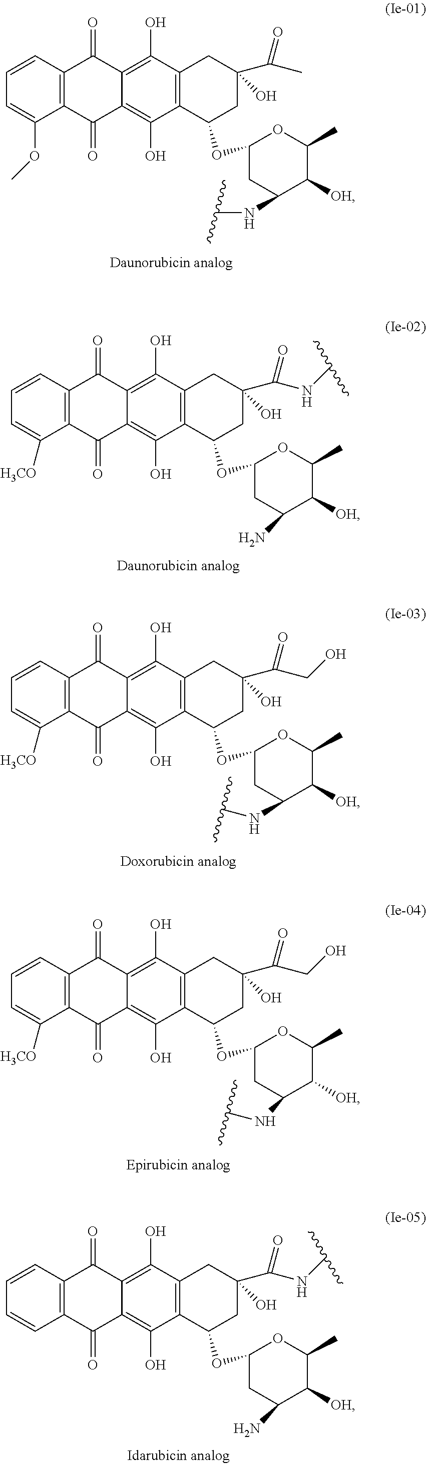 [SMILES: *N[C@H]1C[C@H](O[C@H]2C[C@](O)(C(C)=O)Cc3c(O)c4c(c(O)c32)C(=O)c2c(OC)cccc2C4=O)O[C@@H](C)[C@H]1O ; *NC(=O)[C@]1(O)Cc2c(O)c3c(c(O)c2[C@@H](O[C@H]2C[C@H](N)[C@H](O)[C@H](C)O2)C1)C(=O)c1c(OC)cccc1C3=O ; *N[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O ; *N[C@H]1C[C@H](O[C@H]2C[C@](O)(C(=O)CO)Cc3c(O)c4c(c(O)c32)C(=O)c2c(OC)cccc2C4=O)O[C@@H](C)[C@@H]1O ; *NC(=O)[C@]1(O)Cc2c(O)c3c(c(O)c2[C@@H](O[C@H]2C[C@H](N)[C@H](O)[C@H](C)O2)C1)C(=O)c1ccccc1C3=O] 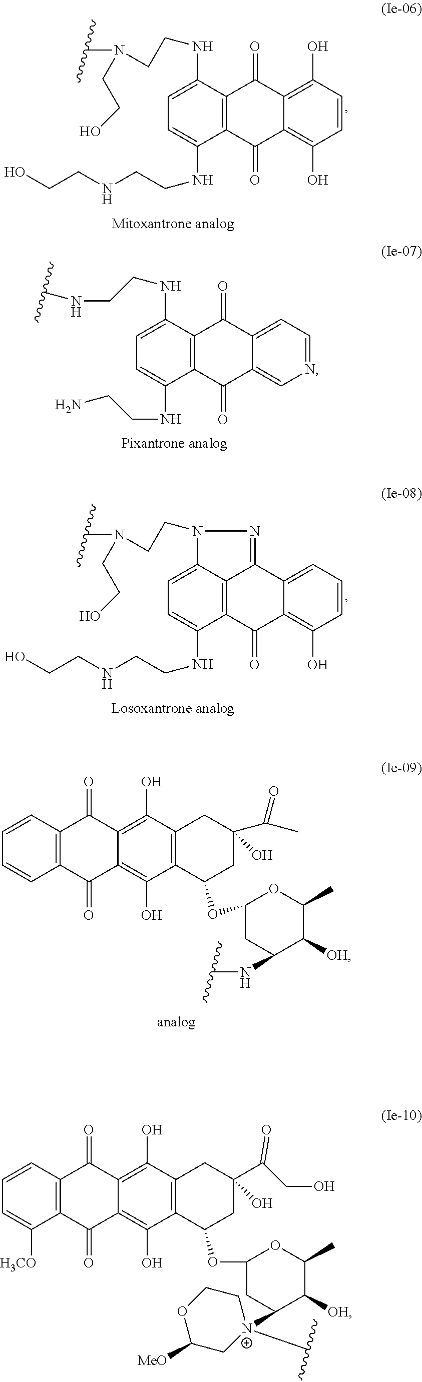 [SMILES: *N(CCO)CCNc1ccc(NCCNCCO)c2c1C(=O)c1c(O)ccc(O)c1C2=O ; *NCCNc1ccc(NCCN)c2c1C(=O)c1ccncc1C2=O ; *N(CCO)CCn1nc2c3c(c(NCCNCCO)ccc31)C(=O)c1c(O)cccc1-2 ; *N[C@H]1C[C@H](O[C@H]2C[C@](O)(C(C)=O)Cc3c(O)c4c(c(O)c32)C(=O)c2ccccc2C4=O)O[C@@H](C)[C@H]1O ; *[N+]1([C@H]2CC(O[C@H]3C[C@](O)(C(=O)CO)Cc4c(O)c5c(c(O)c43)C(=O)c3c(OC)cccc3C5=O)O[C@@H](C)[C@H]2O)CCO[C@H](OC)C1] 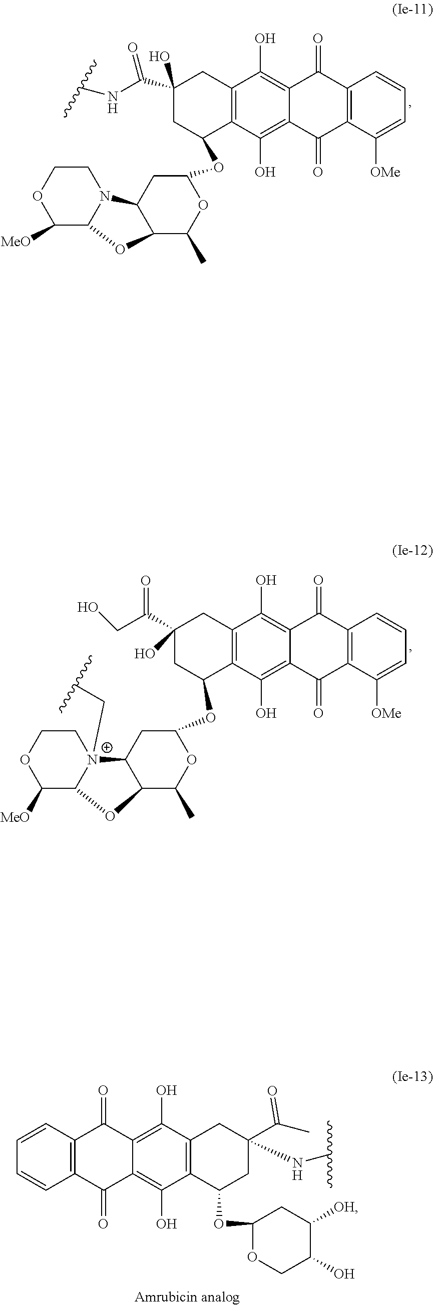
or derivatives with one or more isotopes, or a pharmaceutically acceptable salt, hydrates, or hydrated salt; or a polymorphic crystalline structure; or an optical isomer, racemate, diastereomer or enantiomer thereof; wherein [symbol] is the site linked to L₁ or L₂;
(i). the Vinca alkaloid and its derivatives having the following formula: 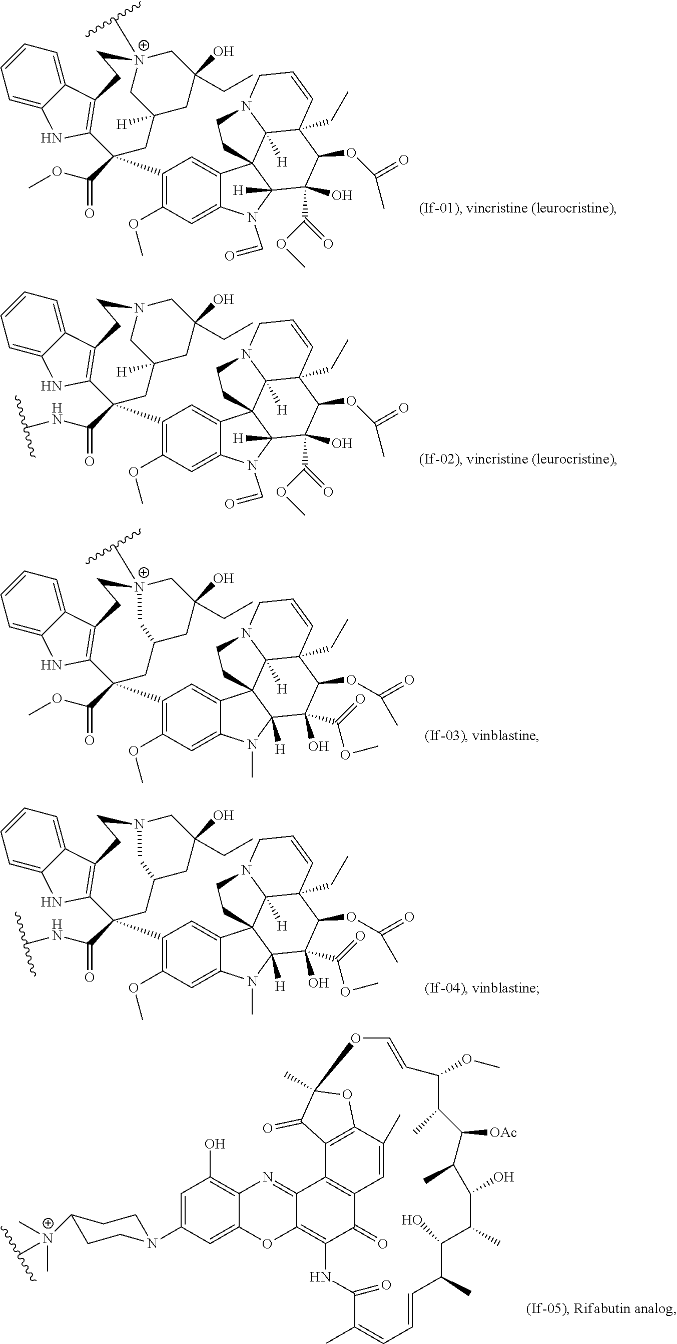 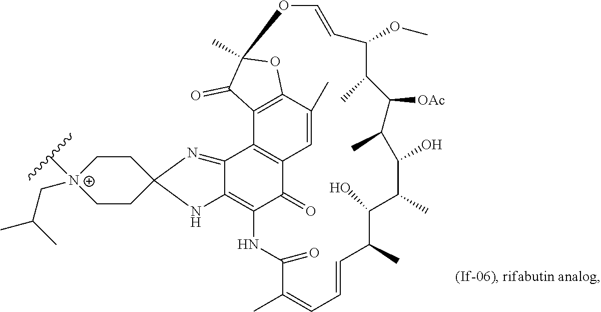 [SMILES: *[N+]1(CC(C)C)CCC2(CC1)N=C1C(=C3NC(=O)/C(C)=C\C=C\[C@H](C)[C@H](O)[C@@H](C)[C@@H](O)[C@@H](C)[C@H](OC(C)=O)[C@H](C)[C@@H](OC)/C=C/O[C@@]4(C)Oc5c(C)cc(c1c5C4=O)C3=O)N2]
or derivatives with one or more isotopes, or a pharmaceutically acceptable salt, hydrates, or hydrated salt; or a polymorphic crystalline structure; or an optical isomer, racemate, diastereomer or enantiomer thereof, wherein [symbol] is the site linked to L₁ or L₂;
(j). the dolastatin, auristatin and their derivatives having the following formula:
(Ih-01), (Ih-02), (Ih-03), (Ih-04), (Ih-05), (Ih-06), (Ih-07), (Ih-08), (Ih-09), (Ih-10), and (Ih-11): 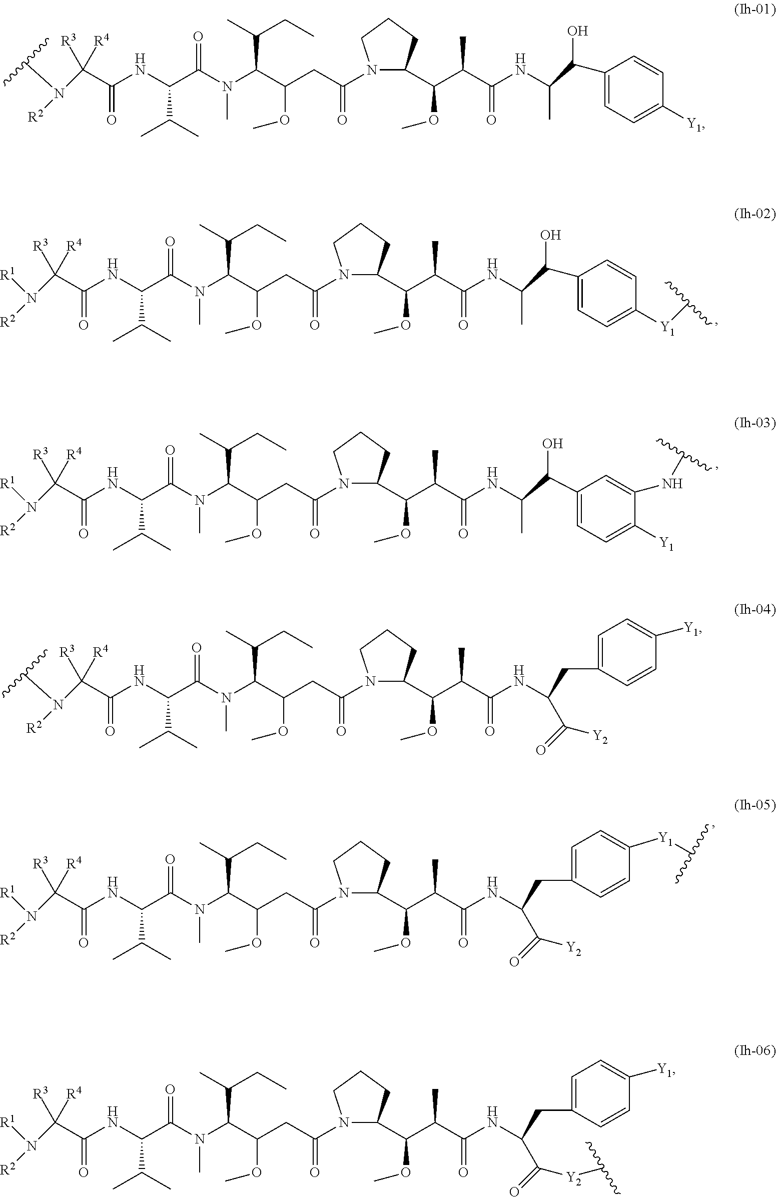 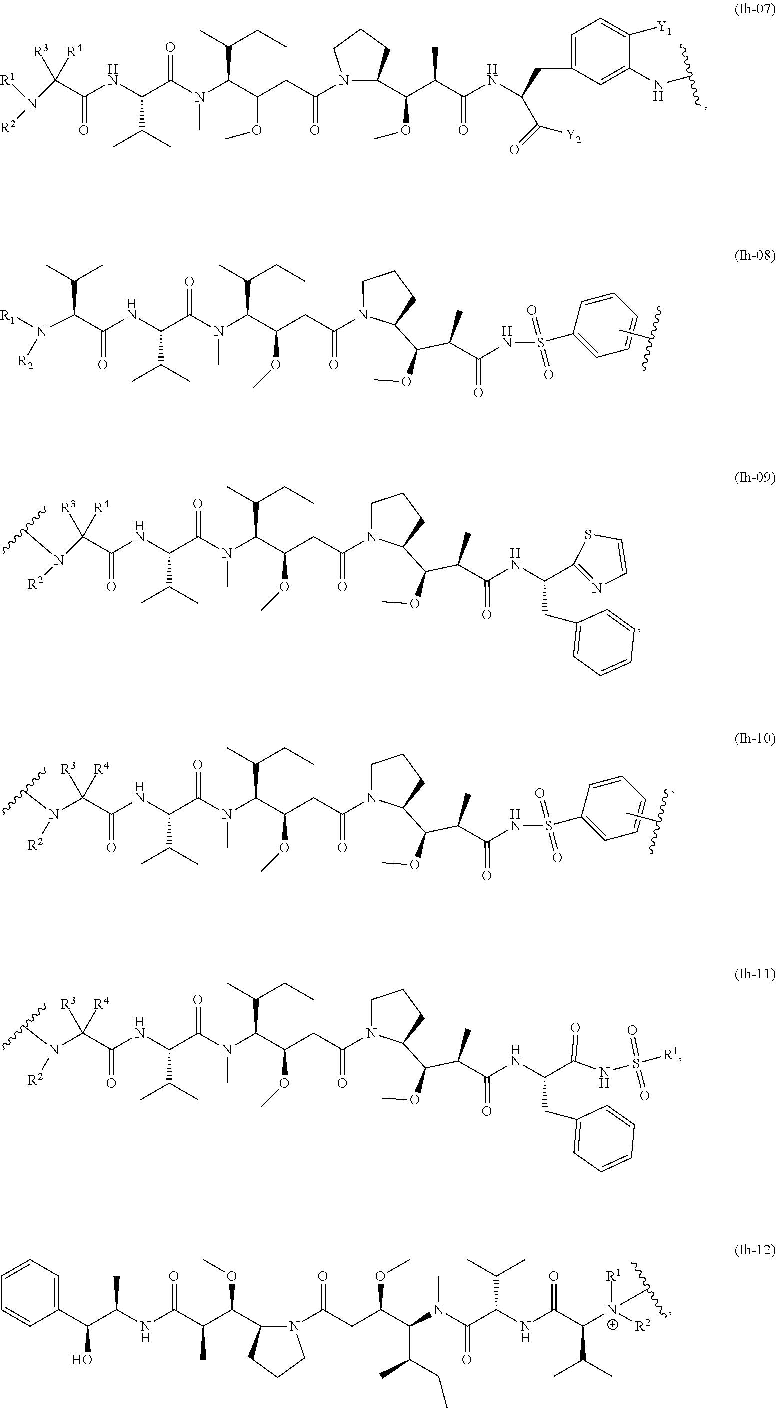 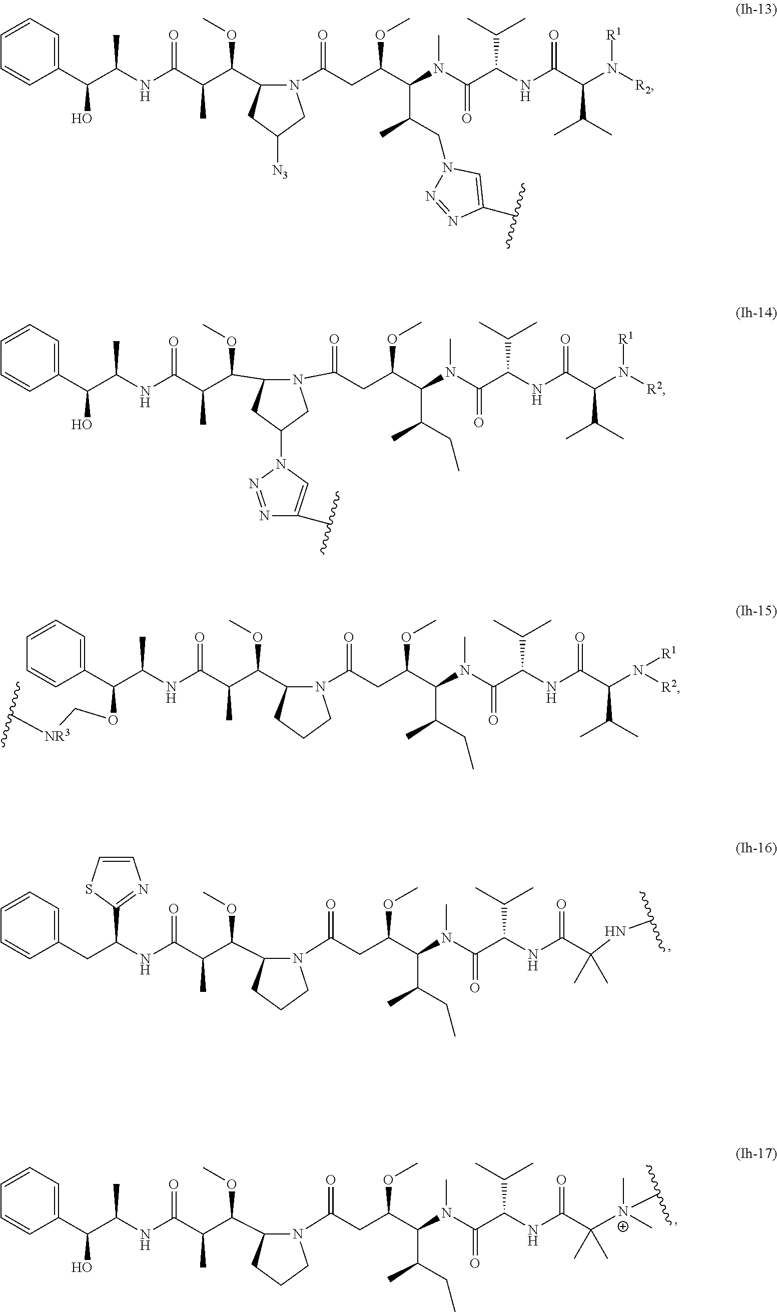
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R¹, R², R³, R⁴ and R⁵ are independently H; C₁-C₈ linear or branched alkyl, aryl, heteroaryl, heteroalkyl, alkylcycloalkyl, ester, ether, amide, amine, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 1000; two Rs: R¹R², R²R³, R¹R³ or R³R⁴ together can form 3˜8 member cyclic ring of alkyl, aryl, heteroaryl, heteroalkyl, or alkylcycloalkyl group; Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ” (that links to L₁ and/or L₂ independently); or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different amino acids; p is 1-5000; R₁, R₂, R₃, R₄, R₅, R₅′, Z₁, Z₂, and n are defined the same above;
(k). the Hemiasterlin and its derivatives having the following formula: 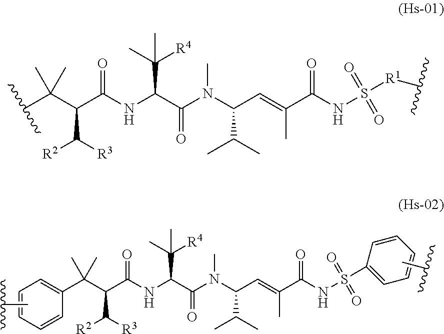
or derivatives with one or more isotopes, or a pharmaceutically acceptable salt, hydrates, or hydrated salt; or a polymorphic crystalline structure; or an optical isomer, racemate, diastereomer or enantiomer thereof, wherein [symbol] is the site linked to L₁ or L₂; wherein R¹, R², R³, R⁴ and R⁵ are independently H; C₁-C₈ linear or branched alkyl, aryl, heteroaryl, heteroalkyl, alkylcycloalkyl, ester, ether, amide, amine, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; or R²R³ can form 3˜8 member cyclic ring of alkyl, aryl, heteroaryl, heteroalkyl, or alkylcycloalkyl group;
(l). the Eribulin and its derivatives having the following formula: 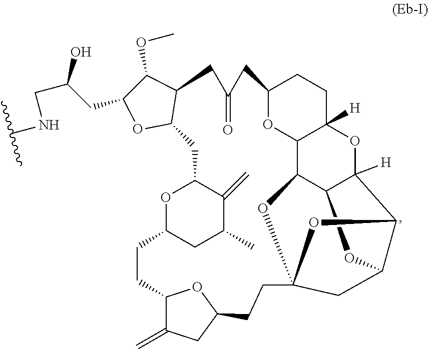 [SMILES: *NC[C@@H](O)C[C@H]1O[C@H]2C[C@H]3O[C@@H](CC[C@@H]4O[C@@H](CC[C@@]56C[C@H]7O[C@@H]8C(O[C@H]9CC[C@H](CC(=O)C[C@@H]2[C@H]1OC)OC9[C@@H]8O5)[C@H]7O6)CC4=C)C[C@@H](C)C3=C]
or derivatives with one or more isotopes, or a pharmaceutically acceptable salt, hydrates, or hydrated salt; or a polymorphic crystalline structure; or an optical isomer, racemate, diastereomer or enantiomer thereof; wherein [symbol] is the site linked to L₁ or L₂;
(m). the Inhibitor of nicotinamide phosphoribosyltransferases (NAMPT) and their derivatives having the following formula: NP01, NP02, NP03, NP04, NP05, NP06, NP07, NP08, and NP09: 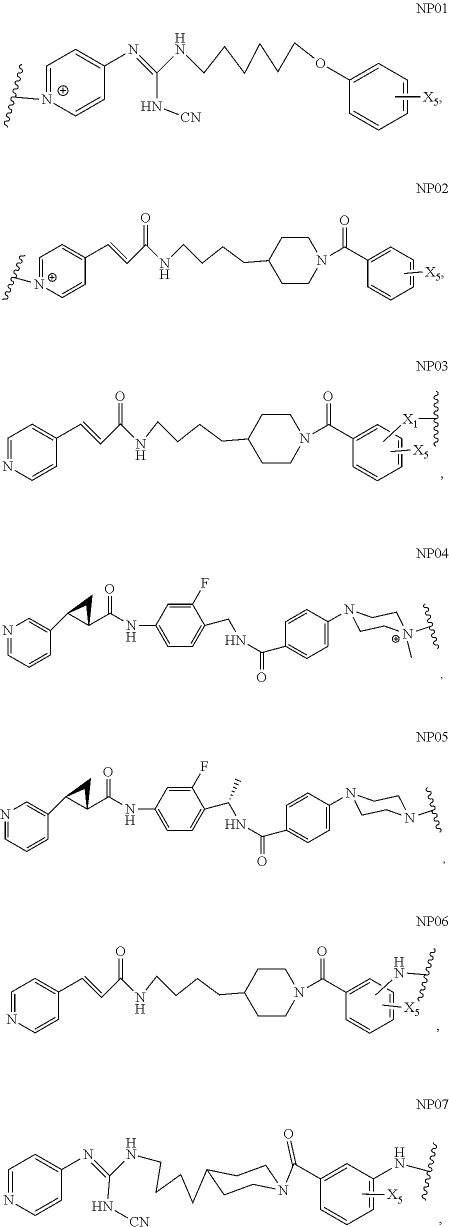 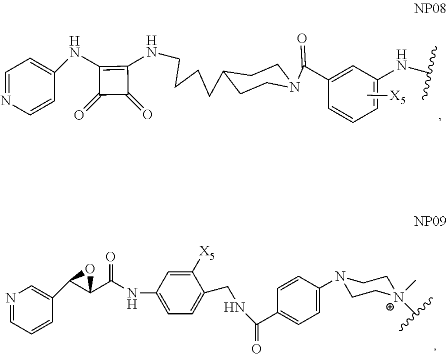
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR₁, OCOR₁, NHCOR₁;
(n). the benzodiazepine dimer and its derivatives having the following formula: PB01, PB02, PB03, PB04, PB05, PB06, PB07, PB08, PB09, PB10, PB11, PB12, PB13, PB14, PB15, PB16, PB17, PB18, PB19, PB20: 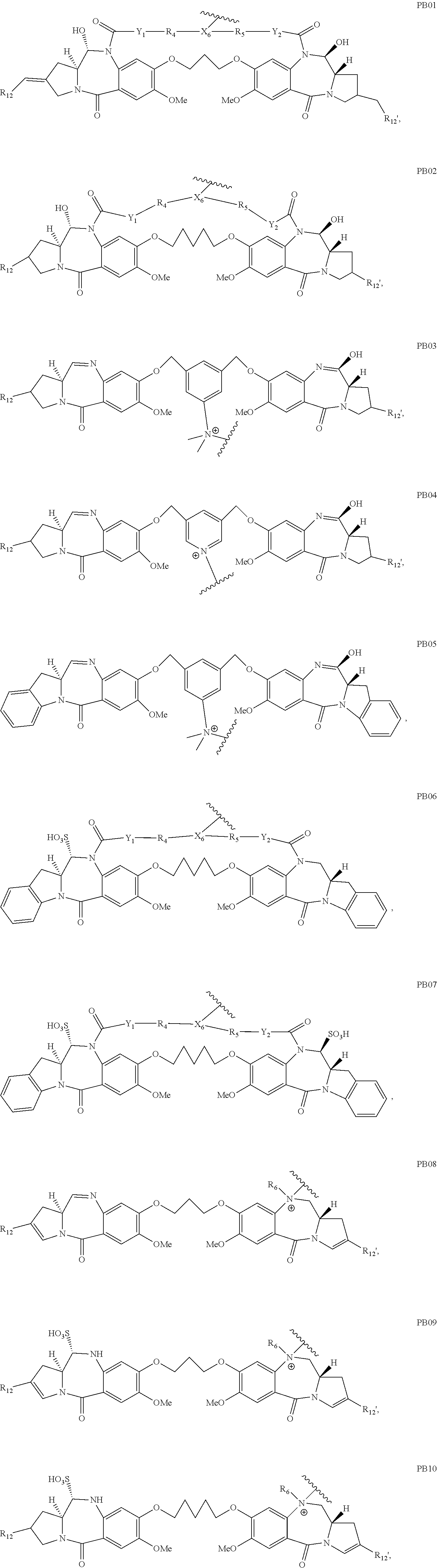 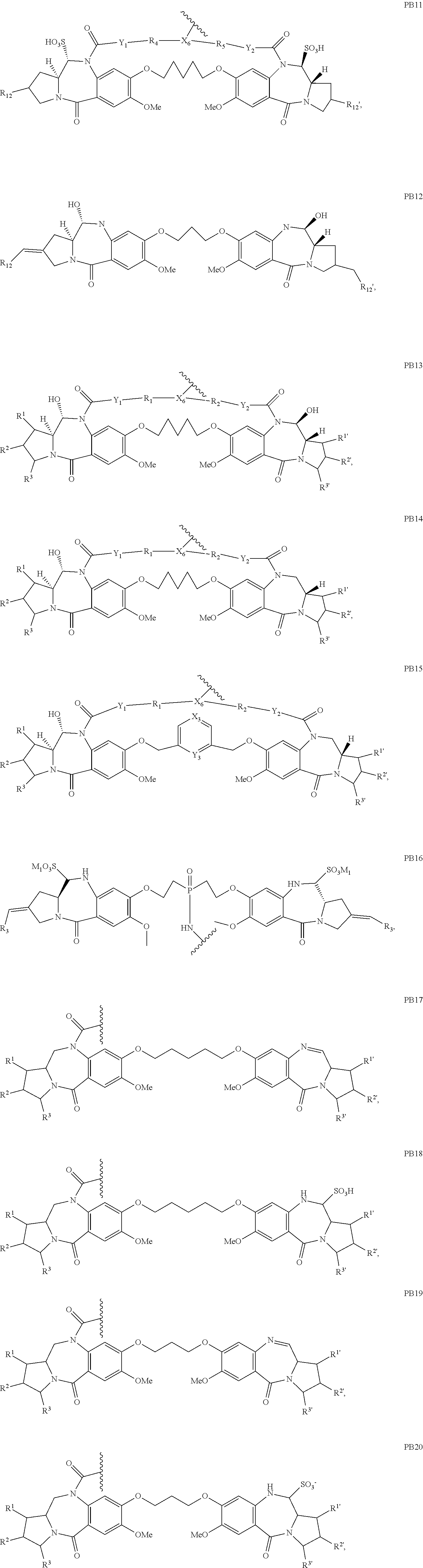
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁, Y₂, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R¹, R², R³, R^{1′}, R^{2′}, and R^{3′} are independently H; F; Cl; ═O; ═S; ═CH₂; ═CH—R₁, OH; SH; C₁-C₈ linear or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amines (NHR₅, NR₅R_{5′}), heterocycloalkyl, or acyloxylamines (—C(O)NHOH, —ONHC(O)R₅); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000; two Rs: R¹R², R²R³, R¹R³, R^{1′}R^{2′}, R^{2′}R^{3′}, or R^{1′}R^{3′} can independently form 3˜8 member cyclic ring of alkyl, aryl, heteroaryl, heteroalkyl, or alkylcycloalkyl group; X₃ and Y₃ are independently N, NH, CH₂ or CR₅, or one of X₃ and Y₃ can be absent; wherein R₁, and R₂ are C₁-C₈ linear or branched alkyl, heteroalkyl; C₃-C₈aryl, heteroaryl, alkylcycloalkyl, acyloxyl, alkylaryl, alkylaryloxyl, alkylarylamino, alkylarylthiol; or 1-6 the same or different sequence of amino acid/peptides (Aa)ᵣ, r=1-6; wherein R₄, R₅, R₅′,R₆, R₁₂ and R₁₂′ are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M1, or O-glycoside (glucoside, galactoside, mannoside, glucuronoside/glucuronide, alloside, fructoside, etc.), NH-glycoside, S-glycoside or CH₂-glycoside; Mi and M2 are independently H, Na, K, Ca, Mg, NH₄, NR₁R₂R₃; X₆ is CH, N, P(O)NH, P(O)NR₁, CHC(O)NH, C₃-C₈aryl, heteroaryl, alkylcycloalkyl, acyloxyl, alkylaryl, alkylaryloxyl, alkylarylamino, or an Aa (amino acid); “ [symbol] ” is defined the same above;
(o). the CC-1065 analog or doucarmycin analogs and their derivatives having the following formula: CC01, CC02, CC03, CC04, CC05, CC06 and CC07: 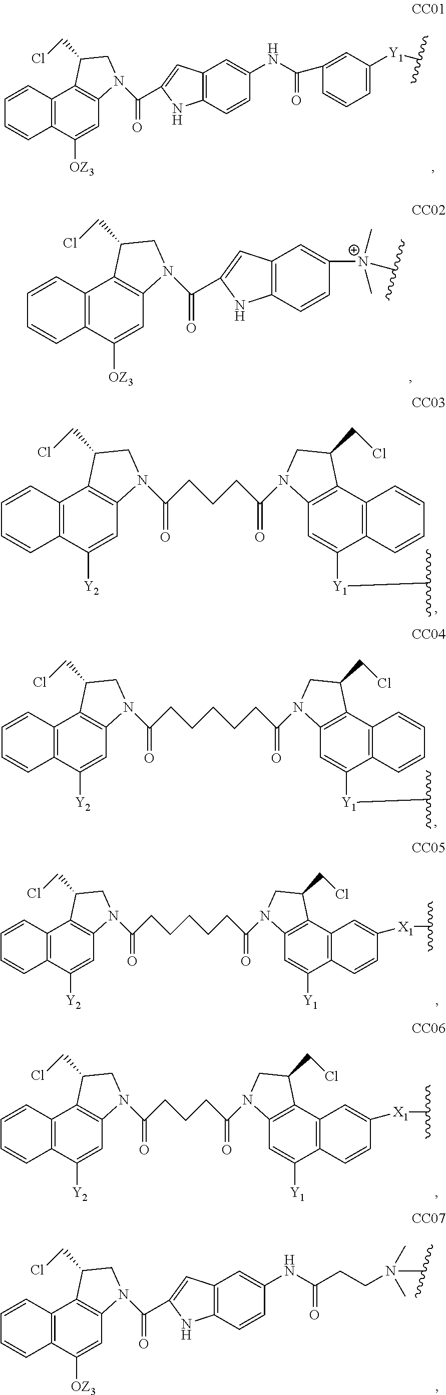
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are defined the same above;
(p). the amatoxin and its derivatives having the following formula: Am01, Am02, and Am03: 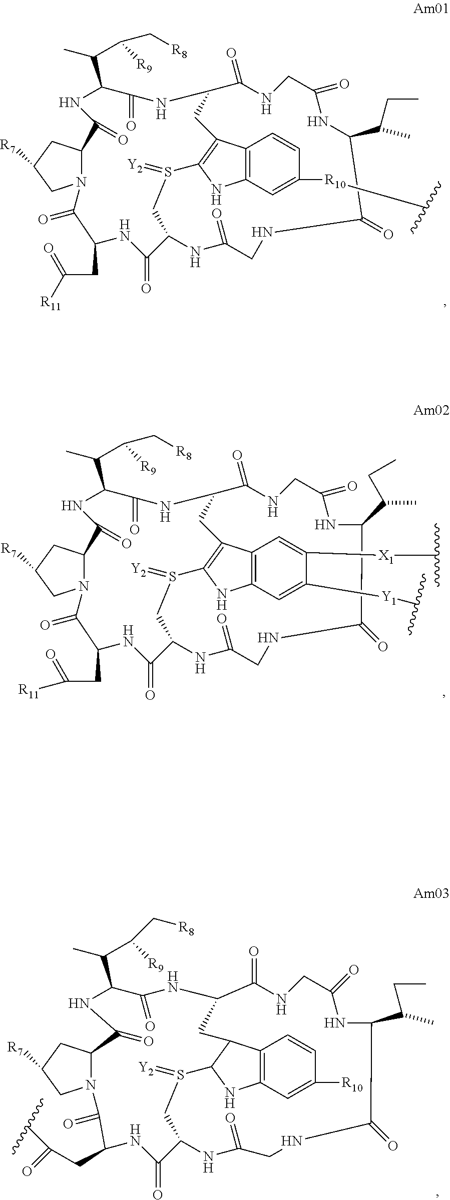
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NHNHC(O) and C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa)ᵣ is 1-8 aminoacids; n and m₁ are independently 1-20; p is 1-5000; R₁, R₂ and Ar, are the same defined; “ [symbol] ” is defined the same above;
(q). the spliceostatin, pladienolide and their derivatives having the following formula: 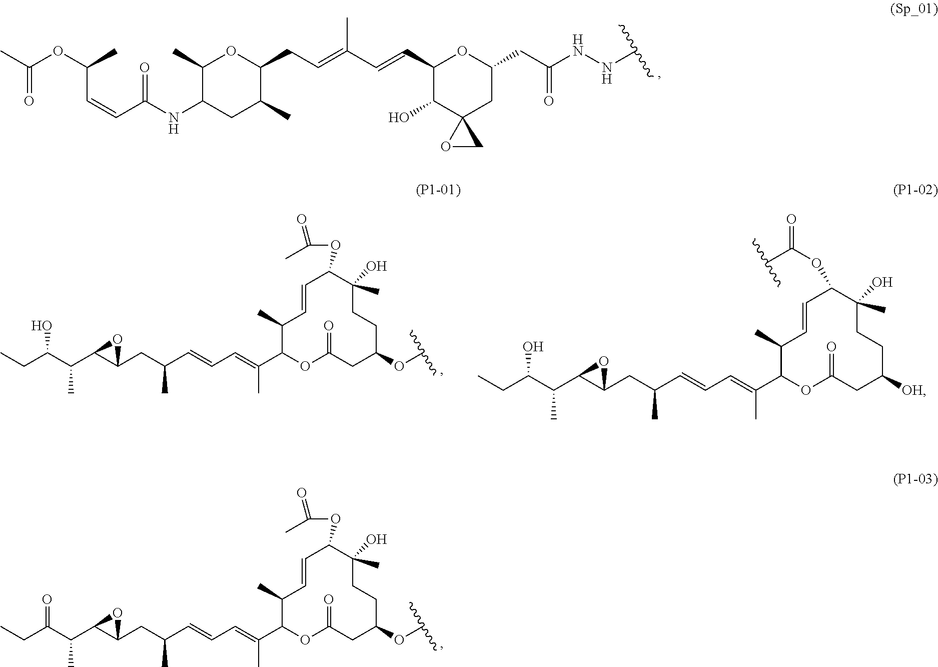 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1 ; *O[C@@H]1CC[C@@](C)(O)[C@@H](OC(C)=O)/C=C/[C@H](C)C(/C(C)=C/C=C/[C@@H](C)C[C@H]2O[C@@H]2[C@H](C)[C@@H](O)CC)OC(=O)C1 ; *C(=O)O[C@H]1/C=C/[C@H](C)C(/C(C)=C/C=C/[C@@H](C)C[C@H]2O[C@@H]2[C@H](C)[C@@H](O)CC)OC(=O)C[C@H](O)CC[C@@]1(C)O ; *O[C@@H]1CC[C@@](C)(O)[C@@H](OC(C)=O)/C=C/[C@H](C)C(/C(C)=C/C=C/[C@@H](C)C[C@H]2O[C@@H]2[C@H](C)C(=O)CC)OC(=O)C1]
or an isotope of a chemical element, or a pharmaceutically acceptable salt, hydrates, or hydrated salt; or a polymorphic crystalline structure; or an optical isomer, racemate, diastereomer or enantiomer thereof, wherein [symbol] is the link site linked to L₁ or L₂;
(r). the Protein kinase inhibitors and their derivatives having the following formula: PK01˜PK40: 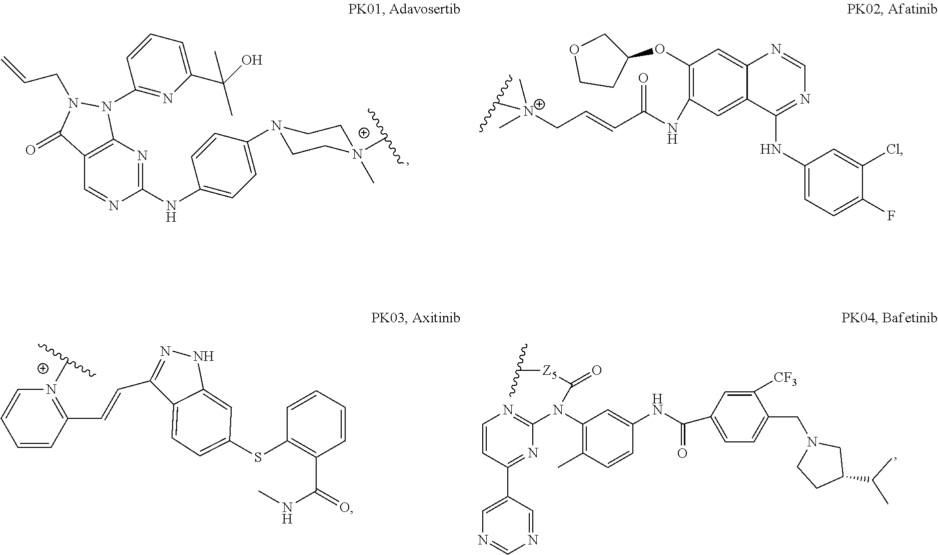 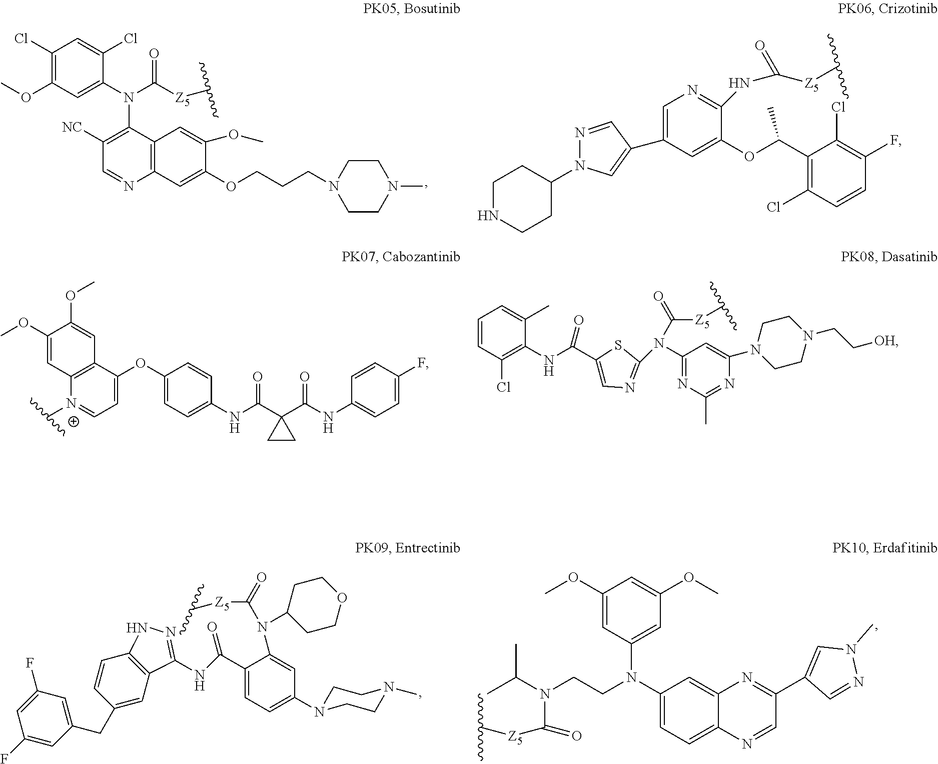 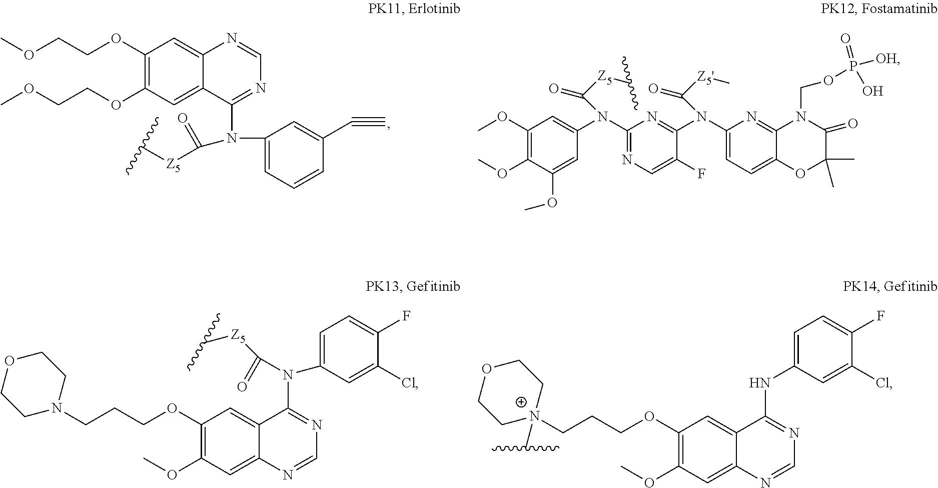 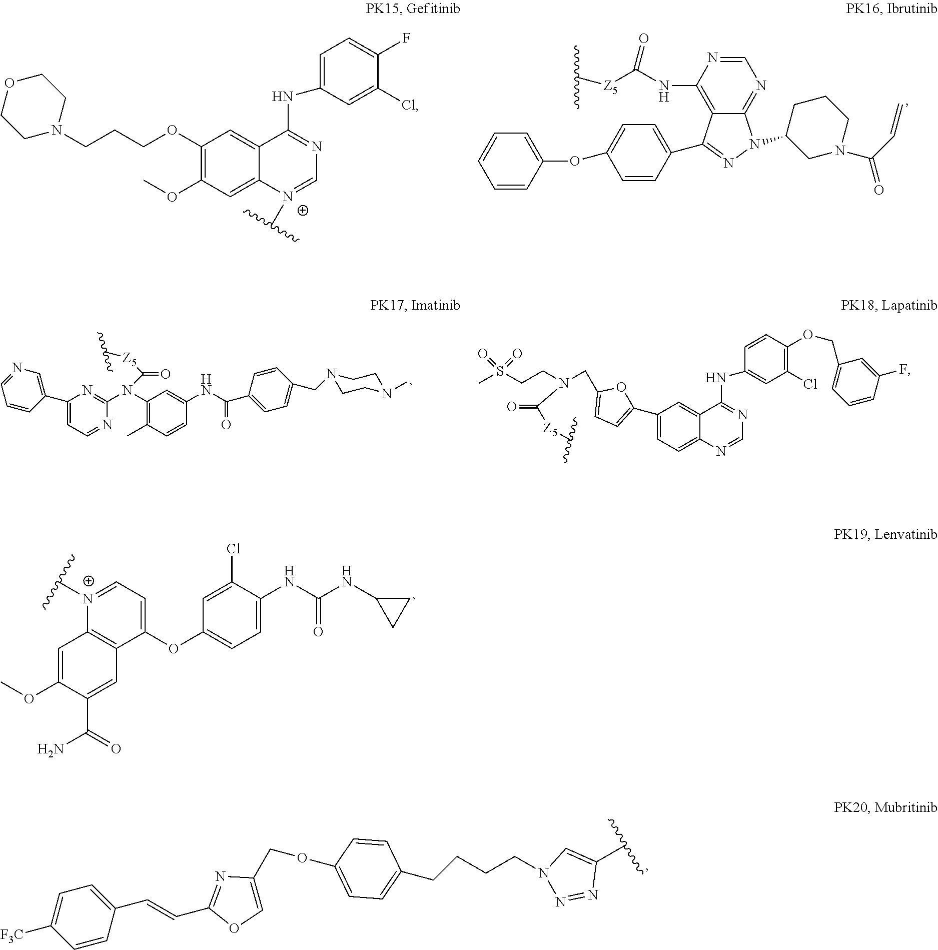 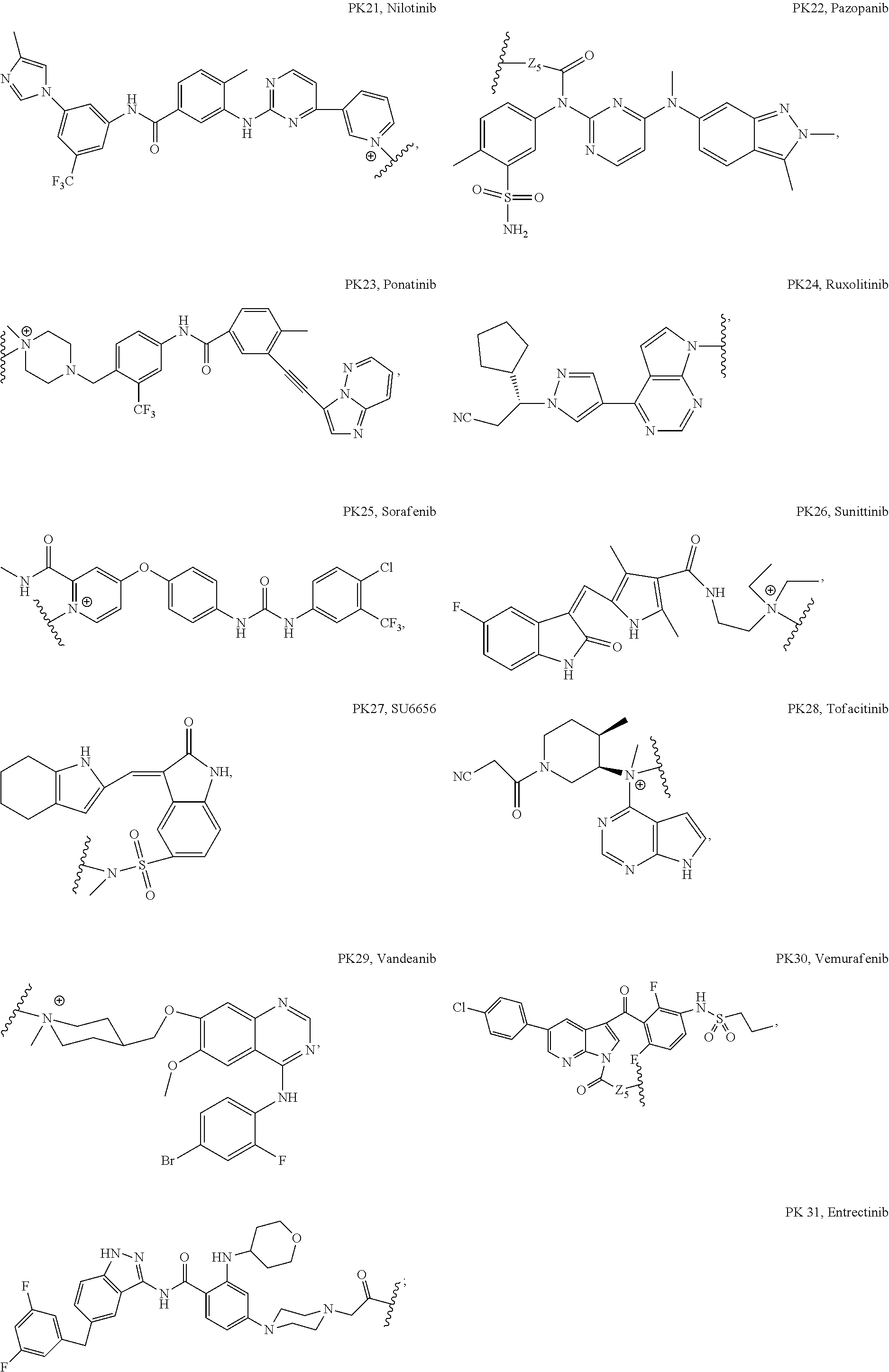 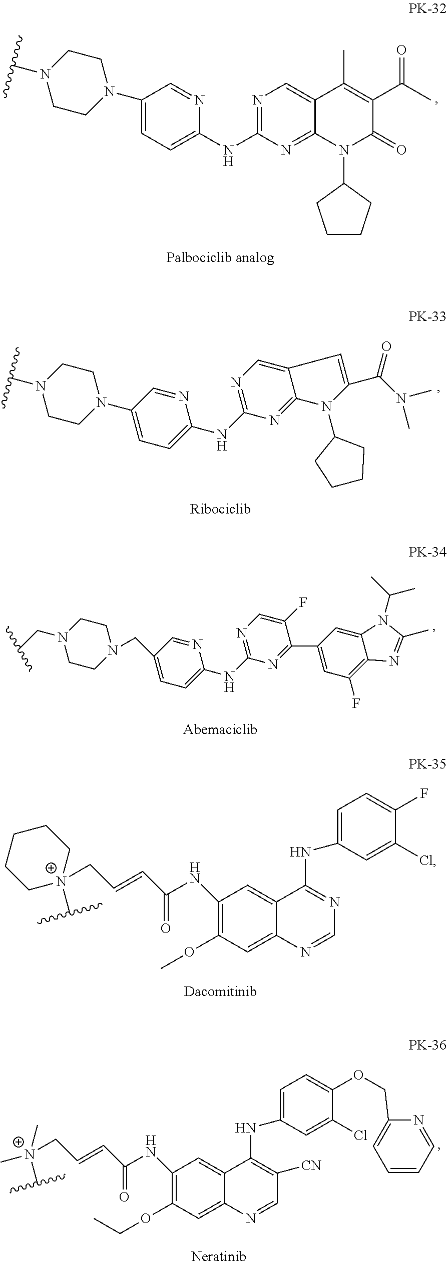 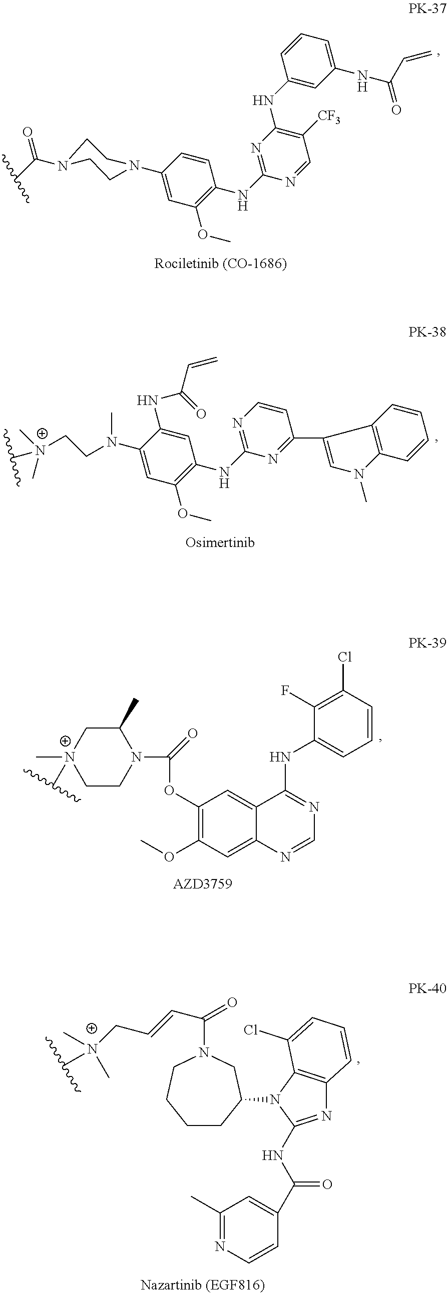
or an isotope of a chemical element, or a pharmaceutically acceptable salt, hydrates, or hydrated salt; or a polymorphic crystalline structure; or an optical isomer, racemate, diastereomer or enantiomer thereof, wherein Z₅ and Z₅′ are independently selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁, wherein [symbol] is the link site linked to L₁ or L₂;
(s). the MEK inhibitor and its derivatives having the following formula: 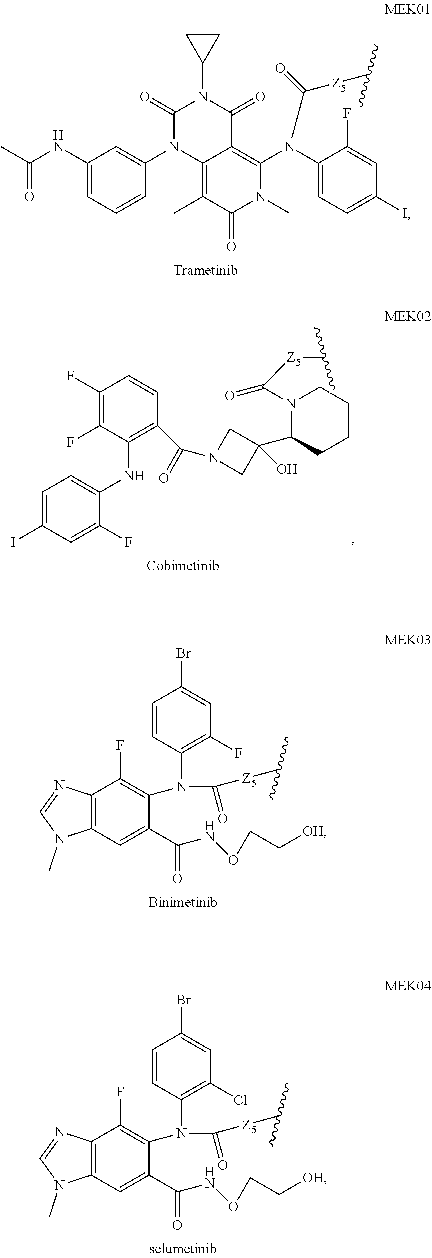
or an isotope of a chemical element, or a pharmaceutically acceptable salt, hydrates, or hydrated salt; or a polymorphic crystalline structure; or an optical isomer, racemate, diastereomer or enantiomer thereof, wherein Z₅ is selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁; wherein [symbol] is the site linked to L₁ or L₂;
(t). the proteinase inhibitor and its derivatives having the following formula: 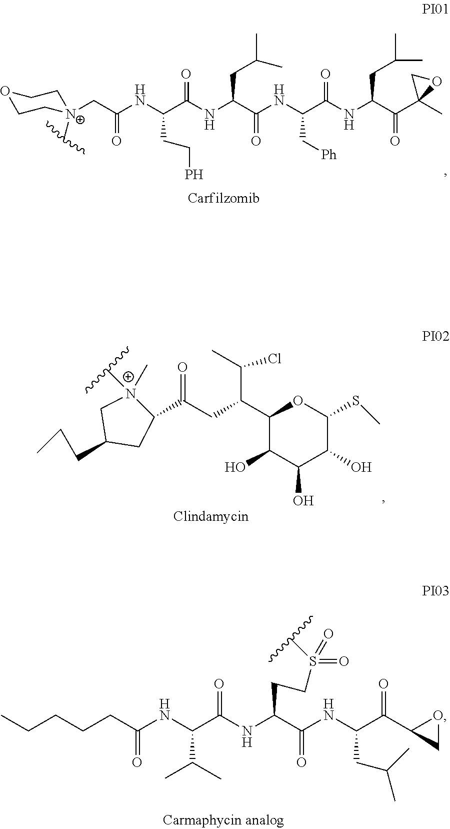
or an isotope of a chemical element, or a pharmaceutically acceptable salt, hydrates, or hydrated salt; or a polymorphic crystalline structure; or an optical isomer, racemate, diastereomer or enantiomer thereof, wherein [symbol] is the link site linked to L₁ or L₂;
(u). the immunotoxins that can be used as payloads for the conjugation are selected from Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, proaerolysin and topsalysin, the immunotoxins are constructed to the antibody via a bioengineering of protein fusions, or via through an amino acid of the immunotoxin having free amino, thiol or carboxyl acid group;
(v). the cell-binding molecule/ligand or a cell receptor agonist and their derivatives having the following formula: 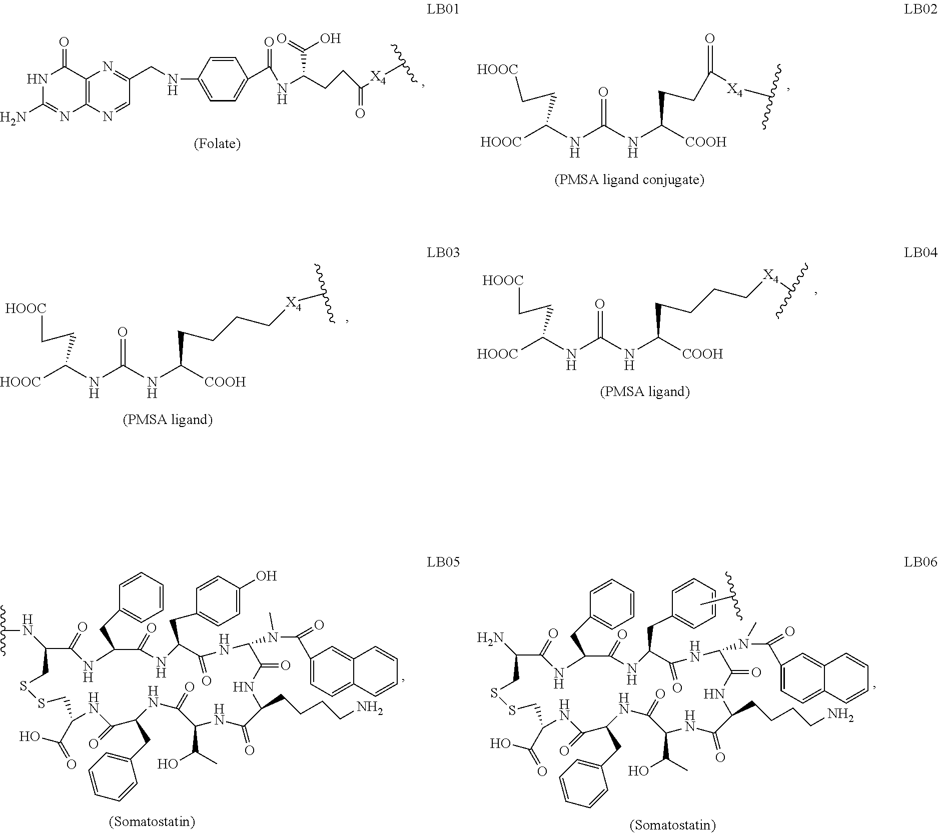 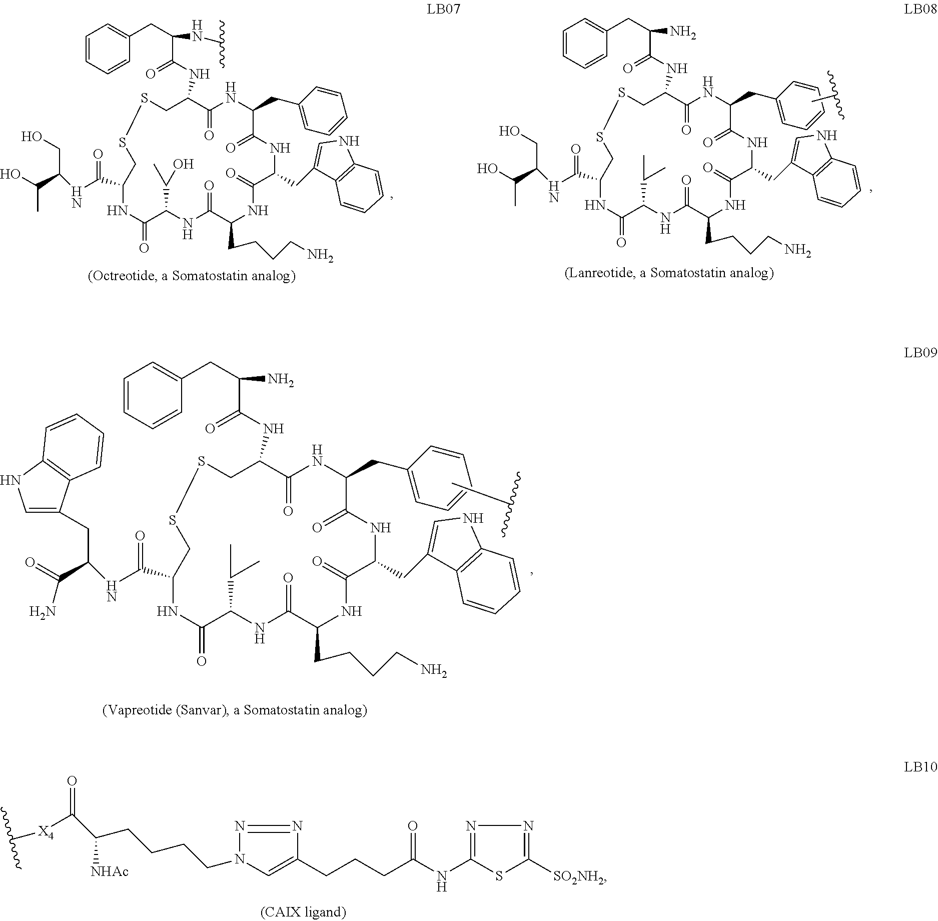 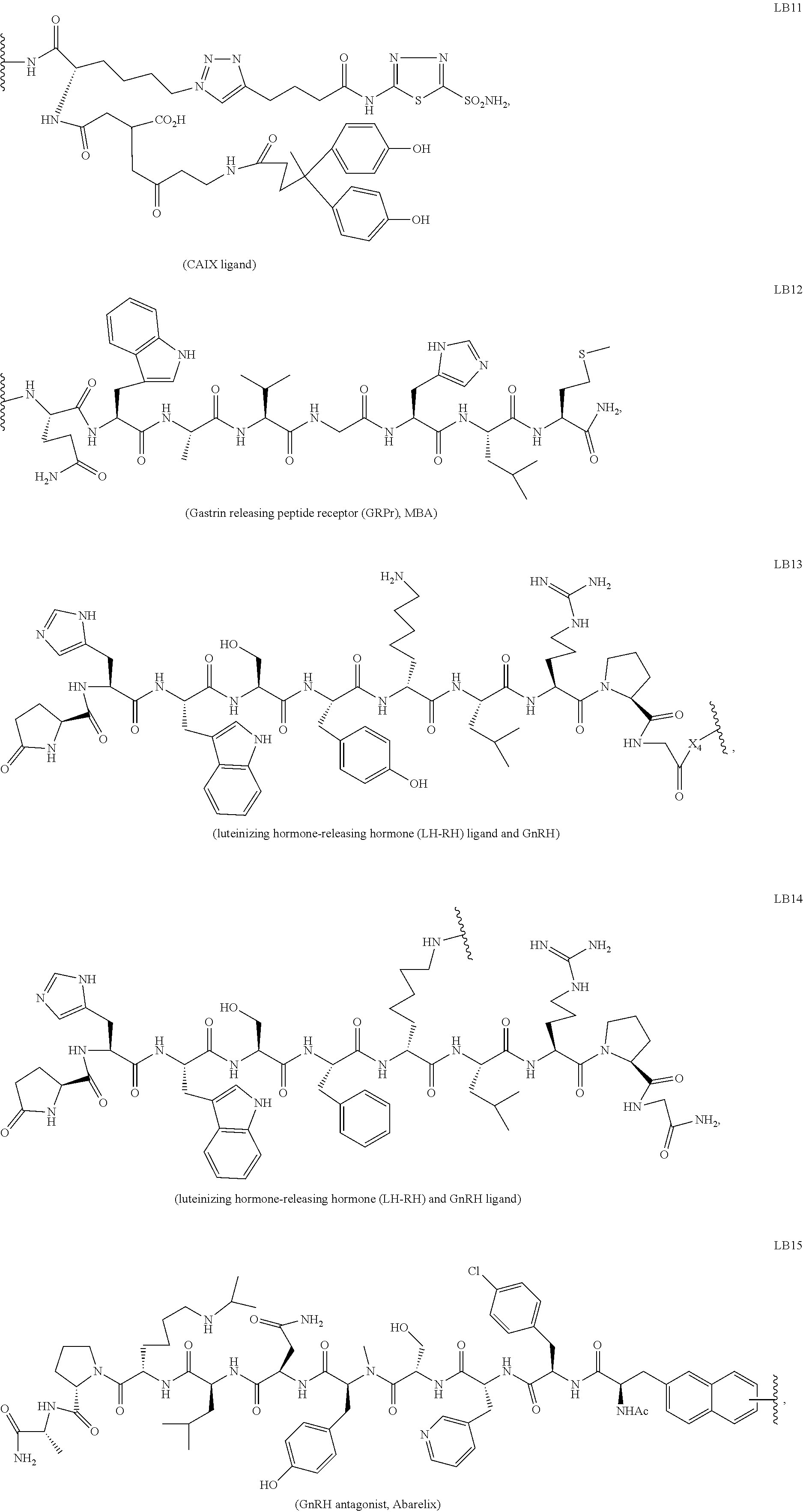 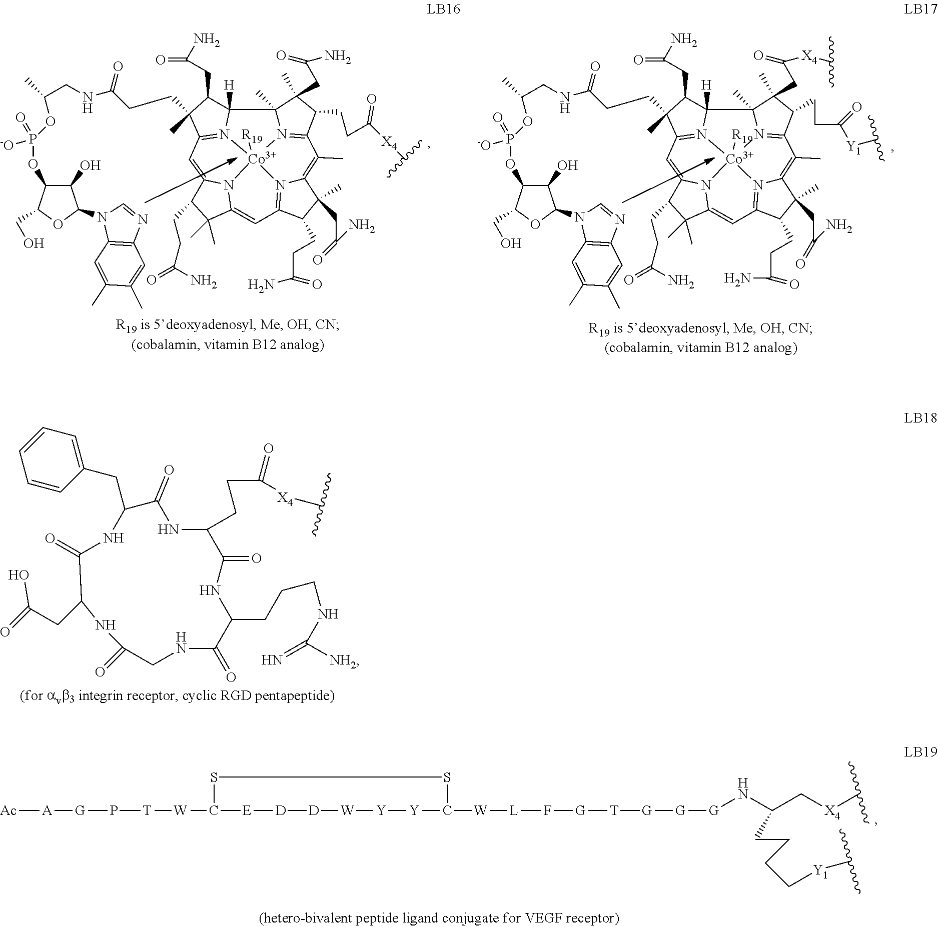 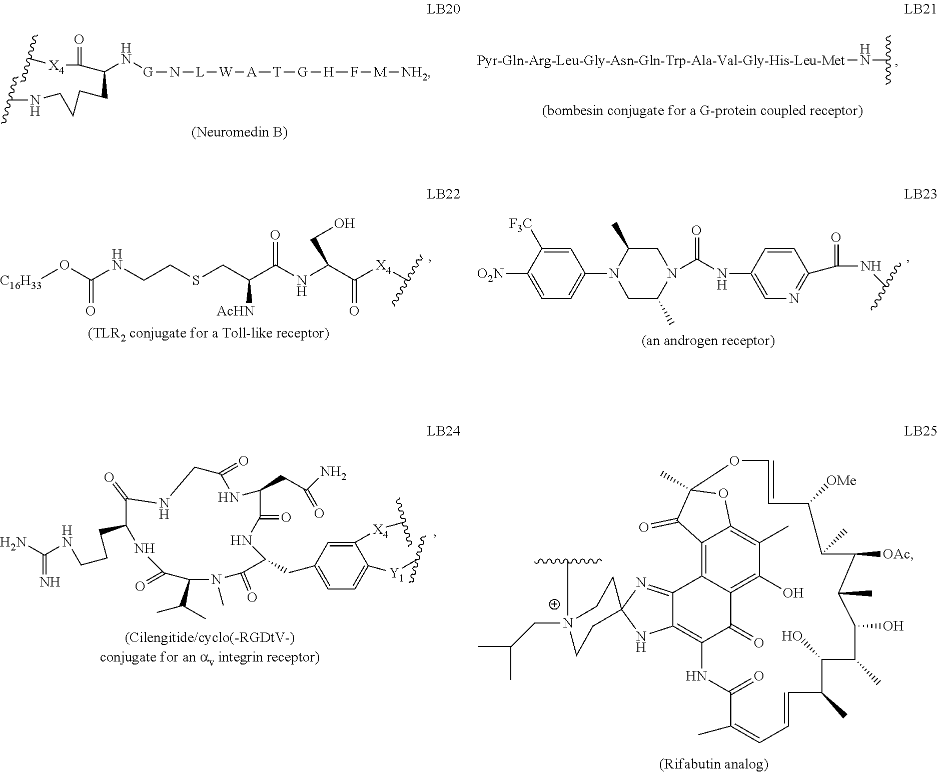 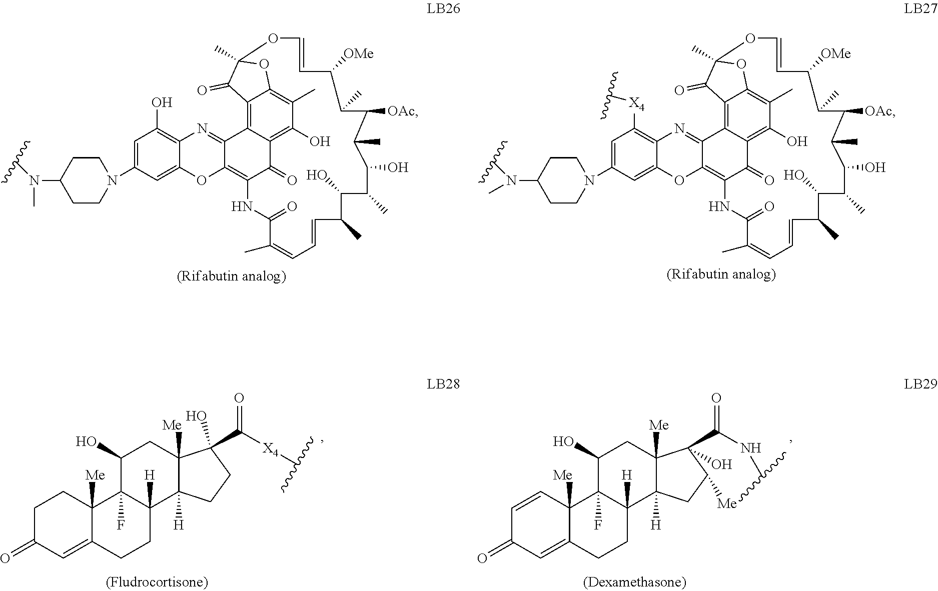 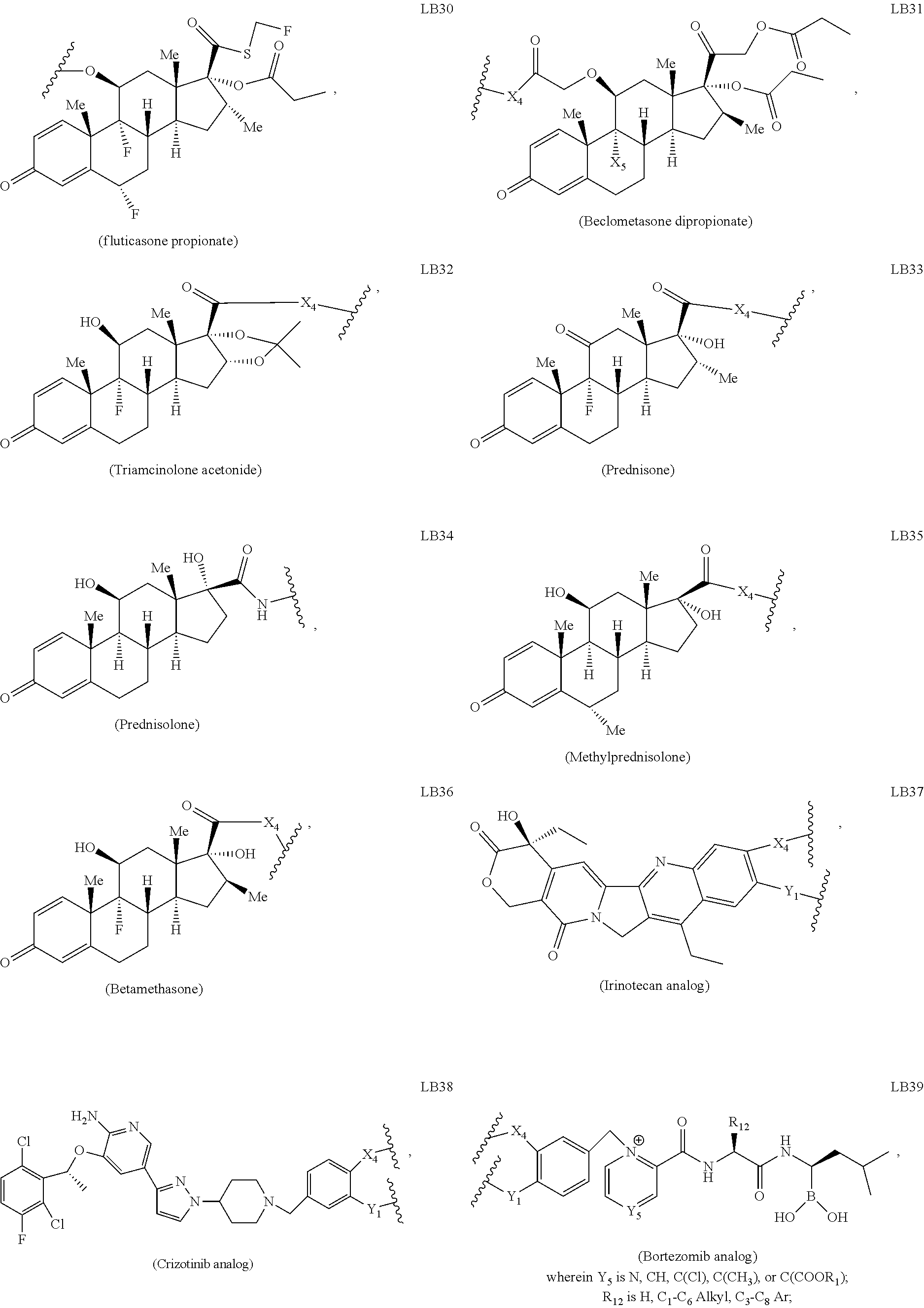 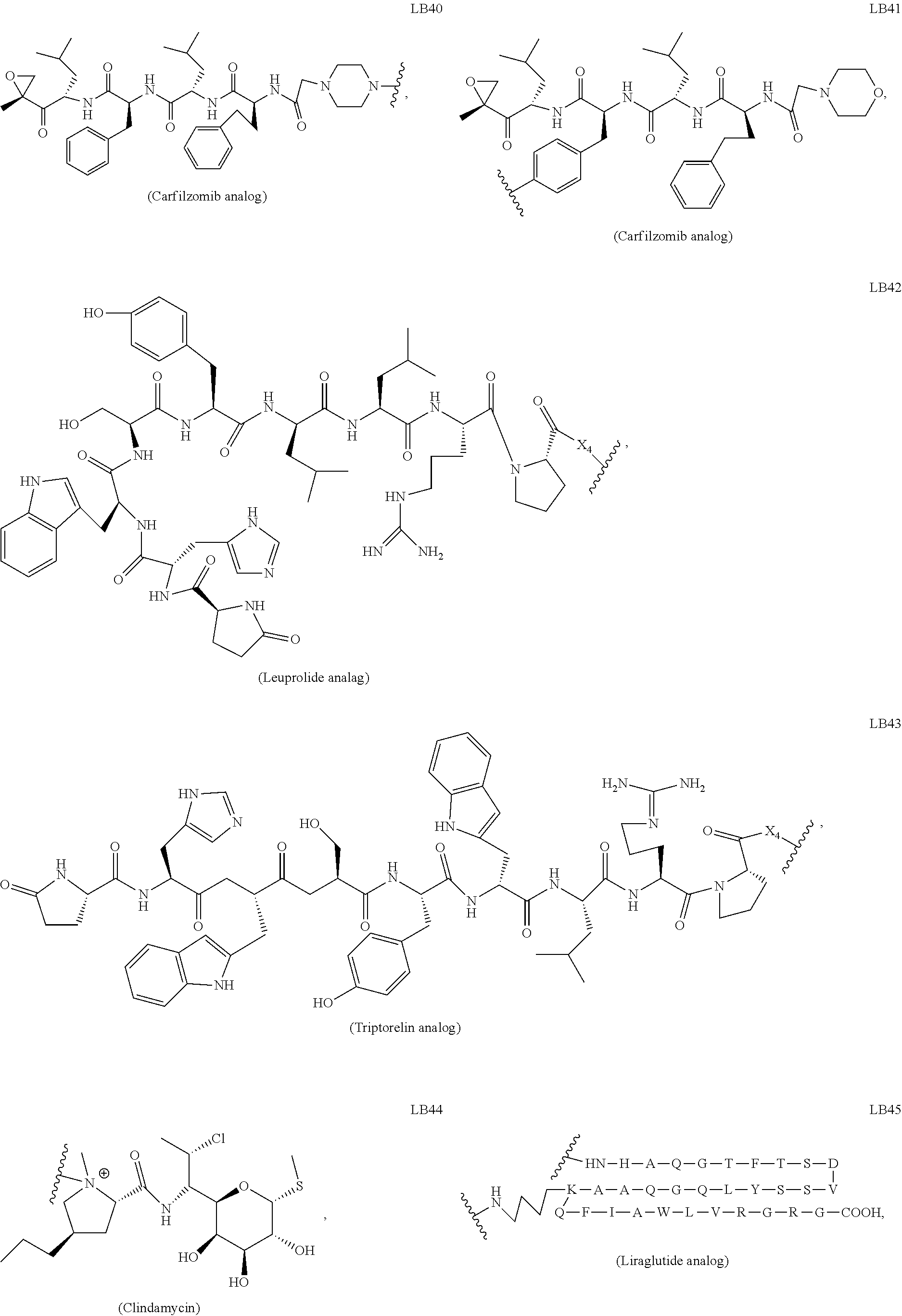 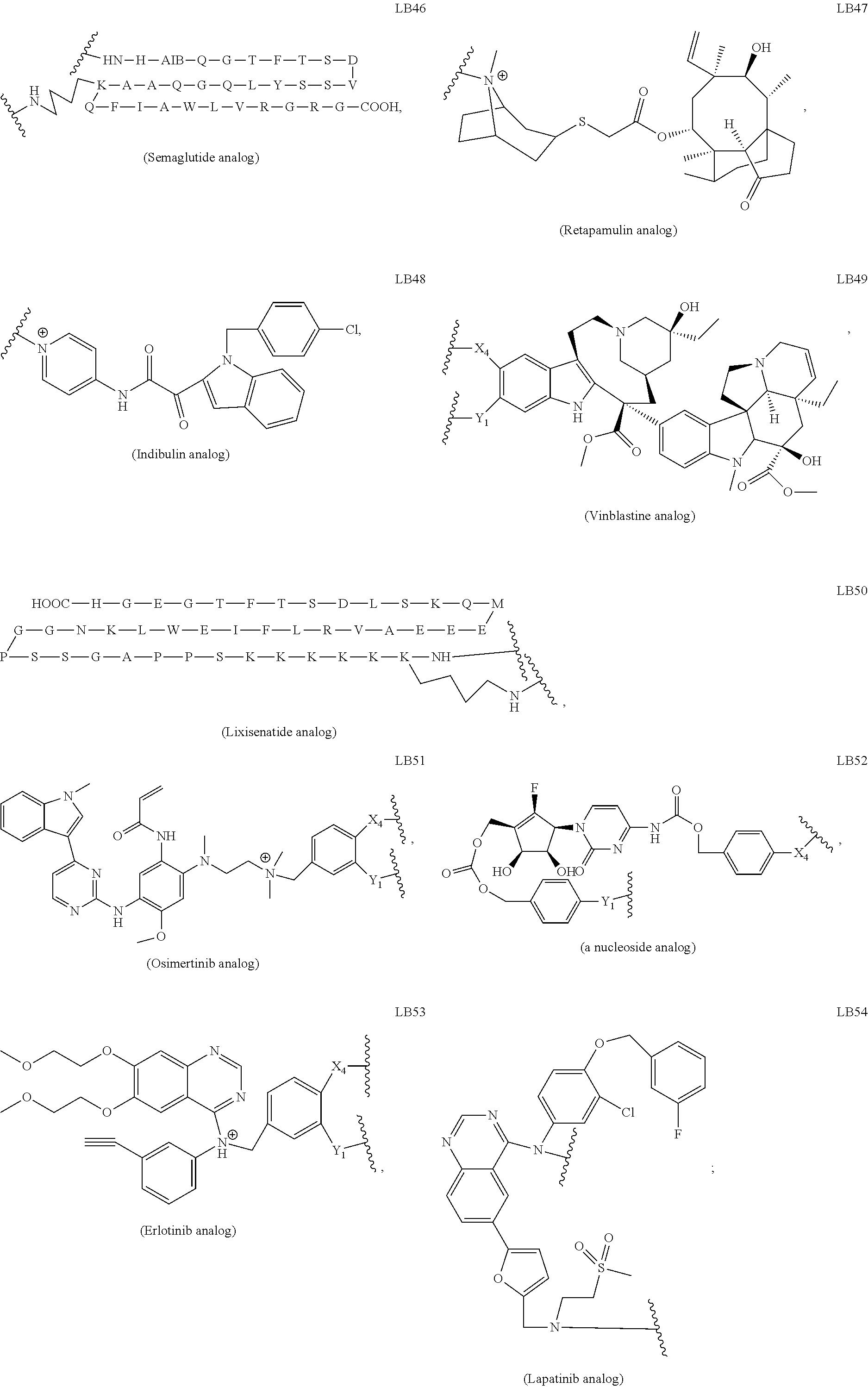
or an isotope of a chemical element, or a pharmaceutically acceptable salt, hydrates, or hydrated salt; or a polymorphic crystalline structure; or an optical isomer, racemate, diastereomer or enantiomer thereof, wherein [symbol] is the link site linked to L₁ or L₂; wherein X₄,and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁;
(w). the one, two or more DNA, RNA, mRNA, small interfering RNA (siRNA), microRNA (miRNA), and PIWI interacting RNAs (piRNA) can be as a chemotherapeutic/function compound conjugated to BCMA antibody having the following formula: 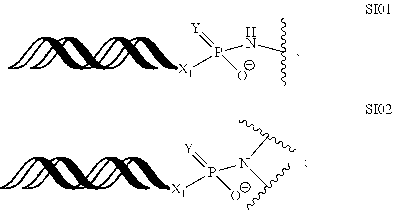
wherein “ [symbol] ” is the site to link the side chain linker; [symbol] is single or double strands of DNA, RNA, mRNA, siRNA, miRNA, or piRNA; X₁, and Y are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁.

43. The antibody-drug conjugate of claim 40, wherein the linker L₁ and L₂ are independently selected from the group consisting of:
O, NH, S, NHNH, N(R₃), N(R₃)N(R_{3′}), polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃][(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof, C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; Wherein R₃ and R₃, are independently H; C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of ester, ether, or amide; or 1˜8 natural or unnatural amino acids; or polyethyleneoxy unit of formula (OCH₂CH₂)ₚ or (OCH₂CH(CH₃))ₚ, wherein p is an integer from 0 to about 1000, or combination above thereof;
wherein L₁ or L₂ may contain a self-immolative or a non-self-immolative component, peptidyl units, a hydrazone bond, a disulfide, an ester, an oxime, an amide, or a thioether bond; the self-immolative unit includes the para-aminobenzylcarbamoyl (PAB) groups, including 2-aminoimidazol-5-methanol derivatives, heterocyclic PAB analogs, beta-glucuronide, and ortho or para-aminobenzylacetals having one of the following structures: 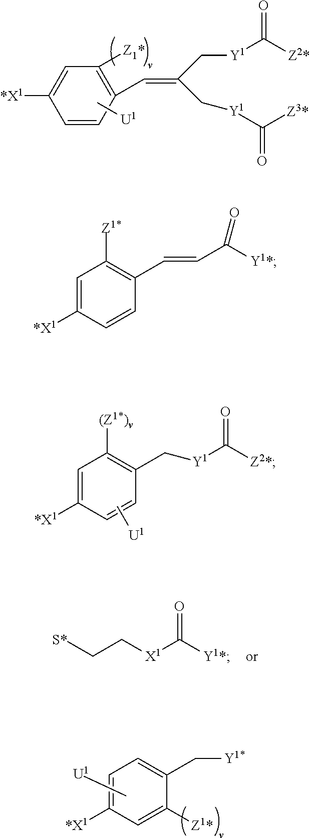
wherein the (*) atom is the point of attachment of additional spacer or releasable linker units, or the cytotoxic agent, and/or the binding molecule (CBA); X¹, Y¹, Z² and Z³ are independently NH, O, or S; Z¹ is independently H, NH, O or S; v is 0 or 1; U¹ is independently H, OH, C₁˜C₆ alkyl, (OCH₂CH₂)ₙF, Cl, Br, I, OR₅, SR₅, NR₅R₅′, N═NR₅, N═R₅,NR₅R₅′, NO₂, SOR₅R₅′, SO₂R₅, SO₃R₅, OSO₃R₅, PR₅R₅′, above; or pharmaceutical cation salts;
wherein the non-self-immolative linker component is one of the following structures: 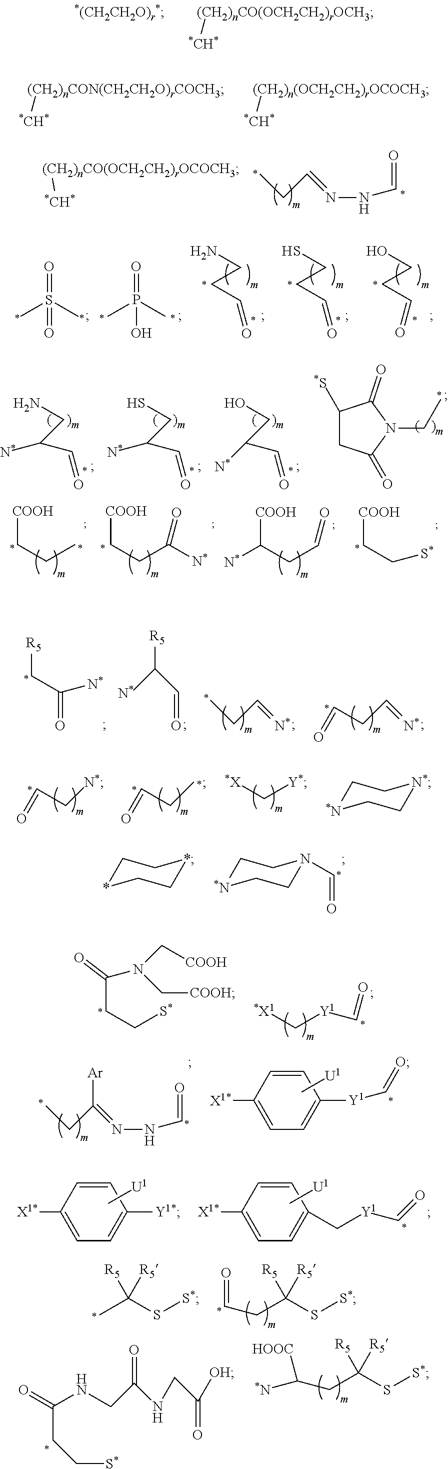 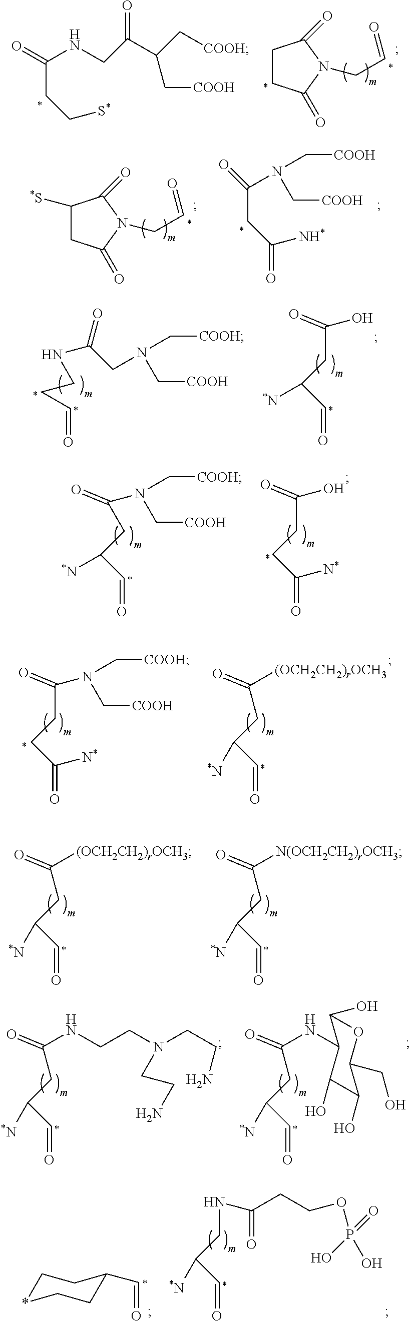 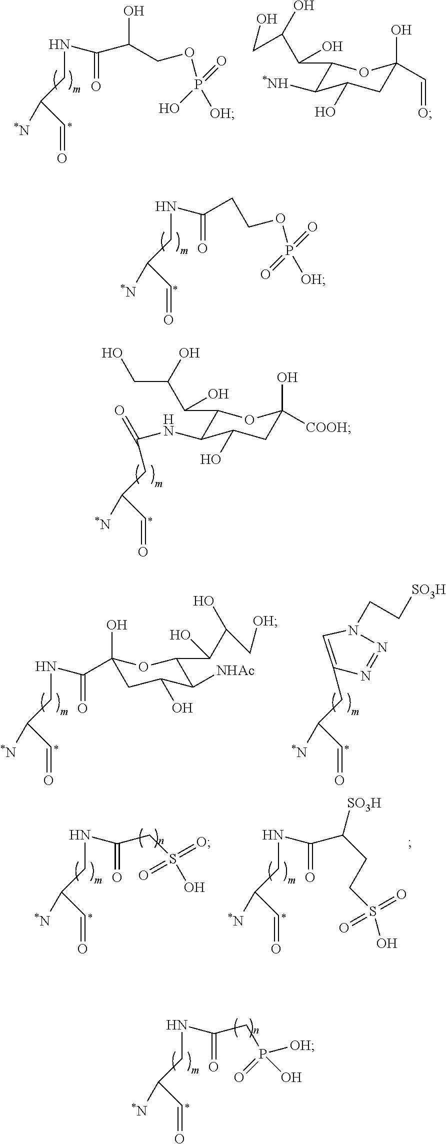
wherein the (*) atom is the point of attachment of additional spacer R₁ or releasable linkers, the cytotoxic agents, and/or the binding molecules; X¹, Y¹, U¹, R₁, R₅, R₅′ are defined as above; r is 0˜100; m and n are 0˜6 independently;
wherein L₁ or L₂ may be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites;
wherein L₁ or L₂ can be a releasable linker that includes at least one bond that can be a pH-labile, acid-labile, base-labile, oxidatively labile, metabolically labile, biochemically labile, or enzyme-labile bond; the component of releasable linkers (L₁ or L₂) include: —(CR₅R₆)ₘ(Aa)ᵣ-(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)ᵣ(CR₅R₆)ₘ(CR₇R₈)ₙ(OCH₂CH₂)ₜ—, —(CR₅R₆)ₘ(CR₇R₈)ₙ—(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)-(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ-(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ-(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ—(OCH₂CH₂)ᵣ—, —(CR₅R₆)ₘ-phenyl-CO(Aa)ₜ(CR₇R₈)ₙ-, —(CR₅R₆)ₘ-furyl-CO(Aa)ₜ(CR₇R₈)ₙ-, —(CR₅R₆)ₘ-oxazolyl-CO(Aa)ₜ(CR₇R₈)ₙ-, —(CR₅R₆)ₘ-thiazolyl-CO(Aa)ₜ-(CCR₇R₈)ₙ—, —(CR₅R₆)ₜ-thienyl-CO(CR₇R₈)ₙ-, —(CR₅R⁶)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R₆)ₘ-(Aa)ₜphenyl-, -(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(C R₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₘ(OCH₂CH₂)ₜ—, —K(CR₅R₆)ₘ—(CR₇R₈)ₙ(Aa)ᵣ(OCH₂CH₂)ₜ—, —K(Aa)ᵣ-(CR₅R₆)ₘ—(CR₇R₈)ₙ(OCH₂CH₂)ₜ—, —K(CR₅R₆)ₘ(CR₇R₈)ₙ—(OCH₂CH₂)ᵣ(Aa)ₜ-, —K(CR₅R₆)ₘ—(CR₇═CR₈)—(CR₉R₁₀)ₙ(Aa)ₜ(OCH₂CH₂)ᵣ—, —K(CR₅R₆)ₘ(NR₁₁CO)-(Aa)ₜ(CR₉R₁₀)ₙ(OCH₂CH₂)ᵣ—, —K(CR₅R₆)ₘ(Aa)ₜ-(NR₁₁CO)(CR₉R₁₀)ₙ(OCH₂CH₂)ᵣ—, —K(CR₅R₆)ₘ—(OCO)(Aa)ₜ(CR₉R₁₀)ₙ—(OCH₂CH₂)ᵣ—, —K(CR₅R₆)ₘ—(OCNR₇)(Aa)ₜ(CR₉R₁₀)ₙ(OCH₂CH₂)ᵣ—, —K(CR₅R₆)ₘ—(CO)(Aa)ₜ-(CR₉R₁₀)ₙ(OCH₂CH₂)ᵣ—, —K(CR₅R₆)ₘ—(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ—(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ-, —K(CR₅R₆)ₜimidazolyl-CO—(CR₇R₈)ₙ—, —K(CR₅R₆)ₜ-morpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ-N-methylpiperazinCO(Aa)ₜ(CR₇R₈)ₙ—, —K(CR₅R₆)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl-(Aa)ₜ-, —K(CR₅R₆)ₘ-thienyl-(Aa)ₜ-, —K(CR₅R₆)ₘ-imidazolyl-(Aa)ₜ-, —K(CR₅R₆)ₘ-morpholino(Aa)-, —K(CR₅R₆)ₘ-piperazino-(Aa)ₜG, —K(CR₅R₆)ₘ—N-methyl-piperazino(Aa)ₜ; wherein m, Aa, m, n, R₃, R₄, and R₅ are described above; t and r are 0-100 independently; R₆, R₇, and R₈ are independently chosen from H; halide; C₁˜C₈ of alkyl, aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B or C₃-C₆ heteroaromatic group;
wherein the structures of the components of the linker L₁ and/or L₂ are: 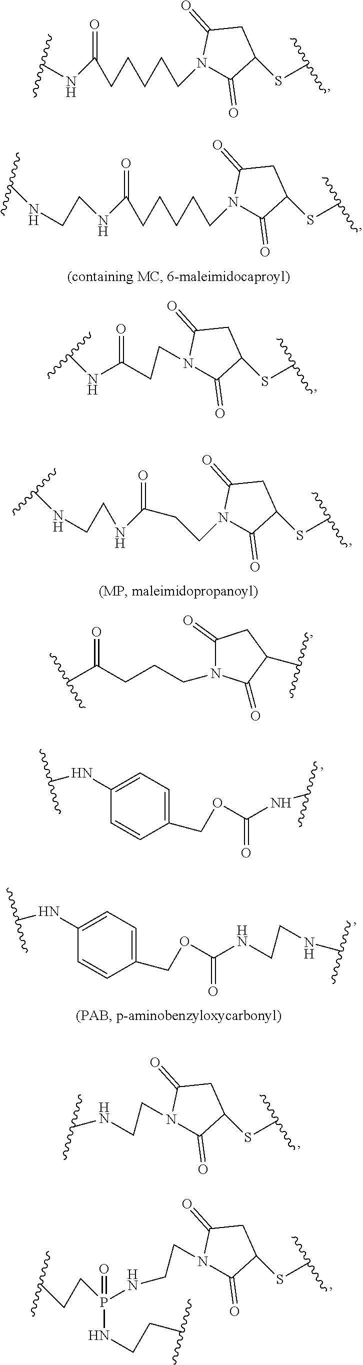 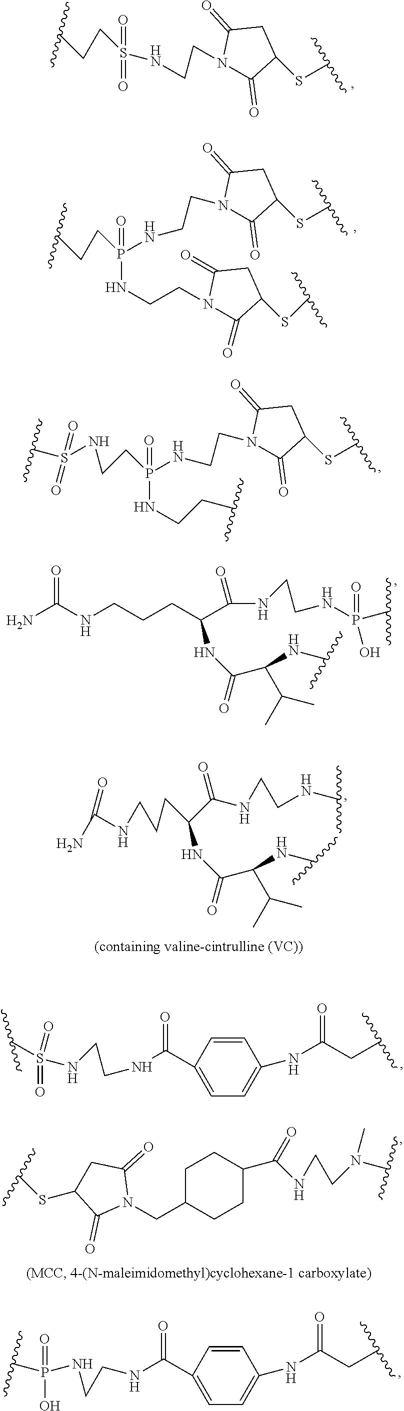 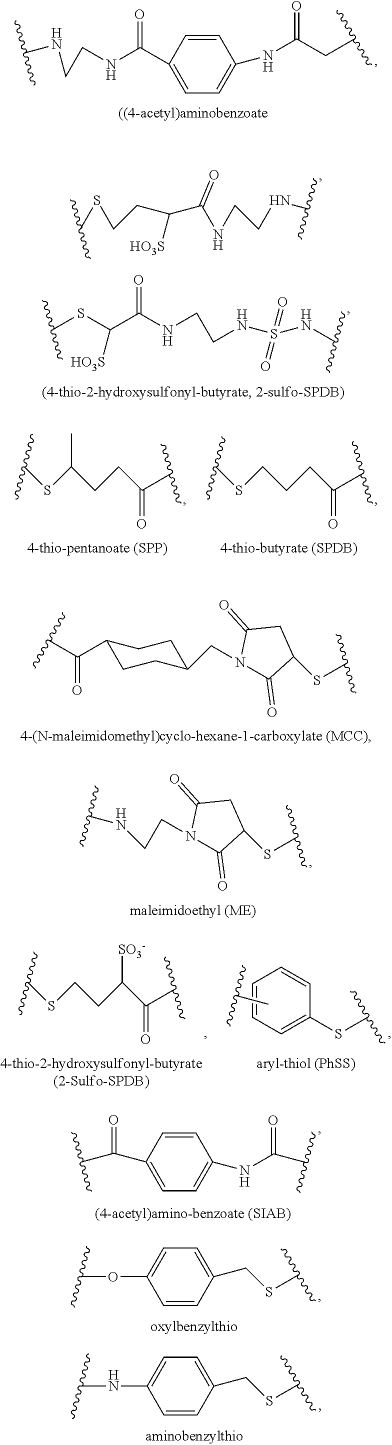 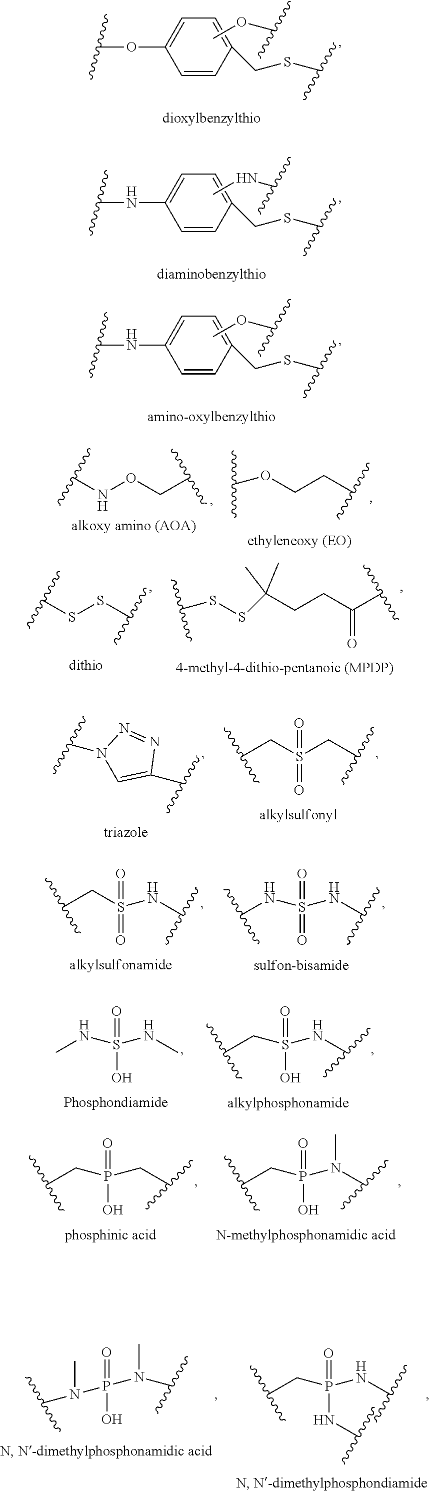 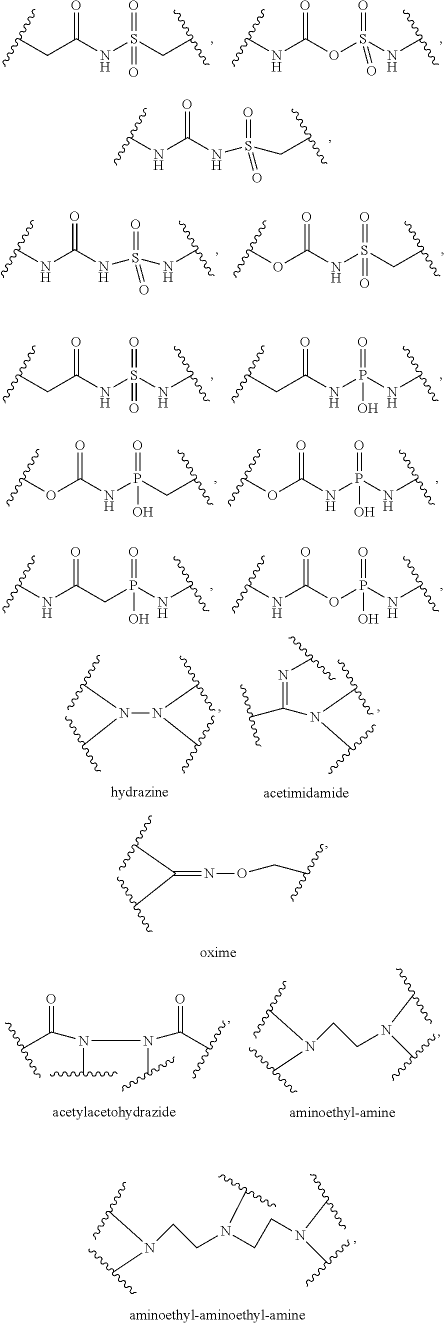 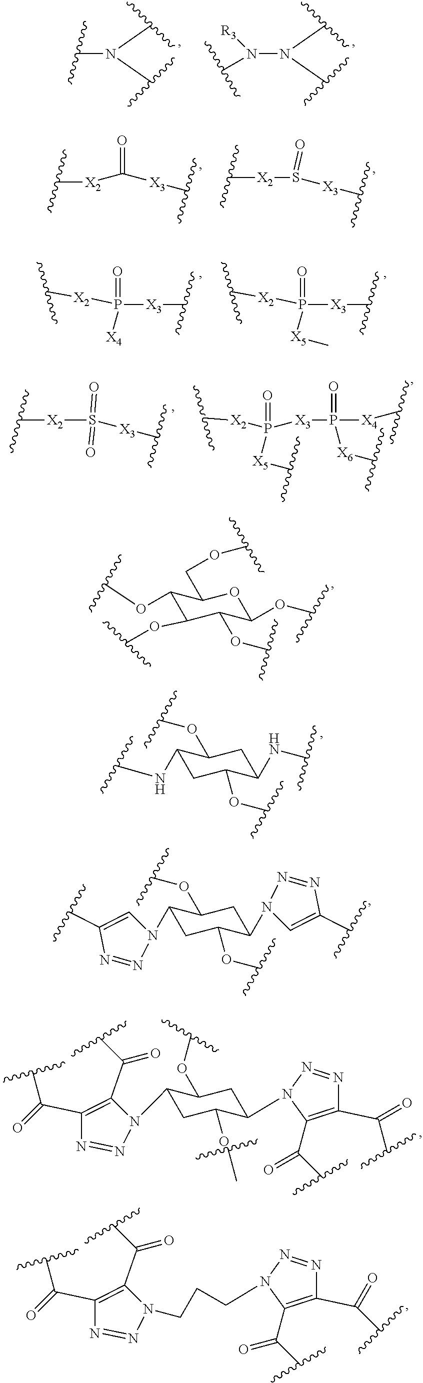 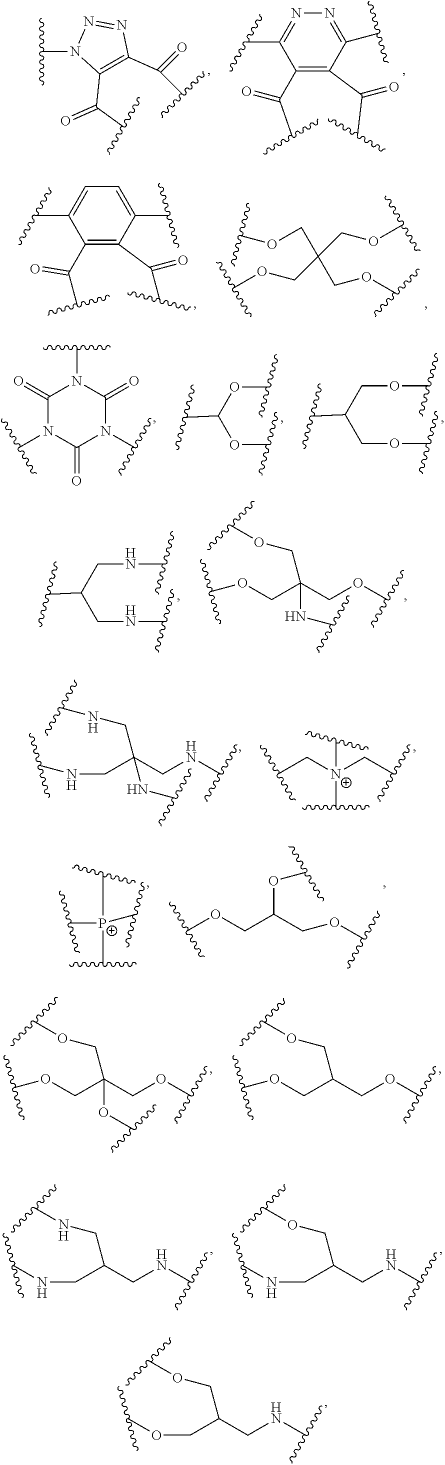 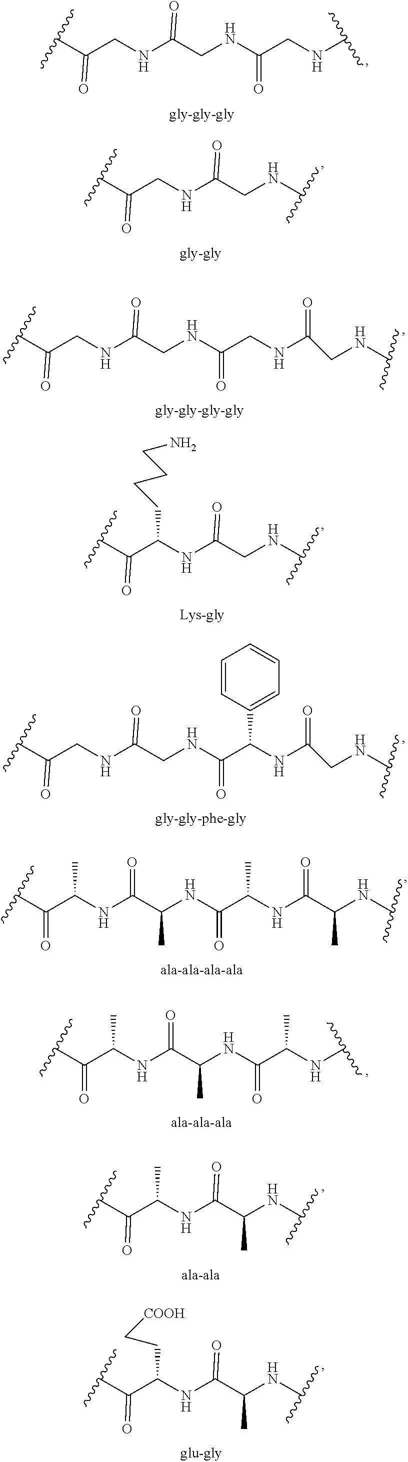 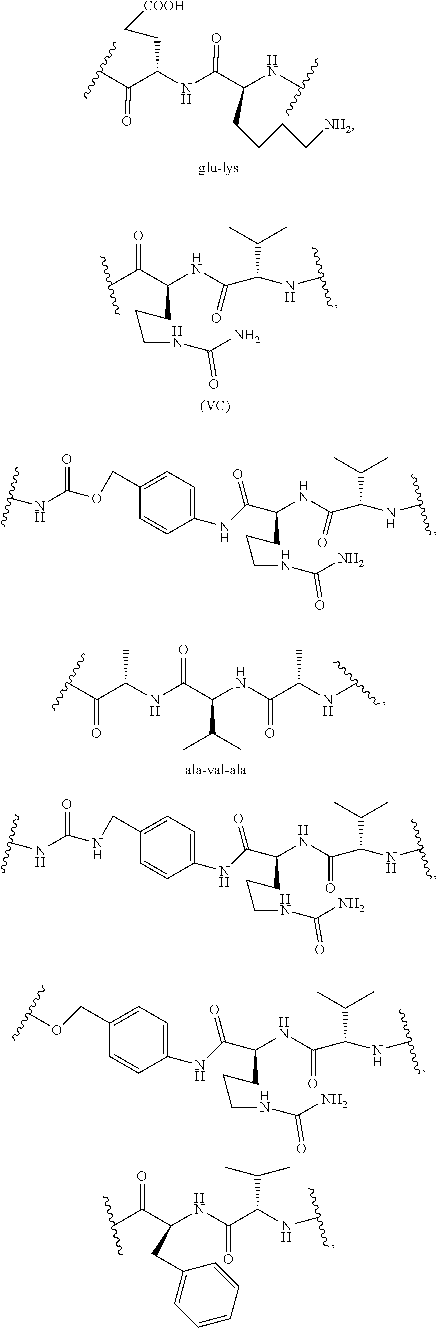 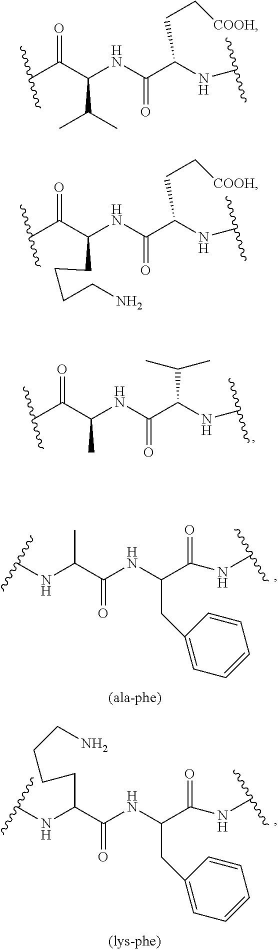 [SMILES: *N[C@@H](CCC(=O)O)C(=O)N[C@H](C(*)=O)C(C)C ; *N[C@@H](CCC(=O)O)C(=O)N[C@@H](CCCCN)C(*)=O ; *N[C@H](C(=O)N[C@@H](C)C(*)=O)C(C)C ; *NC(=O)C(Cc1ccccc1)NC(=O)C(C)N* ; *NC(=O)C(Cc1ccccc1)NC(=O)C(CCCCN)N*]
or a combination above thereof; wherein [symbol] is the site of linkage; X₂, X₃, X₄, X₅, or X₆, are independently selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of ester, ether, or amide; or polyethyleneoxy unit of formula (OCH₂CH₂)ₚ or (OCH₂CH(CH₃))ₚ, wherein p is an integer from 0 to about 100, or a combination thereof.

44. The antibody-drug conjugate of claim 40, wherein the linker L₁ and L₂ are independently selected from the group consisting of: 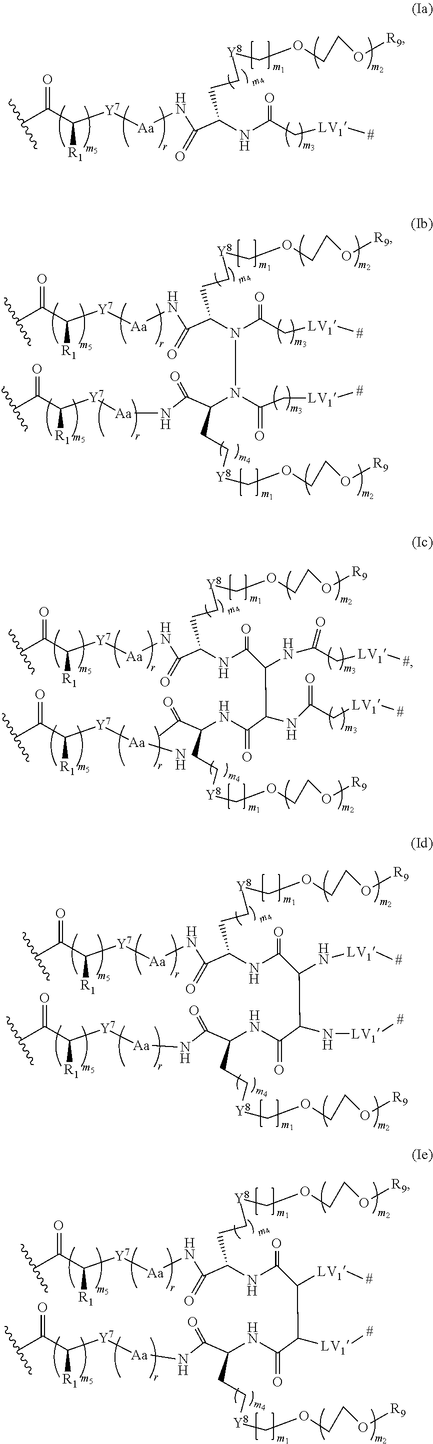
wherein “ [symbol] ” is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; Aa is L- or D-natural or unnatural amino acids;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0-12; when r is not 0, (Aa)ᵣ is the same or different amino acids or peptide units;
m₁=1-18; m₂=1-100; m₃=1-8; m₄=0-8; m₅=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y⁸ is NHC(═O), NHS(O₂), NH(SO), NHS(O₂)NH, NHP(O)(OH)NH or C(O)NH;
R₉ is H, (O═)CR₁, (O═)CNHR₁, R₁COOH, R₁(COCH₂NH)ₘ₂H, R₁(Aa)ᵣ or R₁(COCH₂NCH₃)ₘ₂H, wherein R₁ is defined above;
Lv₁′ is selected from: 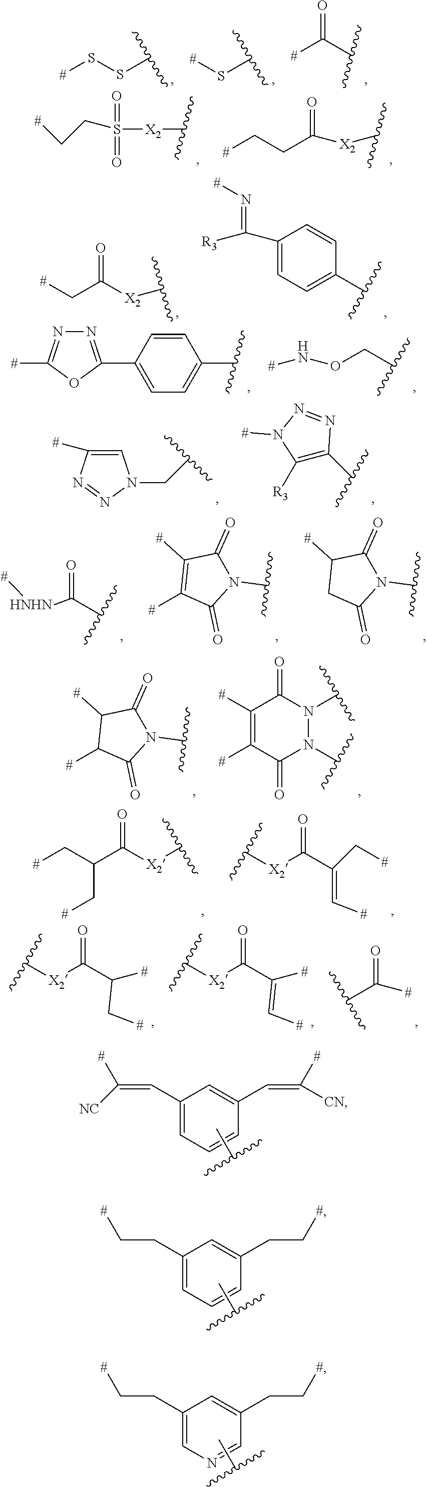 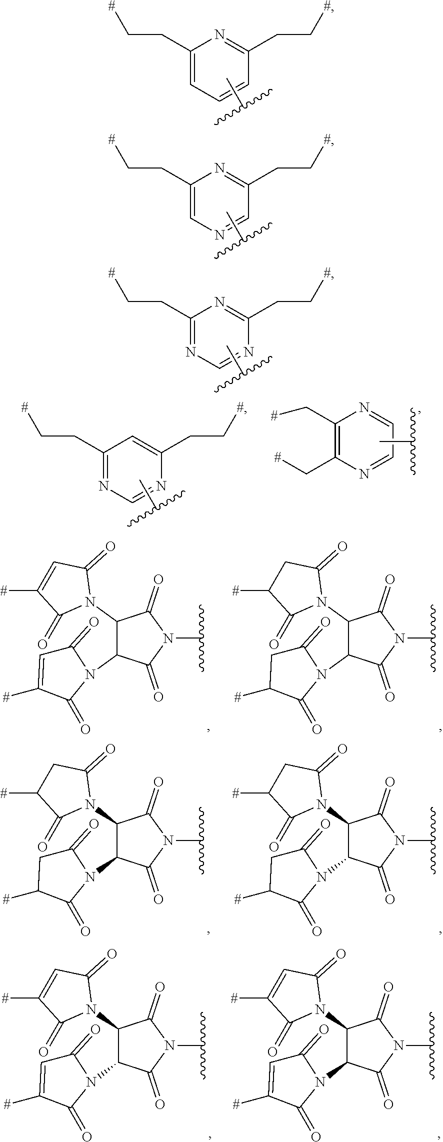 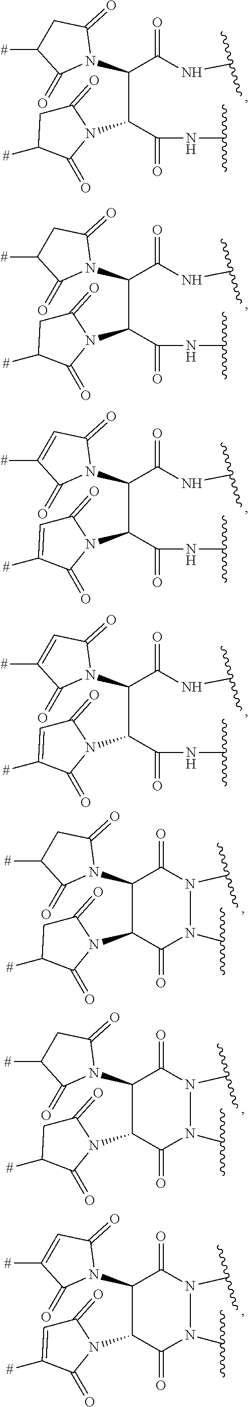 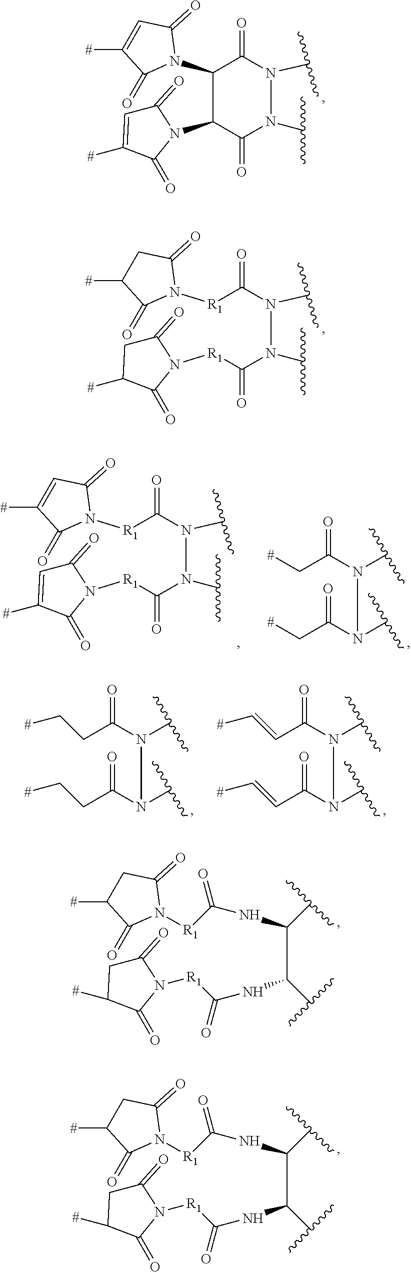 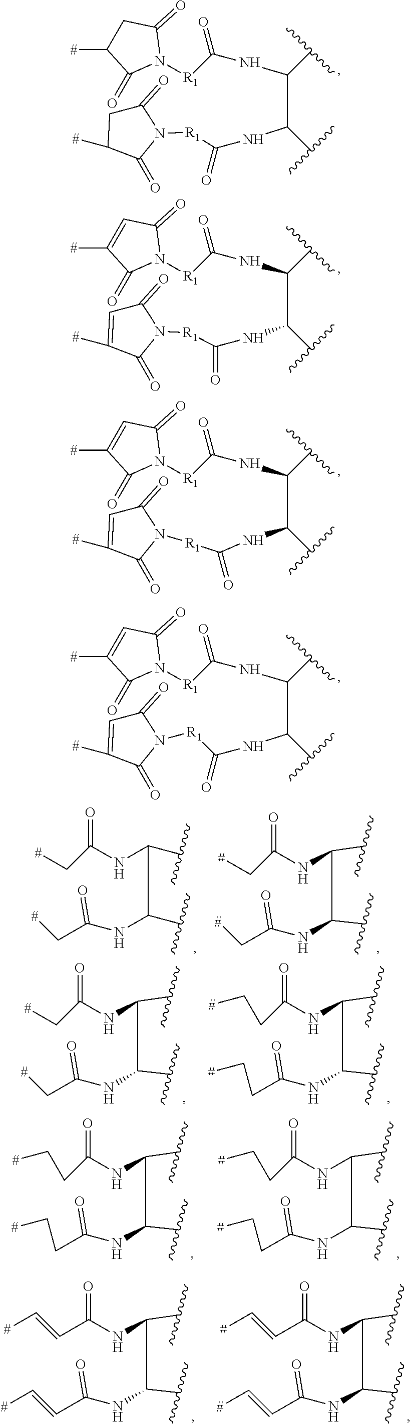 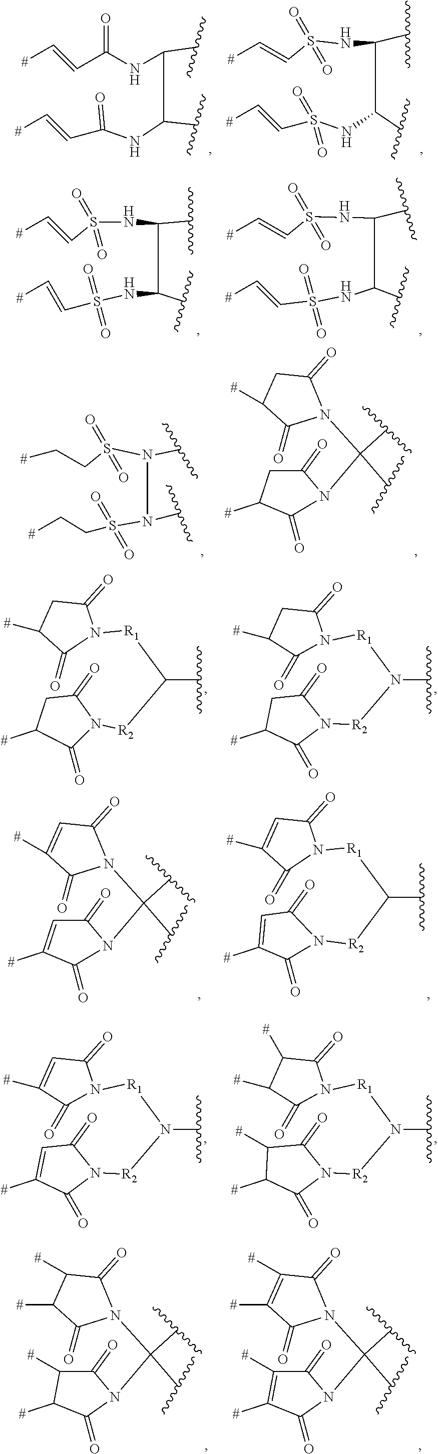 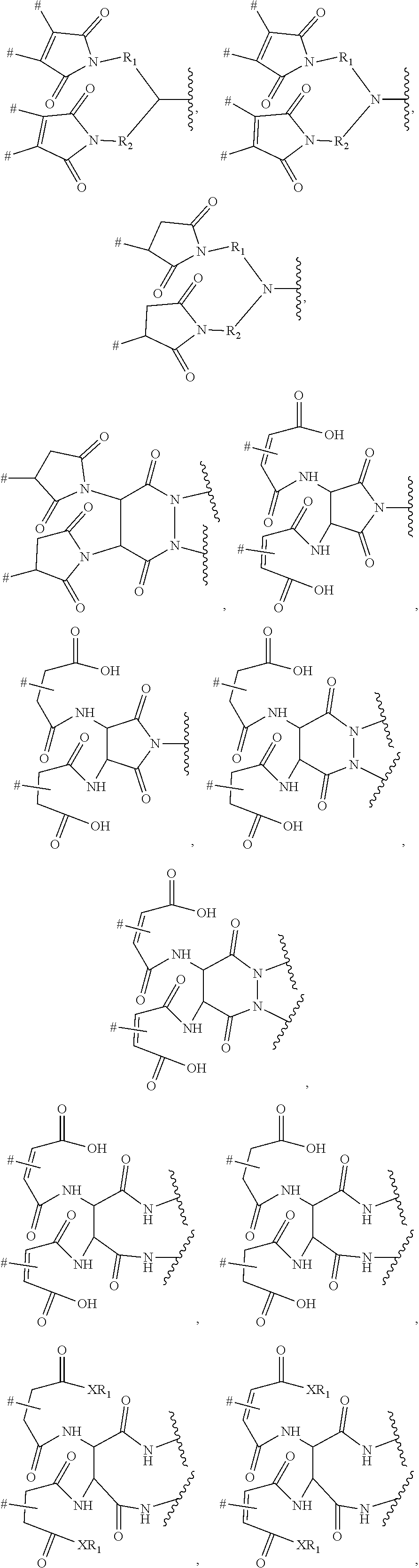 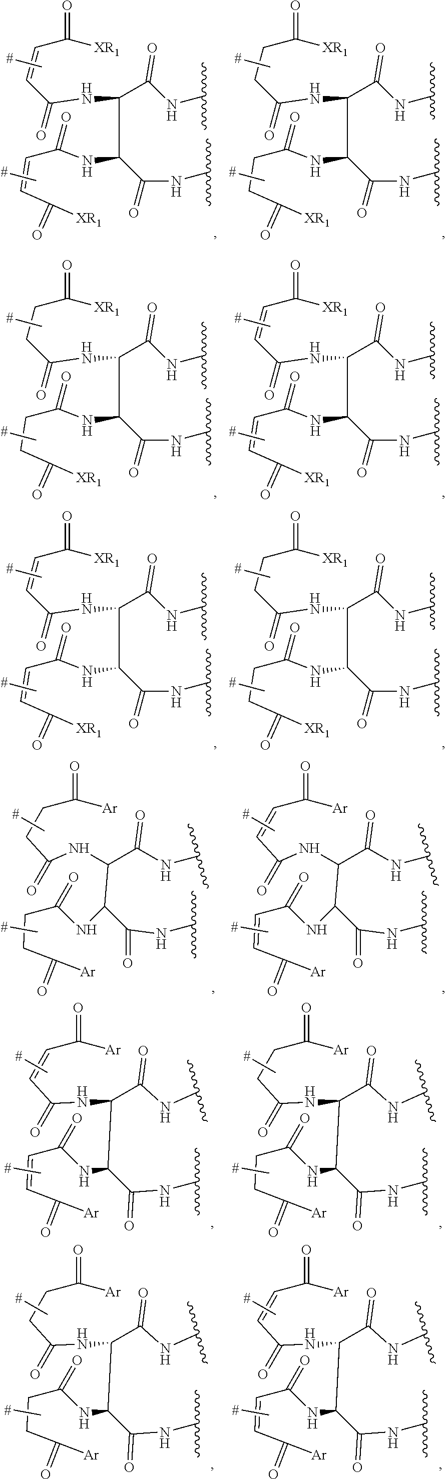 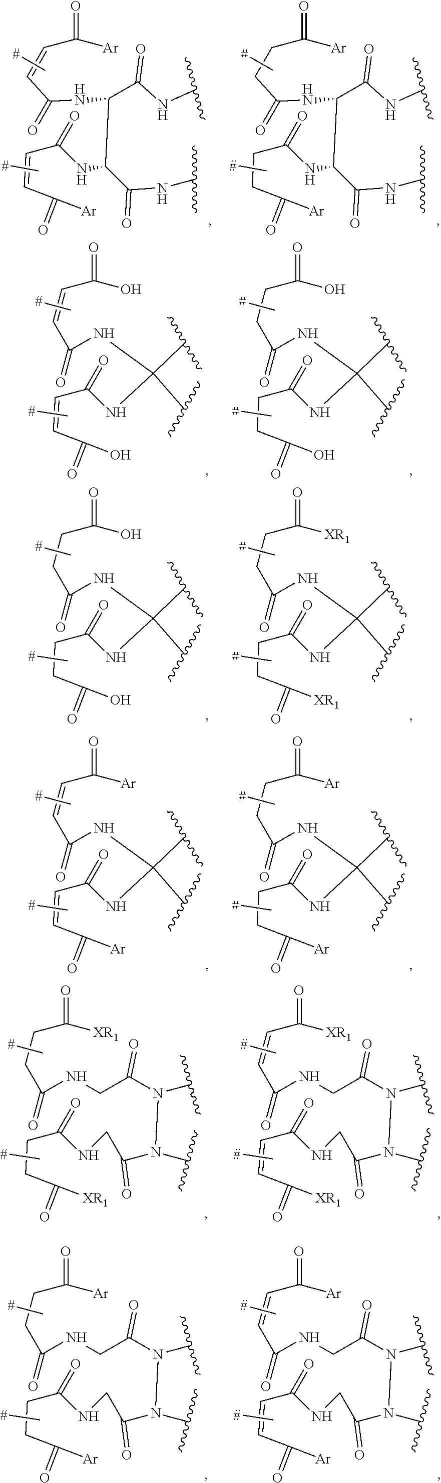 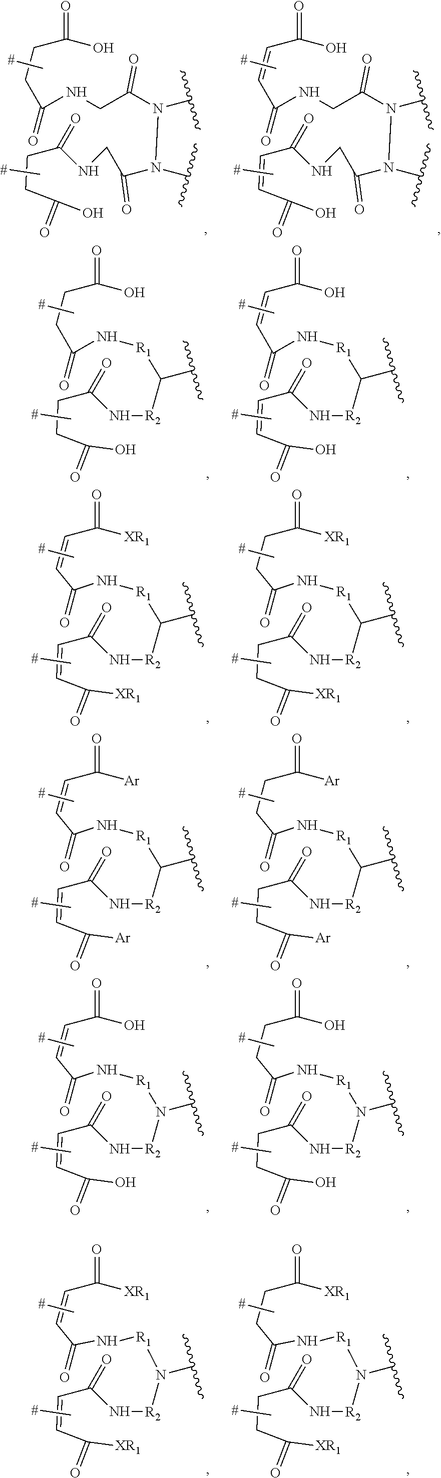 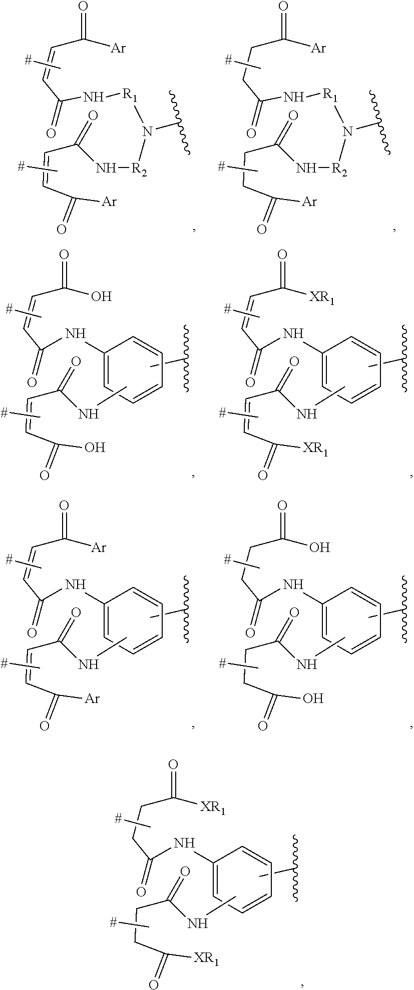 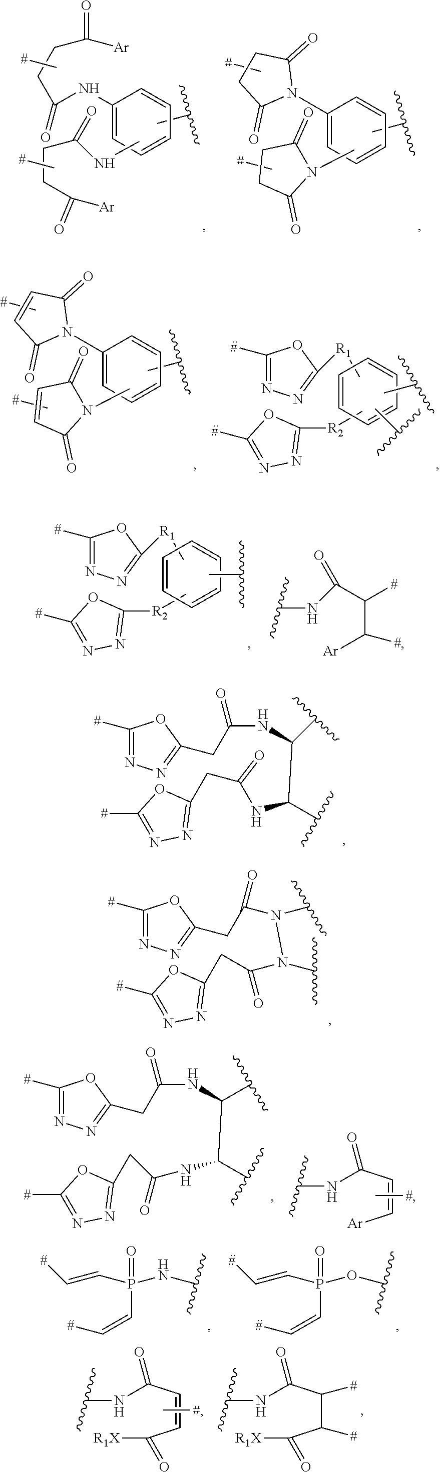
wherein “ [symbol] ” is a site that links a drug or a site of linker L₁ or L₂; “#” is a site that links a S (99 fa), O (phenol), NH (amino), CHO (aldehyde), C(═O) (ketone), C(O)(NH) (amide) and C(O)(OH) (carboxylate) 99 fan antibody; wherein R₁, X₁′ and X₂′ are described above; X is O, NH, S, CH₂; the connecting bond “ [symbol] ” in the middle of the two atoms means it can link either one of the two atoms, Ar is an aromatic group.

45. The antibody-drug conjugate of claim 40, wherein the linker L₁ and L₂ are independently the following formula (If) or (Ig): 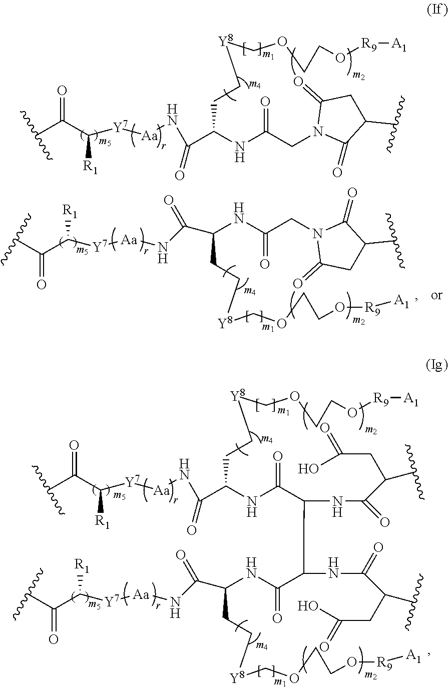
wherein Aa is L- or D-natural or unnatural amino acids;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0-12; when r is not 0, (Aa)ᵣ is the same or different sequences of amino acids or peptide units;
m₁=1-18; m₂=1-100; m₃=1-8; m₄=0-8; m₅=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y⁸ is NHC(═O), NHS(O₂), NH(SO), NHS(O₂)NH, NHP(O)(OH)NH or C(O)NH;
R₉ is H, (O═)CR₁, (O═)CNHR₁, R₁COOH, R₁(COCH₂NH)ₘ₂H, R₁(Aa)ᵣ or R₁(COCH₂NCH₃)ₘ₂H, wherein R₁ is defined above.

46. A method for preparing the antibody-drug conjugate of claim 40 comprising reacting a drug/linker complex having a formula (IV), (V) or (VI) below to amino acids of an antibody to form the conjugate of formula (I), (II), or (III): 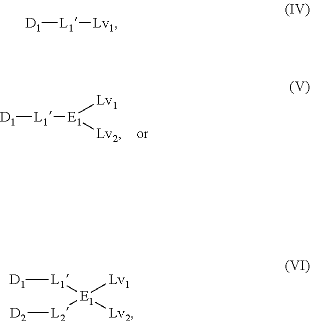
wherein: Lv₁ and Lv₂ are a reactive group, and are independently selected from: 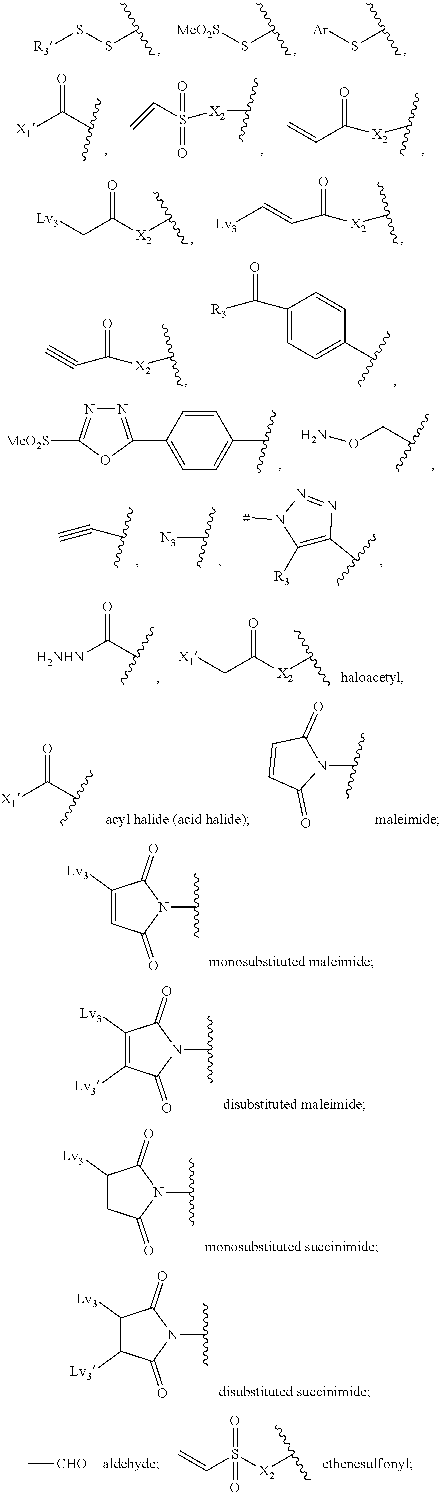 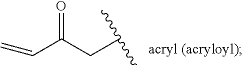 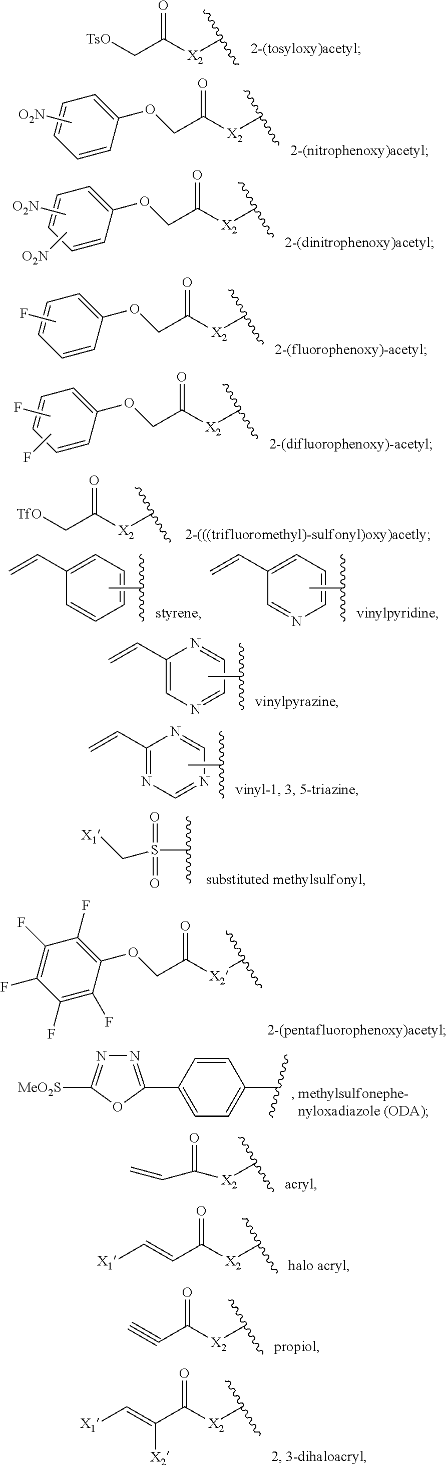 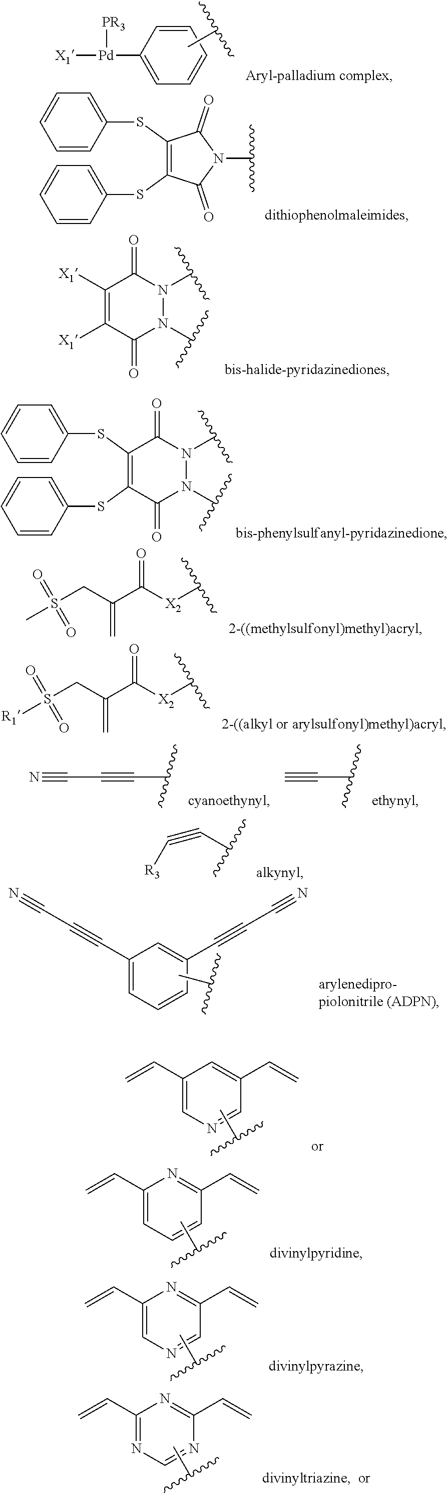 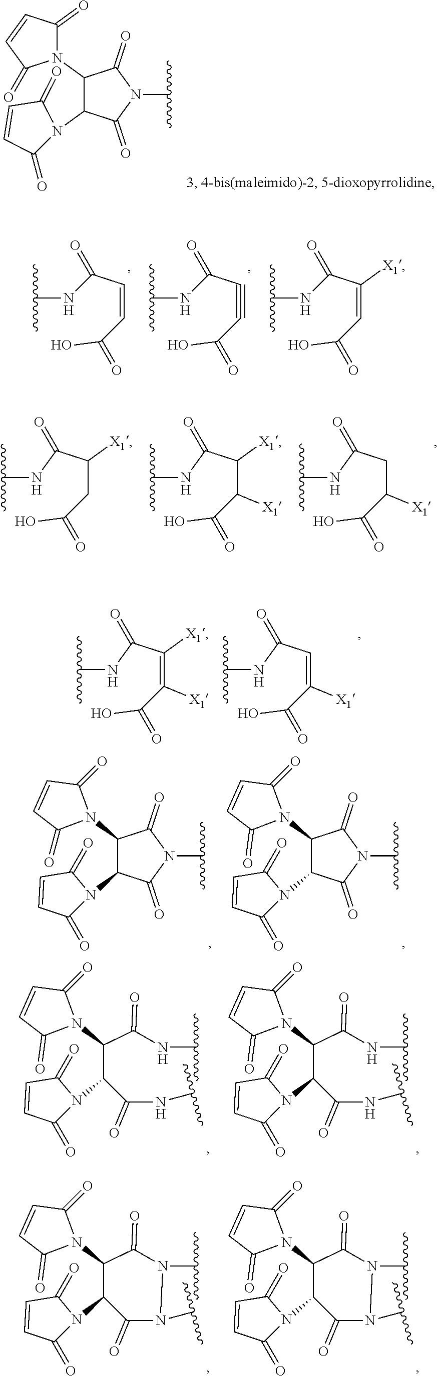 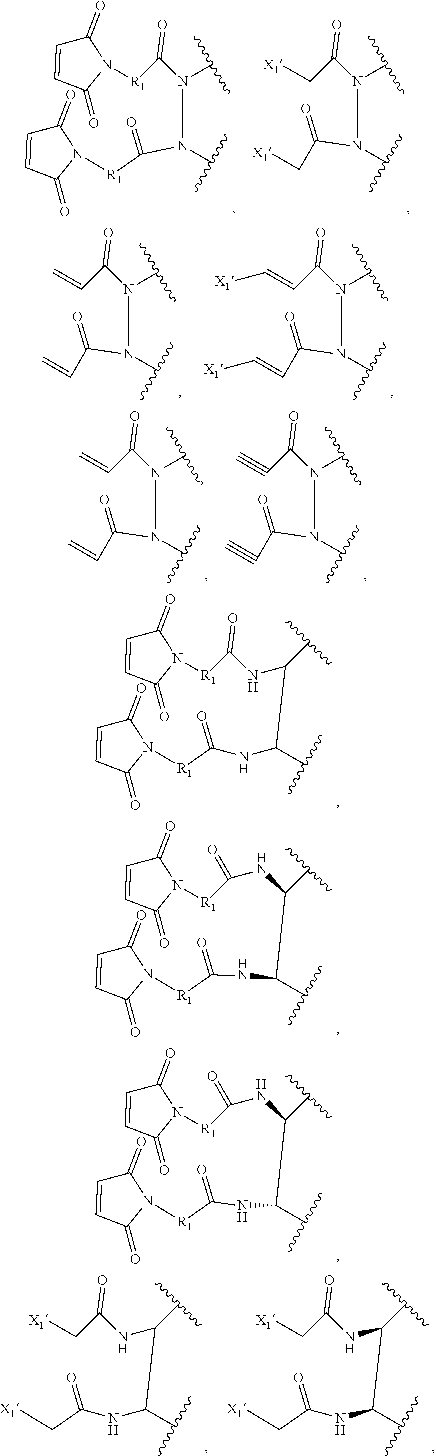 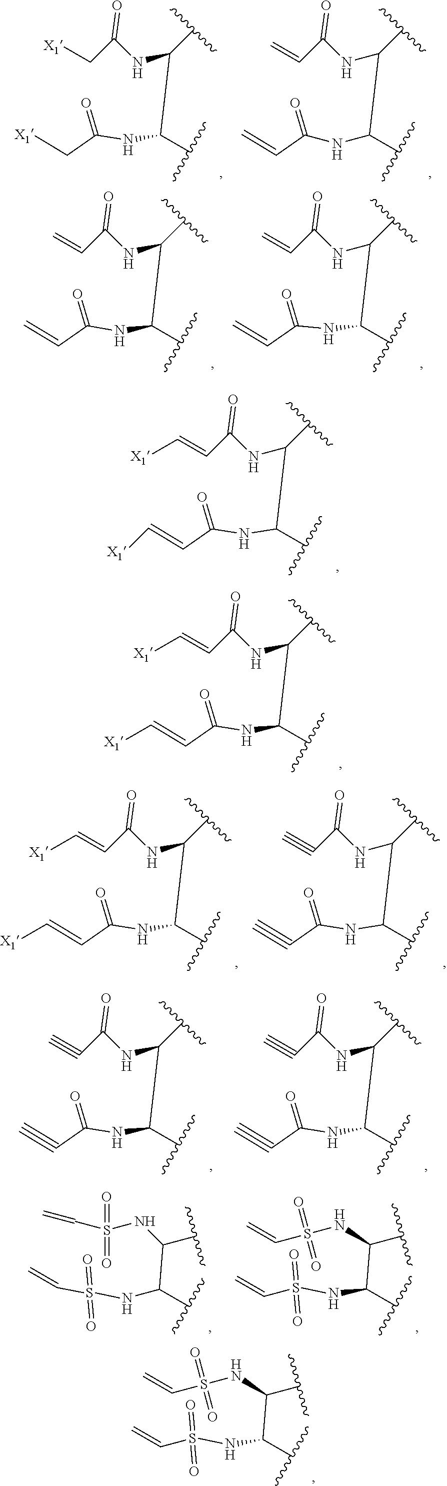 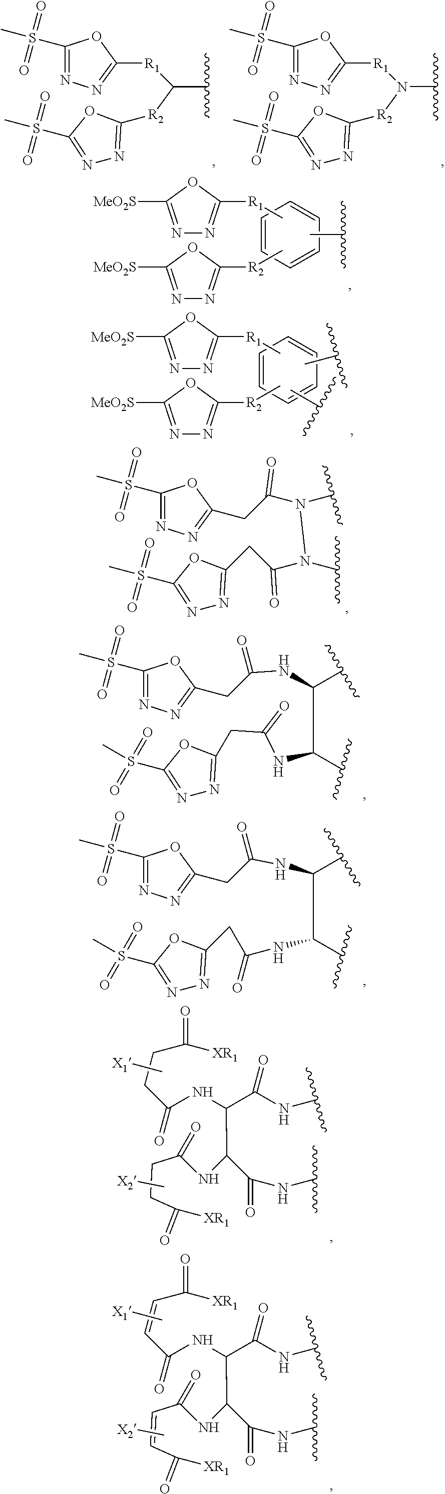 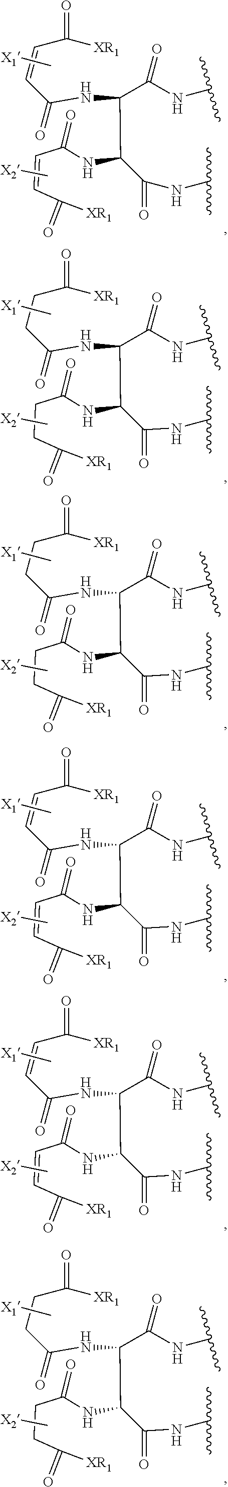 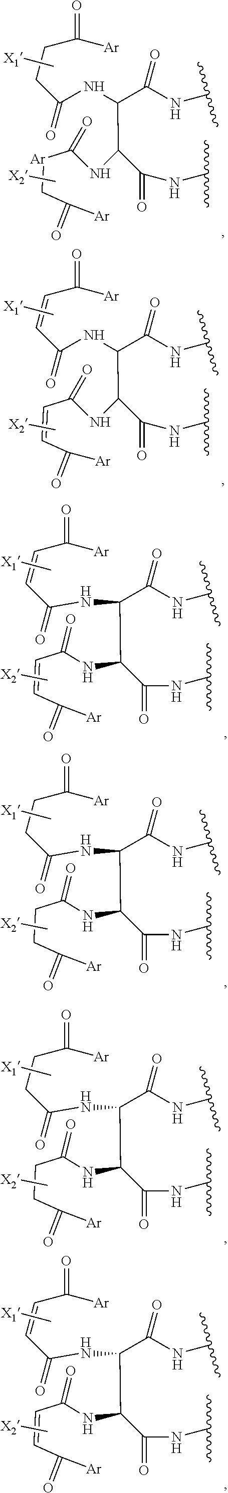 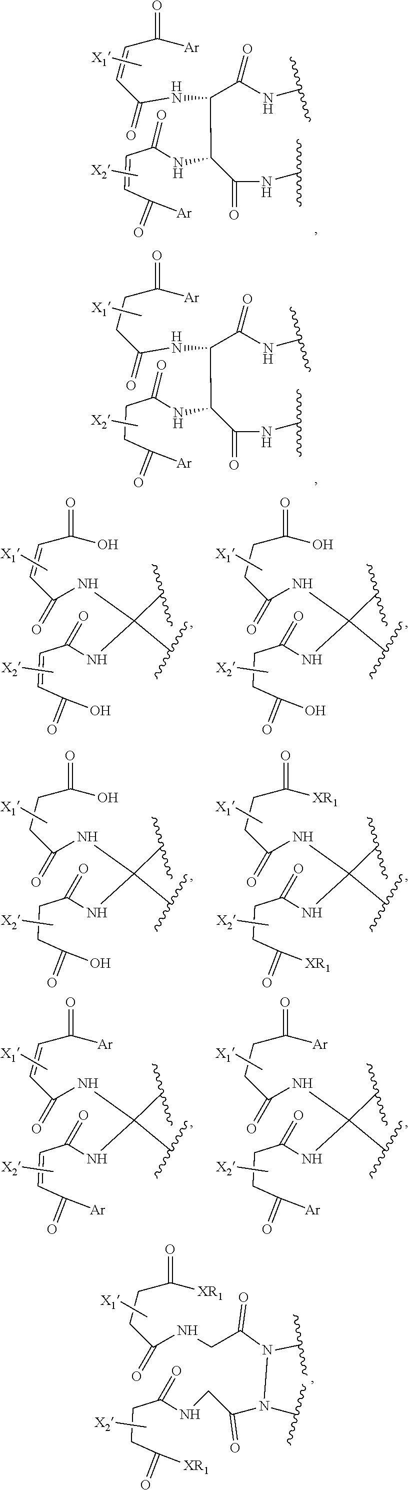 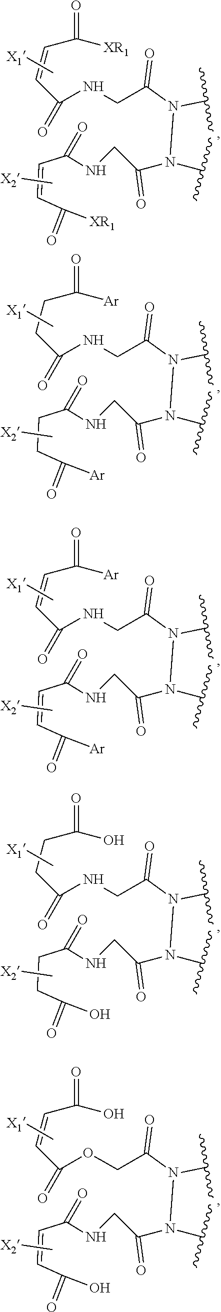 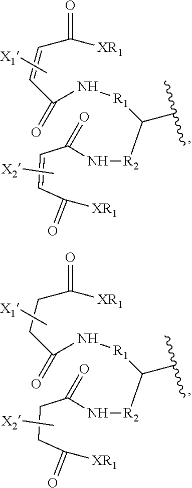 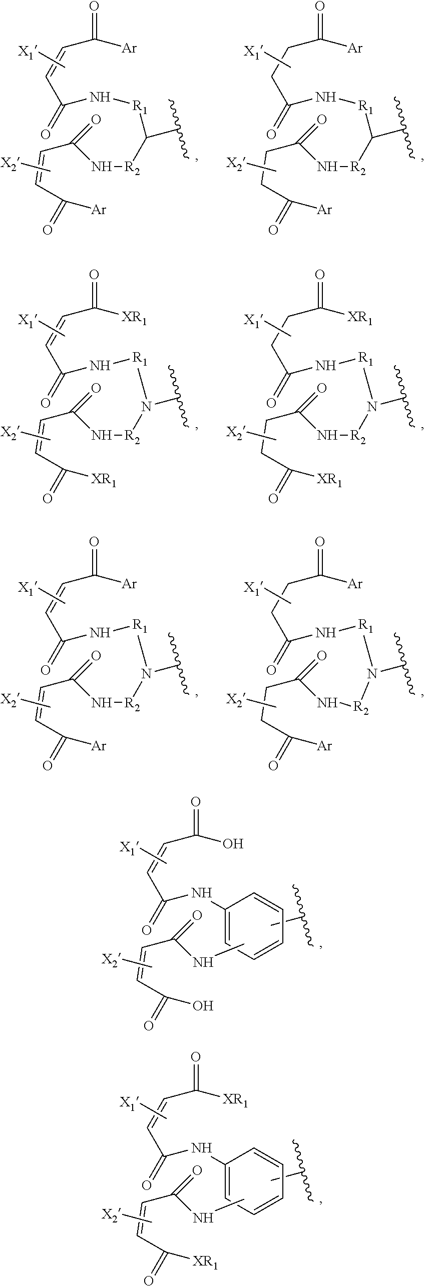 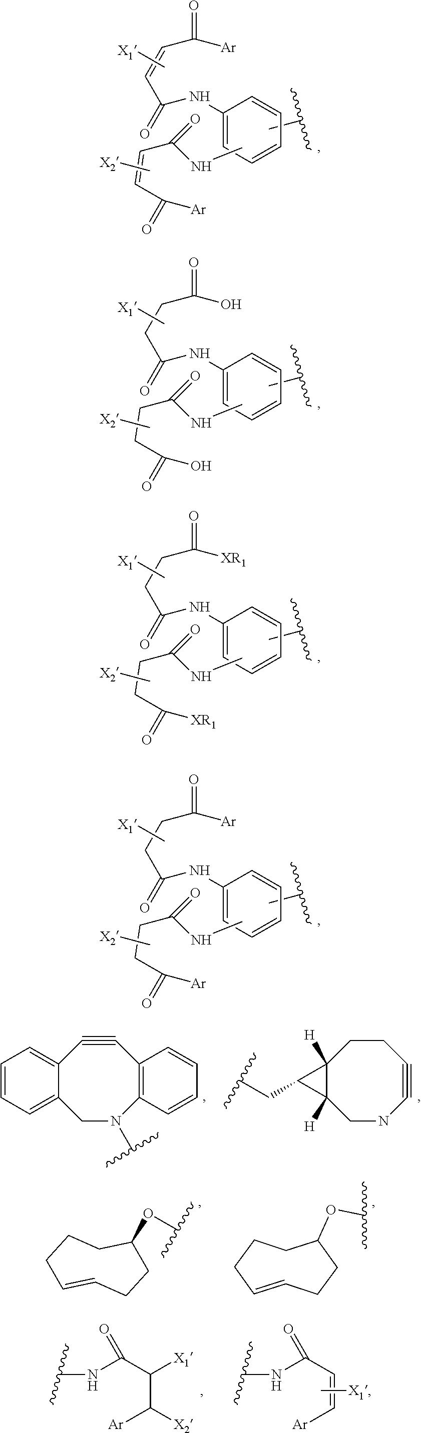 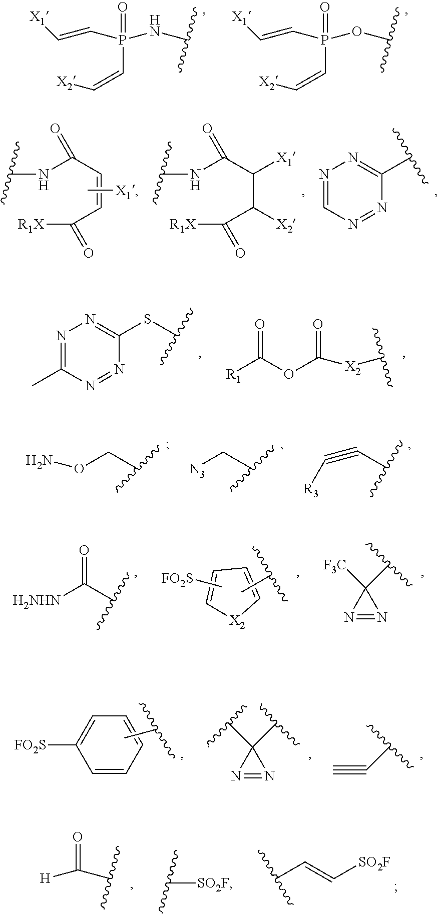
wherein X₁′ and X₂′ are independently F, Cl, Br, I, OTf, Oms, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;
L₁′ and L₂′ are, the same or different, independently selected from O, NH, S, S—S, NHNH, N(R³), N(R₃)N(R_{3′}), C₁-C₈ of alkyl; C₂-C₈ of heteroalkyl, alkylcycloalkyl, or heterocycloalkyl; C₃-C₈ of aryl, Ar-alkyl, heterocyclic, carbocyclic, cycloalkyl, heteroalkylcycloalkyl, alkylcarbonyl, or heteroaryl; C₂-C₈ (2-8 carbon atoms) of ester, ether, or amide; 1˜8 natural or unnatural amino acids described in the definition; polyethyleneoxy unit of formula (OCH₂CH₂)ₚ, (OCH₂CH(CH₃))ₚ, (OCH₂CH₂)ₚOR₃, (OCH₂CH(CH₃))ₚOR₃, NH(CH₂CH₂O)ₚR₃, NH(CH₂CH(CH₃)O)ₚR₃, N[(CH₂CH₂—O)ₚR³][(CH₂CH₂O)ₚR₃′], (OCH₂CH₂)ₚCOOR₃, or CH₂CH₂(OCH₂CH₂)ₚCOOR₃, wherein p and p′ are independently an integer selected from 0 to about 1000, or combination thereof, wherein R₃ and R₃′ are independently H; C₁-C₈ of alkyl; C₂-C₈ of heteroalkyl, alkylcycloalkyl, or heterocycloalkyl; C₃-C₈ of aryl, Ar-alkyl, heterocyclic, cycloalkyl, heteroalkylcycloalkyl, alkylcarbonyl, or heteroaryl;
L₁′ and/or L₂′ may contain a self-immolative or a non-self-immolative component, peptidyl units, a hydrazone bond, a disulfide, an ester, an oxime, an amide, or a thioether bond;
wherein the components of 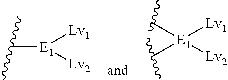
in the formula (V) and formula (VI) are independently selected from: 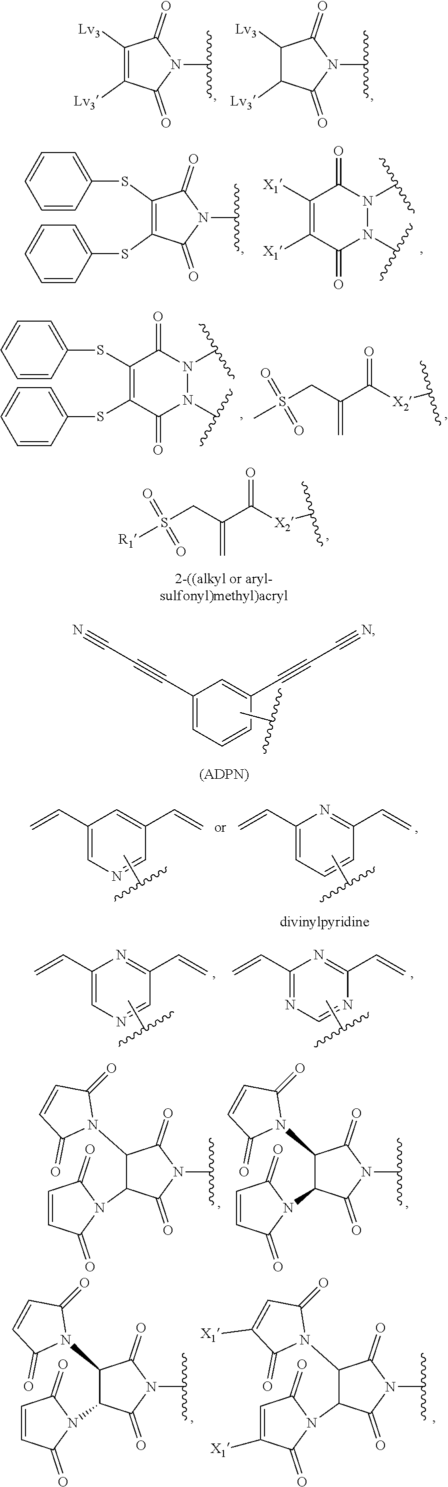 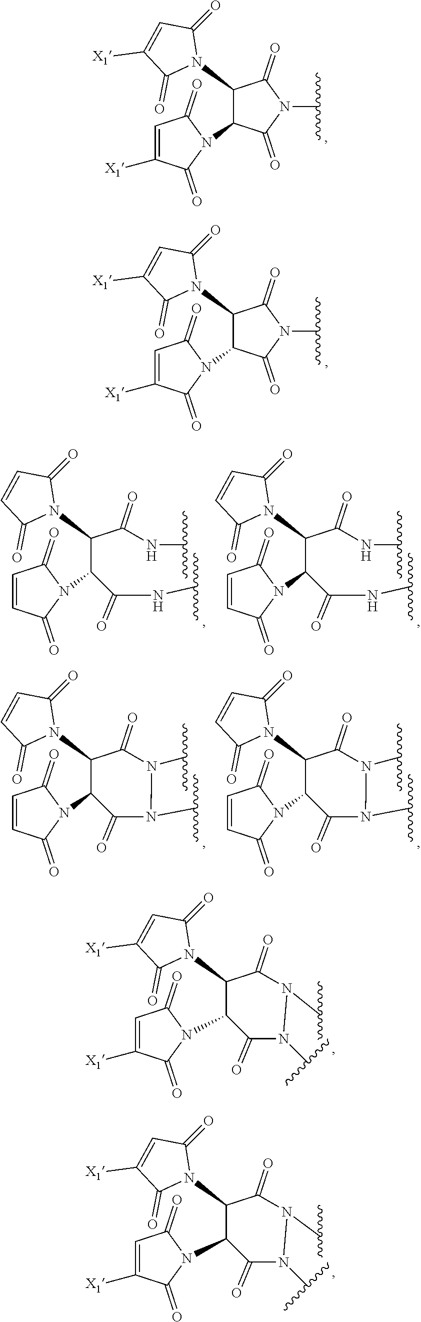 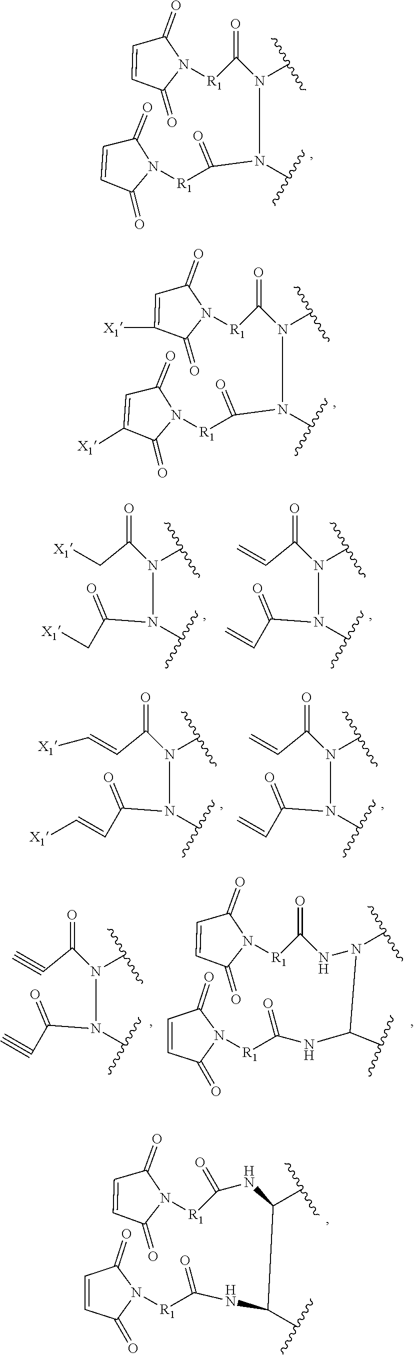 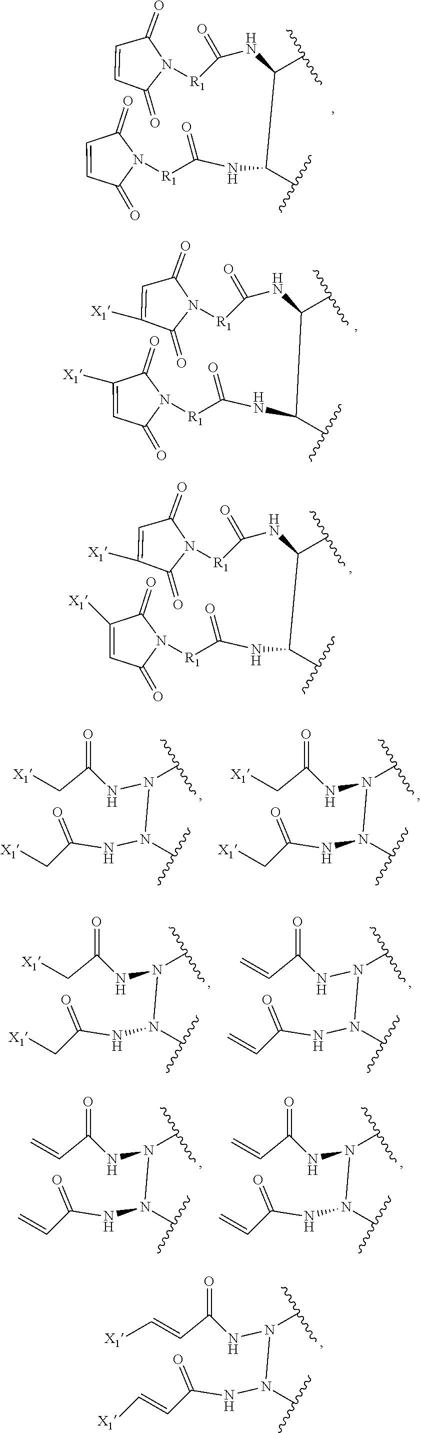 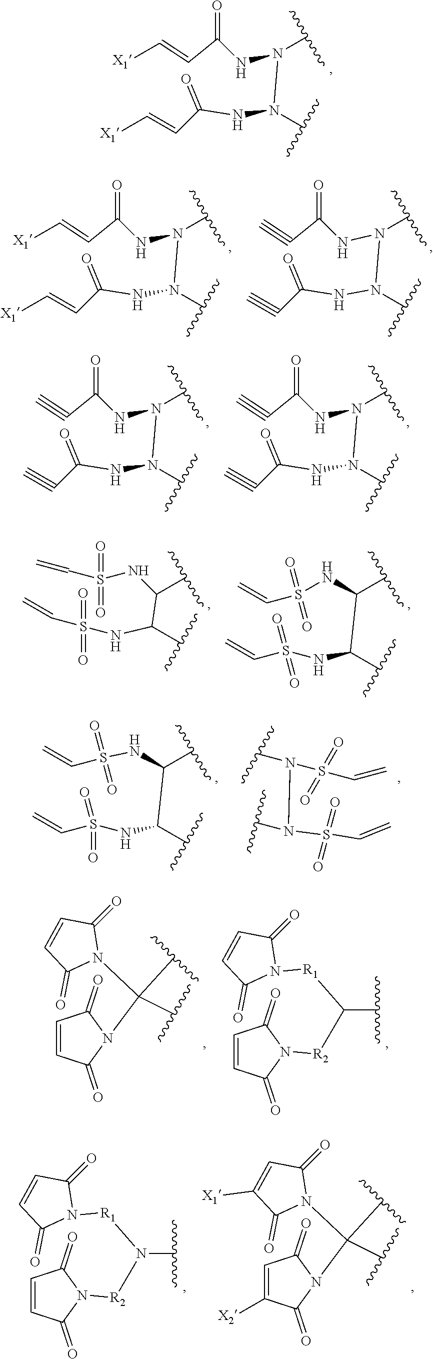 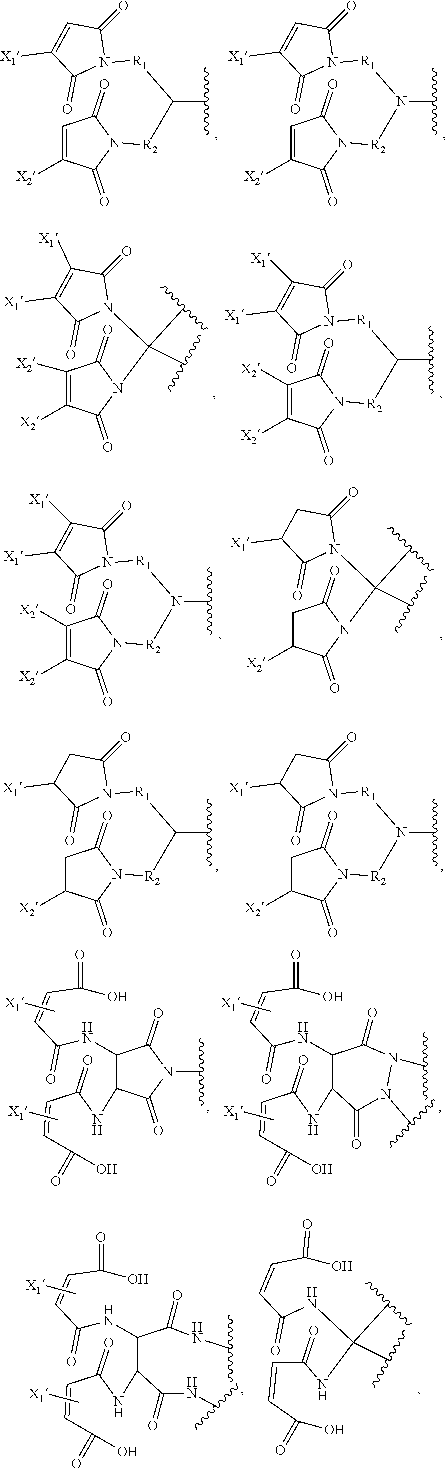 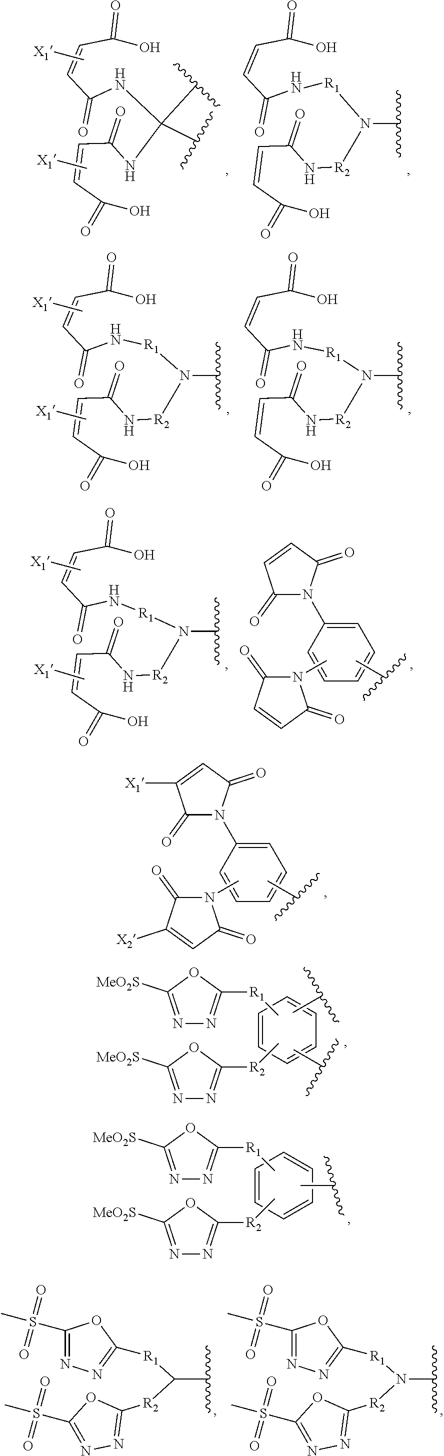 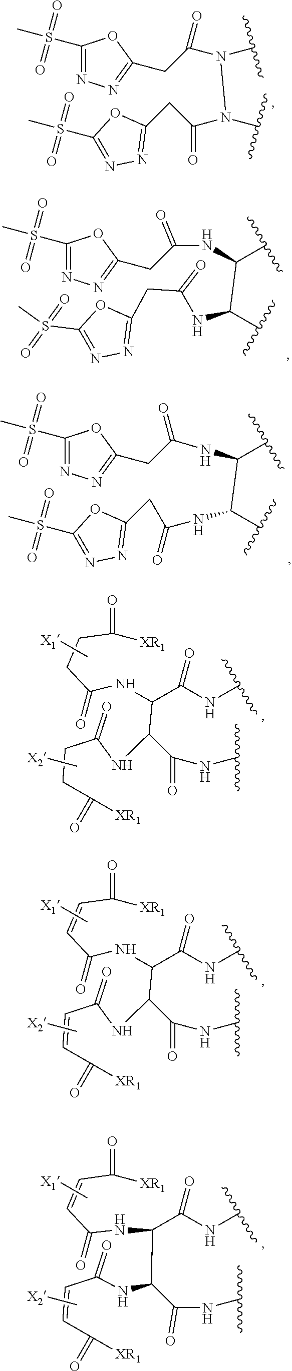 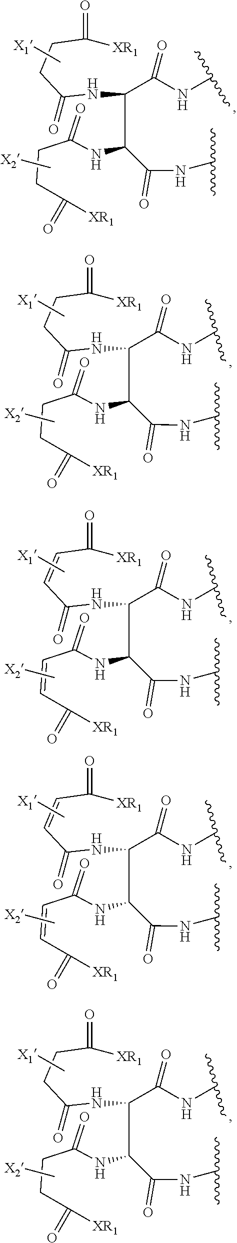 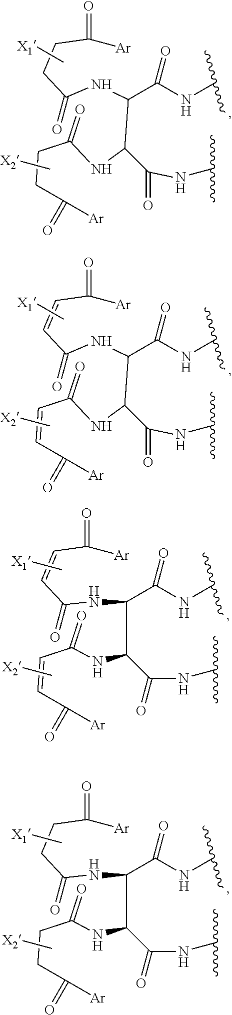 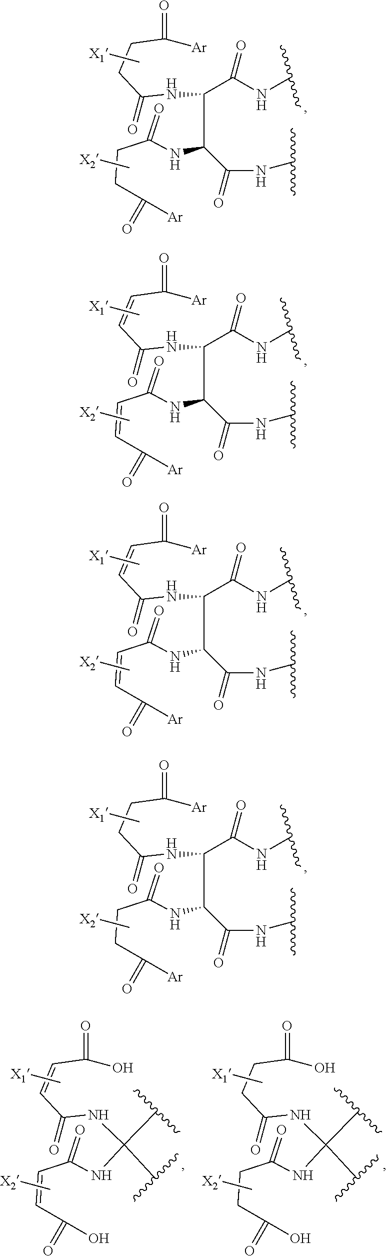 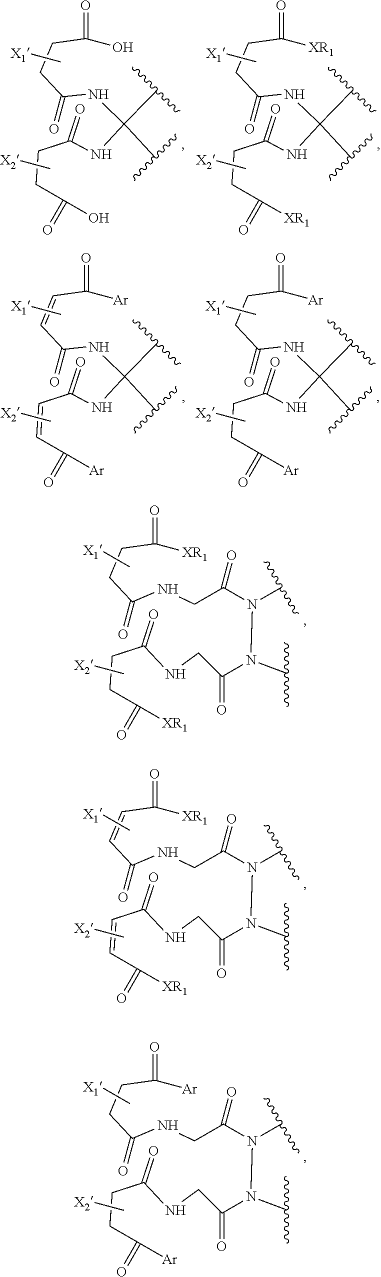 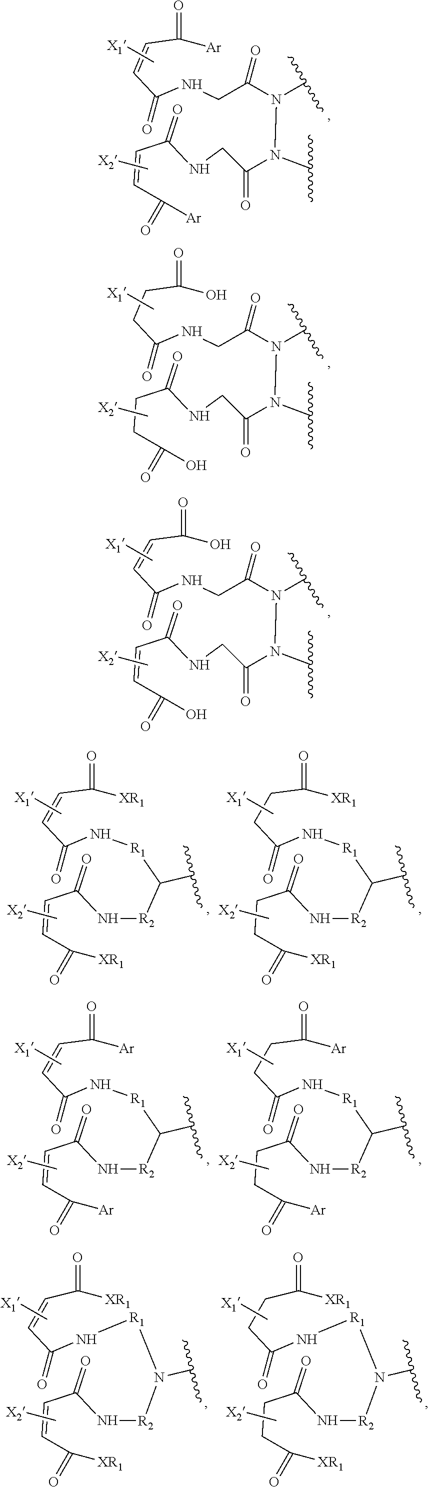 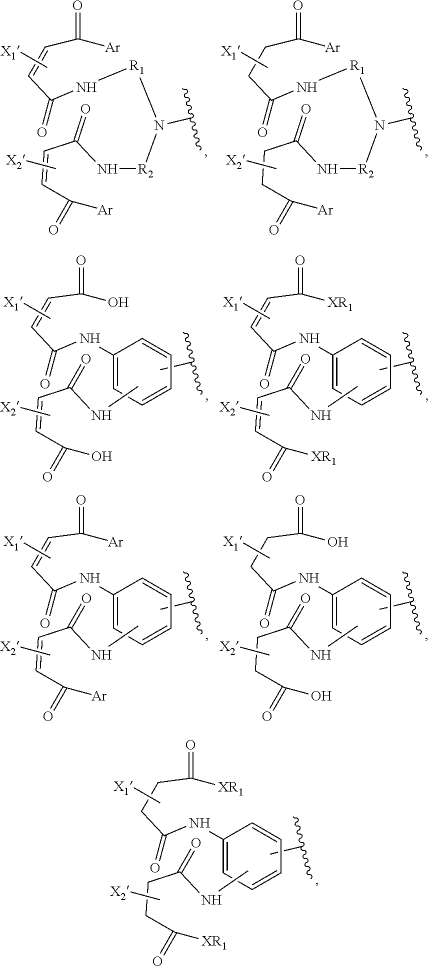 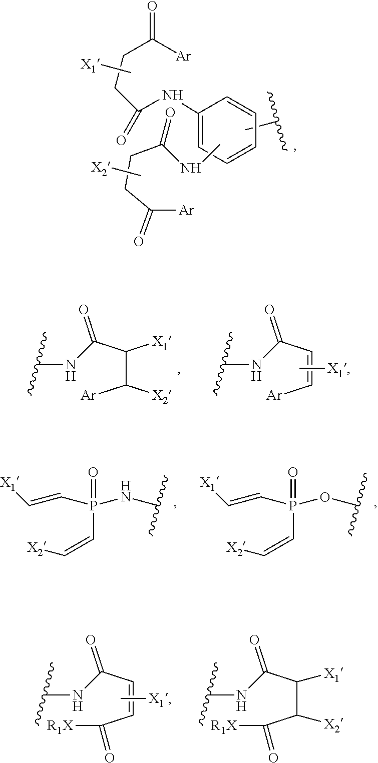
wherein Lv₃, Lv₃′, X₁′ and X₂′ are described the same above; the connecting bond “ [symbol] ” in the middle of the two atoms means it can link either one of the two atoms.

47. A method for preparing the antibody-drug conjugate of claim 40 comprising reacting a linker compound having formula (VII), (VIII) or (IX) illustrated below first to a cytotoxic drug to form a compound of formula (IV), (V) or (VI), followed by reaction to an amino acid in the antibody to form the conjugate of formula (I), (II), or (III): 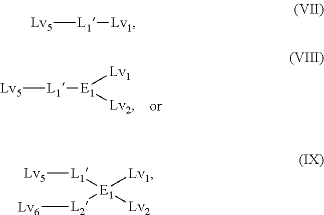
wherein L₁′, L₂′, E₁, Lv₁, and Lv₂ are defined the same above for Formula (I), (II) (III). (IV), (V), and (VI); wherein Lv₅ and Lv₆ are independently selected from: 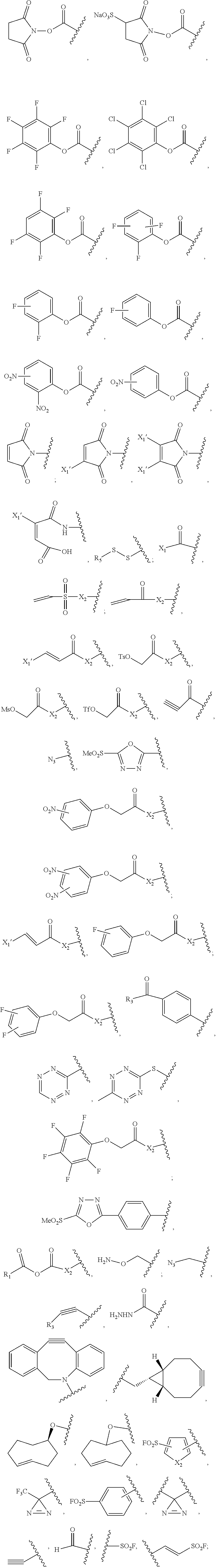
wherein X₁′ is F, Cl, Br, I, Ots (tosylate), OTf (triflate), Oms (mesylate), OC₆H₄(NO₂), OC₆H₃(NO₂)₂, OC₆F₅, OC₆HF₄, or Lv₃; X₂′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions; wherein the function groups Lv₅ and/or Lv₆ can be also reacted with a thiol in a cytotoxic drug as long as the reaction are at least one fold faster or slower than the reaction between Lv₁ or Lv₂ and a thiol in an antibody.

48. A method for preparing the antibody-drug conjugate of claim 40 comprising generating thiols in the antibody by reduction of disulfide bonds, then reacting the thiols simultaneously or sequentially to a linker of formula (VII), (VIII) or (IX) to form the antibody/linker complex molecule of formula (X), (XI) or (XII) below, following by reaction with a cytotoxic drug D₁ or D2 independently to form the conjugate of formula (I), (II), or (III): 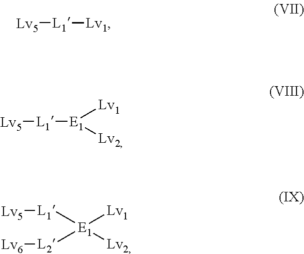
wherein L₁′, L₂′, E₁, Lv₁, and Lv₂ are defined the same above for Formula (I), (II) (III). (IV), (V), and (VI); wherein Lv₅ and Lv₆ are independently selected from: 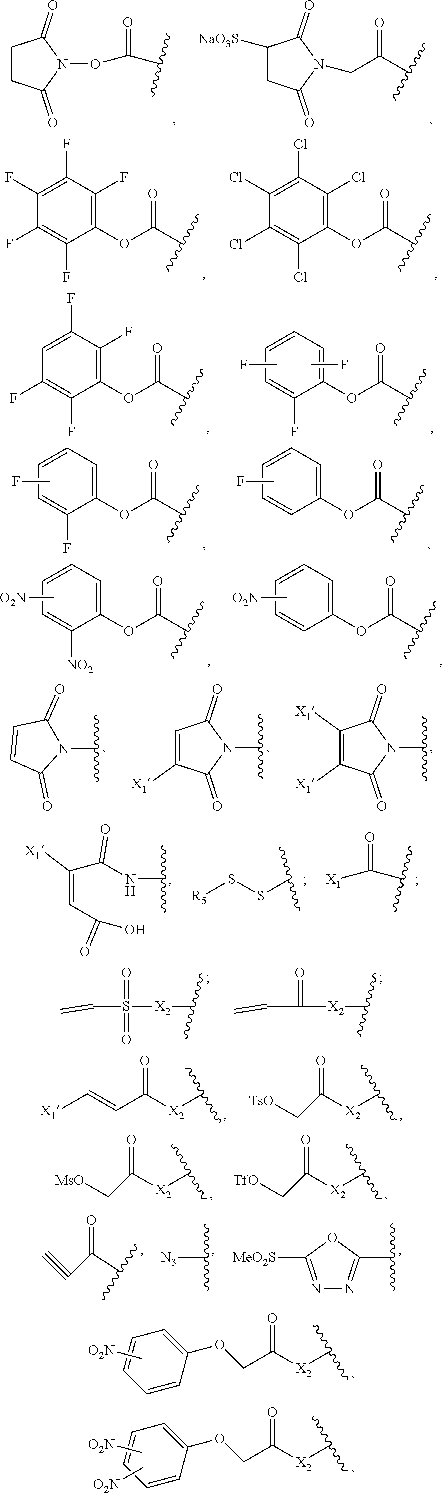 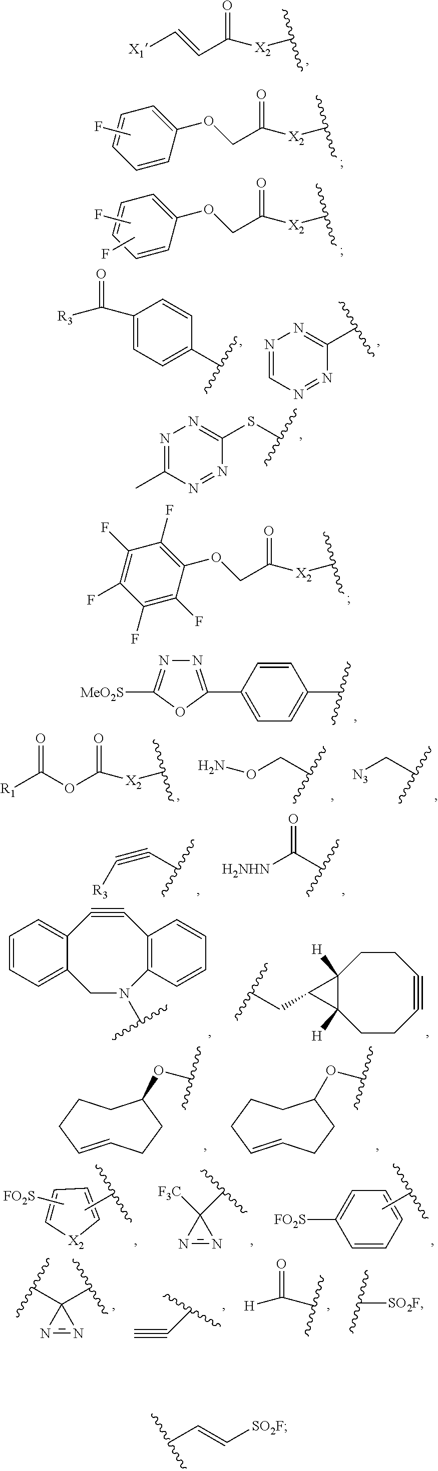
wherein X₁′ is F, Cl, Br, I, Ots (tosylate), OTf (triflate), Oms (mesylate), OC₆H₄(NO₂), OC₆H₃(NO₂)₂, OC₆F₅, OC₆HF₄, or Lv₃; X₂′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions; wherein the function groups Lv₅ and/or Lv₆ can be also reacted with a thiol in a cytotoxic drug as long as the reaction are at least one fold faster or slower than the reaction between Lv₁ or Lv₂ and a thiol in an antibody; 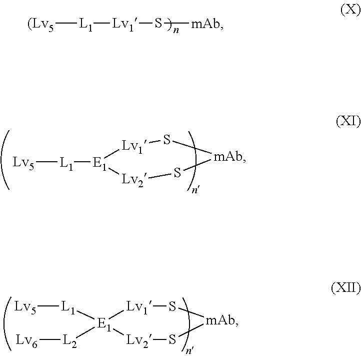
wherein Lv₅, Lv₆, L₁, L₂, E₁, Lv₁′ Lv₂′, mAb, n and n′ are the same as described above.

49. A method for preparing the antibody-drug conjugate of claim 40 comprising a homogenous conjugation process which comprises the following steps:
(a) incubating the antibody in presence of an effective amount of Zinc cation-amino chelate/complex (Zn(NR₁R₂R₃)ₘ₁²⁺ and a reductant, in a buffer system to selectively reduce inter-chain disulfide bonds within the antibody;
(b). introducing an effective amount of payload/linker complex/assembly of formula (IV), (V) or (VI) or linker of formula (VII), (VIII) or (IX) to react with the thiol groups resulted from step (a); 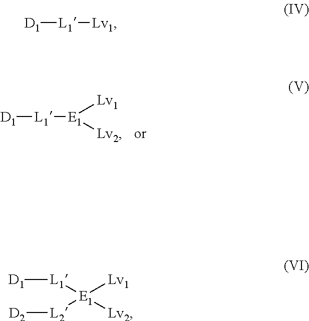
wherein: Lv₁ and Lv₂ are a reactive group, and are independently selected from: 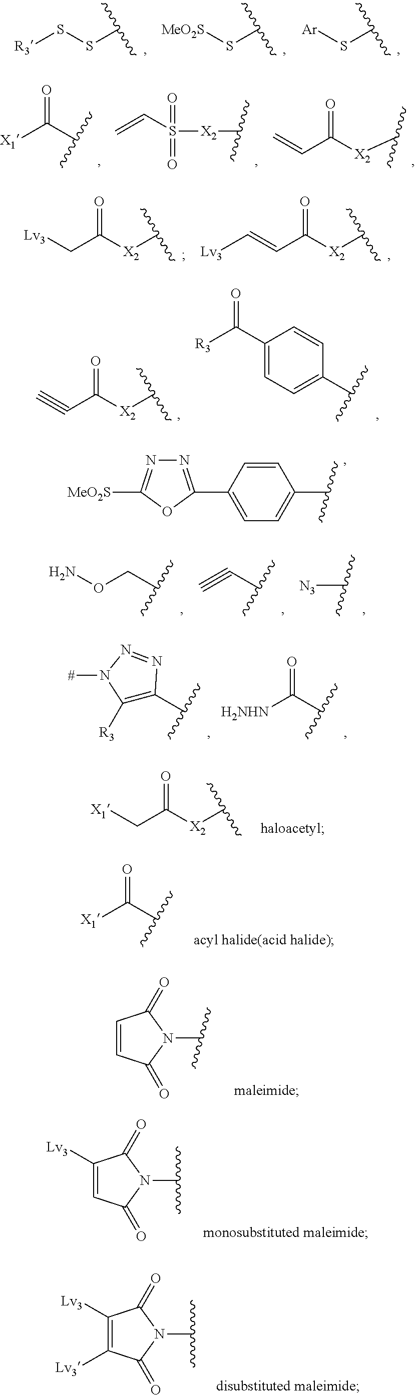 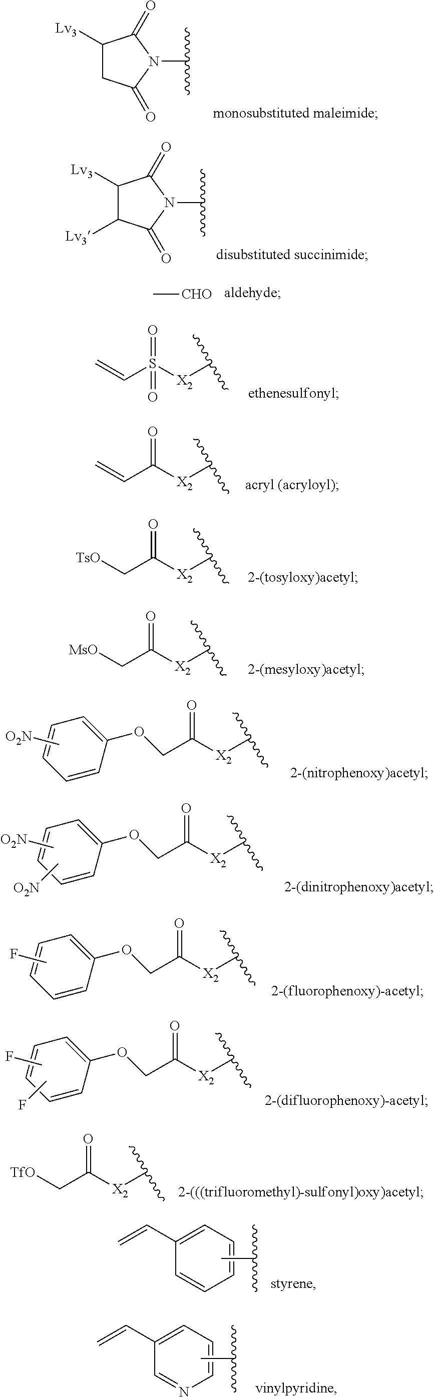 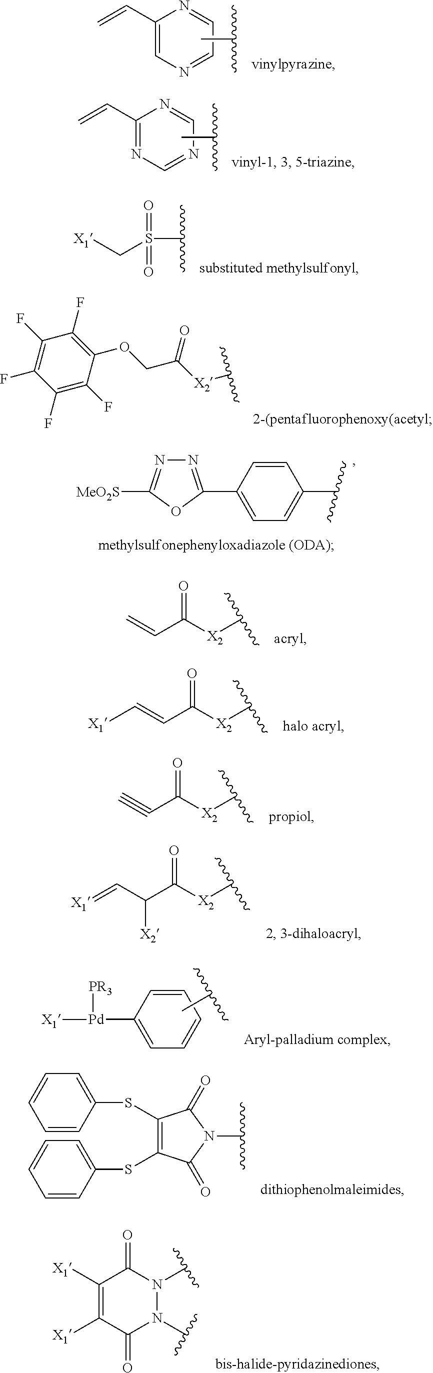 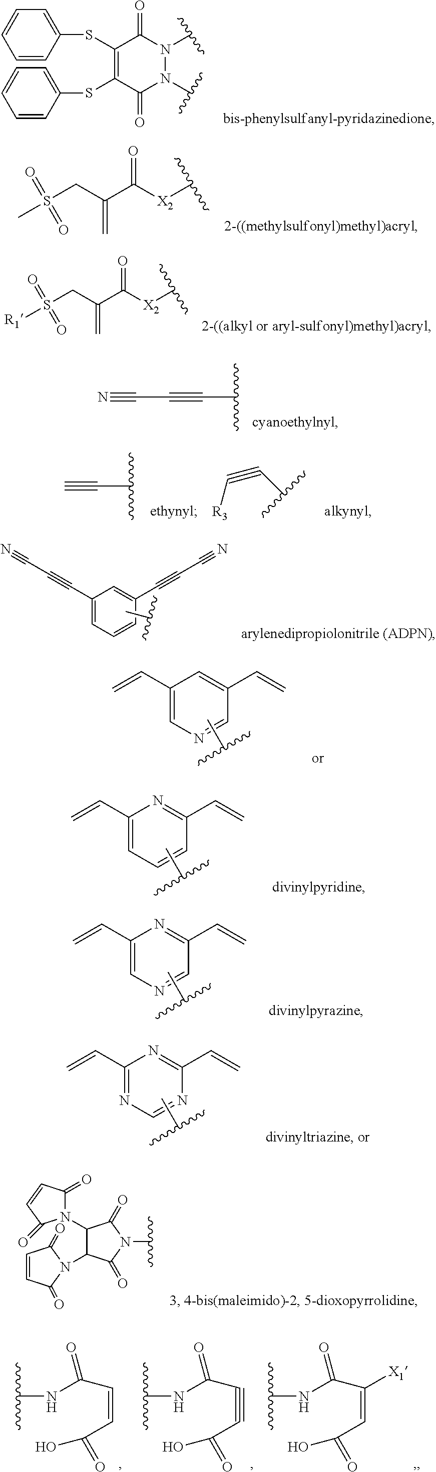 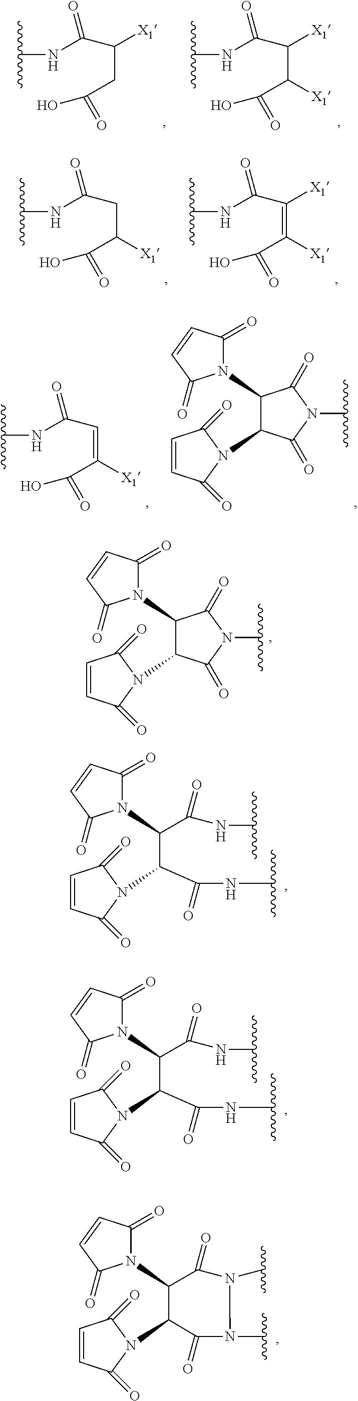 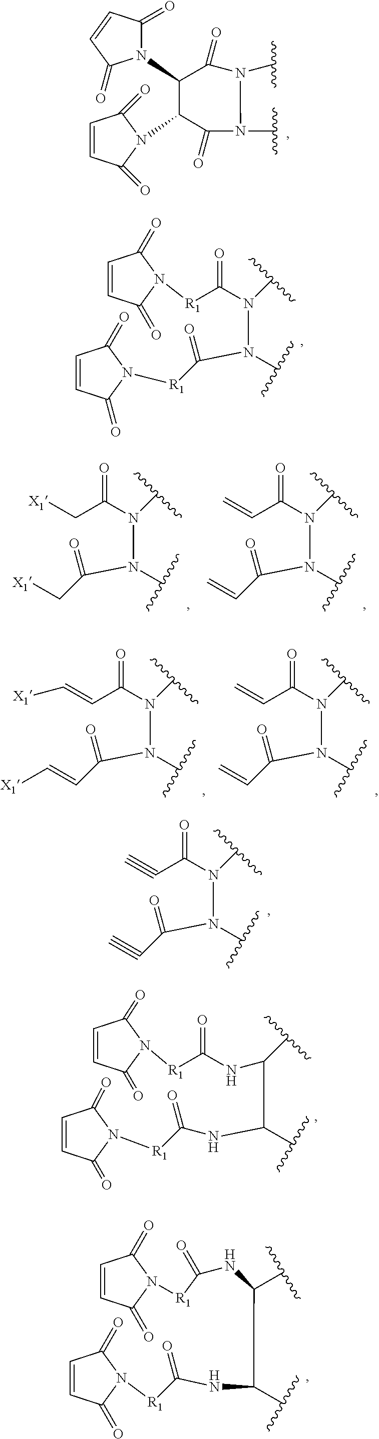 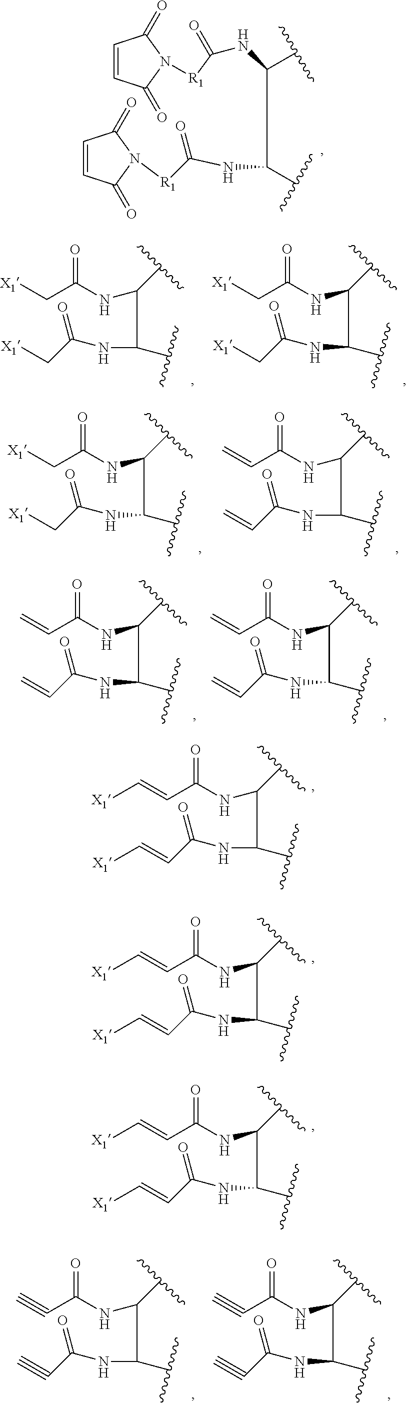 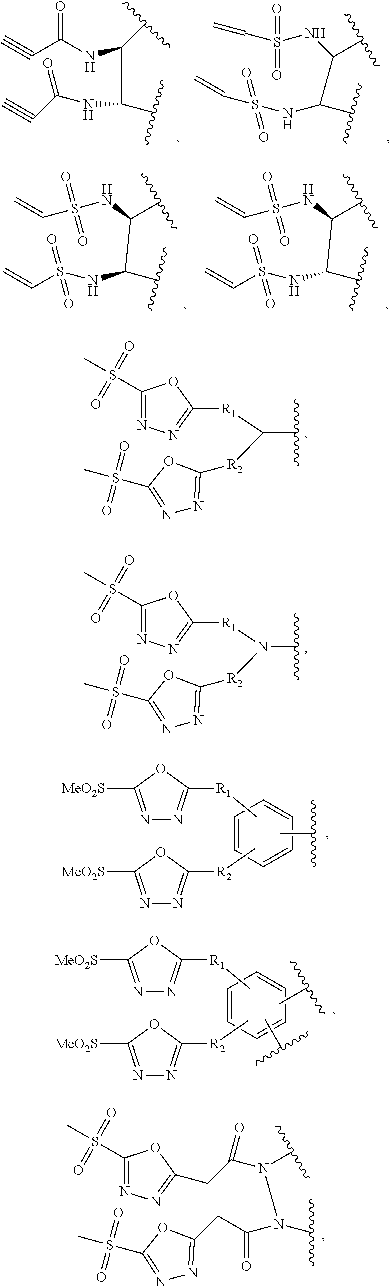 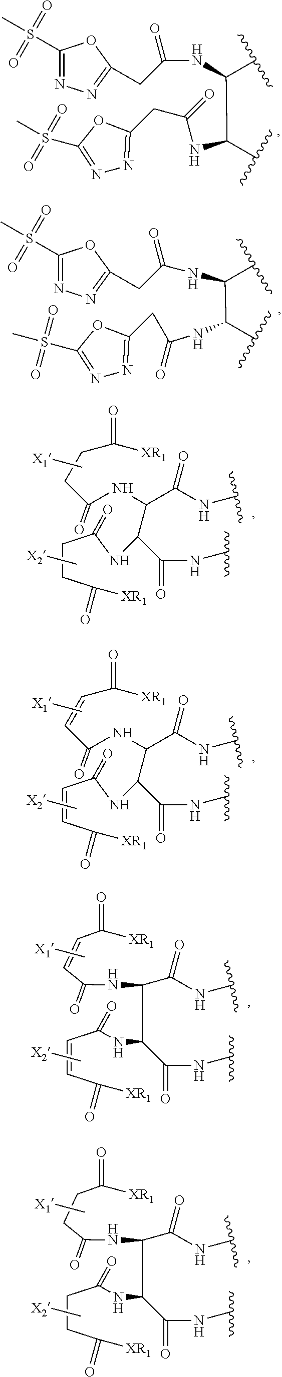 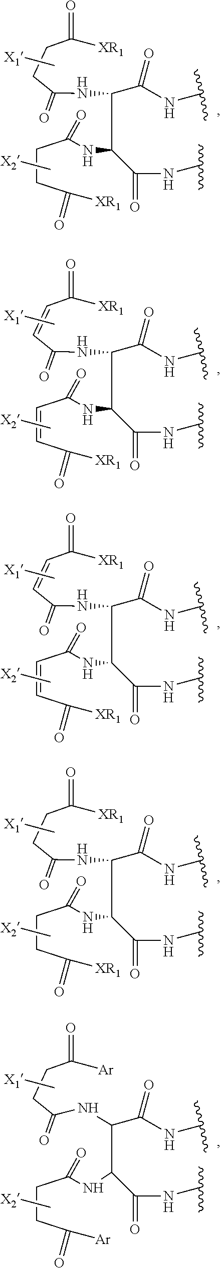 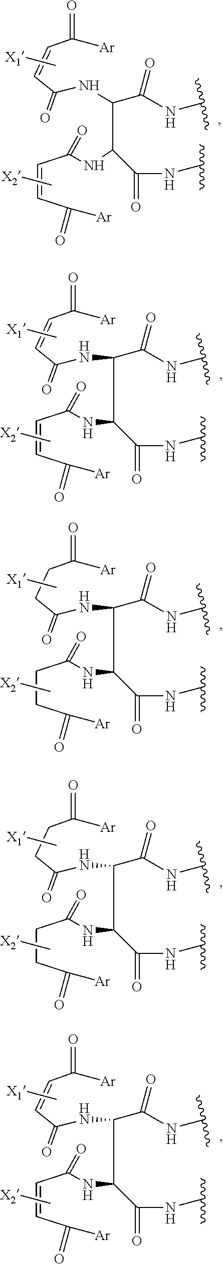 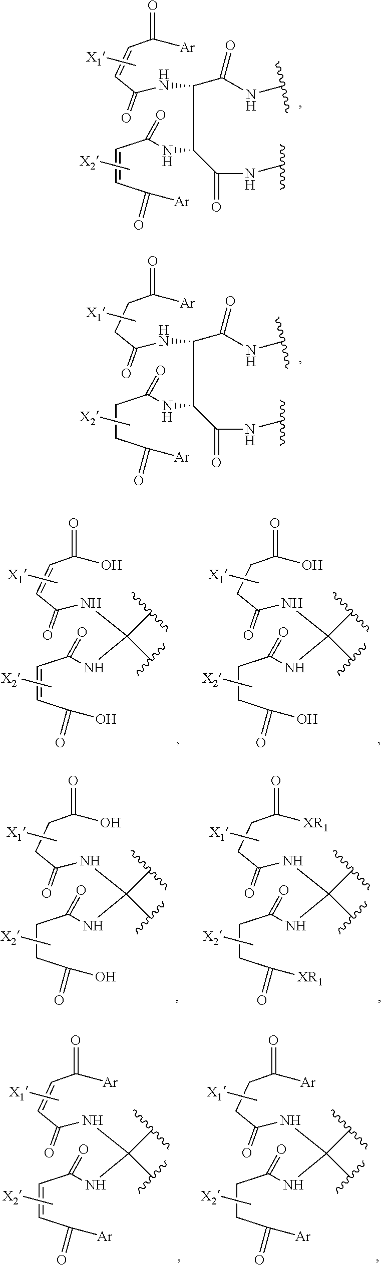 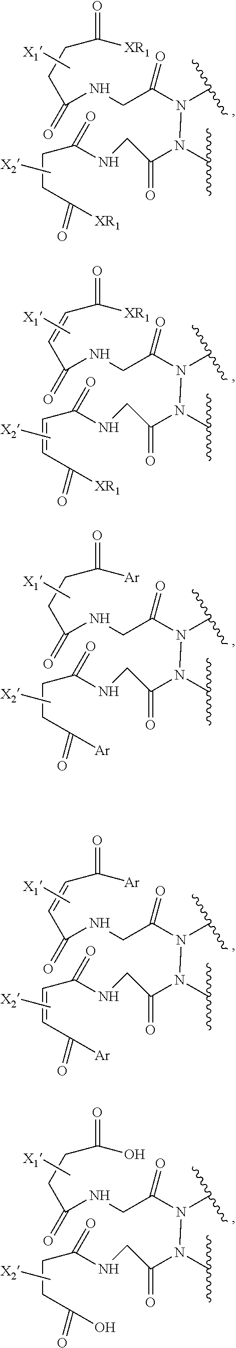 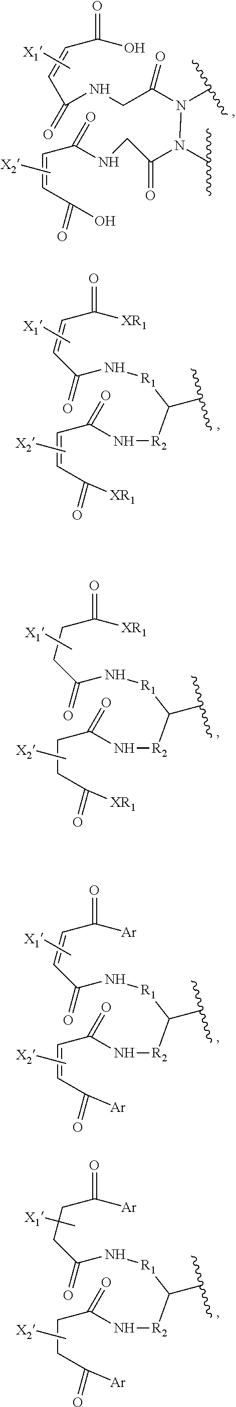 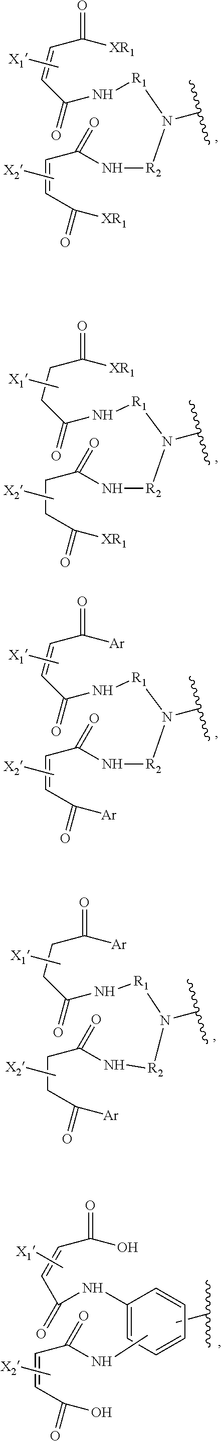 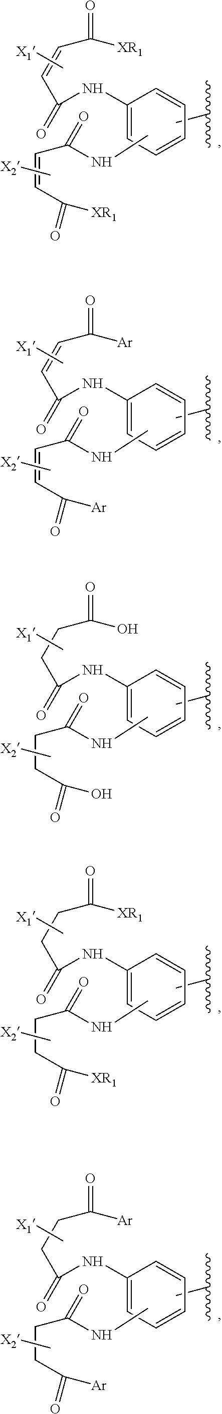 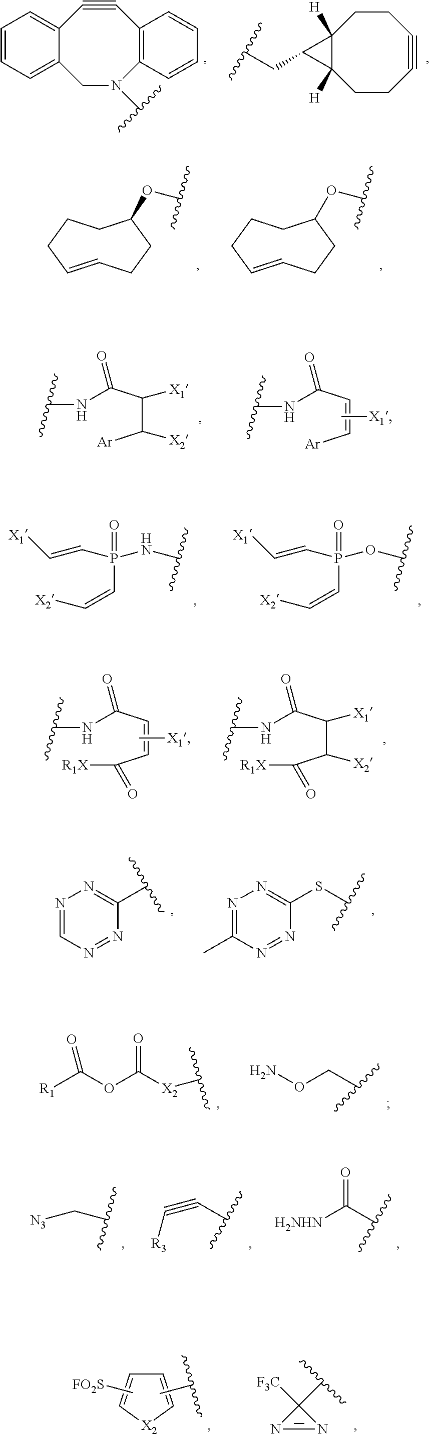 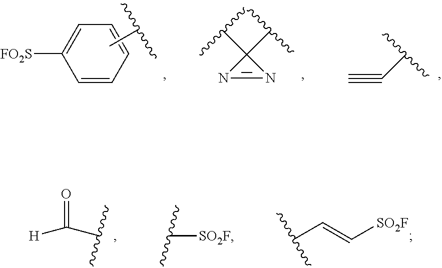
wherein X₁′ and X₂′ are independently F, Cl, Br, I, OTf, Oms, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;
L₁′ and L₂′ are, the same or different, independently selected from O, NH, S, S—S, NHNH, N(R₃), N(R₃)N(R_{3′}), C₁-C₈ of alkyl; C₂-C₈ of heteroalkyl, alkylcycloalkyl, or heterocycloalkyl; C₃-C₈ of aryl, Ar-alkyl, heterocyclic, carbocyclic, cycloalkyl, heteroalkylcycloalkyl, alkylcarbonyl, or heteroaryl; C₂-C₈ (2-8 carbon atoms) of ester, ether, or amide; 1˜8 natural or unnatural amino acids described in the definition; polyethyleneoxy unit of formula (OCH₂CH₂)ₚ, (OCH₂CH(CH₃))ₚ, (OCH₂CH₂)ₚOR₃, (OCH₂CH(CH₃))ₚOR₃, NH(CH₂CH₂O)ₚR₃, NH(CH₂CH(CH₃)O)ₚR₃, N[(CH₂CH₂—O)ₚR₃][(CH₂CH₂O)ₚR₃′], (OCH₂CH₂)ₚCOOR₃, or CH₂CH₂(OCH₂CH₂)ₚCOOR₃, wherein p and p′ are independently an integer selected from 0 to about 1000, or combination thereof, wherein R₃ and R₃′ are independently H; C₁-C₈ of alkyl; C₂-C₈ of heteroalkyl, alkylcycloalkyl, or heterocycloalkyl; C₃-C₈ of aryl, Ar-alkyl, heterocyclic, cycloalkyl, heteroalkylcycloalkyl, alkylcarbonyl, or heteroaryl;
L₁′ and/or L₂′ may contain a self-immolative or a non-self-immolative component, peptidyl units, a hydrazone bond, a disulfide, an ester, an oxime, an amide, or a thioether bond;
wherein the components of 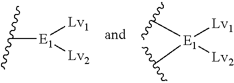
in the formula (V) and formula (VI) are independently selected from: 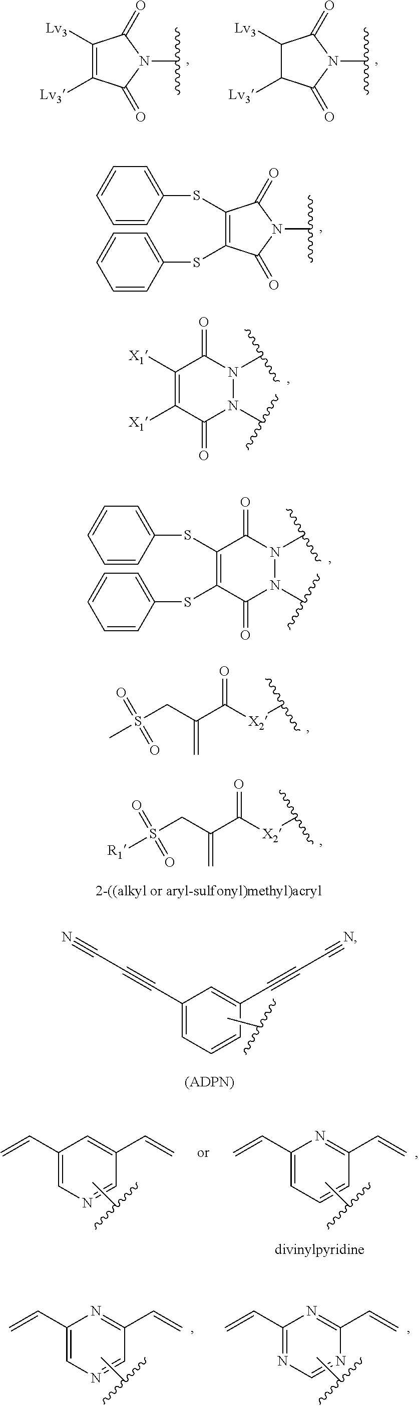 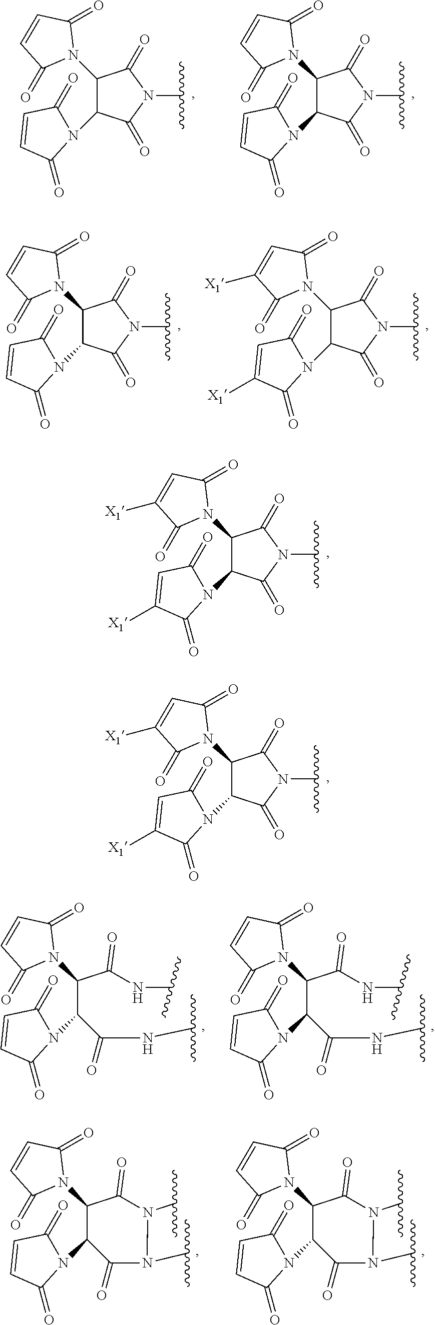 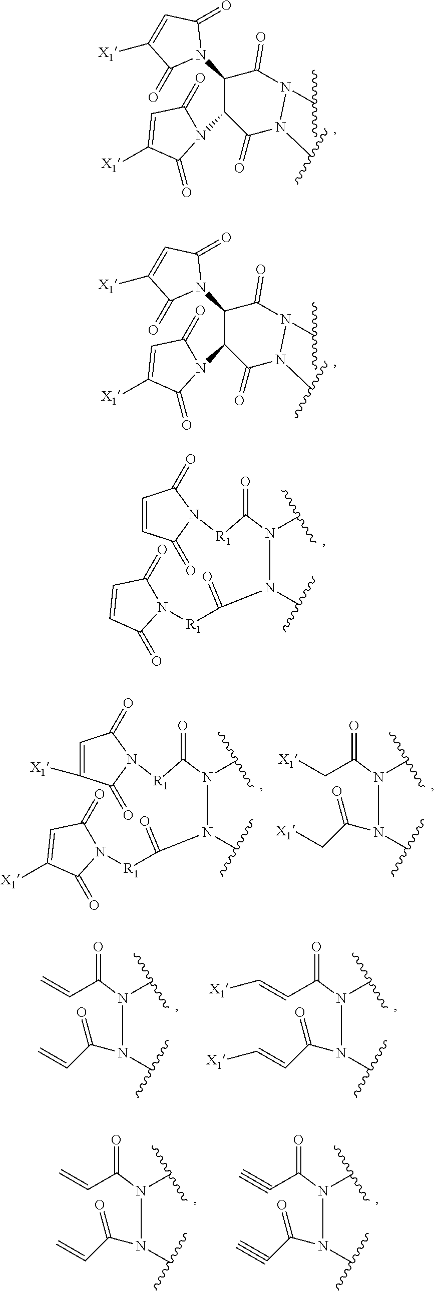 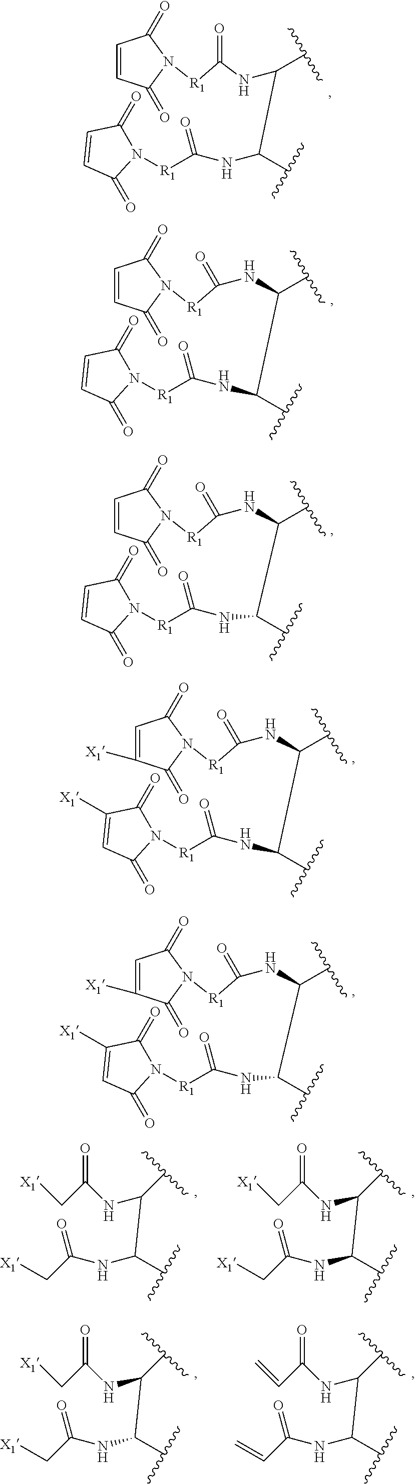 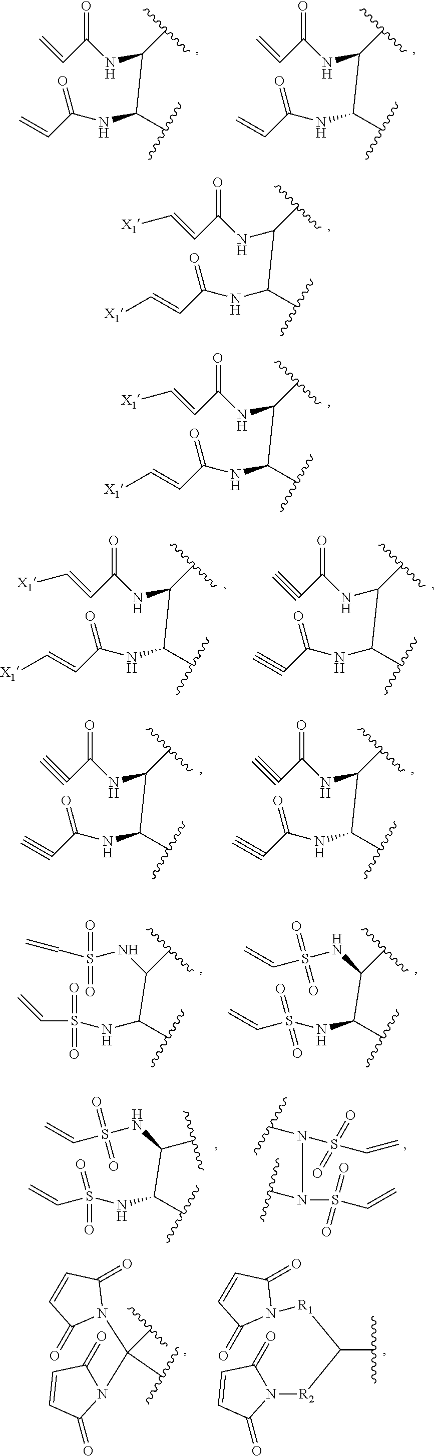 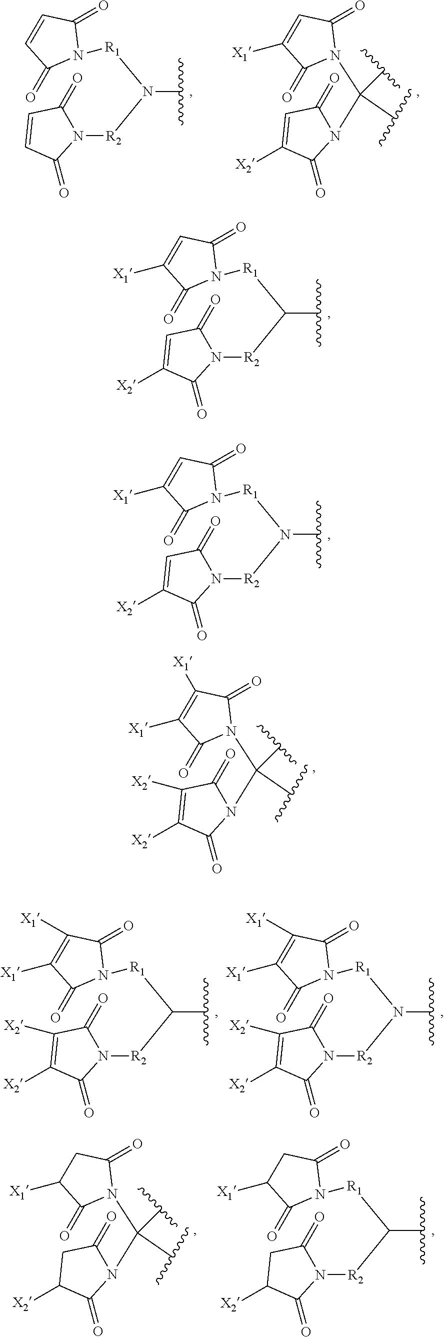 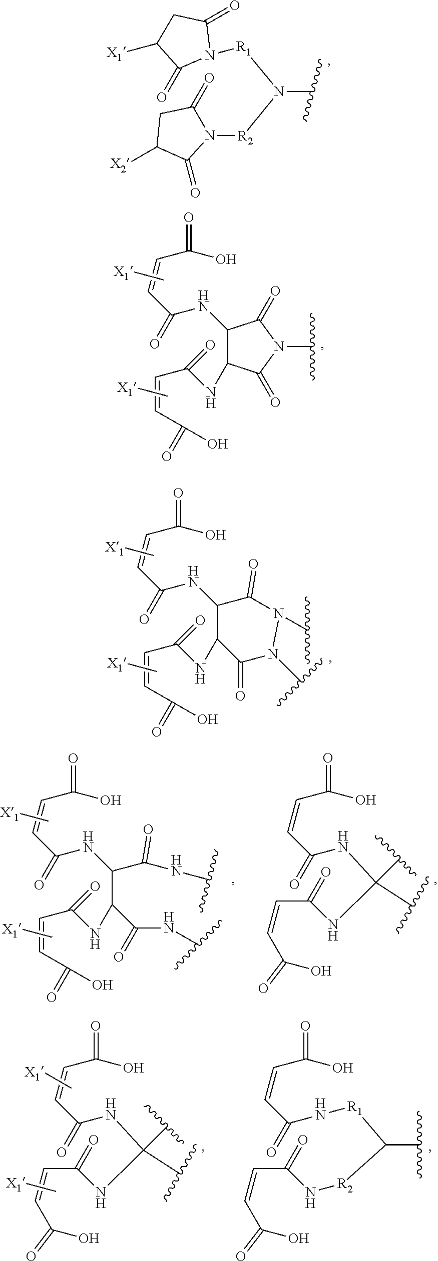 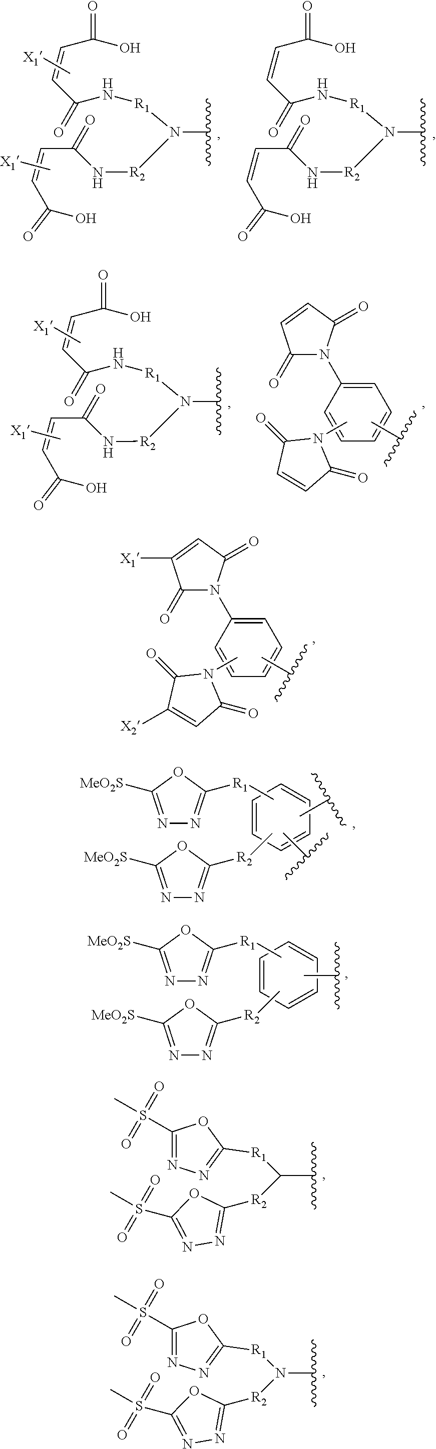 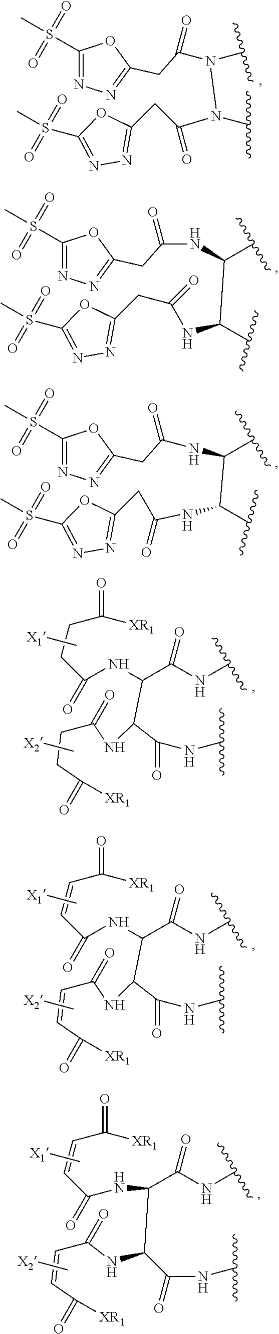 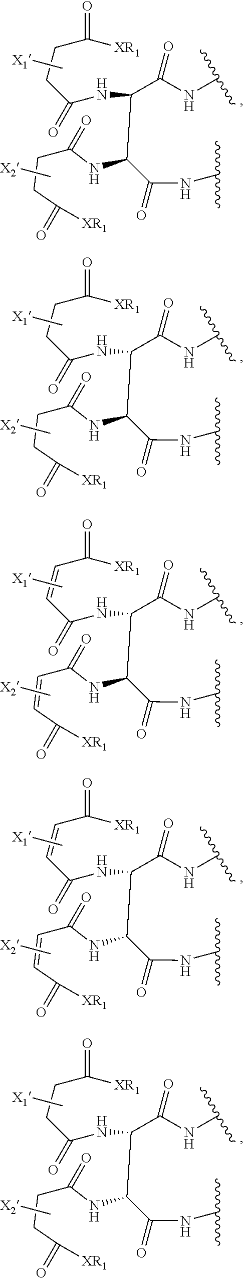 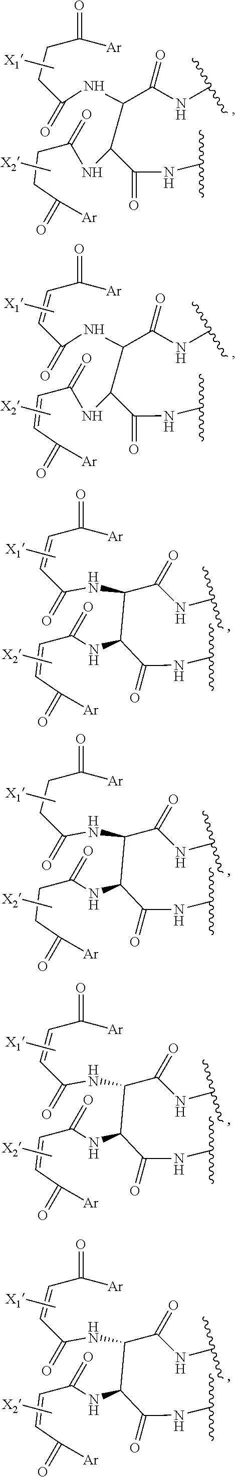 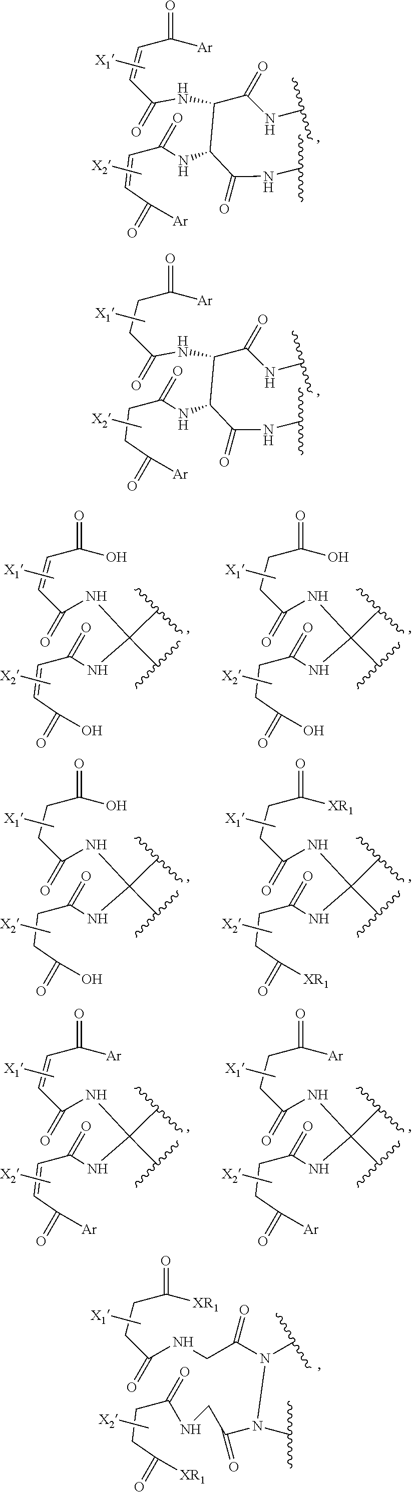 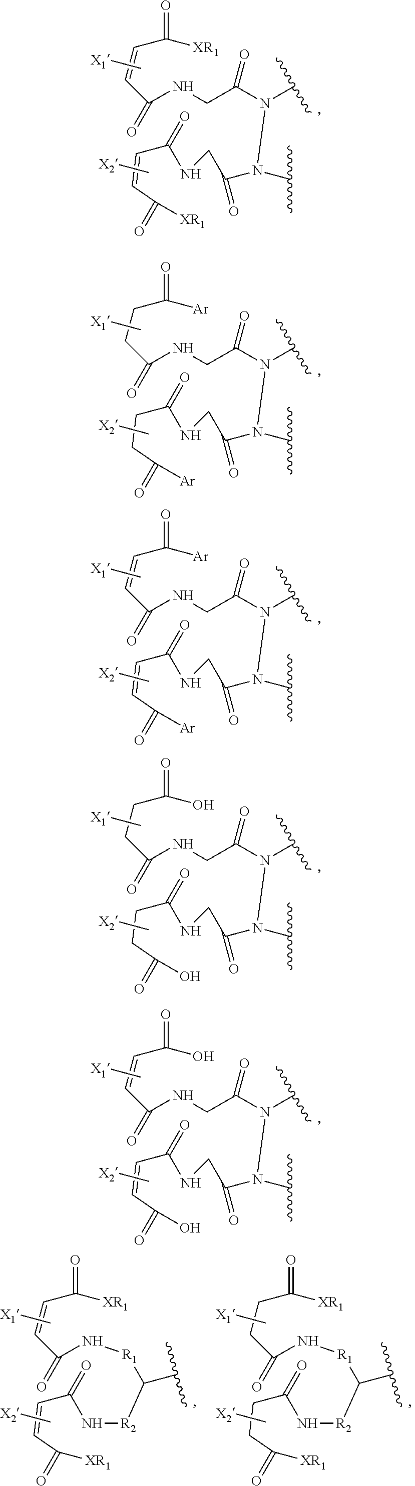 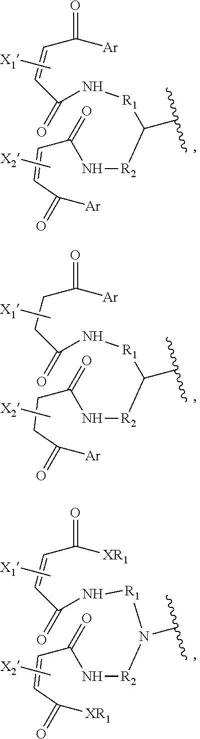 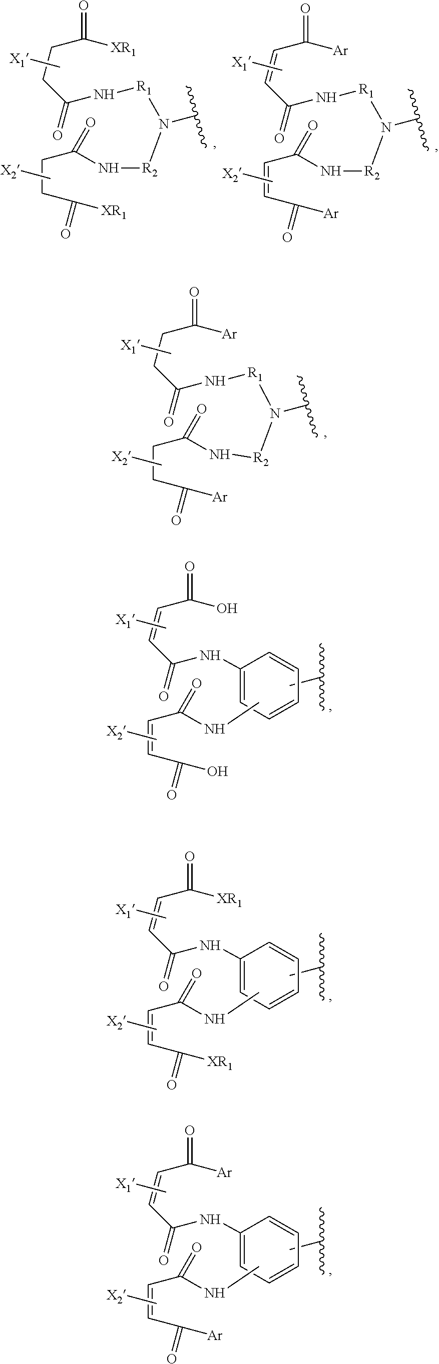 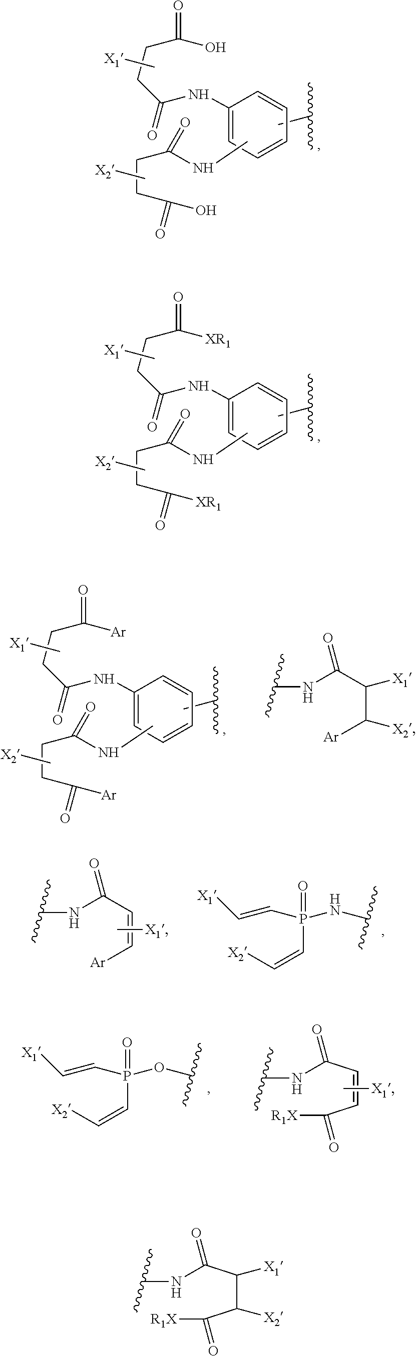
wherein Lv₃, Lv₃′, X₁′ and X₂′ are described the same above; the connecting bond “ [symbol] ” in the middle of the two atoms means it can link either one of the two atoms; 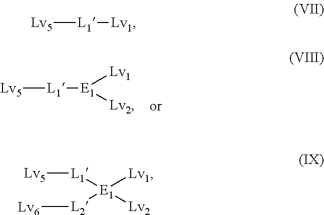
wherein L₁′, L₂′, E₁, Lv₁, and Lv₂ are defined the same above for Formula (I), (II) (III). (IV), (V), and (VI); wherein Lv₅ and Lv₆ are independently selected from: 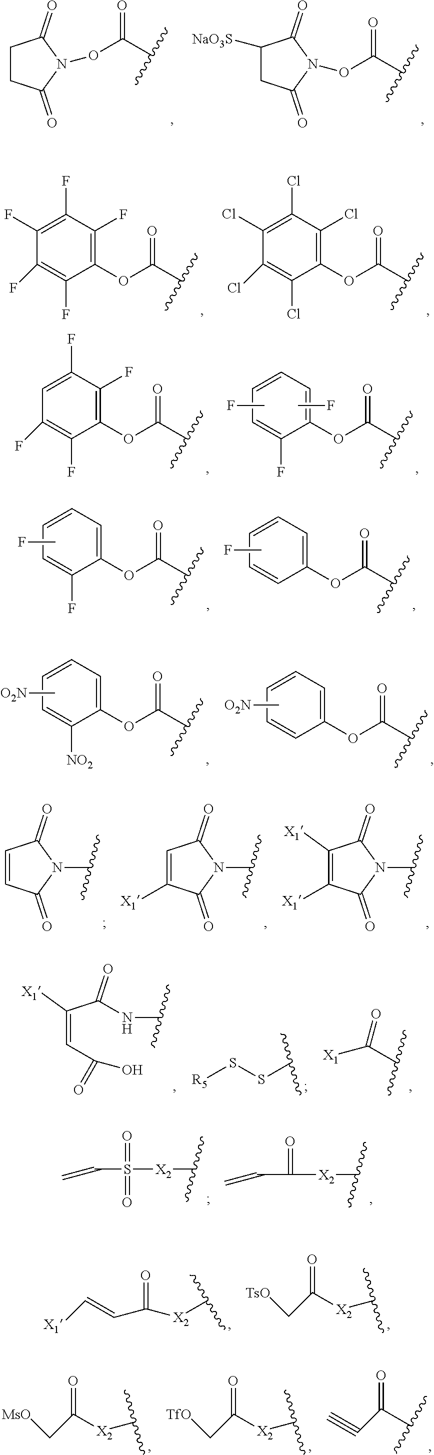 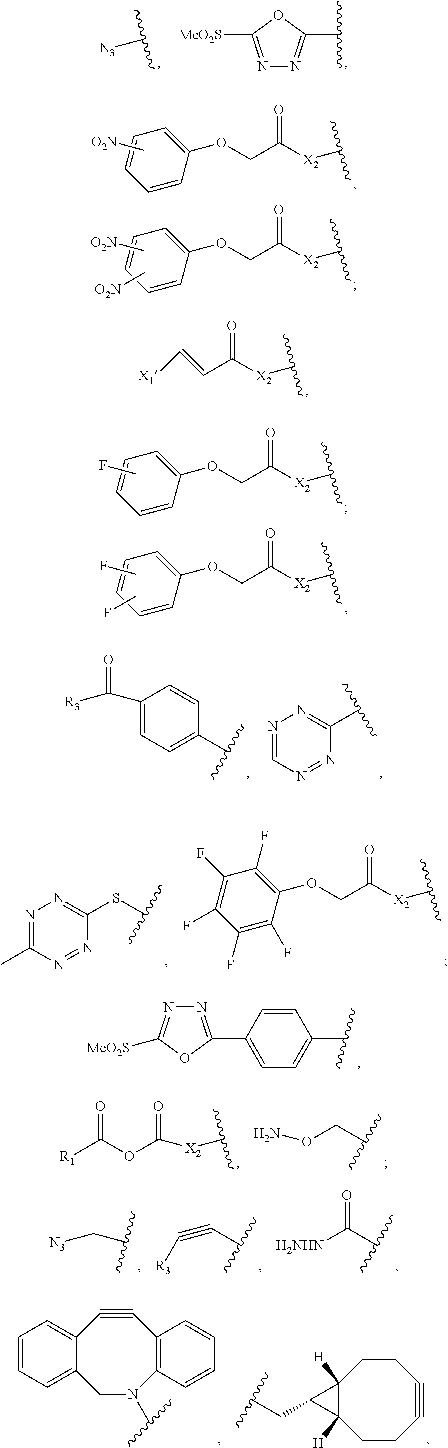 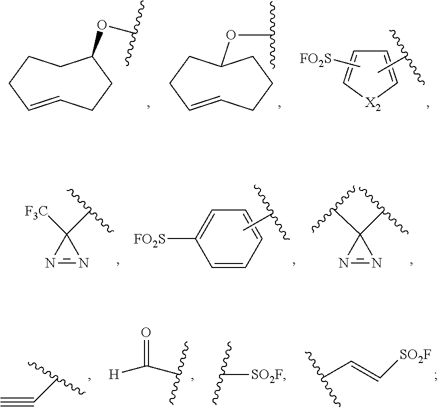
wherein X₁′ is F, Cl, Br, I, Ots (tosylate), Otf (triflate), Oms (mesylate), OC₆H₄(NO₂), OC₆H₃(NO₂)₂, OC₆F₅, OC₆HF₄, or Lv₃; X₂′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions; wherein the function groups Lv₅ and/or Lv₆ can be also reacted with a thiol in a cytotoxic drug as long as the reaction are at least one fold faster or slower than the reaction between Lv₁ or Lv₂ and a thiol in an antibody; and
(c). optionally adding an effective amount of oxidant (dehydroascorbic acid) to re-oxidize unreacted thiol groups;
(d). and purifying the resulted conjugate;
wherein the optional step (c) can be optionally replaced by: adding an effective amount of cystine or relative disulfide compounds or 4-(azidomethyl)benzoic acid to quench the unreacted reductant, while generating cysteine from the reduction of the cystine to quench the excessive conjugation linker or linker/payload complex containing a thiol reactive group; an effective amount of cysteine or relative thiol compounds can be also added to quench the excessive linker or linker/payload complex molecule;
wherein R₁, R₂ and R₃ in the formula of the Zinc cation-amino chelate/complex Zn(NR₁R₂R₃)ₘ₁²⁺ are independently selected from C₁-C₈ of alkyl; C₂-C₈ of heteroalkyl, alkylcycloalkyl, or heterocycloalkyl; C₃-C₈ of aryl, Ar-alkyl, heterocyclic, carbocyclic, cycloalkyl, heteroalkylcycloalkyl, alkylcarbonyl, or heteroaryl; m1 is 1, 2, 3 or 4;
wherein the (Zn(NR₁R₂R₃)m₁²⁺ is selected from, Zn(NH₂CH₃)₂²⁺, Zn(NH₂CH₂CH₃)₂²⁺, Zn(NH₂CH₂CH₂CH₃)₂²⁺, Zn(NH₂CH(CH₃)₂)₂²⁺, Zn(NH₂C(CH₃)₃)₂²⁺, Zn(NH₂CH₂C(CH₃)₃)₂²⁺, Zn(NH(CH₃)₂)₂²⁺, Zn(NH(CH₂CH₃)₂)₂²⁺, Zn(NH(CH(CH₃)₂)₂)₂²⁺, Zn(NH(C(CH₃)₃)₂)₂²⁺, Zn(NH(CH(CH₂CH₃)₂)₂)₂²⁺, Zn(NH(CH₂C(CH₃)₃)₂)₂²⁺, Zn(NH(CH₂C(CH₂CH₃)₃)₂)₂²⁺, Zn(NH(CH₂CH₂C(CH₃)₃)₂)₂²⁺, Zn(NH₂CH₂CH₂OH)₂²⁺, Zn(NH(CH₂CH₂OH)₂)₂²⁺, Zn(N(CH₂CH₂OH)₃)₂²⁺, Zn(NH₂CH₂COOH)₂²⁺, Zn(NH₂CH₂CONH₂)₂²⁺, Zn(NH₂CH₂COOCH₃)₂²⁺, Zn(NH₂CH₂COOCH₂CH₃)₂²⁺, Zn(NH₂CH₂COOC(CH₃)₃)₂²⁺, Zn(NH₂CH₂COOCH(CH₃)₂)₂²⁺, Zn(NH₂CH₂CH₂COOH)₂²⁺, Zn(NH(CH₂COOH)₂)₂²⁺, Zn(N(CH₂CH₂COOH)₃)₂²⁺, Zn(NH₂CH₃)₄²⁺, Zn(NH₂CH₂CH₃)₄²⁺, Zn(NH₂CH₂CH₂CH₃)₄²⁺, Zn(NH₂CH(CH₃)₂)₄²⁺, Zn(NH₂C(CH₃)₃)₄²⁺, Zn(NH₂CH₂C(CH₃)₃)₄²⁺, Zn(NH(CH₃)₂)₄²⁺, Zn(NH(CH₂CH₃)₂)₄²⁺, Zn(NH(CH(CH₃)₂)₂)₄²⁺, Zn(NH(C(CH₃)₃)₂)₄²⁺, Zn(NH(CH(CH₂CH₃)₂)₂)₄²⁺, Zn(NH(CH₂C(CH₃)₃)₂)₄²⁺, Zn(NH(CH₂C(CH₂CH₃)₃)₂)₄²⁺, Zn(NH(CH₂CH₂C(CH₃)₃)₂)₄²⁺, Zn(NH₂CH₂CH₂OH)₄²⁺, Zn(NH(CH₂CH₂OH)₂)₄²⁺, Zn(N(CH₂CH₂OH)₃)₄²⁺, Zn(NH₂CH₂COOH)₄²⁺, Zn(NH₂CH₂CONH₂)₄²⁺, Zn(NH₂CH₂COOCH₃)₄²⁺, Zn(NH₂CH₂COOCH₂CH₃)₄²⁺, Zn(NH₂CH₂COOC(CH₃)₃)₄²⁺, Zn(NH₂CH₂COOCH(CH₃)₂)₄²⁺, Zn(NH₂CH₂CH₂COOH)₄²⁺, Zn(NH(CH₂COOH)₂)₄²⁺, Zn(N(CH₂CH₂COOH)₃)₄²⁺, 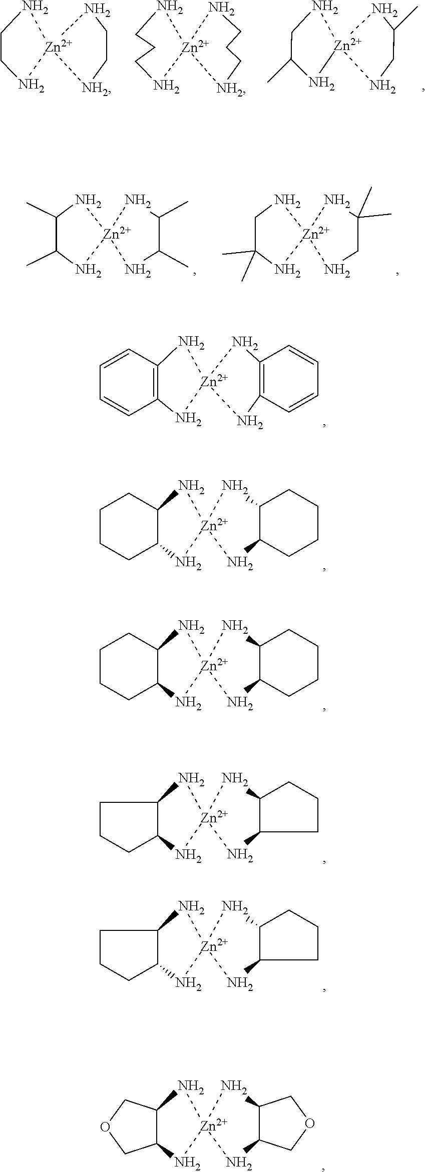 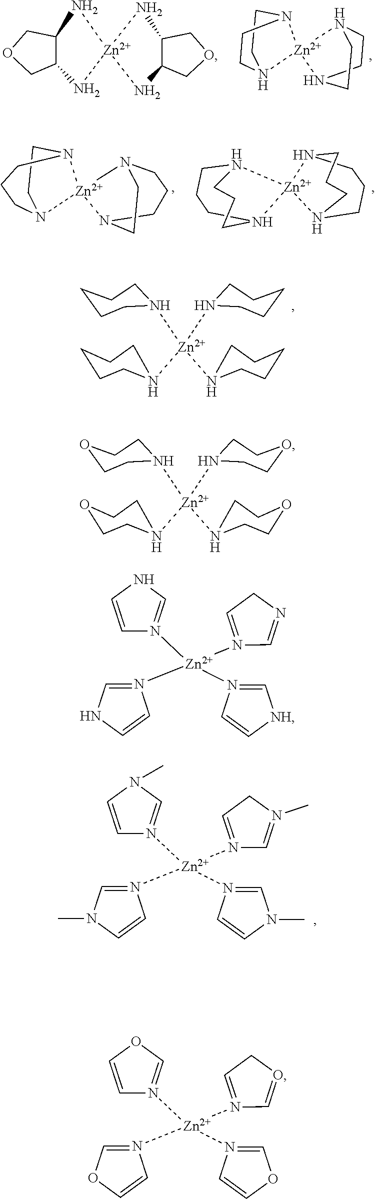 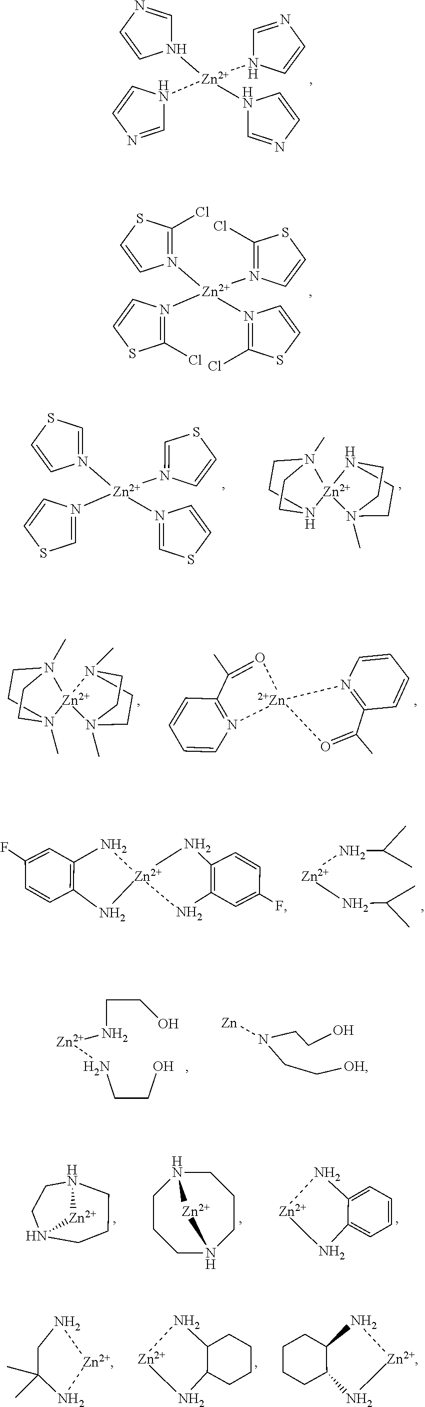 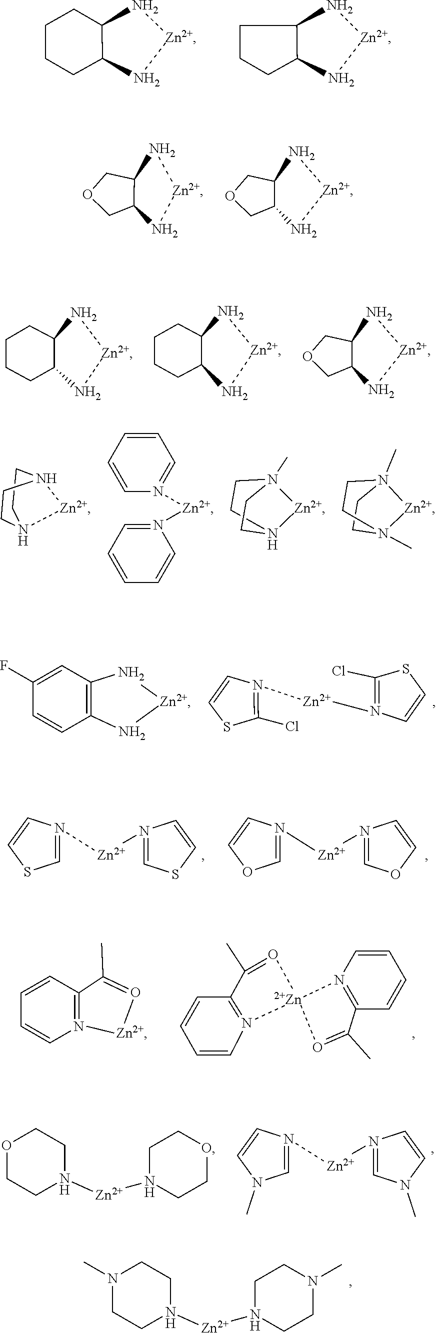 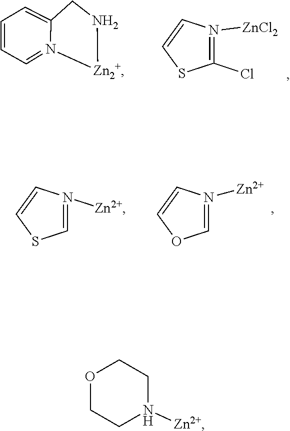
all the complex cations above can be formed as a salt with an anion which is selected from, Cl⁻, Br⁻, I⁻, SO₄²⁻, HSO₄⁻, NO₃⁻, PO₄³⁻, HPO₄²⁻, H₂PO₄⁻, CO₃²⁻, HCO₃⁻, HCOO⁻, CH₃COO⁻, F₃CCOO⁻, Cl₃CCOO⁻, FCH₂COO⁻, ClCH₂COO⁻, F₂CHCOO⁻, Cl₂CHCOO⁻, BF₄⁻, SO₃²⁻, HSO₃₋, CH₃SO³⁻, C₆H₅CH₂SO³⁻, C₆H₅SO³⁻, C₆H₅COO⁻, C₆H₅CH₂COO⁻, C₆F₅O⁻, C₆H₄(OH)COO⁻, C₆H₂F₃O⁻, C₆H₄(NO₂)O⁻, C₆H₂(NO₂)₃O⁻, the transition metal cation-amino complex in the reaction solution are 0.01 mM-1.0 mM in concentration, or 0.5˜20 equivalents in moles of the antibody, and can be added to the reaction solution with a water-miscible organic solvent, selected from ethanol, methanol, propanol, propandiol, DMA, DMF, DMSO, THF, or CH₃CN;
wherein the said reductant is tris(2-carboxyethyl)phosphine (TCEP) (P(CH₂CH₂COOH)₃), or tris(hydroxypropyl)phosphine (P(CH₂CH₂CH₂OH)₃), and is used at 1.0-20 equivalents in moles of the antibody in the reaction;
wherein the oxidant to be added in step(c) is selected from DHAA, Fe³⁺, I₂, Cu²⁺, Mn³⁺, MnO₂, or mixture of Fe³⁺/I⁻, the oxidant used in the reaction solution is 0.02 mM-1.0 mM in concentration, or 1-100 equivalents in moles of the antibody;
wherein pH in the conjugation reaction is between about 5.0 to 8.0; and up to 30% of a water mixable (miscible) organic solvent, selected from DMA, DMF, ethanol, methanol, acetone, acetonitrile, THF, isopropanol, dioxane, propylene glycol, or ethylene diol can be added as a co-solvent in water based buffer solution;
wherein temperature in the conjugation reaction is between about −5° C. to 40° C., time of the process of conjugation reaction is between about 15 min to about 48 hours;
wherein the resulted conjugate can be purified by gel filtration on a Sephadex G25 or Sephacryl S300 column, adsorption chromatography, ion (cation or anion) exchange chromatography or by dialysis (ultrafiltration or hyperfiltration (UF) and/or diafiltration (DF).

50. The method of claim 49, wherein the resulted conjugate of formula (I), (II), (III), (X), (XI) or (XII) are mainly (over 60%) linked to the cysteine sites between heavy-light chains of the antibody, and when drug/antibody ratio (DAR) is set to be 4, the distributions in percentage of the numbers of drugs in the antibody are: D0<5%, D2<10%, D4>60%, D6<10%, D8<10%.
wherein drug/antibody ratio (DAR) of the conjugates is measured by UV at wavelength of range 240-380 nm, by hydrophobic interaction chromatography (HIC-HPLC), reverse phase chromatography (RP-HPLC), Capilaryelectrophoresis (CE), LC-MS, CE-MS, or LC-MS/MS.

51. The antibody-drug conjugate of claim 40 having one of the following structures of: 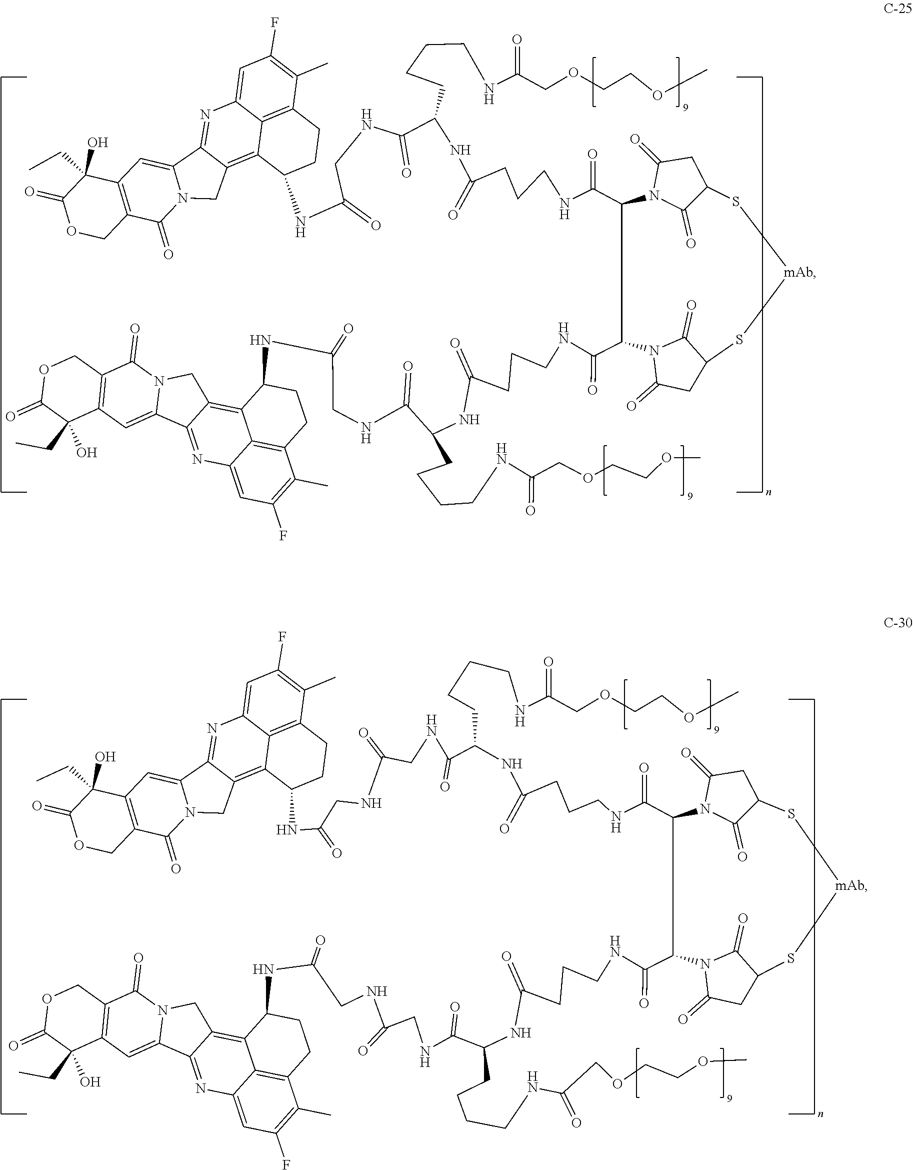 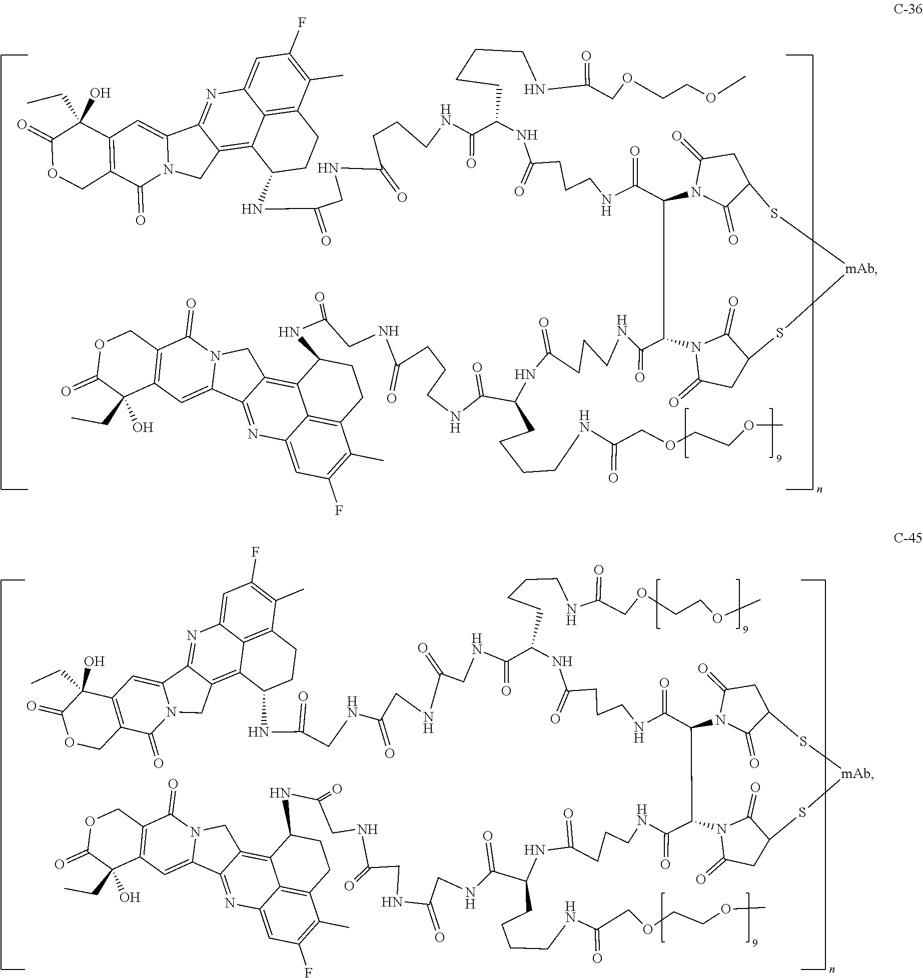 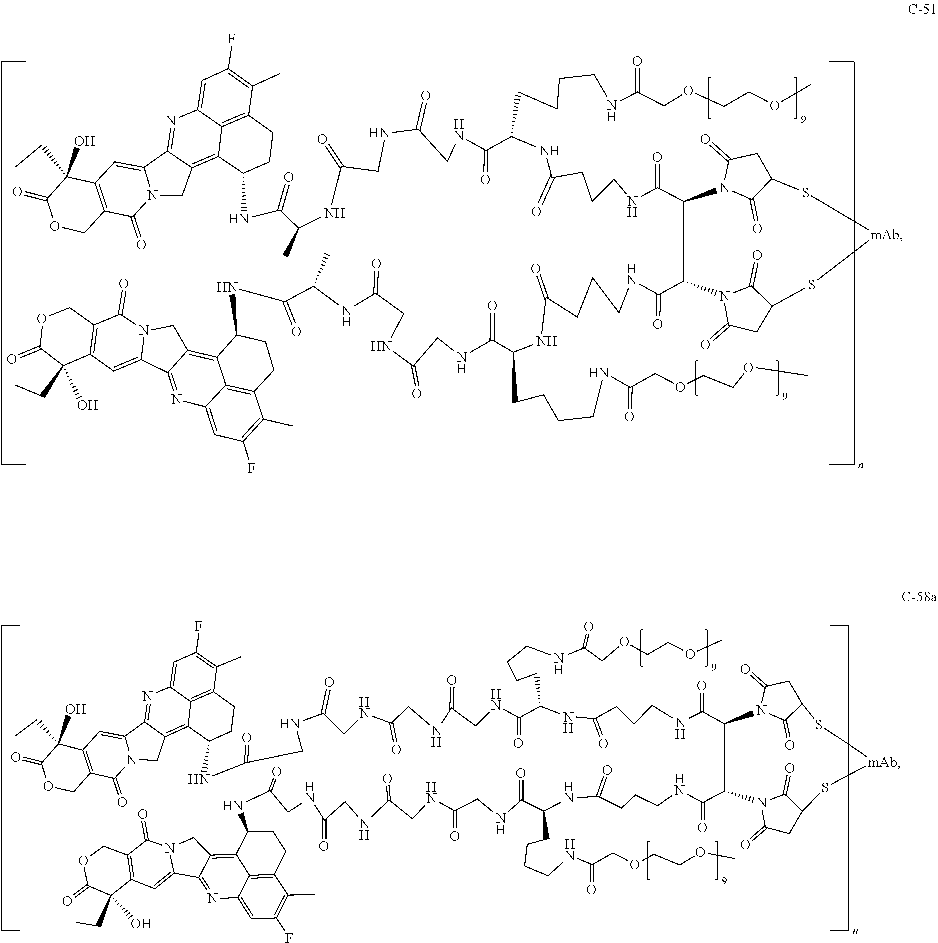 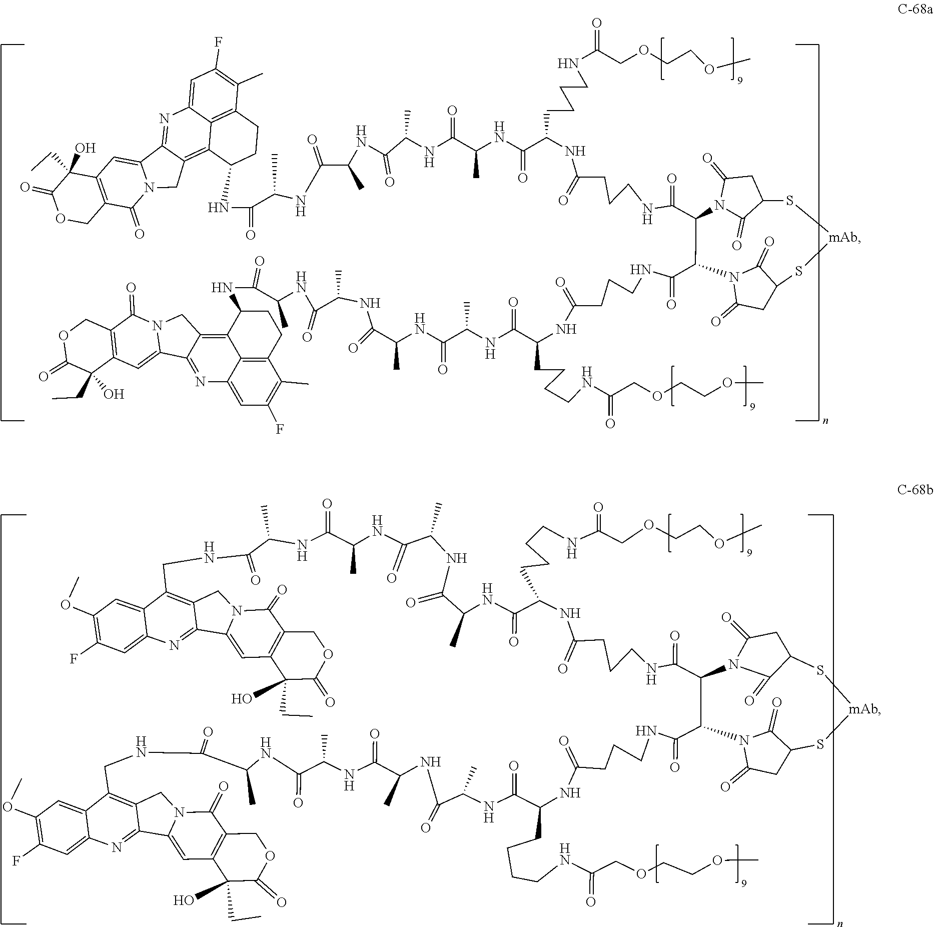 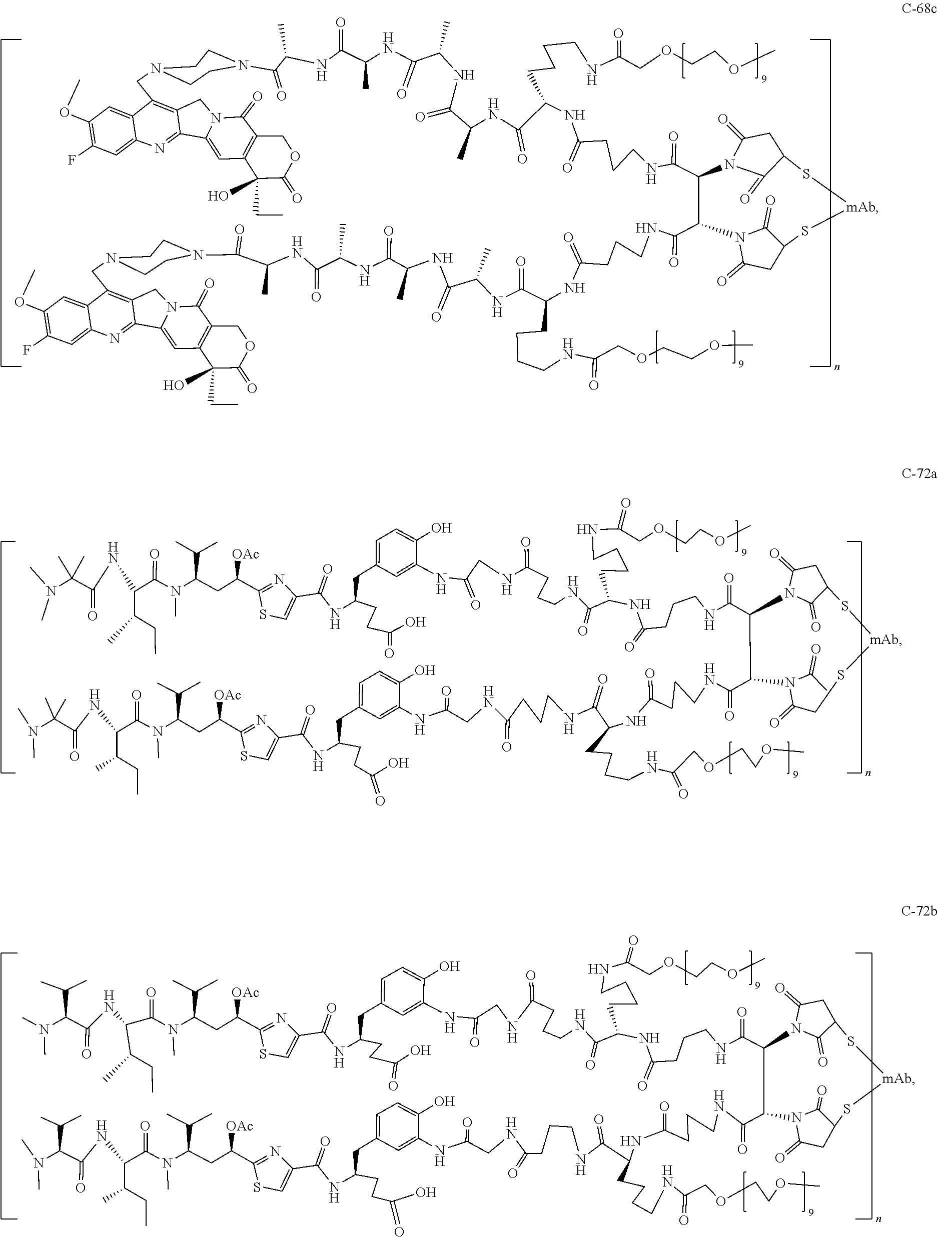 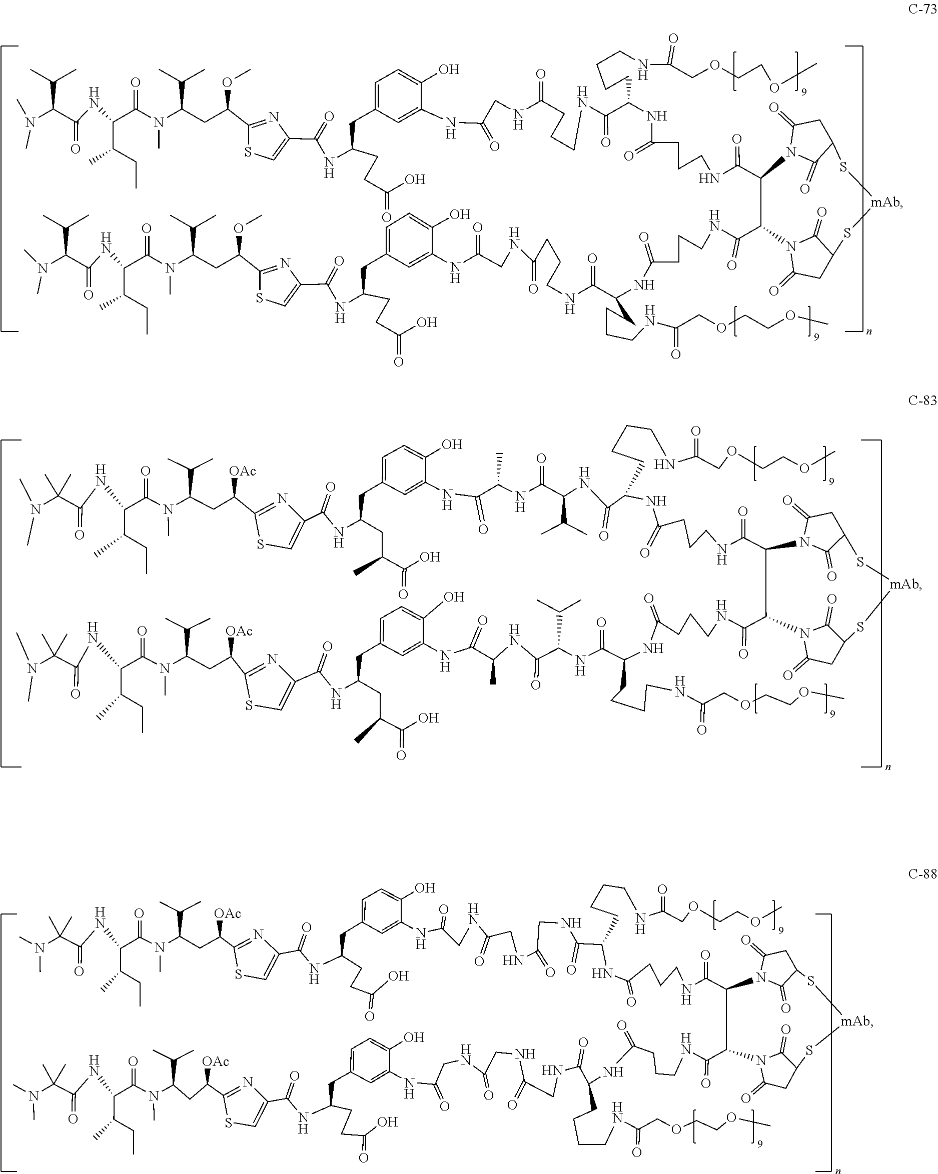 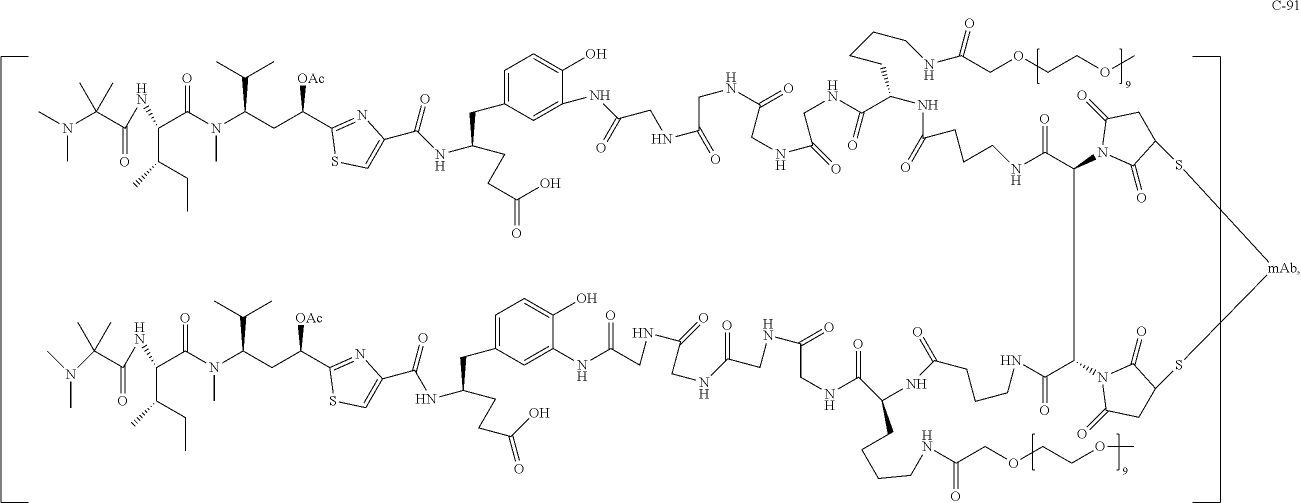 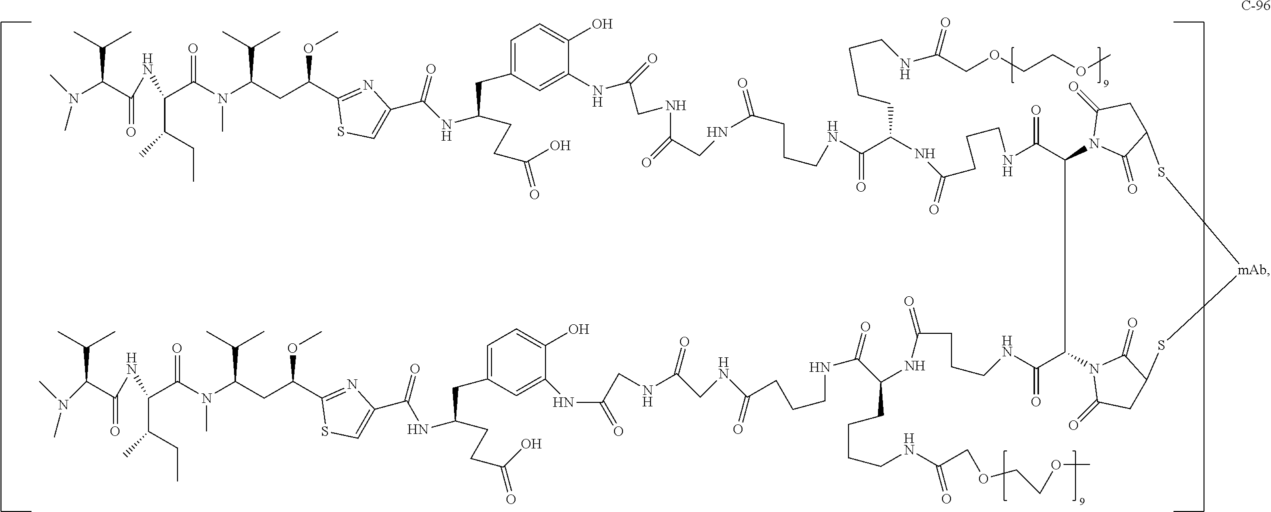 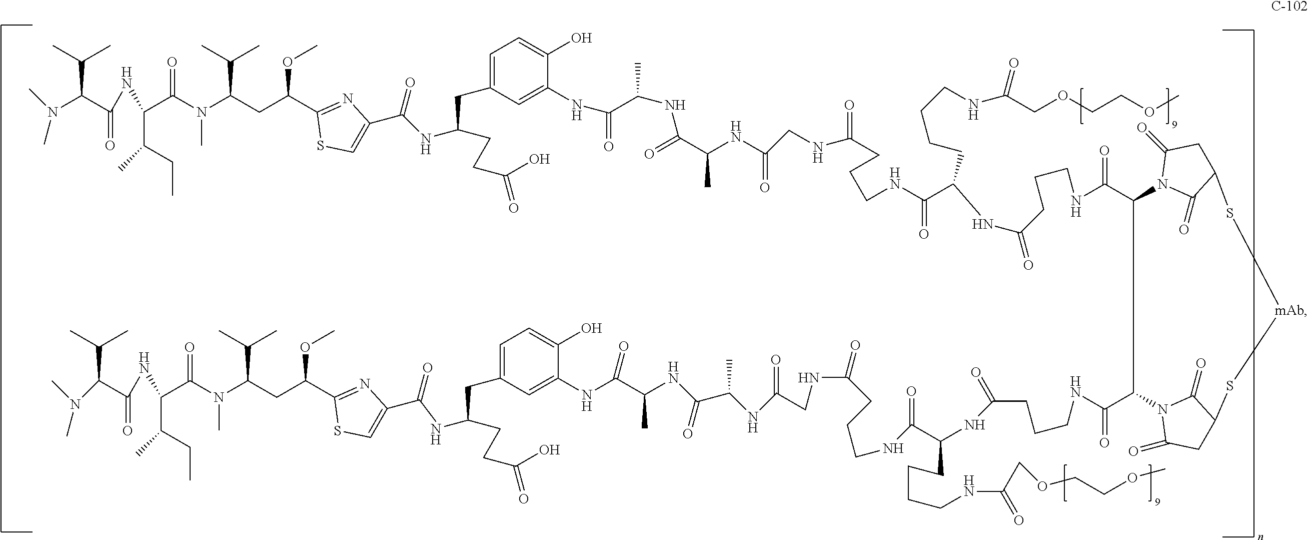 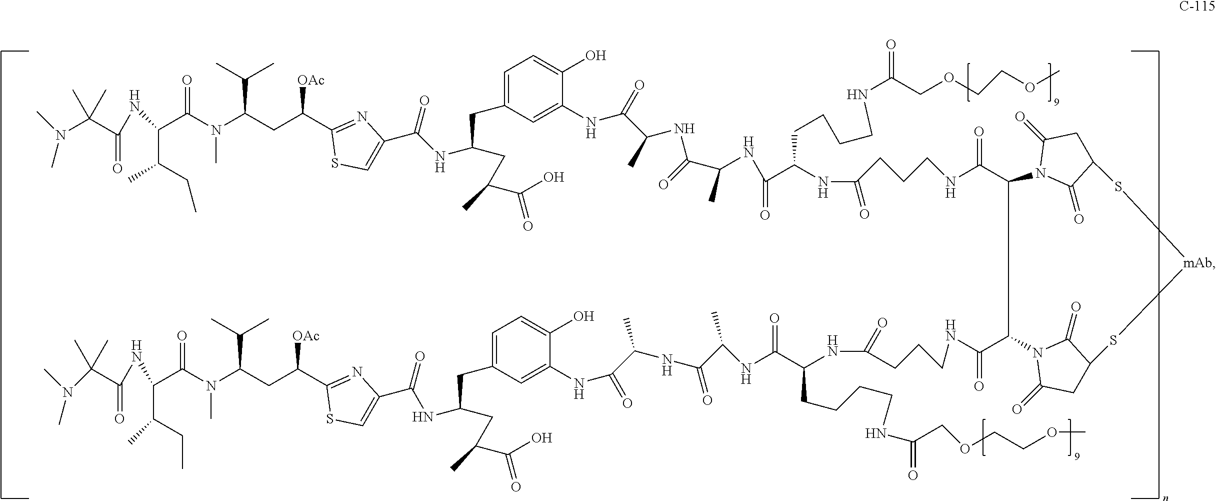 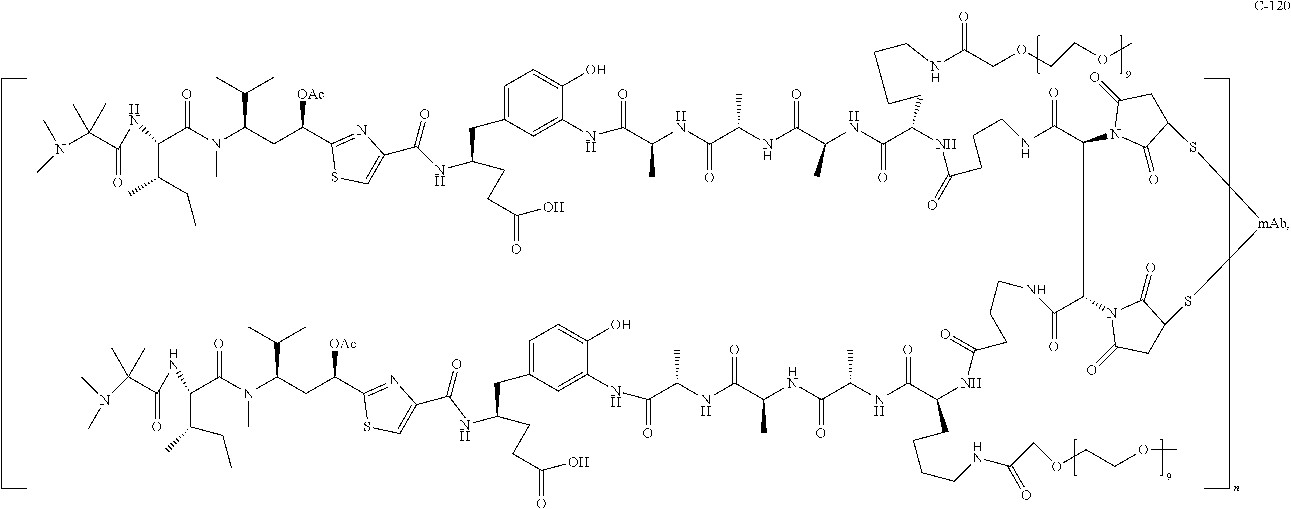 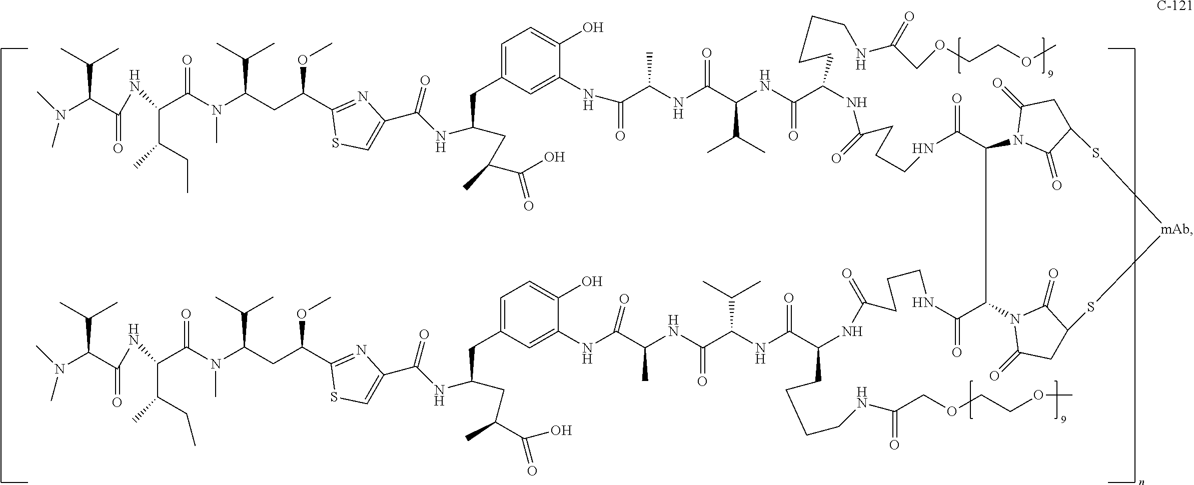 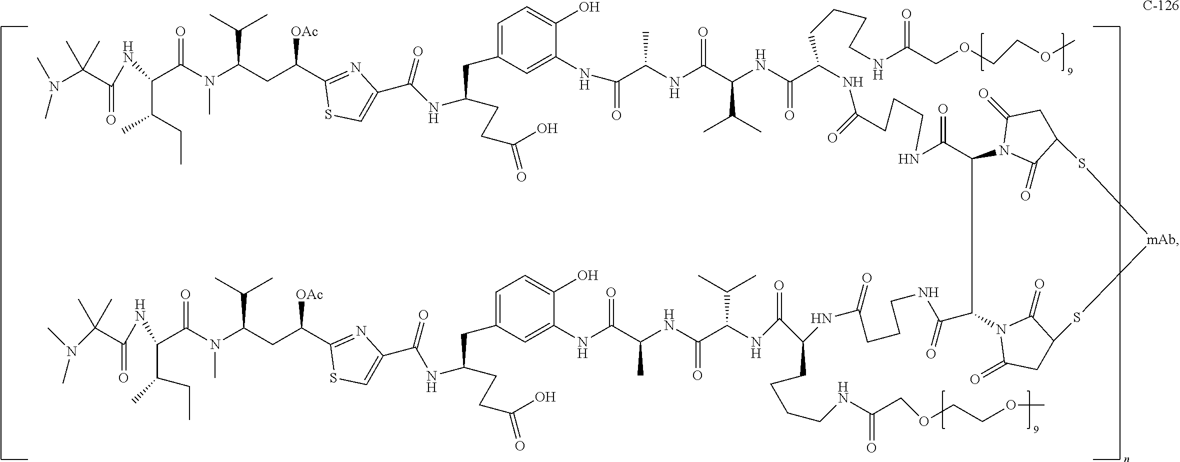 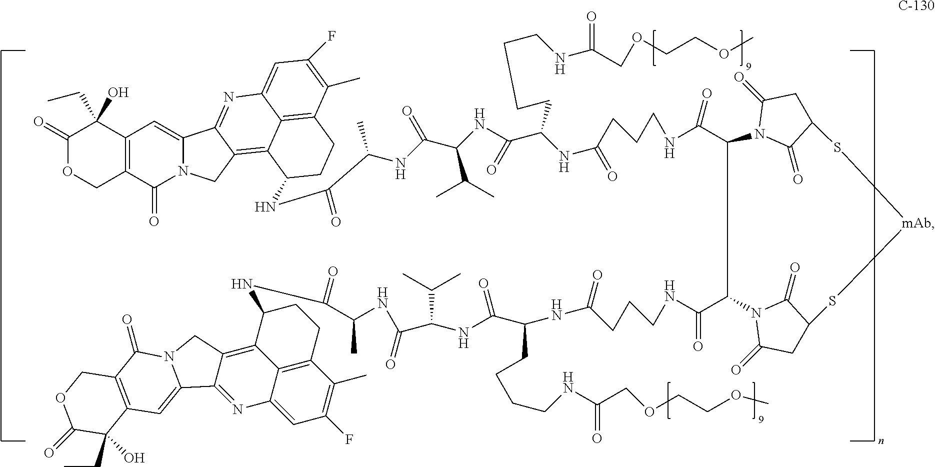 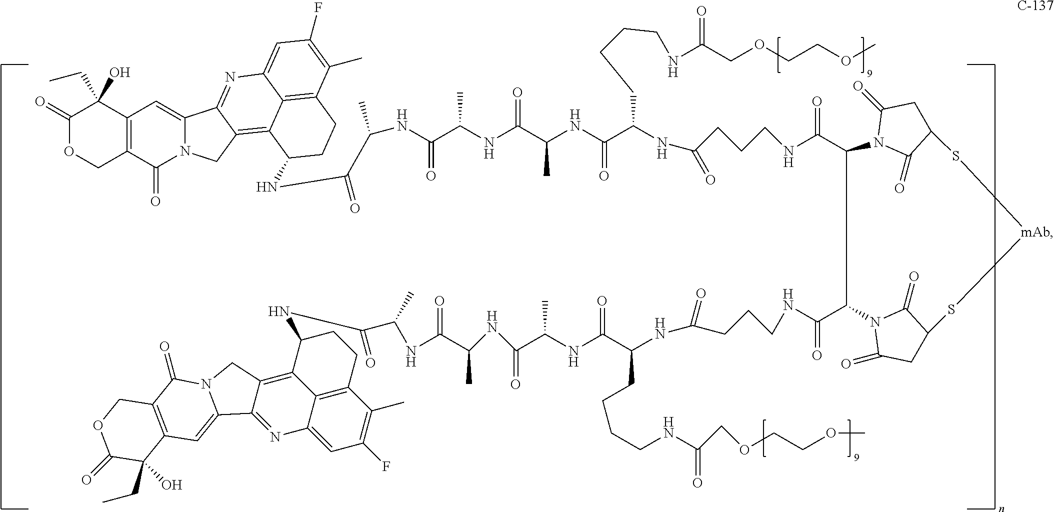 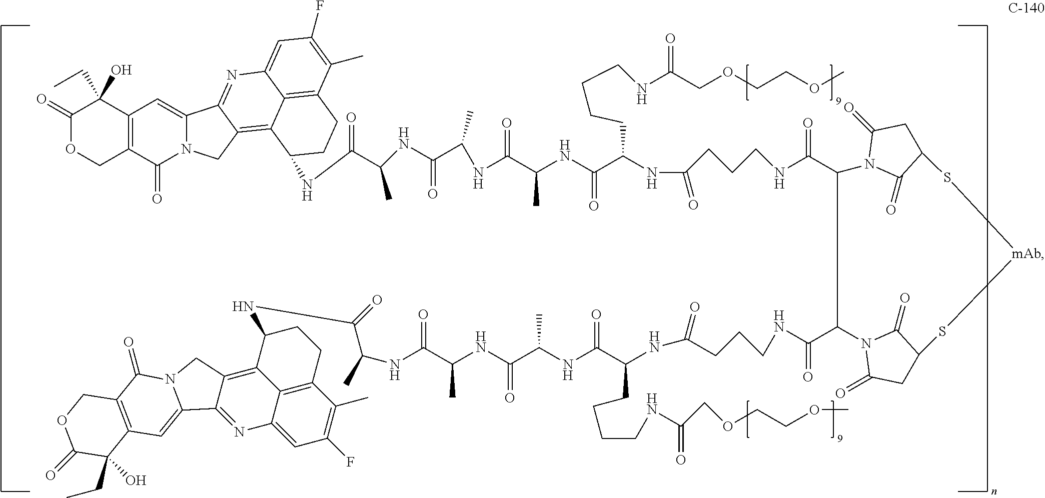 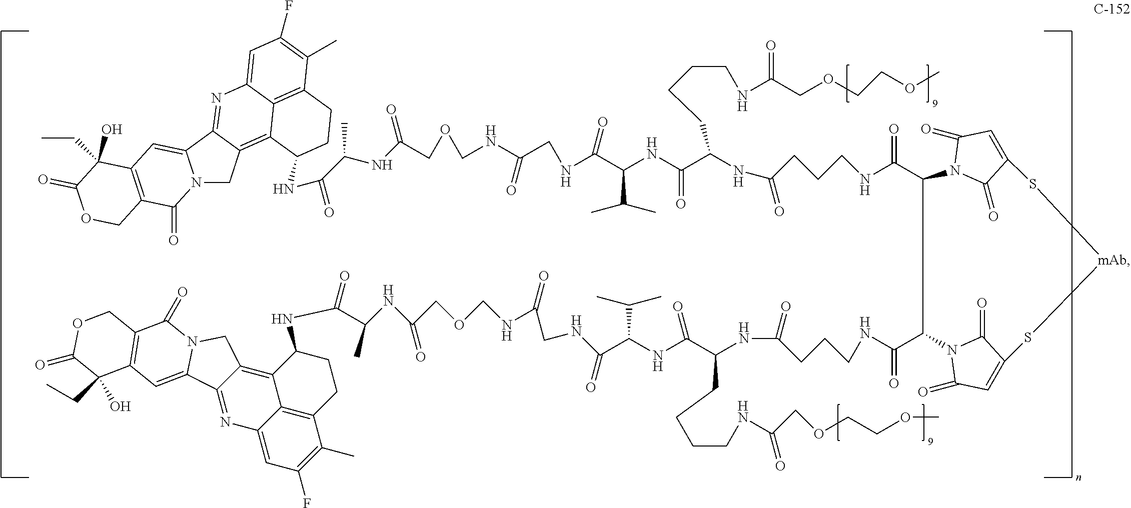 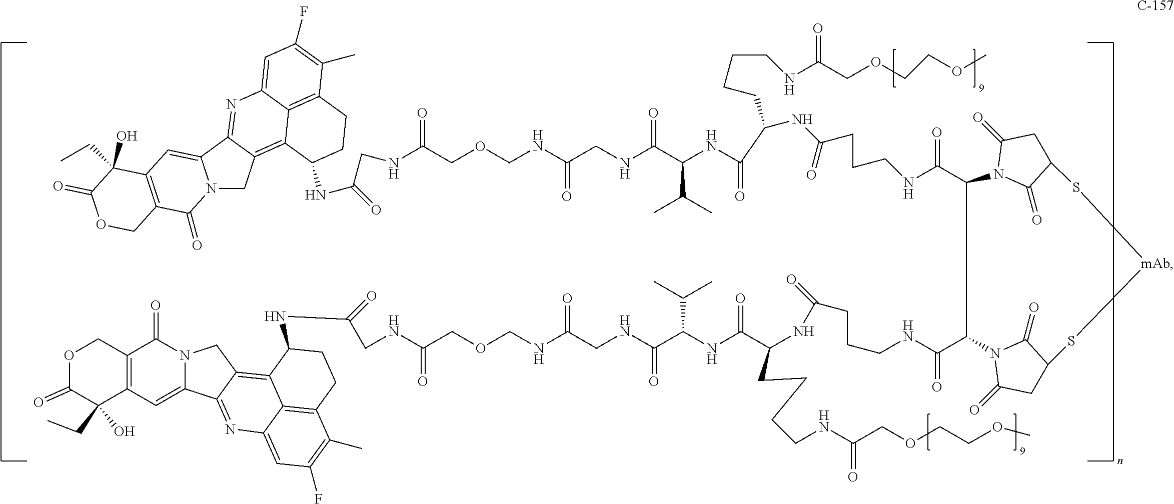 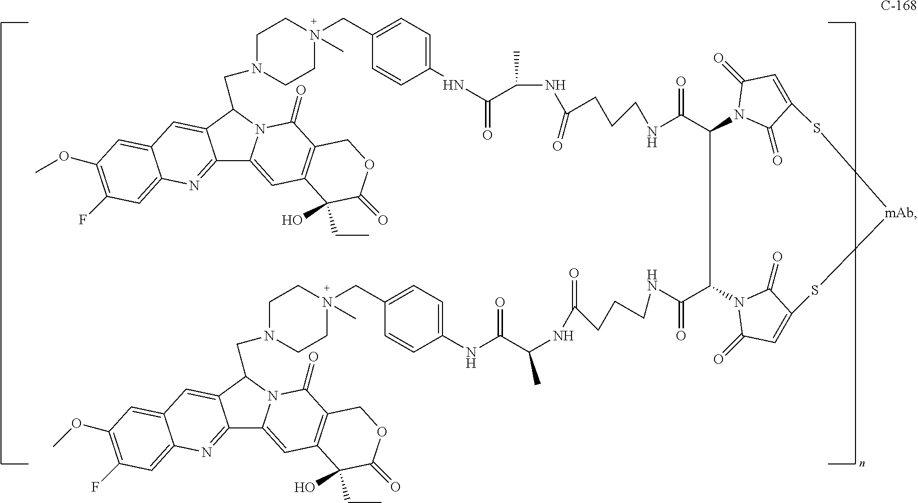 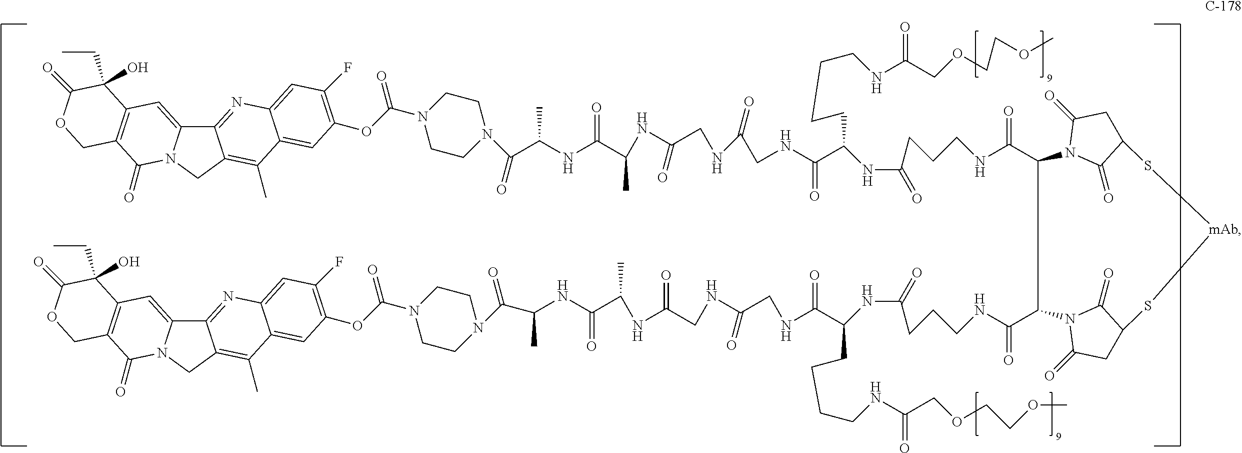 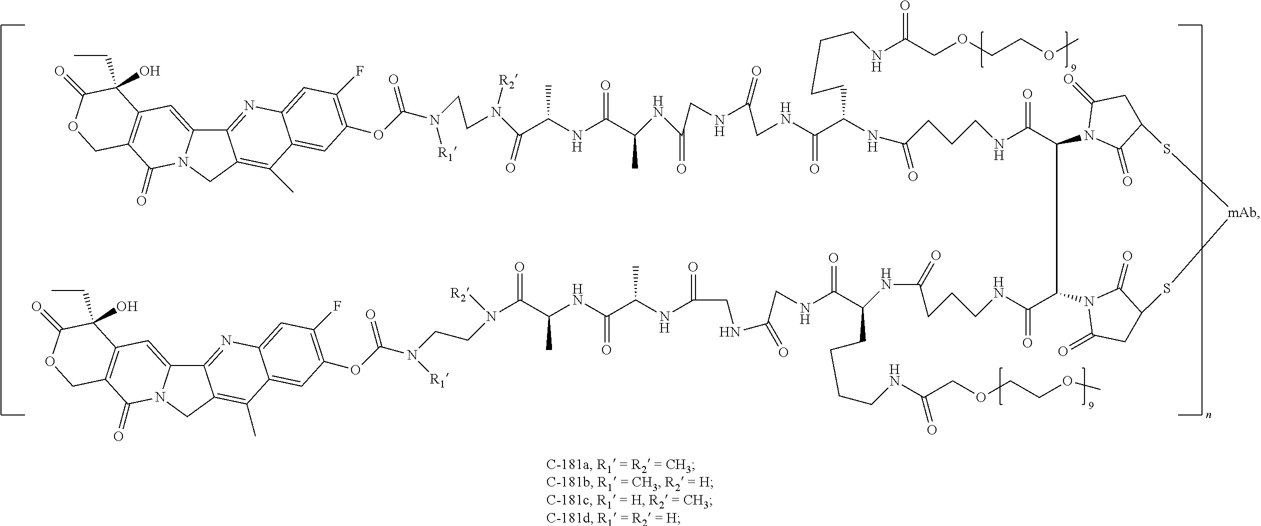 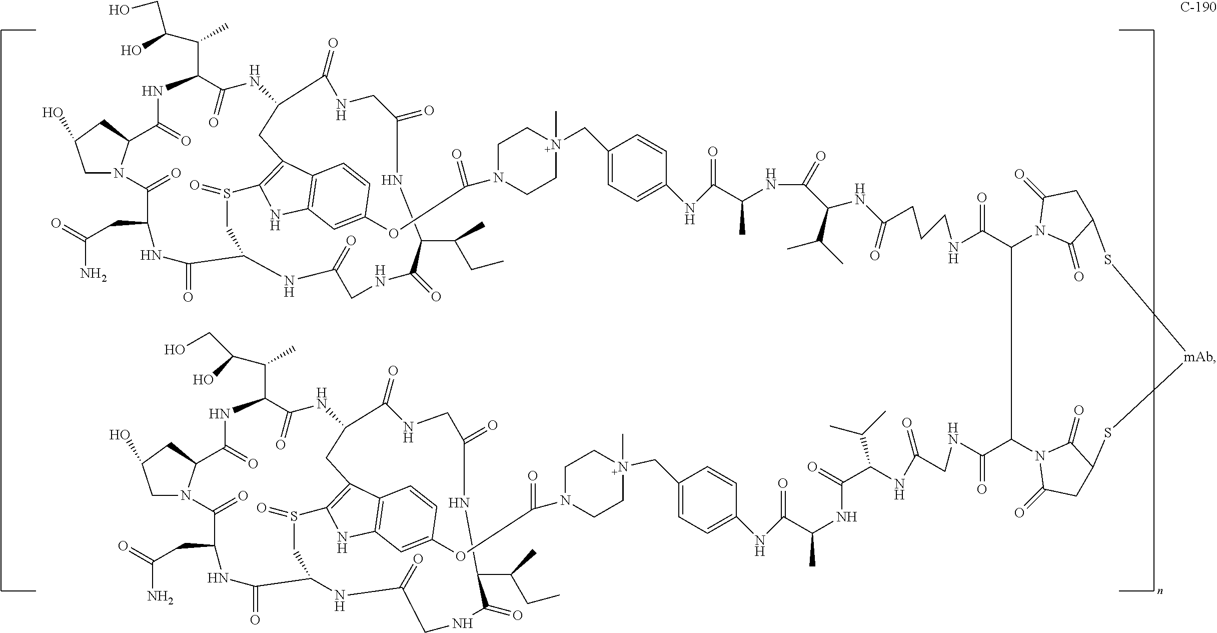 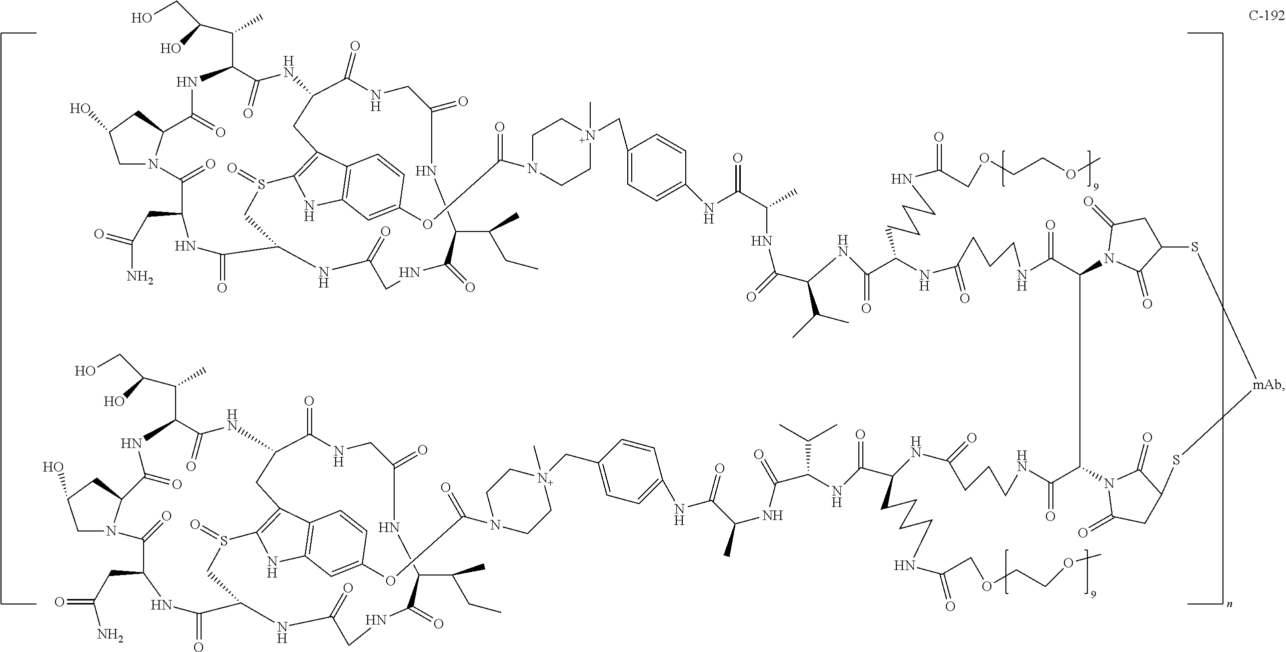 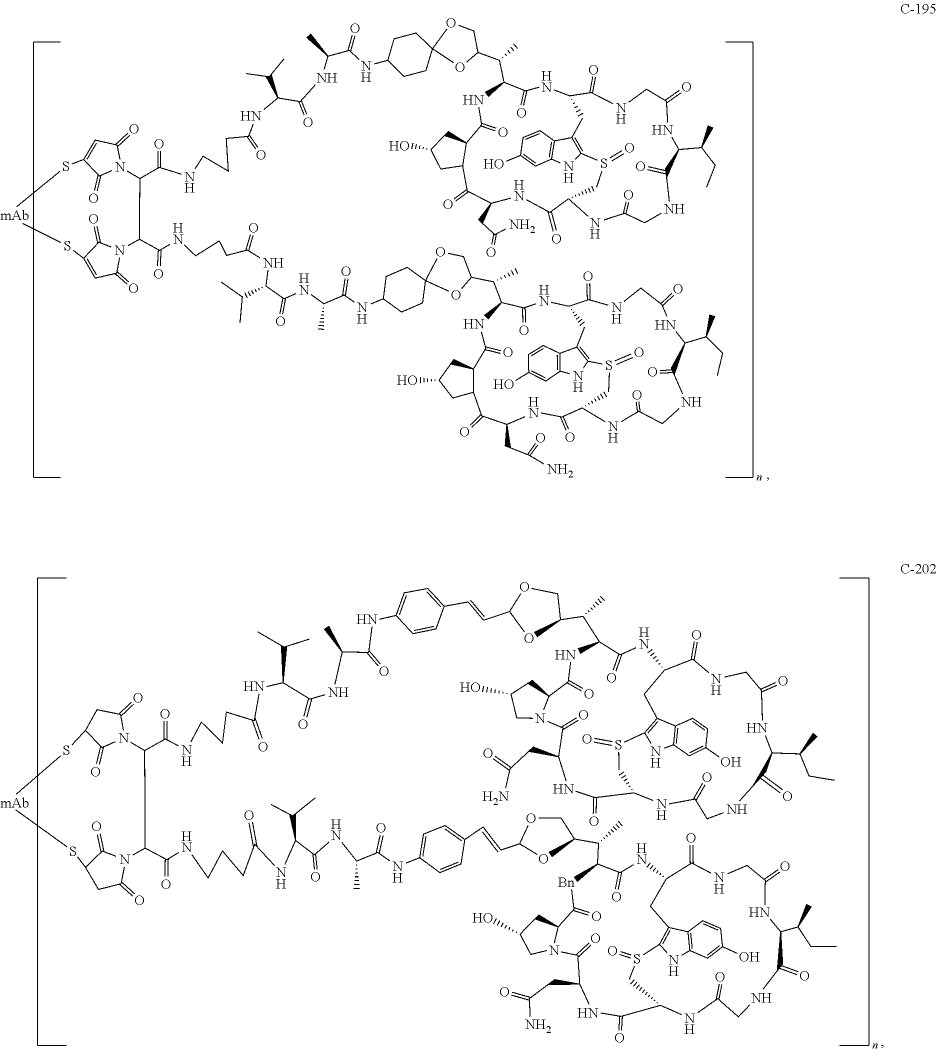 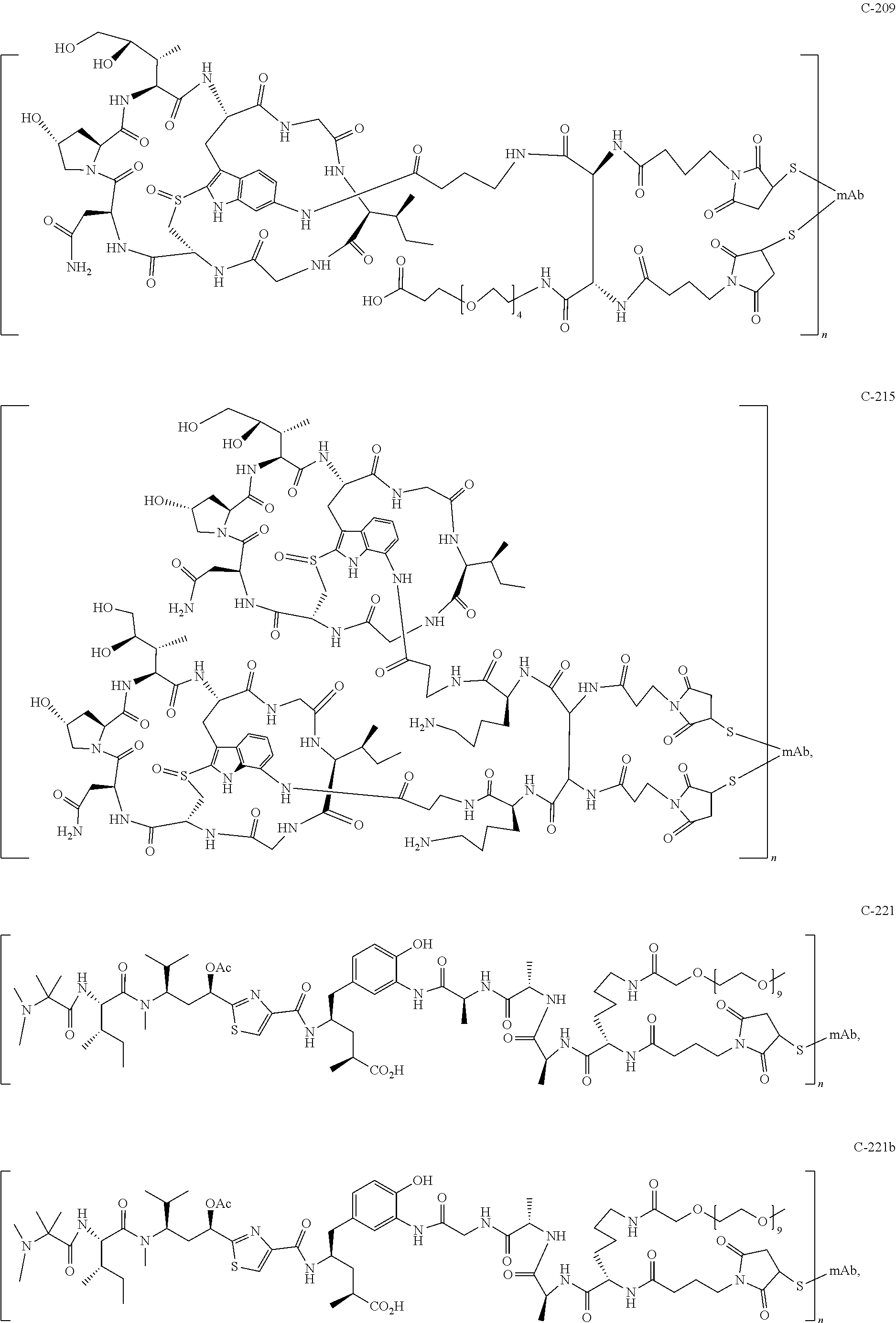 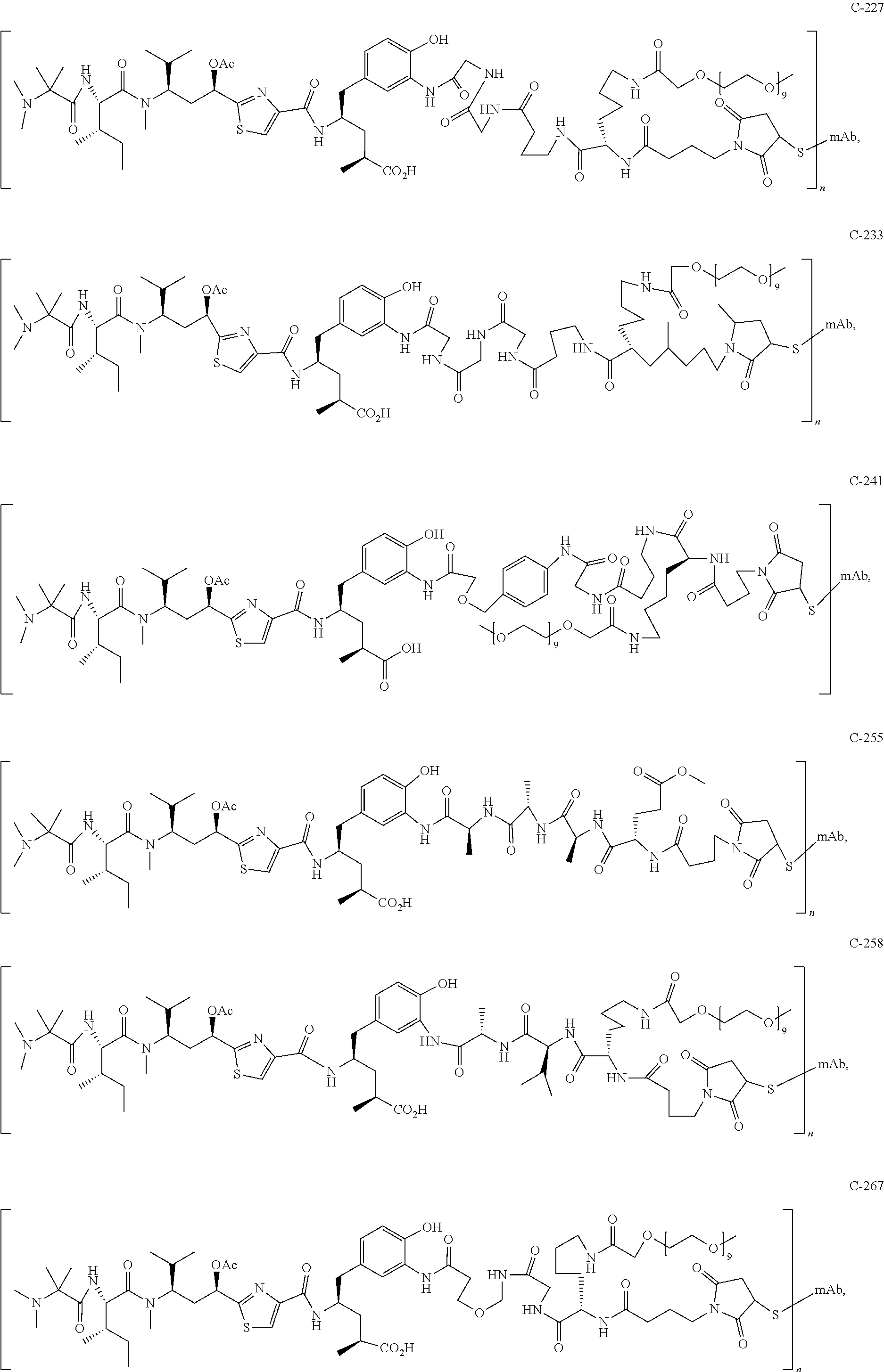 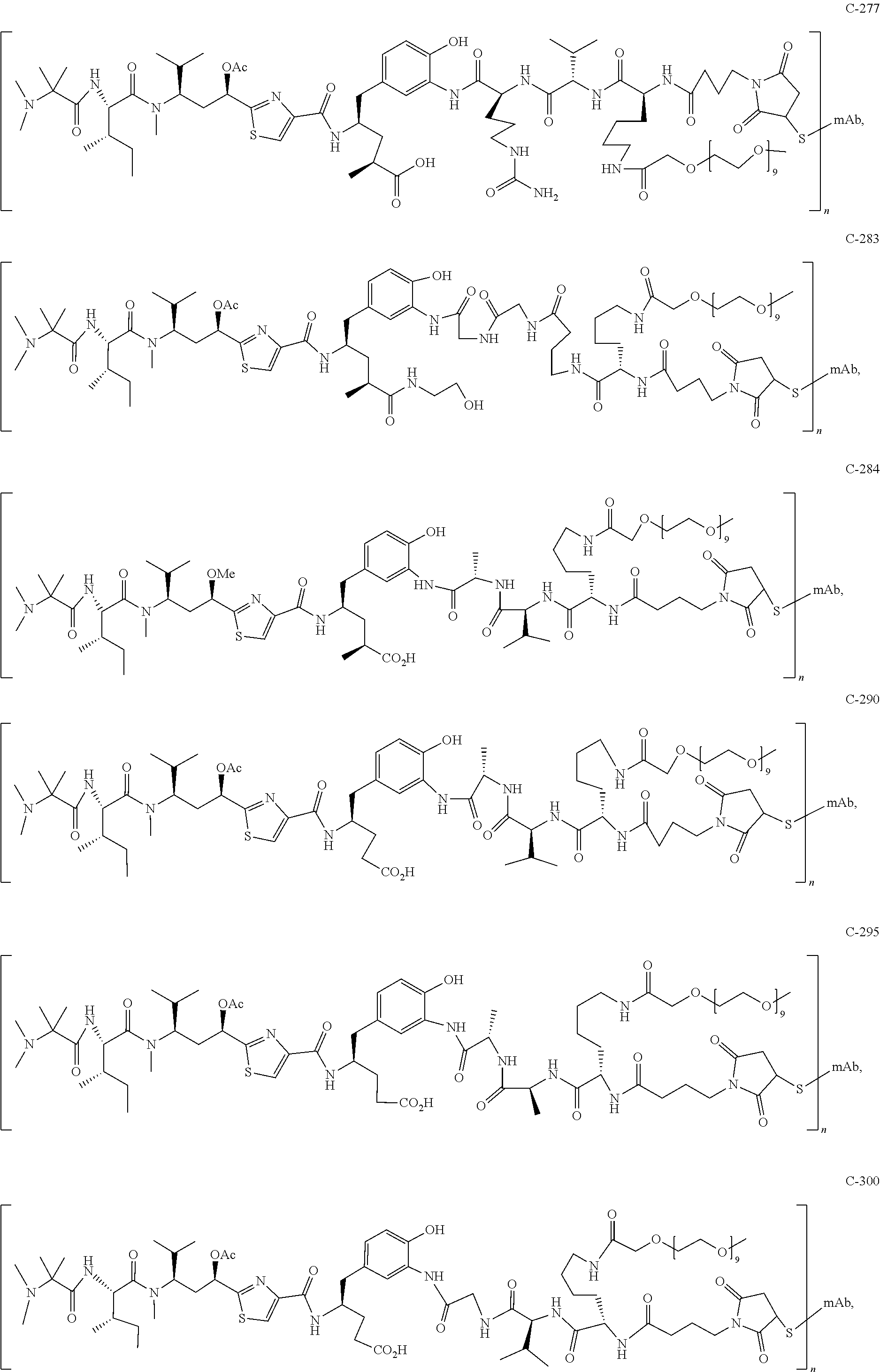 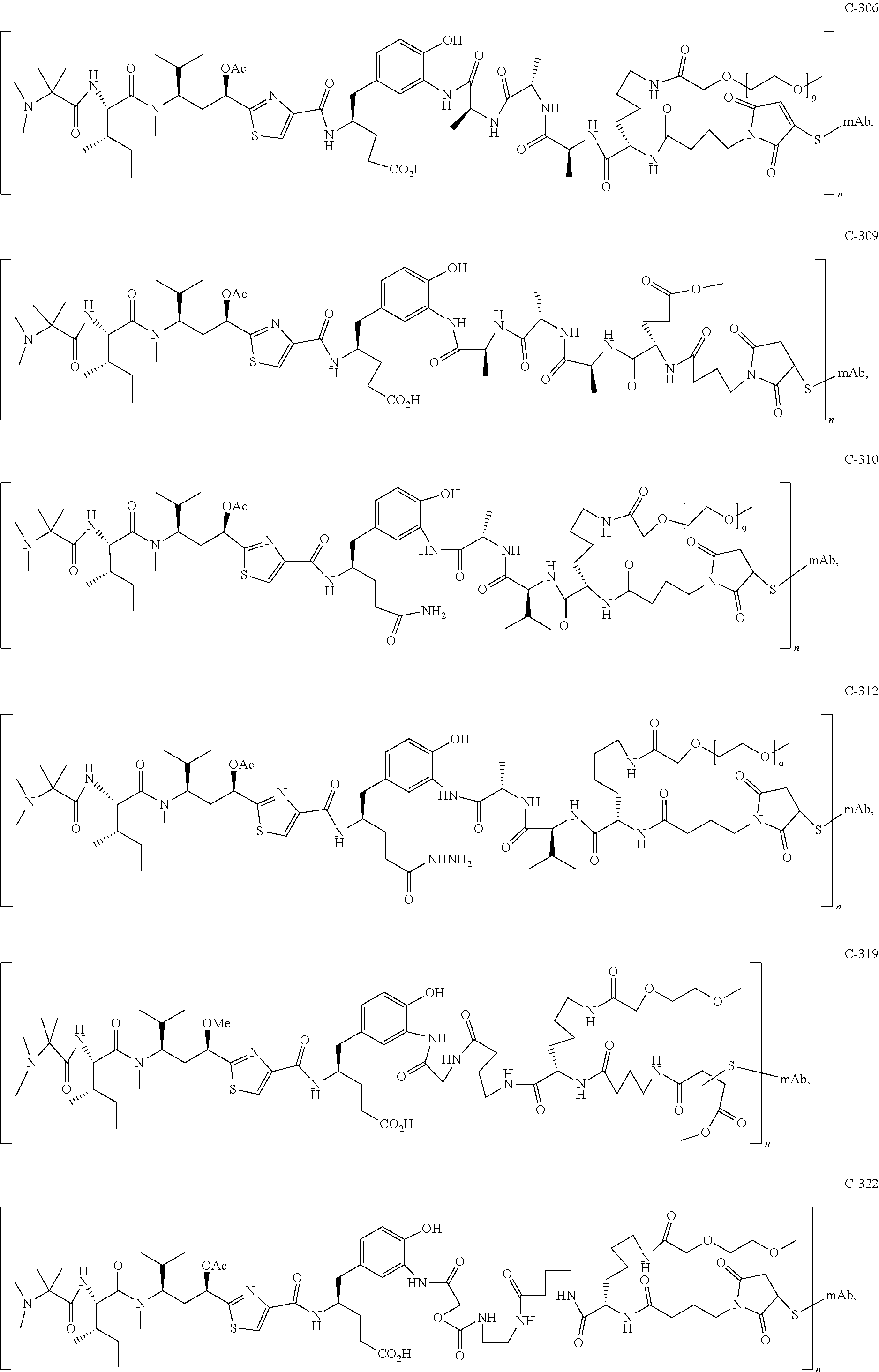 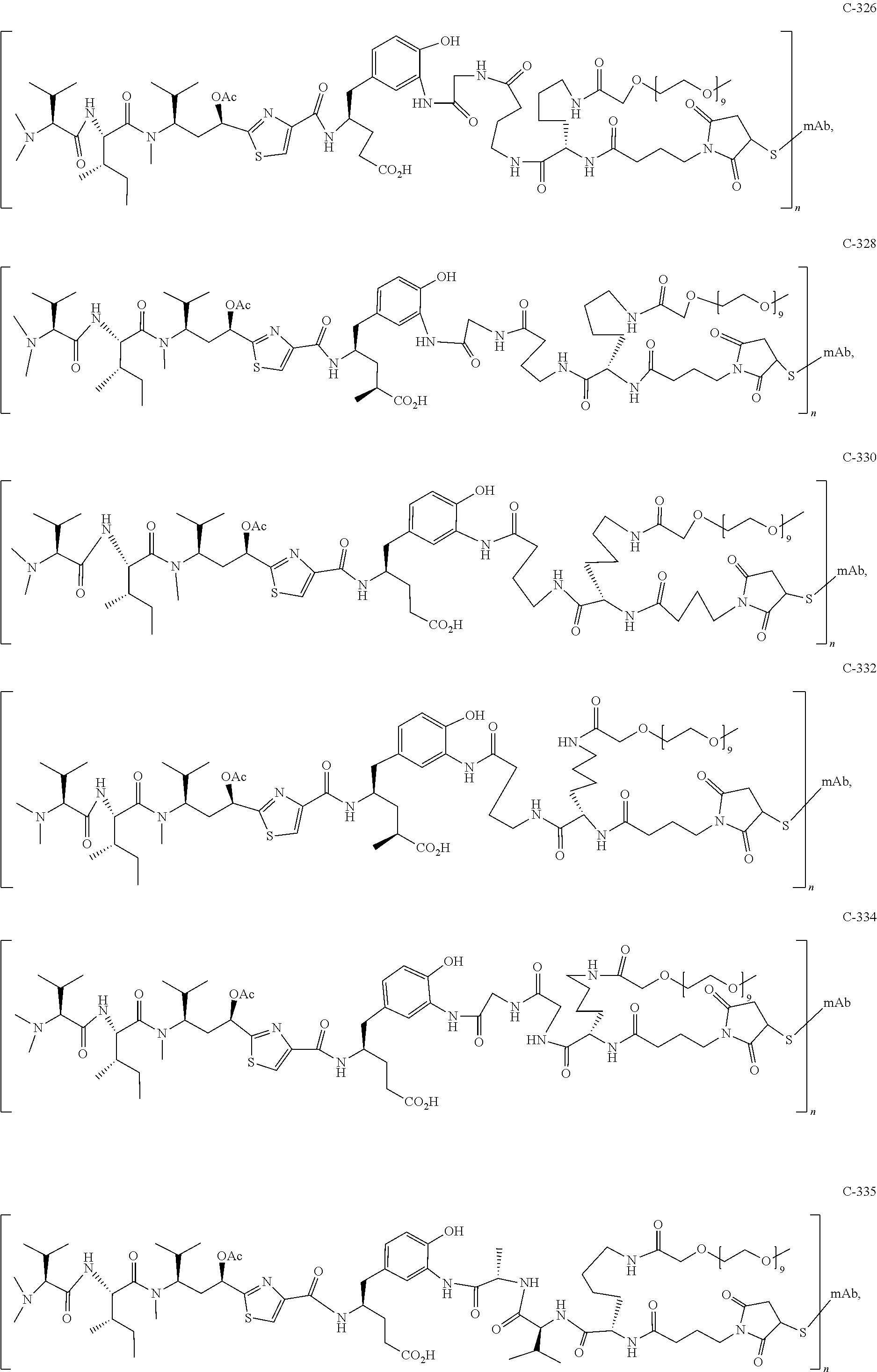 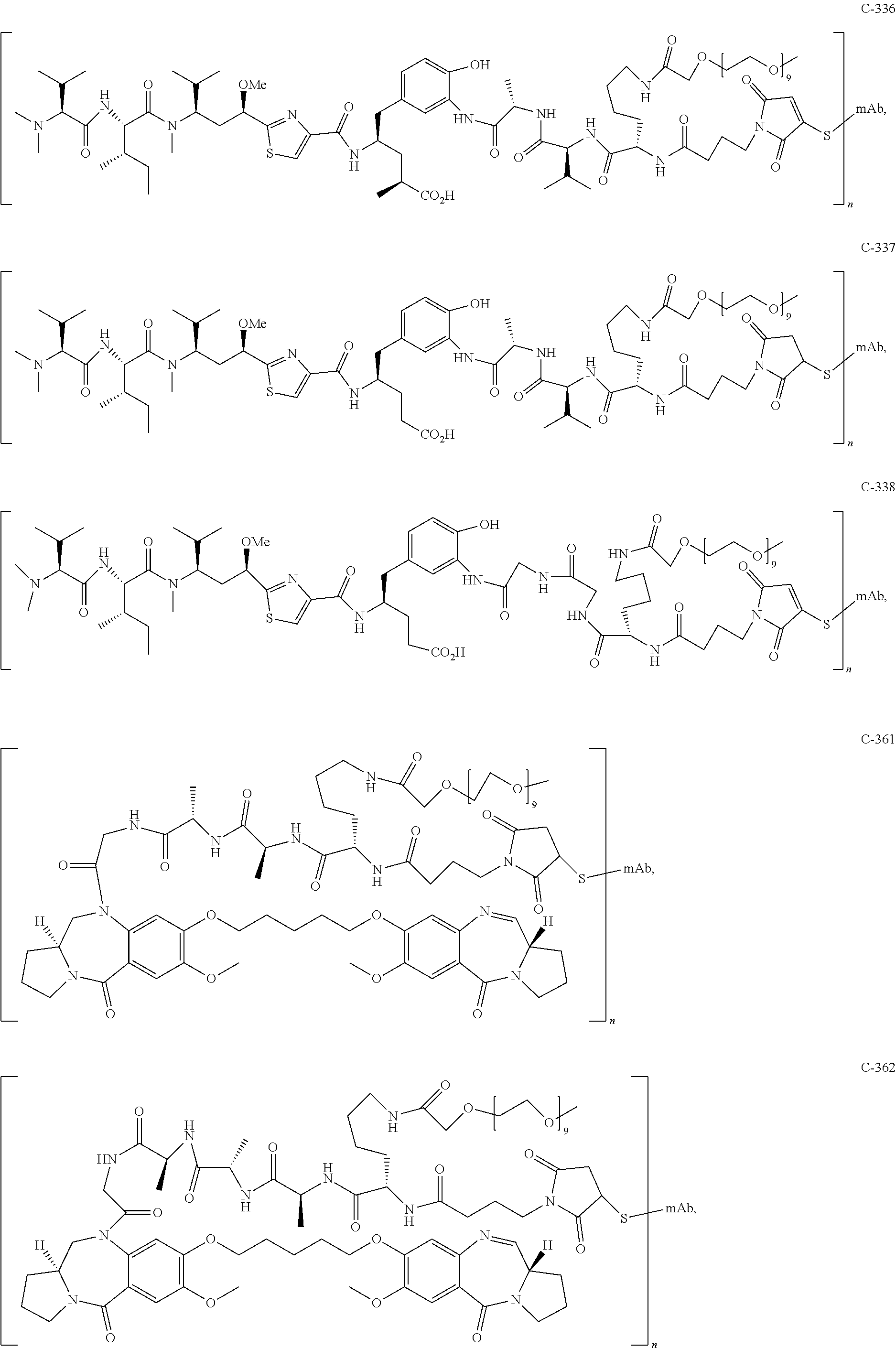 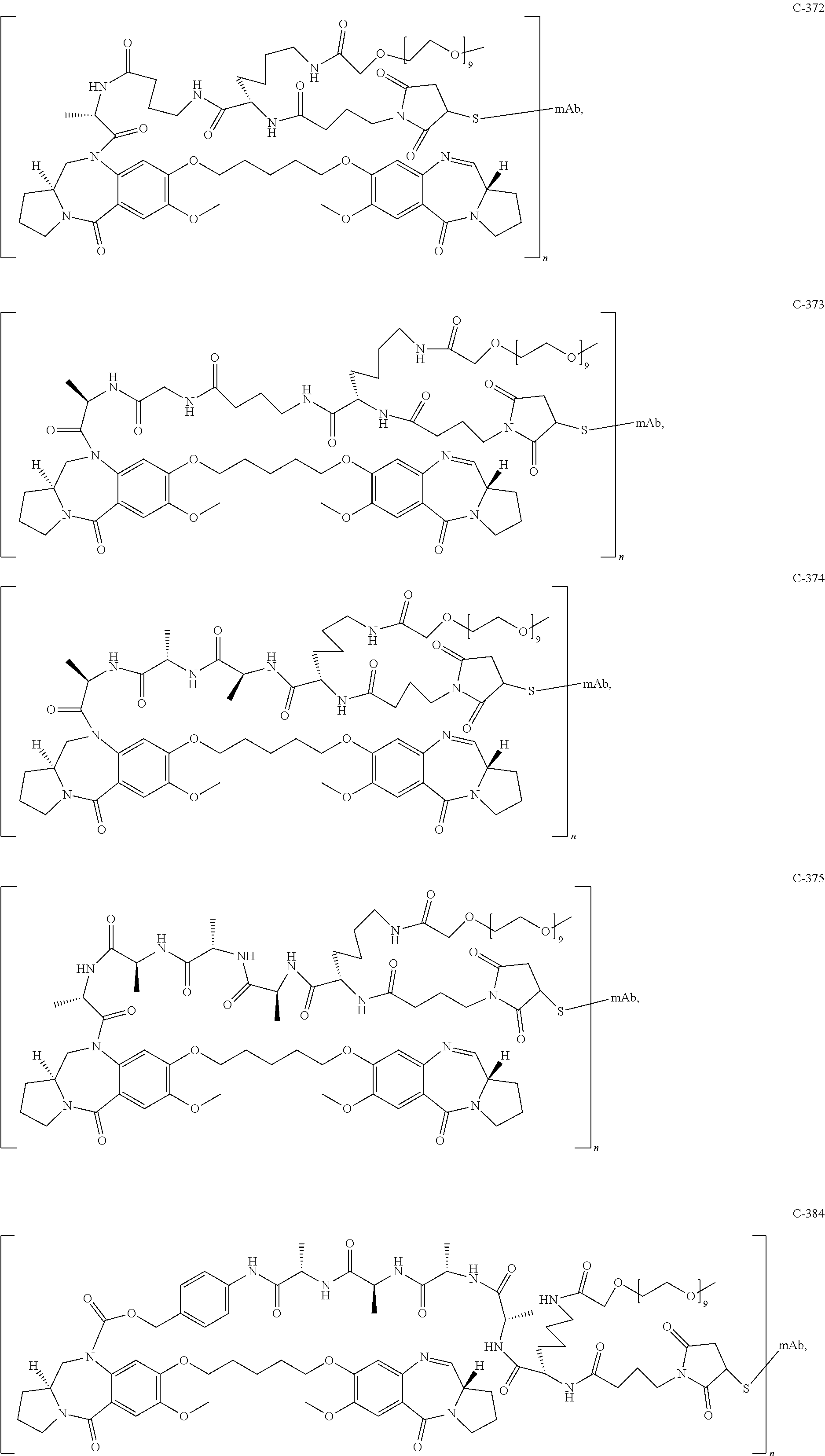 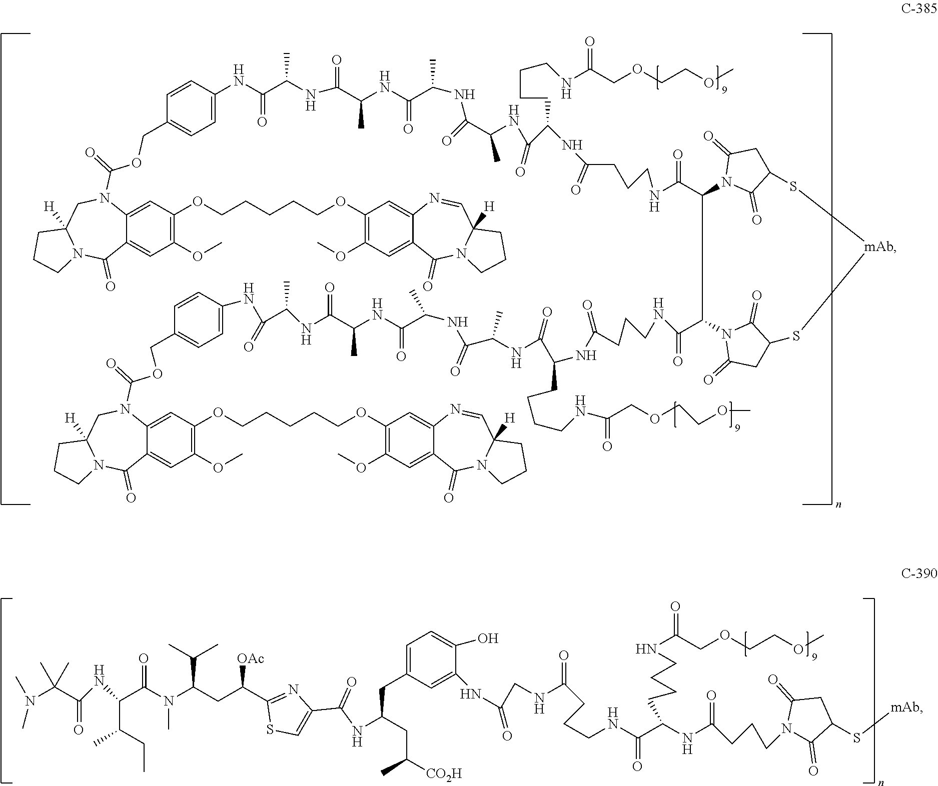 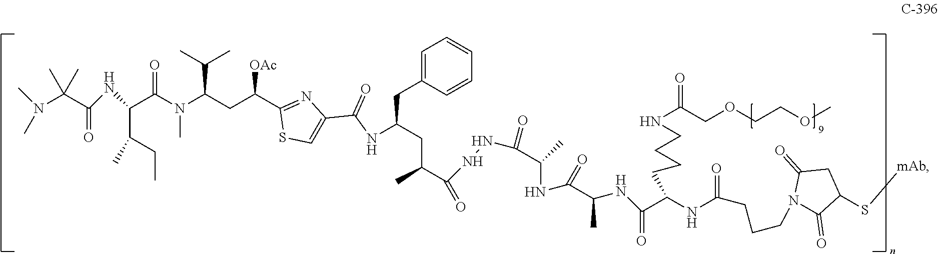 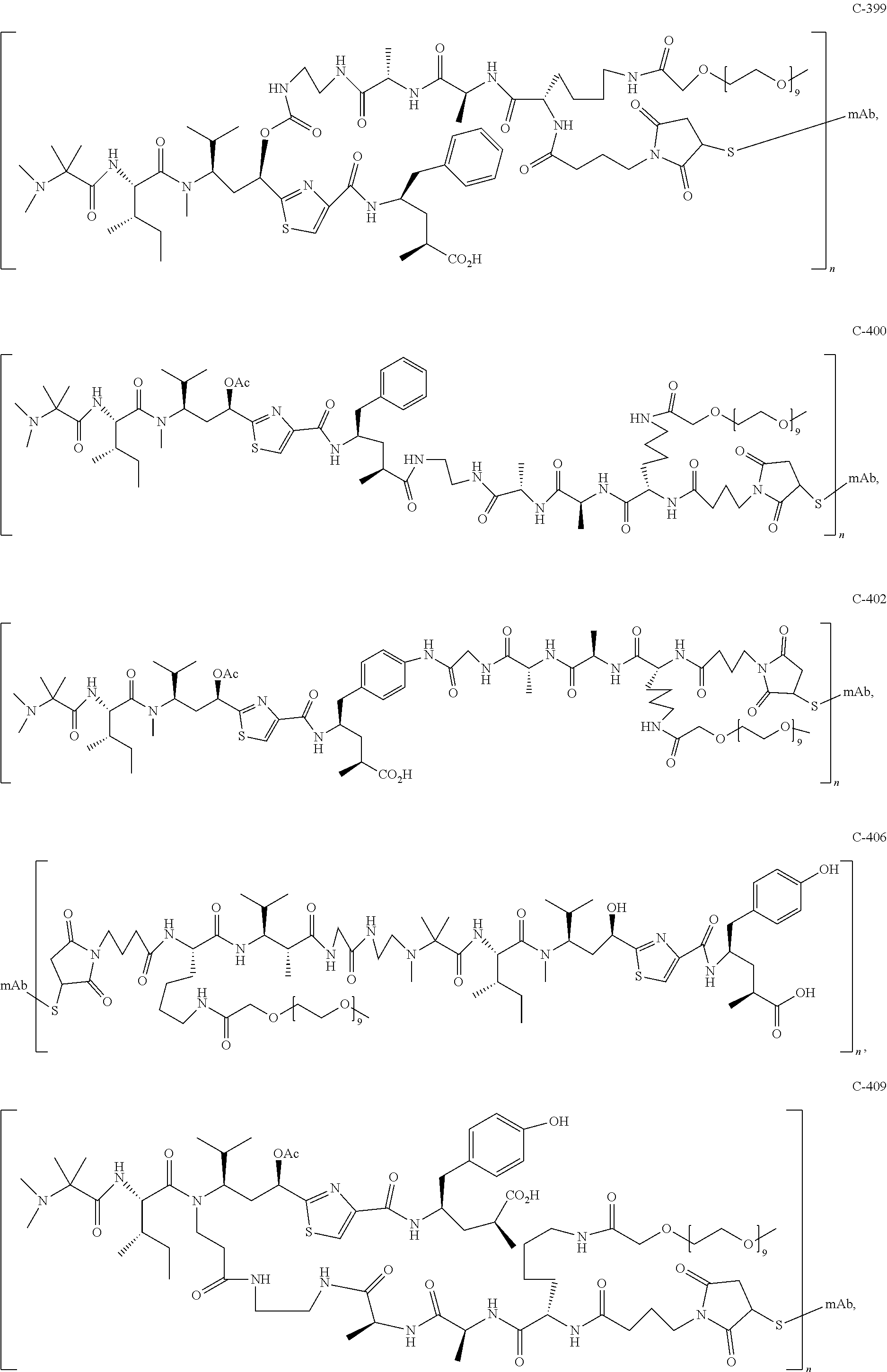 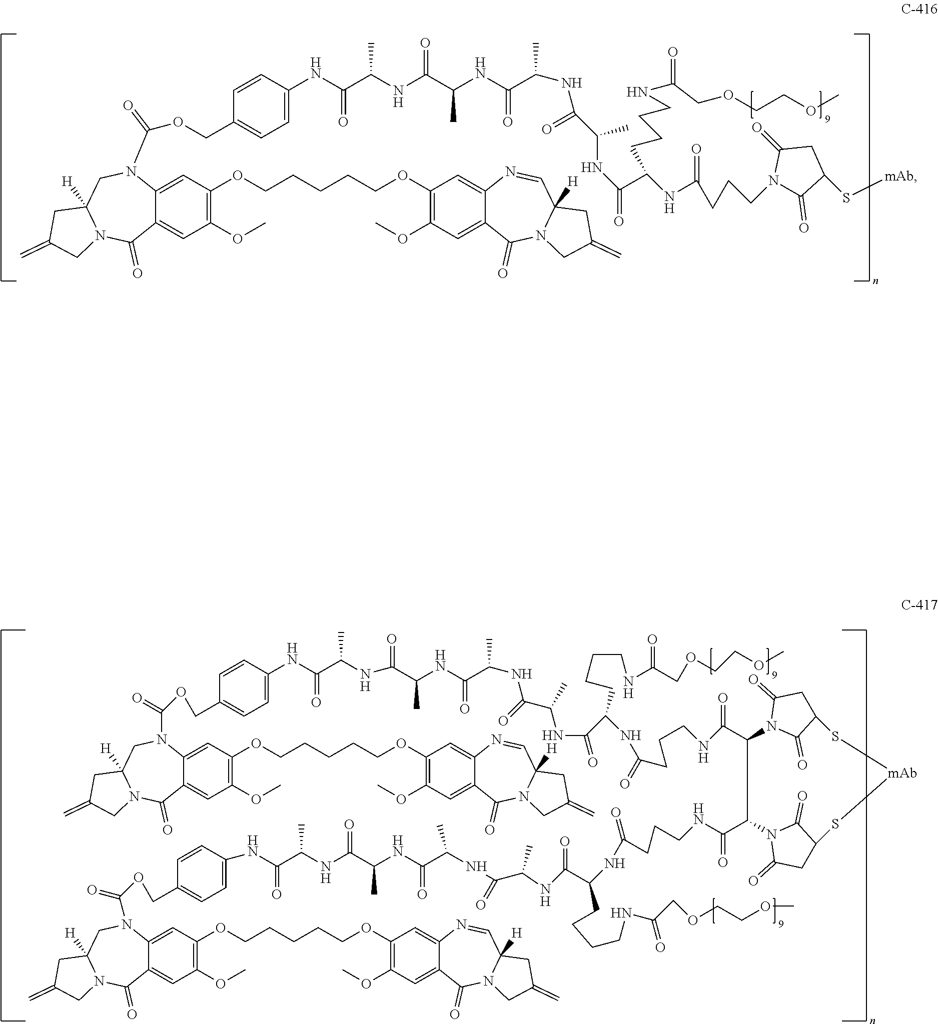
wherein mAb is the antibody, n=1-20.

52. The drug/linker complex of formula (IV), (V) and (VI) of claim 46 have the following structures of

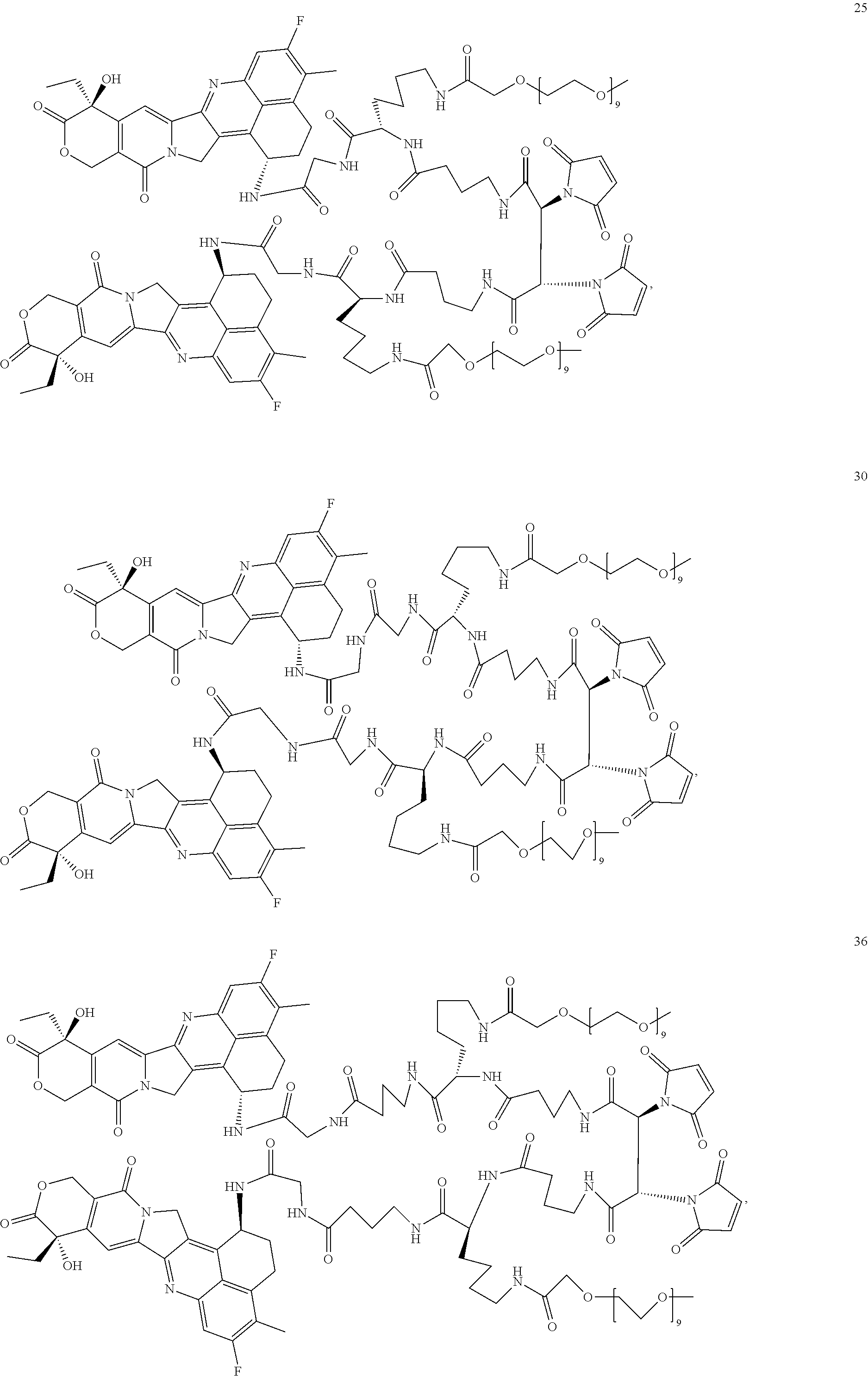

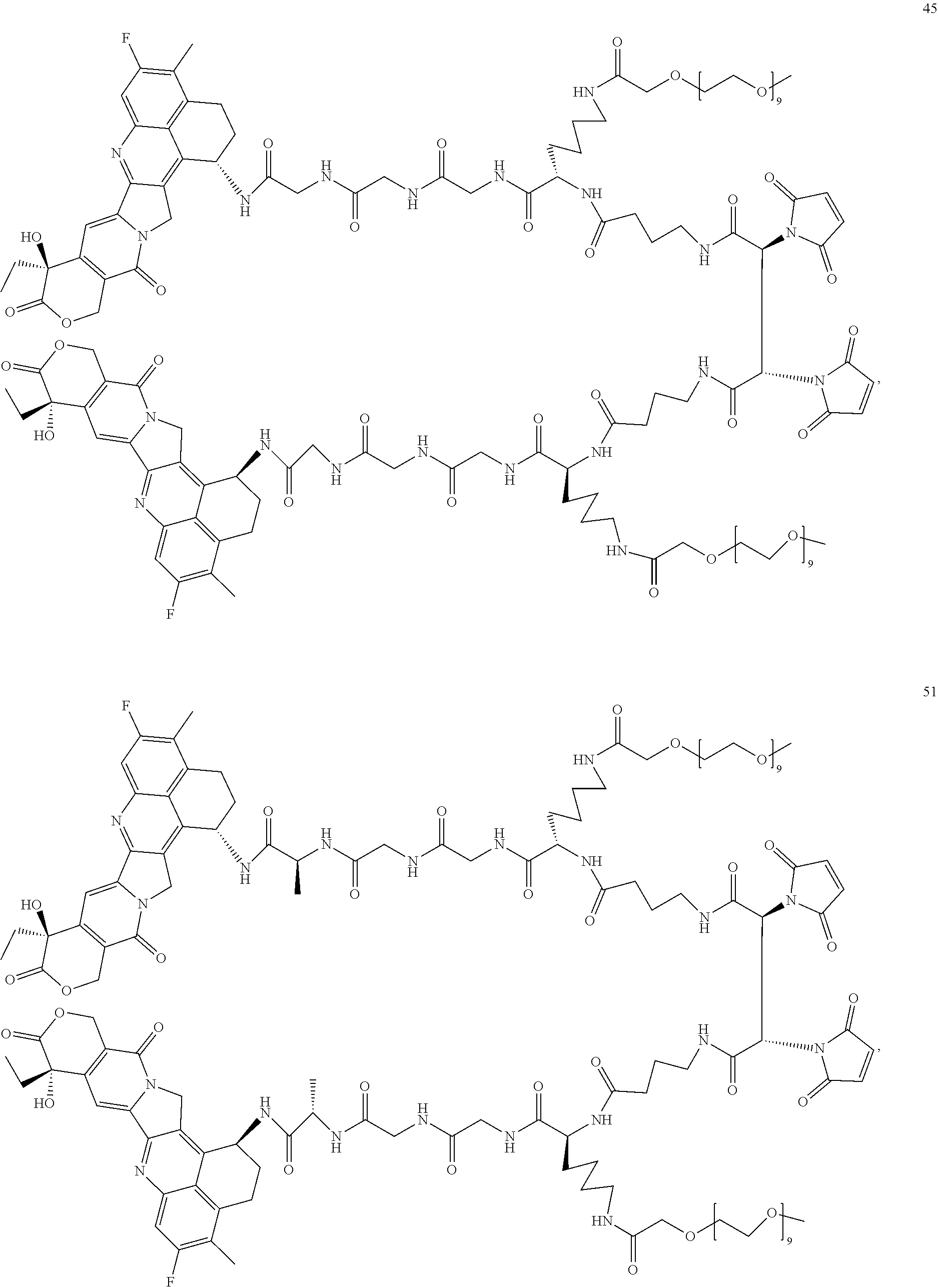

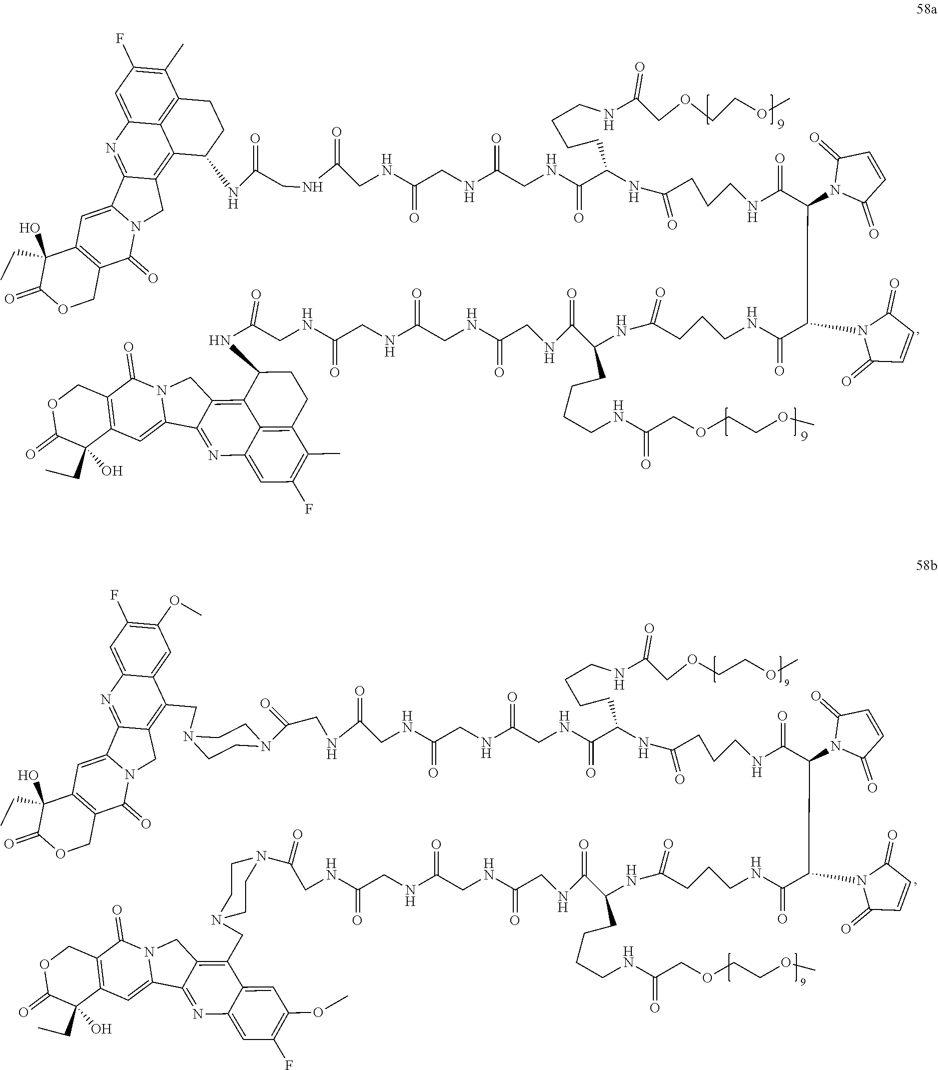

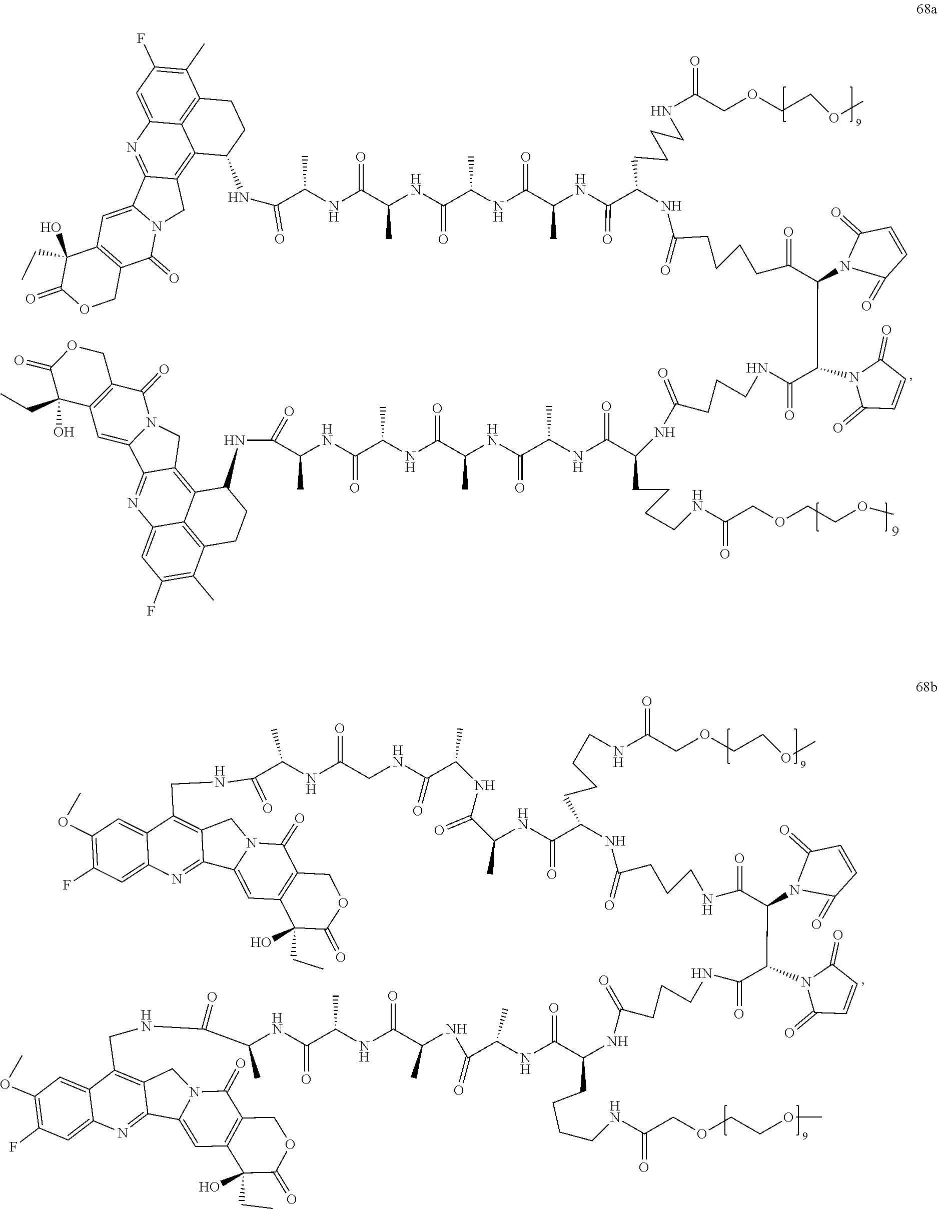

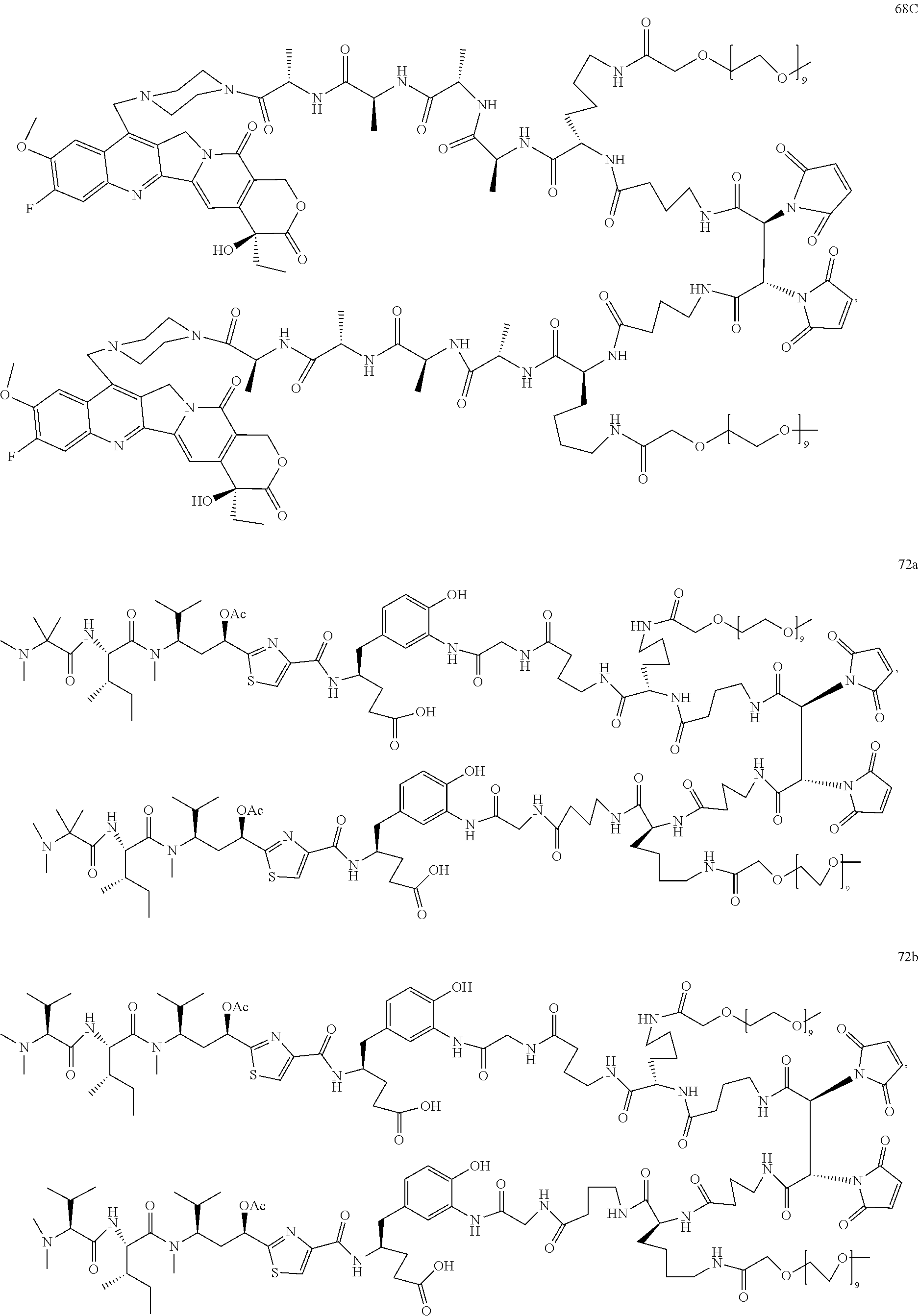

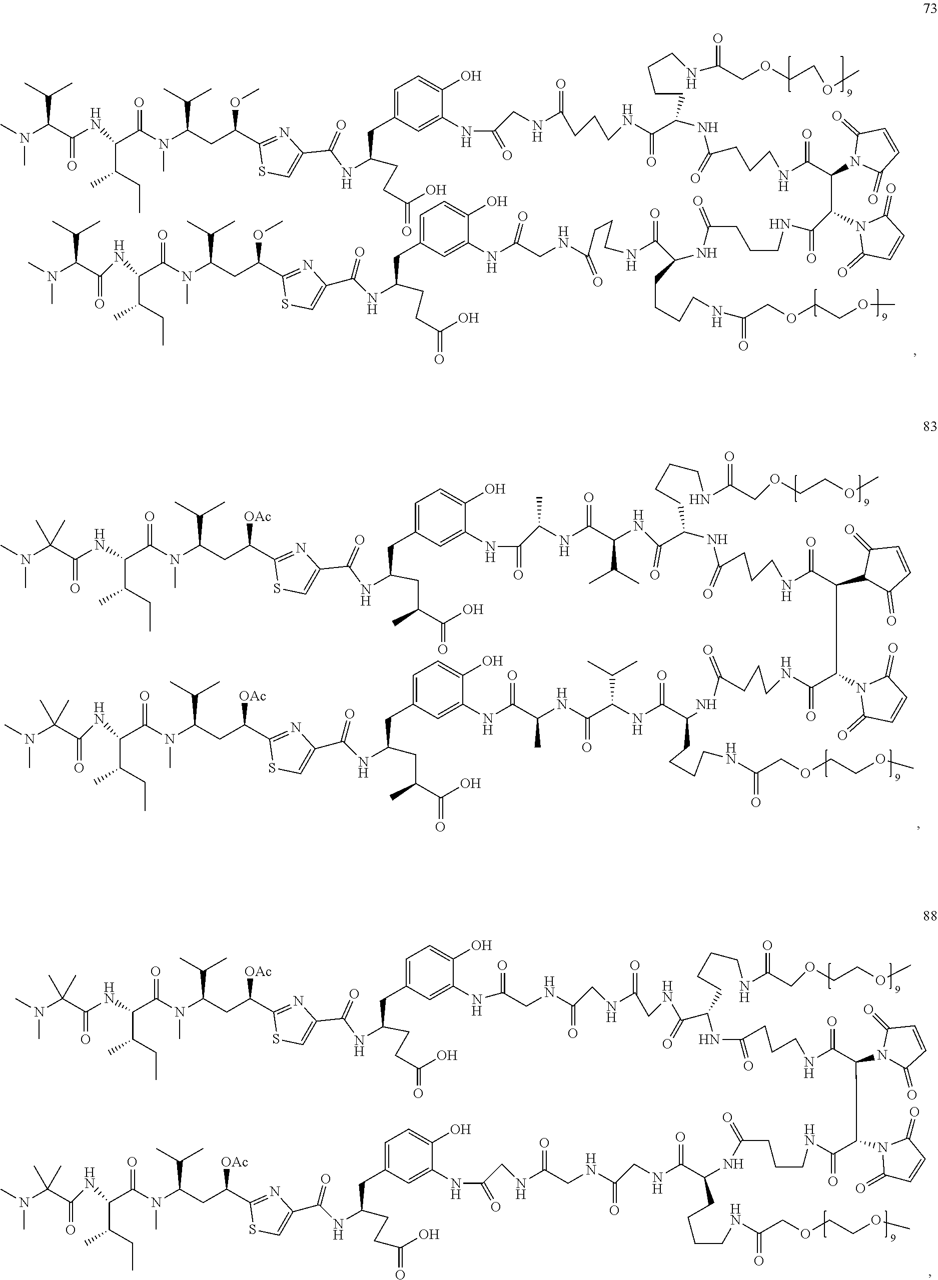

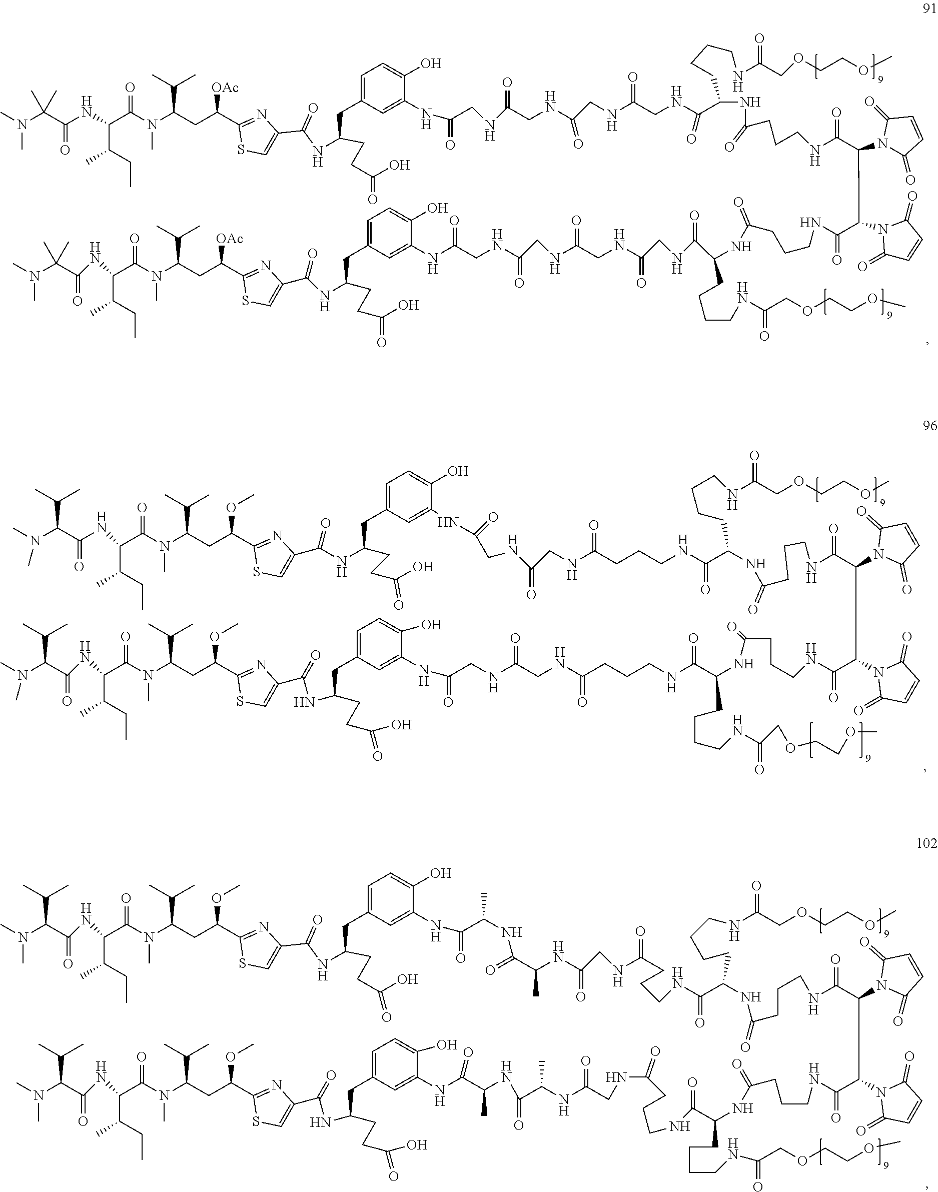

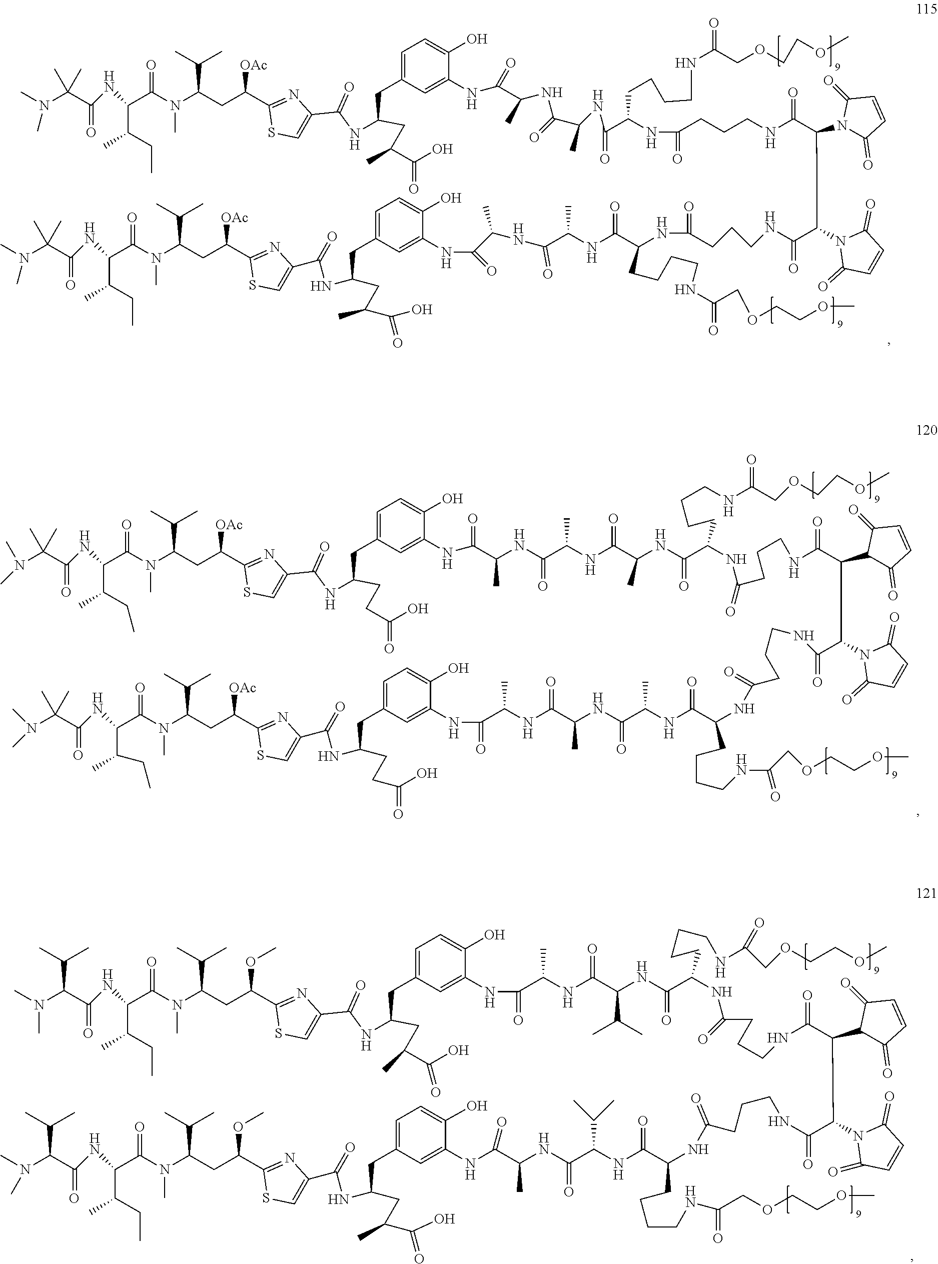

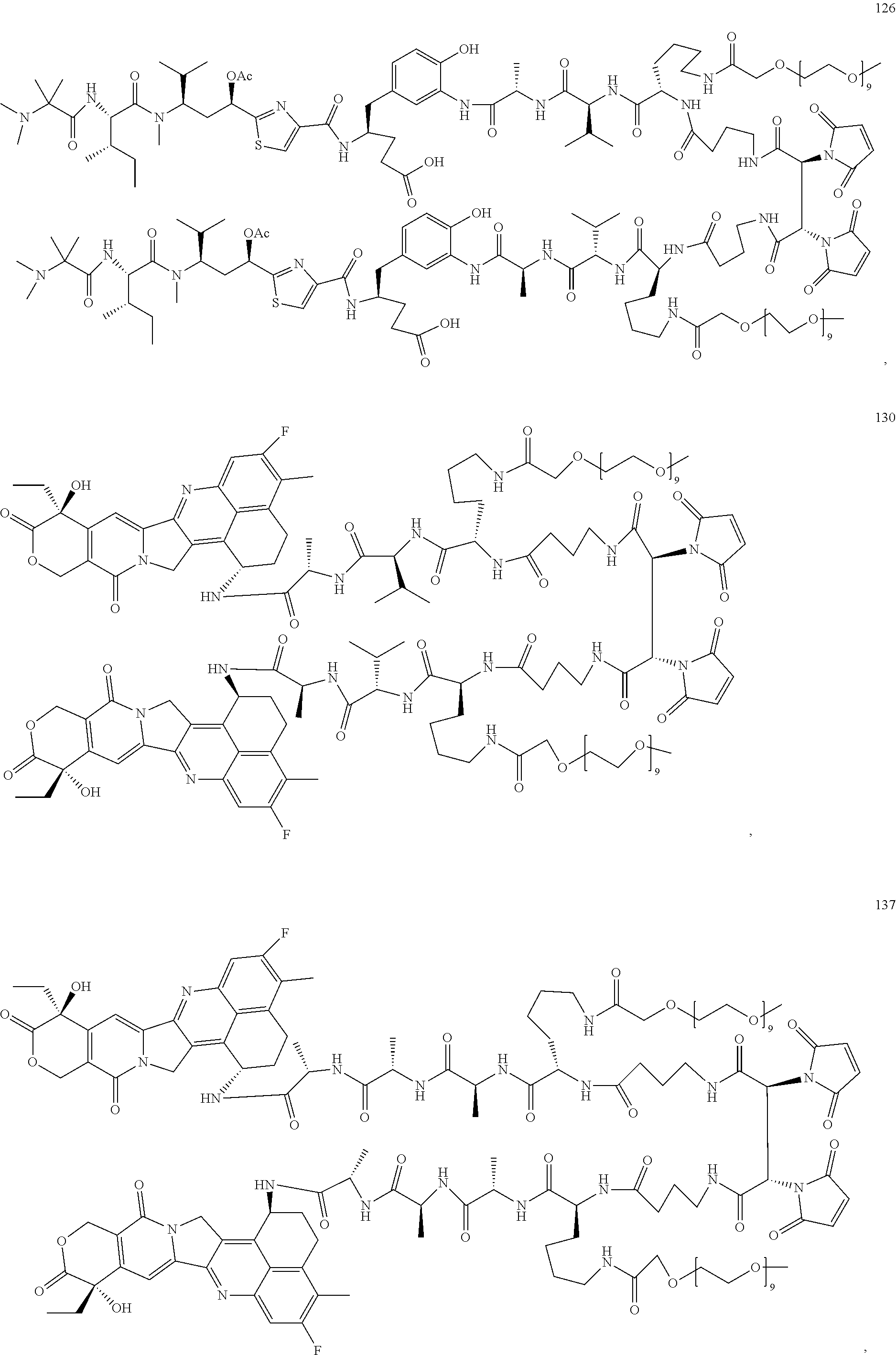

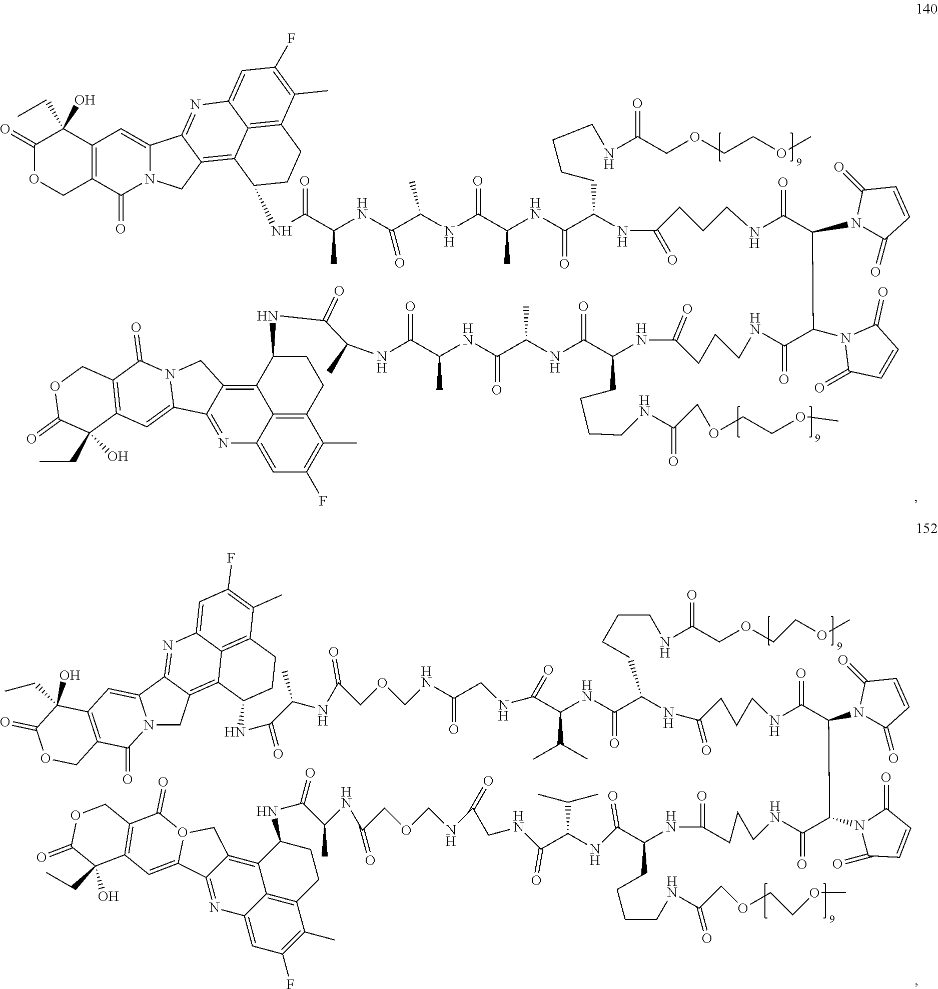

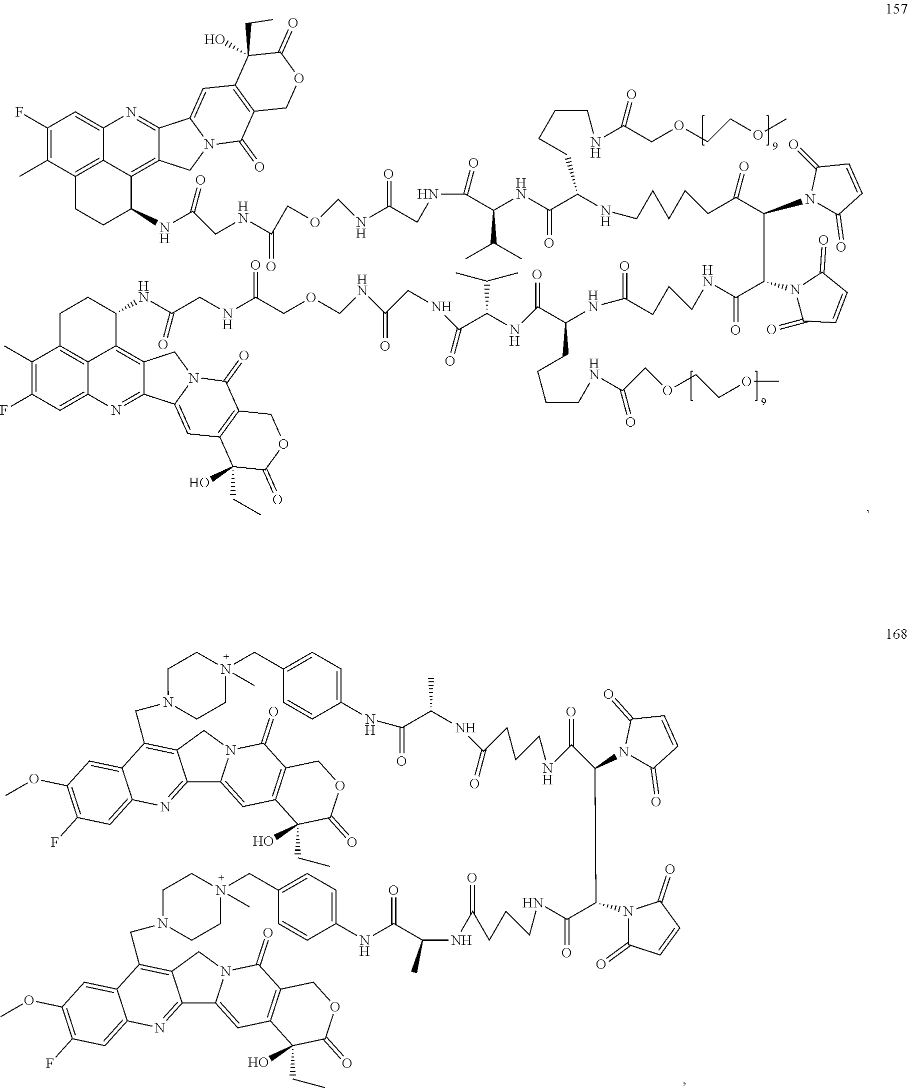

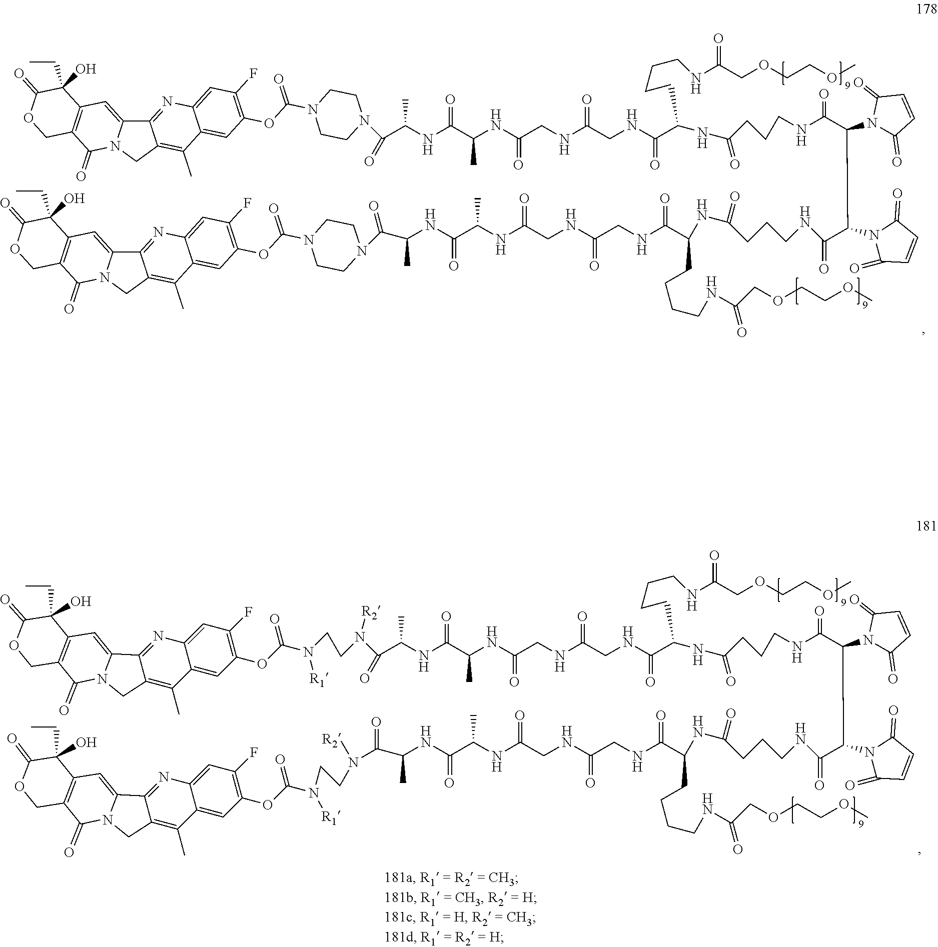

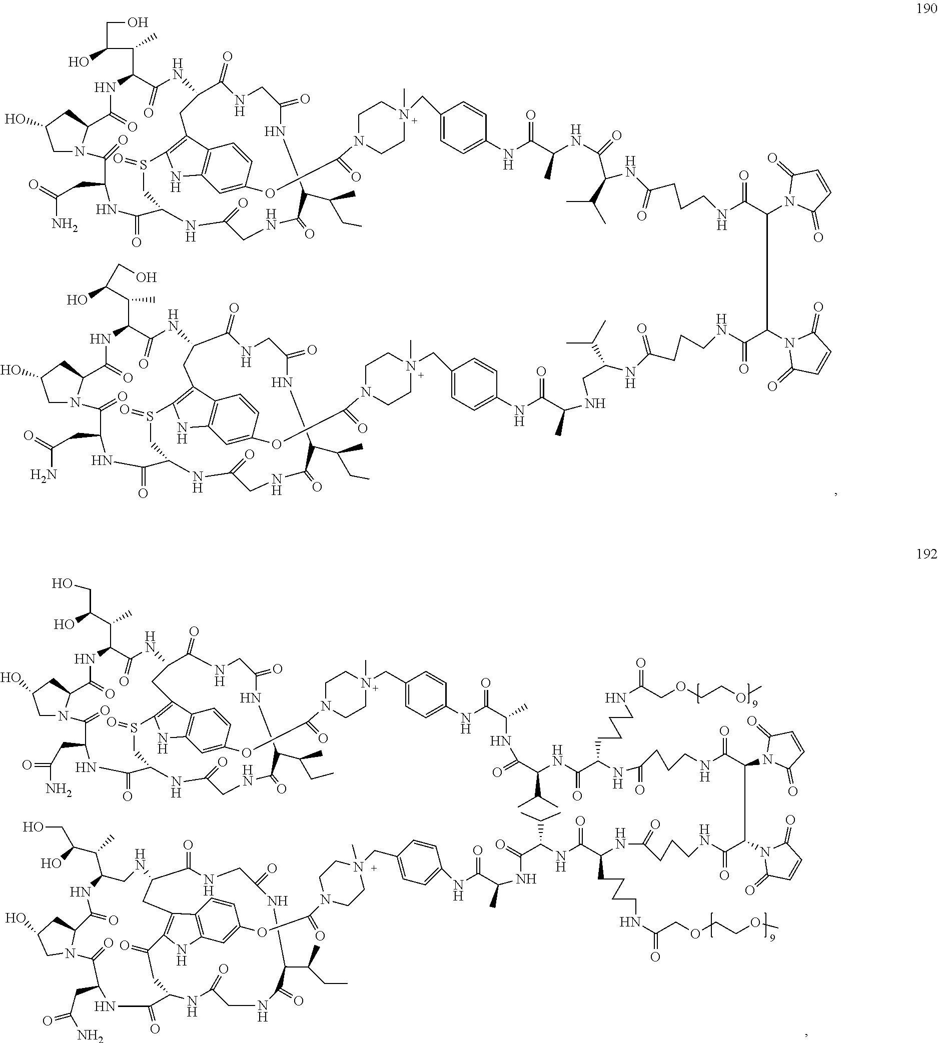

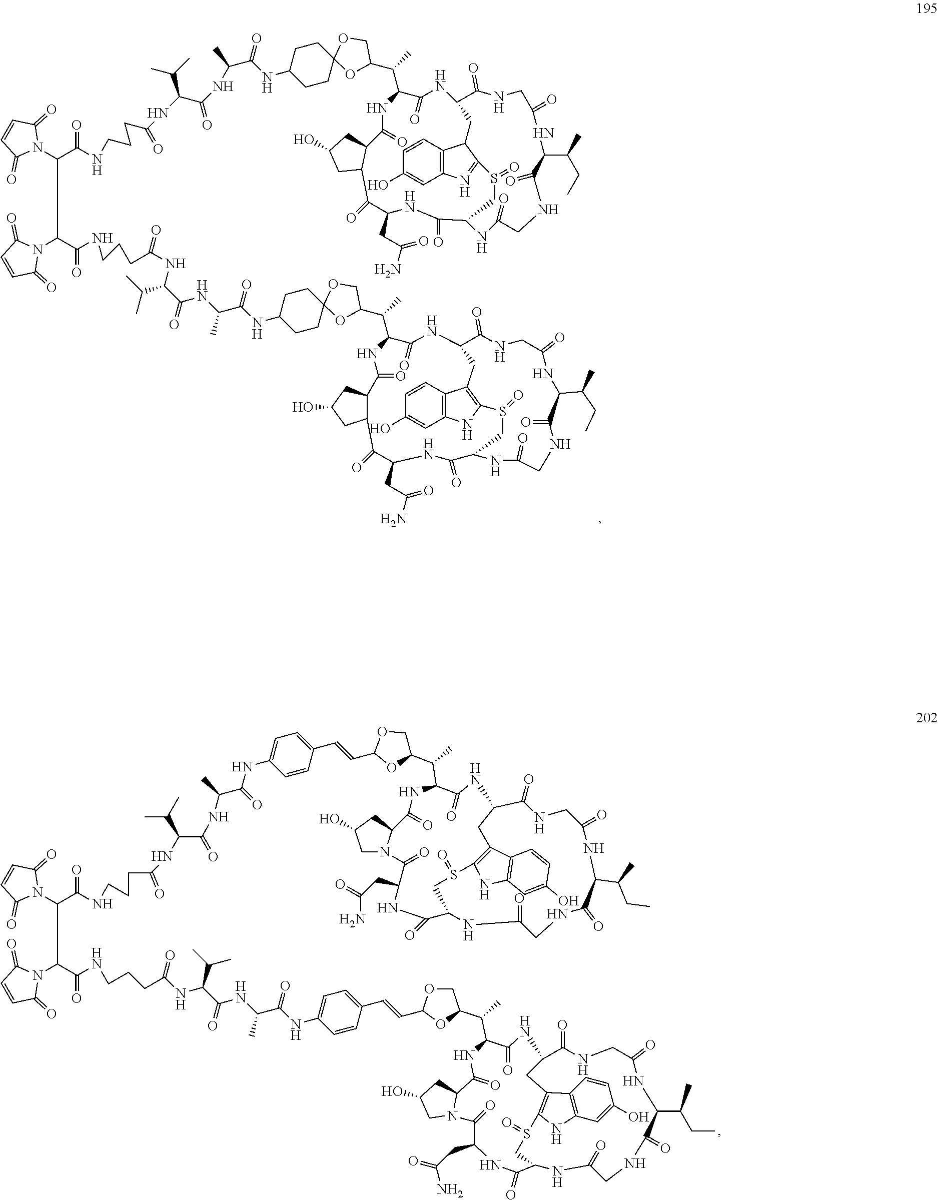

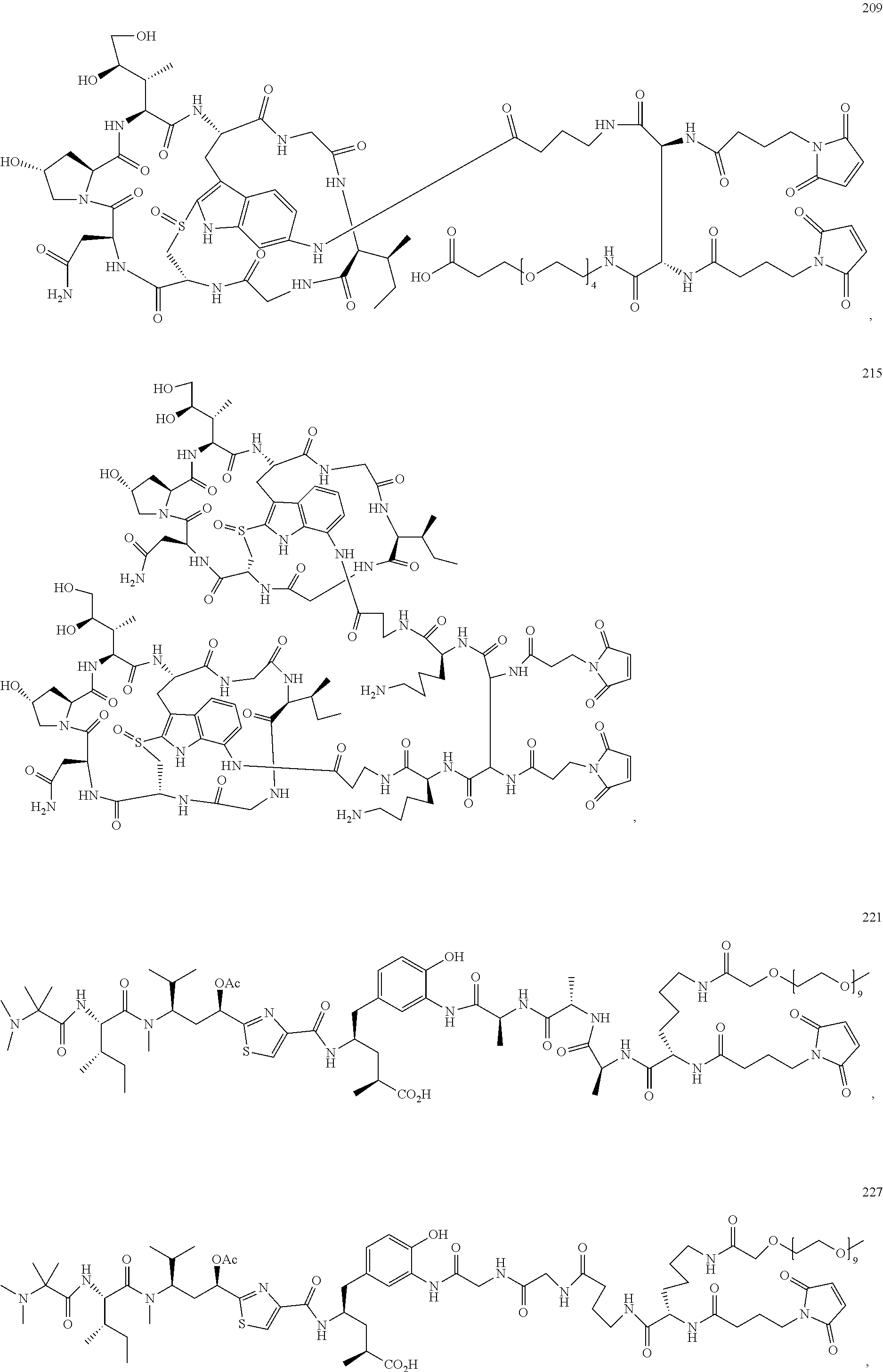

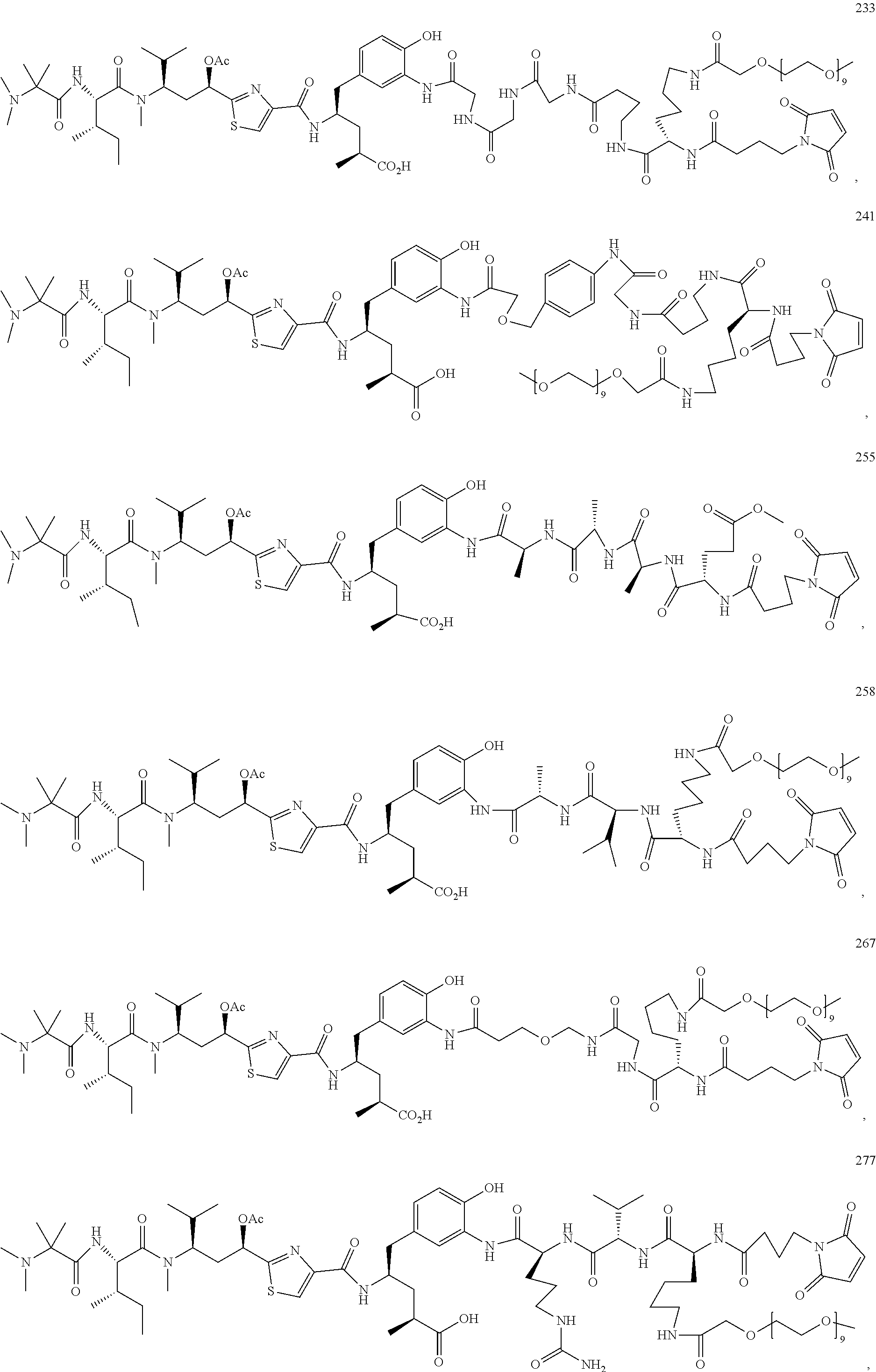

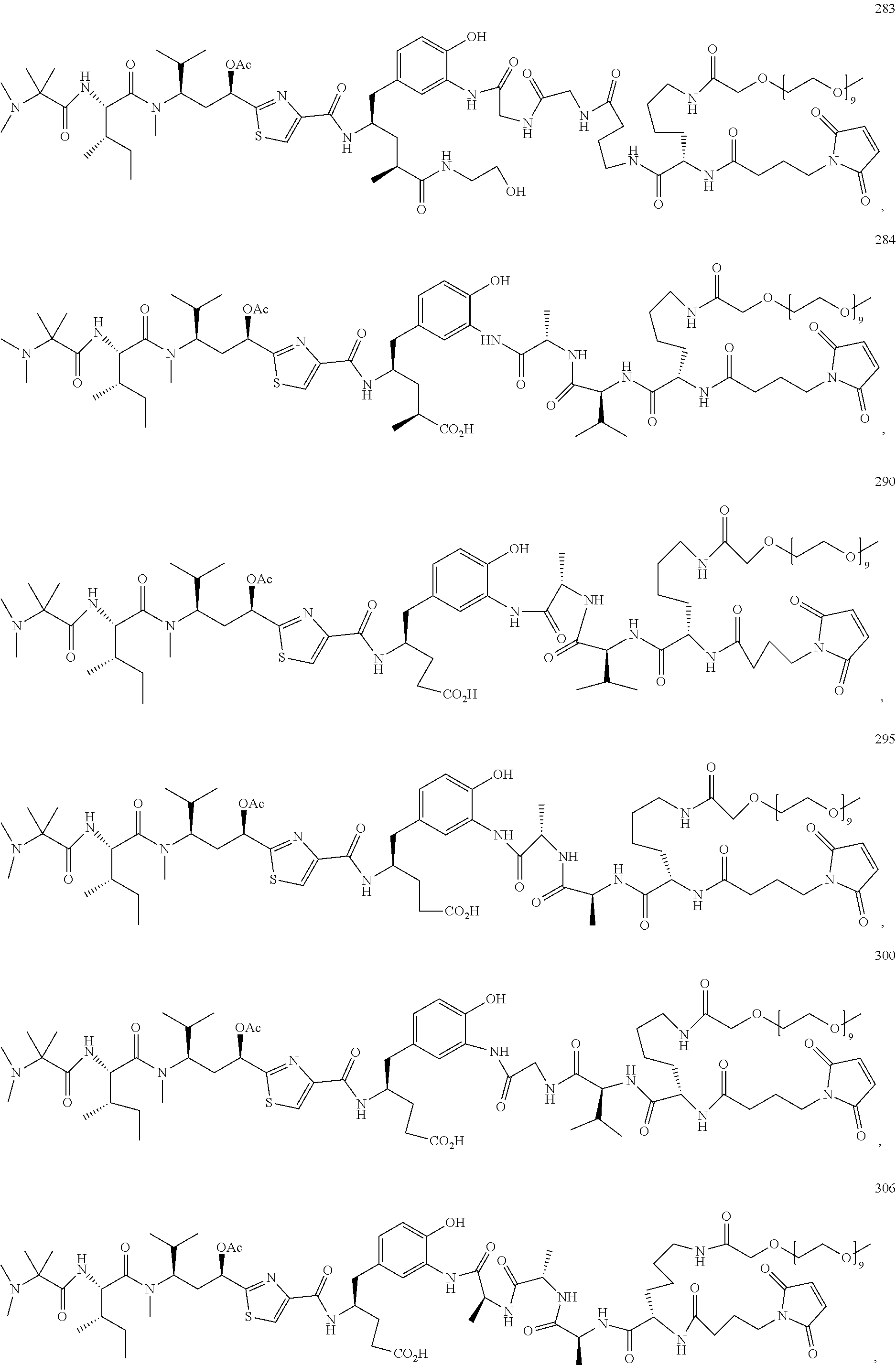

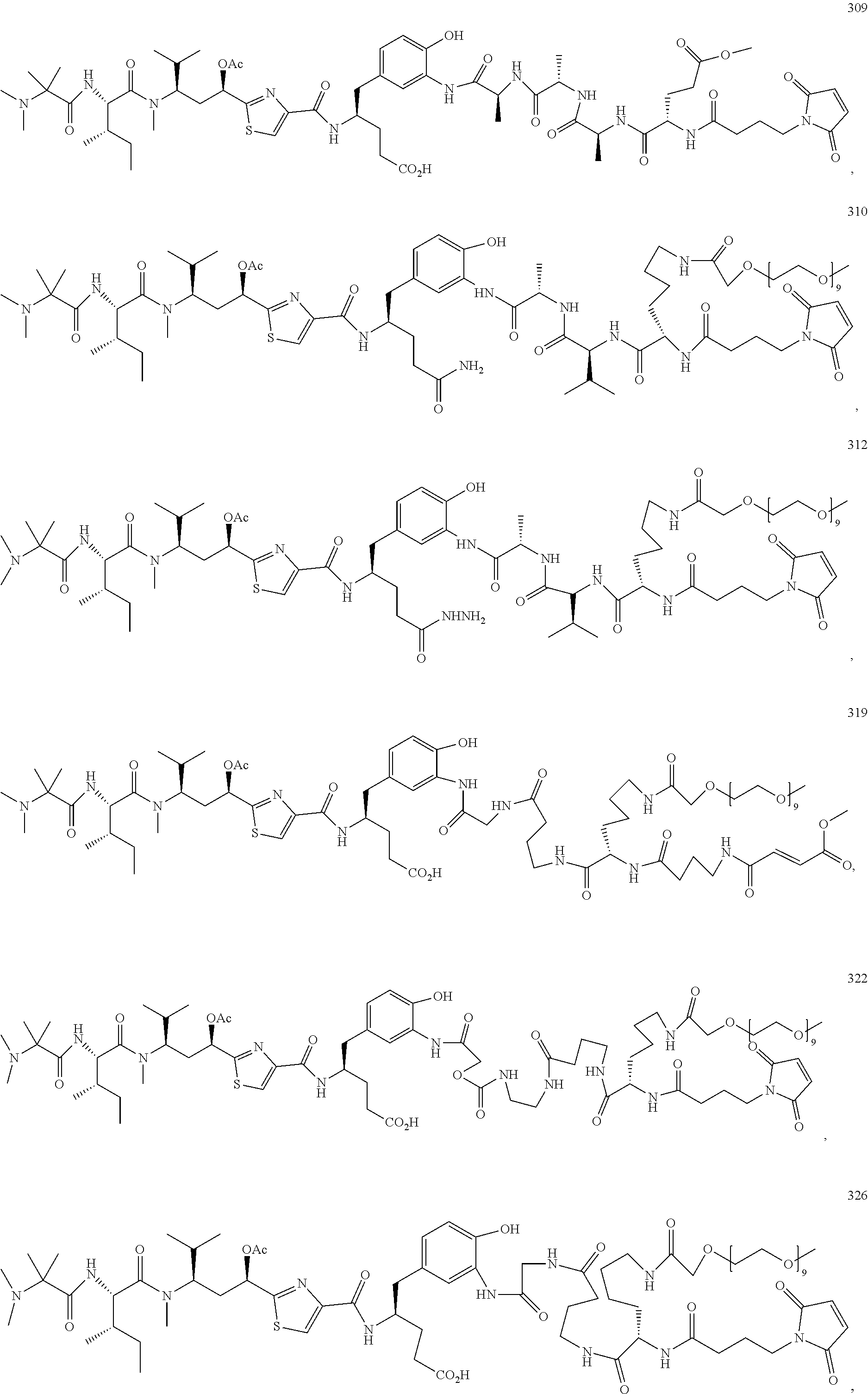

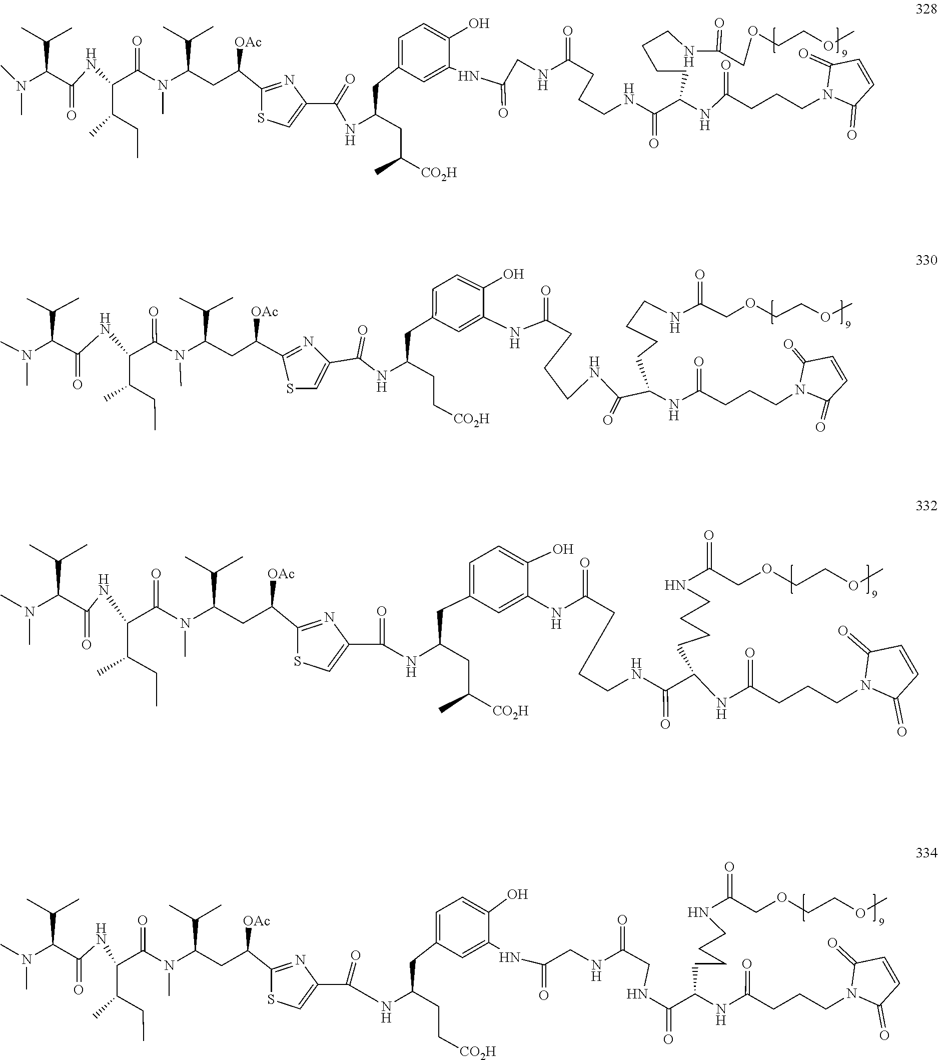

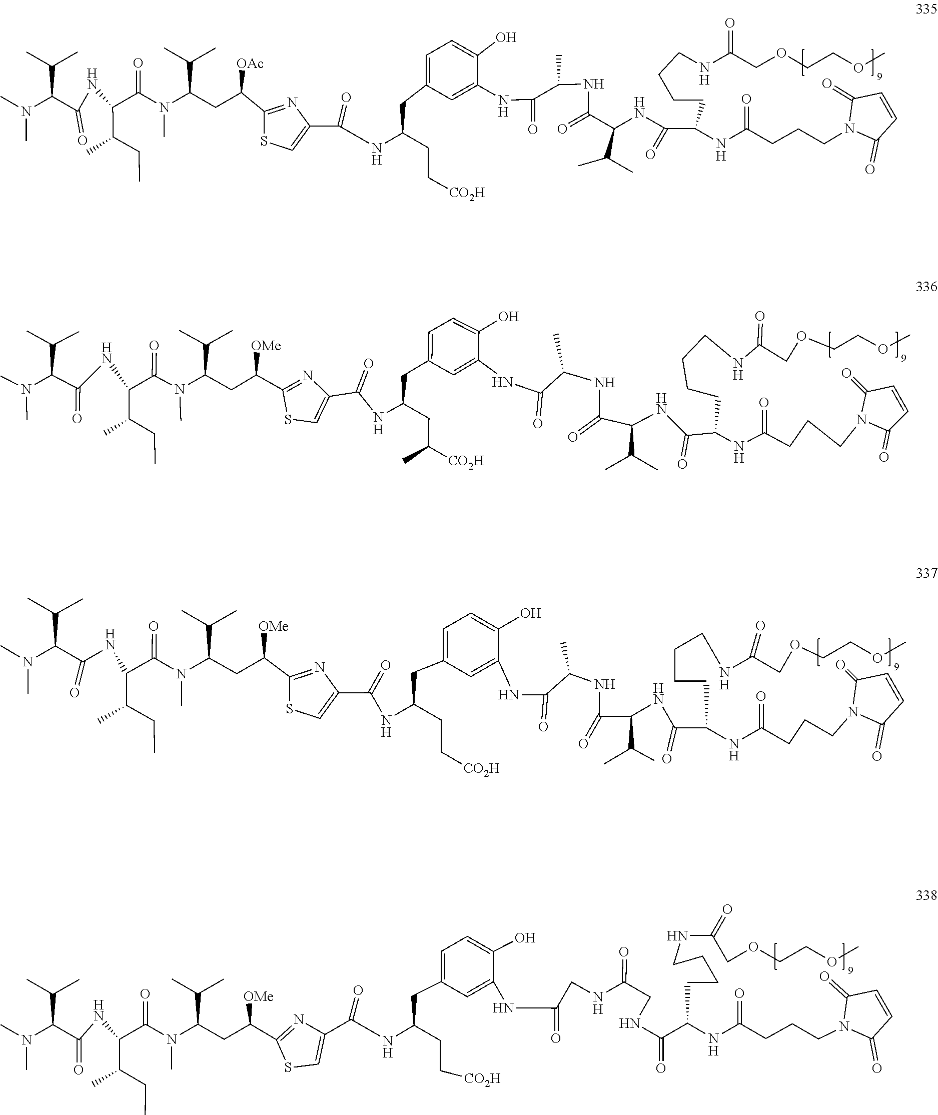

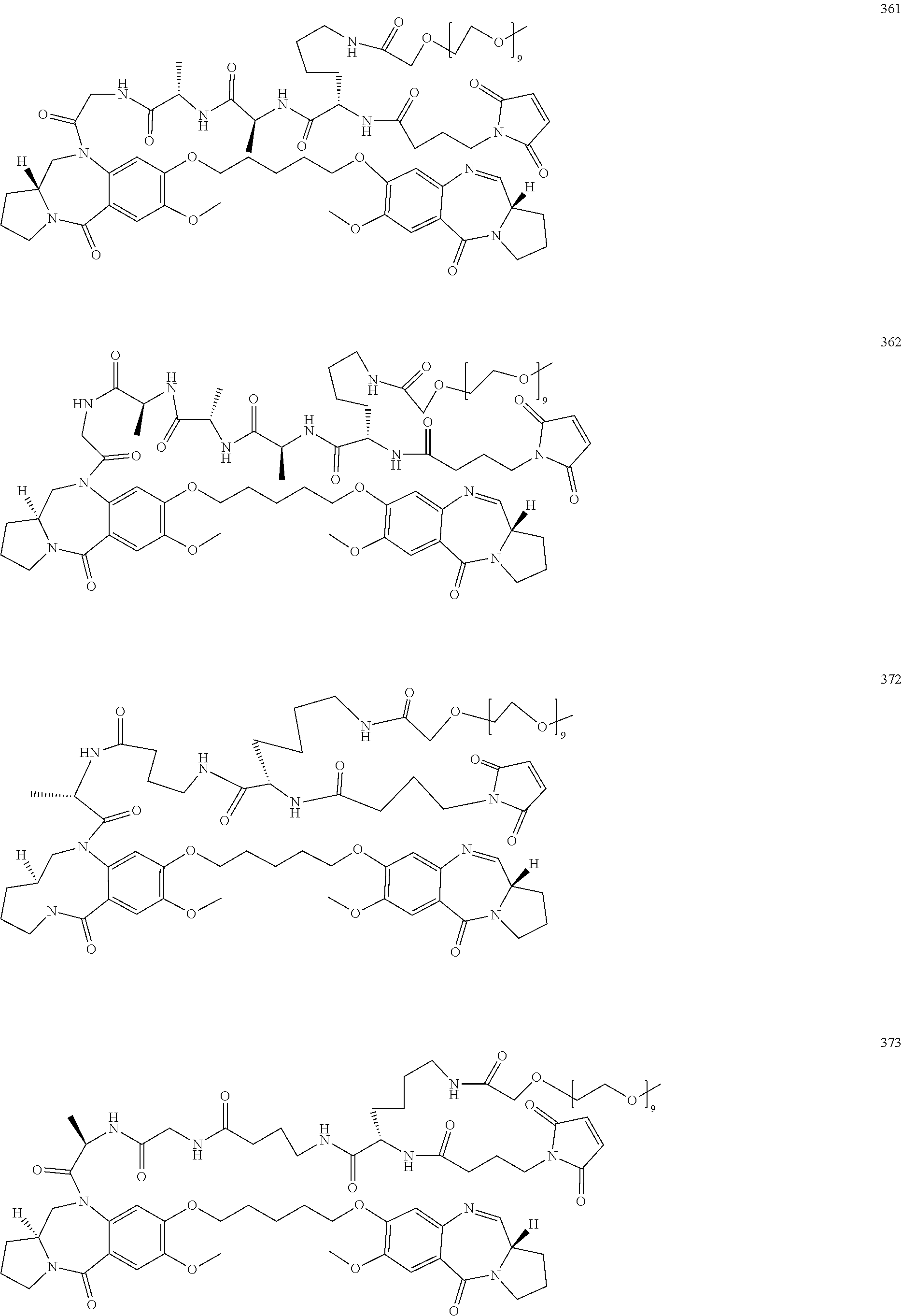

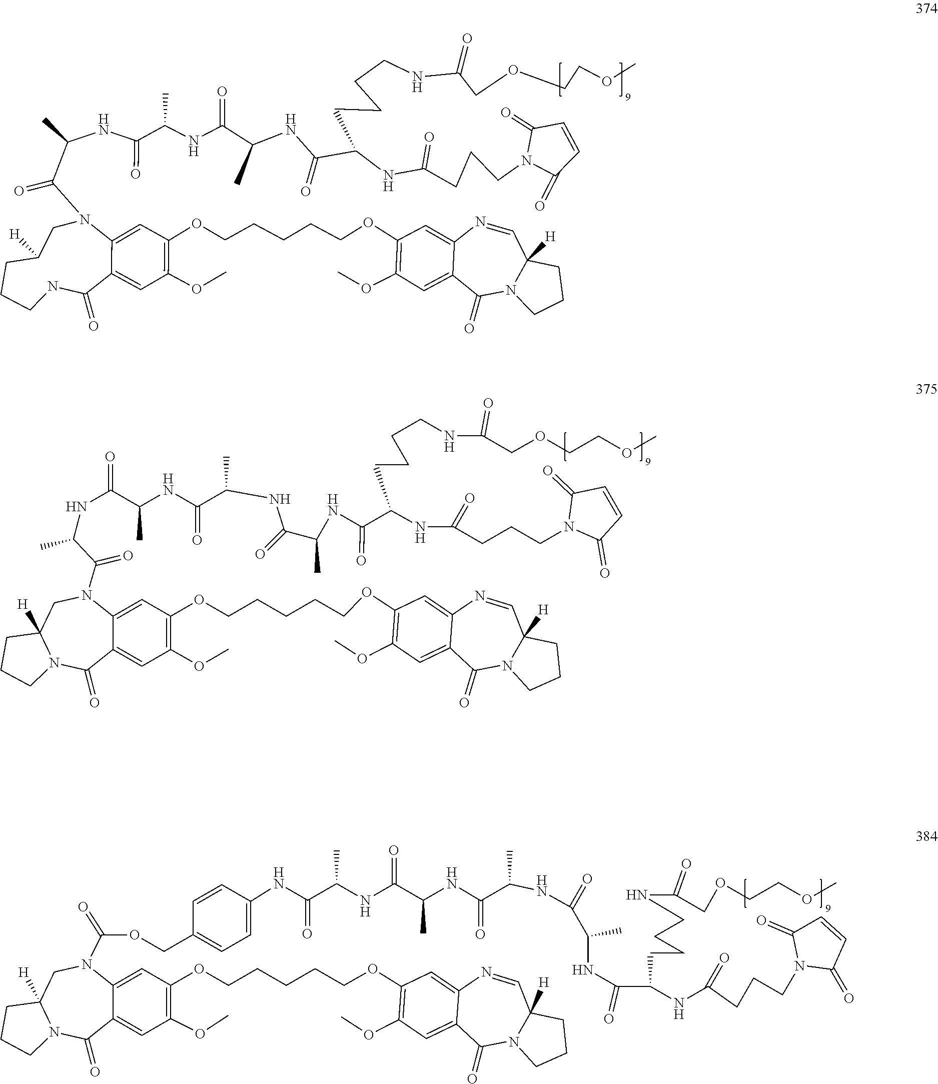

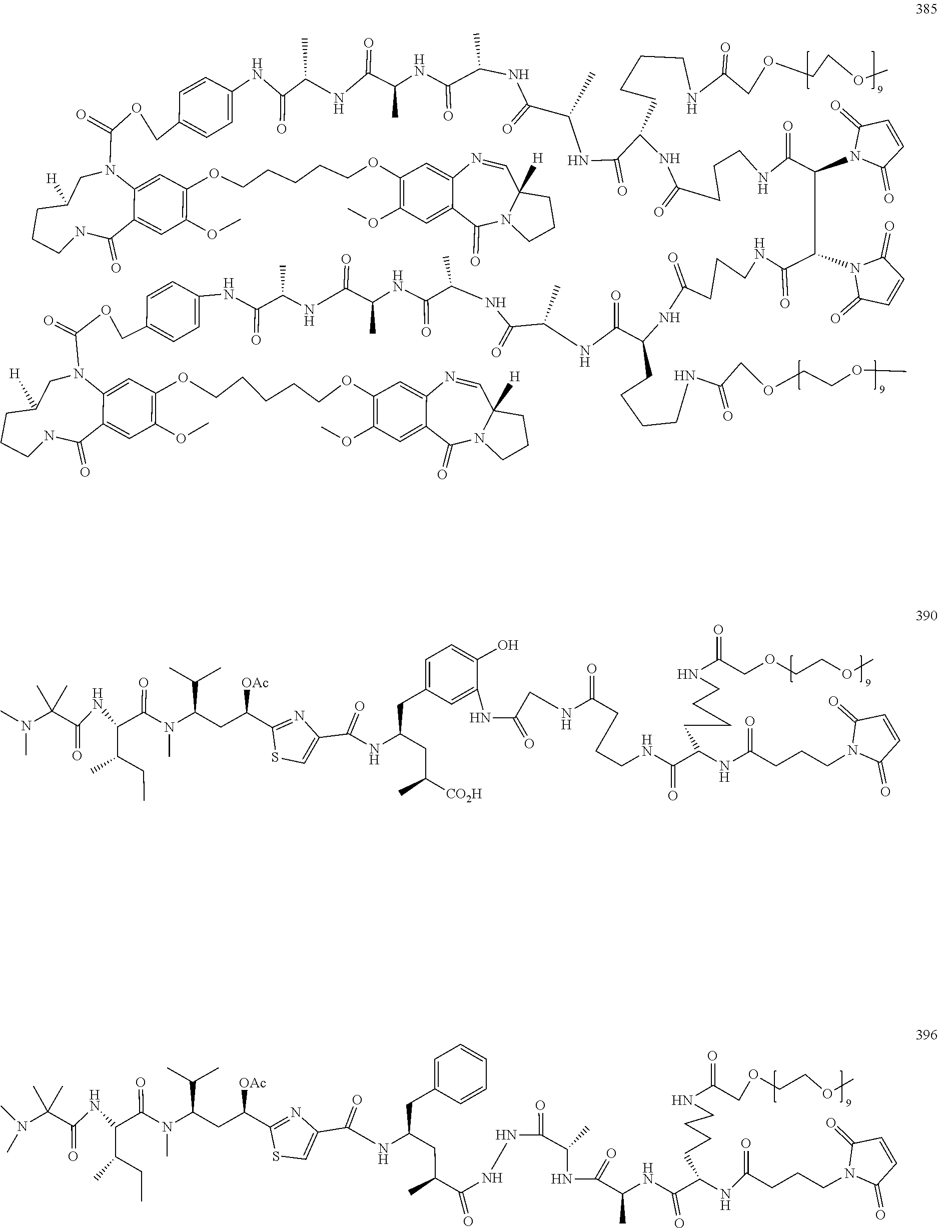

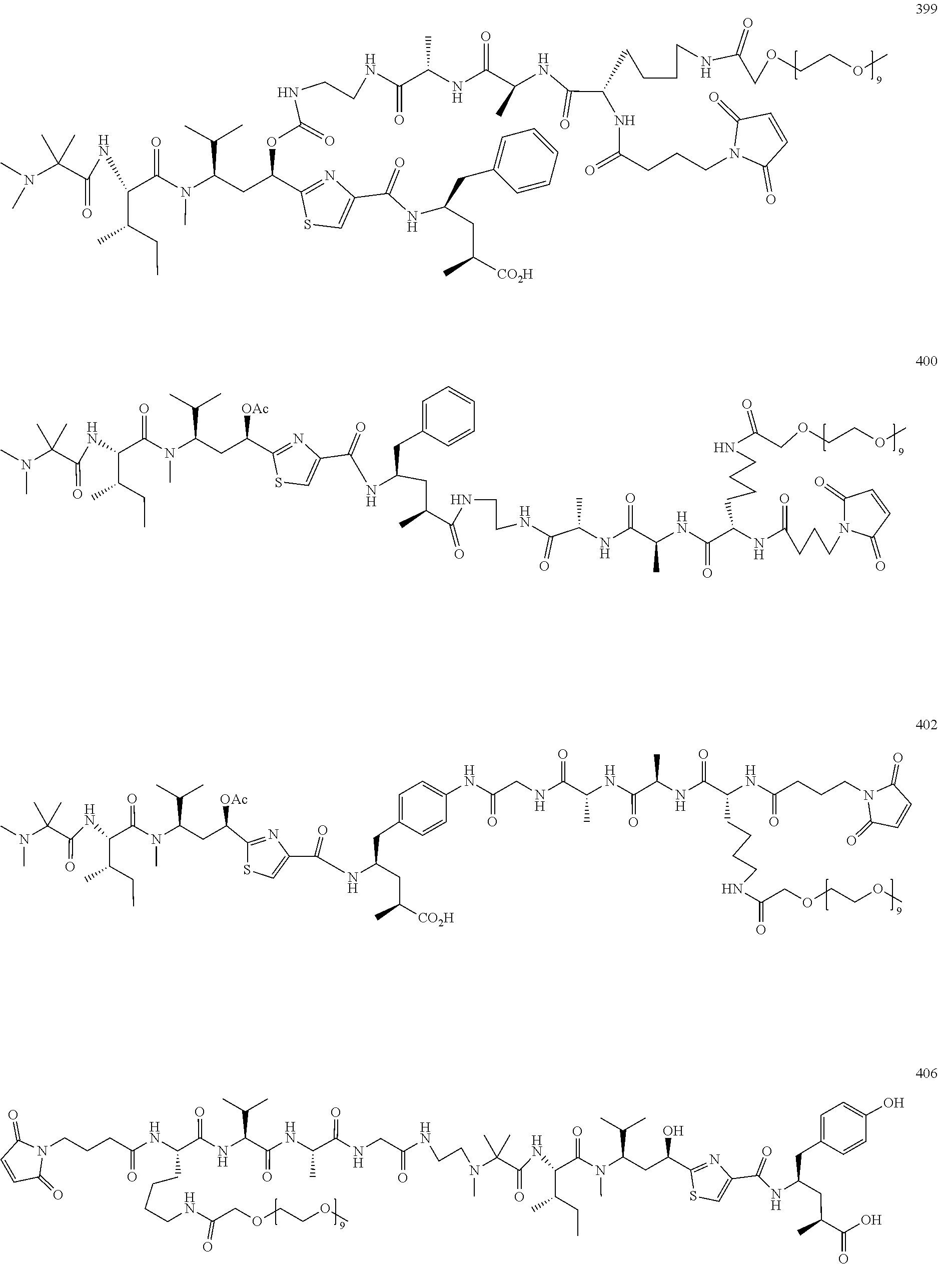

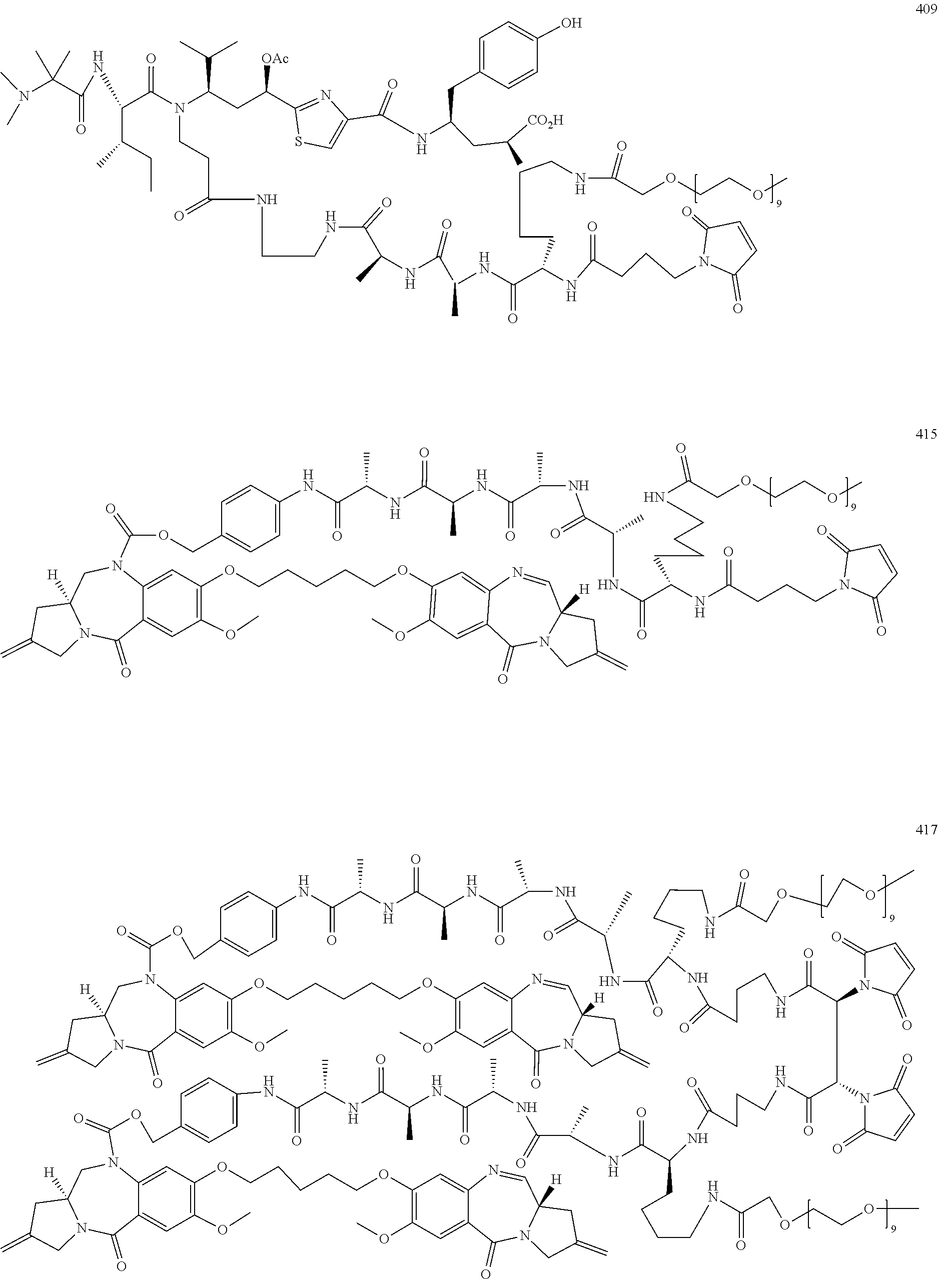

53. A pharmaceutical composition comprising a therapeutically effective amount of the BCMA antibody or antigen binding fragment thereof of claim 31, and a pharmaceutically acceptable salt, carrier, diluent, or excipient therefor, or a combination of the conjugates thereof.

54. The pharmaceutical composition according to claim 53, which is either in a liquid formula or in a formulated lyophilized solid/powder, comprising by weight of: 0.010%-99% of the BCMA antibody; 0.0%-0.0% of one or more polyols; 0.0%0% of one or more surfactants; 0.000-5.0% of one or more preservatives; 0.0%-30% of one or more amino acids; 0.0%-5.0% of one or more antioxidants; 0.00%-0.3% of one or more metal chelating agents; 0.00%-30.0% of one or more buffer salts for adjusting pH of the formulation to pH 4.5-7.5; and 0.00%-30.0% of one or more of isotonic agent for adjusting osmotic pressure between from about 250 to 350 mOsm when being reconstituted for administration to a patient;
wherein the polyol is selected from fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts);
wherein the surfactant is selected from polysorbate 20, polysorbate 40, polysorbate 65, polysorbate 80, polysorbate 81, or polysorbate 85, poloxamer, poly(ethylene oxide)-poly(propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; orisostearyl ethylimidonium ethosulfate; polyethyl glycol, polypropyl glycol, and copolymers of ethylene and propylene glycol;
wherein the preservative is selected from benzyl alcohol, octadecyldimethylbenzyl ammonium chloride, hexamethonium chloride, benzalkonium chloride, benzethonium chloride, phenol, butyl and benzyl alcohol, alkyl parabens such as methyl or propyl paraben, catechol, resorcinol, cyclohexanol, 3-pentanol, or m-cresol;
wherein the amino acid is selected from arginine, cystine, glycine, lysine, histidine, ornithine, isoleucine, leucine, alanine, glycine glutamic acid or aspartic acid;
wherein the antioxidant is selected from ascorbic acid, glutathione, cystine or and methionine;
wherein the chelating agent is selected from EDTA or EGTA;
wherein the buffer salt is selected from sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salts;
wherein the tonicity agent is selected from mannitol, sorbitol, sodium acetate, potassium chloride, sodium phosphate, potassium phosphate, trisodium citrate, or sodium chloride.

55. The pharmaceutical composition according to claim 53, which is packed in a vial, bottle, pre-filled syringe, or pre-filled auto-injector syringe, in a form of a liquid or lyophilized solid.

56. The BCMA antibody or antigen binding fragment thereof of claim 31, having in vitro, in vivo or ex vivo cell killing activity.

57. A method for synergistically treatment of multiple myeloma cells, B-cell mediated or plasma cell mediated disease, comprising administering the pharmaceutical composition of the BCMA antibody or antigen binding fragment thereof of claim 31, concurrently with a synergistic agent selected from a chemotherapeutic agent, a radiation therapy, an immunotherapy agent, an autoimmune disorder agent, an anti-infectious agents or another conjugate.

58. The method according to claim 57, wherein the synergistic agent is selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Azacitidine, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelinacetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.

59. A method for treatment of a medical disorder in a human subject, wherein the medical disorder is associated with presence of pathogenic B cells expressing B cell maturation antigen (BCMA), the method comprising administering to the human subject the BCMA antibody or antigen binding fragment thereof of claim 31,
wherein the medical disorder associated with presence of pathogenic B cells is a cancer of plasma cells or a cancer of B lymphocytes.
